(19) United States
(12) Patent Application Publication
(10) Pub. No.: US 2015/0023989 A1
(43) Pub. Date: Jan. 22, 2015

(54) NEW ANTIBODY DRUG CONJUGATES (ADCS) AND THE USE THEREOF

(71) Applicant: SEATTLE GENETICS, INC., Bothell, WA (US)

(72) Inventors: Hans-Georg Lerchen, Leverkusen (DE); Stefanie Hammer, Berlin (DE); Axel Harrenga, Wuppertal (DE); Charlotte Christine Kopitz, Berlin (DE); Carl Friedrich Nising, Leverkusen (DE); Anette Sommer, Berlin (DE); Beatrix Stelte-Luowig, Wulfrath (DE); Christoph Mahlert, Wuppertal (DE); Joachim Schuhmacher, Wuppertal (DE); Sven Golfier, Berlin (DE); Simone Greven, Dormagen (DE); Sandra Bruder, Wuppertel (DE)

(21) Appl. No.: 14/364,203

(22) PCT Filed: Dec. 12, 2012
(86) PCT No.: PCT/EP2012/075277

§ 371 Date: Jun. 10, 2014

(30) Foreign Application Priority Data
Dec. 14, 2011 (EP) 11193617.5
Oct. 22, 2012 (EP) 12189467.9

(51) Int. Cl. A61K 47/48; A61K 38/05

(52) U.S. Cl. CPC A61K 47/48561; A61K 38/05
USPC 424/179.1; 530/391.9; 548/467; 544/63

(57) ABSTRACT

The present application relates to new antibody drug conjugates (ADCs) of N,N dialkylauristatins directed against the target FGFR2, drug metabolites of said ADCs, a method for producing said ADCs, the use of said ADCs for the treatment and/or prevention of illnesses as well as the use of said ADCs for producing pharmaceuticals for the treatment and/or prevention of illnesses, particularly of hyperproliferative and/or angiogenic diseases such as carcinosis. Such treatments can be carried out as monotherapy or in combination with other pharmaceuticals or additional therapeutic measures.

The present application relates to new binder-drug conjugates (ADCs) of N,N-dialkylauristatins that are directed against the target fibroblast growth factor receptor 2 (FGFR2), to active metabolites of these ADCs, to processes for preparing these ADCs, to the use of these ADCs for treating and/or preventing illnesses, and also to the use of these ADCs for producing medicaments for treating and/or preventing illnesses, more particularly hyperproliferative and/or angiogenic diseases such as, for example, cancer diseases. Such treatments may be practised as a monotherapy or else in combination with other medicaments or further therapeutic measures.

Cancer diseases are the consequence of uncontrolled cell growth in a wide variety of tissues. In many cases the new cells penetrate existing tissue (invasive growth), or they metastase into remote organs. Cancer diseases occur in a wide variety of organs, and the illnesses often progress in a tissue-specific manner. The designation “cancer disease” as a generic term therefore describes a large group of defined diseases of different organs, tissues and cell types.

Early-stage tumours may be able to be removed by surgical and radiotherapeutic measures. Metastasized tumours can generally only be given palliative therapy by means of chemotherapeutic agents. The objective in that case is to achieve the optimum combination of improving quality of life and prolonging remaining lifetime.

The majority of the chemotherapeutic agents which are presently administered parenterally are often not target-directed at the tumour tissue or the tumour cells, but instead, as a result of their systemic administration, are distributed non-specifically within the body, hence including at locations at which exposure to the drug is undesirable, such as in healthy cells, tissues and organs, for example. This may lead to unwanted side-effects and even to serious effects of general toxicity, which then often greatly limit the therapeutically useful dose range of the drug, or necessitate complete cessation of medication.

The improved and selective availability of these chemotherapeutic agents in the tumour cell or the immediately surrounding tissue, and the associated boost in effect, on the one hand, and minimization of toxic side-effects, on the other hand, have therefore been a focal point for a number of years in the development of new chemotherapeutic agents. Many attempts have been made to date to develop efficient methods of introducing the drug into the target cell. Optimizing the association between drug and intracellular target and minimizing the intercellular distribution of drug, to adjacent cells, for example, nevertheless continue to constitute a difficult problem.

Monoclonal antibodies, for example, are suitable for the target-directed addressing of tumour tissue and tumour cells. The significance of such antibodies for the clinical treatment of cancer diseases has seen a considerable general increase in recent years, based on the activity of such agents as trastuzumab)(Herceptin®, rituximab (Rituxan®), cetuximab (Erbitux®) and bevacizumab (Avastin®), which have since been approved for the therapy of individual, specific tumour diseases [see e.g. G. P. Adams and L. M. Weiner, Nat. Biotechnol. 23, 1147-1157 (2005)]. Consequently there has also been a marked increase in interest in so-called immunoconjugates such as, for example, the aforementioned ADCs, in which an internalizing antibody directed against a tumour-associated antigen is joined covalently via a linking unit (“linker”) to a cytotoxic agent. Following introduction of the ADC into the tumour cell and subsequent cleavage of the conjugate, either the cytotoxic agent itself or another metabolite with cytotoxic activity, formed from the cytotoxic agent, is released within the tumour cell, where it is able to develop its effect directly and selectively. In this way it would be possible to keep the damage to normal tissue within significantly closer limits in comparison to a conventional chemotherapy of the cancer disease [see e.g. J. M. Lambert, Curr. Opin. Pharmacol. 5, 543-549 (2005); A. M. Wu and P. D. Senter, Nat. Biotechnol. 23, 1137-1146 (2005); P. D. Senter, Curr. Opin. Chem. Biol. 13, 235-244 (2009); L. Ducry and B. Stump, Bioconjugate Chem. 21, 5-13 (2010)].

Instead of antibodies, it is also possible for binders from the small-molecule drug sphere to be used as binders which bind selectively to a specific target location (“target”), such as to a receptor, for example [see e.g. E. Ruoslahti et al., Science 279, 377-380 (1998); D. Karkan et al., PLoS ONE 3 (6), e2469 (Jun. 25, 2008)]. Also known are conjugates of cytotoxic drug and addressing ligand that exhibit a defined cleavage point between ligand and drug for the release of the drug. A “predetermined break point” of this kind may exist, for example, within a peptide chain which can be cleaved selectively at a particular site by a specific enzyme at the location of action [see e.g. R. A. Firestone and L. A. Telan, US Patent Application US 2002/0147138].

The activity of monoclonal antibodies is demonstrated in various types of cancer. Thus HERCEPTIN® and Erbitux® are used successfully in the treatment of HER2-positive breast cancer and EGFR-positive colorectal cancer, respectively.

The coupling of cytotoxic compounds to antibodies forms an extended possibility for additionally improving cancer therapy, since these conjugates allow tumour-specific toxophore accumulation and at the same time reduce the systemic toxicity. With respect to activity and tolerance, clinical studies with brentuximab vedotin in Hodgkin's lymphoma and with trastuzumab-DM1 in breast cancer have yielded highly promising results, which support the development of new antibodies and new ADCs against other tumour antigens.

Antibody-based therapy proves to be very powerful in the treatment of various carcinomas, including solid tumours. Thus, for example Herceptin® has been deployed successfully in the treatment of breast cancer, and Rituxan® is powerful in forms of carcinoma associated with the B cells. At the focal point of the development of a successful antibody-based therapy is the isolation of antibodies against cell surface proteins which are expressed preferably on tumour cells.

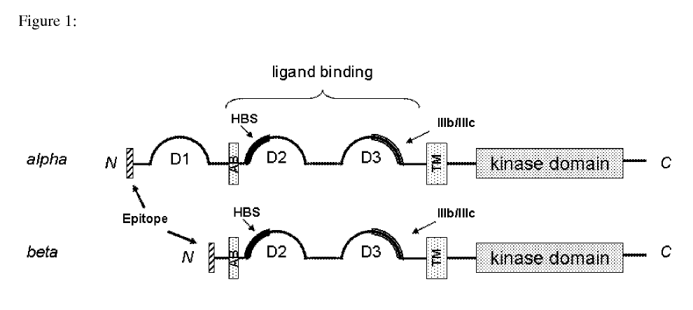

The fibroblast growth factor receptors are tyrosine receptor kinases (RTK), of which four are known in mammals (FGFR1, FGFR2, FGFR3, FGFR4). As ligands, 22 human fibroblast growth factors (FGF) have been identified (Eswarakumar and Schlessinger, Cytokine & Growth Factor Reviews 2005, 16:139-149; Shimada et al., Proc. Natl. Acad. Sci. USA 2001, 98:6500-6505). The FGFRs consist of three extracellular immunoglobulin (Ig)-like domains, namely D1-D3, with domains 2 and 3 being necessary for ligand binding, and also of an individual transmembrane domain and a cytoplasmic domain, which contains the catalytic centre of the protein tyrosine kinase (a schematic illustration is given in FIG. 1).

Schematic illustration of the structure of FGFR2. Alpha (SEQ ID NO: 1) and beta (SEQ ID NO: 2) splice variants are compared with one another. The illustration shows the three Ig-like domains (D1, D2 and D3), the transmembrane domain (TM) and the intracellular kinase domain. The heparin binding site (HBS), the acidic box (AB), and the alternative IIIb/IIIc domains are likewise labelled. The amino terminus is marked by N, the carboxy terminus by C.

The extracellular component additionally harbours the acidic box (AB) and the heparin binding site (HBS) (see FIG. 1). An important feature of the FGFR family of RTKs is that there are different, alternatively spliced variants in existence. The full-length FGFR is identified as FGFR alpha (SEQ ID NO: 1), while the isoform, which is missing D1, is identified as FGFR beta (SEQ ID NO: 2) (FIG. 1). An alternative splicing in domain 3 leads to two different variants, namely FGFR2 IIIb, which is encoded by the exons 7 and 8, and FGFR2 IIIc, which is encoded by the exons 7 and 9 (FIG. 1). The latter splicing influences the ligand binding, leading to the specificity pattern. FGFR2 IIIc is expressed primarily by mesenchymal cells, and FGFR2 IIIb essentially by epithelial cells. FGF7 is also known as keratinocyte growth factor (KGF), and binds only to FGFR2 IIIb, which is therefore also called KGFR. When the FGFs bind to their receptors, there is subsequent dimerization and phosphorylation of the FGFRs, and a downstream signalling via the FRS-GRB2 docking protein complex to the RAS-MAPK signal transduction cascade and the PI3K-AKT signal transduction cascade. The former signal transduction cascade is involved in cell growth and in cell differentiation, while the latter is involved in the survival of the cells and in the determination of cell fate (Katoh and Katoh, Int. J. Oncol. 2006, 29:163-168).

For correct organogenesis during embryogenesis, an orchestrated signal transduction of all four receptors (FGFR1 to FGFR4) and of their splicing variants via the different FGFs is necessary (Ornitz et al., Genome Biol 2001, 2:3005). In the case of FGFR2, the absence of all FGFR2 variants leads to defects in placental and limb bud formation, and therefore leads to fatality at the E10.5 stage. Specific knock-out of FGFR2 IIIb in the mouse leads likewise to fatality (at P0) in association with agenesis of the lungs, of the anterior lobe, of the thyroid gland, of the teeth and of the limbs, whereas disruption of the FGFR2 IIIc variant is survivable, with incidence of retarded ossification, proportional dwarfism and synostosis of the base of the skull (Eswarakumar and Schlessinger, 2005). Activating mutations of FGFR2 in the germ track lead to severe malformations during embryogenesis, such as coronal synostosis and cranial synostosis in the case of Apert's syndrome or in the case of Pfeiffer's syndrome in humans (Robin et al., in Gene Reviews, NCBI Bookshelf Wash., edited by Pagon et al., 1993). In adults, FGFR2 signal transduction is involved in wound healing, in epithelial repair and in the protection of cells of the skin and mucous membrane (Braun et al., Phil. Trans. R. Soc. Lond. B 2004, 359:753-757) and in regeneration in the event of liver damage (Steiling et al., Oncogene 2003, 22:4380-4388; Böhm, dissertation, Eidgenössische Technische Hochschule Zürich, 2009). A role of FGFR2 signal transduction in the migration of EPDC (epicardial derived cells) into the heart following infarction is therefore under discussion because during embryogenesis, FGF10-FGFR2 signal transduction is necessary for the migration of EPDC into the compact myocardium, a process which is necessary for the development of the heart (Vega-Hernández et al., Development 2011:3331-3340; Winter and De Groot, Cell Mol. Life Sci. 2007, 64:692-703). All of these roles played by FGFR2 are regenerative in nature, and apparently of essential importance only under non-physiological conditions, as a result of a disruption in tissue homeostasis. Increased somatic signal transduction via FGFR2 is involved in various pathological conditions such as acne (Katoh, J. of Invest. Dermatol. 2009, 129:1861-1867), psoriasis (Finch et al., Am. J. Pathol. 1997, 151:1619-1628; Xu et al., J. Invest. Dermatol. 2011:131:1521-1529) and cancer (see below).

A number of studies have been published which show a strong association of FGFR2 expression with an adverse outcome in cancer patients:

The overexpression of FGFR2 and/or KGF is accompanied by expansive growth of stomach carcinoma and shorter survival of the patients (Matsunobu et al., Int. J. Cancer 2006, 28:307-314; Toyokawa et al., Oncol. Reports 2009, 21:875-880). The overexpression of FGFR2 was detected in 31-36.5% of all stomach carcinoma samples examined (Matsunobu et al., Int. J. Cancer 2006, 28:307-314; Toyokawa et al., Oncol. Reports 2009, 21:875-880). Adenocarcinoma (70% of all stomach carcinomas) is subdivided, additionally, into two different pathological types, namely stomach cancer of the intestinal type of and of the diffuse type. Interestingly, the first, less aggressive type is associated with an activated ErbB2 signal pathway, whereas, in the case of the latter, more aggressive type, aberrations occur in the FGFR2/PI3K signal pathway (Yamashita et al., Surg. Today 2011, 41:24-38). FGFR2 overexpression occurred in 53% of all samples of stomach cancer of the diffuse kind (Yamashita et al., Surg. Today 2011, 41:24-38). Drawing together all of the data, HER2 expression and FGFR2 expression appear to occur in two different patient populations. To some extent possibly, the expression of FGFR2 is the result of a gene amplification, since amplifications of FGFR2 are found in approximately 7-10% of all primary stomach carcinomas (Kunii et al., Cancer Res. 2008, 68:23-40-2348). Moreover, FGFR2 expression has not only been found in metastases, but was in fact even greater in metastases than in primary tumours (Yamashita et al., Surg. Today 2011, 41:24-38).

In the case of breast cancer, FGFR2 IIIb expression was found in 57% of tumour samples, but hardly at all in healthy tissue (Tamaru et al. 2004, 84:1460-1471). KGF (FGF7) occurred in 45% of random samples, generally together with FGFR2 IIIb. The co-expression of FGF7 and its single receptor FGFR2 IIIb was associated with a significantly reduced number of apoptotic cells within the primary tumour by comparison with primary breast carcinomas, where neither FGF7 nor FGFR2 IIIb was expressed (Tamaru et al. 2004, 84:1460-1471). As in the case of stomach cancer, a gene amplification was found for breast cancer as well, and also in 4% of triply negative breast carcinomas (TNBC) (Turner et al., Oncogene 2010, 29:2013-2023). In breast cancer, a number of changes in individual nucleotides (Single Nucleotide Polymorphism, SNP) have been identified that are associated with an increased risk of breast cancer (Hunter et al., Nature Genetics 2007, 6:870-874). If the SNPs are localized within Intron 2, this leads to a transcriptional upregulation of FGFR2 (Katoh, Expert Reviews 2010, 10:1375-1379). Interestingly, FGFR1 is upregulated preferentially in the case of oestrogen receptor (ER) positive breast carcinomas, while FGFR2 is upregulated in the case of ER-negative breast carcinomas (Katoh, Expert Reviews 2010, 10:1375-1379).

In the case of pancreatic carcinoma, the overexpression of FGFR2 IIIb and/or FGF7 is heavily correlated with venal invasion (Cho et al., Am. J. Pathol. 170:1964-1974), with co-expression of FGFR2 and FGF7 having been found in tumour cells, but occurring even more frequently in the stroma cells which are adjacent to the tumour cells (Ishiwata et al., Am. J. Pathol. 1998, 153:213-222).

In the case of epithelial ovary cancer, 80% of cases showed upregulation of FGRF2 as compared with normal tissue, and in 70% there was FGF7 in the ascitic fluid (Steele et al., Oncogene 20:5878-5887).

The FGFR2 protein was found in all invasive cervical carcinomas tested, with strong expression at the invasive front of the tumours (Kawase et al., Int. J. Oncol. 2010, 36:331-340).

In the case of pulmonary adenocarcinoma, co-expression of FGF7 and FGFR2 occurred in 51.6% of cases, and is correlated with lower degrees of differentiation, higher rate of proliferation, lymph node metastasis, and shorter 5-year survival (Yamayoshi et al., J. Pathol. 2004, 204:110-118).

In the case of endometrial carcinoma, activating FGFR2 mutations occur in approximately 16% of cases (Pollock et al., Oncogene 2007, 26:7158-7162).

In the case of oesophageal cancer (OC), co-expression of FGF7 and FGFR2 in cancer cells was found in 26% of patients, and was associated with a trend towards a shorter survival time (Yoshino et al., Int. J. Oncol. 2007, 31:721-728).

In the case of liver cell carcinoma, the expression of FGFR2 was upregulated 4.7 times more strongly in poorly differentiated tumours. This expression is associated with the incidence of portal vein invasion and lower disease-free survival times (Harimoto et al., Oncology 2010, 78:361-368).

A number of publications, with in vitro and in vivo experimental data, show a causal connection between altered FGFR2 signal transduction and tumour growth.

Knock-down or inhibition of FGFR2 in cells of stomach cancer (Takeda et al., Clin. Cancer Res. 2007; 13:3051-3057; Kunii et al., Cancer Res. 2008; 68:2340-2348), breast cancer (Turner et al., Oncogene 2010, 29:2013-2023), ovarian cancer (Cole et al., Cancer Biol. Ther. 2010, 10:495-504) and squamous carcinoma of the head and neck (Marshall et al., Clin. Cancer Res. 2011, 17:5016-5025) led to reduced proliferation or increased apoptosis of the tumour cells. In tumour xenotransplants as well, knock-down of FGFR2 and inhibition of FGFR2 in tumour cell lines which overexpress FGFR2 was observed to cause growth inhibition in stomach cancer cell lines (Takeda et al., Clin. Cancer Res. 2007; 13:3051-3057) and ovarian cancer cell lines (Cole et al., Cancer Biol. Ther. 2010, 10:495-504). Moreover, FGF7, which exclusively activates FGFR2, increases the proliferation of stomach cancer cell lines (Shin et al., J. Cancer Res. Clin. Oncol. 2002, 128:596-602), breast cancer cell lines (Zhang et al., Anticancer Res. 1998, 18:2541-2546) and ovarian cancer cell lines (Cole et al., Cancer Biol. Ther. 2010, 10:495-504) in vitro and in vivo. Furthermore, knock-down of FGFR2 in endometrial cancer cell lines which contain FGFR2 with activating mutations likewise led to the standstill of the cell cycle and to the induction of cell death (Byron et al., Cancer Res. 2008, 68:6902-6907).

FGFR2 signal transduction promotes the migration and invasion of stomach cancer cell lines (Shin et al., J. Cancer Res. Clin. Oncol. 2002, 128:596-602), breast cancer cell lines (Zhang et al., Anticancer Res. 1998, 18:2541-2546) and pancreatic cancer cell lines in vitro (Nomura et al., Br. J. Cancer 2008, 99:305-313; Niu et al., J. Biol. Chem. 2007, 282:6601-6011).

In the case of oesophageal cancer, FGFR2 is the most highly upregulated gene in tumour-associated fibroblasts. Isolated tumour-associated fibroblasts released a soluble factor which promotes the proliferation of oesophageal cancer cells (Zhang et al., hum. Cancer Biol. 2009, 15:4017-4022), thereby demonstrating that FGFR2 expressed by stroma cells is also able to promote tumour progression.

There are only a few reports on anti-FGFR2 antibodies. Fortin et al. (J. Neurosci. 2005, 25: 7470-7479) describe a blocking anti-FGFR2 antibody. Wei et al. (Hybridoma 2006, 25: 115-124) have shown antibodies with exclusive specificity for FGFR2 IIIb, which inhibit the KGF-induced cell proliferation. WO2007144893 describes inhibiting antibodies which bind FGFR2 and FGFR3. In WO2010054265 and in Zhao et al. (Clin. Cancer Res. 2010, 16:5750-5758), antibodies which inhibit FGF binding are described, including for example GAL-FR21 and GAL-FR22. Bai et al. (Cancer Res. 2010, 70:7630-7639) describe antibodies having specificity for FGFR2 IIIb. R&D Systems market anti-FGFR2 antibodies which have an activity-neutralizing effect in the manufacturer's assays. WO2005066211 describes antibodies which are directed against various cell-surface FGFRs, including FGFR2. WO2009100105 describes isoform-specific anti-FGFR2 antibodies which can be linked covalently to effector molecules. WO2007134210 describes methods for treating colorectal cancer using anti-FGFR2 antibodies or immunoconjugates. WO2007144893 describes FGFR2 antibodies with binding affinity for further FGFRs, which block the ligand-dependent and the constitutive ligand-independent FGFR2 receptor activation.

Auristatin E (AE) and monomethylauristatin E (MMAE) are synthetic analogues of the dolastatins, a specific group of linear pseudopeptides which were originally isolated from marine sources and which have in some cases very potent cytotoxic activity with respect to tumour cells [for a review see e.g. G. R. Pettit, Prog. Chem. Org. Nat. Prod. 70, 1-79 (1997); G. R. Pettit et al., Anti-Cancer Drug Design 10, 529-544 (1995); G. R. Pettit et al., Anti-Cancer Drug Design 13, 243-277 (1998)].

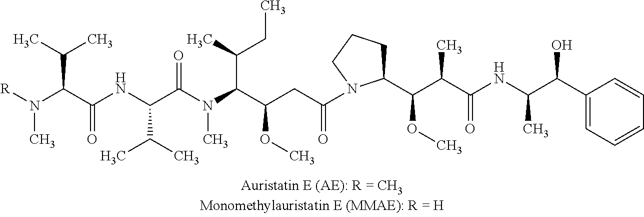

MMAE, however, possesses the disadvantage of a comparatively high systemic toxicity. For improving tumour selectivity, MMAE is used more particularly in conjunction with enzymatically cleavable valine-citrulline linkers in the ADC setting for more targeted tumour therapy [WO 2005081711-A2; S. O. Doronina et al., Bioconjugate Chem. 17, 114-124 (2006)]. Following proteolytic cleavage, MMAE is released preferably intracellularly from corresponding ADCs.

Monomethylauristatin F (MMAF) is an auristatin derivative having a C-terminal phenylalanine unit which exhibits only moderate antiproliferative activity in comparison to MMAE. This fact is very probably attributable to the free carboxyl group, whose polarity and charge adversely affect the capacity of this compound to access cells. In this connection, the methyl ester of MMAF (MMAF-OMe) has been described, as a neutral-charged prodrug derivative with cell access capability, which, in comparison to MMAF, has an in vitro cytotoxicity for various carcinoma cell lines that is increased by a number of orders of magnitude [S. O. Doronina et al., Bioconjugate Chem. 17, 114-124 (2006)]. It can be assumed that this effect is brought about by MMAF itself, which, following uptake of the prodrug into the cells, is rapidly released by intracellular ester hydrolysis.

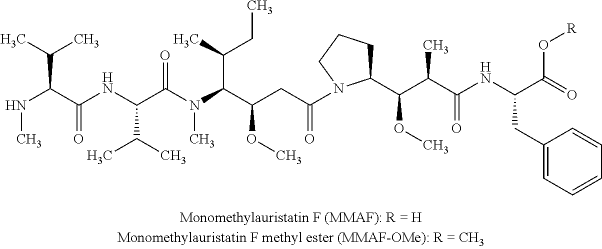

However, drug compounds based on simple ester derivatives are generally subject to the risk of chemical instability on account of non-specific ester hydrolysis, independent of the intended site of action, by means, for example, of esterases that are present in the blood plasma; this non-specific hydrolysis may significantly restrict the usefulness of such compounds in therapy.

Monomethylauristatin F (MMAF) and also various ester derivatives and amide derivatives thereof have been disclosed in WO 2005/081711-A2. Further auristatin analogues with a C-terminal, amidically substituted phenylalanine unit are described in WO 01/18032-A2. WO 02/088172-A2 and WO 2007/008603-A1 claim MMAF analogues which relate to side-chain modifications of the phenylalanine, while WO 2007/008848-A2 claims those in which the carboxyl group of the phenylalanine has been modified. Auristatin conjugates linked via the C-terminus have been recently described in WO 2009/117531-A1 [see also S. O. Doronina et al., Bioconjugate Chem. 19, 1960-1963 (2008)].

The problem addressed with the present invention was that of providing new binder-drug conjugates (ADCs) which, through combination of new N,N-dialkylauristatin derivatives with innovative, suitable linkers and binder, exhibit a very attractive activity profile, such as, for example, in terms of their specific tumour effect and/or the reduced potential of the metabolites formed intracellularly to be a substrate with respect to transporter proteins, and which are therefore suitable for the treatment and/or prophylaxis of hyperproliferative and/or angiogenic diseases, such as cancer diseases, for example.

The present invention provides binder-drug conjugates of the general formula (Ia)

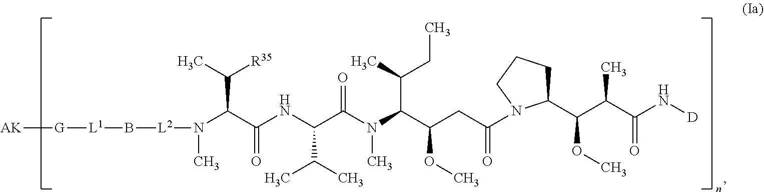

in which
n is a number from 1 to 50,
AK is a binder which binds FGFR2,
the group §-G-L¹-B-§§ is a linker,
where
§ marks the linkage site with the group AK and
§§ marks the linkage site with the nitrogen atom,
L₂ is linear (C₂-C₁₀)-alkanediyl or is a group of the formula

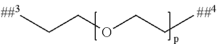

where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2-, 1,3- or 1,4-relation to one another, with inclusion of any carbon atoms situated between them, may be bridged to form a (C₃-C₆)-cycloalkyl ring or a phenyl ring,
   D is a group of the formula

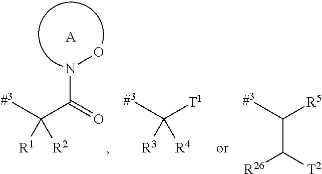

where
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
Or
R¹ and R² together with the carbon atom to which they are bonded form a (1S,2R)-2-phenylcyclopropane-1,1-diyl group of the formula

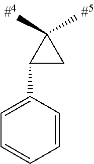

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

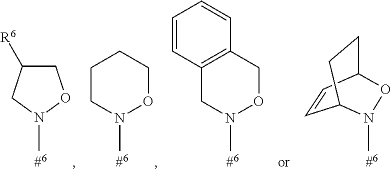

in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula

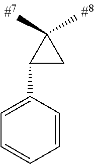

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula

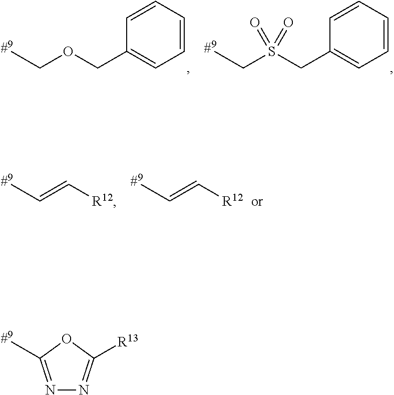

in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
   R³⁵ is methyl or hydroxy,
   and also their salts, solvates and solvates of the salts.

Compounds of the invention are the compounds of the formula (I) and their salts, solvates and solvates of the salts, the compounds of the formulae identified below and encompassed by formula (I), and their salts, solvates and solvates of the salts, and also the compounds identified below as working examples and encompassed by formula (I), and their salts, solvates and solvates of the salts, to the extent that the compounds identified below and encompassed by formula (I) are not already salts, solvates and solvates of the salts.

Depending on their structure, the compounds of the invention may exist in different stereoisomeric forms, i.e. in the form of configurational isomers or else where appropriate as conformational isomers (enantiomers and/or diastereoisomers, including those in the case of atropisomers). The present invention therefore encompasses the enantiomers and diastereomers and their respective mixtures. The stereoisomerically homogeneous constituents can be isolated from such mixtures of enantiomers and/or diastereomers in a known way; for this purpose it is preferred to use chromatographic processes, more particularly HPLC chromatography on an achiral or chiral phase.

Where the compounds of the invention can occur in tautomeric forms, the present invention encompasses all of the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number but with a different atomic mass from the atomic mass which occurs commonly or predominantly in nature. Examples of isotopes which can be incorporated into an inventive compound are those of hydrogen, carbon, nitrogen, oxygen, phosphorus, sulphur, fluorine, chlorine, bromine and iodine such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I, and ¹³¹I. Particular isotope variants of a compound of the invention, such as more particularly those in which one or more radioactive isotopes are incorporated, may be of benefit, for example, for investigating the mechanism of action or the distribution of drug in the body; owing to the comparative ease of preparation and detectability, compounds labelled with ³H or ¹⁴C isotopes are especially suitable for these purposes. Furthermore, the incorporation of isotopes, such as of deuterium, for example, may lead to certain therapeutic advantages as a consequence of greater metabolic stability of the compound, such as an extension to the half-life in the body or a reduction in the active dose required, for example; such modifications of the compounds of the invention may therefore, where appropriate, also constitute a preferred embodiment of the present invention. Isotopic variants of the compounds of the invention can be prepared by the processes known to the skilled person, as for example in accordance with the methods described later on below and the procedures reproduced in the working examples, by using corresponding isotopic modifications of the respective reagents and/or starting compounds.

Preferred salts in the context of the present invention are physiologically acceptable salts of the compounds of the invention. Also encompassed are salts which although themselves not suitable for pharmaceutical applications may nevertheless be used, for example, for isolating or purifying the compounds of the invention.

Physiologically acceptable salts of the compounds of the invention encompass acid addition salts of mineral acids, carboxylic acids and sulphonic acids, examples being salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also encompass salts of customary bases, such as, by way of example and preferably, alkali metal salts (e.g. sodium and potassium salts), alkaline earth metal salts (e.g. calcium and magnesium salts) and ammonium salts, derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylpiperidine, N-methylmorpholine, arginine, lysine and 1,2-ethylenediamine.

Solvates in the context of the invention are those forms of the compounds of the invention that form a complex in the solid or liquid state through coordination with solvent molecules. Hydrates are one specific form of solvates, in which the coordination takes place with water. Preferred solvates in the context of the present invention are hydrates.

Furthermore, the present invention also encompasses prodrugs of the compounds of the invention. The term “prodrugs” here identifies compounds which may themselves be biologically active or inactive but are converted during their residence in the body into compounds of the invention (by metabolism or hydrolysis, for example).

In the context of the present invention the definitions of the substituents, unless otherwise specified, are as follows:

(C₁-C₄)-Alkyl in the context of the invention is a linear or branched alkyl radical having 1 to 4 carbon atoms. By way of example and with preference, the following may be mentioned: methyl, ethyl, n-propyl, isopropyl, n-butyl, isobutyl, 1-methylpropyl and tert-butyl.

Alkanediyl in the context of the invention is a linear, α,ω-divalent alkyl radical having the particular number of carbon atoms indicated. By way of example and of preference, the following may be mentioned: methylene, ethane-1,2-diyl(1,2-ethylene), propane-1,3-diyl(1,3-propylene), butane-1,4-diyl(1,4-butylene), pentane-1,5-diyl(1,5-pentylene), hexane-1,6-diyl(1,6-hexylene), heptane-1,7-diyl (1,7-hexylene), octane-1,8-diyl (1,8-octylene), nonane-1,9-diyl (1,9-nonylene), decane-1,10-diyl (1,10-decylene).

(C₃-C₇)-Cycloalkyl and 3- to 7-membered carbocycle respectively in the context of the invention is a monocyclic, saturated cycloalkyl group having 3 to 7 carbon atoms. By way of example and of preference, the following may be mentioned: cyclopropyl, cyclobutyl, cyclopentyl, cyclohexyl and cycloheptyl.

The side group of an α-amino acid in the definition of R¹⁹ encompasses not only the side groups of the naturally occurring α-amino acids but also the side groups of homologues and isomers of these a-amino acids. The α-amino acid here may be in the L or D configuration or else may be present as a mixture of the L and D forms. Examples that may be given of side groups are as follows: methyl (alanine), propan-2-yl (valine), propan-1-yl (norvaline), 2-methylpropan-1-yl (leucine), 1-methylpropan-1-yl (isoleucine), butan-1-yl (norleucine), tert-butyl (2-tert-butylglycine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-yl (lysine), 4-amino-3-hydroxybutan-1-yl (hydroxylysine), 3-aminopropan-1-yl (ornithine), 2-aminoethyl (2,4-diaminobutyric acid), aminomethyl (2,3-diaminopropionic acid), 3-guanidinopropan-1-yl (arginine), 3-ureidopropan-1-yl (citrulline). Preferred α-amino acid side groups in the definition of R¹⁹ are methyl (alanine), propan-2-yl (valine), 2-methylpropan-1-yl (leucine), benzyl (phenylalanine), imidazol-4-ylmethyl (histidine), hydroxymethyl (serine), 1-hydroxyethyl (threonine), 4-aminobutan-1-yl (lysine), 3-aminopropan-1-yl (ornithine), 2-aminoethyl (2,4-diaminobutyric acid), aminomethyl (2,3-diaminopropionic acid), 3-guanidinopropan-1-yl (arginine). The L configuration is preferred in each case.

A 4- to 7-membered heterocycle in the context of the invention is a monocyclic, saturated heterocycle having a total of 4 to 7 ring atoms, which contains one or two ring heteroatoms from the series N, O, S, SO and/or SO₂ and is linked via a ring carbon atom or optionally a ring nitrogen atom. Preference is given to a 5- to 7-membered heterocycle having one or two ring heteroatoms from the series N, O and/or S, more preferably a 5- or 6-membered heterocycle having one or two ring heteroatoms from the series N and/or O. By way of example, the following may be mentioned: azetidinyl, oxetanyl, pyrrolidinyl, pyrazolidinyl, tetrahydrofuranyl, thiolanyl, piperidinyl, piperazinyl, tetrahydropyranyl, tetrahydrothiopyranyl, morpholinyl, thiomorpholinyl, hexahydroazepinyl and hexahydro-1,4-diazepinyl. Preference is given to pyrrolidinyl, tetrahydrofuranyl, piperidinyl, piperazinyl, tetrahydropyranyl and morpholinyl.

In the formula of the group which may be represented by A, B, D, G, L¹, L², L⁴, R¹, R², R³, R⁴ and R⁵, respectively, the end point of the line at which the symbol #⁶, *, **, #³, #¹, #², ##¹, #², ##³, ##⁴, ***, ****, #⁴, #⁵, #⁶, #⁷, #⁸ or #⁹ is located is not a carbon atom or a CH₂ group, but instead is part of the bond to the atom designated in each case, to which the A, B, D, G, L¹, L², L⁴, R¹, R², R³, R⁴ or R⁵ is bonded.

In the context of the present invention, all radicals which occur multiply have their definition independently of one another. If radicals in the compounds of the invention are substituted, the radicals, unless otherwise specified, may be substituted one or more times. Substitution by one or by two identical or different substituent(s) is preferred. Particularly preferred is substitution by one substituent.

In the context of the present invention the terms used, unless otherwise specified, have the following definitions:

The term “linker” is understood in the broadest sense as a chemical unit which comprises a covalent bond or a series of atoms that links a binder covalently to a drug. The term “linker” is understood preferably as a series of atoms in the sense of the present invention that links a binder covalently to a drug. Furthermore, linkers may be represented, for example, by divalent chemical units, such as alkyldiyls, aryldiyls, heteroaryldiyls, heterocyclyldiyls, dicarbonyl acid esters, dicarbonyl acid amides.

The term “binder” is understood in the broadest sense as a molecule which binds to a target molecule which is present on a particular target cell population to be addressed with the binder-drug conjugate. The term “binder” should be understood in its broadest interpretation and encompasses, for example, lectins, proteins which are able to bind particular sugar chains, or phospholipid-binding proteins. Such binders comprise, for example, high molecular mass proteins (binding proteins), polypeptides or peptides (binding peptides), non-peptidic (e.g. aptamers (U.S. Pat. No. 5,270,163) review article by Keefe A D., et al., Nat. Rev. Drug Discov. 2010; 9:537-550), or vitamins) and all other cell-binding molecules or substances. Binding proteins are, for example, antibodies and antibody fragments or antibody mimetics such as, for example, affibodies, adnectins, anticalins, DARPins, avimers, nanobodies (review articles by Gebauer M. et al., Curr. Opinion in Chem. Biol. 2009; 13:245-255; Nuttall S. D. et al., Curr. Opinion in Pharmacology 2008; 8:608-617). Binding peptides are, for example, ligands of a ligand-receptor pair, such as VEGF in the ligand-receptor pair VEGF/KDR, such as transferrin of the ligand-receptor pair transferrin/transferrin receptor, or cytokines/cytokine receptor, such as TNFalpha in the ligand receptor pair TNFalpha/TNFalpha receptor.

The term “epitope” as used herein encompasses any determinants of a protein that are able to bind specifically to an immunoglobulin or T-cell receptor. Such determinants commonly consist of chemically active surface arrangements of molecules, such as amino acids, carbohydrates or a combination thereof, for example, which commonly have a specific three-dimensional structure and also defined charge properties. Two antibodies bind to the same epitope if it is shown in a competitive binding assay format that the first antibody competes with the second antibody. Binding assays of this kind are known to the skilled person.

A “target molecule” is understood in the broadest sense to be a molecule which is present in the target cell population, and may be a protein (e.g. a receptor of a growth factor) or a non-peptidic molecule (e.g. a sugar or phospholipid). Preferably it is a receptor or an antigen.

The term “extracellular” target molecule describes a target molecule which is attached to the cell and which is located on the outside of a cell or the part of a target molecule which is located on the outside of a cell, i.e. a binder may bind to an intact cell at its extracellular target molecule. An extracellular target molecule may be anchored in the cell membrane or may be part of the cell membrane. The skilled person knows of methods for identifying extracellular target molecules. For proteins this may be done via determination of the transmembrane domain(s) and the orientation of the protein in the membrane. This data is generally recorded in protein databases (e.g. SwissProt).

The term “cancer target molecule” describes a target molecule which is multiply present on one or more cancer cell types in comparison to non-cancer cells of the same tissue type. The cancer target molecule is preferably present selectively on one or more cancer cell types in comparison to non-cancer cells of the same tissue type, with “selectively” describing an at least twofold accumulation on cancer cells in comparison to non-cancer cells of the same tissue type (a “selective cancer target molecule”). The use of cancer target molecules allows selective therapy of cancer cells with the conjugates of the invention.

The binder may be linked via a bond to the linker. The linking of the binder may take place by means of a heteroatom of the binder. Inventive heteroatoms of the binder that may be used for linking are sulphur (in one embodiment via a sulphhydryl group of the binder), oxygen (in accordance with the invention by means of a carboxyl or hydroxy group of the binder) and nitrogen (in one embodiment via a primary or secondary amine group or amide group of the binder). These heteroatoms may be present in the natural binder or may be introduced by means of methods of chemistry or molecular biology. In accordance with the invention, the linking of the binder to the toxophore has little influence over the binding activity of the binder to the target molecule. In a preferred embodiment the linking has no influence on the binding activity of the binder to the target molecule.

The term “antibody” is understood in accordance with the present invention in its broadest sense and encompasses immunoglobulin molecules, examples being intact or modified monoclonal antibodies, polyclonal antibodies or multispecific antibodies (e.g. bispecific antibodies). An immunoglobulin molecule preferably comprises a molecule having four polypeptide chains, two heavy chains (H chains) and two light chains (L chains), which are linked typically by disulphide bridges. Each heavy chain comprises a variable domain of the heavy chain (abbreviated to VH) and a constant domain of the heavy chain. The constant domain of the heavy chain may encompass, for example, three domains CH1, CH2 and CH3. Each light chain comprises a variable domain (abbreviated to VL) and a constant domain. The constant domain of the light chain comprises one domain (abbreviated to CL). The VH and VL domains may be further subdivided into regions having hypervariability, also called complementarity-determining regions (abbreviated to CDR), and regions having a low sequence variability (“framework region”, abbreviated to FR). Each VH and VL region is typically composed of three CDRs and up to four FRs. For example, in the following order from the amino terminus to the carboxy terminus: FR1, CDR1, FR2, CDR2, FR3, CDR3, FR4. An antibody may be obtained from any species suitable for the antibody, such as, for example, rabbit, llama, camel, mouse or rat. In one embodiment the antibody is of human or murine origin. An antibody may for example be human, humanized or chimeric.

The term “monoclonal” antibody identifies antibodies which have been obtained from a population of substantially homogeneous antibodies, i.e. individual antibodies of the population are identical except for naturally occurring mutations which may occur in small numbers. Monoclonal antibodies recognize a single antigenic binding site with a high specificity. The term “monoclonal antibody” does not refer to a particular production method.

The term “intact” antibody refers to antibodies which comprise not only an antigen-binding domain but also the constant domain of the light and heavy chain. The constant domain may be a naturally occurring domain, or a variant thereof in which a plurality of amino acid positions have been altered.

The term “modified intact” antibody refers to intact antibodies which have been fused with another polypeptide or protein, not originating from an antibody, via the amino terminus or carboxyl terminus thereof, by means of a covalent bond (e.g. a peptide linkage). Furthermore, antibodies may be modified by introducing reactive cysteines at defined locations, in order to facilitate coupling to a toxophore (see Junutula et al. Nat Biotechnol. 2008 August; 26(8):925-32).

The term “human” antibody identifies antibodies which can be obtained from a human being or are synthetic human antibodies. A “synthetic” human antibody is an antibody which in parts or as a whole is obtainable from synthetic sequences in silico which are based on the analysis of human antibody sequences. A human antibody may be encoded, for example, by a nucleic acid which has been isolated from a library of antibody sequences which are of human origin. One example of such antibodies can be found in Söderlind et al., Nature Biotech. 2000, 18:853-856.

The term “humanized” or “chimeric” antibody describes antibodies which consist of a non-human and of a human sequence component. In these antibodies, part of the sequences of the human immunoglobulin (recipient) is replaced by sequence components of a non-human immunoglobulin (donor). In many cases the donor is a murine immunoglobulin. With humanized antibodies, amino acids of the CDR in the recipient are replaced by amino acids of the donor. In some cases, amino acids of the framework as well are replaced by corresponding amino acids of the donor. In some cases the humanized antibody contains amino acids which were present neither in the recipient nor in the donor and which were inserted during the optimization of the antibody. In the case of chimeric antibodies, the variable domains of the donor immunoglobulin are fused with the constant regions of a human antibody.

The term complementarity-determining region (CDR) as used here refers to those amino acids in a variable antibody domain that are necessary for binding to the antigen. Every variable region typically has three CDR regions, identified as CDR1, CDR2 and CDR3. Each CDR region may comprise amino acids according to the definition of Kabat and/or amino acids of a hypervariable loop, defined according to Chotia. The definition according to Kabat encompasses, for example, the region of approximately amino acid position 24-34 (CDR1), 50-56 (CDR2) and 89-97 (CDR3) of the variable light chain and 31-35 (CDR1), 50-65 (CDR2) and 95-102 (CDR3) of the variable heavy chain (Kabat et al., Sequences of Proteins of Immulological Interest, 5th Ed. Public Health Service, National Institutes of Health, Bethesda, Md. (1991)). The definition according to Chotia encompasses, for example, the region of approximately amino acid position 26-32 (CDR1), 50-52 (CDR2) and 91-96 (CDR3) of the variable light chain and 26-32 (CDR1), 53-55 (CDR2) and 96-101 (CDR3) of the variable heavy chain Chothia and Lesk; J Mol Biol 196: 901-917 (1987)). In some cases a CDR may comprise amino acids from one CDR region as defined by Kabat and Chotia.

Depending on the amino acid sequence of the constant domain of the heavy chain, antibodies may be divided into different classes. There are five main classes of intact antibodies: IgA, IgD, IgE, IgG and IgM, and a number of them may be broken down into further subclasses (isotypes), e.g. IgG1, IgG2, IgG3, IgG4, IgA1 and IgA2. The constant domains of the heavy chain that correspond to the different classes are identified as [alpha/α], [delta/δ], [epsilon/ε], [gamma/γ] and [mu/μ]. Both the three-dimensional structure and the subunit structure of antibodies are known.

The term “functional fragment” or “antigen-binding antibody fragments” of a antibodyimmunoglobulin is defined as a fragment of an antibodyimmunoglobulin (e.g. the variable domains of an IgG) which further encompasses the antigen binding domains of the antibodyimmunoglobulin. The “antigen binding domain” of an antibody typically encompasses one or more hypervariable regions of an antibody, e.g. the CDR, CDR2 and/or CDR3 region. However, the “framework” or “scaffold” region of an antibody may also play a part with regard to the binding of the antibody to the antigen. The framework region forms the scaffold for the CDRs. The antigen-binding domain preferably encompasses at least amino acids 4 to 103 of the variable light chain and amino acid 5 to 109 of the variable heavy chain, more preferably amino acid 3 to 107 of the variable light chain and 4 to 111 of the variable heavy chain, particular preference being given to the complete variable light and heavy chains, i.e. amino acid 1-109 of the VL and 1 to 113 of the VH (numbering according to WO9708320).

“Functional fragments” or “antigen-binding antibody fragments” of the invention encompass, non-conclusively, Fab, Fab′, F(ab′)₂ and Fv fragments, diabodies, Single Domain Antibodies (DAbs), linear antibodies, individual chains of antibodies (single-chain Fv, abbreviated to ScFv); and multispecific antibodies, such as bi and tri-specific antibodies, for example, formed from antibody fragments C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). Antibodies other than “multispecific” or “multifunctional” antibodies are those having identical binding sites. Multispecific antibodies may be specific for different epitopes of an antigen or may be specific for epitopes of more than one antigen (see, for example WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; or Kostelny et al., 1992, J. Immunol. 148: 1547 1553). An F(ab′)₂ or Fab molecule may be constructed such that the number of intermolecular disulphide interactions occurring between the Ch1 and the CL domains can be reduced or else completely prevented.

“Functional fragments” or “antigen-binding antibody fragments” may be fused with another polypeptide or protein, not originating from an antibody, via the amino terminus or carboxyl terminus thereof, by means of a covalent bond (e.g. a peptide linkage). Furthermore, antibodies and antigen-binding fragments may be modified by introducing reactive cysteines at defined locations, in order to facilitate coupling to a toxophore (see Junutula et al. Nat Biotechnol. 2008 August; 26(8):925-32).

Polyclonal antibodies can be prepared by methods known to a person of ordinary skill in the art. Monoclonal antibodies may be prepared by methods known to a person of ordinary skill in the art (Köhler and Milstein, Nature, 256, 495-497, 1975). Human and humanized monoclonal antibodies may be prepared by methods known to a person of ordinary skill in the art (Olsson et al., Meth Enzymol. 92, 3-16 or Cabilly et al U.S. Pat. No. 4,816,567 or Boss et al U.S. Pat. No. 4,816,397).

A person of ordinary skill in the art is aware of diverse methods for preparing human antibodies and fragments thereof, such as, for example, by means of transgenic mice (N Lonberg and D Huszar, Int Rev Immunol. 1995; 13(1):65-93) or Phage Display Technologies (Clackson et al., Nature. 1991 Aug. 15; 352(6336):624-8). Antibodies of the invention may be obtained from recombinant antibody libraries consisting for example of the amino acid sequences of a multiplicity of antibodies compiled from a large number of healthy volunteers. Antibodies may also be produced by means of known recombinant DNA technologies. The nucleic acid sequence of an antibody can be obtained by routine sequencing or is available from publically accessible databases.

An “isolated” antibody or binder has been purified to remove other constituents of the cell. Contaminating constituents of a cell which may interfere with a diagnostic or therapeutic use are, for example, enzymes, hormones, or other peptidic or non-peptidic constituents of the cell. A preferred antibody or binder is one which has been purified to an extent of more than 95%, relative to the antibody or binder (determined for example by Lowry method, UV-Vis spectroscopy or by SDS capillary gel electrophoresis). Furthermore an antibody which has been purified to such an extent that it is possible to determine at least 15 amino acids of the amino terminus or of an internal amino acid sequence, or which has been purified to homogeneity, the homogeneity being determined by SDS-PAGE under reducing or non-reducing conditions (detection may be determined by means of Coomassie Blau staining or preferably by silver coloration). However, an antibody is normally prepared by one or more purification steps.

The term “specific binding” or “binds specifically” refers to an antibody or binder which binds to a predetermined antigentarget molecule. Specific binding of an antibody or binder typically describes an antibody or binder having an affinity of at least 10⁻⁷ M, with the antibody or binder having an at least two times higher affinity for the predetermined antigentarget molecule than for a non-specific antigen/target molecule (e.g. bovine serum albumin, or casein) which is not the predetermined antigen/target molecule or a closely related antigentarget molecule.

Antibodies which are specific against a cancer cell antigen can be prepared by a person of ordinary skill in the art by means of methods with which he or she is familiar (such as recombinant expression, for example) or may be acquired commercially (as for example from Merck KGaA, Germany). Examples of known commercially available antibodies in cancer therapy are Erbitux® (cetuximab, Merck KGaA), Avastin® (bevacizumab, Roche) and Herceptin® (trastuzumab, Genentech). Trastuzumab is a recombinant humanized monoclonal antibody of the IgG1kappa type which in a cell-based assay (Kd=5 nM) binds the extracellular domains of the human epidermal growth receptor with high affinity. The antibody is produced recombinantly in CHO cells.

A preferred subject of the invention are binder-drug conjugates of the general formula (Ia) in which

n is a number from 1 to 50,

### AK is AK₁ or AK₂

where
AK₁ is a binder which binds FGFR2 and is bonded via a sulphur atom of the binder to the group G,
AK₂ is a binder which binds FGFR2 and is bonded via a nitrogen atom of the binder to the group G,
   G when AK=AK₁, is a group of the formula

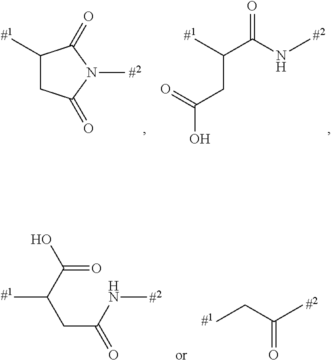

where
#¹ marks the linkage site with the sulphur atom of the binder,
#² marks the linkage site with the group L¹,
or
when AK=AK₂, is carbonyl,
   L¹ is a bond, linear (C₁-C₁₀)-alkanediyl, a group of the formula

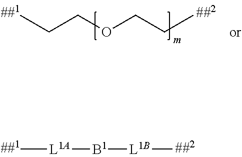

where
m is a number from 2 to 6,
##¹ marks the linkage site with the group G,
##² marks the linkage site with the group B,
L^{1A} is linear (C₂-C₁₀)-alkanediyl,
B¹ is a group of the formula

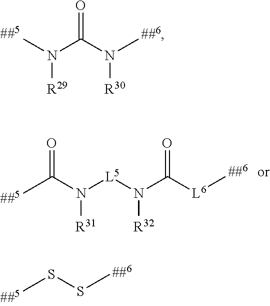

in which
##⁵ marks the linkage site with the group L^{1A},
##⁶ marks the linkage site with the group L^{1B},
L⁵ is a bond or (C₂-C₄)-alkanediyl,
L⁶ is a bond or a group of the formula

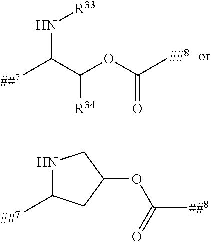

in which
##⁷ marks the linkage site with the carbonyl group,
##⁸ marks the linkage site with L^{1B},
R³³ is hydrogen, (C₁-C₄)-alkylcarbonyl, tert-butyloxycarbonyl or benzyloxycarbonyl,
R³⁴ is hydrogen or methyl,
R²⁹ is hydrogen or (C₁-C₄)-alkyl,
R³⁰ is hydrogen or (C₁-C₄)-alkyl,
or
R²⁹ and R³⁰ together with the atoms to which they are bonded form a 5- or 6-membered heterocycle,
R³¹ is hydrogen or (C₁-C₄)-alkyl,
R³² is hydrogen or (C₁-C₄)-alkyl,
or
R³¹ and R³² together with the atoms to which they are bonded form a 5- or 6-membered heterocycle,
L^{1B} is linear (C₂-C₁₀)-alkanediyl,
   and
   where (C₁-C₁₀)-alkanediyl may be substituted by 1 to 4 substituents selected independently of one another from the group consisting of methyl, hydroxy and benzyl,
   and
   where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
      B is a bond or a group of the formula

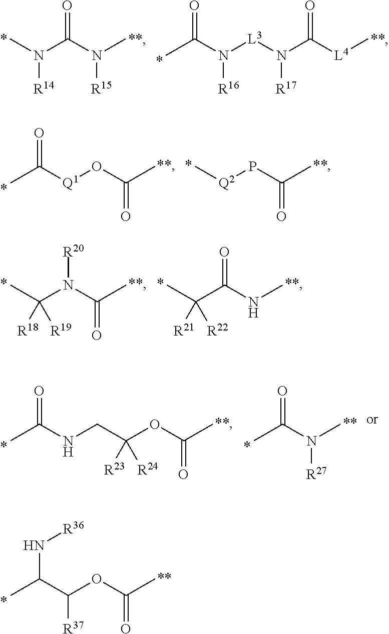

where
* marks the linkage site with L¹,
** marks the linkage site with L²,
P is O or NH,
L³ is a bond or (C₂-C₄)-alkanediyl,
L⁴ is a bond or a group of the formula

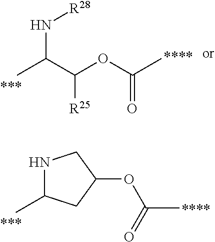

in which
*** marks the linkage site with the carbonyl group,
**** marks the linkage site with L²,
R²⁵ is hydrogen or methyl,
R²⁸ is hydrogen, (C₁-C₄)-alkylcarbonyl, tert-butyloxycarbonyl or benzyloxycarbonyl,
Q¹ is a 4- to 7-membered heterocycle,
Q² is a 3- to 7-membered carbocycle or a 4- to 7-membered heterocycle,
R¹⁴ is hydrogen or (C₁-C₄)-alkyl,
R¹⁵ is hydrogen or (C₁-C₄)-alkyl,
or
R¹⁴ and R¹⁵ together with the atoms to which they are bonded form a 5- or 6-membered heterocycle,
R¹⁶ is hydrogen or (C₁-C₄)-alkyl,
R¹⁷ is hydrogen or (C₁-C₄)-alkyl,
or
R¹⁶ and R¹⁷ together with the atoms to which they are bonded form a 5- or 6-membered heterocycle,
R¹⁸ is hydrogen or (C₁-C₄)-alkyl,
R¹⁹ is hydrogen or the side group of a natural α-amino acid or of its homologues or isomers,
R²⁰ is hydrogen or (C₁-C₄)-alkyl,
or
R¹⁹ and R²⁰ together with the atoms to which they are bonded form a pyrrolidinyl ring,
R²¹ is hydrogen or (C₁-C₄)-alkyl,
R²² is hydrogen or (C₁-C₄)-alkyl,
or
R²¹ and R²² together with the atoms to which they are bonded form a 3- to 7-membered carbocycle,
R²³ is (C₁-C₄)-alkyl,
R²⁴ is hydrogen or (C₁-C₄)-alkyl,
R²⁷ is hydrogen or (C₁-C₄)-alkyl,
R³⁶ is hydrogen, (C₁-C₄)-alkylcarbonyl, tert-butyloxycarbonyl or benzyloxycarbonyl,
R³⁷ is hydrogen or methyl,
or
R³⁶ and R³⁷ together with the atoms to which they are bonded form a pyrrolidine ring,
   L² is linear (C₂-C₁₀)-alkanediyl or is a group of the formula

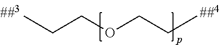

where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
   D is a group of the formula

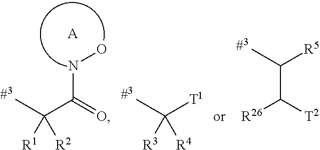

in which
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
   or
R¹ and R² together with the carbon atom to which they are bonded form a (1S,2R)-2-phenylcyclopropane-1,1-diyl group of the formula

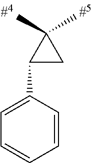

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

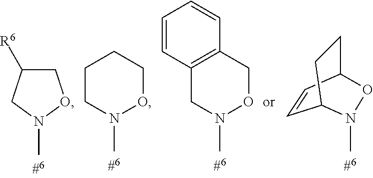

in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula

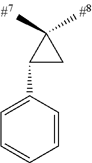

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   or
   R⁸ and R⁹ together with the nitrogen atom to which they are bonded form a 4- to 7-membered heterocycle,
   R¹⁰ is benzoyl,
   R¹¹ is benzyl, which may be substituted in the phenyl group by methoxycarbonyl or carboxyl,
R⁵ is hydrogen, methyl or a group of the formula

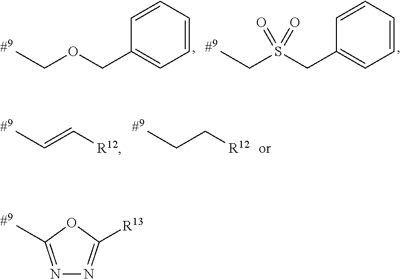

in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
   R³⁵ is methyl or hydroxy,
   and also their salts, solvates and solvates of the salts.
   Preferred subject of the invention are binder-drug conjugates of the general formula (Ia) in which
   n is a number from 1 to 20,

### AK is AK₁ or AK₂

where
AK₁ is a binder which binds FGFR2 and is bonded via the sulphur atom of a cysteine residue of the binder to the group G,
AK₂ is a binder which binds FGFR2 and is bonded via the NH side group of a lysine residue of the binder to the group G,
   G when AK=AK₁, is a group of the formula

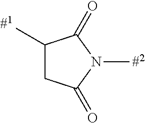

in which
#¹ marks the linkage site with the cysteine residue of the binder,
#² marks the linkage site with the group L¹,
or
when AK=AK₂, is carbonyl,
   L¹ is a bond, linear (C₂-C₆)-alkanediyl, a group of the formula

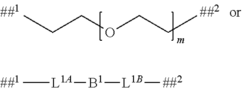

where
m is a number from 2 to 6,
##¹ marks the linkage site with the group G,
##² marks the linkage site with the group B,
L^{1A} is linear (C₂-C₆)-alkanediyl,
B¹ is a group of the formula

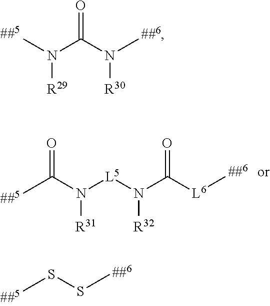

in which
##⁵ marks the linkage site with the group L^{1A},
##⁶ marks the linkage site with the group L^{1B},
L⁵ is a bond,
L⁶ is a bond or a group of the formula

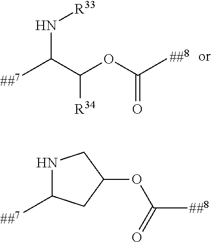

in which
##⁷ marks the linkage site with the carbonyl group,
##⁸ marks the linkage site with L^{1B},
R³³ is hydrogen, methylcarbonyl or tert-butyloxycarbonyl,
R³⁴ is hydrogen or methyl,
R²⁹ is hydrogen,
R³⁰ is hydrogen,
R³¹ is hydrogen or methyl,
R³² is hydrogen or methyl,
L^{1B} is linear (C₂-C₆)-alkanediyl,
and
where (C₂-C₆)-alkanediyl may be substituted by 1 or 2 methyl substituents,
   B is a bond or a group of the formula

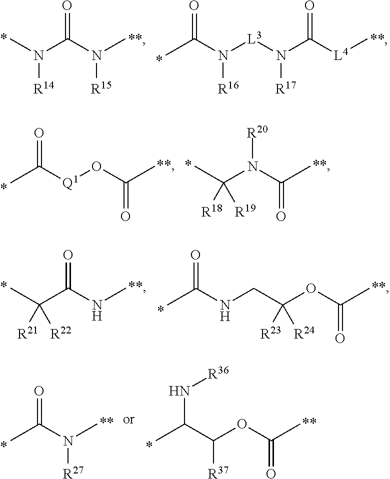

where
* marks the linkage site with L¹,
** marks the linkage site with L²,
L³ is a bond or ethane-1,2-diyl,
L⁴ is a bond or a group of the formula

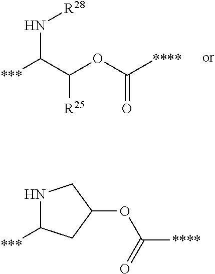

in which
*** marks the linkage site with the carbonyl group,
**** marks the linkage site with L²,
R²⁵ is hydrogen or methyl,
R²⁸ is hydrogen, methylcarbonyl or tert-butyloxycarbonyl,
Q¹ is a 4- to 7-membered heterocycle,
R¹⁴ is hydrogen,
R¹⁵ is hydrogen,
R¹⁶ is hydrogen or methyl,
R¹⁷ is hydrogen or methyl,
or
R¹⁶ and R¹⁷ together with the atoms to which they are bonded form a piperazinyl ring,
R¹⁸ is hydrogen,
R¹⁹ is hydrogen, methyl, propan-2-yl, 2-methylpropan-1-yl or 1-methylpropan-1-yl,
R²⁰ is hydrogen or methyl,
or
R¹⁹ and R²⁰ together with the atoms to which they are bonded form a pyrrolidinyl ring,
R²¹ is hydrogen or methyl,
R²² is hydrogen or methyl,
or
R²¹ and R²² together with the atoms to which they are bonded form a cyclopropyl ring,
R²³ is methyl,
R²⁴ is hydrogen or methyl,
R²⁷ is hydrogen,
R³⁶ is hydrogen, methylcarbonyl or tert-butyloxycarbonyl,
R³⁷ is hydrogen or methyl,
or
R³⁶ and R³⁷ together with the atoms to which they are bonded form a pyrrolidine ring,
L² is linear (C₂-C₆)-alkanediyl or is a group of the formula

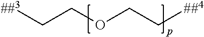

where
p is a number from 2 to 6,
##³ marks the linkage site with the group B,
##⁴ marks the linkage site with the nitrogen atom,
where (C₂-C₁₀)-alkanediyl may be substituted by 1 or 2 methyl substituents,
   D is a group of the formula

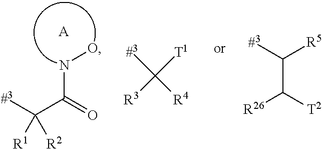

where
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula

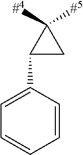

in which
#⁴ marks the linkage site with the adjacent nitrogen atom,
#⁵ marks the linkage site with the carbonyl group,
the ring A with the N—O moiety present therein is a mono- or bicyclic, optionally

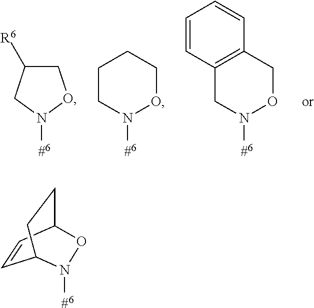

in which
#⁶ marks the linkage site with the carbonyl group,
R⁶ is hydrogen, hydroxy or benzyloxy,
R³ is hydrogen,
R⁴ is 1-hydroxyethyl, benzyl, 4-hydroxybenzyl, 1-phenylethyl or 1H-indol-3-ylmethyl,
or
R³ and R⁴ together with the carbon atom to which they are bonded form a (1S,2R)-2-phenylcyclopropane-1,1-diyl group of the formula

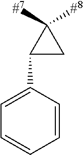

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula

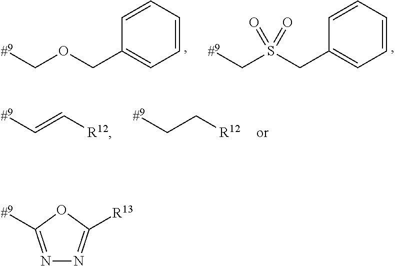

in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
   R³⁵ is methyl or hydroxy,
   and also their salts, solvates and solvates of the salts.
   Preferred subject matter of the invention are binder-drug conjugates of the general formula (Ia), in which
   n is a number from 1 to 10,

### AK is AK₁ or AK₂

where
AK₁ is a binder which binds FGFR2 and is bonded via the sulphur atom of a cysteine residue of the binder to the group G,
AK₂ is a binder which binds FGFR2 and is bonded via the NH side group of a lysine residue of the binder to the group G,
   G when AK=AK₁, is a group of the formula

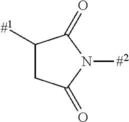

in which
#¹ marks the linkage site with the cysteine residue of the binder,
#2 marks the linkage site with the group L¹,
or
when AK=AK₂, is carbonyl,
   L¹ is a bond, linear (C₂-C₆)-alkanediyl, a group of the formula

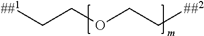

where
m is a number 2 or 3,
##¹ marks the linkage site with the group G,
##² marks the linkage site with the group B,
where (C₂-C₆)-alkanediyl may be substituted by 1 or 2 methyl substituents,
   B is a bond or a group of the formula

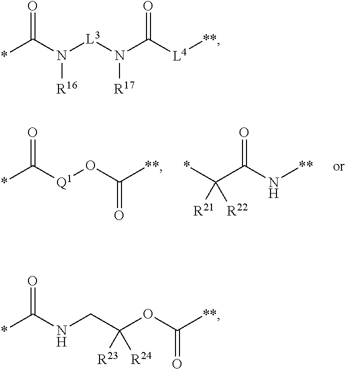

where
* marks the linkage site with L¹,
** marks the linkage site with L²,
L³ is a bond or ethane-1,2-diyl,
L⁴ is a bond or a group of the formula

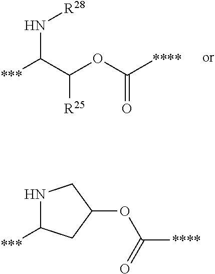

in which
*** marks the linkage site with the carbonyl group,
**** marks the linkage site with L²,
R²⁵ is methyl,
R²⁸ is hydrogen, methylcarbonyl or tert-butyloxycarbonyl,
Q¹ is piperidine-1,4-diyl,
R¹⁶ is hydrogen or methyl,
R¹⁷ is hydrogen or methyl,
or
R¹⁶ and R¹⁷ together with the atoms to which they are bonded form a piperazinyl ring,
R²¹ is hydrogen or methyl,
R²² is hydrogen or methyl,
or
R²¹ and R²² together with the atoms to which they are bonded form a cyclopropyl ring,
R²³ is methyl,
R²⁴ is hydrogen,
   L² is linear (C₂-C₆)-alkanediyl or is a group of the formula

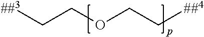

where
p is a number from 2 to 6,
##³ marks the linkage site with the group B,
##⁴ marks the linkage site with the nitrogen atom,
   D is a group of the formula

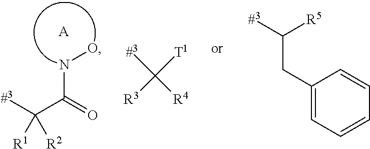

where
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl, or
R¹ and R² together with the carbon atom to which they are bonded form a (1S,2R)-2-phenylcyclopropane-1,1-diyl group of the formula

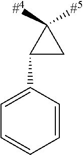

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

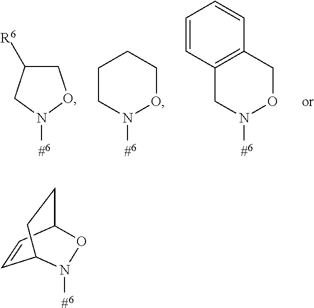

in which
#⁶ marks the linkage site with the carbonyl group,
R⁶ is hydrogen, hydroxy or benzyloxy,
R³ is hydrogen,
R⁴ is benzyl, 4-hydroxybenzyl, 1-phenylethyl or 1H-indol-3-ylmethyl,
or
R³ and R⁴ together with the carbon atom to which they are bonded form a (1S,2R)-2-phenylcyclopropane-1,1-diyl group of the formula

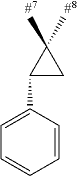

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   R¹¹ is benzyl, which may be substituted in the phenyl group by methoxycarbonyl or carboxyl,
R⁵ is hydrogen, methyl or a group of the formula

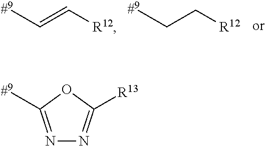

in which
#⁹ marks the linkage site with —CHCH₂phenyl,
R¹² is phenyl which may be substituted by methoxycarbonyl, carboxyl or a group of the formula —S(O)₂OH,
R¹³ is phenyl which may be substituted by methoxycarbonyl or carboxyl,
   R³⁵ is methyl or hydroxy,
   and also their salts, solvates and solvates of the salts.
   Preferred subject matter of the present invention are binder-drug conjugates of the general formula (Ia), in which
   n is a number from 1 to 10,

### AK is AK₂,

where
AK₂ is a binder which binds FGFR2 and is bonded via the NH side group of a lysine residue of the binder to the group G,
   G is carbonyl,
   L¹ is a bond,
   B is a bond,
   L² is linear (C₃-C₆)-alkanediyl or is a group of the formula

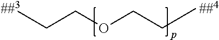

where
P is a number 2 or 3,
##³ marks the linkage site with the group B,
##⁴ marks the linkage site with the nitrogen atom,
   D is a group of the formula

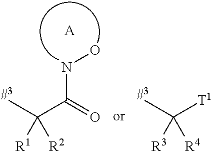

where
#³ marks the linkage site with the nitrogen atom,
R¹ is hydrogen,
R² is benzyl, 4-hydroxybenzyl or 1H-indol-3-ylmethyl,
or
R¹ and R² together with the carbon atom to which they are bonded form a (1S,2R)-2-phenylcyclopropane-1,1-diyl group of the formula

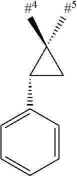

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

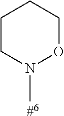

in which
#⁶ marks the linkage site with the carbonyl group,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula

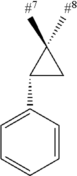

in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹
   in which
   R⁷ is hydrogen, methyl, ethyl, n-propyl, tert-butyl, benzyl or adamantylmethyl,
   R⁸ is hydrogen,
   R⁹ is hydrogen or benzyl,
      R³⁵ is methyl,
      and also their salts, solvates and solvates of the salts.
      Preferred subject matter of the invention are binder-drug conjugates of the general formula (Ia), in which
      n is a number from 1 to 10,

### AK is AK₁,

where
AK₁ is a binder which binds FGFR2 and is bonded via the sulphur atom of a cysteine residue of the binder to the group G,
   G is a group of the formula

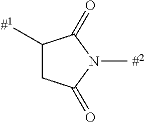

where
#¹ marks the linkage site with the cysteine residue of the binder,
#² marks the linkage site with the group L¹,
   L¹ is a bond, linear (C₃-C₅)-alkanediyl or a group of the formula

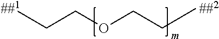

where
m is a number 2 or 3,
##¹ marks the linkage site with the group G,
##² marks the linkage site with the group B,
where (C₃-C₅)-alkanediyl may be substituted by 1 or 2 methyl substituents,
   B is a bond or a group of the formula

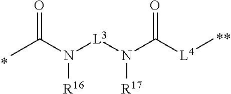

where
* marks the linkage site with L¹,
** marks the linkage site with L²,
L³ is a bond or ethane-1,2-diyl,
L⁴ is a bond or a group of the formula

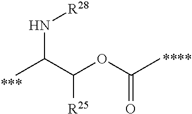

in which
*** marks the linkage site with the carbonyl group,
**** marks the linkage site with L²,
R²⁵ is methyl,
R²⁸ is hydrogen, methylcarbonyl or tert-butyloxycarbonyl,
R¹⁶ is hydrogen or methyl,
R¹⁷ is hydrogen or methyl,
or
R¹⁶ and R¹⁷ together with the atoms to which they are bonded form a piperazinyl ring,
   L² is linear (C₃-C₅)-alkanediyl or is a group of the formula

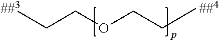

where
p is a number 2 or 3,
##³ marks the linkage site with the group B,
##⁴ marks the linkage site with the nitrogen atom,
   D is a group of the formula

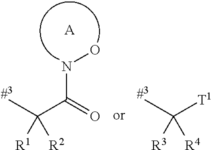

where
#³ marks the linkage site with the nitrogen atom,
R¹ is hydrogen,
R² is benzyl, 4-hydroxybenzyl or 1H-indol-3-ylmethyl,
or
R¹ and R² together with the carbon atom to which they are bonded form a (1S,2R)-2-phenylcyclopropane-1,1-diyl group of the formula

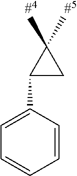

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

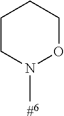

in which
#⁶ marks the linkage site with the carbonyl group,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula

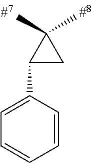

in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹,
   in which
   R⁷ is hydrogen, methyl, ethyl, n-propyl, tert-butyl, benzyl or adamantylmethyl,
   R⁸ is hydrogen,
   R⁹ is hydrogen or benzyl,
      R³⁵ is methyl,
      and also their salts, solvates and solvates of the salts.

Additionally preferably provided by the present invention is a binder-drug conjugate as described above, where the binder comprises the amino acid sequence of the variable light and heavy chains of the antibody M048-D01-hIgG1-b, reproduced in SEQ ID NO: 14 (V1) and SEQ ID NO: 13 (Vh),

the amino acid sequence of the light and heavy chain of the antibody M048-D01-hIgG1-b reproduced in SEQ ID NO: 9 (light chain) and SEQ ID NO: 10 (heavy chain).

Additionally provided by the present invention are compounds of the formula (XXXa)

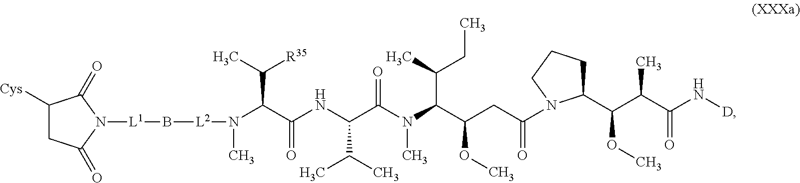

in which
Cys is a cysteine residue which is bonded via the sulphur atom of the side chain to a carbon atom of the succinimide,
   L¹ is a bond, linear (C₁-C₁₀)-alkanediyl, a group of the formula

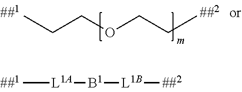

where
m is a number from 2 to 6,
##¹ marks the linkage site with the group G,
##² marks the linkage site with the group B,
L^{1a} is linear (C₂-C₁₀)-alkanediyl,
B¹ is a group of the formula

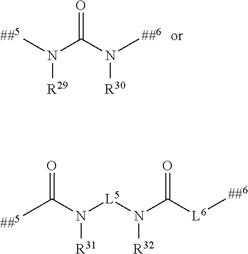

in which
##⁵ marks the linkage site with the group L^{1A},
##⁶ marks the linkage site with the group L^{1B},
L⁵ is a bond or (C₂-C₄)-alkanediyl,
L⁶ is a bond,
R²⁹ is hydrogen or (C₁-C₄)-alkyl,
R³⁰ is hydrogen or (C₁-C₄)-alkyl,
or
R²⁹ and R³⁰ together with the atoms to which they are bonded form a 5- or 6-membered heterocycle,
R³¹ is hydrogen or (C₁-C₄)-alkyl,
R³² is hydrogen or (C₁-C₄)-alkyl,
or
R³¹ and R³² together with the atoms to which they are bonded form a 5- or 6-membered heterocycle,
L^{1B} is linear (C₂-C₁₀)-alkanediyl,
and
where (C₁-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
   B is a bond or a group of the formula

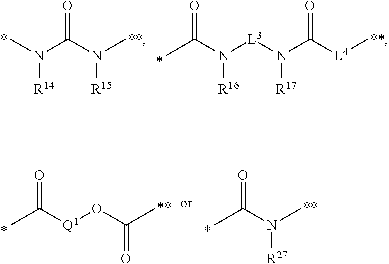

where
* marks the linkage site with L¹,
** marks the linkage site with L²,
P is O or NH,
L³ is a bond or (C₂-C₄)-alkanediyl,
L⁴ is a bond,
Q₁ is a 4- to 7-membered heterocycle,

Q² is a 3- to 7-membered carbocycle or a 4- to 7-membered heterocycle,
R¹⁴ is hydrogen or (C₁-C₄)-alkyl,
R¹⁵ is hydrogen or (C₁-C₄)-alkyl,
or
R¹⁴ and R¹⁵ together with the atoms to which they are bonded form a 5- or 6-membered heterocycle,
R¹⁶ is hydrogen or (C₁-C₄)-alkyl,
R¹⁷ is hydrogen or (C₁-C₄)-alkyl,
or
R¹⁶ and R¹⁷ together with the atoms to which they are bonded form a 5- or 6-membered heterocycle,
R¹⁸ is hydrogen or (C₁-C₄)-alkyl,
R¹⁹ is hydrogen or the side group of a natural α-amino acid or of its homologues or isomers,
R²⁰ is hydrogen or (C₁-C₄)-alkyl,
or
R¹⁹ and R²⁰ together with the atoms to which they are bonded form a pyrrolidinyl ring,
R²¹ is hydrogen or (C₁-C₄)-alkyl,
R²² is hydrogen or (C₁-C₄)-alkyl,
or
R²¹ and R²² together with the atoms to which they are bonded form a 3- to 7-membered carbocycle,
R²³ is (C₁-C₄)-alkyl,
R²⁴ is hydrogen or (C₁-C₄)-alkyl,
R²⁷ is hydrogen or (C₁-C₄)-alkyl,
   L² is linear (C₂-C₁₀)-alkanediyl or is a group of the formula

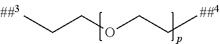

where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
   D is a group of the formula

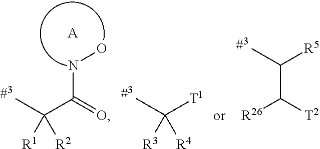

where
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula

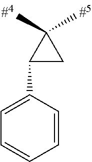

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

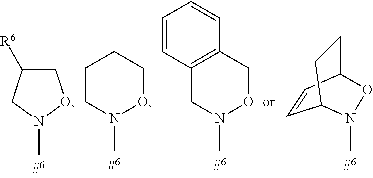

in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula

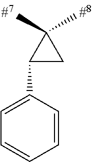

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   or
   R⁸ and R⁹ together with the nitrogen atom to which they are bonded form a 4- to 7-membered heterocycle,
   R¹⁰ is benzoyl,
   R¹¹ is benzyl, which may be substituted in the phenyl group by methoxycarbonyl or carboxyl,
R⁵ is hydrogen, methyl or a group of the formula

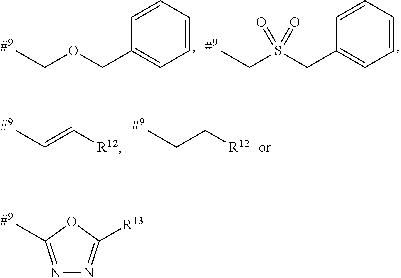

in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
   R³⁵ is methyl or hydroxy,
   and also their salts, solvates and solvates of the salts.
   Preferred in the context of the present invention are additionally also compounds of the formula (XXXa) in which
Cys is a cysteine residue which is bonded via the sulphur atom of the side chain via a carbon atom of the succinimide,
L¹ is a bond, linear (C₂-C₆)-alkanediyl, a group of the formula

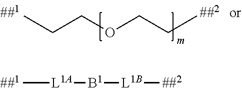

where
m is a number 2 or 3,
##¹ marks the linkage site with the group G,
##² marks the linkage site with the group B,
L^{1a} is linear (C₂-C₆)-alkanediyl,
B¹ is a group of the formula

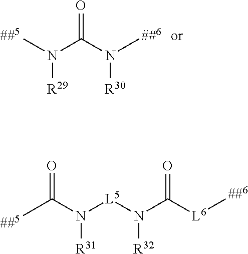

in which
##⁵ marks the linkage site with the group L^{1A},
##⁶ marks the linkage site with the group L^{1B},
L⁵ is a bond,
L⁶ is a bond,
R²⁹ is hydrogen,
R³⁰ is hydrogen,
R³¹ is hydrogen or methyl,
R³² is hydrogen or methyl,
L^{1B} is linear (C₂-C₆)-alkanediyl,
and
where (C₂-C₆)-alkanediyl may be substituted by 1 or 2 methyl substituents,
B is a bond or a group of the formula

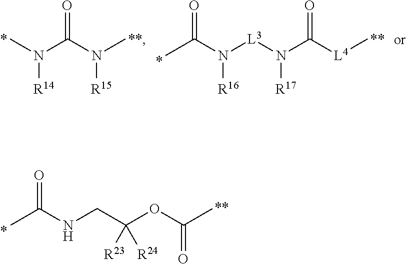

where
* marks the linkage site with L¹,
** marks the linkage site with L²,
L³ is a bond or ethane-1,2-diyl,
L⁴ is a bond,
R¹⁴ is hydrogen,
R¹⁵ is hydrogen,
R¹⁶ is hydrogen or methyl,
R¹⁷ is hydrogen or methyl,
or
R¹⁶ and R¹⁷ together with the atoms to which they are bonded form piperazinyl ring,
R²³ is methyl,
R²⁴ is hydrogen or methyl,
L² is linear (C₂-C₆)-alkanediyl or is a group of the formula

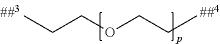

where
p is a number 2 or 3,
##³ marks the linkage site with the group B,
##⁴ marks the linkage site with the nitrogen atom,
D is a group of the formula

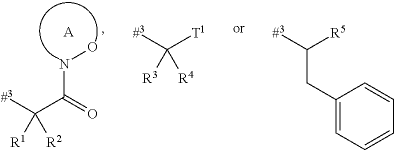

where
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula

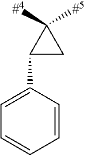

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

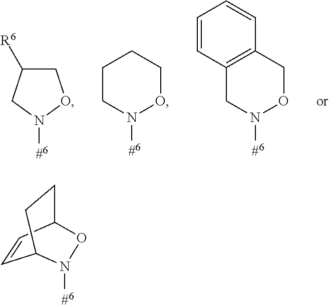

in which
#⁶ marks the linkage site with the carbonyl group,
R⁶ is hydrogen, hydroxy or benzyloxy,
R³ is hydrogen,
R⁴ is 1-hydroxyethyl, benzyl, 4-hydroxybenzyl, 1-phenylethyl or 1H-indol-3-ylmethyl,
or
R³ and R⁴ together with the carbon atom to which they are bonded form a (1S,2R)-2-phenylcyclopropane-1,1-diyl group of the formula

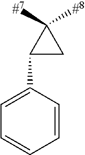

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   or
   R⁸ and R⁹ together with the nitrogen atom to which they are bonded form a 4- to 7-membered heterocycle,
   R¹⁰ is benzoyl,
   R¹¹ is benzyl which may be substituted in the phenyl group by methoxycarbonyl or carboxyl,
R⁵ is hydrogen, methyl or a group of the formula

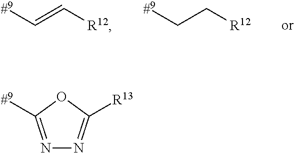

in which
#⁹ marks the linkage site with —CHCH₂phenyl,
R¹² is phenyl which may be substituted by methoxycarbonyl, carboxyl or a group of the formula —S(O)₂OH,
R¹³ is phenyl which may be substituted by methoxycarbonyl or carboxyl,
R³⁵ is methyl or hydroxy,
   and also their salts, solvates and solvates of the salts.

Particularly preferred in the context of the present invention are additionally also compounds of the formula (XXXa) in which
Cys is a cysteine residue which is bonded via the sulphur atom of the side chain via a carbon atom of the succinimide,
L¹ is a bond or linear (C₂-C₆)-alkanediyl,
B is a bond or a group of the formula

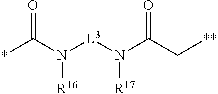

where
* marks the linkage site with L¹,
** marks the linkage site with L²,
L³ is a bond,
L⁴ is a bond,
R¹⁶ is hydrogen or methyl,
R¹⁷ is hydrogen or methyl,
L² is linear (C₂-C₆)-alkanediyl or is a group of the formula

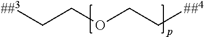

where
p is a number 2 or 3,
##³ marks the linkage site with the group B,
##⁴ marks the linkage site with the nitrogen atom,
D is a group of the formula

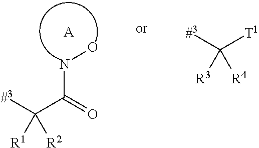

#³ marks the linkage site with the nitrogen atom,
R¹ is hydrogen,
R² is benzyl or 1H-indol-3-ylmethyl,
or
R¹ and R² together with the carbon atom to which they are bonded form a (1S,2R)-2-phenylcyclopropane-1,1-diyl group of the formula

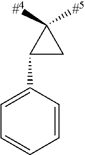

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

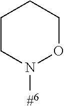

in which
#⁶ marks the linkage site with the carbonyl group,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula

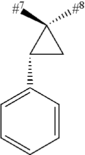

in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹,
in which
R⁷ is hydrogen,
R⁸ is hydrogen,
R⁹ is hydrogen,
R³⁵ is methyl,
   and also their salts, solvates and solvates of the salts.

The present invention additionally provides compounds of the formula (XXXI)

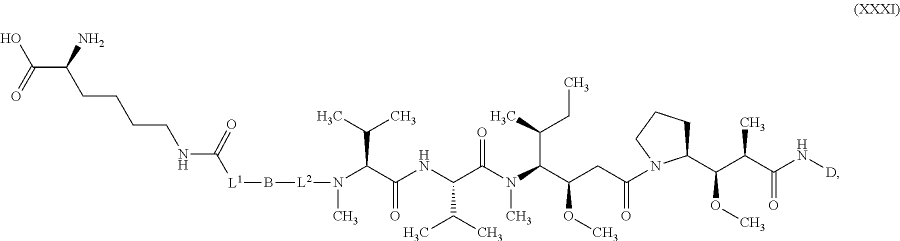

in which
L¹ is a bond, linear (C₁-C₁₀)-alkanediyl, a group of the formula

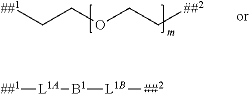

where
m is a number from 2 to 6,
##¹ marks the linkage site with the group G,
##² marks the linkage site with the group B,
L^{1A} is linear (C₂-C₁₀)-alkanediyl,
B¹ is a group of the formula

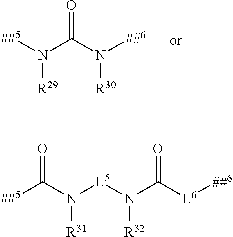

in which
##⁵ marks the linkage site with the group L^{1A},
##⁶ marks the linkage site with the group L^{1B},
L⁵ is a bond or (C₂-C₄)-alkanediyl,
L⁶ is a bond,
R²⁹ is hydrogen or (C₁-C₄)-alkyl,
R³⁰ is hydrogen or (C₁-C₄)-alkyl,
or
R²⁹ and R³⁰ together with the atoms to which they are bonded form a 5- or 6-membered heterocycle,
R³¹ is hydrogen or (C₁-C₄)-alkyl,
R³² is hydrogen or (C₁-C₄)-alkyl,
or
R³¹ and R³² together with the atoms to which they are bonded form a 5- or 6-membered heterocycle,
L^{1B} is linear (C₂-C₁₀)-alkanediyl,
and
where (C₁-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
B is a bond or a group of the formula

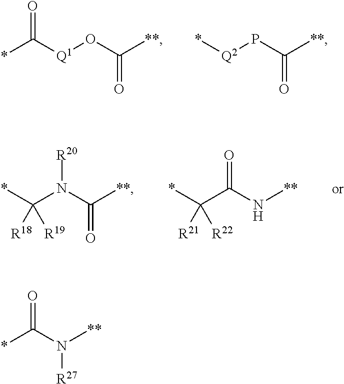

where
* marks the linkage site with L¹,
** marks the linkage site with L²,
P is O or NH,
Q¹ is a 4- to 7-membered heterocycle,
Q² is a 3- to 7-membered carbocycle or a 4- to 7-membered heterocycle,
R¹⁸ is hydrogen or (C₁-C₄)-alkyl,
R¹⁹ is hydrogen or the side group of a natural α-amino acid or of its homologues or isomers,
R²⁰ is hydrogen or (C₁-C₄)-alkyl,
or
R¹⁹ and R²⁰ together with the atoms to which they are bonded form a pyrrolidinyl ring,
R²¹ is hydrogen or (C₁-C₄)-alkyl,
R²² is hydrogen or (C₁-C₄)-alkyl,
or
R²¹ and R²² together with the atoms to which they are bonded form a 3- to 7-membered carbocycle,
R²⁷ is hydrogen or (C₁-C₄)-alkyl,
L² is linear (C₂-C₁₀)-alkanediyl or is a group of the formula

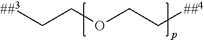

where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
D is a group of the formula

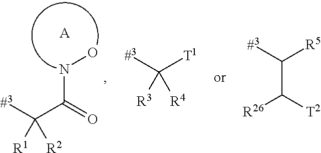

in which
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula

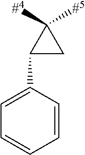

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

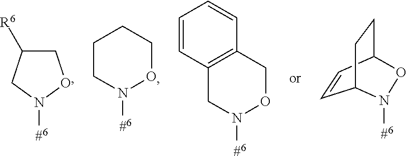

in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula

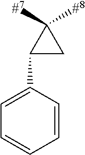

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   or
   R⁸ and R⁹ together with the nitrogen atom to which they are bonded form a 4- to 7-membered heterocycle,
   R¹⁰ is benzoyl,
   R¹¹ is benzyl, which may be substituted in the phenyl group by methoxycarbonyl or carboxyl,
R⁵ is hydrogen, methyl or a group of the formula

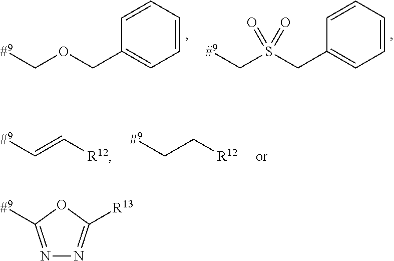

in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
R³⁵ is methyl or hydroxy,
   and also their salts, solvates and solvates of the salts.

Preferred in the context of the present invention are also compounds of the formula (XXXI) in which

L¹ is a bond, linear (C₂-C₆)-alkanediyl or a group of the formula

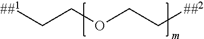

where
m is a number 2 or 3,
##¹ marks the linkage site with the group G,
##² marks the linkage site with the group B,
where (C₂-C₆)-alkanediyl may be substituted by 1 or 2 methyl substituents,
   B is a bond or a group of the formula

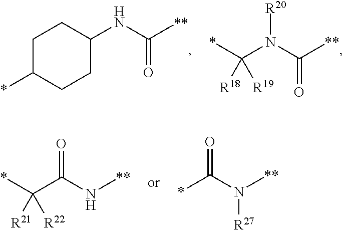

where
* marks the linkage site with L¹,
** marks the linkage site with L²,
R¹⁸ is hydrogen,
R¹⁹ is methyl, propan-2-yl, 2-methylpropan-1-yl or 1-methylpropan-1-yl,
R²⁰ is hydrogen or (C₁-C₄)-alkyl,
or
R¹⁹ and R²⁰ together with the atoms to which they are bonded form a pyrrolidinyl ring,
R²¹ is hydrogen or methyl,
R²² is hydrogen or methyl,
or
R²¹ and R²² together with the atoms to which they are bonded form a cyclopropyl ring,
R²⁷ is hydrogen or methyl,
   L² is linear (C₂-C₆)-alkanediyl or is a group of the formula

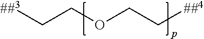

where
p is a number 2 or 3,
##³ marks the linkage site with the group B,
##⁴ marks the linkage site with the nitrogen atom,
where (C₂-C₁₀)-alkanediyl may be substituted by 1 or 2 methyl substituents,
and
where two carbon atoms of the alkanediyl chain in 1,4-relation to one another, with inclusion of any carbon atoms situated between them, may be bridged to form a phenyl ring,
   D is a group of the formula

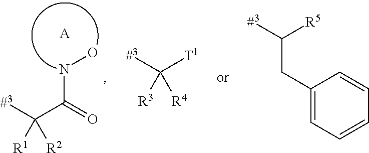

in which
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula

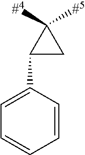

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

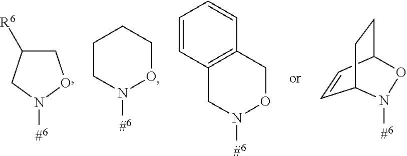

in which
#⁶ marks the linkage site with the carbonyl group,
R⁶ is hydrogen, hydroxy or benzyloxy,
R³ is hydrogen,
R⁴ is 1-hydroxyethyl, benzyl, 4-hydroxybenzyl, 1-phenylethyl or 1H-indol-3-ylmethyl,
or
R³ and R⁴ together with the carbon atom to which they are bonded form a (1S,2R)-2-phenylcyclopropan-1,1-diyl group of the formula

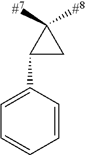

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   or
   R⁸ and R⁹ together with the nitrogen atom to which they are bonded form a 4- to 7-membered heterocycle,
   R¹⁰ is benzoyl,
   R¹¹ is benzyl, which may be substituted in the phenyl group by methoxycarbonyl or carboxyl,
R⁵ is hydrogen, methyl or a group of the formula

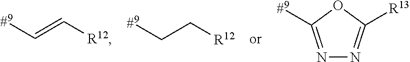

in which
#⁹ marks the linkage site with CHCH₂phenyl,
R¹² is phenyl which may be substituted by methoxycarbonyl, carboxyl or a group of the formula —S(O)₂OH,
R¹³ is phenyl which may be substituted by methoxycarbonyl or carboxyl,
   R³⁵ is methyl or hydroxy,
   and also their salts, solvates and solvates of the salts.

Particularly preferred in the context of the present invention are also compounds of the formula (XXXI) in which

L¹ is a bond,
B is a bond,
L² is linear (C₂-C₆)-alkanediyl or is a group of the formula

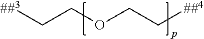

where
p is a number 2 or 3,
##³ marks the linkage site with the group B,
##⁴ marks the linkage site with the nitrogen atom,
   D is a group of the formula

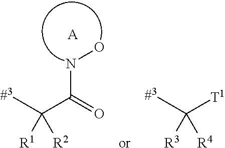

where
#³ marks the linkage site with the nitrogen atom,
R¹ is hydrogen,
R² is benzyl, 4-hydroxybenzyl or 1H-indol-3-ylmethyl,
or
R¹ and R² together with the carbon atom to which they are bonded form a (1S,2R)-2-phenylcyclopropane-1,1-diyl group of the formula

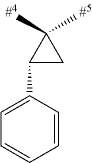

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

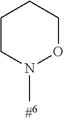

in which
#⁶ marks the linkage site with the carbonyl group,
R⁶ is hydrogen, hydroxy or benzyloxy,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula

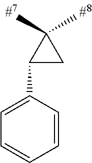

in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹,
   in which
   R⁷ is hydrogen,
   R⁸ is hydrogen,
   R⁹ is hydrogen,
      R³⁵ is methyl,
      and also their salts, solvates and solvates of the salts.

Preferred in the context of the present invention are also compounds of the formula (Ia), in which

### AK is AK₁

where
AK₁ is an antibody or an antigen-binding antibody fragment which binds to FGFR2 and is bonded via the sulphur atom of a cysteine residue of the binder to the group G,
   G is a group of the formula

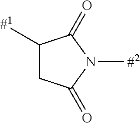

where
#¹ marks the linkage site with the cysteine residue of the binder,
#2 marks the linkage site with the group L¹,
   and
   n, L¹, B, L², D and R³⁵ have the definitions indicated above,
   and also their salts, solvates and solvates of the salts.

Preferred in the context of the present invention are also compounds of the formula (Ia), in which

### AK is AK₂

where
AK₂ is an antibody or an antigen-binding antibody fragment which binds to FGFR2 and is bonded via the NH side group of a lysine residue of the binder to the group G,
   G is carbonyl,
   and
   n, L¹, B, L², D and R³⁵ have the definitions indicated above,
   and also their salts, solvates and solvates of the salts.

Preference in the context of the present invention is also given to compounds of the formula (Ia), in which

### AK is AK₁

where
AK₁ is a binder which binds FGFR2, and which is bonded via the sulphur atom of a cysteine residue of the binder to the group G,
   G is a group of the formula

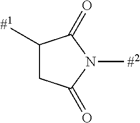

where
#¹ marks the linkage site with the cysteine residue of the binder,
#² marks the linkage site with the group L¹,
   and
   n, L¹, B, L², D and R³⁵ have the definitions indicated above,
   and also their salts, solvates and solvates of the salts.

Preference in the context of the present invention is also given to compounds of the formula (Ia), in which

### AK is AK₂

where
AK₂ is an a binder which binds FGFR2 and which is bonded via the NH side group of a lysine residue of the binder to the group G,
   G is carbonyl,
   and
   n, L¹, B, L², D and R³⁵ have the definitions indicated above,
   and also their salts, solvates and solvates of the salts.

Preference in the context of the present invention is also given to compounds of the general formula (Ia), in which

### AK is AK₂

where
AK₂ is a binder which binds FGFR2 and which is bonded via the NH side group of a lysine residue of the binder to the group G,
   G is carbonyl,
   L¹ is a bond,
   B is a bond,
   L² is linear (C₃-C₆)-alkanediyl or is a group of the formula

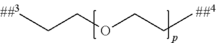

where
p is a number 2 or 3,
##³ marks the linkage site with the group B,
##⁴ marks the linkage site with the nitrogen atom,
   n, D and R³⁵ have the definitions indicated above,
   and also their salts, solvates and solvates of the salts.

Preference in the context of the present invention is also given to compounds of the general formula (Ia), in which

### AK is AK₁

where
AK₁ is a binder which binds FGFR2 and which is attached via the sulphur atom of a cysteine residue of the binder to the group G,
   G is a group of the formula

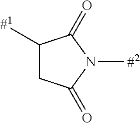

where
#¹ marks the linkage site with the cysteine residue of the binder, #² marks the linkage site with the group L¹,
   L¹ is a bond, linear (C₃-C₅)-alkanediyl or a group of the formula

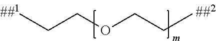

where
m is a number 2 or 3,
##¹ marks the linkage site with the group G,
##2 marks the linkage site with the group B,
where (C₃-C₅)-alkanediyl may be substituted by 1 or 2 methyl substituents,
   B is a bond or a group of the formula

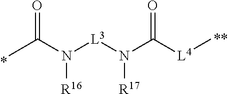

where
* marks the linkage site with L¹,
** marks the linkage site with L²,
L³ is a bond or ethane-1,2-diyl,
L⁴ is a bond or a group of the formula

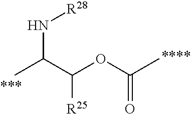

in which
*** marks the linkage site with the carbonyl group,
**** marks the linkage site with L²,
R²⁵ is methyl,
R²⁸ is hydrogen, methylcarbonyl or tert-butyloxycarbonyl,
R¹⁶ is hydrogen or methyl,
R¹⁷ is hydrogen or methyl,
or
R¹⁶ and R¹⁷ together with the atoms to which they are bonded form a piperazinyl ring,
   L² is linear (C₃-C₅)-alkanediyl or is a group of the formula

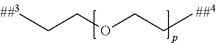

where
p is a number 2 or 3,
##³ marks the linkage site with the group B,
##⁴ marks the linkage site with the nitrogen atom,
   and
   n, D and R³⁵ have the definitions indicated above,
   and also their salts, solvates and solvates of the salts.

Preferred in the context of the present invention are also compounds of the formula (Ia), (XXXa) and (XXXI), in which

L¹ is a bond,
B is a bond,
L² is linear (C₃-C₆)-alkanediyl or is a group of the formula

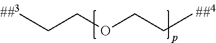

where
P is a number 2 or 3,
##³ marks the linkage site with the group B,
##⁴ marks the linkage site with the nitrogen atom,
   and
   n, AK, Cys, G, D and R³⁵ have the definitions indicated above,
   and also their salts, solvates and solvates of the salts.

Preferred in the context of the present invention are also compounds of the formula (Ia), in which

L¹ is linear (C₁-C₁₀)-alkanediyl or a group of the formula

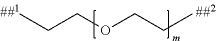

where
m is a number from 2 to 6,
##¹ marks the linkage site with the group G,
##² marks the linkage site with the group B,
where (C₁-C₁₀)-alkanediyl may be substituted by 1 to 4 substituents selected independently of one another from the group consisting of methyl, hydroxyl and benzyl,
   B is a bond or a group of the formula

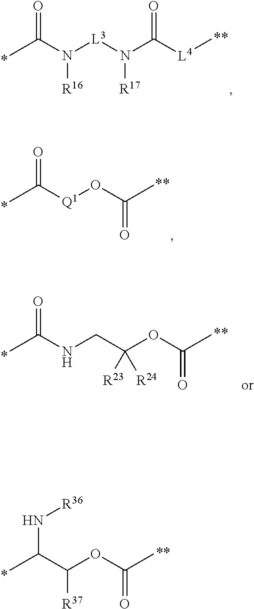

where
* marks the linkage site with L¹,
** marks the linkage site with L²,
L³ is a bond or (C₂-C₄)-alkanediyl,
L⁴ is a group of the formula

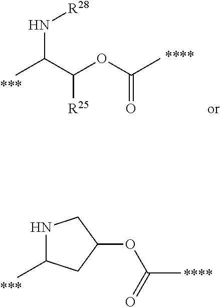

in which
*** marks the linkage site with the carbonyl group,
**** marks the linkage site with L²,
R²⁵ is hydrogen or methyl,
R²⁸ is hydrogen, (C₁-C₄)-alkylcarbonyl, tert-butyloxycarbonyl or benzyloxycarbonyl,
Q¹ is a 4- to 7-membered heterocycle,
R¹⁶ is hydrogen or (C₁-C₄)-alkyl,
R¹⁷ is hydrogen or (C₁-C₄)-alkyl,
or
R¹⁶ and R¹⁷ together with the atoms to which they are bonded form a 5- or 6-membered heterocycle,
R²³ is (C₁-C₄)-alkyl,
R²⁴ is hydrogen or (C₁-C₄)-alkyl,
R³⁶ is hydrogen, (C₁-C₄)-alkylcarbonyl, tert-butyloxycarbonyl or benzyloxycarbonyl,
R³⁷ is hydrogen or methyl,
or
R³⁶ and R³⁷ together with the atoms to which they are bonded form a pyrrolidine ring,
   L² is linear (C₂-C₁₀)-alkanediyl or is a group of the formula

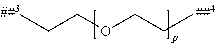

where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxyl and benzyl,
   and
   n, AK, G, D and R³⁵ have the definitions indicated above,
   and also their salts, solvates and solvates of the salts.

Preferred in the context of the present invention are also compounds of the formula (Ia), in which

L¹ is linear (C₂-C₆)-alkanediyl or a group of the formula

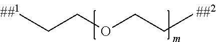

where
m is a number 2 or 3,
##¹ marks the linkage site with the group G,
##² marks the linkage site with the group B,
   B is a bond or a group of the formula

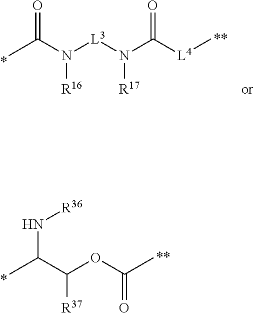

where
* marks the linkage site with L¹,
** marks the linkage site with L²,
L³ is a bond or ethane-1,2-diyl,
L⁴ is a group of the formula

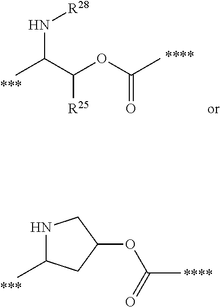

where
*** marks the linkage site with the carbonyl group,
**** marks the linkage site with L²,
R²⁵ is hydrogen or methyl,
R²⁸ is hydrogen, methylcarbonyl or tert-butyloxycarbonyl,
R¹⁶ is hydrogen or methyl,
R¹⁷ is hydrogen or methyl,
or
R¹⁶ and R¹⁷ together with the atoms to which they are bonded form a piperazinyl ring,
R³⁶ is hydrogen, methylcarbonyl or tert-butyloxycarbonyl,
R³⁷ is hydrogen or methyl,
or
R³⁶ and R³⁷ together with the atoms to which they are bonded form a pyrrolidine ring,
   L² is linear (C₂-C₆)-alkanediyl or is a group of the formula

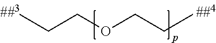

where
p is a number 2 or 3,
##³ marks the linkage site with the group B,
##⁴ marks the linkage site with the nitrogen atom,
   and
   n, AK, G, D and R³⁵ have the definitions indicated above,
   and also their salts, solvates and solvates of the salts.

Preferred in the context of the present invention are also compounds of the formula (Ia) and (XXXa), in which

G is a group of the formula

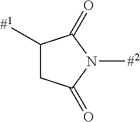

in which
#¹ marks the linkage site with the cysteine residue of the binder,
#2 marks the linkage site with the group L¹,
   L¹ is linear (C₃-C₅)-alkanediyl or a group of the formula

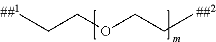

in which
m is a number 2 or 3,
##¹ marks the linkage site with the group G,
##2 marks the linkage site with the group B,
where (C₃-C₅)-alkanediyl may be substituted by 1 or 2 methyl substituents,
   B is a bond or a group of the formula

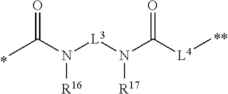

where
* marks the linkage site with L¹,
** marks the linkage site with L²,
L³ is a bond or ethane-1,2-diyl,
L⁴ is a bond,
   L² is linear (C₃-C₅)-alkanediyl or is a group of the formula

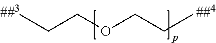

where
p is a number 2 or 3,
##³ marks the linkage site with the group B,
##⁴ marks the linkage site with the nitrogen atom,
   and
   n, AK₁, Cys, D, R¹⁶ and R¹⁷ have the definitions indicated above,
   and also their salts, solvates and solvates of the salts.

Preferred in the context of the present invention are also compounds of the formula (Ia), (XXXa) and (XXXI), in which

D is a group of the formula

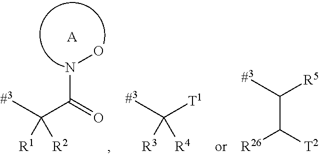

where
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula

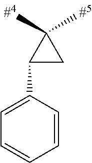

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

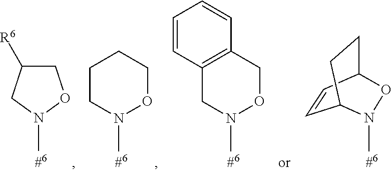

in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula

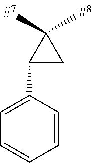

in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
   in which
   R⁷ is hydrogen, methyl, ethyl, n-propyl, tert-butyl, benzyl or adamantylmethyl,
   R⁸ is hydrogen or methyl,
   R⁹ is hydrogen, methyl, ethyl, n-propyl or benzyl,
   or
   R⁸ and R⁹ together with the nitrogen atom to which they are bonded form a 4- to 7-membered heterocycle,
   R¹⁰ is benzoyl,
   R¹¹ is benzyl, which may be substituted in the phenyl group by methoxycarbonyl or carboxyl
R⁵ is hydrogen, methyl or a group of the formula

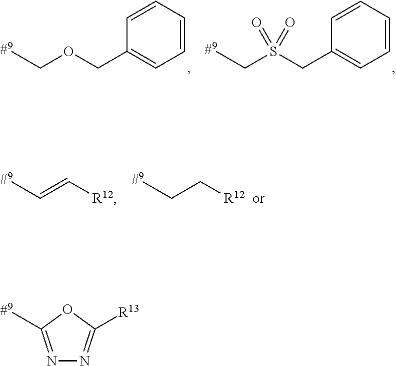

in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen,
T² is phenyl, benzyl, 1H-indol-3-yl or 1H-indol-3-ylmethyl,
   and
   n, AK, Cys, G, L¹, B, L², D and R³⁵ have the definitions indicated above,
   and also their salts, solvates and solvates of the salts.

Preferred in the context of the present invention are also compounds of the formula (Ia), (XXXa) and (XXXI), in which

D is a group of the formula

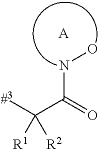

where
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula

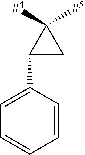

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

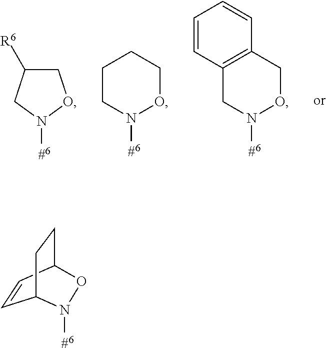

in which
#⁶ marks the linkage site with the carbonyl group,
R⁶ is hydrogen, hydroxy or benzyloxy,
   and
   n, AK, Cys, G, L¹, B, L² and R³⁵ have the definitions indicated above,
   and also their salts, solvates and solvates of the salts.

Preferred in the context of the present invention are also compounds of the formula (Ia), (XXXa) and (XXXI), in which

D is a group of the formula

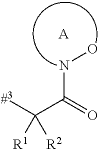

where
#³ marks the linkage site with the nitrogen atom,
R¹ is hydrogen,
R² is benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula

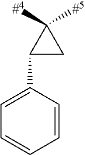

in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula

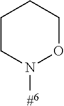

in which
#⁶ marks the linkage site with the carbonyl group,
   and
   n, AK, Cys, G, L¹, B, L² and R³⁵ have the definitions indicated above,
   and also their salts, solvates and solvates of the salts.

Preferred in the context of the present invention are also compounds of the formula (Ia), (XXXa) and (XXXI), in which

R³⁵ is hydroxyl,
and
n, AK, Cys, G, L¹, B, L², D and R³⁵ have the definitions indicated above,
and also their salts, solvates and solvates of the salts.

Preferred in the context of the present invention are also compounds of the formula (Ia), (XXXa) and (XXXI), in which

R³⁵ is methyl,
and
n, AK, Cys, G, L¹, B, L², D and R³⁵ have the definitions indicated above,
and also their salts, solvates and solvates of the salts.

A preferred subject of the present invention are binder-drug conjugates of the general formula (Ia) in which D can have the following structures and * stands for the linkage site with the nitrogen atom:

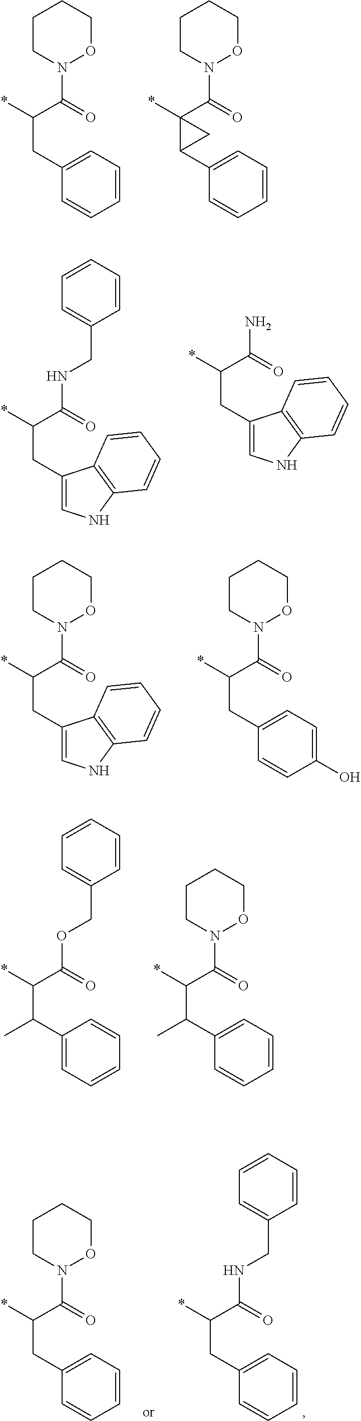

and also their salts, solvates and solvates of the salts.

A preferred subject of the present invention are binder-drug conjugates of the general formula (Ia) in which D has a structure which is disclosed by one of the intermediates of the present invention; and the linker unit §-G-L¹-B-L²-§§ and also all other variables are defined in accordance with the present invention; and their salts, solvates and solvates of the salts. AK is preferably an anti-FGFR2 antibody or antigen-binding fragment thereof.

A preferred subject of the present invention are binder-drug conjugates of the general formula (Ia) in which the linker-drug unit has a structure which is disclosed by one of the intermediates or examples of the present invention; and their salts, solvates and solvates of the salts. AK is preferably an anti-FGFR2 antibody or antigen-binding fragment thereof.

A preferred subject of the present invention are binder-drug conjugates of the general formula (Ia) in which the linker-drug unit has a structure which is disclosed by one of the examples of the present invention; and their salts, solvates and solvates of the salts. AK is preferably an anti-FGFR2 antibody or antigen-binding fragment thereof.

Particularly preferred subject of the present invention are binder-drug conjugates of the general formula (Ia)

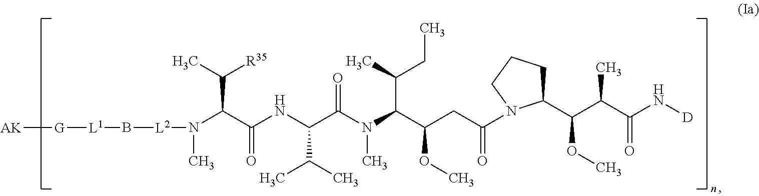

in which
n is a number from 1 to 50,
AK is a binder which binds to FGFR2,
the group §-G-L¹-B-§§ is a linker,
where
   § marks the linkage site with the group AK and
   §§ marks the linkage site with the nitrogen atom,
      L² is linear (C₂-C₁₀)-alkanediyl or is a group of the formula

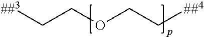

where
p is a number from 2 to 6,
##³ marks the linkage site with the group B,
##⁴ marks the linkage site with the nitrogen atom,
where (C₂-C₁₀)-alkanediyl may be substituted by 1 to 4 methyl substituents selected independently of one another from the group consisting of methyl, hydroxyl and benzyl
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
   D is a group of the following formula, where * is the linkage site to the nitrogen atom

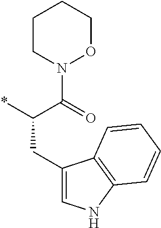 [SMILES: *[C@@H](Cc1c[nH]c2ccccc12)C(=O)N1CCCCO1]

and also their salts, solvates and solvates of the salts.
Particularly preferred subject of the present invention are compounds of the following formula

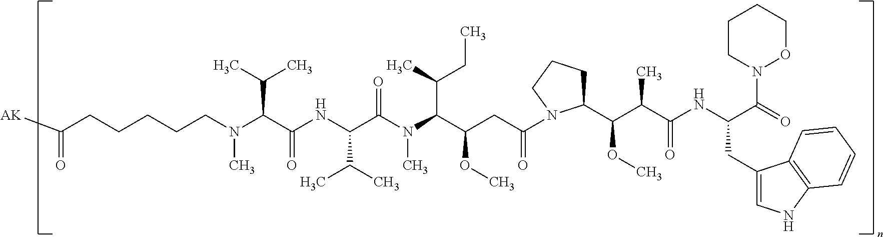

where AK is a binder which binds FGFR2, and n is a number from 1 to 10, and also their salts, solvates and solvates of the salts. It is preferred if the binder is bonded via a NH side group of a lysine residue to the linker-toxophore unit.

Particularly preferred subject of the present invention are compounds of the following formula

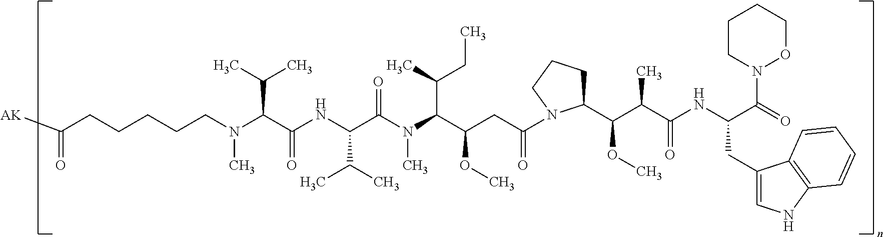

where AK is an antibody or an antibody fragment which binds FGFR2, and n is a number from 1 to 10, and also their salts, solvates and solvates of the salts. It is preferred if the antibody or antibody fragment is bonded via an NH side group of a lysine residue of the antibody or antibody fragment to the linker-toxophore unit.

Particularly preferred subject of the present invention is the compound of the following formula

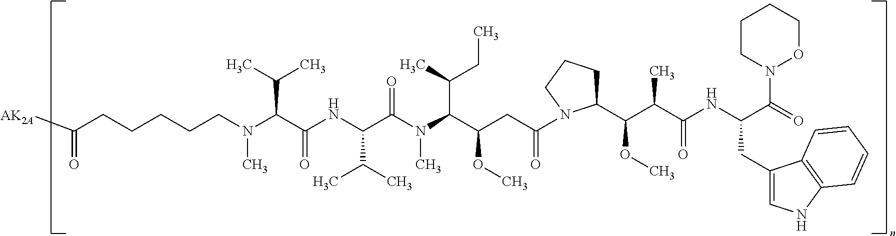

where AK2A is M048-D01-hIgG1 and n is a number from 1 to 10, and also the salts, solvates and solvates of the salts thereof.

A further particularly preferred subject of the present invention is the compound of the following formula

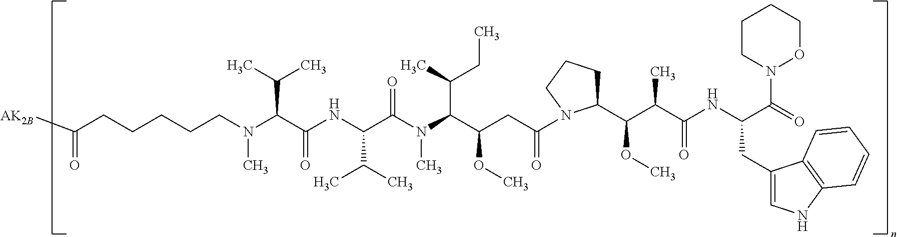

where AK2B is M048-D01-hIgG1-b and n is a number from 1 to 10, and also the salts, solvates and solvates of the salts thereof.

The definitions of radicals that are indicated individually in the respective combinations and preferred combinations of radicals are also replaced arbitrarily by radical definitions of other combinations, independently of the respective combinations of radicals that are indicated.

Especially preferred are combinations of two or more of the abovementioned preference ranges.

Further provided by the invention is a process for preparing the compounds of the invention of the formula (Ia), characterized in that a solution of the binder in PBS buffer
[A] is admixed with a suitable reducing agent, such as, for example, dithiothreitol or tris(2-carboxyethyl)phosphine hydrochloride, and is subsequently reacted with a compound of the formula (IIa)

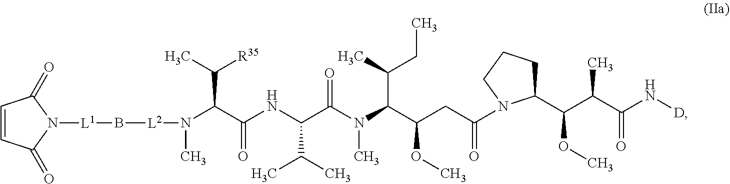

in which D, L¹, B, L² and R³⁵ each have the definitions indicated above,
to give a compound of the formula (I-A)

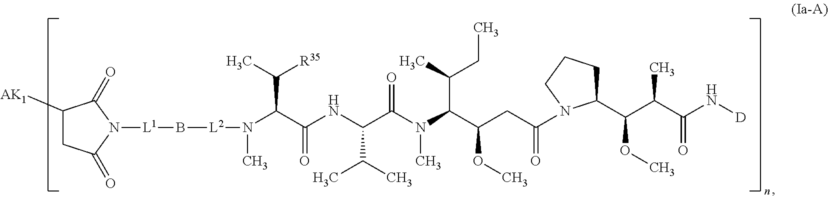

in which n, AK₁, D, L¹, B, L² and R³⁵ each have the definitions indicated above,
   or
[B] is reacted with a compound of formula (IIIa)

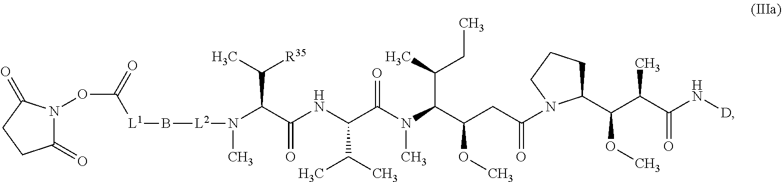

in which D, L¹, B, L² and R³⁵ each have the definitions indicated above,
to give a compound of the formula (Ia-B)

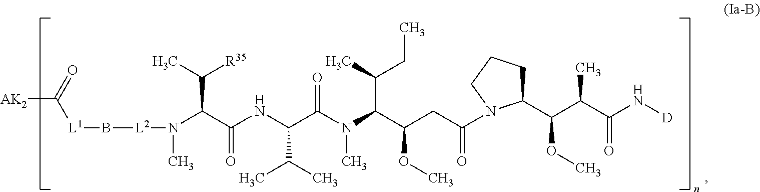

in which n, AK₂, D, L¹, B, L² and R³⁵ each have the definitions indicated above.

### Cysteine Coupling:

The partial reduction of the antibody and also the subsequent conjugation of the (partially) reduced antibody with a compound of the formula (IIa) takes place in accordance with the methods known to the skilled person, see e.g. Ducry et. al., Bioconj. Chem. 2010, 21, 5 and references herein, Klussman et. al., Bioconj. Chem. 2004, 15(4), 765-773. The mild reduction of the antibody is accomplished preferably by addition of 2-6 equivalents of TCEP to the antibody, which is present in a suitable buffer solution, preferably phosphate buffer, and by stirring for 30-180 minutes at temperatures between 15 and 40° C., preferably at RT. This is followed by the conjugation, by addition of a solution of a compound of the formula (Ha) in DMSO, acetonitrile or DMF to the solution of the (partially) reduced antibody in PBS buffer, and subsequent reaction at a temperature of 0° C. to +40° C., more particularly of +10° C. to +30° C., for a period of 30 minutes to 6 hours, more particularly 1 to 2 hours.

### Lysine Coupling:

First of all the compounds of the formula (IIIa) or comparable activated carboxyl components are prepared by conventional methods of peptide chemistry. They are then taken up in inert solvents such as DMSO or DMF, for example, and added to the antibody, which is preferably present in phosphate buffer at a neutral pH. The solution is stirred for 1-16 h at a temperature between 15 and 40° C., preferably RT.

The preparation processes described above are elucidated by way of example using the schemes below (Scheme 1 and 2):

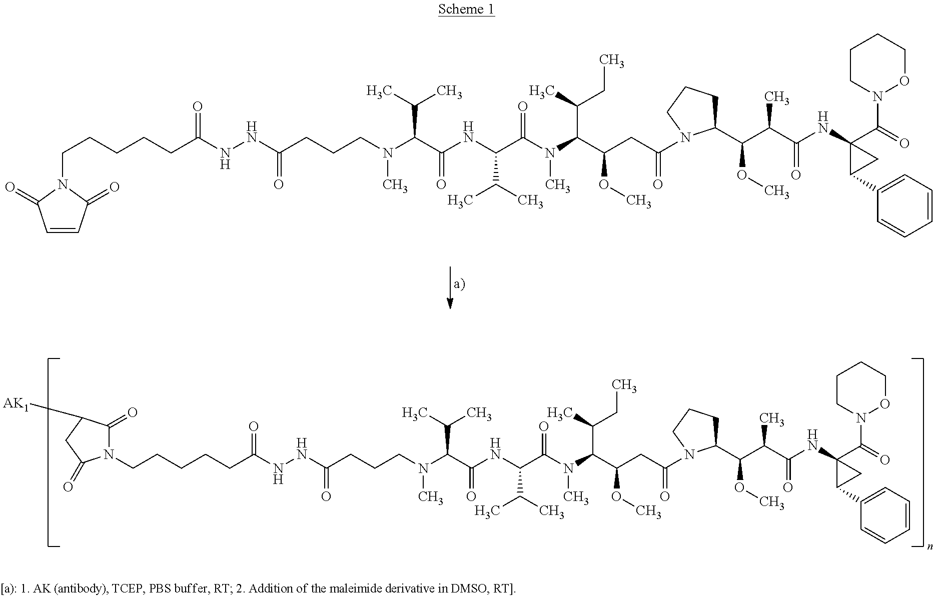

72

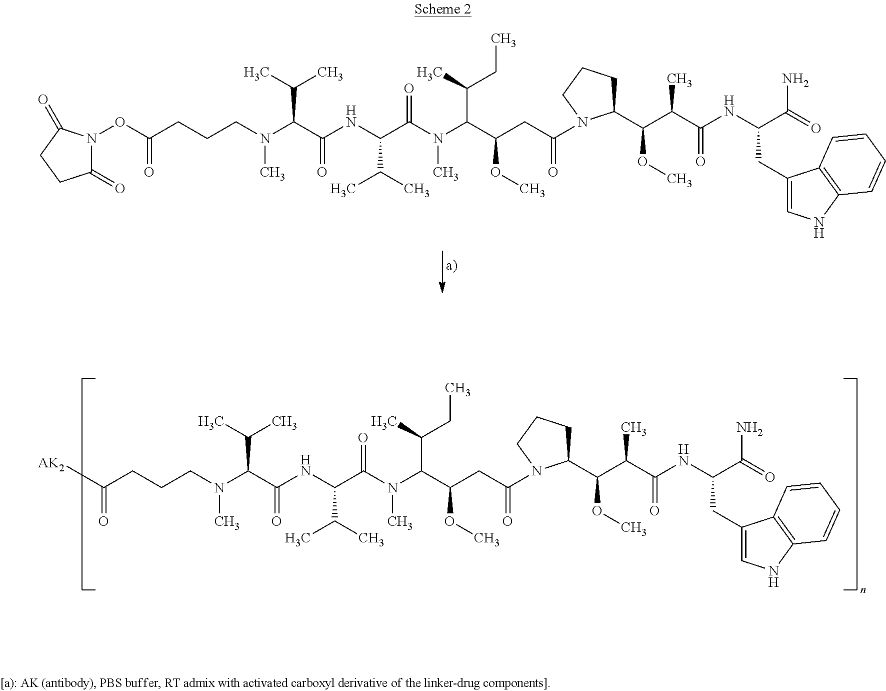

The compounds of the formula (II) in which L¹ and B are a bond can be prepared by subjecting a compound of the formula (IV)

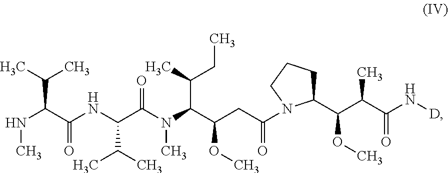

in which D has the definition indicated above,
to reductive amination in an inert solvent with a compound of the formula (V)

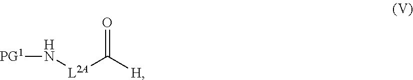

in which
L^{2A} has the above-defined definition of L², but is shortened by one carbon atom in the alkyl chain length,
PG¹ is an amino-protective group such as, for example, (9H-fluoren-9-ylmethoxy)carbonyl, tert-butoxycarbonyl or benzyloxycarbonyl,
   to give a compound of the formula (VI)

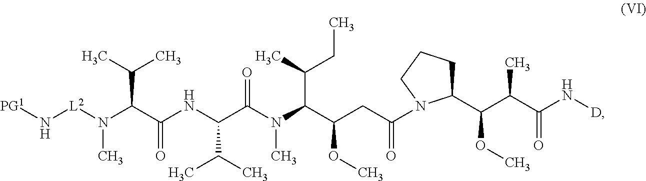

in which D, L² and PG¹ have the definition indicated above,
eliminating the protective group PG¹ from this compound by methods known to the skilled person, and reacting the deprotected compound in an inert solvent in the presence of a suitable base with methyl 2,5-dioxo-2,5-dihydro-1H-pyrrole-1-carboxylate to give a compound of the formula (II-A)

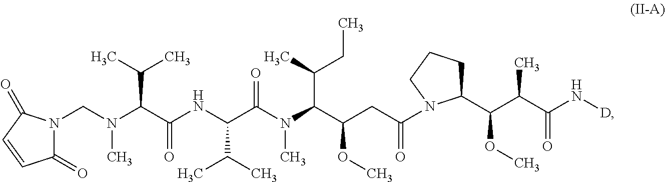

in which D and L² each have the definitions indicated above.

The compounds of the formula (II) in which B is a group of the formula (B¹)

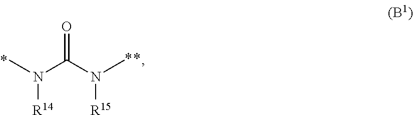

in which *, **, R¹⁴ and R¹⁵ each have the conditions indicated above,
can be prepared by eliminating the protective group PG¹ from a compound of the formula (VI) by methods known to the skilled person, and reacting the deprotected compound in an inert solvent in the presence of a suitable base with a compound of the formula (VII)

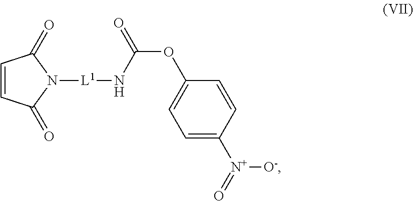

in which L¹ has the definition indicated above,
to give a compound of the formula (II-B)

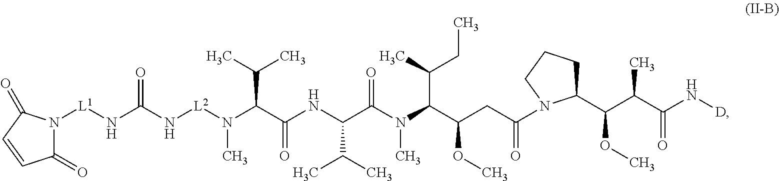

in which D, L¹ and L² each have the definitions indicated above.

The compounds of the formula (II) in which B is a group of the formula (B²)

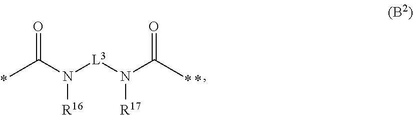

in which *, **, L³, R¹⁶ and R¹⁷ each have the conditions indicated above can be prepared by subjecting a compound of the formula (IV)
to reductive amination in an inert solvent with a compound of the formula (VIII)

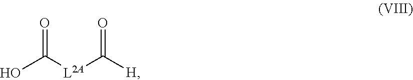

in which
L^{2A} has the above-defined definition of L², but is shortened by one carbon atom in the alkyl chain length,
   to give a compound of the formula (IX)

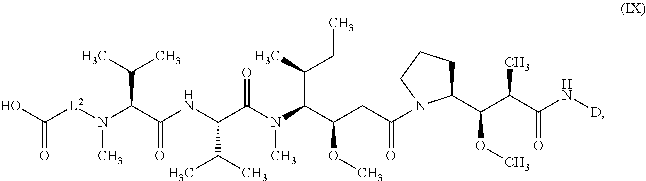

in which D and L² have the definitions indicated above,
and reacting this compound in an inert solvent in the presence of a suitable coupling reagent and a suitable base with a compound of the formula (X)

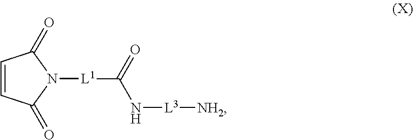

in which L¹ and L³ each have the definitions indicated above,
to give a compound of the formula (II-C)

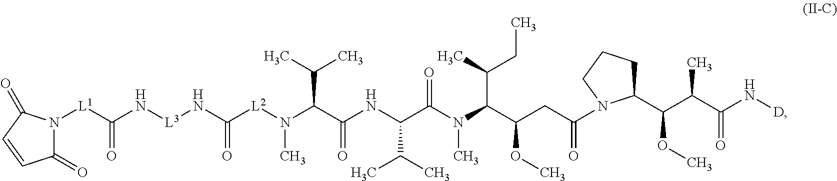

in which D, L¹, L² and L³ each have the definitions indicated above.

Compound of the formula (II), in which B is a group of the formula (B³)

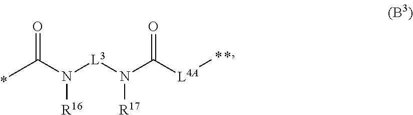

in which *, **, L³, R¹⁶ and R¹⁷ each have the conditions indicated above and
L^{4A} is a group of the formula

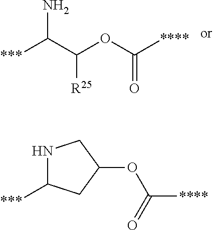

in which
*** marks the linkage site with the carbonyl group,
**** marks the linkage site with L²,
R²⁵ is hydrogen or methyl,
   can be prepared by reacting a compound of the formula (IX) in an inert solvent in the presence of a suitable base and a suitable coupling reagent with a compound of the formula (XI-A) or (XI-B)

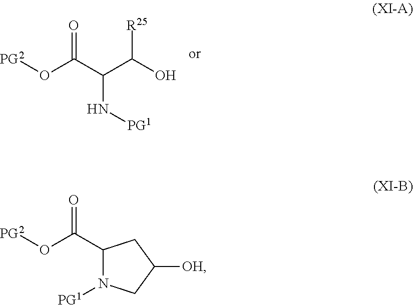

in which R²⁵ and PG¹ each have the definitions indicated above and
PG² is a suitable carboxyl-protective group, more particularly benzyl,
to give a compound (XII-A) or (XII-B)

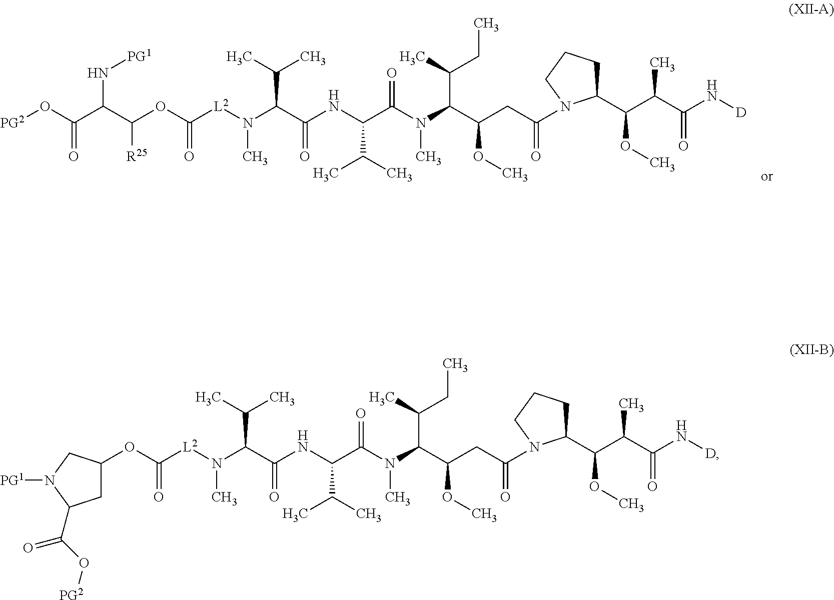

in which D, PG¹, PG² and L² have the definitions indicated above,
eliminating the protective group PG² from this compound subsequently, by methods known to the skilled person, and reacting the deprotected compound in an inert solvent in the presence of a suitable coupling reagent and a suitable base with a compound of the formula (X), and finally, eliminating the protective group PG1 from this compound, by methods known to the skilled person, to give a compound of the formula (II-D-A) or (II-D-B)

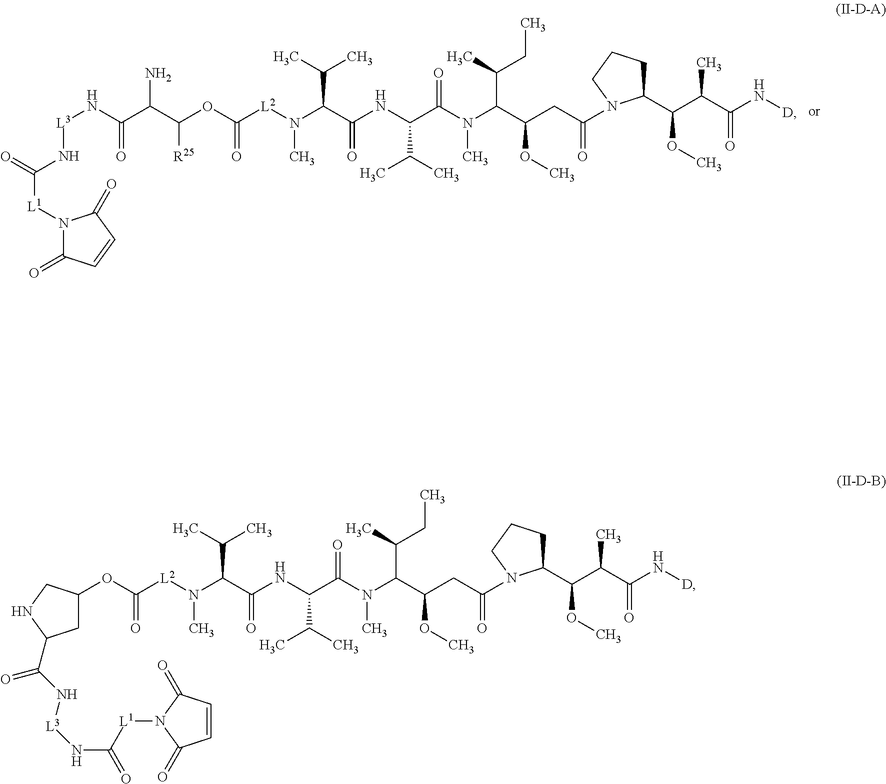

in which D, L¹, L² and L³ have the definitions indicated above.

Compound of the formula (II), in which B is a group of the formula (B⁴)

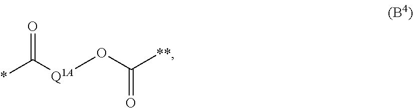

in which *, ** each have the conditions indicated above and
Q^{1A} is an N-linked 4- to 7-membered heterocycle,
can be prepared by reacting a compound of the formula (IX) in an inert solvent in the presence of a suitable base and a suitable coupling reagent with a compound of the formula (XXI)

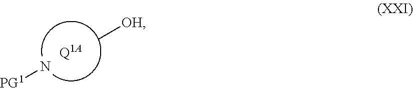

in which PG¹ and Q^{1A} each have the definitions indicated above,
to give a compound of the formula (XXII)

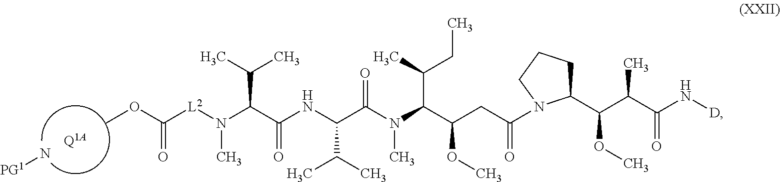

in which PG¹, Q^{1A}, D and L² have the definitions indicated above,
eliminating the protective group PG¹ from this compound, by methods known to the skilled person, and subsequently reacting the deprotected compound in an inert solvent in the presence of a suitable coupling reagent and a suitable base with a compound of the formula (XXIII)

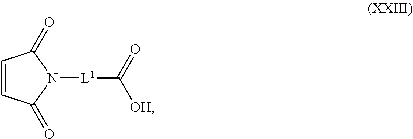

in which L¹ has the definition indicated above,
to give a compound of the formula (II-D)

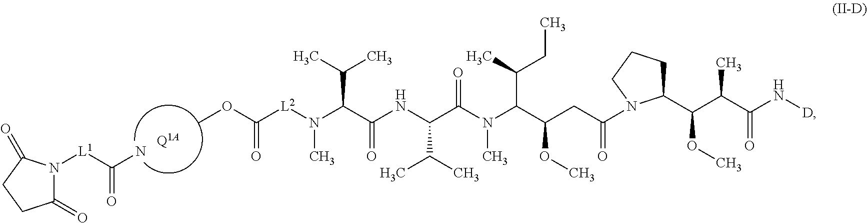

in which Q^{1A}, D, L¹ and L² have the definitions indicated above.

The compounds of the formula (III), in which L¹ and B are a bond can be prepared by reacting a compound of the formula (IX) in an inert solvent in the presence of a suitable coupling reagent and a suitable base with N-hydroxysuccinimide to give a compound of the formula (III-A)

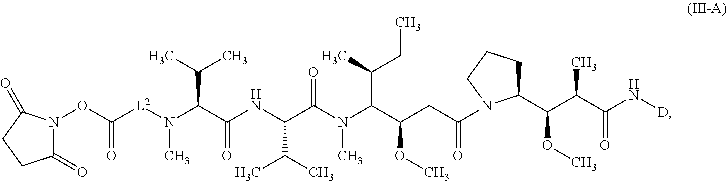

in which D and L² each have the definitions indicated above.

The compounds of the formula (III), in which L¹ is a bond and B is a group of the formula (B^{5A})

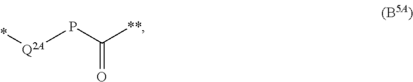

in which *, ** and P each have the definitions indicated above and
Q^{2A} is a 3- to 7-membered carbocycle,
can be prepared by reacting a compound of the formula (IX) in an inert solvent in the presence of a suitable coupling reagent and a suitable base with a compound of the formula (XIII)

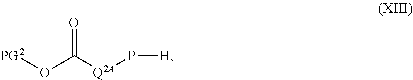

in which P, Q^{2A} and PG² each have the definitions indicated above,
to give a compound of the formula (XIV)

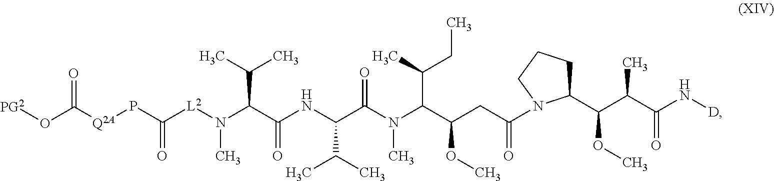

in which D, P, Q^{2A}, L² and PG² each have the definitions indicated above,
eliminating the protective group PG² from this compound by methods known to the skilled person, and subsequently reacting the deprotected compound in an inert solvent in the presence of a suitable base with N-hydroxysuccinimide to give a compound of the formula (III-B)

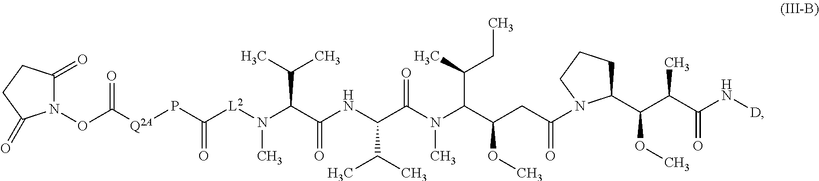

in which D, P, Q^{2A} and L² each have the definitions indicated above.

The compounds of the formula (III), in which L¹ is a bond and B is a group of the formula (B⁶)

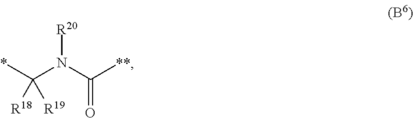

in which *, **, R¹⁸, R¹⁹ and R²⁰ each have the definitions indicated above,
can be prepared by reacting a compound of the formula (IX) in an inert solvent in the presence of a suitable coupling reagent and a suitable base with a compound of the formula (XV)

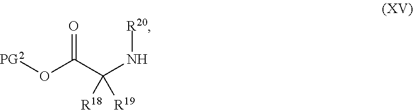

in which R¹⁸, R¹⁹, R²⁰ and PG² each have the definitions indicated above,
to give a compound of the formula (XVI)

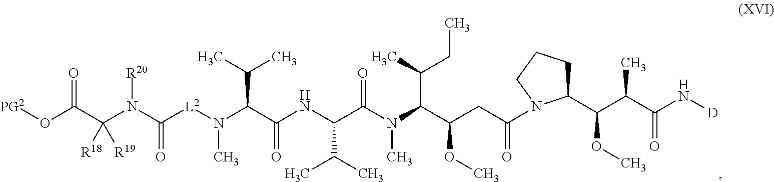

in which D, R¹⁸, R¹⁹, R²⁰, L² and PG² each have the definitions indicated above,
eliminating the protective group PG² from this compound by methods known to the skilled person, and subsequently reacting the deprotected compound in an inert solvent in the presence of a suitable coupling reagent and a suitable base with N-hydroxysuccinimide to give a compound of the formula (III-C)

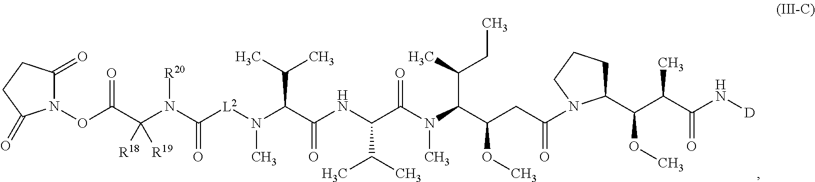

in which D, R¹⁸, R¹⁹, R²⁰ and L² each have the definitions indicated above.

The compounds of the formula (III), in which L¹ is a bond and B is a group of the formula (B⁷)

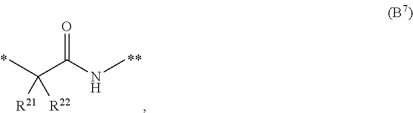

in which *, **, R²¹ and R²² each have the definitions indicated above,
can be prepared by eliminating the protective group PG¹ from a compound of the formula (VI) by methods known to the skilled person, and reacting the resultant deprotected compound in an inert solvent in the presence of a suitable base with a compound of the formula (XVII)

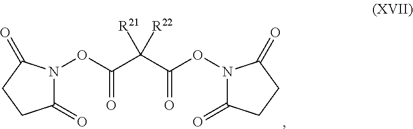

in which R²¹ and R²² each have the definitions indicated above,
to give a compound of the formula (III-D)

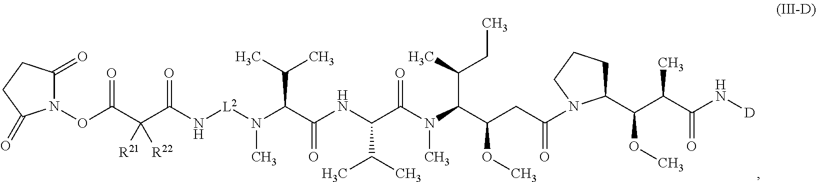

in which D, R²¹, R²² and L² each have the definitions indicated above.

The compounds of the formula (III), in which B is a group of the formula (B⁸)

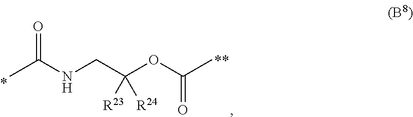

in which *, **, R²³ and R²⁴ each have the definitions indicated above,
can be prepared by reacting a compound of the formula (IX) in an inert solvent in the presence of a suitable coupling reagent and a suitable base with a compound of the formula (XVIII)

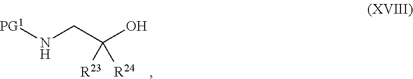

in which R²³, R²⁴ and PG¹ each have the definitions indicated above,
to give a compound of the formula (XIX)

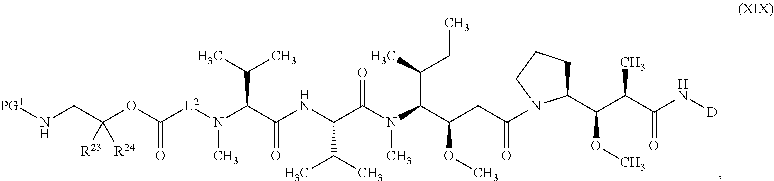

in which D, R²³, R²⁴, L² and PG¹ each have the definitions indicated above,
eliminating the protective group PG¹ from this compound by methods known to the skilled person, and subsequently reacting the deprotected compound in an inert solvent in the presence of a suitable coupling reagent and a suitable base with a compound of the formula (XX)

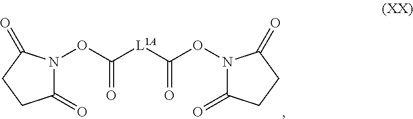

in which
L^{1A} is linear (C₁-C₁₀)-alkanediyl or is a group of the formula

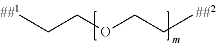

where
m is a number from 2 to 6,
##¹ marks the linkage site with the group G,
##2 marks the linkage site with the group B,
where (C₁-C₁₀)-alkanediyl may be substituted by 1 to 4 methyl substituents,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
   to give a compound of the formula (III-E)

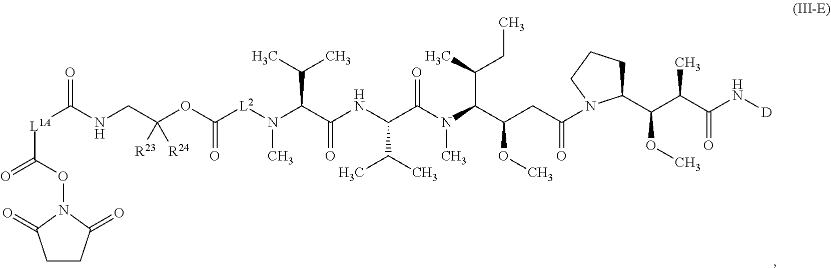

in which D, R²³, R²⁴, L^{1A} and L² each have the definitions indicated above.

The compounds of the formula (III), in which B is a group of the formula (B^{5B})

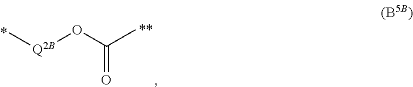

in which * and ** each have the definitions indicated above and
Q^{2B} is an N-linked 4- to 7-membered heterocycle,
can be prepared by reacting a compound of the formula (IX) in an inert solvent in the presence of a suitable base and a suitable coupling reagent with a compound of the formula (XXIV)

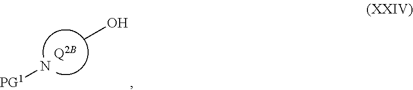

in which PG¹ and Q^{2B} each have the definitions indicated above,
to give a compound of the formula (XXV)

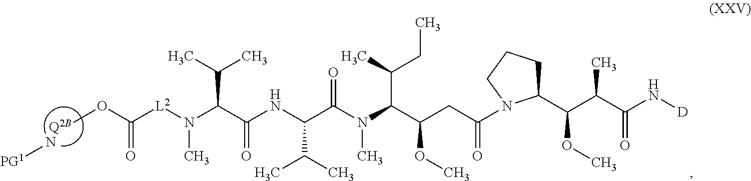

in which PG¹, Q^{2B}, D and L² have the definitions indicated above,
eliminating the protective group PG¹ from this compound by methods known to the skilled person,
and subsequently converting the deprotected compound in an inert solvent in the presence of a suitable base with a compound of the formula (XX) into a compound of the formula (III-F)

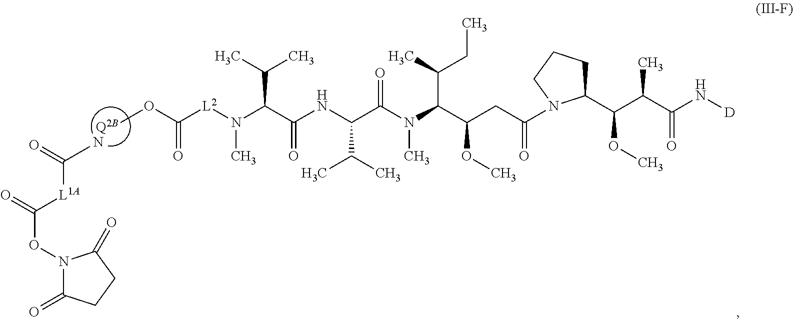

in which Q^{2B}, D, L^{1A} and L² have the definitions indicated above.

The reactions (IV)+(V)→(VI) and (IV)+(VIII)→(IX) take place in the solvents which are customary for a reductive amination and are inert under the reaction conditions, optionally in the presence of an acid and/or of a water-removing agent as catalyst. Such solvents include, for example, alcohols such as methanol, ethanol, n-propanol, isopropanol, n-butanol or tert-butanol, ethers such as tetrahydrofuran, 1,4-dioxane, 1,2-dimethoxyethane or bis(2-methoxyethyl) ether, or other solvents such as dichloromethane, 1,2-dichloroethane, N,N-dimethylformamide or else water. It is also possible to use mixtures of these solvents. As solvent it is preferred to use a 1,4-dioxane/water mixture, with addition of acetic acid or dilute hydrochloric acid as catalyst.

Reducing agents suitable for this reaction are, in particular, complex borohydrides, such as, for example, sodium borohydride, sodium cyanoborohydride, sodium triacetoxyborohydride, tetra-n-butylammonium borohydride or borane-pyridine complex. It is preferred to use sodium cyanoborohydride or borane-pyridine complex.

The reactions (IV)+(V)→(VI) and (IV)+(VIII)→(IX) take place in general in a temperature range from 0° C. to +120° C., preferably at +50° C. to +100° C. The reactions may be carried out under atmospheric, increased or reduced pressure (e.g. from 0.5 to 5 bar); it is usual to operate at atmospheric pressure.

The above-described coupling reactions (IX)+(X)→(II-C), (XII-A) or (XII-B)+(X)→(II-D-A) or (II-D-B), (IX)+(XIII)→(XIV), (IX)+(XV)→(XVI) and (XXII)+(XXIII)→(II-D) (amide formation from amine component and carboxylic acid component respectively) are carried out by standard methods of peptide chemistry [see e.g. M. Bodanszky, Principles of Peptide Synthesis, Springer-Verlag, Berlin, 1993; M. Bodanszky and A. Bodanszky, The Practice of Peptide Synthesis, Springer-Verlag, Berlin, 1984; H.-D. Jakubke and H. Jeschkeit, Aminosäuren, Peptide, Proteine, Verlag Chemie, Weinheim, 1982].

Examples of inert solvents for these coupling reactions are ethers such as diethyl ether, diisopropyl ether, tert-butyl methyl ether, tetrahydrofuran, 1,4-dioxane, 1,2-dimethoxyethane or bis(2-methoxyethyl) ether, hydrocarbons such as benzene, toluene, xylene, pentane, hexane, heptane, cyclohexane or petroleum fractions, halogenated hydrocarbons such as dichloromethane, trichloromethane, tetrachloromethane, 1,2-dichloroethane, trichloroethylene or chlorobenzene, or dipolar-aprotic solvents such as acetone, methyl ethyl ketone, acetonitrile, ethyl acetate, pyridine, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP). It is also possible to use mixtures of such solvents. Preference is given to using N,N-dimethylformamide.

Examples of suitable activating/condensing agents for these couplings include carbodiimides such as N,N′-diethyl-, N,N′-dipropyl-, N,N′-diisopropyl-, N,N′-dicyclohexylcarbodiimide (DCC) or N-(3-dimethylaminoisopropyl)-N′-ethylcarbodiimide hydrochloride (EDC), phosgene derivatives such as N,N′-carbonyldiimidazole (CDI) or isobutyl chloroformate, 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline, phosphorus compounds such as propanephosphonic anhydride, diethyl cyanophosphonate, bis(2-oxo-3-oxazolidinyl)phosphoryl chloride, benzotriazol-1-yloxytris(dimethylamino)phosphonium hexafluorophosphate or benzotriazol-1-yloxytris(pyrrolidino)phosphonium hexafluorophosphate (PyBOP), or uronium compounds such as 0-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium-tetrafluoroborate (TCTU), optionally in combination with further auxiliaries such as 1-hydroxybenzotriazole (HOBt) or N-hydroxysuccinimide (HOSu), and also, as bases, alkali metal carbonates, e.g. sodium or potassium carbonate, or tertiary amine bases such as triethylamine, N-methylmorpholine, N-methylpiperidine, N,N-diisopropylethylamine, pyridine or 4-N,N-dimethylaminopyridine.

In the context of the present invention, as activating/condensing agents for such coupling reactions, it is preferred to use N-(3-dimethylaminoisopropyl)-N′-ethylcarbodiimide hydrochloride (EDC) in combination with 1-hydroxybenzotriazole (HOBt) and N,N-diisopropylethylamine, or O-(7-azabenzotriazol-1-yl)-N,N,N′,N′-tetramethyluronium hexafluorophosphate (HATU) likewise in conjunction with N,N-diisopropylethylamine

The coupling reactions (IX)+(X)→(II-C), (XII-A) or (XII-B)+(X)→(II-D-A) or (II-D-B), (IX)+(XIII)→(XIV), (IX)+(XV)→(XVI) and (XXII)+(XXIII)→(II-D) are carried out in general in a temperature range from −20° C. to +60° C., preferably at 0° C. to +40° C. The reactions may take place under atmospheric, at increased or at reduced pressure (e.g. from 0.5 to 5 bar); it is usual to operate under atmospheric pressure.

The esterifications (IX)+(XVIII)→(XII) and (IX)+(XI-A) or (XI-B)→(XII-A) or (XII-B), (IX)+(XXIV)→(XXV) and also (IX)+(XXI)→(XXII) take place in analogy to the above-described amide coupling reactions. These reactions take place preferably in dichloromethane, using N-(3-dimethylaminoisopropyl)-N′-ethylcarbodiimide hydrochloride (EDC) and 4-dimethylaminopyridine at a temperature of +50° C. to 100° C. under atmospheric pressure.

The functional groups optionally present in the compounds such as amino, hydroxyl and carboxyl groups in particular may also be present in a temporarily protected form during the above-described process steps, if useful or necessary. In these cases, such protective groups are introduced and removed in accordance with customary methods known from peptide chemistry [see, for example, T. W. Greene and P. G. M. Wuts, Protective Groups in Organic Synthesis, Wiley, New York, 1999; M. Bodanszky and A. Bodanszky, The Practice of Peptide Synthesis, Springer-Verlag, Berlin, 1984]. Where two or more protected groups are present, they can be liberated again optionally simultaneously in a one-pot reaction, or else liberated again in separate reaction steps.

As an amino-protective group PG¹ it is preferred to use tert-butoxycarbonyl (Boc), benzyloxycarbonyl (Z) or (9H-fluoren-9-ylmethoxy)carbonyl (Fmoc); for a hydroxyl or carboxyl function it is preferred to use tert-butyl or benzyl as protective group PG². The elimination of a tert-butyl or tert-butoxycarbonyl group is typically accomplished by treatment with a strong acid, such as hydrogen chloride, hydrogen bromide or trifluoroacetic acid, in an inert solvent such as diethyl ether, 1,4-dioxane, dichloromethane or acetic acid; this reaction may optionally also be carried out without addition of an inert solvent. In the case of benzyl or benzyloxycarbonyl as protective group, this group is removed preferably by hydrogenolysis in the presence of a suitable palladium catalyst, such as palladium on activated carbon, for example. The (9H-fluoren-9-ylmethoxy)carbonyl group is generally eliminated using a secondary amine base such as diethylamine or piperidine.

The reaction (VI)→(II-A) takes place in a solvent which is inert under the reaction conditions, such as, for example, ethers such as tetrahydrofuran, 1,4-dioxane, 1,2-dimethoxyethane or bis(2-methoxyethyl) ether, alcohols such as methanol, ethanol, isopropanol, n-butanol or tert-butanol, or dipolar-aprotic solvents such as acetone, methyl ethyl ketone, acetonitrile, ethyl acetate, pyridine, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP) or water. It is also possible to use mixtures of such solvents. Preference is given to using a mixture of 1,4-dioxane and water.

Suitable bases for the reaction (VI)→(II-A) are, for example, alkali metal carbonates such as potassium carbonate, sodium carbonate or lithium carbonate, alkali metal hydrogencarbonates such as sodium or potassium hydrogencarbonate or alkali metal alkoxides such as sodium methoxide, sodium ethoxide or potassium tert-butoxide. It is preferred to use sodium hydrogencarbonate.

The reaction (VI)→(II-A) takes place in a temperature range from 0° C. to +50° C., preferably at +10° C. to +30° C. The reaction may take place under atmospheric, under elevated or under reduced pressure (e.g. from 0.5 to 5 bar); it is usual to operate under atmospheric pressure.

The reaction (VI)+(VII)→(II-B) takes place in a solvent which is inert under the reaction conditions, such as, for example, ethers such as tetrahydrofuran, 1,4-dioxane, 1,2-dimethoxyethane or bis(2-methoxyethyl) ether, alcohols such as methanol, ethanol, isopropanol, n-butanol or tert-butanol, or dipolar-aprotic solvents such as acetone, methyl ethyl ketone, acetonitrile, ethyl acetate, pyridine, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP) or water. It is also possible to use mixtures of such solvents. Preference is given to using DMF.

Suitable bases for the reaction (VI)+(VII)→(II-B) are, for example, tertiary amine bases such as triethylamine, N-methylmorpholine, N-methylpiperidine, N,N-diisopropylethylamine, pyridine or 4-N,N-dimethylaminopyridine. Preference is given to using N,N-diisopropylethylamine

The reaction (VI)+(VII)→(II-B) takes place in a temperature range from 0° C. to +50° C., preferably at +10° C. to +30° C. The reaction may take place under atmospheric, under elevated or under reduced pressure (e.g. from 0.5 to 5 bar); it is usual to operate under atmospheric pressure.

The reactions (IX)→(III-A), (XIV)→(III-B) and (XVI)→(III-C) and also (VI)+(XVII)→(III-D), (XIX)+(XX)→(III-E) and (XXV)+(XX)→(III-F) take place in a solvent which is inert under the reaction conditions. Examples of suitable solvents are ethers such as diethyl ether, diisopropyl ether, tert-butyl methyl ether, tetrahydrofuran, 1,4-dioxane, 1,2-dimethoxyethane or bis(2-methoxyethyl) ether, hydrocarbons such as benzene, toluene, xylene, pentane, hexane, heptane, cyclohexane or petroleum fractions, halogenated hydrocarbons such as dichloromethane, trichloromethane, tetrachloromethane, 1,2-dichloroethane, trichloroethylene or chlorobenzene, or dipolar-aprotic solvents such as acetone, methyl ethyl ketone, acetonitrile, ethyl acetate, pyridine, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP). It is also possible to use mixtures of such solvents. Preference is given to using N,N-dimethylformamide.

Suitable bases for these reactions are, for example, tertiary amines such as triethylamine, N-methylmorpholine, N-methylpiperidine, N,N-diisopropylethylamine, pyridine or 4-N,N-dimethylaminopyridine. Preference is given to using N,N-diisopropylethylamine, optionally with addition of 4-N,N-dimethylaminopyridine.

The reactions (IX)→(III-A), (XIV)→(III-B) and (XVI)→(III-C) and also (VI)+(XVII)→(III-D) and (XIX)+(XX)→(III-E) take place in a temperature range from 0° C. to +50° C., preferably at +10° C. to +30° C. The reaction may take place under atmospheric, under elevated or under reduced pressure (e.g. from 0.5 to 5 bar); it is usual to operate under atmospheric pressure.

The compounds of the formulae (II) and (III) are sub-quantities of the compounds of the formulae (IIa) and (IIIa) respectively, where R³⁵ is methyl. The preparation of the compounds (IIa) and (IIIa) takes place in analogy to the preparation of the compound of the formulae (II) and (III) as described above.

The above-described processes are illustrated by way of example by the following synthesis schemes (Scheme 3 to 13, 18):

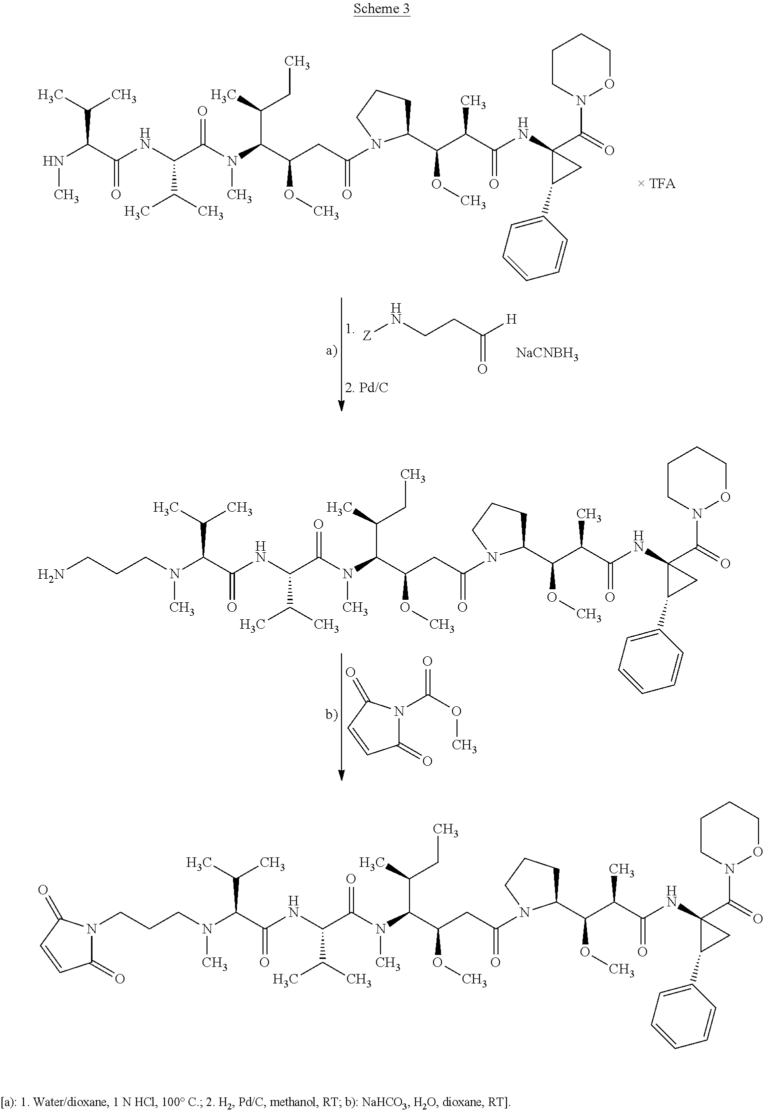

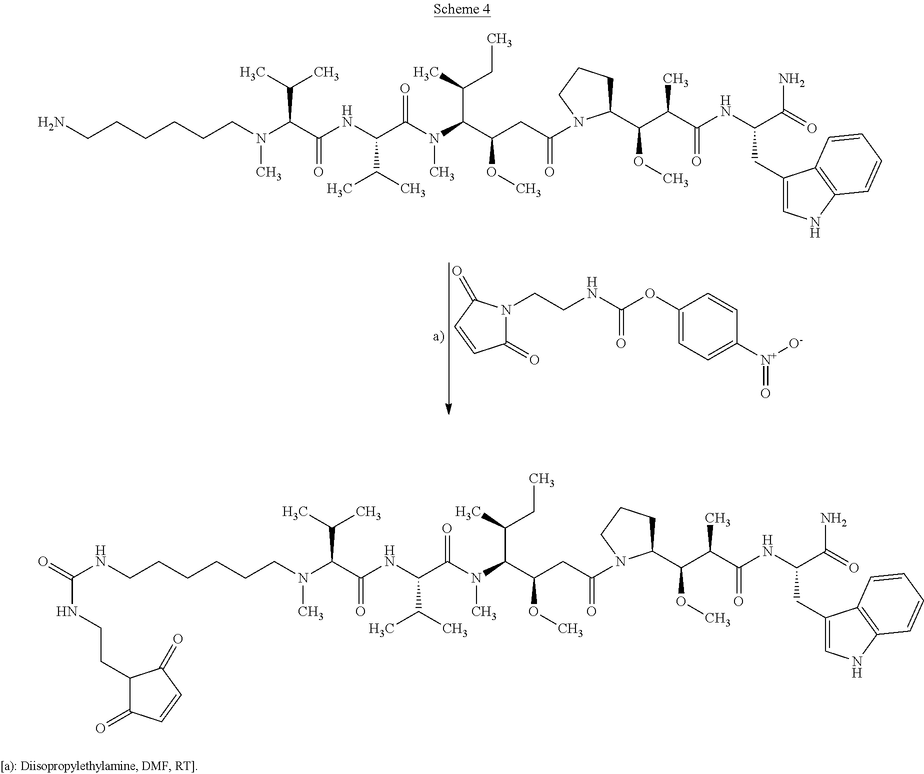 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCCCCN)C(C)C ; O=C(NCCN1C(=O)C=CC1=O)Oc1ccc([N+](=O)[O-])cc1 ; CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCCCCNC(=O)NCCC1C(=O)C=CC1=O)C(C)C]

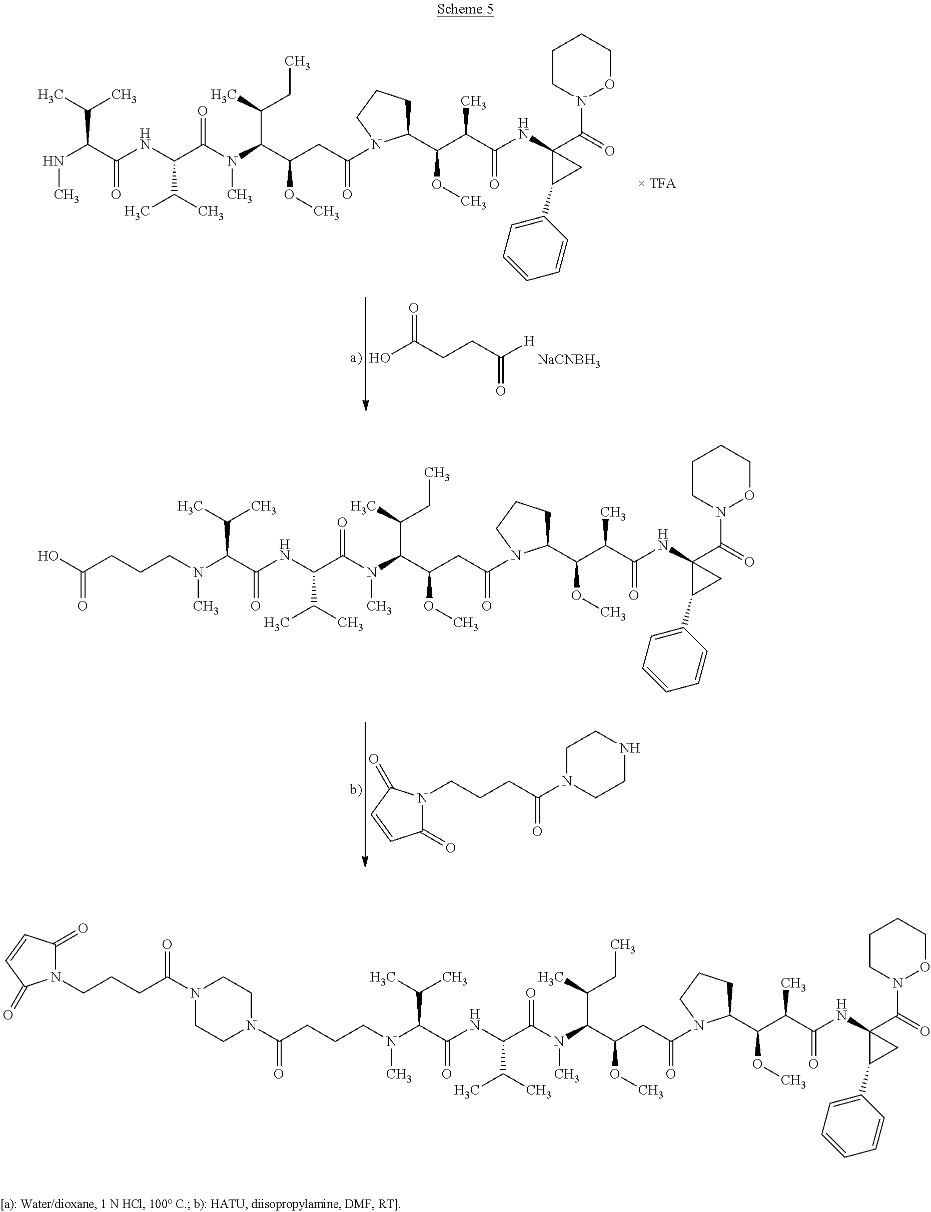

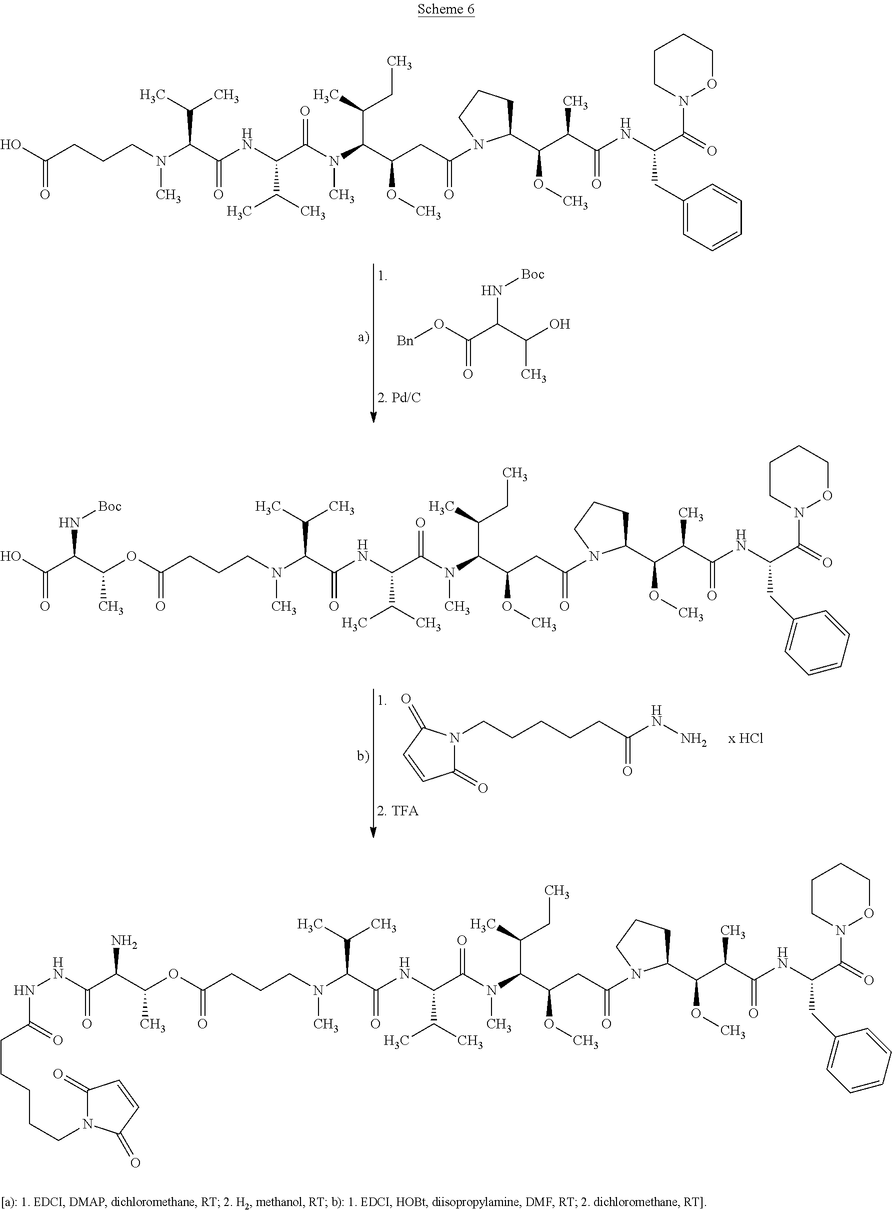

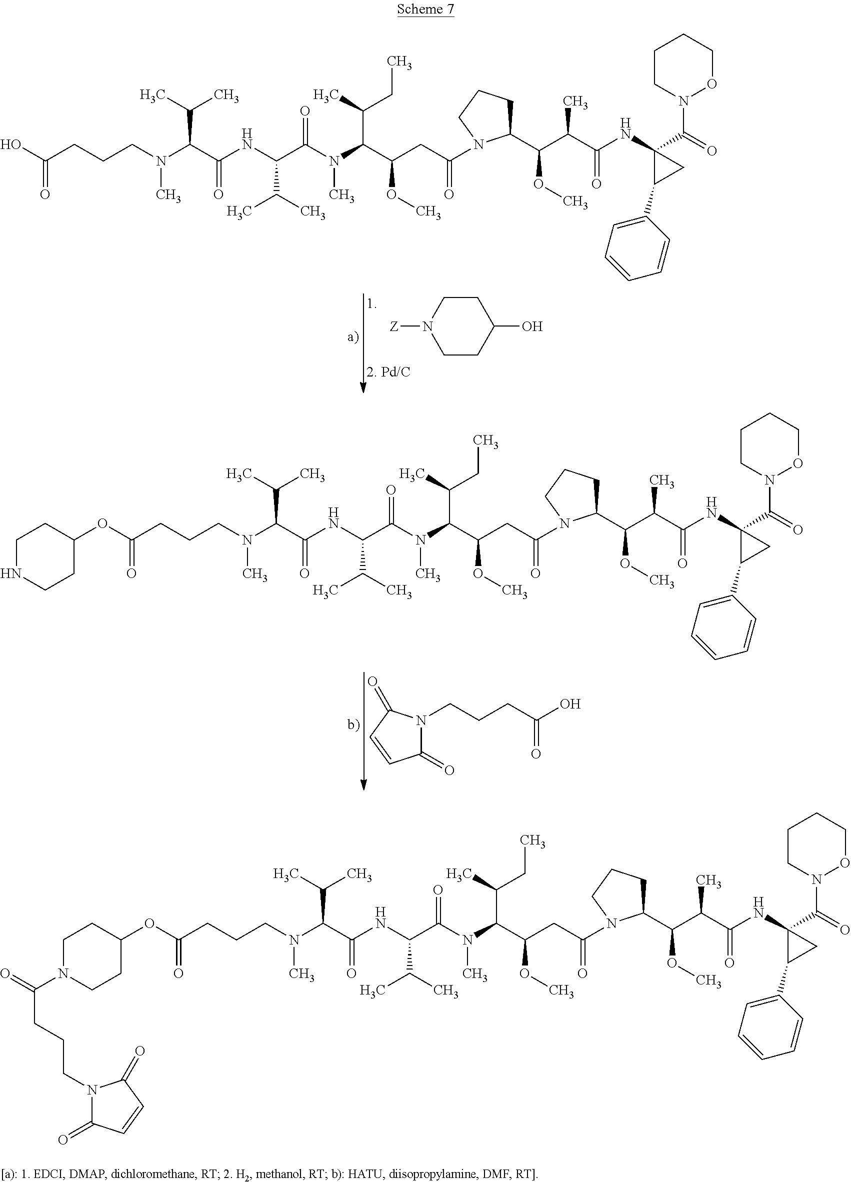

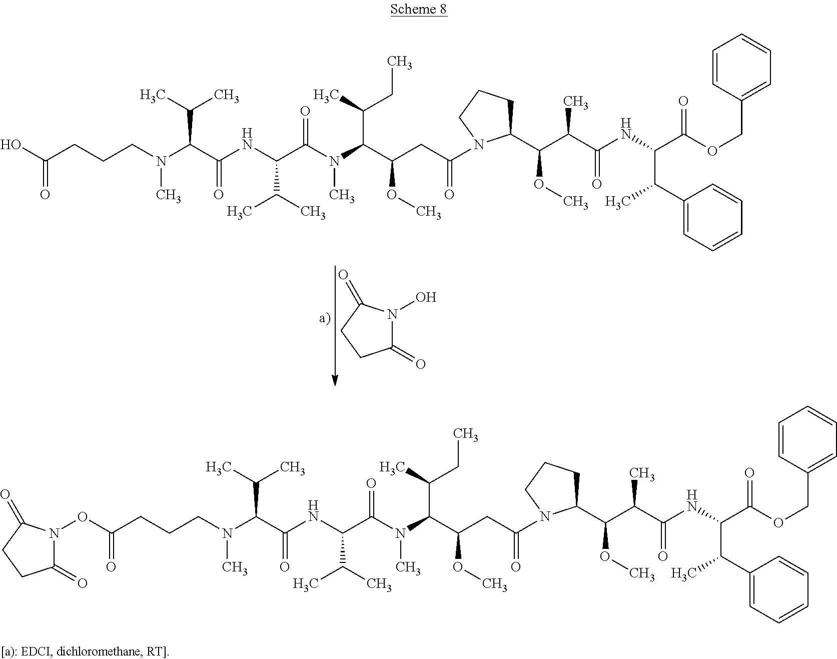 [SMILES: CC[C@H](C)[C@@H]([C@@H](CC(=O)N1CCC[C@H]1[C@H](OC)[C@@H](C)C(=O)N[C@H](C(=O)OCc1ccccc1)[C@@H](C)c1ccccc1)OC)N(C)C(=O)[C@@H](NC(=O)[C@H](C(C)C)N(C)CCCC(=O)O)C(C)C ; O=C1CCC(=O)N1O ; CC[C@H](C)[C@@H]([C@@H](CC(=O)N1CCC[C@H]1[C@H](OC)[C@@H](C)C(=O)N[C@H](C(=O)OCc1ccccc1)[C@@H](C)c1ccccc1)OC)N(C)C(=O)[C@@H](NC(=O)[C@H](C(C)C)N(C)CCCC(=O)ON1C(=O)CCC1=O)C(C)C]

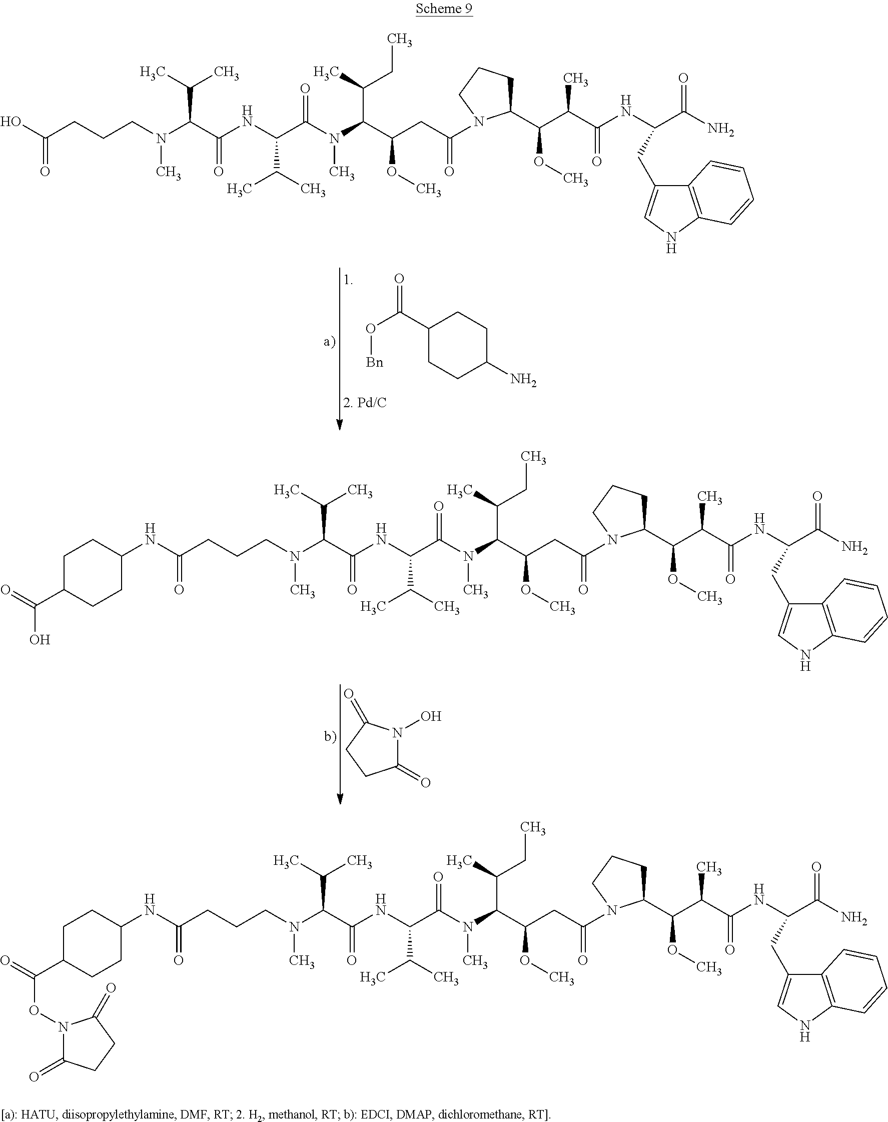

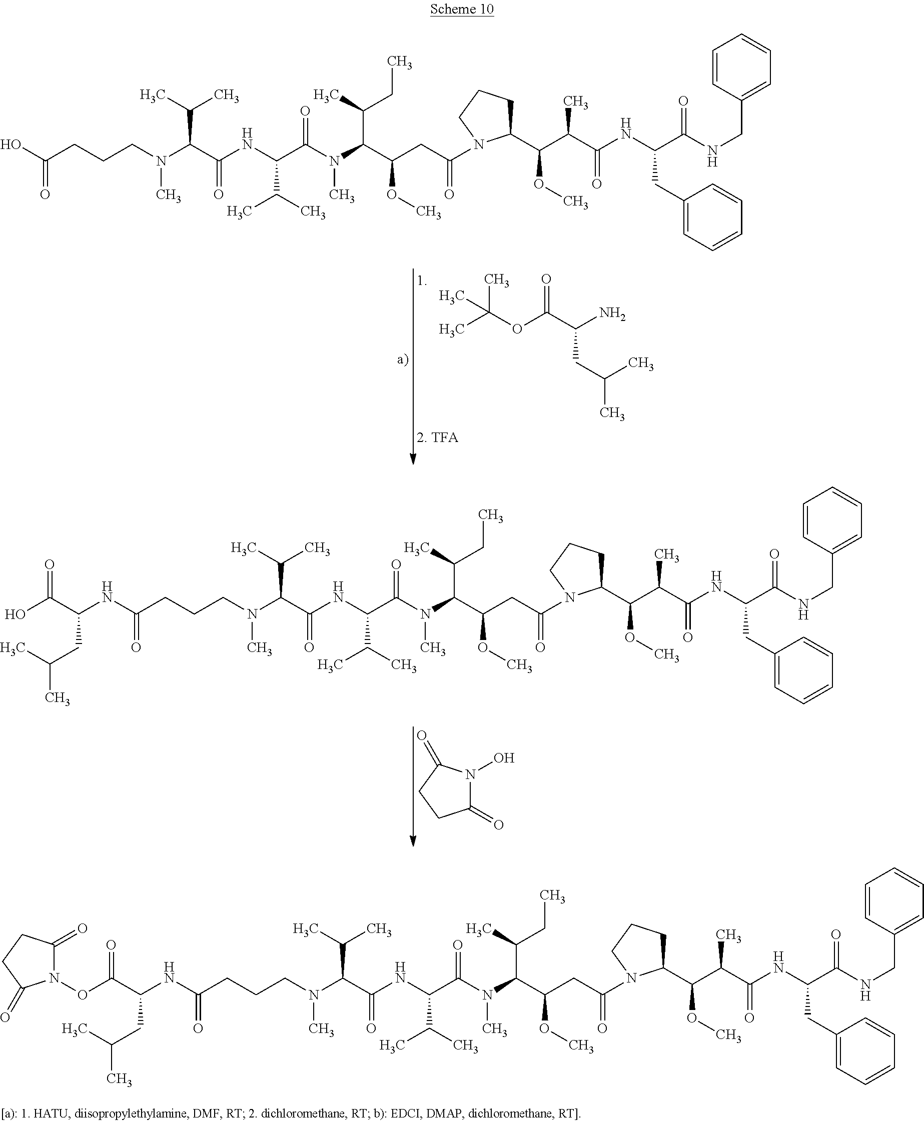

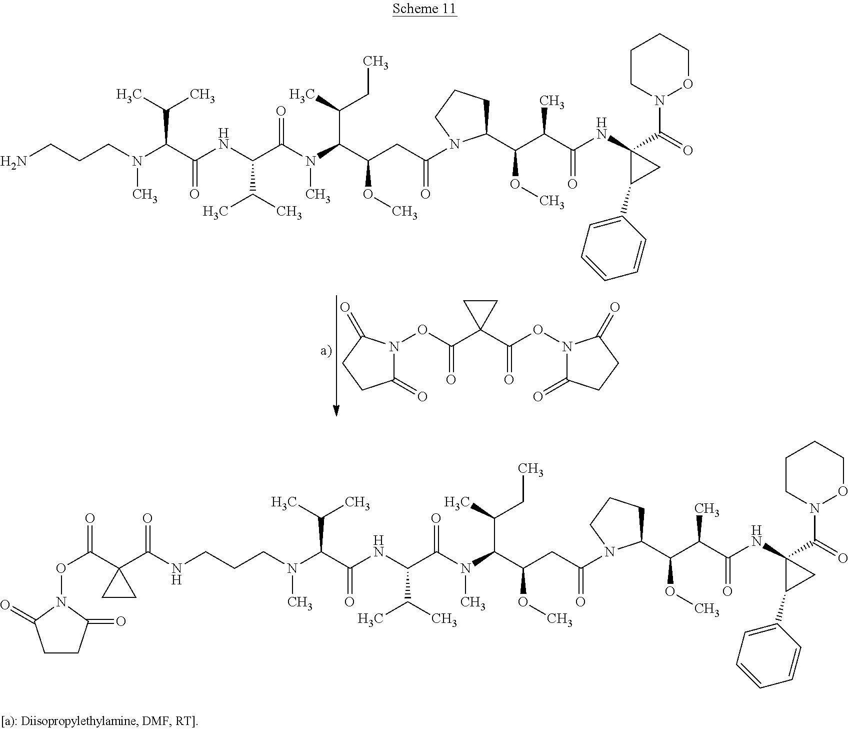

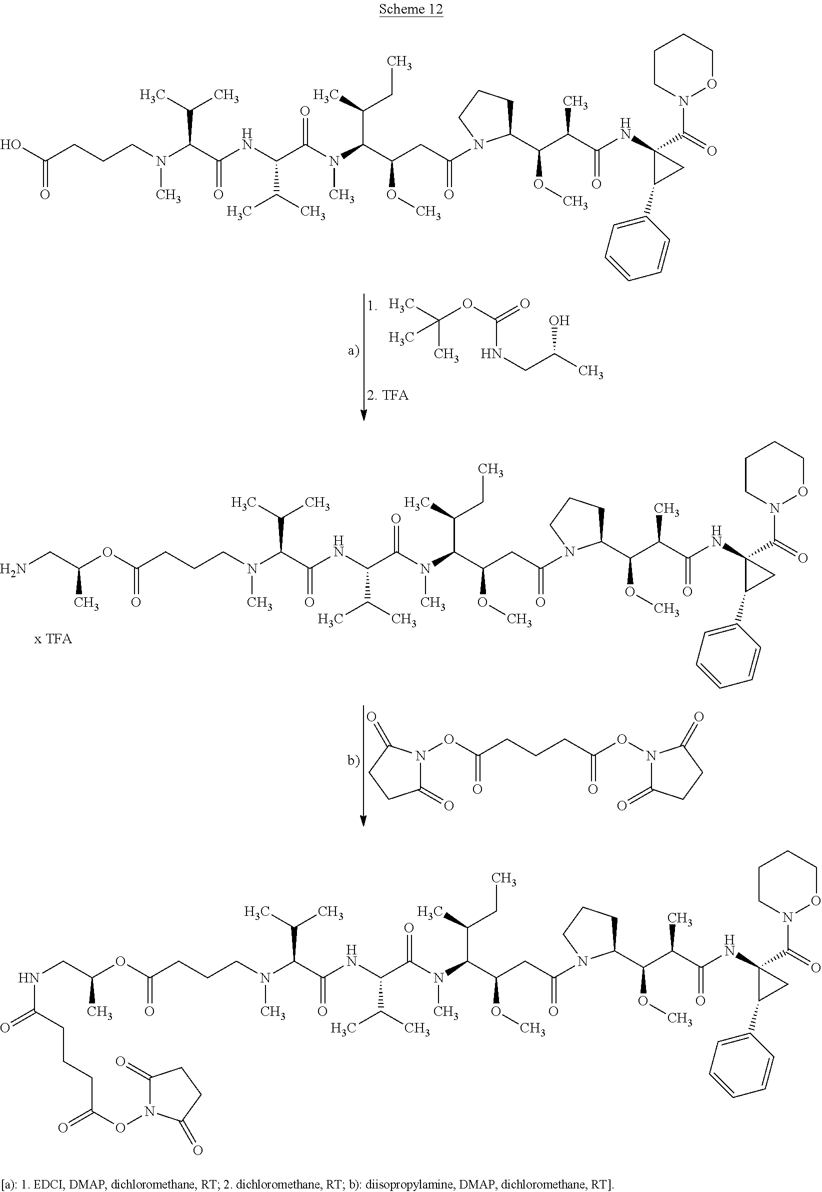

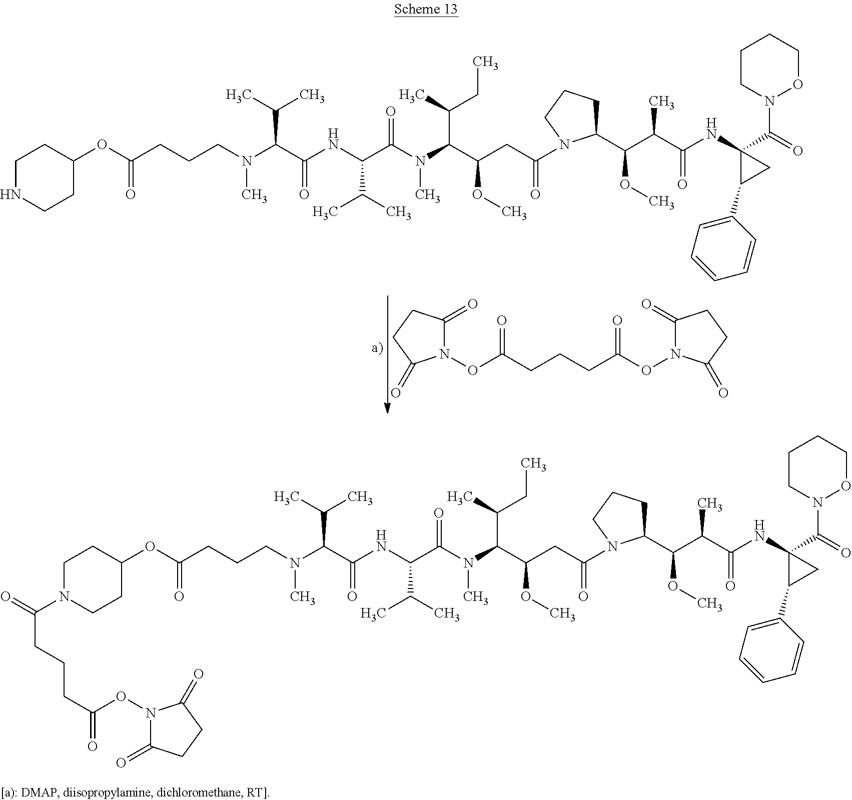

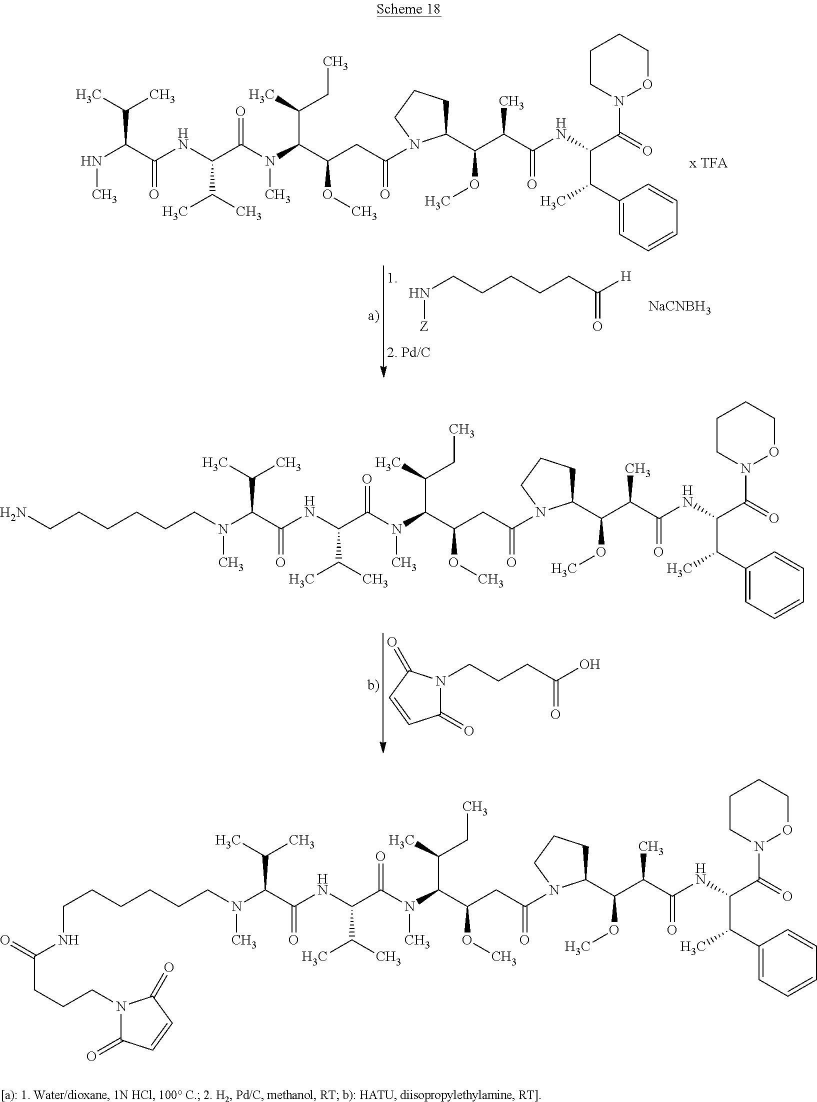

The compounds of the formula (IV) can be prepared from commercially available amino acid building blocks or those known from the literature (see, for example, Pettit et al., Synthesis 1996, 719; Shioiri et al., Tetrahedron Lett. 1991, 32, 931; Shioiri et al., Tetrahedron 1993, 49, 1913; Koga et al., Tetrahedron Lett. 1991, 32, 2395; Vidal et al., Tetrahedron 2004, 60, 9715; Poncet et al., Tetrahedron 1994, 50, 5345. Pettit et al., J. Org. Chem. 1994, 59, 1796) in analogy to processes known from the literature, in accordance with customary methods of peptide chemistry, and as described in the present experimental section. The synthesis schemes below (Scheme 14 to 16) illustrate the preparation by way of example.

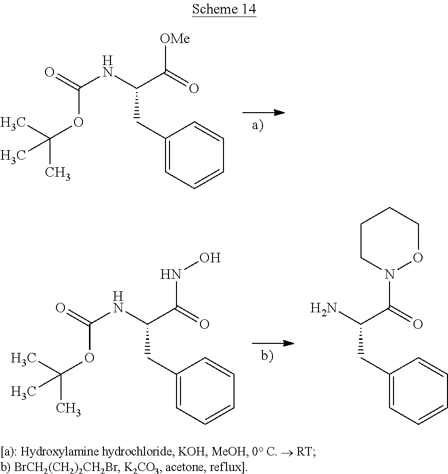 [SMILES: COC(=O)[C@H](Cc1ccccc1)NC(=O)OC(C)(C)C ; CC(C)(C)OC(=O)N[C@@H](Cc1ccccc1)C(=O)NO ; N[C@@H](Cc1ccccc1)C(=O)N1CCCCO1]

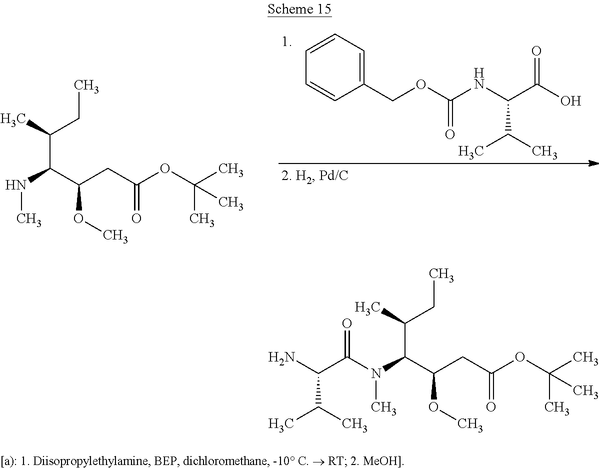 [SMILES: CC[C@H](C)[C@H](NC)[C@@H](CC(=O)OC(C)(C)C)OC ; CC(C)[C@H](NC(=O)OCc1ccccc1)C(=O)O ; CC[C@H](C)[C@@H]([C@@H](CC(=O)OC(C)(C)C)OC)N(C)C(=O)[C@@H](N)C(C)C]

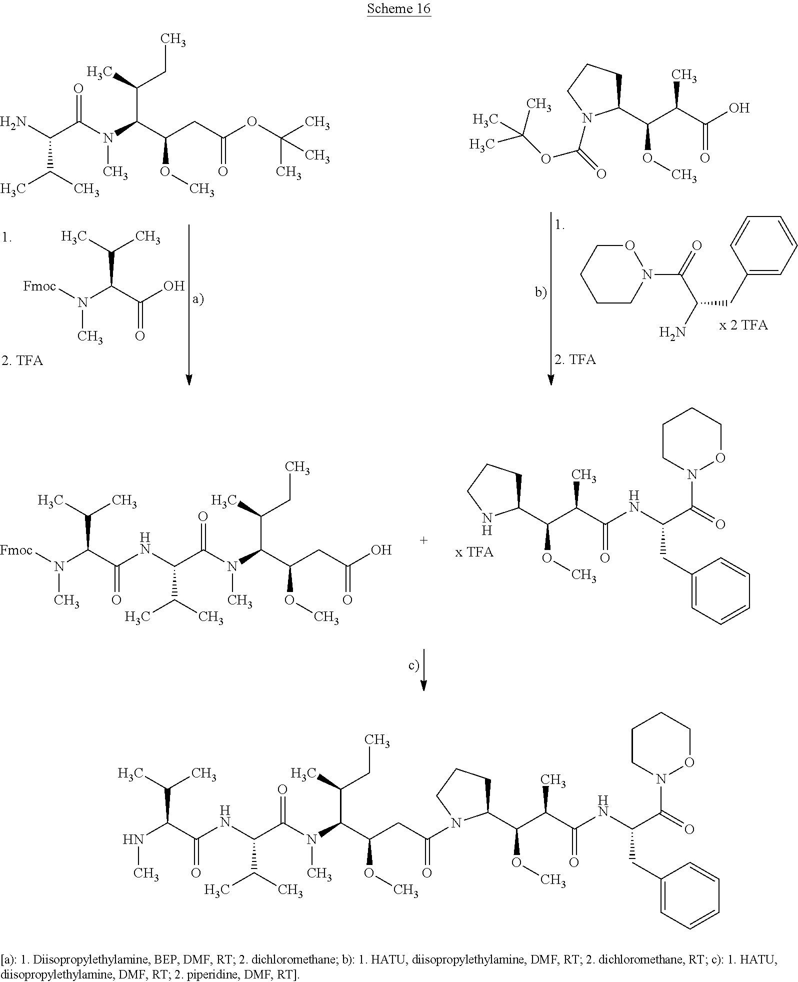

The compounds of the formulae (XI), (XIII), (XV), (XVII) and (XXI), including, where appropriate, chiral or diastereomeric forms thereof, are available commercially or are described as such in the literature, or they can be prepared by routes that are obvious to the skilled person, in analogy to methods published in the literature. Numerous comprehensive instructions and also literature information on the preparation of the starting materials are also given in the experimental section, in the section relating to the preparation of the starting compounds and intermediates.

The compounds of the formulae (V), (VII), (VIII), (X), (XVIII), (XX) and (XXIII), including, where appropriate, chiral or diastereomeric forms thereof, are known from the literature, or can be prepared by routes which are obvious to the skilled person, in analogy to methods published in the literature. Numerous comprehensive instructions and also literature information on the preparation of the starting materials are also given in the experimental section, in the section relating to the preparation of the starting compounds and intermediates.

Alternatively, individual steps of the preparation sequence may be carried out in a different order or with other protective group combinations. This approach is illustrated by way of example in the synthesis schemes below (Scheme 17, 19, 20 and 21).

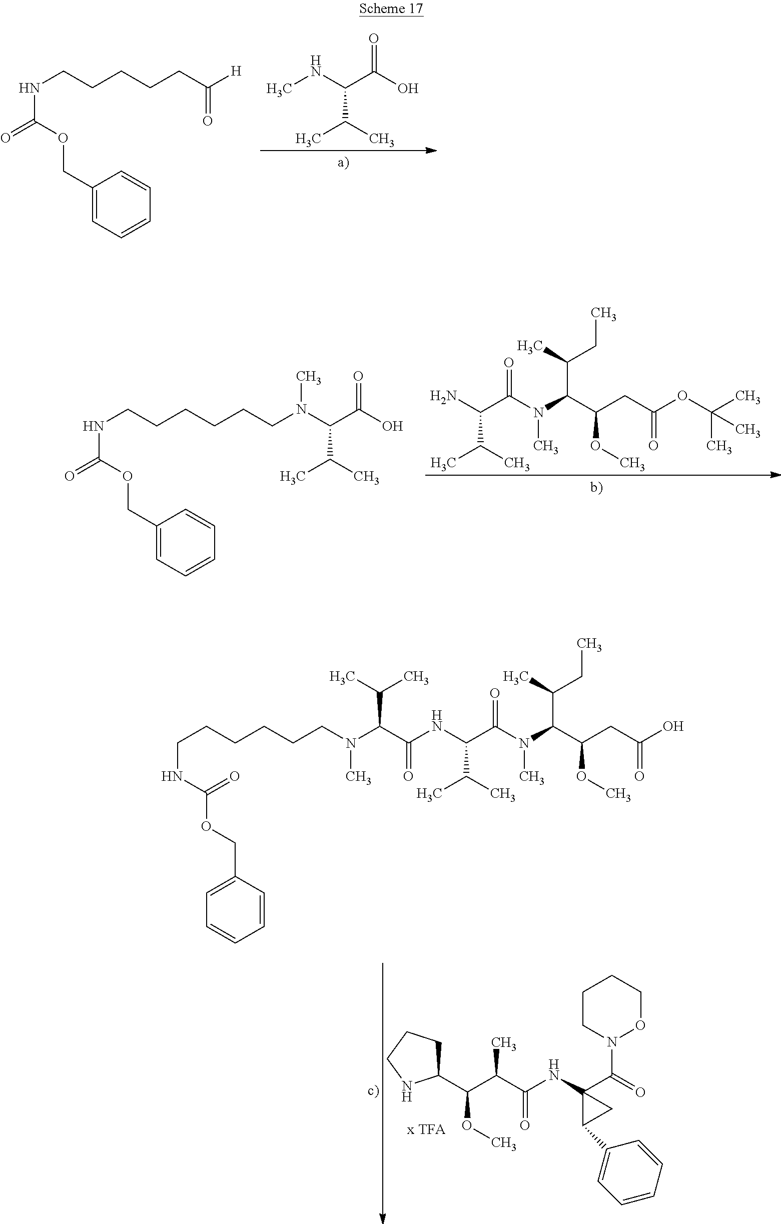

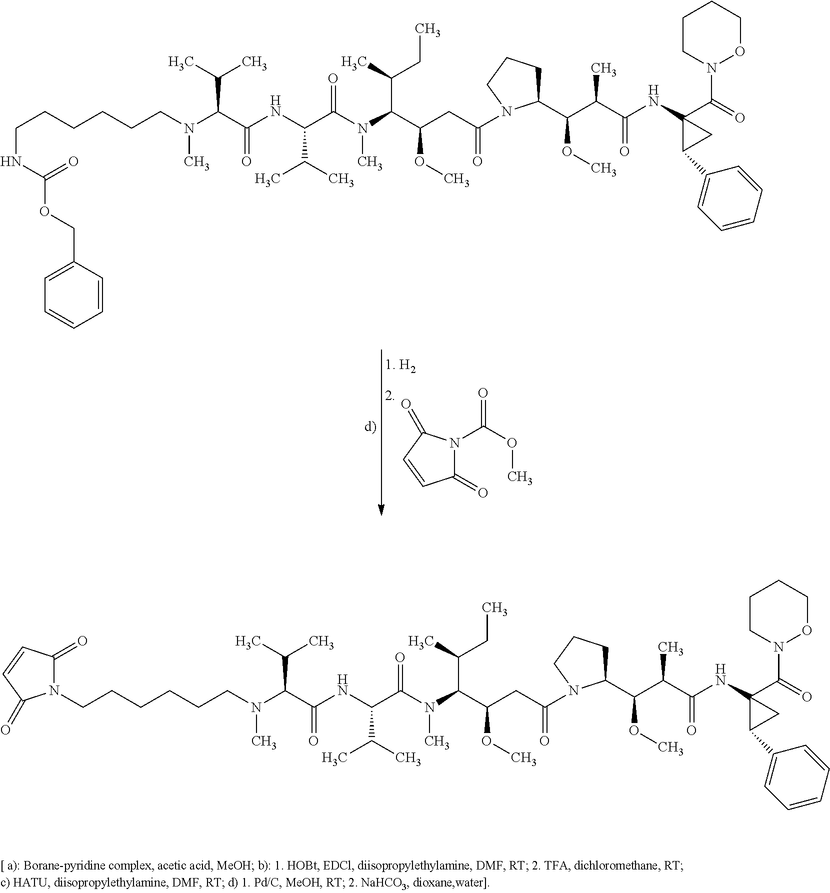

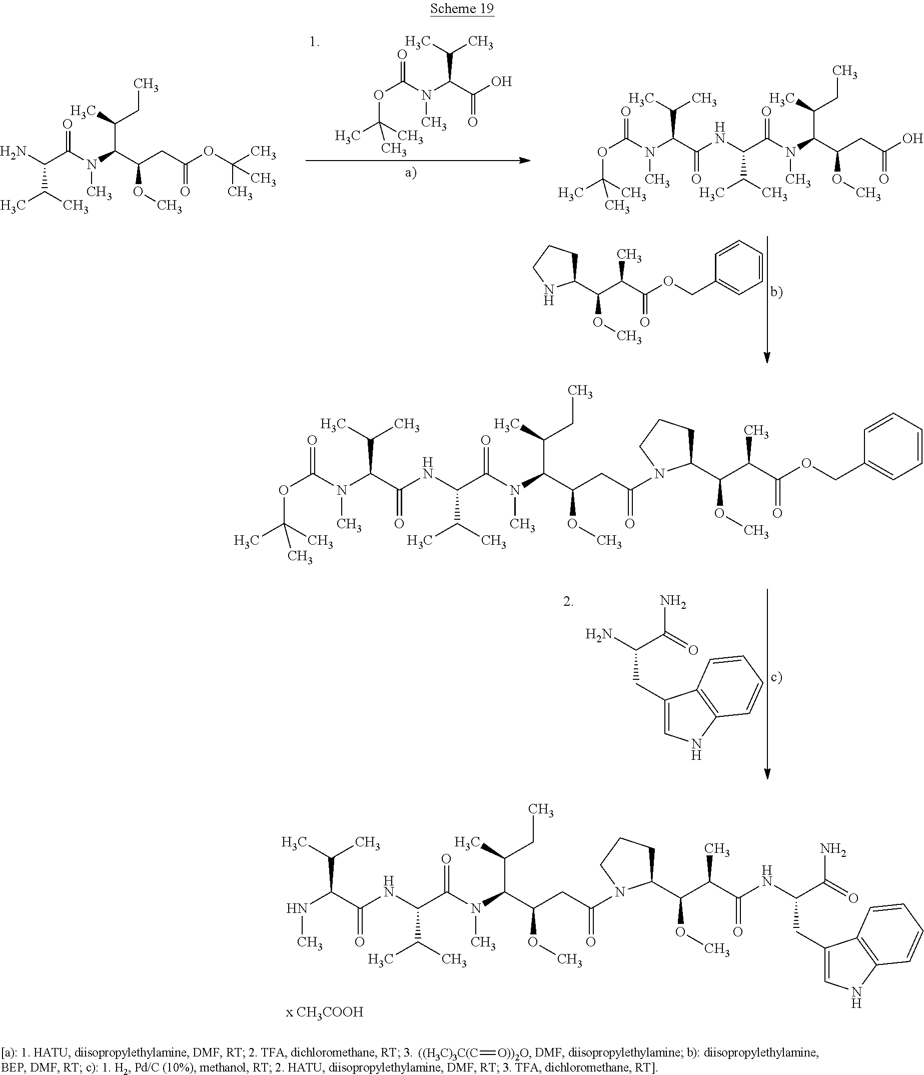

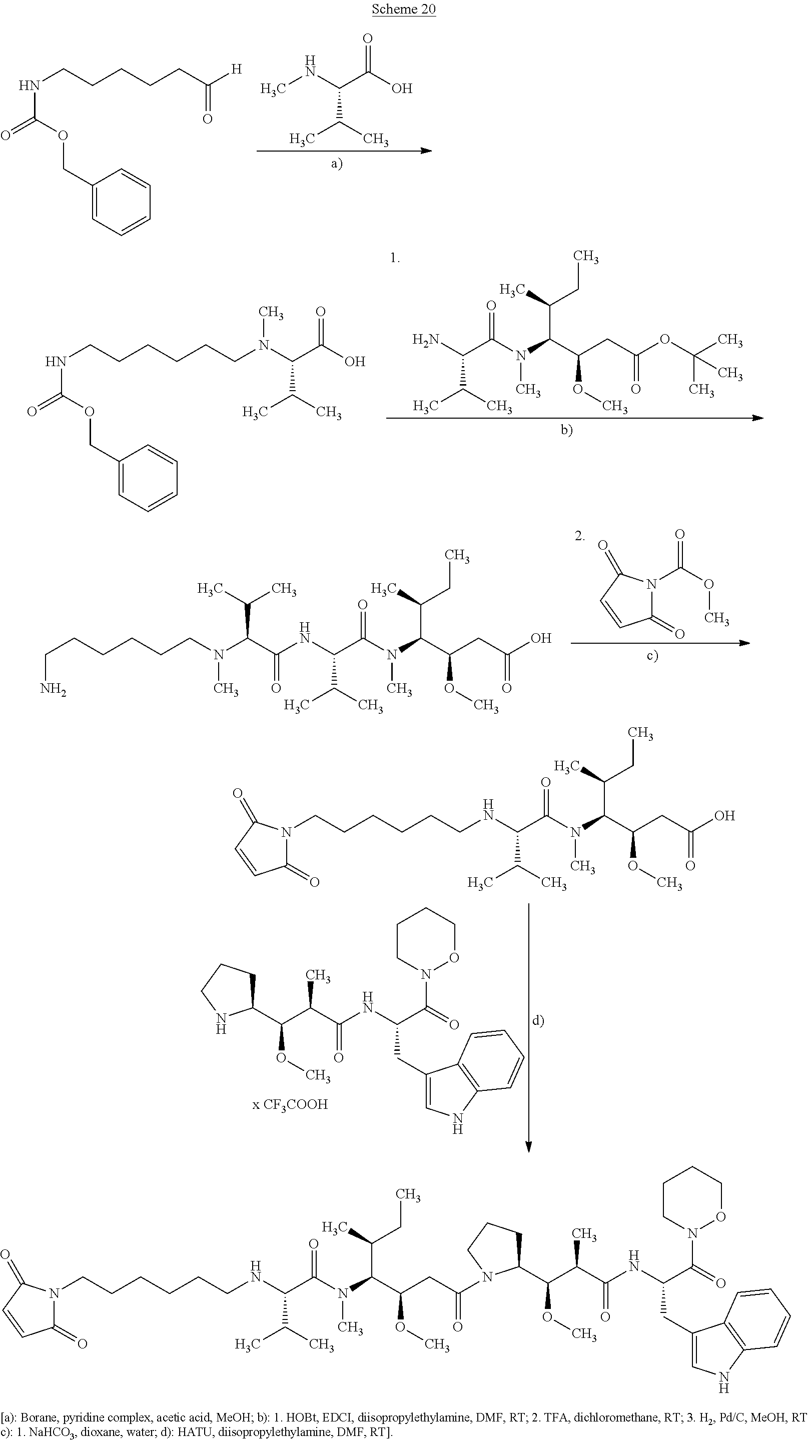

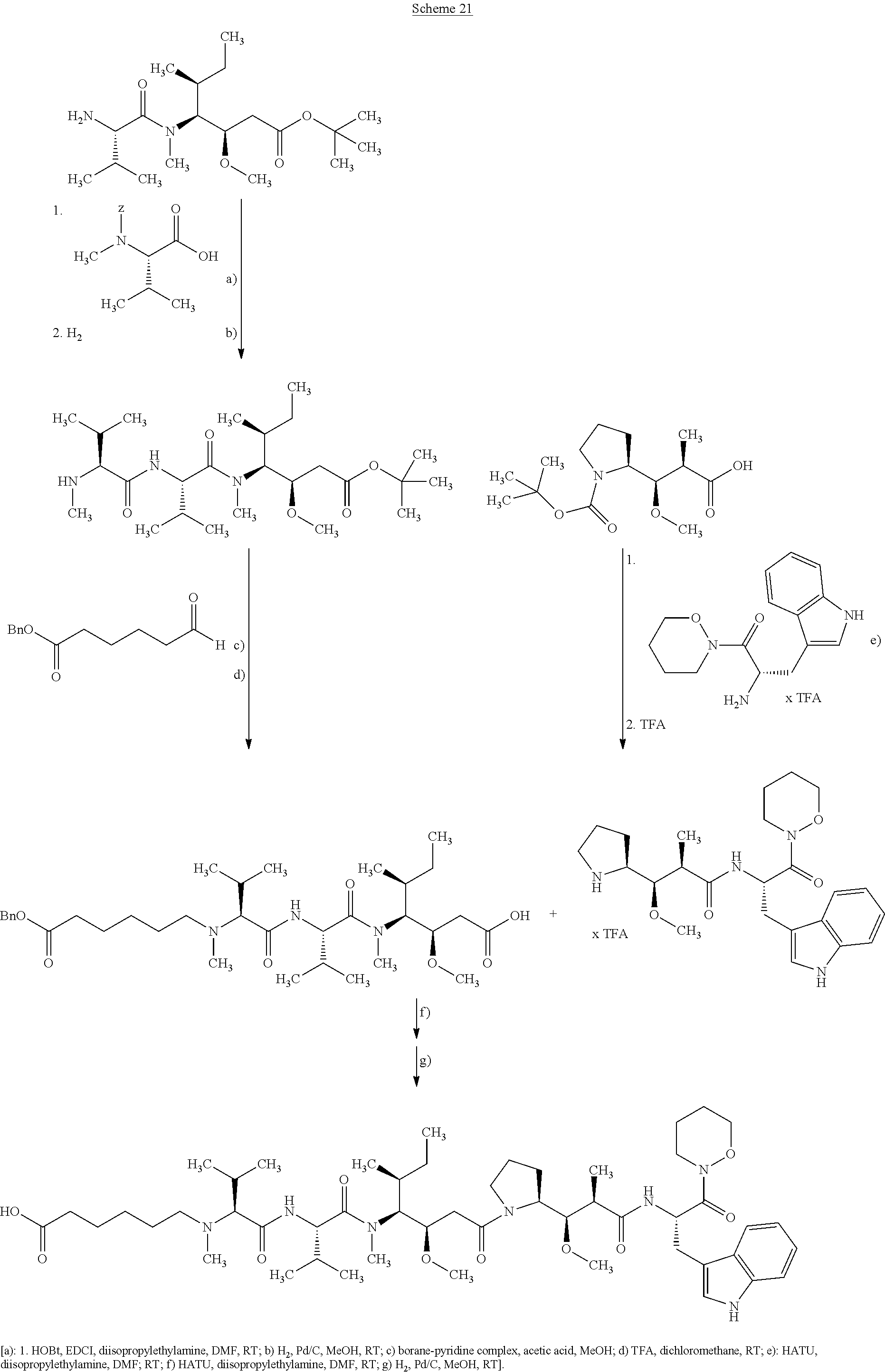

The FGFR2 cancer target molecule of the binder of the present invention is known to the skilled person. The full-length FGFR2 is identified as FGFR2 alpha (SEQ ID NO: 1), while the isoform lacking the D1 domain is identified as FGFR2 beta (SEQ ID NO: 2) (see FIG. 1). Alternative splicing in domain 3 leads to two different variants, namely FGFR2 IIIb, which is encoded by the exons 7 and 8, and FGFR2 IIIc, which is encoded by the exons 7 and 9 (see FIG. 1).

In one embodiment of the invention the binder binds—preferably specifically—to FGFR2. In a further subject of the invention, the binder binds—preferably specifically—to the extracellular domain of the target molecule FGFR2 (see FIG. 1).

In a further embodiment of the invention, the binder binds—preferably specifically—to one or more forms of the human FGFR2 polypeptide. In a further subject of the invention, the binder binds—preferably specifically—to all isoforms and splice variants of FGFR2. In the text below, the concept of different “forms” of FGFR2 includes, though is not limited to, different isoforms, different splice variants, different glycoforms or FGFR2 polypeptides which undergo different translational and post-translational modifications.

In a further embodiment of the invention, the binder binds—preferably specifically—to the N-terminal domains of the cancer target molecule FGFR2. In a further subject of the invention, the binder binds—preferably specifically—to the extracellular N-terminal epitope (¹RPSFSLVEDTTLEPE¹⁵) of FGFR2 (SEQ ID NO: 23).

In a further embodiment of the invention, the binder also binds preferably specifically to the FGFR2 of different species. The effect of this is that the conjugates of the invention can more easily, pharmacologically, be investigated in these species. Preferred species are rodents, more particularly mice or rats, but also dogs, pigs and non-human primates.

In one preferred embodiment the binder, after binding to FGFR2 on the target cell, is internalized by the target cell as a result of the binding. The effect of this is that the binder-drug conjugate, which may be an immunoconjugate or an ADC, is taken up by the target cell.

In one embodiment the binder is a binding protein. In one preferred embodiment the binder is an antibody, an antigen-binding antibody fragment, a multispecific antibody or an antibody mimetic.

Preferred antibody mimetics are affibodies, adnectins, anticalins, DARPins, avimers, or nanobodies. Preferred multispecific antibodies are bispecific and trispecific antibodies.

In one preferred embodiment the binder is an antibody or an antigen-binding antibody fragment, more preferably an isolated antibody or an isolated antigen-binding antibody fragment.

Preferred antigen-binding antibody fragments are Fab, Fab′, F(ab′)₂ and Fv fragments, diabodies, DAbs, linear antibodies and scFv. Particularly preferred are Fab, diabodies and scFv.

In one particularly preferred embodiment the binder is an antibody. Particularly preferred are monoclonal antibodies or antigen-binding antibody fragments thereof. Further particularly preferred are human, humanized or chimeric antibodies or antigen-binding antibody fragments thereof.

In one preferred embodiment, the antibody or the antigen-binding fragment comprises the amino acid sequence of the CDR sequences of the variable light and heavy chain of the antibody M048-D01-hIgG1.

In a further preferred embodiment, the antibody or the antigen-binding fragment comprises the amino acid sequence of the CDR sequences of the variable light and heavy chain of the antibody M048-D01-hIgG1 represented in SEQ ID NO: 15 (H-CDR1), SEQ ID NO: 16 (H-CDR2), SEQ ID NO: 17 (H-CDR3), SEQ ID NO: 18 (L-CDR1), SEQ ID NO: 19 (L-CDR2) and SEQ ID NO: 20 (L-CDR3).

In a further preferred embodiment, the antibody or the antigen-binding fragment comprises the amino acid sequence of the variable light and heavy chains of the antibody M048-D01-hIgG1 or M048-D01-hIgG1-b.

In a further preferred embodiment, the antibody or the antigen-binding fragment comprises the amino acid sequence of the variable light and heavy chains of the antibody M048-D01-hIgG1, represented in SEQ ID NO: 12 (V1) and SEQ ID NO: 11 (Vh), or of the variable light and heavy chains of the antibody M048-D01-hIgG1-b, represented in SEQ ID NO: 14 (V1) and SEQ ID NO: 13 (Vh).

In a further particularly preferred embodiment, the antibody or the antigen-binding fragment comprises the amino acid sequence of the variable light and heavy chain of the antibody M048-D01-hIgG1-b represented in SEQ ID NO: 14 (Vl) and SEQ ID NO: 13 (Vh).

In a further particularly preferred embodiment, the antibody comprises the amino acid sequence of the light and heavy chain of the antibody M048-D01-hIgG1-b represented in SEQ ID NO: 9 (light chain) and SEQ ID NO: 10 (heavy chain).

In a further particularly preferred embodiment, the antibody comprises the amino acid sequence of the light and heavy chain of the antibody M048-D01-hIgG1 represented in SEQ ID NO: 7 (light chain) and SEQ ID NO: 8 (heavy chain).

Examples of further FGFR2 antibodies are the GAL-FR21-mIgG1 (SEQ ID NO: 3 and SEQ ID NO: 4) and GAL-FR22-mIgG2a (SEQ ID NO: 5 and SEQ ID NO: 6) antibodies that are described in this invention. The two last-mentioned antibodies were constructed on the basis of WO2010/054265 from the variable regions, described therein, of the light (Vl) and heavy (Vh) chains of the antibodies GAL-FR21 (SEQ ID NO: 1 and SEQ ID NO: 4 from WO2010/054265) and GAL-FR22 (SEQ ID NO: 7 and SEQ ID NO: 8 from WO2010/054265), with the variable regions of GAL-FR21 having been reformatted into an mIgG1 format, while the variable regions of GAL-FR22 were reformatted into an mIgG2a format.

Antibodies or antigen-binding antibody fragments which bind cancer target molecules may be prepared by a person of ordinary skill in the art using known processes, such as, for example, chemical synthesis or recombinant expression. Binders for cancer target molecules may be acquired commercially or may be prepared by a person of ordinary skill in the art using known processes, such as, for example, chemical synthesis or recombinant expression. Further processes for preparing antibodies or antigen-binding antibody fragments are described in WO2007070538 (see page 22 “Antibodies”). The skilled person knows how processes such as phage display libraries (e.g. Morphosys HuCAL Gold) can be compiled and used for discovering antibodies or antigen-binding antibody fragments (see WO2007070538, page 24 ff and Example 1 on page 70, Example 2 on page 72). Further processes for preparing antibodies that use DNA libraries from B-cells are described for example on page 26 (WO2007070538). Processes for humanizing antibodies are described on page 30-32 of WO2007070538 and in detail in Queen, et al., Pros. Natl. Acad. Sci. USA 86:10029-10033, 1989 or in WO09/00786. Furthermore, processes for the recombinant expression of proteins in general and of antibodies in particular are known to the skilled person (see, for example, in Berger and Kimrnel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biolony, (F. M. Ausabel et al. [Eds.], Current Protocols, Green Publishing Associates, Inc./John Wiley & Sons, Inc.); Harlow et al., (Monoclonal Antibodies: A Laboratory Manual, Cold Spring Harbor Laboratory Press (19881, Paul [Ed.]); Fundamental Immunology, (Lippincott Williams & Wilkins (1998)); and Harlow, et al., (Using Antibodies: A Laboratory Manual, Cold Spring Harbor Laboratory Press (1998)). The skilled person knows the corresponding vectors, promoters and signal peptides which are necessary for the expression of a protein/antibody. Commonplace processes are also described in WO2007070538 on pages 41-45. Processes for preparing an IgG1 antibody are described for example in WO2007070538 in Example 6 on page 74 ff. Processes which allow the determination of the internalization of an antibody after binding to its antigen are known to the skilled person and are described for example in WO2007070538 on page 80. The skilled person is able to use the processes described in WO2007070538 that have been used for preparing carboanhydrase IX (Mn) antibodies in analogy for the preparation of antibodies with different target molecule specificity.

Further examples of FGFR2 binders are the single chain Fv antibody fragments PRO-007 (binds FGFR2 with high affinity) and PRO-001 (binds FGFR3 with high affinity and FGFR2 with low affinity) described in WO2007144893.

The compounds of the invention possess valuable pharmacological properties and can be used for the prevention and treatment of diseases in humans and animals.

The binder-drug conjugates (ADCs) of the invention, of the formula (Ia), exhibit a high and specific cytotoxic activity with regard to tumour cells, as may be shown on the basis of the assays set out in the present experimental section (Section C). This high and specific cytotoxic activity on the part of the binder-drug conjugates (ADCs) of the invention, of the formula (Ia), is achieved through the appropriate combination of the new N,N-dialkylauristatin derivative and binder with linkers which exhibit not only an enzymatically, hydrolytically or reductively cleavable predetermined break point, for the release of the toxophores, but also no such predetermined break point. More particularly, through the use of stable linkers which have no enzymatically, hydrolytically or reductively cleavable predetermined break point for the release of the toxophores, and which, following uptake of the ADCs into the tumour cell and following complete intracellular, enzymatic breakdown of the antibody, still remain wholly or partly intact, the activity is confined very specifically to the tumour cell. Compatibility between ADCs and stable linkers presupposes, among other things, that the metabolites formed intracellularly can be formed with sufficient efficacy, are able to reach their target and are able there to develop their anti-proliferative activity on the target with sufficient potency, without being carried out of the tumour cell again beforehand by transporter proteins. Such a compatibility of the ADCs with a stabile linker chemistry and with the target in question offers an enlarged therapeutic window (see, e.g., L. Ducry, Bionconjugate Chem. 2010, 21-5; A. G. Polson, Cancer Res. 2009, 69, 2358).

More particularly, the binder-drug conjugates of the invention, of the formula (Ia), exhibit a high and specific cytotoxic activity with respect to tumour cells which express FGFR2. The activity with respect to tumour cells which do not express FGFR2 is significantly weaker at the same time.

On the basis of this profile of properties, the compounds of the invention are therefore suitable to a particular degree for the treatment of hyperproliferative diseases in humans and in mammals generally. The compounds are able on the one hand to inhibit, block, reduce or lower cell proliferation and cell division, and on the other hand to increase apoptosis.

The hyperproliferative diseases for the treatment of which the compounds of the invention can be employed include in particular the group of cancer and tumour diseases. In the context of the present invention, these are understood as meaning, in particular, the following diseases, but without being limited to them: mammary carcinomas and mammary tumours (ductal and lobular forms, also in situ, triple-negative, HER2-negative), tumours of the respiratory tract (parvicellular and non-parvicellular carcinoma, bronchial carcinoma), cerebral tumours (e.g. of the brain stem and of the hypothalamus, astrocytoma, medulloblastoma, ependymoma and neuro-ectodermal and pineal tumours), tumours of the digestive organs (oesophagus, carcinomas of the oesophagogastric junction (=EGJ), stomach (diffuse and intestinal forms), gall bladder, small intestine, large intestine, rectum), liver tumours (including hepatocellular carcinoma, cholangiocellular carcinoma and mixed hepatocellular and cholangiocellular carcinoma), tumours of the head and neck region (larynx, hypopharynx, nasopharynx, oropharynx, lips and oral cavity), skin tumours (squamous epithelial carcinoma, Kaposi sarcoma, malignant melanoma, Merkel cell skin cancer and non-melanomatous skin cancer), tumours of soft tissue (including soft tissue sarcomas, osteosarcomas, malignant fibrous histiocytomas, lymphosarcomas and rhabdomyosarcomas), tumours of the eyes (including intraocular melanoma and retinoblastoma), tumours of the endocrine and exocrine glands (e.g. thyroid and parathyroid glands, pancreas and salivary gland), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testicles in men). These also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hair cell leukaemia, and also AIDS-correlated lymphomas, Hodgkin's lymphomas, non-Hodgkin's lymphomas, cutaneous T-cell lymphomas, Burkitt's lymphomas and lymphomas in the central nervous system.

### Preferred Hyperproliferative Diseases for Anti-FGFR2 Binder-Drug Conjugates

Hyperproliferative diseases for the treatment of which the compounds of the invention can be preferably employed are FGFR2-expressing tumours, such as, for example, stomach carcinoma (intestinal and diffuse types), signet ring carcinoma, especially of diffuse type, oesophageal cancer, cancer of the oesophagogastric junction (EGJ), breast cancer, cancer of the large intenstine, colorectal carcinoma, rectal carcinoma, prostate cancer, kidney cancer, carcinomas of the head and neck region, pancreatic cancer, liver cancer, cervical carcinoma, ovarian carcinoma, endometrial carcinoma, more particularly of endometrioid type, of papillary serous type, or of clear cell subtype, lung cancer, more particularly non-small-cell lung carcinoma (NSCLC), adenocarcinoma, squamous carcinoma and pancreatic carcinoma.

These well-described diseases in humans can also occur with a comparable aetiology in other mammals and can be treated there with the compounds of the present invention.

In the context of this invention, the term “treatment” or “treat” is used conventionally and means the care, management and support of a patient with the aim of combatting, diminishing, attenuating or relieving a disease or health defect and of improving the living conditions which are adversely affected by this disease, such as in the case of a cancer disease.

The present invention thus further provides the use of the compounds of the invention for the treatment and/or prevention of diseases, in particular the abovementioned diseases.

The present invention furthermore provides the use of the compounds of the invention for the preparation of a medicament for the treatment and/or prevention of diseases, in particular the abovementioned diseases.

The present invention furthermore provides the use of the compounds of the invention in a method for the treatment and/or prevention of diseases, in particular the abovementioned diseases.

The present invention furthermore provides a method for the treatment and/or prevention of diseases, in particular the abovementioned diseases, using an effective amount of at least one of the compounds of the invention.

The compounds according to the invention can be employed by themselves or, if required, in combination with one or more other pharmacologically active substances, as long as this combination does not lead to undesirable and unacceptable side effects. The present invention furthermore therefore provides medicaments comprising at least one of the compounds of the invention and one or more further drugs, in particular for the treatment and/or prevention of the abovementioned diseases.

For example, the compounds of the present invention can be combined with known antihyperproliferative, cytostatic or cytotoxic substances for the treatment of cancer diseases. Suitable drugs in the combination which may be mentioned by way of example are as follows:

aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, Synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, BAY 43-9006 (sorafenib), CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example without this list being conclusive as follows:

aminoglutethimide, L-asparaginase, azathioprine, 5-azacytidine, bleomycin, busulfan, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenin, ethinylestradiol, etoposide, fludarabin phosphate, 5-fluorodeoxyuridine, 5-fluorodeoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds of the invention can also be combined in a very promising manner with biological therapeutics such as antibodies (e.g. avastin, rituxan, erbitux, herceptin). The compounds of the invention can also achieve positive effects in combination with therapies directed against angiogenesis, such as, for example, with avastin, axitinib, recentin, regorafenib, sorafenib or sunitinib. Combinations with inhibitors of the proteasome and of mTOR and also with antihormones and steroidal metabolic enzyme inhibitors are likewise particularly suitable because of their favourable profile of side effects.

Generally, the following aims can be pursued with the combination of compounds of the present invention with other agents having a cytostatic or cytotoxic action:
an improved activity in slowing down the growth of a tumour, in reducing its size or even in its complete elimination compared with treatment with an individual drug;
the possibility of employing the chemotherapeutics used in a lower dosage than in monotherapy;
the possibility of a more tolerable therapy with few side effects compared with individual administration;
the possibility of treatment of a broader spectrum of tumour diseases;
the achievement of a higher rate of response to the therapy;
a longer survival time of the patient compared with present-day standard therapy.

The compounds according to the invention can moreover also be employed in combination with radiotherapy and/or surgical intervention.

The present invention furthermore provides medicaments which comprise at least one compound of the invention, conventionally together with one or more inert, non-toxic, pharmaceutically suitable excipients, and the use thereof for the abovementioned purposes.

The compounds of the invention can act systemically and/or locally. They can be administered in a suitable manner for this purpose, such as for example parenterally, possibly by means of inhalation, or as an implant or stent.

The compounds of the invention can be administered in suitable administration forms for these administration routes.

Parenteral administration can be effected with bypassing of an absorption step (e.g. intravenously, intraarterially, intracardially, intraspinally or intralumbally) or with inclusion of an absorption (e.g. intramuscularly, subcutaneously, intracutaneously, percutaneously or intraperitoneally). Administration forms which are suitable for parenteral administration include injection and infusion formulations in the form of solutions, suspensions, emulsions or lyophilizates. Parenteral administration is preferred, in particular intravenous administration.

In general, it has proved advantageous in the case of parenteral administration to administer amounts of from about 0.001 to 1 mg/kg, preferably about 0.01 to 0.5 mg/kg of body weight to achieve effective results.

Nevertheless it may be necessary to deviate from the amounts mentioned, and in particular depending on the body weight, administration route, individual behaviour towards the active compound, nature of the formulation and point of time or interval at which administration takes place. Thus in some cases it may be sufficient to manage with less than the abovementioned minimum amount, while in other cases the upper limit mentioned must be exceeded. In the case where relatively large amounts are administered, it may be advisable to distribute these into several individual doses over the day.

The following examples illustrate the invention. The invention is not limited to the examples.

The percentage figures in the following tests and examples are percentages by weight, unless stated otherwise; parts are parts by weight. Solvent ratios, dilution ratios and concentration data of liquid/liquid solutions in each case relate to the volume.

### A. EXAMPLES

### Abbreviations and Acronyms

ABCB1 ATP-binding cassette sub-family B member 1 (synonym for P-gp and MDR1)
abs. absolute
ADC antibody-drug-conjugate
Ac acetyl
aq. aqueous, aqueous solution
ATP adenosine triphosphate
BCRP breast cancer resistance protein, an efflux transporter
Boc tert-butoxycarbonyl
br. broad (in NMR)
Ex. example
ca. circa, approximately
CAIX carboanhydrase IX
CI chemical ionization (in MS)
d doublet (in NMR)
d day(s)
TLC thin-layer chromatography
DCI direct chemical ionization (in MS)
dd doublet of a doublet (in NMR)
DMAP 4-N,N-dimethylaminopyridine
DME 1,2-dimethoxyethane
DMEM Dulbecco's modified eagle medium (standardized nutrient medium for cell culture)
DMF N,N-dimethylformamide
DMSO dimethyl sulphoxide
DPBS, D-PBS, PBS Dulbecco's phosphate-buffered saline solution
   PBS=DPBS=D-PBS, pH 7.4, from Sigma, No. D8537
   Composition:
   0.2 g KCl
   0.2 g KH₂PO₄ (anhydrous)
   8.0 g NaCl
   1.15 g Na₂HPO₄ (anhydrous)
   make up to 11 with H₂O
dt doublet of a triplet (in NMR)
DTT DL-dithiothreitol
EDC N′-(3-dimethylaminopropyl)-N-ethylcarbodiimide hydrochloride
EGFR epidermal growth factor receptor
EI electron impact ionization (in MS)
ELISA enzyme-linked immunosorbent assay
eq. equivalent(s)
ESI electrospray ionization (in MS)
ESI-MicroTofq ESI-MicroTofq (name of the mass spectrometer, with Tof=time of flight and q=quadrupole)
FCS foetal calf serum
FGFR2 fibroblast growth factor receptor 2
Fmoc (9H-fluoren-9-ylmethoxy)carbonyl
sat. saturated
GTP guanosine 5′-triphosphate
h hour(s)
HATU O-(7-azabenzotriazol-1-yl)-N,N,N′,N′-tetramethyluronium hexafluorophosphate
HEPES 4-(2-hydroxyethyl)piperazine-1-ethanesulphonic acid
HOAc acetic acid
HOBt 1-hydroxy-1H-benzotriazole hydrate
HOSu N-hydroxysuccinimide
HPLC high-pressure, high-performance liquid chromatography
IC₅₀ half-maximum inhibitory concentration
i.m. intramuscular, administration into the muscle
i.v. intravenous, administration into the vein
Kato III human tumour cell line
conc. concentrated
LC-MS liquid chromatography-coupled mass spectrometry
LLC-PK1 cells Lewis lung carcinoma pork kidney cell line
L-MDR human MDR1 transfected LLC-PK1 cells
m multiplet (in NMR)
MDA-MB-231 human tumour cell line
MDR1 multidrug resistence protein 1
MFM-223 human tumour cell line
min minute(s)
MS mass spectrometry
MTT 3-(4,5-dimethylthiazol-2-yl)-2,5-diphenyl-2H-tetrazolium bromide
NCI-H716 human tumour cell line
NMM N-methylmorpholine
NMP N-methyl-2-pyrrolidinone
NMR nuclear magnetic resonance spectrometry
NMRI mouse strain, originating from Naval Medical Research Institute (NMRI)
Nude mice experimental animals
NSCLC non-small cell lung cancer (non-parvicellular bronchial carcinoma)
PBS phosphate-buffered saline solution
Pd/C palladium on activated carbon
P-gp P-glycoprotein, a transporter protein
PNGaseF enzyme for sugar elimination
quant. quantitative (for yield)
quart quartet (in NMR)
quint quintet (in NMR)
R_{f} retention index (for TLC)
RT room temperature
Rₜ retention time (for HPLC)
s singlet (in NMR)
S.C. subcutaneous, administration beneath the skin
SCID mice experimental mice with a severe combined immunodeficiency
SNU-16 human tumour cell line
SUM52-PE human tumour cell line
t triplet (in NMR)
tert tertiary
TFA trifluoroacetic acid
THF tetrahydrofuran
UV ultraviolet spectrometry
v/v volume to volume ratio (of a solution)
Z benzyloxycarbonyl

### HPLC and LC-MS Methods:

### Method 1 (LC-MS):

Instrument: Waters Acquity SQD UPLC System; column: Waters Acquity UPLC HSS T3 1.8μ 50 mm×1 mm; eluent A: 11 water+0.25 ml 99% strength formic acid, eluent B: 11 acetonitrile+0.25 ml 99% strength formic acid; gradient: 0.0 min 90% A→1.2 min 5% A→2.0 min 5% A; flow rate: 0.40 ml/min; oven: 50° C.; UV detection: 210-400 nm

### Method 2 (LC-MS):

Instrument: Micromass QuattroPremier with Waters UPLC Acquity; column: Thermo Hypersil GOLD 1.9μ 50 mm×1 mm; eluent A: 11 water+0.5 ml 50% strength formic acid, eluent B: 11 acetonitrile+0.5 ml 50% strength formic acid; gradient: 0.0 min 90% A→0.1 min 90% A→1.5 min 10% A→2.2 min 10% A; flow rate: 0.33 ml/min; oven: 50° C.; UV detection: 210 nm

### Method 3 (LC-MS):

Instrument: Micromass Quattro Micro MS with HPLC Agilent Series 1100; column: Thermo Hypersil GOLD 3μ 20 mm×4 mm; eluent A: 11 water+0.5 ml 50% strength formic acid, eluent B: 11 acetonitrile+0.5 ml 50% strength formic acid; gradient: 0.0 min 100% A→3.0 min 10% A→4.0 min 10% A→4.01 min 100% A (flow rate 2.5 ml/min)→5.00 min 100% A; oven: 50° C.; flow rate: 2 ml/min; UV detection: 210 nm

### Method 4 (LC-MS):

MS instrument: Micromass ZQ; HPLC instrument: HP 1100 Series; UV DAD; column: Phenomenex Gemini 3 g 30 mm×3.00 mm; eluent A: 11 water+0.5 ml 50% strength formic acid, eluent B: 11 acetonitrile+0.5 ml 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm

### Method 5 (HPLC):

Instrument: HP 1090 Series II; column: Merck Chromolith SpeedROD RP-18e, 50 mm×4.6 mm; preliminary column: Merck Chromolith Guard Cartridge Kit RP-18e, 5 mm×4.6 mm; injection volume: 5 μl; eluent A: 70% HClO₄ in water (4 ml/litre), eluent B: acetonitrile; gradient: 0.00 min 20% B→0.50 min 20% B→3.00 min 90% B→3.50 min 90% B→3.51 min 20% B→4.00 min 20% B; flow rate: 5 ml/min; column temperature: 40° C.

### Method 6 (HPLC):

Instrument: Waters 2695 with DAD 996; column: Merck Chromolith SpeedROD RP-18e, 50 mm×4.6 mm; Ord. No.: 1.51450.0001, preliminary column: Merck Chromolith Guard Cartridge Kit RP-18e, 5 mm×4.6 mm; Ord. No.: 1.51470.0001, eluent A: 70% HClO₄ in water (4 ml/litre), eluent B: acetonitrile; gradient: 0.00 min 5% B→0.50 min 5% B→3.00 min 95% B→4.00 min 95% B; flow rate: 5 ml/min

### Method 7 (LC-MS):

MS instrument: Waters ZQ; HPLC instrument: Agilent 1100 Series; UV DAD; column: Thermo Hypersil GOLD 3μ 20 mm×4 mm; eluent A: 11 water+0.5 ml 50% strength formic acid, eluent B: 11 acetonitrile+0.5 ml 50% strength formic acid; gradient: 0.0 min 100% A→3.0 min 10% A→4.0 min 10% A→4.1 min 100% A (flow rate 2.5 ml/min); oven: 55° C.; flow rate: 2 ml/min; UV detection: 210 nm

### Method 8 (LC-MS):

MS instrument: Waters ZQ; HPLC instrument: Agilent 1100 Series; UV DAD; column: Thermo Hypersil GOLD 3μ 20 mm×4 mm; eluent A: 11 water+0.5 ml 50% strength formic acid, eluent B: 11 acetonitrile+0.5 ml 50% strength formic acid; gradient: 0.0 min 100% A→2.0 min 60% A→2.3 min 40% A→3.0 min 20% A→4.0 min 10% A→4.2 min 100% A (flow rate 2.5 ml/min); oven: 55° C.; flow rate: 2 ml/min; UV detection: 210 nm

### Method 9 (LC-MS):

Instrument: Waters Acquity SQD UPLC System; column: Waters Acquity UPLC HSS T3 1.8μ 50 mm×1 mm; eluent A: 11 water+0.25 ml 99% strength formic acid, eluent B: 11 acetonitrile+0.25 ml 99% strength formic acid; gradient: 0.0 min 95% A→6.0 min 5% A→7.5 min 5% A; oven: 50° C.; flow rate: 0.35 ml/min; UV detection: 210-400 nm

### Method 10 (HPLC):

Instrument: Agilent 1200 Series; column: Agilent Eclipse XDB-C18 5μ 4.6 mm×150 mm; preliminary column: Phenomenex KrudKatcher Disposable Pre-Column; injection volume: 5 μl; eluent A: 11 water+0.01% trifluoroacetic acid; eluent B: 11 acetonitrile+0.01% trifluoroacetic acid; gradient: 0.00 min 10% B→1.00 min 10% B→1.50 min 90% B→5.5 min 10% B; flow rate: 2 ml/min; column temperature: 30° C.

For all reactants or reagents whose preparation is not explicitly described below, they were obtained commercially from generally available sources. For all other reactants or reacents whose preparation is likewise not described below, and which were not available commercially or were obtained from sources which are not generally available, a reference is given to the published literature in which their preparation is described.

### Method 11 (LC-MS):

Instrument: Waters ACQUITY SQD UPLC System; column: Waters Acquity UPLC HSS T3 1.8μ 30×2 mm; eluent A: 11 water+0.25 ml 99% strength formic acid, eluent B: 11 acetonitrile+0.25 ml 99% strength formic acid; gradient: 0.0 min 90% A→1.2 min 5% A→2.0 min 5% A oven: 50° C.; flow rate: 0.60 ml/min; UV detection: 208-400 nm

### Method 12 (HPLC):

Instrument: Agilent 1200 Series with column oven and DAD; column: Merck Chromolith SpeedROD RP-18e, 50 mm×4.6 mm; Ord. No.: 1.51450.0001; preliminary column: Merck Chromolith Guard Cartridge Kit RP-18e, 5 mm×4.6 mm; Ord. No.: 1.51470.0001; eluent A: 70% HClO₄ in water (4 ml/litre), eluent B: acetonitrile; gradient: 0.00 min 5% B→0.50 min 5% B→3.00 min 95% B→4.00 min 95% B; flow rate: 5 ml/min; column temperature: 30° C.

### Starting Compounds and Intermediates

### Starting Compound 1

### 2R,3R)-3-[(2S)-1-(tert-butoxycarbonyl)pyrrolidin-2-yl]-3-methoxy-2-methylpropanoic acid (Boc-dolaproine)

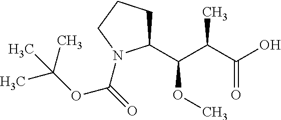 [SMILES: CO[C@H]([C@@H](C)C(=O)O)[C@@H]1CCCN1C(=O)OC(C)(C)C]

The title compound can be prepared in various ways according to literature methods; see, for example, Pettit et al., Synthesis 1996, 719; Shioiri et al., Tetrahedron Lett. 1991, 32, 931; Shioiri et al., Tetrahedron 1993, 49, 1913; Koga et al., Tetrahedron Lett. 1991, 32, 2395; Vidal et al., Tetrahedron 2004, 60, 9715; Poncet et al., Tetrahedron 1994, 50, 5345. It was prepared either as the free acid or as a 1:1 salt with dicyclohexylamine

### Starting Compound 2a

### tert-butyl (3R,4S,5S)-3-methoxy-5-methyl-4-(methylamino)heptanoate hydrochloride (dolaisoleucine-OtBu×HCl)

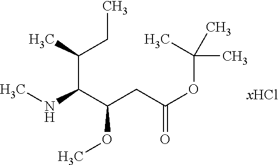 [SMILES: CC[C@H](C)[C@H](NC)[C@@H](CC(=O)OC(C)(C)C)OC]

The title compound can be prepared in various ways according to literature methods; see, for example, Pettit et al., J. Org. Chem. 1994, 59, 1796; Koga et al., Tetrahedron Lett. 1991, 32, 2395; Shioiri et al., Tetrahedron Lett. 1991, 32, 931; Shioiri et al., Tetrahedron 1993, 49, 1913.

### Starting Compound 2b

### tert-butyl (3R,4S,5S)-3-methoxy-5-methyl-4-(methylamino)heptanoate (dolaisoleucine-O^{t}Bu)

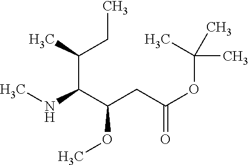 [SMILES: CC[C@H](C)[C@H](NC)[C@@H](CC(=O)OC(C)(C)C)OC]

The compound was prepared in analogy to starting compound 2a, except that the hydrogenation was performed without addition of 1N hydrochloric acid.

### Starting Compound 3

### Nα-(tert-butoxycarbonyl)-N-hydroxy-L-phenylalaninamide

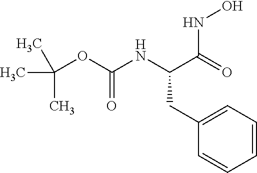 [SMILES: CC(C)(C)OC(=O)N[C@@H](Cc1ccccc1)C(=O)NO]

The title compound was prepared by the literature method (A. Ritter et al., J. Org. Chem. 1994, 59, 4602).

Yield: 750 mg (75% of theory)

LC-MS (Method 3): Rₜ=1.67 min; MS (ESIpos): m/z=281 (M+H)⁺.

### Starting Compound 4

### 1,2-oxazolidine hydrochloride

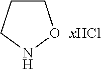

The title compound can be prepared by literature methods (see, for example, H. King, J. Chem. Soc. 1942, 432); it is also commercially available.

### Starting Compound 5

### 1,2-oxazinane hydrochloride

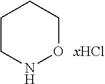

The title compound can be prepared by literature methods (see, for example, H. King, J. Chem. Soc. 1942, 432).

### Starting Compound 6

### 2-oxa-3-azabicyclo[2.2.2]oct-5-ene

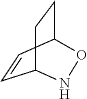

The title compound can be prepared in Boc-protected form by the literature method (see, for example, C. Johnson et al., Tetrahedron Lett. 1998, 39, 2059); the deprotection was effected in a customary manner by treatment with trifluoroacetic acid and subsequent neutralization.

Yield: 149 mg (89% of theory)

### Starting Compound 7

### tert-butyl (1S,2R)-1-(hydroxycarbamoyl)-2-phenylcyclopropyl carbamate

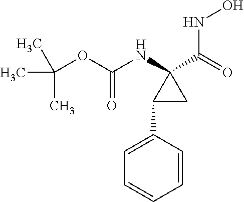 [SMILES: CC(C)(C)OC(=O)N[C@@]1(C(=O)NO)C[C@@H]1c1ccccc1]

The title compound was prepared by a literature method (A. Ritter et al., J. Org. Chem. 1994, 59, 4602) proceeding from commercially available (1S,2R)-1-[(tert-butoxycarbonyl)amino]-2-phenylcyclopropanecarboxylic acid (C. Cativiela et al., Chirality 1999, 11, 583).

Yield: 339 mg (59% of theory)

LC-MS (Method 1): Rₜ=0.82 min; MS (ESIpos): m/z=293 (M+H)⁺.

### Intermediate 1

### tert-butyl (3R,4S,5S)-4-[{N-[(benzyloxy)carbonyl]-L-valyl}(methyl)amino]-3-methoxy-5-methylheptanoate

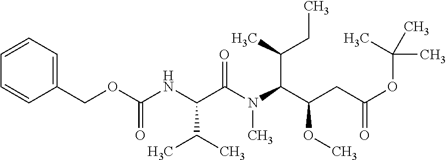 [SMILES: CC[C@H](C)[C@@H]([C@@H](CC(=O)OC(C)(C)C)OC)N(C)C(=O)[C@@H](NC(=O)OCc1ccccc1)C(C)C]

10.65 g (41.058 mmol) of tert-butyl (3R,4S,5S)-3-methoxy-5-methyl-4-(methylamino)heptanoate (starting compound 2b) were taken up in 250 ml of dichloromethane and the solution was cooled to −10° C. Then, while stirring, 10.317 g (41.058 mmol) of N-[(benzyloxy)carbonyl]-L-valine, 16.866 g (61.586 mmol) of 2-bromo-1-ethylpyridinium tetrafluoroborate (BEP) and 28.6 ml of N,N-diisopropylethylamine were added, and the mixture was subsequently stirred at RT for 20 h. The reaction mixture was then diluted with dichloromethane and shaken twice with saturated sodium chloride solution, dried over sodium sulphate, filtered and concentrated. The residue was purified by flash chromatography on silica gel with 4:1 petroleum ether/ethyl acetate as the eluent. The corresponding fractions were concentrated and the residue was dried under high vacuum overnight. 10.22 g (51% of theory) of the title compound were obtained as a yellowish oil.

HPLC (Method 5): Rₜ=2 3 min;

LC-MS (Method 2): Rₜ=1.59 min; MS (ESIpos): m/z=493 (M+H)⁺.

### Intermediate 2

### tert-butyl (3R,4S,5S)-3-methoxy-5-methyl-4-[methyl(L-valyl)amino]heptanoate

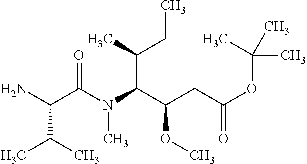 [SMILES: CC[C@H](C)[C@@H]([C@@H](CC(=O)OC(C)(C)C)OC)N(C)C(=O)[C@@H](N)C(C)C]

500 mg (1 mmol) of tert-butyl (3R,4S,5S)-4-[{N-[(benzyloxy)carbonyl]-L-valyl}(methyl)amino]-3-methoxy-5-methylheptanoate (intermediate 1) were dissolved in 50 ml of methanol and, after addition of 100 mg of 10% palladium on activated carbon, hydrogenated under standard hydrogen pressure at RT for 1 h. The catalyst was then filtered off and the solvent was removed under reduced pressure. This gave 370 mg (quant.) of the title compound as a virtually colourless oil.

HPLC (Method 5): Rₜ=1.59 min;

LC-MS (Method 1): Rₜ=0.74 min; MS (ESIpos): m/z=359 (M+H)⁺.

### Intermediate 3

### N-[(9H-fluoren-9-ylmethoxy)carbonyl]-N-methyl-L-valyl-N-[(3R,4S,5S)-1-tert-butoxy-3-methoxy-5-methyl-1-oxoheptan-4-yl]-N-methyl-L-valinamide

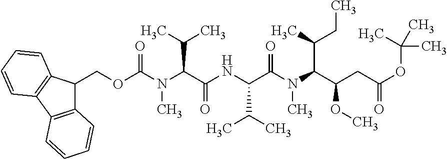 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

4.64 g (13.13 mmol) of N-[(9H-fluoren-9-ylmethoxy)carbonyl]-N-methyl-L-valine were dissolved in 20 ml of DMF and admixed successively with 4.28 g (11.94 mmol) of tert-butyl (3R,4S,5S)-3-methoxy-5-methyl-4-[methyl(L-valyl)amino]heptanoate (Intermediate 2), 2.75 g (14.33 mmol) of 1-(3-dimethylaminopropyl)-3-ethylcarbodiimide hydrochloride and 2.2 g (14.33 mmol) of 1-hydroxy-1H-benzotriazole hydrate. The mixture was stirred at RT overnight. The reaction mixture was then poured into a mixture of semisaturated aqueous ammonium chloride solution and ethyl acetate. The organic phase was removed, washed successively with saturated sodium hydrogencarbonate solution and saturated sodium chloride solution, dried over magnesium sulphate, filtered and concentrated. The residue was used directly in the next stage, without further purification.

Yield: 9.1 g (quant., 60% purity)

HPLC (Method 5): Rₜ=2 7 min;

LC-MS (Method 2): Rₜ=1.99 min; MS (ESIpos): m/z=694 (M+H)⁺.

### Intermediate 4

### N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide

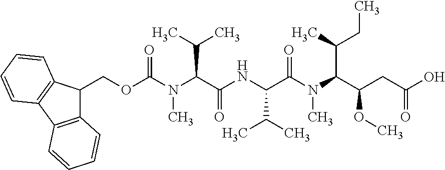 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

9.1 g of the crude product N-[(9H-fluoren-9-ylmethoxy)carbonyl]-N-methyl-L-valyl-N-[(3R,4S,5S)-1-tert-butoxy-3-methoxy-5-methyl-1-oxoheptan-4-yl]-N-methyl-L-valinamide (Intermediate 3) were taken up in 56.6 ml of dichloromethane, 56.6 ml of trifluoroacetic acid were added, and the mixture was stirred at RT for 2 h. Subsequently, the reaction mixture was concentrated under reduced pressure and the remaining residue was purified by flash chromatography, using dichloromethane, 3:1 dichloromethane/ethyl acetate and 15:5:0.5 dichloromethane/ethyl acetate/methanol as eluent. After purification of the corresponding fractions and concentration, 5.8 g (86% of theory) of the title compound were obtained as a colourless foam.

HPLC (Method 5): Rₜ=2.2 min;

LC-MS (Method 1): Rₜ=1.3 min; MS (ESIpos): m/z=638 (M+H)⁺.

### Intermediate 5

### tert-butyl (2S)-1-(1,2-oxazinan-2-yl)-1-oxo-3-phenylpropan-2-yl carbamate

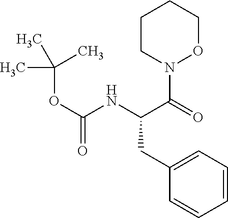 [SMILES: CC(C)(C)OC(=O)N[C@@H](Cc1ccccc1)C(=O)N1CCCCO1]

500 mg (1.9 mmol) of N-(tert-butoxycarbonyl)-L-phenylalanine were dissolved in 10 ml of DMF and admixed successively with 466 mg (3.8 mmol) of 1,2-oxazinane hydrochloride (Starting Compound 5), 433 mg (2.3 mmol) of 1-(3-dimethylaminopropyl)-3-ethylcarbodiimide hydrochloride, 382 mg (2.8 mmol) of 1-hydroxy-1H-benzotriazole hydrate and 731 mg (5.7 mmol) of N,N-diisopropylethylamine. The mixture was stirred at RT overnight. The reaction mixture was then poured into a mixture of semisaturated aqueous ammonium chloride solution and ethyl acetate. The organic phase was removed, washed successively with saturated sodium hydrogencarbonate solution and saturated sodium chloride solution, dried over magnesium sulphate, filtered and concentrated. 620 mg (98% of theory) of the title compound were obtained.

HPLC (Method 5): Rₜ=1.8 min;

LC-MS (Method 2): Rₜ=1.62 min; MS (ESIpos): m/z=235 (M-C₄H₈—CO₂+H)⁺.

### Intermediate 6

### (2S)-2-amino-1-(1,2-oxazinan-2-yl)-3-phenylpropan-1-one trifluoroacetate

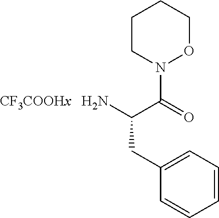 [SMILES: N[C@@H](Cc1ccccc1)C(=O)N1CCCCO1]

620 mg (1.85 mmol) of tert-butyl (2S)-1-(1,2-oxazinan-2-yl)-1-oxo-3-phenylpropan-2-yl carbamate (Intermediate 5) were taken up in 5 ml of dichloromethane, 10 ml of trifluoroacetic acid were added and the mixture was stirred at RT for 30 min Subsequently, the reaction mixture was concentrated under reduced pressure and the remaining residue was lyophilized from water/acetonitrile. In this way, 779 mg (91% of theory) of the title compound were obtained as a colourless foam.

HPLC (Method 5): Rₜ=0.45 min;

LC-MS (Method 3): Rₜ=1.09 min; MS (ESIpos): m/z=235 (M+H)⁺.

### Intermediate 7

### (2R,3R)-3-methoxy-2-methyl-N-[(2S)-1-(1,2-oxazinan-2-yl)-1-oxo-3-phenylpropan-2-yl]-3-[(2S)-pyrrolidin-2-yl]propanamide trifluoroacetate

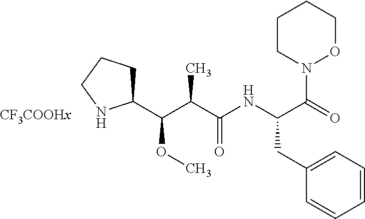 [SMILES: CO[C@@H]([C@@H]1CCCN1)[C@@H](C)C(=O)N[C@@H](Cc1ccccc1)C(=O)N1CCCCO1]

360 mg (1.25 mmol) of (2R,3R)-3-[(2S)-1-(tert-butoxycarbonyl)pyrrolidin-2-yl]-3-methoxy-2-methylpropanoic acid (Starting Compound 1) were taken up in 10 ml of DMF and admixed successively with 579.2 mg (1.25 mmol) of (2S)-2-amino-1-(1,2-oxazinan-2-yl)-3-phenylpropan-1-one trifluoroacetate (Intermediate 6), 714.5 mg (1.88 mmol) of O-(7-azabenzotriazol-1-yl)-N,N,N′,N′-tetramethyluronium hexafluorophosphate (HATU) and 655 μl of N,N-diisopropylethylamine. The mixture was stirred at RT for 16 h. The reaction mixture was then concentrated, and the residue was taken up in ethyl acetate and extracted by shaking first with 5% aqueous citric acid solution, then with 5% aqueous sodium hydrogencarbonate solution and subsequently with saturated sodium chloride solution. The organic phase was concentrated and the residue was purified by flash chromatography on silica gel with 16:4 dichloromethane/methanol as the eluent. The corresponding fractions were combined and the solvent was removed under reduced pressure. After the residue had been dried under high vacuum, 503.5 mg (74% of theory) of the Boc-protected intermediate tert-butyl (2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidine-1-carboxylate were obtained.

HPLC (Method 12): Rₜ=2.0 min;

LC-MS (Method 1): Rₜ=1.12 min; MS (ESIpos): m/z=504 (M+H)+.

503 mg (1 mmol) of this intermediate were taken up in 20 ml of dichloromethane, 10 ml of trifluoroacetic acid were added, and the mixture was stirred at RT for 30 min Subsequently, the reaction mixture was concentrated under reduced pressure and redistilled with dichloromethane. The remaining residue was precipitated from ethyl acetate with n-pentane, and the solvent was decanted off. The residue thus obtained was dissolved in water and extracted by shaking with ethyl acetate, and the aqueous phase was subsequently lyophilized. In this way, 462 mg (89% of theory) of the title compound were obtained as a colourless foam.

HPLC (Method 12): Rₜ=1.53 min;

LC-MS (Method 11): Rₜ=0.57 min; MS (ESIpos): m/z=404 (M+H)⁺.

### Intermediate 8

### N-(tert-butoxycarbonyl)-N-methyl-L-valyl-N-[(2R,3S,4S)-1-carboxy-2-methoxy-4-methylhexan-3-yl]-N-methyl-L-valinamide

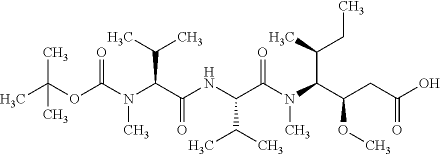 [SMILES: CC[C@H](C)[C@@H]([C@@H](CC(=O)O)OC)N(C)C(=O)[C@@H](NC(=O)[C@H](C(C)C)N(C)C(=O)OC(C)(C)C)C(C)C]

51 mg (0.08 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were dissolved in 10 ml of DMF, and 0.5 ml of piperidine was added. After stirring at RT for 10 min, the reaction mixture was concentrated under reduced pressure and the residue was stirred with diethyl ether. The insoluble constituents were filtered off and washed repeatedly with diethyl ether. Then the filter residue was taken up in 5 ml of dioxane/water and the solution was adjusted to pH 11 with 1 N sodium hydroxide solution. Under ultrasound treatment, a total of 349 mg (1.6 mmol) of di-tert-butyl dicarbonate were added in several portions, in the course of which the pH of the solution was kept at 11. After the reaction had ended, the dioxane was evaporated off and the aqueous solution was adjusted to a pH of 2-3 with citric acid. The mixture was extracted twice with 50 ml each time of ethyl acetate. The organic phases were combined, dried over magnesium sulphate and concentrated under reduced pressure. The residue was taken up in diethyl ether and the of the title compound was precipitated with pentane. The solvent was removed by decantation. The residue was digested several times more with pentane and finally dried under high vacuum. 40 mg (97% of theory) of the title compound were thus obtained.

HPLC (Method 6): Rₜ=2.2 min;

LC-MS (Method 2): Rₜ=1.32 min; MS (ESIpos): m/z=516 (M+H)⁺.

### Intermediate 9

### tert-butyl (2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidine-1-carboxylate

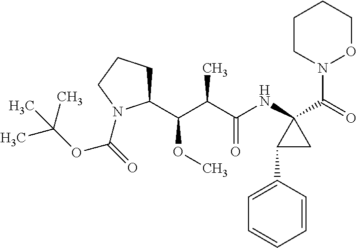 [SMILES: CO[C@H]([C@@H](C)C(=O)N[C@@]1(C(=O)N2CCCCO2)C[C@@H]1c1ccccc1)[C@@H]1CCCN1C(=O)OC(C)(C)C]

The title compound was prepared in analogy to the synthesis of Intermediates 5, 6 and 7 over three stages, by coupling of commercially available (1S,2R)-1-[(tert-butoxycarbonyl)amino]-2-phenylcyclopropanecarboxylic acid with 1,2-oxazinane hydrochloride (Starting Compound 5), subsequent deprotection with trifluoroacetic acid and coupling with Starting Compound 1. The end product was purified by preparative HPLC.

HPLC (Method 5): Rₜ=2.12 min;

LC-MS (Method 2): Rₜ=1.25 min; MS (ESIpos): m/z=516 (M+H)⁺.

### Intermediate 10

### N-[(9H-fluoren-9-ylmethoxy)carbonyl]-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide

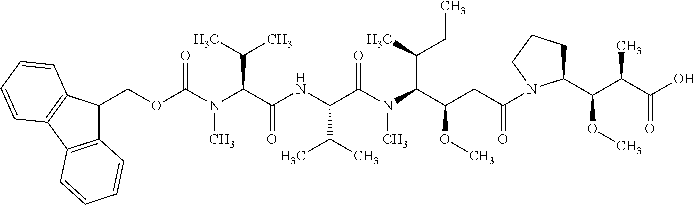 [SMILES: CC[C@H](C)[C@@H]([C@@H](CC(=O)N1CCC[C@H]1[C@H](OC)[C@@H](C)C(=O)O)OC)N(C)C(=O)[C@@H](NC(=O)[C@H](C(C)C)N(C)C(=O)OCC1c2ccccc2-c2ccccc21)C(C)C]

315 mg (0.494 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were dissolved in 12 ml of DMF, and admixed with 104 mg (0.543 mmol) of 1-(3-dimethylaminopropyl)-3-ethylcarbodiimide hydrochloride and 83 mg (0.543 mmol) of 1-hydroxy-1H-benzotriazole hydrate, and the mixture was stirred at RT for 90 min Subsequently, 112 μl of N,N-diisopropylethylamine and 149 mg (0.494 mmol) of (2R,3R)-3-methoxy-2-methyl-3-[(2S)-pyrrolidin-2-yl]propanoic acid trifluoroacetate, which had been prepared beforehand from Starting Compound 1 by elimination of the Boc protecting group by means of trifluoroacetic acid, were added. The mixture was stirred at RT for 2 h and then concentrated under high vacuum. The remaining residue was purified twice by preparative HPLC. 140 mg (35% of theory) of the title compound were obtained in the form of a colourless foam.

HPLC (Method 5): Rₜ=2.40 min;

LC-MS (Method 1): Rₜ=1.38 min; MS (ESIpos): m/z=807 (M+H)⁺.

### Intermediate 11

### N-[(benzyloxy)carbonyl]-N-methyl-L-threonyl-N-[(2R,3S,4S)-1-carboxy-2-methoxy-4-methylhexan-3-yl]-N-methyl-L-valinamide

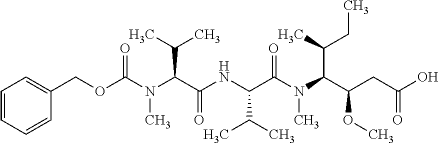 [SMILES: CC[C@H](C)[C@@H]([C@@H](CC(=O)O)OC)N(C)C(=O)[C@@H](NC(=O)[C@H](C(C)C)N(C)C(=O)OCc1ccccc1)C(C)C]

First, N-[(benzyloxy)carbonyl]-N-methyl-L-threonine was released from 237 mg (0.887 mmol) of its dicyclohexylamine salt thereof by taking it up in ethyl acetate and extractive shaking with 5% aqueous sulphuric acid. The organic phase was dried over magnesium sulphate, filtered and concentrated. The residue was taken up in 16 ml of DMF and admixed successively with 365 mg (1 mmol) of tert-butyl (3R,4S,5S)-3-methoxy-5-methyl-4-[methyl(L-valyl)amino]heptanoate (Intermediate 2), 185 mg (0.967 mmol) of 1-(3-dimethylaminopropyl)-3-ethylcarbodiimide hydrochloride and 148 mg (0.967 mmol) of 1-hydroxy-1H-benzotriazole hydrate. The mixture was stirred at RT for 2 h. The reaction mixture was then poured into a mixture of semisaturated aqueous ammonium chloride solution and ethyl acetate. The organic phase was removed, washed successively with saturated sodium hydrogencarbonate solution and saturated sodium chloride solution, dried over magnesium sulphate, filtered and concentrated. The residue was purified by preparative HPLC. 283 mg (53% of theory) of the tert-butyl ester intermediate N-[(benzyloxy)carbonyl]-N-methyl-L-threonyl-N-[(3R,4S,5S)-1-tert-butoxy-3-methoxy-5-methyl-1-oxoheptan-4-yl]-N-methyl-L-valinamide were thus obtained.

HPLC (Method 5): Rₜ=2.17 min 283 mg (0.466 mmol) of this intermediate were taken up in 5 ml of dichloromethane, 5 ml of anhydrous trifluoroacetic acid were added, and the mixture was stirred at RT for 2 h. Subsequently, the reaction mixture was concentrated under high vacuum and the remaining residue was purified by means of preparative HPLC. This gave 156 mg (61% of theory) of the title compound as a colourless foam.

HPLC (Method 5): Rₜ=1.50 min;

LC-MS (Method 2): Rₜ=1.09 min; MS (ESIpos): m/z=552 (M+H)⁺.

### Intermediate 12

### benzyl N-{(2R,3R)-3-methoxy-2-methyl-3-[(2S)-pyrrolidin-2-yl]propanoyl}-L-phenylalaninate trifluoroacetate

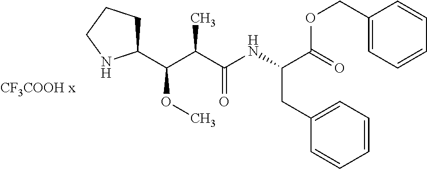 [SMILES: CO[C@@H]([C@@H]1CCCN1)[C@@H](C)C(=O)N[C@@H](Cc1ccccc1)C(=O)OCc1ccccc1]

In the first step, Starting Compound 1 was released from 600 mg (1.28 mmol) of the corresponding dicyclohexylammonium salt by dissolving the salt in 100 ml of ethyl acetate and extractive shaking, first with 50 ml of 0.5% sulphuric acid and then with saturated sodium chloride solution. Then the organic phase was dried over magnesium sulphate, filtered, concentrated and reacted immediately with benzyl L-phenylalaninate in analogy to the synthesis of Intermediate 7, and then deprotected.

Yield: 650 mg (94% over 2 stages)

HPLC (Method 6): Rₜ=1.76 min;

LC-MS (Method 2): Rₜ=1.68 min; MS (ESIpos): m/z=425 (M+H)⁺.

### Intermediate 13

### benzyl (βS)—N-{(2R,3R)-3-methoxy-2-methyl-3-[(2S)-pyrrolidin-2-yl]propanoyl}-β-methyl-L-phenylalaninate trifluoroacetate

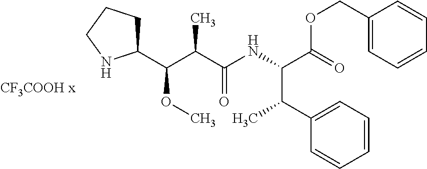 [SMILES: CO[C@@H]([C@@H]1CCCN1)[C@@H](C)C(=O)N[C@H](C(=O)OCc1ccccc1)[C@@H](C)c1ccccc1]

First, (2R,3R)-3-[(2S)-1-(tert-butoxycarbonyl)pyrrolidin-2-yl]-3-methoxy-2-methylpropanoic acid was released from 351 mg (0.75 mmol) of the dicyclohexylamine salt (Starting Compound 1) by taking it up in ethyl acetate and extractive shaking with aqueous 5% potassium hydrogensulphate solution. The organic phase was dried over magnesium sulphate, filtered and concentrated. The residue was taken up in 10 ml of DMF and admixed successively with 373 mg (0.75 mmol) of benzyl ((3S)-β-methyl-L-phenylalaninate trifluoroacetate [prepared from commercially available (βS)—N-(tert-butoxycarbonyl)-β-methyl-L-phenylalanine by EDC/DMAP-mediated esterification with benzyl alcohol and subsequent detachment of the Boc protecting group with trifluoroacetic acid], 428 mg (1.125 mmol) of O-(7-azabenzotriazol-1-yl)-N,N,N′,N′-tetramethyluronium hexafluorophosphate (HATU) and 392 μl of N,N-diisopropylethylamine. The mixture was stirred at RT for 20 h. The reaction mixture was then poured onto a mixture of semisaturated aqueous ammonium chloride solution and ethyl acetate. The organic phase was removed, washed successively with saturated sodium hydrogencarbonate solution and saturated sodium chloride solution, and subsequently concentrated. The residue was purified by means of preparative HPLC. This gave 230 mg (57% of theory) of the Boc-protected intermediate benzyl (βS)—N-{(2R,3R)-3-[(2S)-1-(tert-butoxycarbonyl)pyrrolidin-2-yl]-3-methoxy-2-methylpropanoyl}-β-methyl-L-phenylalaninate.

HPLC (Method 6): Rₜ=2 3 min;

LC-MS (Method 1): Rₜ=1.36 min; MS (ESIpos): m/z=539 (M+H)⁺.

230 mg (0.42 mmol) of this intermediate were taken up in 5 ml of dichloromethane, 5 ml of trifluoroacetic acid were added, and the mixture was stirred at RT for 30 min Subsequently, the reaction mixture was concentrated under reduced pressure. The remaining residue was the reaction mixture dried further under reduced pressure and then lyophilized from acetonitrile/water. In this way, 230 mg (quant.) of the title compound were obtained.

HPLC (Method 6): Rₜ=1.6 min

### Intermediate 14

### N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide trifluoroacetate

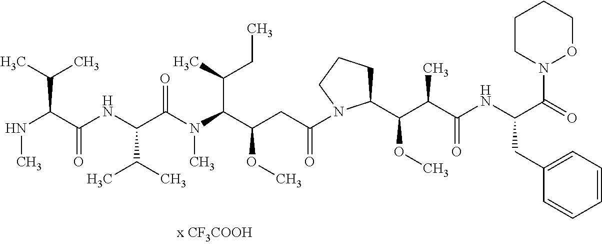 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@@H](NC)C(C)C)C(C)C]

143 mg (0.223 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were taken up in 15 ml of DMF and admixed successively with 141 mg (0.22 mmol) of (2R,3R)-3-methoxy-2-methyl-N-[(2S)-1-(1,2-oxazinan-2-yl)-1-oxo-3-phenylpropan-2-yl]-3-[(2S)-pyrrolidin-2-yl]propanamide trifluoroacetate (Intermediate 7), 102 mg (0.27 mmol) of O-(7-azabenzotriazol-1-yl)-N,N,N′,N′-tetramethyluronium hexafluorophosphate (HATU) and 128 μl (0.74 mmol) of N,N-diisopropylethylamine. The mixture was stirred at RT for 3 h. The reaction mixture was then poured into a mixture of semisaturated aqueous ammonium chloride solution and ethyl acetate. The organic phase was removed, washed successively with saturated sodium hydrogencarbonate solution and saturated sodium chloride solution, dried over magnesium sulphate, filtered and concentrated. This gave 275 mg (quant.) of the Fmoc-protected intermediate N-[(9H-fluoren-9-ylmethoxy)carbonyl]-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide.

HPLC (Method 5): Rₜ=2.73 min;

LC-MS (Method 4): Rₜ=3.19 min; MS (ESIpos): m/z=1023 (M+H)⁺.

46 mg (0.045 mmol) of this intermediate were dissolved in 4 ml of DMF. After 1 ml of piperidine had been added, the reaction mixture was stirred at RT for 1 h. Subsequently, the reaction mixture was concentrated under reduced pressure and the residue was purified by means of preparative HPLC (eluent: acetonitrile+0.01% TFA/water+0.01% TFA). 22 mg (54% of theory) of the title compound were obtained as a colourless foam.

HPLC (Method 5): Rₜ=1.68 min;

LC-MS (Method 2): Rₜ=1.03 min; MS (ESIpos): m/z=801 (M+H)⁺

¹H NMR (600 MHz, DMSO-d₆): δ=8.8 (m, 2H), 8.7 (m, 1H), 8.42 and 8.15 (2d, 1H), 7.3-7.1 (m, 5H), 5.12 and 4.95 (2m, 1H), 4.70 and 4.62 (2m, 1H), 4.62 and 4.50 (2t, 1H), 4.1-3.9 (m, 3H), 3.85 (m, 1H), 3.75-3.6 (m, 2H), 3.23, 3.18, 3.17, 3.14, 3.02 and 2.96 (6s, 9H), 3.1-2.9 and 2.75 (2m, 2H), 2.46 (m, 3H), 2.4-2.1 (m, 2H), 2.05 (br. m, 2H), 1.85-1.55 (br. m, 6H), 1.5-1.2 (br. m, 3H), 1.1-0.8 (m, 18H), 0.75 (t, 3H) [further signals hidden under H₂O peak].

### Intermediate 15

### N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide trifluoroacetate

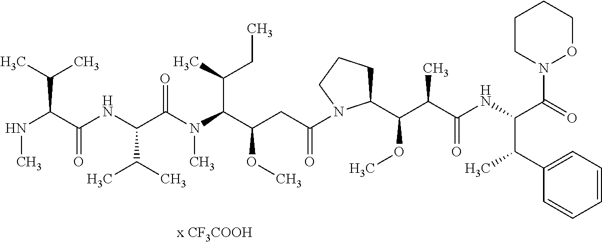 [SMILES: CC[C@H](C)[C@@H]([C@@H](CC(=O)N1CCC[C@H]1[C@H](OC)[C@@H](C)C(=O)N[C@H](C(=O)N1CCCCO1)[C@@H](C)c1ccccc1)OC)N(C)C(=O)[C@@H](NC(=O)[C@@H](NC)C(C)C)C(C)C]

126 mg (0.198 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were taken up in 10 ml of DMF and admixed successively with 105 mg (0.198 mmol) of (2R,3R)-3-methoxy-2-methyl-N-[(2S,3S)-1-(1,2-oxazinan-2-yl)-1-oxo-3-phenylbutan-2-yl]-3-[(2S)-pyrrolidin-2-yl]propanamide trifluoroacetate (Intermediate 17), 41.6 mg (0.217 mmol) of 1-(3-dimethylaminopropyl)-3-ethylcarbodiimide hydrochloride, 33 mg (0.217 mmol) of 1-hydroxy-1H-benzotriazole hydrate and 79 μl (0.454 mmol) of N,N-diisopropylethylamine. The mixture was stirred at RT overnight. The reaction mixture was then poured into a mixture of semisaturated aqueous ammonium chloride solution and ethyl acetate. The organic phase was removed, washed successively with saturated sodium hydrogencarbonate solution and saturated sodium chloride solution, dried over magnesium sulphate, filtered and concentrated. This gave 220 mg (quant.) of the Fmoc-protected intermediate N-[(9H-fluoren-9-ylmethoxy)carbonyl]-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide.

HPLC (Method 5): Rₜ=2.77 min;

LC-MS (Method 1): Rₜ=1.5 min; MS (ESIpos): m/z=1037 (M+H)⁺.

220 mg (0.212 mmol) of this intermediate were dissolved in 5 ml of DMF. After 1 ml of piperidine had been added, the reaction mixture was stirred at RT for 1 h. Subsequently, the reaction mixture was concentrated under reduced pressure and the residue was purified by means of preparative HPLC (eluent: acetonitrile+0.01% TFA water+0.01% TFA). 91 mg (46% of theory) of the title compound were obtained as a colourless foam.

HPLC (Method 5): Rₜ=1.71 min;

LC-MS (Method 1): Rₜ=0.9 min; MS (ESIpos): m/z=815 (M+H)⁺

¹H NMR (600 MHz, DMSO-d₆): δ=8.87 and 8.80 (2d, 2H), 8.75 (m, 1H), 8.40 and 7.98 (2d, 1H), 7.3-7.1 (m, 5H), 5.45 and 5.2 (2t, 1H), 4.78 and 4.62 (2m, 1H), 4.73 and 4.58 (2t, 1H), 4.2-4.0 (m, 3H), 3.7-3.6 (m, 1H), 3.35, 3.20, 3.18, 3.14, 3.12 and 3.00 (6s, 9H), 3.1 and 2.95 (2m, 2H), 2.46 (m, 3H), 2.4-2.0 (m, 4H), 1.9-1.6 (m, 4H), 1.6-1.2 (m, 5H), 1.1-0.75 (m, 21H), 0.80 (t, 3H) [further signals hidden under H₂O peak].

### Intermediate 16

### N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide trifluoroacetate

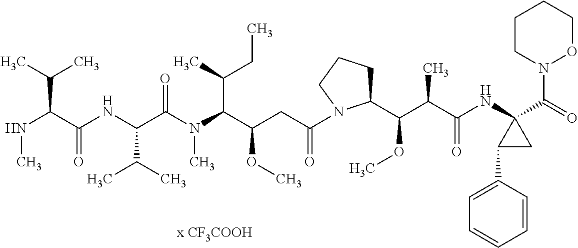 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@@H](NC)C(C)C)C(C)C]

617 mg (1.2 mmol) of tert-butyl (2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidine-1-carboxylate (Intermediate 24) were taken up in 44 ml of dichloromethane, 4.4 ml of trifluoroacetic acid were added and the mixture was stirred at RT for 30 min Subsequently, the reaction mixture was concentrated under reduced pressure and the remaining residue was lyophilized from dioxane/water. 702 mg (quant.) of the deprotected compound (2R,3R)-3-methoxy-2-methyl-N-[(1S,2R)-1-(1,2-oxazinan-2-ylcarbonyl)-2-phenylcyclopropyl]-3-[(2S)-pyrrolidin-2-yl]propanamide trifluoroacetate were obtained as a crude product, which was used in the following stage without further purification.

470 mg (0.74 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were taken up in 57 ml of DMF and admixed successively with 390 mg (approx. 0.74 mmol) of the above-obtained (2R,3R)-3-methoxy-2-methyl-N-[(1S,2R)-1-(1,2-oxazinan-2-ylcarbonyl)-2-phenylcyclopropyl]-3-[(2S)-pyrrolidin-2-yl]propanamide trifluoroacetate, 336 mg (0.88 mmol) of O-(7-azabenzotriazol-1-yl)-N,N,N′,N′-tetramethyluronium hexafluorophosphate (HATU) and 423 μl (2.4 mmol) of N,N-diisopropylethylamine. The mixture was stirred at RT for 2 h. The reaction mixture was then poured into a mixture of semisaturated aqueous ammonium chloride solution and ethyl acetate. The organic phase was removed, washed successively with saturated sodium hydrogencarbonate solution and saturated sodium chloride solution, dried over sodium sulphate, filtered and concentrated. The residue was purified by preparative HPLC. This gave 453 mg (59% of theory) of the Fmoc-protected intermediate N-[(9H-fluoren-9-ylmethoxy)carbonyl]-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

HPLC (Method 5): Rₜ=2.58 min;

LC-MS (Method 1): Rₜ=3.10 min; MS (ESIpos): m/z=1035 (M+H)⁺.

453 mg (0.438 mmol) of this intermediate were dissolved in 24 ml of DMF. After 2.4 ml of piperidine had been added, the reaction mixture was stirred at RT for 30 min Subsequently, the reaction mixture was concentrated under reduced pressure and the residue was purified by preparative HPLC (eluent: acetonitrile/0.1% TFA in water). 260 mg (64% of theory) of the title compound were obtained as a colourless foam.

HPLC (Method 5): Rₜ=1.64 min;

LC-MS (Method 1): Rₜ=0.86 min; MS (ESIpos): m/z=813 (M+H)⁺

¹H NMR (400 MHz, DMSO-d₆): δ=8.8 (m, 2H), 8.65 (m, 2H), 7.3-7.1 (m, 5H), 4.8-4.05 (m, 2H), 4.0 and 3.82 (2m, 2H), 3.8-3.5 (m, 8H), 3.32, 3.29, 3.20, 3.19, 3.12 and 3.00 (6s, 9H), 2.65 (t, 1H), 2.5-2.45 (m, 3H), 2.4-1.3 (m, 15H), 1.15-0.85 (m, 18H), 0.8 and 0.75 (2d, 3H) [further signals hidden under H₂O peak].

### Intermediate 17

### N-benzyl-N-methyl-L-phenylalaninamide trifluoroacetate

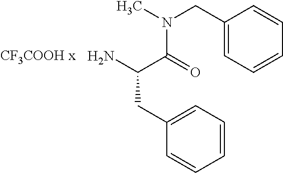 [SMILES: CN(Cc1ccccc1)C(=O)[C@@H](N)Cc1ccccc1]

1000 mg (3.77 mmol) of N-(tert-butoxycarbonyl)-L-phenylalanine were dissolved in 10 ml of DMF and admixed with 457 mg (3.77 mmol) of N-methylbenzylamine, 2150 mg (5.65 mmol) of O-(7-azabenzotriazol-1-yl)-N,N,N′,N′-tetramethyluronium hexafluorophosphate and 657 μl of N,N-diisopropylethylamine. The reaction mixture was stirred at RT for 30 min and then concentrated under reduced pressure. The residue was taken up in dichloromethane and extracted by shaking three times with water. The organic phase was dried over magnesium sulphate and concentrated. The residue was purified by flash chromatography on silica gel with 3:1 petroleum ether/ethyl acetate as the eluent. The product fractions were concentrated and the residue was dried under high vacuum. This gave 1110 mg (75% of theory) of the Boc-protected intermediate N-benzyl-N^{α}-(tert-butoxycarbonyl)-N-methyl-L-phenylalaninamide.

HPLC (Method 6): Rₜ=2.1 min;

LC-MS (Method 1): Rₜ=1.14 min; MS (ESIpos): m/z=369 (M+H)⁺.

1108 mg (3,007 mmol) of this intermediate were taken up in 30 ml of dichloromethane, 10 ml of trifluoroacetic acid were added, and the mixture was stirred at RT for 30 min Subsequently, the reaction mixture was concentrated under reduced pressure, the remaining residue was stirred with dichloromethane and the solvent was distilled off. The residue was stirred twice more with pentane, the solvent was decanted off again each time and the of the title compound was finally dried under high vacuum. 1075 mg (93% of theory) of the title compound were thus obtained as a resin.

HPLC (Method 6): Rₜ=1.6 min;

LC-MS (Method 1): Rₜ=0.6 min; MS (ESIpos): m/z=269 (M+H)⁺.

### Intermediate 18

### N-benzyl-N^{α}-{(2R,3R)-3-methoxy-2-methyl-3-[(2S)-pyrrolidin-2-yl]propanoyl}-N-methyl-L-phenylalaninamide trifluoroacetate

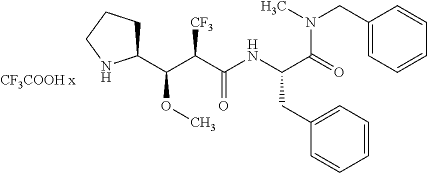 [SMILES: CO[C@@H]([C@@H]1CCCN1)[C@H](C(=O)N[C@@H](Cc1ccccc1)C(=O)N(C)Cc1ccccc1)C(F)(F)F]

First, (2R,3R)-3-[(2S)-1-(tert-butoxycarbonyl)pyrrolidin-2-yl]-3-methoxy-2-methylpropanoic acid (Starting Compound 1) was released from 141 mg (0.491 mmol) of its dicyclohexylamine salt by taking it up in ethyl acetate and extractive shaking with 5% aqueous sulphuric acid. The organic phase was dried over magnesium sulphate, filtered and concentrated. The residue was taken up in 10 ml of DMF and 187.6 mg (0.49 mmol) of N-benzyl-N-methyl-L-phenylalaninamide trifluoroacetate (Intermediate 9), 190.3 mg (1.47 mmol) of O-(7-azabenzotriazol-1-yl)-N,N,N′,N′-tetramethyluronium hexafluorophosphate (HATU) and 256 μl of N,N-diisopropylethylamine were added. The mixture was stirred at RT for 1 h. The reaction mixture was then concentrated, the residue was taken up in ethyl acetate and the solution was subsequently extracted by shaking successively with saturated ammonium chloride solution, saturated sodium hydrogencarbonate solution and water. The organic phase was dried over magnesium sulphate and concentrated. The residue was purified by flash chromatography on silica gel with 30:1 acetonitrile/water as the eluent. The product fractions were concentrated and the residue was dried under high vacuum. This gave 168 mg (64% of theory) of the Boc-protected intermediate tert-butyl (2S)-2-[(1R,2R)-3-({(2S)-1-[benzyl(methyl)amino]-1-oxo-3-phenylpropan-2-yl}amino)-1-methoxy-2-methyl-3-oxopropyl]pyrrolidine-1-carboxylate.

HPLC (Method 6): Rₜ=2.2 min;

LC-MS (Method 2): Rₜ=1.22 min; MS (ESIpos): m/z=538 (M+H)⁺.

168 mg (0.312 mmol) of this intermediate were taken up in 15 ml of dichloromethane, 3 ml of trifluoroacetic acid were added, and the mixture was stirred at RT for 30 min Subsequently, the reaction mixture was concentrated under reduced pressure. The remaining residue was stirred first with dichloromethane, then with diethyl ether, and the solvent was distilled off again each time. After drying under high vacuum, 170 mg (99% of theory) of the title compound were obtained as a resin.

HPLC (Method 6): Rₜ=1.7 min;

LC-MS (Method 1): Rₜ=0.73 min; MS (ESIpos): m/z=438 (M+H)⁺.

### Intermediate 19

### methyl N-{(2R,3R)-3-methoxy-2-methyl-3-[(2S)-pyrrolidin-2-yl]propanoyl}-L-phenylalaninate trifluoroacetate

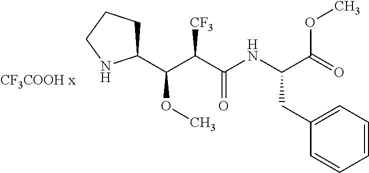 [SMILES: COC(=O)[C@H](Cc1ccccc1)NC(=O)[C@@H]([C@@H](OC)[C@@H]1CCCN1)C(F)(F)F]

The title compound was prepared in analogy to the synthesis of Intermediate 18, proceeding from (2R,3R)-3-[(2S)-1-(tert-butoxycarbonyl)pyrrolidin-2-yl]-3-methoxy-2-methylpropanoic acid (Starting Compound 1), which was released from the dicyclohexylamine salt, and methyl L-phenylalaninate hydrochloride.

HPLC (Method 5): Rₜ=0 6 min;

LC-MS (Method 3): Rₜ=1.17 min; MS (ESIpos): m/z=349 (M+H)⁺.

### Intermediate 20

### benzyl N-{(2R,3R)-3-methoxy-2-methyl-3-[(2S)-pyrrolidin-2-yl]propanoyl}-L-tryptophanate trifluoroacetate

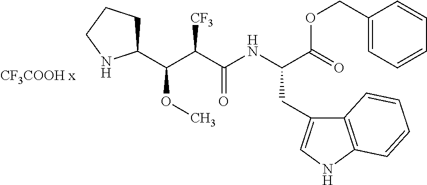 [SMILES: CO[C@@H]([C@@H]1CCCN1)[C@H](C(=O)N[C@@H](Cc1c[nH]c2ccccc12)C(=O)OCc1ccccc1)C(F)(F)F]

The title compound was prepared in analogy to the synthesis of Intermediate 18, proceeding from (2R,3R)-3-[(2S)-1-(tert-butoxy carbonyl)pyrrolidin-2-yl]-3-methoxy-2-methylpropanoic acid (Starting Compound 1), which was released from the dicyclohexylamine salt, and benzyl L-tryptophanate.

HPLC (Method 6): Rₜ=2.0 min;

LC-MS (Method 1): Rₜ=0.8 min; MS (ESIpos): m/z=464 (M+H)⁺.

### Intermediate 21

### benzyl (1S,2R)-1-({(2R,3R)-3-methoxy-2-methyl-3-[(2S)-pyrrolidin-2-yl]propanoyl}amino)-2-phenylcyclopropanecarboxylate trifluoroacetate

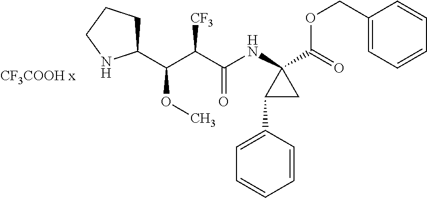 [SMILES: CO[C@@H]([C@@H]1CCCN1)[C@H](C(=O)N[C@@]1(C(=O)OCc2ccccc2)C[C@@H]1c1ccccc1)C(F)(F)F]

The title compound was prepared in analogy to the synthesis of Intermediate 18, proceeding from (2R,3R)-3-[(2S)-1-(tert-butoxycarbonyl)pyrrolidin-2-yl]-3-methoxy-2-methylpropanoic acid (Starting Compound 1), which was released from the dicyclohexylamine salt, and benzyl (1S,2R)-1-amino-2-phenylcyclopropanecarboxylate. Benzyl (1S,2R)-1-amino-2-phenylcyclopropanecarboxylate had been prepared beforehand by standard methods, by esterifying commercially available (1S,2R)-1-[(tert-butoxycarbonyl)amino]-2-phenylcyclopropanecarboxylic acid with benzyl alcohol and subsequent Boc detachment with trifluoroacetic acid.

HPLC (Method 5): Rₜ=1.5 min;

LC-MS (Method 2): Rₜ=0.93 min; MS (ESIpos): m/z=437 (M+H)⁺.

### Intermediate 22

### 6-(2,5-dioxo-2,5-dihydro-1H-pyrrol-1-yl)-N′-methylhexanehydrazide trifluoroacetate

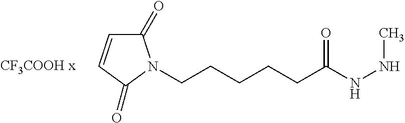 [SMILES: CNNC(=O)CCCCCN1C(=O)C=CC1=O]

100 mg (473 μmol) of 6-(2,5-dioxo-2,5-dihydro-1H-pyrrol-1-yl)hexanoic acid were dissolved in 71 μl of DMF and then admixed with 139 mg (947 μmol) of tert-butyl 1-methylhydrazinecarboxylate, 182 mg (947 μmol) of 1-(3-dimethylaminopropyl)-3-ethylcarbodiimide hydrochloride and 145 mg (947 μmol) of 1-hydroxy-1H-benzotriazole hydrate. The mixture was stirred at RT overnight and then concentrated under reduced pressure. The remaining residue was purified by means of preparative HPLC. After lyophilization from dioxane/water, 129 mg (80% of theory) of the protected intermediate were obtained as a colourless foam.

Subsequently, the 129 mg (380 μmol) were deblocked with 2 ml of trifluoroacetic acid in 8 ml of dichloromethane. After stirring at RT for 1 h, the reaction mixture was concentrated under reduced pressure. The residue was lyophilized from acetonitrile/water, which left 125 mg (83% of theory) of the title compound as a colourless foam.

LC-MS (Method 1): Rₜ=0.38 min; MS (ESIpos): m/z=240 (M+H)⁺

### Intermediate 23

### N-(2-aminoethyl)-4-(2,5-dioxo-2,5-dihydro-1H-pyrrol-1-yl)-N-methylbutanamide trifluoroacetate

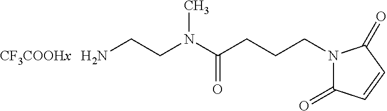 [SMILES: CN(CCN)C(=O)CCCN1C(=O)C=CC1=O]

First, 35 mg (164 μmol) of tert-butyl 2-(methylamino)ethyl carbamate hydrochloride trifluoroacetate, 30 mg (164 μmol) of 4-(2,5-dioxo-2,5-dihydro-1H-pyrrol-1-yl)butanoic acid, 75 mg (197 μmol) of O-(7-azabenzotriazol-1-yl)-N,N,N′,N′-tetramethyluronium hexafluorophosphate and 57 μl of N,N-diisopropylethylamine were combined in 5 ml of DMF and stirred at RT overnight. Subsequently, the solvent was removed under reduced pressure and the remaining residue was purified by means of preparative HPLC. The corresponding fractions were concentrated and, by lyophilization from dioxane/water, 35 mg (63% of theory) of the protected intermediate were obtained.

HPLC (Method 12): Rₜ=1.6 min;

LC-MS (Method 1): Rₜ=0.71 min; MS (ESIpos): m/z=340 (M+H)⁺.

Subsequently, the entire amount of the protected intermediate was deblocked with 1 ml of trifluoroacetic acid in 5 ml of dichloromethane to obtain 28 mg (77% of theory) of the title compound.

LC-MS (Method 3): Rₜ=0.75 min; MS (ESIpos): m/z=240 (M+H)⁺.

### Intermediate 24

### 4-(2,5-dioxo-2,5-dihydro-1H-pyrrol-1-yl)-N-[2-(methylamino)ethyl]butanamide trifluoroacetate

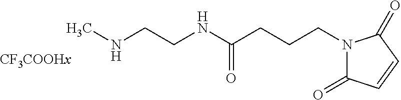 [SMILES: CNCCNC(=O)CCCN1C(=O)C=CC1=O]

First, 35 mg (164 μmol) of tert-butyl(2-aminoethyl)methyl carbamate hydrochloride trifluoroacetate, 30 mg (164 μmol) of 4-(2,5-dioxo-2,5-dihydro-1H-pyrrol-1-yl)butanoic acid, 75 mg (197 μmol) of O-(7-azabenzotriazol-1-yl)-N,N,N′,N′-tetramethyluronium hexafluorophosphate and 57 μl of N,N-diisopropylethylamine were combined in 5 ml of DMF and stirred at RT for 30 min Subsequently, the solvent was removed under reduced pressure and the remaining residue was purified by means of preparative HPLC. The corresponding fractions were concentrated and, by lyophilization from dioxane/water, 51 mg (91% of theory) of the protected intermediate were obtained.

HPLC (Method 12): Rₜ=1.6 min;

LC-MS (Method 1): Rₜ=0.77 min; MS (ESIpos): m/z=340 (M+H)⁺.

Subsequently, the entire amount was deprotected with 1 ml of trifluoroacetic acid in 5 ml of dichloromethane to obtain 45 mg (69% of theory) of the title compound.

LC-MS (Method 1): Rₜ=0.19 min; MS (ESIpos): m/z=240 (M+H)⁺.

### Intermediate 25

### benzyl (2R,3R)-3-methoxy-2-methyl-3-[(2S)-pyrrolidin-2-yl]propanoate trifluoroacetate

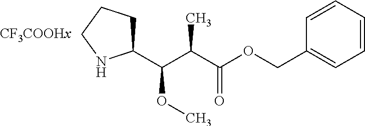 [SMILES: CO[C@@H]([C@@H]1CCCN1)[C@@H](C)C(=O)OCc1ccccc1]

First, (2R,3R)-3-[(2S)-1-(tert-butoxycarbonyl)pyrrolidin-2-yl]-3-methoxy-2-methylpropanoic acid was released from 1.82 g (388 mmol) of its dicyclohexylamine salt by taking it up in ethyl acetate and extractive shaking with 100 ml of 0.5% sulphuric acid. The organic phase was dried over magnesium sulphate, filtered and concentrated. The residue was taken up in 10 ml of dioxane and 10 ml of water, 1517 mg (4.66 mmol) of caesium carbonate were added, and the mixture was treated in an ultrasound bath for 5 min and concentrated under reduced pressure and redistilled once with DMF. The residue was then taken up in 15 ml of dichloromethane, and 1990 mg (11.64 mmol) of benzyl bromide were added. The mixture was treated in an ultrasound bath for 15 min and then concentrated under reduced pressure. The residue was partitioned between ethyl acetate and water, and the organic phase was removed and extracted by shaking with saturated sodium chloride solution and then concentrated. The residue was then purified by preparative HPLC. This gave 1170 mg (80% of theory) of the Boc-protected intermediate.

Subsequently, these 1170 mg were deprotected immediately with 5 ml of trifluoroacetic acid in 15 ml of dichloromethane. After stirring at RT for 15 min, the reaction mixture was concentrated under reduced pressure. The residue was lyophilized from dioxane. After drying under high vacuum, there remained 1333 mg (84% of theory) of the title compound as a yellow oil.

HPLC (Method 6): Rₜ=1.5 min;

LC-MS (Method 1): Rₜ=0.59 min; MS (ESIpos): m/z=278 (M+H)⁺.

### Intermediate 26

### N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide

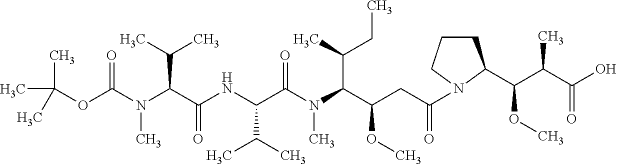 [SMILES: CC[C@H](C)[C@@H]([C@@H](CC(=O)N1CCC[C@H]1[C@H](OC)[C@@H](C)C(=O)O)OC)N(C)C(=O)[C@@H](NC(=O)[C@H](C(C)C)N(C)C(=O)OC(C)(C)C)C(C)C]

1200 mg (2.33 mmol) of N-(tert-butoxycarbonyl)-N-methyl-L-valyl-N-[(2R,3S,4S)-1-carboxy-2-methoxy-4-methylhexan-3-yl]-N-methyl-L-valinamide (Intermediate 5) were combined with 910.8 mg (2.33 mmol) of benzyl (2R,3R)-3-methoxy-2-methyl-3-[(2S)-pyrrolidin-2-yl]propanoate trifluoroacetate (Intermediate 14), 1327 mg (3.49 mmol) of O-(7-azabenzotriazol-1-yl)-N,N,N′,N′-tetramethyluronium hexafluorophosphate and 2027 μl of N,N-diisopropylethylamine in 50 ml of DMF, and the mixture was stirred at RT for 5 min Thereafter, the solvent was removed under reduced pressure. The remaining residue was taken up in ethyl acetate and extracted by shaking successively with 5% aqueous citric acid solution and saturated sodium hydrogencarbonate solution. The organic phase was removed and concentrated. The residue was purified by means of preparative HPLC. The product fractions were combined and concentrated, and the residue was dried under high vacuum. This gave 1000 mg (55% of theory) of the benzyl ester intermediate N-(tert-butoxycarbonyl)-N-methyl-L-valyl-N-[(3R,4S,5S)-1-{(2S)-2-[(1R,2R)-3-(benzyloxy)-1-methoxy-2-methyl-3-oxopropyl]pyrrolidin-1-yl}-3-methoxy-5-methyl-1-oxoheptan-4-yl]-N-methyl-L-valinamide as a resin.

LC-MS (Method 1): Rₜ=1.56 min; MS (ESIpos): m/z=775 (M+H)⁺.

The entire amount of this intermediate obtained was taken up in 25 ml of a mixture of methanol and dichloromethane (20:1), and the benzyl ester group was removed by hydrogenation under standard hydrogen pressure with 10% palladium on activated carbon as a catalyst. After stirring at RT for 30 min, the catalyst was filtered off and the filtrate was concentrated under reduced pressure. This gave 803 mg (91% of theory) of the title compound as a white solid.

HPLC (Method 6): Rₜ=2.1 min;

LC-MS (Method 1): Rₜ=1.24 min; MS (ESIpos): m/z=685 (M+H)⁺.

### Intermediate 27

### (1S,2R)-1-amino-2-phenyl-N-propylcyclopropanecarboxamide trifluoroacetate

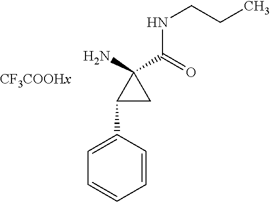 [SMILES: CCCNC(=O)[C@]1(N)C[C@@H]1c1ccccc1]

The title compound was prepared by coupling commercially available (1S,2R)-1-[(tert-butoxycarbonyl)amino]-2-phenylcyclopropanecarboxylic acid with n-propylamine in the presence of O-(7-azabenzotriazol-1-yl)-N,N,N′,N′-tetramethyluronium hexafluorophosphate (HATU) and subsequent Boc detachment with trifluoroacetic acid (yield: 85% of theory over both stages).

HPLC (Method 6): Rₜ=1.2 min;

LC-MS (Method 1): Rₜ=0.52 min; MS (ESIpos): m/z=219 (M+H)⁺.

### Intermediate 28

### ethyl (1S,2R)-1-amino-2-phenylcyclopropanecarboxylate trifluoroacetate

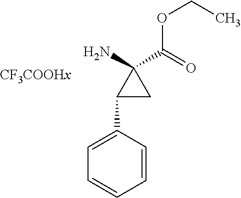

The title compound was prepared by standard methods, by esterifying commercially available (1S,2R)-1-[(tert-butoxycarbonyl)amino]-2-phenylcyclopropanecarboxylic acid with ethanol and subsequent Boc detachment with trifluoroacetic acid.

LC-MS (Method 1): Rₜ=0.50 min; MS (ESIpos): m/z=206 (M+H)+.

### Intermediate 29

### 4-(2,5-dioxo-2,5-dihydro-1H-pyrrol-1-yl)-2,2-dimethylbutanoic acid

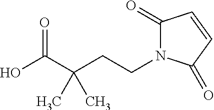

To a solution of 1.39 g (8.95 mmol) of N-methoxycarbonylmaleimide in 44 ml of saturated sodium hydrogencarbonate solution were added, at 0° C., 1.5 g (8.95 mmol) of 4-amino-2,2-dimethylbutyric acid, and the mixture was stirred for 40 min Subsequently, the cooling bath was removed and the reaction mixture was stirred for a further 1 h. While cooling with ice, the reaction mixture was then adjusted to pH 3 by adding sulphuric acid, and extracted with ethyl acetate. The combined organic phases were dried over magnesium sulphate and concentrated. 1.17 g (79% purity, 49% of theory) of the title compound were obtained.

LC-MS (Method 1): Rₜ=0.64 min; m/z=212 (M+H)⁺.

### Intermediate 30

### tert-butyl 2-[4-(2,5-dioxo-2,5-dihydro-1H-pyrrol-1-yl)-2,2-dimethylbutanoyl]hydrazinecarboxylate

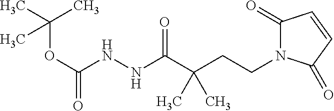 [SMILES: CC(C)(C)OC(=O)NNC(=O)C(C)(C)CCN1C(=O)C=CC1=O]

To a solution of 50 mg (237 μmol) of 4-(2,5-dioxo-2,5-dihydro-1H-pyrrol-1-yl)-2,2-dimethylbutanoic acid in 2 ml of THF were added, at 0° C., first 26 μl (237 μmol) of 4-methylmorpholine and then 31 μl (237 μmol) of isobutyl chloroformate. After removing the cooling bath and stirring at RT for a further 15 min, 31.3 mg (237 μmol) of tert-butyloxycarbonyl hydrazide were added. The reaction mixture was stirred overnight and then concentrated. The residue was purified by preparative HPLC. 50.8 mg (66% of theory) of the title compound were obtained.

LC-MS (Method 1): Rₜ=0.71 min; m/z=324 (M−H)⁻.

### Intermediate 31

### 4-(2,5-dioxo-2,5-dihydro-1H-pyrrol-1-yl)-2,2-dimethylbutanehydrazide trifluoroacetate

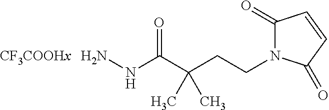 [SMILES: CC(C)(CCN1C(=O)C=CC1=O)C(=O)NN]

50 mg (154 mmol) of tert-butyl 2-[4-(2,5-dioxo-2,5-dihydro-1H-pyrrol-1-yl)-2,2-dimethylbutanoyl]hydrazinecarboxylate were dissolved in 2 ml of dichloromethane, and 0.4 ml of trifluoroacetic acid was added. The reaction mixture was stirred at RT for 30 min and then concentrated. 55.2 mg (93% purity, 99% of theory) of the title compound were obtained.

LC-MS (Method 1): Rₜ=0.36 min; m/z=226 (M+H)⁺.

### Intermediate 32

### adamantan-1-ylmethyl N-(tert-butoxycarbonyl)-L-phenylalaninate

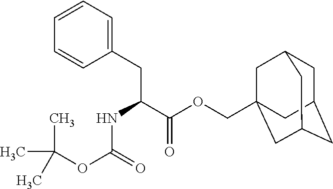 [SMILES: CC(C)(C)OC(=O)N[C@@H](Cc1ccccc1)C(=O)OCC12CC3CC(CC(C3)C1)C2]

To a solution of 500 mg (1.89 mmol) of N-Boc-L-phenylalanine in 25 ml of dichloromethane were added, at RT, 1192 mg (6.2 mmol) of EDC, 578 μl (4.1 mmol) of triethylamine, 345 mg (2.8 mmol) of DMAP and 345 mg (2.1 mmol) of 1-adamantylmethanol. The reaction mixture was stirred overnight, then diluted with 50 ml of dichloromethane, and was successively washed with 10% aqueous citric acid solution, water and saturated sodium chloride solution. The organic phase was dried over magnesium sulphate, then concentrated, and the residue was purified by preparative HPLC. 769 mg (90% of theory) of the title compound were obtained.

LC-MS (Method 2): Rₜ=1.84 min; m/z=414 (M+H)⁺.

### Intermediate 33

### adamantan-1-ylmethyl L-phenylalaninate hydrochloride

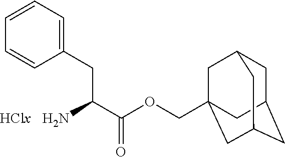 [SMILES: N[C@@H](Cc1ccccc1)C(=O)OCC12CC3CC(CC(C3)C1)C2]

769 mg (1.86 mmol) of adamantan-1-ylmethyl N-(tert-butoxycarbonyl)-L-phenylalaninate (Intermediate 13) were dissolved in 25 ml of a 4 N solution of hydrogen chloride in dioxane and stirred at RT for 1 h. Subsequently, the reaction mixture was concentrated and the residue was dried under reduced pressure. 619 mg (95% of theory) of the title compound were obtained.

LC-MS (Method 1): Rₜ=0.82 min; m/z=314 (M+H)⁺.

### Intermediate 34

### N-(tert-butoxycarbonyl)-N-methyl-L-valyl-N-[(3R,4S,5S)-1-{(2S)-2-[(1R,2R)-3-{[(2S)-1-(adamantan-1-ylmethoxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

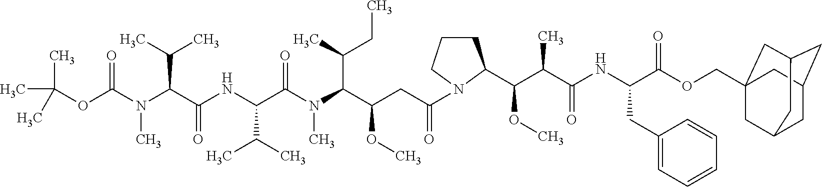 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCC12CC3CC(CC(C3)C1)C2)OC)N(C)C(=O)[C@@H](NC(=O)[C@H](C(C)C)N(C)C(=O)OC(C)(C)C)C(C)C]

To a solution of 20 mg (29 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide in 1 ml of DMF were added, at RT, 15.3 μl (88 μmol) of N,N-diisopropylethylamine, 6.7 mg (44 μmol) of HOBt and 6.7 mg (35 μmol) of EDC, and the mixture was stirred for 30 min Subsequently, 10.1 mg (32 μmol) of adamantan-1-yl L-phenylalaninate hydrochloride were added. After stirring overnight, the reaction mixture was separated directly into its components via preparative HPLC. 27.5 mg (93% of theory) of the title compound were obtained.

LC-MS (Method 1): Rₜ=1.70 min; m/z=980 (M+H)⁺.

### Intermediate 35

### N-methyl-L-valyl-N-[(3R,4S,5S)-1-{(2S)-2-[(1R,2R)-3-{[(2S)-1-(adamantan-1-ylmethoxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoro acetate

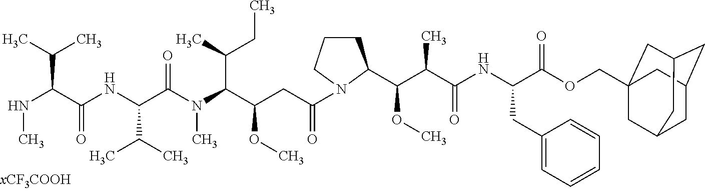 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCC12CC3CC(CC(C3)C1)C2)OC)N(C)C(=O)[C@@H](NC(=O)[C@@H](NC)C(C)C)C(C)C]

27.5 mg (28 μmol) of N-(tert-butoxycarbonyl)-N-methyl-L-valyl-N-[(3R,4S,5S)-1-{(2S)-2-[(1R,2R)-3-{[(2S)-1-(adamantan-1-ylmethoxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 1.8 ml of dichloromethane, and 361 μl of TFA were added. The reaction mixture was stirred for 30 min and then concentrated. The residue was taken up in water and lyophilized. 22.7 mg (81% of theory) of the title compound were obtained.

LC-MS (Method 1): Rₜ=1.14 min; m/z=880 (M+H)⁺.

### Intermediate 36

### tert-butyl (2S)-1-(benzyloxy)-3-phenylpropan-2-yl carbamate

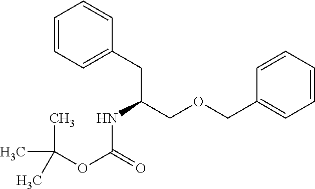 [SMILES: CC(C)(C)OC(=O)N[C@H](COCc1ccccc1)Cc1ccccc1]

Under an argon atmosphere, 500 mg (1.99 mmol) of N-Boc-L-phenylalaninol were dissolved in 5 ml of DMF and cooled to 0° C. Subsequently, 159 mg (3.98 mmol) of a 60% suspension of sodium hydride in paraffin oil were added. The reaction mixture was stirred until the evolution of gas had ended and then 260 μl (2.19 mmol) of benzyl bromide were added. The cooling bath was removed and the reaction mixture was stirred at RT for 2 h. Thereafter, the reaction mixture was concentrated, the residue was taken up in ice-water and the mixture was extracted with dichloromethane. The organic phase was washed with saturated sodium chloride solution, dried over magnesium sulphate and concentrated. The residue was purified by means of preparative HPLC. 226 mg (33% of theory) of the title compound were obtained.

LC-MS (Method 1): Rₜ=1.28 min; m/z=342 (M+H)⁺.

### Intermediate 37

### (2S)-1-(benzyloxy)-3-phenylpropan-2-amine hydrochloride

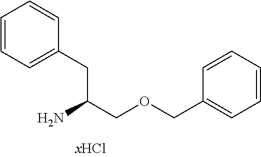 [SMILES: N[C@H](COCc1ccccc1)Cc1ccccc1]

220 mg (644 μmol) of tert-butyl (2S)-1-(benzyloxy)-3-phenylpropan-2-yl carbamate were dissolved in 11 ml of a 4 N solution of hydrogen chloride in dioxane and stirred at RT for 1 h. Then the reaction mixture was concentrated and the residue was dried under reduced pressure. 138 mg (77% of theory) of the title compound were obtained.

LC-MS (Method 1): Rₜ=0.65 min; m/z=242 (M+H)⁺.

### Intermediate 38

### N-(tert-butoxycarbonyl)-N-methyl-L-valyl-N-[(3R,4S,5S)-1-{(2S)-2-[(1R,2R)-3-{[(2S)-1-(benzyloxy)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

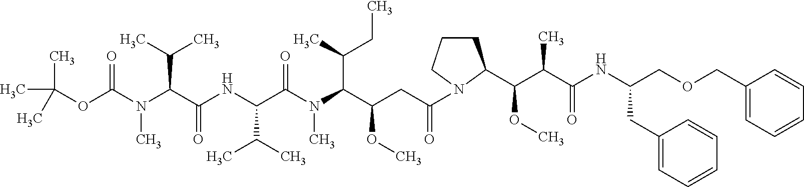 [SMILES: CC[C@H](C)[C@@H]([C@@H](CC(=O)N1CCC[C@H]1[C@H](OC)[C@@H](C)C(=O)N[C@H](COCc1ccccc1)Cc1ccccc1)OC)N(C)C(=O)[C@@H](NC(=O)[C@H](C(C)C)N(C)C(=O)OC(C)(C)C)C(C)C]

To a solution of 20 mg (29 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide in 1 ml of DMF were added, at RT, 15.3 μl (88 μmol) of N,N-diisopropylethylamine, 6.7 mg (44 μmol) of HOBt and 6.7 mg (35 μmol) of EDC, and the mixture was stirred for 30 min Subsequently, 7.8 mg (32 μmol) of (2S)-1-(benzyloxy)-3-phenylpropan-2-amine hydrochloride were added. After stirring overnight, the reaction mixture was separated directly into its components via preparative HPLC. 26 mg (98% of theory) of the title compound were obtained.

LC-MS (Method 1): Rt=1.51 min; m/z=909 (M+H)+.

### Intermediate 39

### N-methyl-L-valyl-N-[(3R,4S,5S)-1-{(2S)-2-[(1R,2R)-3-{[(2S)-1-(benzyloxy)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

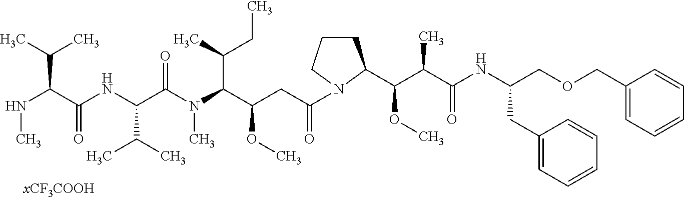 [SMILES: CC[C@H](C)[C@@H]([C@@H](CC(=O)N1CCC[C@H]1[C@H](OC)[C@@H](C)C(=O)N[C@H](COCc1ccccc1)Cc1ccccc1)OC)N(C)C(=O)[C@@H](NC(=O)[C@@H](NC)C(C)C)C(C)C]

26 mg (29 μmol) of N-(tert-butoxycarbonyl)-N-methyl-L-valyl-N-[(3R,4S,5S)-1-{(2S)-2-[(1R,2R)-3-{[(2S)-1-(benzyloxy)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 1.8 ml of dichloromethane, and 370 μl of TFA were added. The reaction mixture was stirred at RT for 30 min and then concentrated. The residue was taken up in water and lyophilized. 26.4 mg (quant.) of the title compound were obtained.

LC-MS (Method 1): Rₜ=0.97 min; m/z=809 (M+H)⁺.

### Intermediate 40

### N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

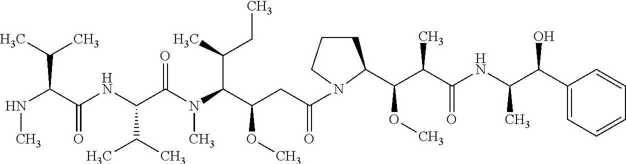 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

50 mg (70 μmol) of Intermediate 26 and 11 mg (70 μmol) of (1S,2R)-2-amino-1-phenylpropan-1-ol in 10 ml of DMF were admixed with 42 mg (0.11 μmol) of O-(7-azabenzotriazol-1-yl)-N,N,N′,N′-tetramethyluronium hexafluorophosphate and 25 μl of N,N-diisopropylethylamine, and the reaction mixture was stirred at RT for 5 min. This was followed by concentration and purification of the residue by means of preparative HPLC. After combining the corresponding fractions, concentrating and drying under high vacuum, 49 mg (81%) of the protected intermediate were obtained. Subsequently, the Boc group was detached by known conditions with trifluoroacetic acid in dichloromethane. Concentration was followed by the purification of the title compound by preparative HPLC, and 26 mg (52%) of the title compound were obtained.

HPLC (Method 12): Rₜ=1.65 min;

LC-MS (Method 1): Rₜ=0.77 min; MS (ESIpos): m/z=718 (M+H)⁺.

### Intermediate 41

### 3-{2-[2-(2-aminoethoxy)ethoxy]ethoxy}propanoic acid trifluoroacetate

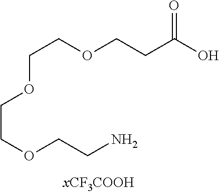 [SMILES: NCCOCCOCCOCCC(=O)O]

150 mg (541 μmol) of tert-butyl 3-{2-[2-(2-aminoethoxy)ethoxy]ethoxy}propanoate were dissolved in 3 ml of dichloromethane, 1.5 ml of trifluoroacetic acid were added, and the reaction mixture was stirred at RT for 1 h, then concentrated.

181 mg (100% of theory) of the title compound were obtained.

MS (EI): m/z 222 (M+H)⁺

### Intermediate 42

### 3-(2-{2-[2-(2,5-dioxo-2,5-dihydro-1H-pyrrol-1-yl)ethoxy]ethoxy}ethoxy)propanoic acid

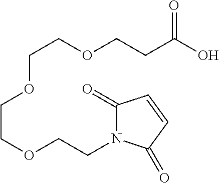 [SMILES: O=C(O)CCOCCOCCOCCN1C(=O)C=CC1=O]

186 mg (555 μmol) of 3-{2-[2-(2-aminoethoxy)ethoxy]ethoxy}propanoic acid trifluoroacetate were dissolved in 2.6 ml of saturated sodium hydrogencarbonate solution and admixed at 0° C. with 86 mg (555 μmol) of N-methoxycarbonylmaleimide. The reaction mixture was stirred at 0° C. for 40 min and at RT for 1 h, then cooled again to 0° C., adjusted to pH 3 with sulphuric acid and extracted 3× with 25 ml of ethyl acetate. The combined organic phases were dried over magnesium sulphate and concentrated.

126 mg (75% of theory) of the title compound were obtained.

LC-MS (Method 1): Rₜ=0.53 min; m/z=302 (M+H)⁺.

### Intermediate 43

### tert-butyl 15-(2,5-dioxo-2,5-dihydro-1H-pyrrol-1-yl)-4-oxo-7,10,13-trioxa-2,3-diazapentadecan-1-oate

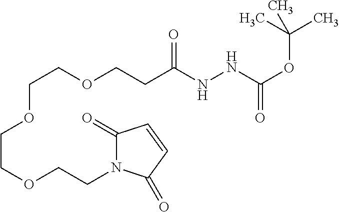 [SMILES: CC(C)(C)OC(=O)NNC(=O)CCOCCOCCOCCN1C(=O)C=CC1=O]

125 mg (417 μmol) of 3-(2-{2-[2-(2,5-dioxo-2,5-dihydro-1H-pyrrol-1-yl)ethoxy]ethoxy}ethoxy) propanoic acid were dissolved at 0° C. in 2.1 ml of THF and admixed with 46 μl (417 mmol) of 4-methylmorpholine and 54.5 μl (417 μmol) of isobutyl chloroformate. The ice bath was removed and the reaction mixture was stirred at RT for 30 min Subsequently, at 0° C., 55 mg (417 μmol) of tert-butyloxycarbonyl hydrazide were added. The reaction mixture was warmed to RT overnight, concentrated and purified by preparative HPLC.

60 mg (33% of theory) of the title compound were obtained.

LC-MS (Method 1): Rₜ=0.66 min; m/z=416 (M+H)⁺.

### Intermediate 44

### 3-(2-{2-[2-(2,5-dioxo-2,5-dihydro-1H-pyrrol-1-yl)ethoxy]ethoxy}ethoxy)propanehydrazide trifluoroacetate

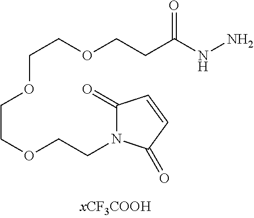 [SMILES: NNC(=O)CCOCCOCCOCCN1C(=O)C=CC1=O]

60 mg (145 μmol) of tert-butyl 15-(2,5-dioxo-2,5-dihydro-1H-pyrrol-1-yl)-4-oxo-7,10,13-trioxa-2,3-diazapentadecan-1-oate were dissolved in 1 ml of dichloromethane, and 0.2 ml of trifluoroacetic acid was added. The reaction mixture was stirred at RT for 30 min and then concentrated.

62 mg (100% of theory) of the title compound were obtained.

LC-MS (Method 1): Rₜ=0.35 min; m/z=316 (M+H)⁺.

### Intermediate 45

### benzyl (1S,2R)-1-amino-2-phenylcyclopropanecarboxylate trifluoroacetate

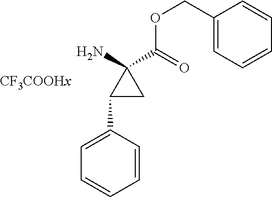 [SMILES: N[C@@]1(C(=O)OCc2ccccc2)C[C@@H]1c1ccccc1]

The title compound was prepared by standard methods, by esterifying commercially available (1S,2R)-1-[(tert-butoxycarbonyl)amino]-2-phenylcyclopropanecarboxylic acid with benzyl alcohol and subsequent Boc detachment with trifluoroacetic acid.

LC-MS (Method 1): Rₜ=0.72 min; MS (ESIpos): m/z=268 (M+H)⁺.

### Intermediate 46

### N-(tert-butoxycarbonyl)-N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide

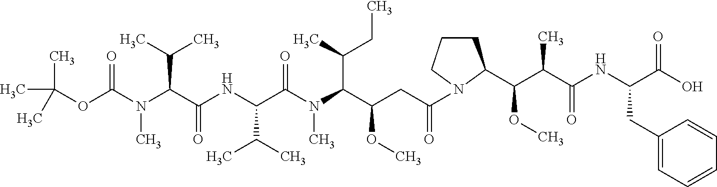 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OC(C)(C)C)C(C)C]

383 mg (0.743 mmol) of N-(tert-butoxy carbonyl)-N-methyl-L-valyl-N-[(2R,3S,4S)-1-carboxy-2-methoxy-4-methylhexan-3-yl]-N-methyl-L-valinamide (Intermediate 8) were combined with 485 mg (0.743 mmol) of benzyl N-{(2R,3R)-3-methoxy-2-methyl-3-[(2S)-pyrrolidin-2-yl]propanoyl}-L-phenylalaninate trifluoroacetate (Intermediate 12), 424 mg (1.114 mmol) of O-(7-azabenzotriazol-1-yl)-N,N,N′,N′-tetramethyluronium hexafluorophosphate and 388 μl of N,N-diisopropylethylamine in 15 ml of DMF, and the mixture was stirred at RT for 10 min Subsequently, the solvent was removed under reduced pressure. The remaining residue was taken up in ethyl acetate and extracted by shaking successively with 5% aqueous citric acid solution and saturated sodium hydrogencarbonate solution. The organic phase was removed and concentrated, and the residue was purified by means of preparative HPLC. The product fractions were combined and concentrated, and the residue was dried under high vacuum. 335 mg (48% of theory) of the benzyl ester intermediate were obtained as a foam.

LC-MS (Method 1): Rₜ=1.49 min; MS (ESIpos): m/z=922 (M+H)⁺.

100 mg (0.11 mmol) of this intermediate were taken up in 15 ml of methanol and the benzyl ester group was removed by hydrogenation under standard hydrogen pressure with 10% palladium on activated carbon as a catalyst. After stirring at RT for 1 h, the catalyst was filtered off and the filtrate was concentrated under reduced pressure. After lyophilization from dioxane, 85 mg (94% of theory) of the title compound were obtained as a solid.

HPLC (Method 12): Rₜ=2 4 min;

LC-MS (Method 1): Rₜ=1.24 min; MS (ESIpos): m/z=832 (M+H)⁺.

### Intermediate 47

### N-benzyl-L-tryptophanamide trifluoroacetate

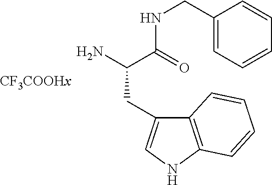 [SMILES: N[C@@H](Cc1c[nH]c2ccccc12)C(=O)NCc1ccccc1]

202 mg (0.5 mmol) of 2,5-dioxopyrrolidin-1-yl N-(tert-butoxycarbonyl)-L-tryptophanate and 45 mg (0.42 mmol) of benzylamine were dissolved in 10 ml of DMF, and 110 μl (630 μmol) of N,N-diisopropylethylamine were added. The reaction mixture was stirred at RT for 3 h. Subsequently, the reaction mixture was concentrated under reduced pressure and the residue was purified by flash chromatography on silica gel (eluent: 20:0.5:0.05 dichloromethane/methanol/17% aq ammonia). The corresponding fractions were combined and concentrated. The resulting residue was digested with diethyl ether and then dried under high vacuum. Subsequently, this residue was taken up in 10 ml of dichloromethane, and 3 ml of anhydrous trifluoroacetic acid were added. After stirring at RT for 45 minutes, the mixture was concentrated and the residue was purified by preparative HPLC. After drying under high vacuum, 117 mg (57% of theory over both stages) of the title compound were obtained.

HPLC (Method 12): Rₜ=1.6 min;

LC-MS (Method 1): Rₜ=0.66 min; MS (ESIpos): m/z=294 (M+H)⁺.

### Intermediate 48

### (1S,2R)-1-amino-2-phenylcyclopropanecarboxamide trifluoroacetate

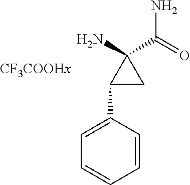 [SMILES: NC(=O)[C@]1(N)C[C@@H]1c1ccccc1]

50 mg (180 μmol) of commercially available (1S,2R)-1-[(tert-butoxycarbonyl)amino]-2-phenylcyclopropanecarboxylic acid were dissolved in 5 ml of DMF, 94 μl (541 μmol) of N,N-diisopropylethylamine, 31 mg (270 μmol) of N-hydroxysuccinimide and 41.5 mg (216 μmol) of EDC were added, and then the mixture was stirred at RT overnight. The reaction mixture was then concentrated, the residue was taken up in dioxane, 71 mg (901 μmol) of ammonium hydrogencarbonate were added, and the reaction mixture was then left to stand at RT for 3 days. The reaction mixture was then diluted with a 1:1 mixture of ethyl acetate and water. The organic phase was removed, dried over magnesium sulphate and concentrated. The resulting residue was subsequently taken up in 3 ml of dichloromethane, and 3 ml of anhydrous trifluoroacetic acid were added. Stirring at RT for 1 h was followed by concentration. The residue was stirred with pentane, filtered off with suction and lyophilized from dioxane. In this way, 32 mg (62% of theory over both stages) of the title compound were obtained.

HPLC (Method 6): Rₜ=0.38 min;

LC-MS (Method 1): Rₜ=0.20 min; MS (ESIpos): m/z=177 (M+H)+.

### Intermediate 49

### N^{α}-{(2R,3R)-3-methoxy-2-methyl-3-[(2S)-pyrrolidin-2-yl]propanoyl}-L-tryptophanamide trifluoroacetate

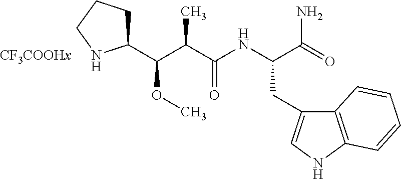 [SMILES: CO[C@@H]([C@@H]1CCCN1)[C@@H](C)C(=O)N[C@@H](Cc1c[nH]c2ccccc12)C(N)=O]

The title compound was prepared in analogy to the synthesis of Intermediate 13 from Starting Compound 1 and L-tryptophanamide hydrochloride.

HPLC (Method 5): Rₜ=1.4 min;

LC-MS (Method 1): Rₜ=0.92 min; MS (ESIpos): m/z=473 (M+H)⁺.

### Intermediate 50

### 4-nitrophenyl 2-(2,5-dioxo-2,5-dihydro-1H-pyrrol-1-yl)ethyl carbamate

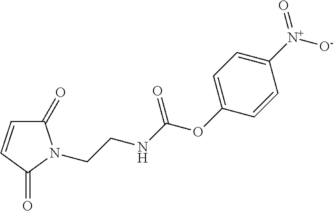 [SMILES: O=C(NCCN1C(=O)C=CC1=O)Oc1ccc([N+](=O)[O-])cc1]

813 mg (3.1 mmol) of triphenylphosphine were dissolved in 25 ml of THF and cooled to −70° C. under argon. After dropwise addition of 627 mg (3.1 mmol) of diisopropyl azodicarboxylate, the mixture was stirred for 5 min Subsequently, 500 mg (3.1 mmol) of tert-butyl 2-aminoethyl carbamate dissolved in 5 ml of THF were added dropwise, and the reaction mixture was stirred at −70° C. for a further 5 min. Then 136.6 mg (1.55 mmol) of 2,2-dimethyl-1-propanol dissolved in 1 ml of THF and 301 mg (3.1 mmol) of maleimide were added, the reaction mixture was stirred at −70° C. for a further 10 min and then the mixture was warmed to RT. After stirring at RT for a further 16 h, the solvent was removed under reduced pressure and the residue was purified by means of preparative HPLC. This gave 463 mg (62%) of the protected intermediate.

After removing the Boc protecting group under standard conditions, 652 mg of 1-(2-aminoethyl)-1H-pyrrole-2,5-dione were obtained as the trifluoroacetate.

112.9 mg (543 μmol) of nitrophenyl chloroformate were dissolved in 30 ml of THF and, after addition of 100 mg (271.6 μmol) of 1-(2-aminoethyl)-1H-pyrrole-2,5-dione trifluoroacetate, the mixture was stirred at RT for 30 min. The mixture was filtered and the filtrate was concentrated to dryness and then slurried with diethyl ether. After filtration with suction and drying, 60 mg (95% of theory) of the title compound were obtained.

HPLC (Method 5): Rt=0.65 min;

LC-MS (Method 1): Rt=0.74 min; MS (ESIpos): m/z=306 (M+H)+.

### Intermediate 51

### (1S)-2-phenyl-1-(5-phenyl-1,3,4-oxadiazol-2-yl)ethanamine trifluoroacetate

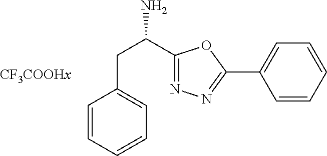 [SMILES: N[C@@H](Cc1ccccc1)c1nnc(-c2ccccc2)o1]

200 mg (0.75 mmol) of N-(tert-butoxycarbonyl)-L-phenylalanine were initially charged at 0° C. in 5.5 ml of dichloromethane, and 128 mg (0.79 mmol) of 1,1′-carbonyldiimidazole were added. After 30 min, 103 mg (0.75 mmol) of benzoyl hydrazide were added. After a further 45 min at 0° C., 500 mg (1.5 mmol) of carbon tetrabromide and 395 mg (1.5 mmol) of triphenylphosphine were finally added. The reaction mixture was stirred first at 0° C. for 2 h and then at RT overnight. The mixture was subsequently concentrated on a rotary evaporator, and the residue was dried under high vacuum. The crude product thus obtained was purified by means of preparative HPLC. 217 mg (78% of theory) of the Boc-protected intermediate tert-butyl (1S)-2-phenyl-1-(5-phenyl-1,3,4-oxadiazol-2-yl)ethyl carbamate were obtained.

LC-MS (Method 12): Rₜ=1.15 min; MS (ESIpos): m/z=366 (M+H)⁺

217 mg (0.59 mmol) of this intermediate were taken up in 3 ml of dichloromethane, 0.6 ml of trifluoroacetic acid were added, and the mixture was stirred at RT for 30 min Subsequently, the reaction mixture was concentrated under reduced pressure. The remaining residue was the reaction mixture dried further under reduced pressure and then lyophilized from dioxane. In this way, 214 mg (90% of theory) of the title compound were obtained.

LC-MS (Method 11): Rₜ=0.62 min; MS (ESIpos): m/z=266 (M+H)⁺

### Intermediate 52

### (1R)-2-phenyl-1-(5-phenyl-1,3,4-oxadiazol-2-yl)ethanamine trifluoroacetate

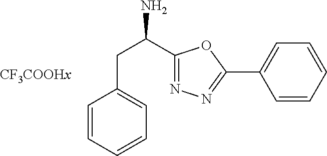 [SMILES: N[C@H](Cc1ccccc1)c1nnc(-c2ccccc2)o1]

200 mg (0.75 mmol) of N-(tert-butoxycarbonyl)-D-phenylalanine were initially charged at 0° C. in 5.5 ml of dichloromethane, and 128.3 mg (0.79 mmol) of 1,1′-carbonyldiimidazole were added. After 30 min, 103 mg (0.75 mmol) of benzoyl hydrazide were added. After a further 45 min at 0° C., 500 mg (1.5 mmol) of carbon tetrabromide and 395 mg (1.5 mmol) of triphenylphosphine were finally added. The reaction mixture was stirred first at 0° C. for 2 h and then at RT overnight. The mixture was subsequently concentrated on a rotary evaporator, and the residue was dried under high vacuum. The crude product thus obtained was purified by means of preparative HPLC. 219 mg (80% of theory) of the Boc-protected intermediate tert-butyl (1R)-2-phenyl-1-(5-phenyl-1,3,4-oxadiazol-2-yl)ethyl carbamate were obtained.

LC-MS (Method 2): Rₜ=1.36 min; MS (ESIpos): m/z=366 (M+H)⁺

219 mg (0.6 mmol) of this intermediate were taken up in 3 ml of dichloromethane, 0.6 ml of trifluoroacetic acid were added, and the mixture was stirred at RT for 30 min Subsequently, the reaction mixture was concentrated under reduced pressure. The remaining residue was the reaction mixture dried further under reduced pressure and then lyophilized from dioxane. In this way, 196 mg (86% of theory) of the title compound were obtained as a solid.

HPLC (Method 10): Rₜ=2.41 min

### Intermediate 53

### (2S)-1-(benzylsulphonyl)-3-phenylpropan-2-amine

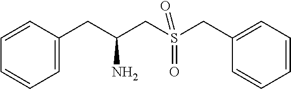 [SMILES: N[C@@H](Cc1ccccc1)CS(=O)(=O)Cc1ccccc1]

200 mg (1.13 mmol) of (4S)-4-benzyl-1,3-oxazolidin-2-one were initially charged in 3 ml of tert-butanol, and 280 mg (2.26 mmol) of benzyl mercaptan were added. The mixture was subsequently heated under reflux for 2 days. Thereafter, the reaction mixture was concentrated on a rotary evaporator and the resulting (2S)-1-(benzylsulphanyl)-3-phenylpropan-2-amine intermediate was converted further directly, without workup.

HPLC (Method 10): Rₜ=2.63 min

LC-MS (Method 1): Rₜ=0.67 min; MS (ESIpos): m/z=258 (M+H)⁺

The crude intermediate obtained above was dissolved in a solution of 2 ml of 30% hydrogen peroxide and 5 ml of formic acid, and the mixture was stirred at RT for 12 h. Then the reaction mixture was added to saturated sodium sulphate solution and extracted three times with ethyl acetate. The organic phase was dried over magnesium sulphate and concentrated under reduced pressure. The crude product obtained was purified by means of preparative HPLC. 343 mg (61% of theory) of the title compound were thus obtained.

HPLC (Method 10): Rₜ=2.40 min;

LC-MS (Method 12): Rₜ=0.65 min; MS (ESIpos): m/z=290 (M+H)⁺

### Intermediate 54

### (2S,3E)-1,4-diphenylbut-3-en-2-amine

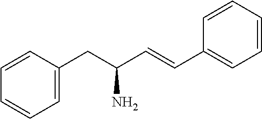 [SMILES: N[C@H](/C=C/c1ccccc1)Cc1ccccc1]

552.7 mg (9.85 mmol) of potassium hydroxide were dissolved in methanol, adsorbed onto 1.1 g of neutral aluminium oxide and then dried under high vacuum. To a solution of 240 mg (0.82 mmol) of (2S)-1-(benzylsulphonyl)-3-phenylpropan-2-amine and 1.56 g of the potassium hydroxide on aluminium oxide thus prepared in 6.2 ml of n-butanol were added dropwise, at 5-10° C., 307 μl (3.3 mmol) of dibromodifluoromethane. The reaction mixture was stirred at RT for 2 h, then filtered through Celite, and the residue was washed thoroughly with dichloromethane. The filtrate was concentrated and the resulting residue was dried under reduced pressure. The crude product thus obtained was purified by means of preparative HPLC. 98 mg (35% of theory) of the title compound were obtained with an EZ diastereomer ratio of 4:1.

HPLC (Method 10): Rₜ=2.46 min;

LC-MS (Method 12): Rₜ=0.75 min; MS (ESIpos): m/z=224 (M+H)⁺

The EZ diastereomer mixture obtained above was dissolved in 2 ml of ethanol and 0.2 ml of N,N-diisopropylethylamine, and separated by means of HPLC on chiral phase [column: Daicel Chiralpak AD-H, 5 μm 250 mm×20 mm, eluent: hexane/(ethanol+0.2% diethylamine) 50:50 v/v; UV detection: 220 nm; temperature: 30° C.]. The appropriate fractions were concentrated on a rotary evaporator, and the residue was dried under reduced pressure. 45 mg of the title compound were obtained.

¹H NMR (400 MHz, DMSO-d₆) δ [ppm]=2.62-2.83 (m, 2H) 3.52-3.71 (m, 1H) 6.18-6.30 (m, 1H) 6.34-6.46 (m, 1H) 6.98-7.57 (m, 10H) [further signals hidden under solvent peaks].

### Intermediate 55

### N-methyl-L-valyl-N-[(3R,4S,5S)-3-methoxy-1-{(2S2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide trifluoroacetate

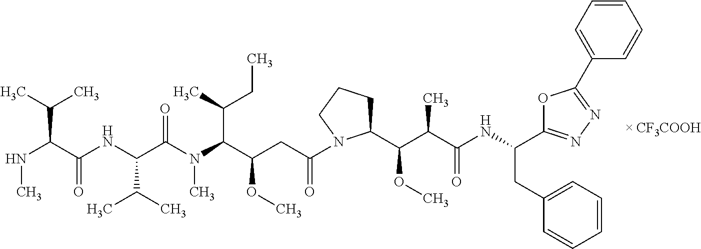 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@@H](NC)C(C)C)C(C)C]

20 mg (29 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide were dissolved in 1 ml of DMF, 13.3 mg (35 μmol) of HATU and 15.3 μl (88 μmol) of N,N-diisopropylethylamine were added, and the mixture was stirred at RT for 30 min Subsequently, 12.2 mg (32 μmol) of (1S)-2-phenyl-1-(5-phenyl-1,3,4-oxadiazol-2-yl)ethanamine trifluoroacetate were added. The reaction mixture was stirred at RT overnight and then separated by preparative HPLC. This gave 22 mg (81% of theory) of N-(tert-butoxycarbonyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

LC-MS (Method 12): Rₜ=1.45 min; MS (ESIpos): m/z=933 (M+H)⁺

By subsequently detaching the BOC protecting group with trifluoroacetic acid, 22.4 mg (98% of theory) of the title compound were then obtained.

LC-MS (Method 11): Rₜ=0.85 min; MS (ESIpos): m/z=833 (M+H)⁺

### Intermediate 56

### N-methyl-L-valyl-N-[(3R,4S,5S)-3-methoxy-1-{(2S)-2-[(1R,2R)-1-methoxy-2-methyl-3-oxo-3-{[(1R)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide trifluoroacetate

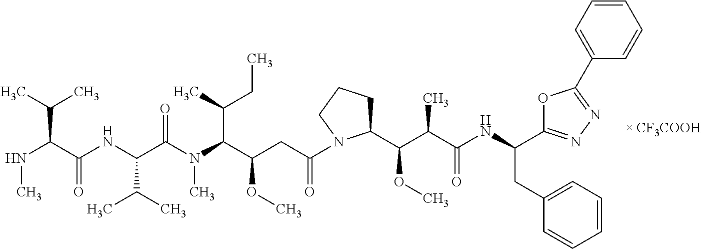 [SMILES: CC[C@H](C)[C@@H]([C@@H](CC(=O)N1CCC[C@H]1[C@H](OC)[C@@H](C)C(=O)N[C@H](Cc1ccccc1)c1nnc(-c2ccccc2)o1)OC)N(C)C(=O)[C@@H](NC(=O)[C@@H](NC)C(C)C)C(C)C]

N-(tert-Butoxycarbonyl)-N-methyl-L-valyl-N-[(3R,4S,5S)-3-methoxy-1-{(2S)-2-[(1R,2R)-1-methoxy-2-methyl-3-oxo-3-{[(1R)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide was prepared in analogy to the synthesis of Intermediate 55, by reaction of 20 mg (29 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide with 12.2 mg (32 μmol) of (1R)-2-phenyl-1-(5-phenyl-1,3,4-oxadiazol-2-yl)ethanamine trifluoroacetate.

Yield: 17 mg (64% of theory)

HPLC (Method 10): Rₜ=3.74 min;

LC-MS (Method 1): Rₜ=1.45 min; MS (ESIpos): m/z=933 (M+H)⁺

By subsequently detaching the BOC protecting group with trifluoroacetic acid, 17.1 mg (99% of theory) of the title compound were then obtained.

HPLC (Method 10): Rₜ=2.55 min;

LC-MS (Method 11): Rₜ=0.85 min; MS (ESIpos): m/z=833 (M+H)⁺

### Intermediate 57

### N-methyl-L-valyl-N-[(3R,4S,5S)-1-{(2S)-2-[(1R,2R)-3-{[(2S)-1-(benzylsulphonyl)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

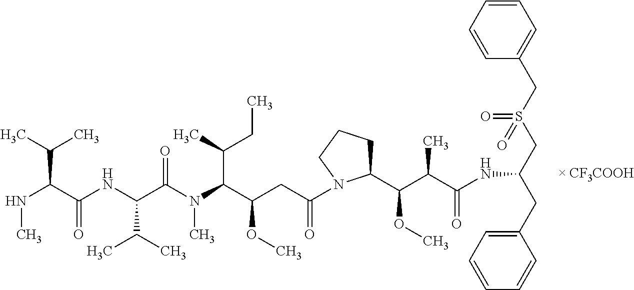 [SMILES: CC[C@H](C)[C@@H]([C@@H](CC(=O)N1CCC[C@H]1[C@H](OC)[C@@H](C)C(=O)N[C@@H](Cc1ccccc1)CS(=O)(=O)Cc1ccccc1)OC)N(C)C(=O)[C@@H](NC(=O)[C@@H](NC)C(C)C)C(C)C]

N-(tert-Butoxycarbonyl)-N-methyl-L-valyl-N-[(3R,4S,5S)-1-{(2S)-2-[(1R,2R)-3-{[(2S)-1-(benzylsulphonyl)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared in analogy to the synthesis of Intermediate 55, by reaction of 20 mg (29 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide with 9.3 mg (20 μmol) of (2S)-1-(benzylsulphonyl)-3-phenylpropan-2-amine

Yield: 19.2 mg (68% of theory)

HPLC (Method 10): Rₜ=3 5 min;

LC-MS (Method 12): Rₜ=1.41 min; MS (ESIpos): m/z=957 (M+H)⁺

By subsequently detaching the BOC protecting group with trifluoroacetic acid, 19.3 mg (99% of theory) of the title compound were then obtained.

HPLC (Method 10): Rₜ=2.52 min;

LC-MS (Method 1): Rₜ=0.86 min; MS (ESIpos): m/z=857 (M+H)⁺

### Intermediate 58

### N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

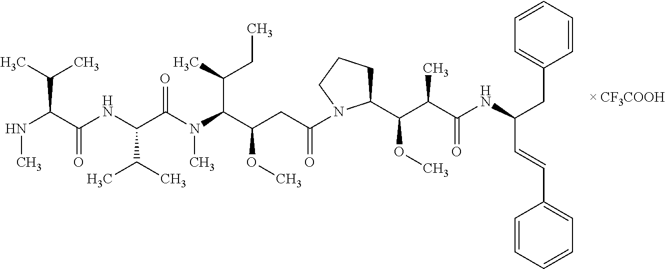 [SMILES: CC[C@H](C)[C@@H]([C@@H](CC(=O)N1CCC[C@H]1[C@H](OC)[C@@H](C)C(=O)N[C@H](/C=C/c1ccccc1)Cc1ccccc1)OC)N(C)C(=O)[C@@H](NC(=O)[C@@H](NC)C(C)C)C(C)C]

N-(tert-Butoxycarbonyl)-N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared in analogy to the synthesis of Intermediate 55, by reaction of 20 mg (29 μmol) N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide with 7.1 mg (32 μmol) of (2S,3E)-1,4-diphenylbut-3-en-2-amine

Yield: 15.1 mg (58% of theory)

HPLC (Method 10): Rₜ=4.2 min;

LC-MS (Method 12): Rₜ=1.51 min; MS (ESIpos): m/z=891 (M+H)

By subsequently detaching the BOC protecting group with trifluoroacetic acid, 15.7 mg (99% of theory) of the title compound were then obtained.

HPLC (Method 10): Rₜ=2.62 min;

LC-MS (Method 12): Rₜ=0.97 min; MS (ESIpos): m/z=791 (M+H)⁺

### Intermediate 61

### N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

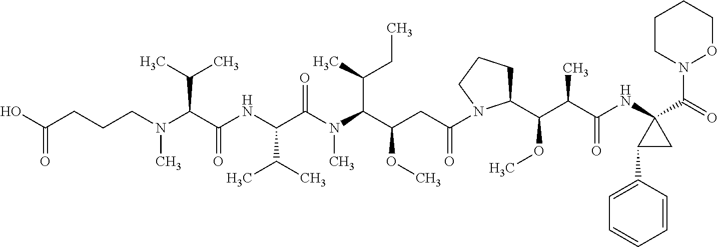 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)O)C(C)C]

50 mg (0.054 mmol) of N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide trifluoroacetate (Intermediate 16) were dissolved in 8 ml of dioxane/water, and 70 ml (0.108 mmol) of a 15% solution of 4-oxobutanoic acid in water were added. The reaction mixture was subsequently stirred at 100° C. for 1 h. After cooling to RT, 3.7 mg (0.059 mmol) of sodium cyanoborohydride were added and the mixture was adjusted to a pH of 3 by adding about 300 μl of 0.1 N hydrochloric acid. The reaction mixture was then stirred at 100° C. for a further 2 h. After cooling, another 70 ml (0.108 mmol) of the 15% 4-oxobutanoic acid solution were added and the reaction mixture was again stirred at 100° C. for 1 h. Then a further 3.7 mg (0.059 mmol) of sodium cyanoborohydride were added and about 300 μl of 0.1 N hydrochloric acid were subsequently used to adjust the pH back to 3. The reaction mixture was then stirred at 100° C. for another 2 h. In the event of conversion still being incomplete, this procedure was repeated for a third time. The reaction mixture was finally concentrated and the residue was purified by means of preparative HPLC. In this way, 32 mg (65% of theory) of the title compound were obtained in the form of a colourless foam.

HPLC (Method 5): Rₜ=1.64 min;

LC-MS (Method 9): Rₜ=4.76 min; MS (ESIpos): m/z=899 (M+H)⁺

¹H NMR (500 MHz, DMSO-d₆): δ=8.95 and 8.8 (2m, 1H), 8.88 and 8.65 (2s, 1H), 7.4-7.1 (m, 5H), 5.0, 4.78, 4.65 and 4.55 (4m, 2H), 4.1-3.7 (m, 5H), 3.32, 3.29, 3.20, 3.12, 3.1 and 3.0 (6s, 9H), 2.75 (m, 2H), 2.63 (t, 1H), 2.4-2.2 (m, 4H), 2.1-1.2 (m, 12H), 1.2-0.8 (m, 16H), 0.75 (m, 3H) [further signals hidden under H₂O and DMSO peaks].

### Intermediate 62

### N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

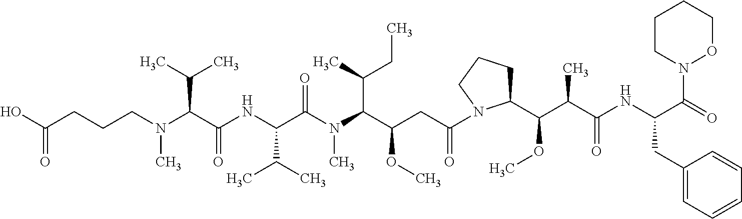 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCC(=O)O)C(C)C]

The title compound was prepared in analogy to the synthesis of Intermediate 61, by reaction of 50 mg of N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide trifluoroacetate (Intermediate 14) with 4-oxobutanoic acid.

Yield: 34 mg (70% of theory)

HPLC (Method 5): Rₜ=1.64 min;

LC-MS (Method 9): Rₜ=4.77 min; MS (ESIpos): m/z=887 (M+H)⁺.

### Intermediate 63

### N-(4-carboxybenzyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

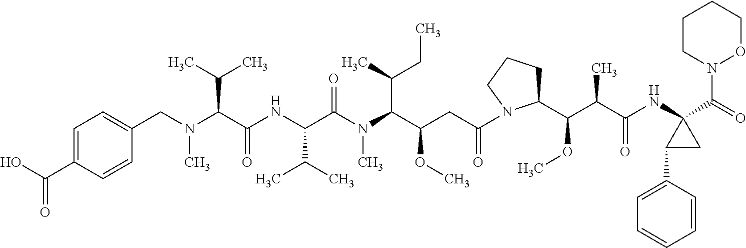 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)Cc1ccc(C(=O)O)cc1)C(C)C]

The title compound was prepared in analogy to the synthesis of Intermediate 61, by reaction of 15 mg of N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide trifluoroacetate (Intermediate 16) with 4-formylbenzoic acid.

Yield: 7.5 mg (48% of theory)

HPLC (Method 5): Rₜ=1.75 min;

LC-MS (Method 1): Rₜ=0.97 min; MS (ESIpos): m/z=947 (M+H)⁺.

### Intermediate 64

### N-(5-carboxypentyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

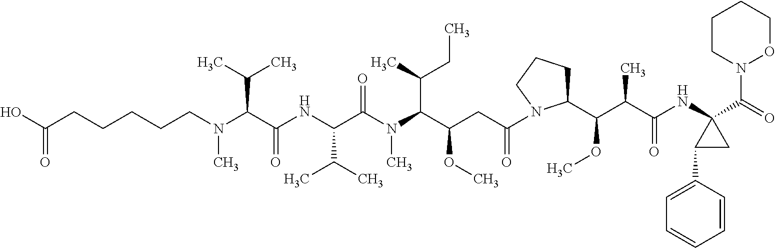 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCCCC(=O)O)C(C)C]

10 mg (0.011 mmol) of N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide trifluoroacetate (Intermediate 16) were dissolved in 2 ml of dioxane/water, and 2.8 mg (0.022 mmol) of 6-oxohexanoic acid were added. The reaction mixture was subsequently stirred at 100° C. for 1 h. After cooling to RT, 0.75 mg (0.012 mmol) of sodium cyanoborohydride was added and the mixture was adjusted to a pH of 3 by adding 0.1 N hydrochloric acid. The reaction mixture was then stirred at 100° C. for a further hour. After cooling, another 2.8 mg (0.022 mmol) of 6-oxohexanoic acid were added and the reaction mixture was again stirred at 100° C. for 1 h. A further 0.75 mg (0.012 mmol) of sodium cyanoborohydride was added and 0.1 N hydrochloric acid was subsequently used to adjust the pH back to 3. The reaction mixture was then stirred at 100° C. for another 1 h. This procedure was then repeated for a third time. The reaction mixture was finally concentrated and the crude product was purified by means of preparative HPLC. This gave 6.4 mg (64% of theory) of the title compound in the form of a colourless foam.

HPLC (Method 5): Rₜ=1.68 min;

LC-MS (Method 9): Rₜ=4.86 min; MS (ESIpos): m/z=927 (M+H)⁺.

### Intermediate 65

### N-(2-aminoethyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide bistrifluoroacetate

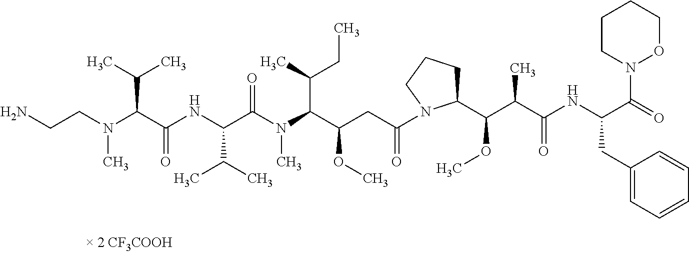 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCN)C(C)C]

The title compound was prepared by reaction of 68 mg of N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide trifluoroacetate (Intermediate 14) with tert-butyl 2-oxoethyl carbamate and subsequent detachment of the Boc protecting group with trifluoroacetic acid.

Yield: 49 mg (62% of theory over two stages)

HPLC (Method 5): Rₜ=1.58 min;

LC-MS (Method 2): Rₜ=1.05 min; MS (ESIpos): m/z=844 (M+H)⁺

¹H NMR (600 MHz, DMSO-d₆): δ=8.25 (m, 1H), 8.45 and 8.15 (2d, 1H), 7.65-7.55 (m, 3H), 7.23-7.1 (m, 5H), 5.12 and 4.95 (2m, 1H), 4.72 and 4.62 (2m, 1H), 4.6 and 4.52 (2t, 1H), 4.2-3.8 (m, 4H), 3.7 (d, 1H), 3.23, 3.20, 3.19, 3.18, 3.03 and 2.98 (6s, 9H), 3.0-2.7 (m, 6H), 2.4-1.2 (m, 15H), 1.05, 1.0, 0.88 and 0.82 (4d, 6H), 0.92 (m, 6H), 0.73 (m, 6H) [further signals hidden under H₂O peak].

### Intermediate 66

### N-(3-aminopropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

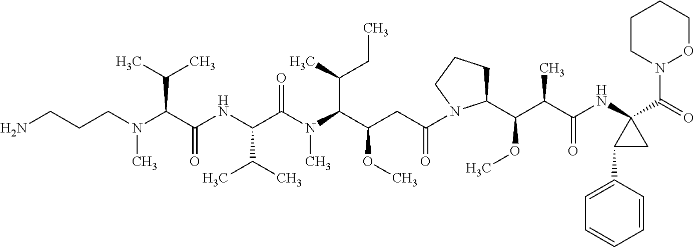 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCN)C(C)C]

The title compound was prepared in analogy to the synthesis of Intermediate 65, by reaction of 25 mg (0.027 mmol) of N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide trifluoroacetate (Intermediate 16) with benzyl 3-oxopropyl carbamate and subsequent hydrogenolytic detachment of the Z protecting group (with 10% palladium on charcoal as a catalyst, in ethanol as a solvent).

Yield: 11 mg (41% of theory over two stages)

HPLC (Method 5): Rₜ=1.53 min;

LC-MS (Method 1): Rₜ=0.72 min; MS (ESIpos): m/z=870 (M+H)⁺.

### Intermediate 67

### N-(3-carboxypropyl)-N-methyl-L-valyl-N-[(3R,4S,5S)-1-{(2S)-2-[(1R,2R)-3-{[(2S)-1-(adamantan-1-ylmethoxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

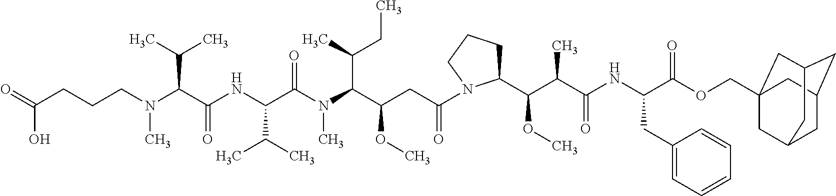 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCC12CC3CC(CC(C3)C1)C2)OC)N(C)C(=O)[C@@H](NC(=O)[C@H](C(C)C)N(C)CCCC(=O)O)C(C)C]

26 mg (26 μmol) of N-methyl-L-valyl-N-[(3R,4S,5S)-1-{(2S)-2-[(1R,2R)-3-{[(2S)-1-(adamantan-1-ylmethoxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate and 33.9 μl of a 15% aqueous succinaldehydic acid solution (53 μmol) were dissolved in 957 μl of a 1:1-dioxane/water mixture and heated to 100° C. for 1 h. After brief cooling, 1.81 mg (29 μmol) of sodium cyanoborohydride were added. The reaction mixture was adjusted to pH 3 by adding 0.1 N hydrochloric acid and the mixture was heated to 100° C. for a further 2 h. After again adding the same amounts of succinaldehydic acid solution, sodium cyanoborohydride and hydrochloric acid, the mixture was heated once again to 100° C. for 2 h. The reaction mixture was then separated directly into its components by means of preparative HPLC. 18.5 mg (73% of theory) of the title compound were thus obtained.

LC-MS (Method 1): Rₜ=1.17 min; m/z=967 (M+H)⁺.

### Intermediate 68

### N-(3-carboxypropyl)-N-methyl-L-valyl-N-[(3R,4S,5S)-1-{(2S)-2-[(1R,2R)-3-{[(2S)-1-(benzyloxy)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

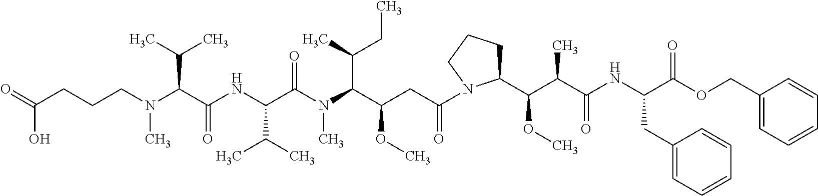 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccccc1)OC)N(C)C(=O)[C@@H](NC(=O)[C@H](C(C)C)N(C)CCCC(=O)O)C(C)C]

24 mg (26 μmol) of N-methyl-L-valyl-N-[(3R,4S,5S)-1-{(2S)-2-[(1R,2R)-3-{[(2S)-1-(benzyloxy)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate and 33.7 μl of a 15% aqueous succinaldehydic acid solution (52 μmol) were dissolved in 953 μl of a 1:1-dioxane/water mixture and heated to 100° C. for 1 h. After brief cooling, 1.80 mg (29 μmol) of sodium cyanoborohydride were added. The reaction mixture was adjusted to pH 3 by adding 0.1 N hydrochloric acid and the mixture was heated to 100° C. for a further 2 h. After again adding the same amounts of succinaldehydic acid solution, sodium cyanoborohydride and hydrochloric acid, the mixture was heated once again to 100° C. for 2 h. The reaction mixture was then separated directly into its components by means of preparative HPLC. 15.2 mg (65% of theory) of the title compound were thus obtained.

LC-MS (Method 1): Rₜ=1.01 min; m/z=895 (M+H)⁺

### Intermediate 69

### N-(3-carboxypropyl)-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

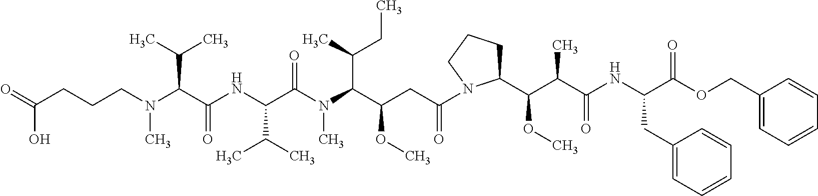 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccccc1)OC)N(C)C(=O)[C@@H](NC(=O)[C@H](C(C)C)N(C)CCCC(=O)O)C(C)C]

53 mg (84 μmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) and 45 mg (84 μmol) of benzyl N-{(2R,3R)-3-methoxy-2-methyl-3-[(2S)-pyrrolidin-2-yl]propanoyl}-L-phenylalaninate trifluoroacetate (Intermediate 12) were taken up in 2 ml of DMF, 19 μl of N,N-diisopropylethylamine, 14 mg (92 μmol) of HOBt and 17.6 mg (92 μmol) of EDC were added and then the mixture was stirred at RT overnight. Subsequently, the reaction mixture was concentrated and the residue was purified by means of preparative HPLC. This gave 59 mg (68% of theory) of the Fmoc-protected intermediate N-[(9H-fluoren-9-ylmethoxy)carbonyl]-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide.

LC-MS (Method 1): Rₜ=1.55 min; m/z=1044 (M+H)⁺.

57 mg (0.055 mmol) of this intermediate were treated with 1.2 ml of piperidine in 5 ml of DMF to detach the Fmoc protecting group. After concentration and purification by means of preparative HPLC, 39 mg (76% of theory) of the free amine intermediate N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were obtained as the trifluoroacetate.

HPLC (Method 5): Rₜ=1.9 min;

LC-MS (Method 1): Rₜ=1.01 min; m/z=822 (M+H)⁺.

37 mg (0.045 mmol) of this intermediate were dissolved in 5 ml of dioxane/water and, analogously to the preparation of the compound in Intermediate 66, reacted with a 15% aqueous solution of 4-oxobutanoic acid in the presence of sodium cyanoborohydride. 16 mg (39% of theory) of the title compound were obtained in the form of a colourless foam.

HPLC (Method 6): Rₜ=2.1 min;

LC-MS (Method 1): Rₜ=1.01 min; MS (ESIpos): m/z=908 (M+H)⁺.

### Intermediate 70

### N-(3-carboxypropyl)-N-methyl-L-valyl-N-[(3R,4S,5S)-1-{(2S)-2-[(1R,2R)-3-{[(2S,3S)-1-(benzyloxy)-1-oxo-3-phenylbutan-2-yl]amino}-1-methoxy-2-methyl-3-oxopropyl]pyrrolidin-1-yl}-3-methoxy-5-methyl-1-oxoheptan-4-yl]-N-methyl-L-valinamide

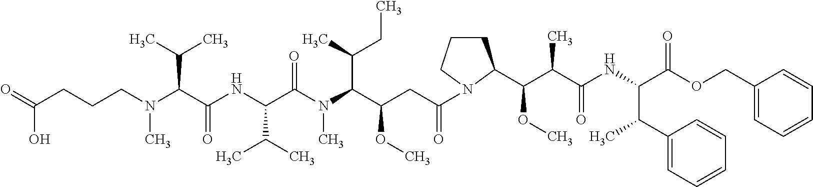 [SMILES: CC[C@H](C)[C@@H]([C@@H](CC(=O)N1CCC[C@H]1[C@H](OC)[C@@H](C)C(=O)N[C@H](C(=O)OCc1ccccc1)[C@@H](C)c1ccccc1)OC)N(C)C(=O)[C@@H](NC(=O)[C@H](C(C)C)N(C)CCCC(=O)O)C(C)C]

First, in analogy to the synthesis described in Intermediate 14, proceeding from Intermediates 4 and 26, the amine compound N-methyl-L-valyl-N-[(3R,4S,5S)-1-{(2S)-2-[(1R,2R)-3-{[(2S,3S)-1-(benzyloxy)-1-oxo-3-phenylbutan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared.

30 mg (0.032 mmol) of this compound were dissolved in 6 ml of dioxane/water, and 41 μl (0.063 mmol) of a 15% aqueous solution of 4-oxobutanoic acid were added. The reaction mixture was subsequently stirred at 100° C. for 1 h. After cooling to RT, 2.2 mg (0.035 mmol) of sodium cyanoborohydride were added and the mixture was adjusted to a pH of 3 by adding about 300 μl of 0.1 N hydrochloric acid. The reaction mixture was then stirred at 100° C. for a further 2 h. After cooling, another 41 μl (0.063 mmol) of the 15% 4-oxobutanoic acid solution were added and the reaction mixture was again stirred at 100° C. for 1 h. Then a further 2.2 mg (0.035 mmol) of sodium cyanoborohydride were added and about 300 μl of 0.1 N hydrochloric acid were subsequently used to adjust the pH back to 3. The reaction mixture was then stirred at 100° C. for another 2 h. In the event of conversion still being incomplete, this procedure was repeated for a third time. The reaction mixture was finally concentrated and the crude product was purified by means of preparative HPLC. This gave 24 mg (82% of theory) of the title compound in the form of a colourless foam.

HPLC (Method 5): Rₜ=1.9 min;

LC-MS (Method 9): Rₜ=5.15 min; MS (ESIpos): m/z=922 (M+H)⁺.

### Intermediate 71

### N-(3-carboxypropyl)-N-methyl-L-valyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl-1-oxoheptan-4-yl]-N-methyl-L-valinamide

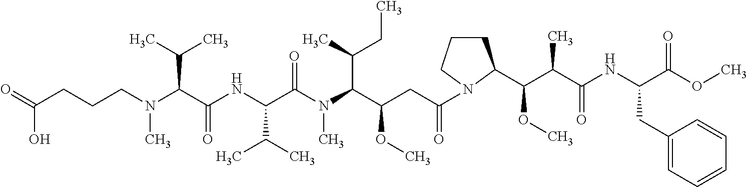 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)CCCC(=O)O)C(C)C]

First, in analogy to the synthesis described in Intermediate 14, proceeding from Intermediates 4 and 39, the amine compound N-methyl-L-valyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl-1-oxoheptan-4-yl]-N-methyl-L-valinamide was prepared. 7 mg (0.009 mmol) of this compound were then used, in analogy to the preparation of Intermediate 61, by reaction with 4-oxobutanoic acid in the presence of sodium cyanoborohydride, to obtain 2 mg (22% of theory) of the title compound in the form of a colourless foam.

HPLC (Method 6): Rₜ=1.9 min;

LC-MS (Method 2): Rₜ=1.06 min; MS (ESIpos): m/z=832 (M+H)⁺.

### Intermediate 72

### N-(3-carboxypropyl)-N-methyl-L-valyl-N-[(3R,4S,5S)-1-{(2S)-2-[(1R,2R)-3-{[(2S)-1-(benzyloxy)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

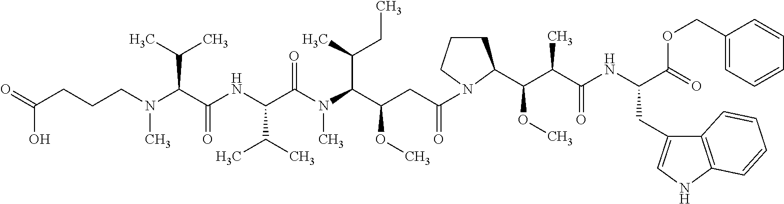 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)OCc1ccccc1)OC)N(C)C(=O)[C@@H](NC(=O)[C@H](C(C)C)N(C)CCCC(=O)O)C(C)C]

212 mg (411 μmol) of N-(tert-butoxycarbonyl)-N-methyl-L-valyl-N-[(2R,3S,4S)-1-carboxy-2-methoxy-4-methylhexan-3-yl]-N-methyl-L-valinamide (Intermediate 8) and 237 mg (411 μmol) of benzyl-N-{(2R,3R)-3-methoxy-2-methyl-3-[(2S)-pyrrolidin-2-yl]propanoyl}-L-tryptophanate trifluoroacetate (Intermediate 20) were taken up in 30 ml of DMF, and 188 mg (493 μmol) of O-(7-azabenzotriazol-1-yl)-N,N,N′,N′-tetramethyluronium hexafluorophosphate and 215 μl N,N-diisopropylethylamine were added. The reaction mixture was stirred at RT for 20 h, then concentrated under reduced pressure, and the residue was purified by means of preparative HPLC. The product fractions were combined and concentrated, and the residue was dried under high vacuum. This gave 315 mg (80% of theory) of the Boc-protected intermediate N-(tert-butoxycarbonyl)-N-methyl-L-valyl-N-[(3R,4S,5S)-1-{(2S)-2-[(1R,2R)-3-{[(2S)-1-(benzyloxy)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide as a colourless foam.

LC-MS (Method 1): Rₜ=1.45 min; m/z=961 (M+H)⁺.

50 mg (52 μmol) of this intermediate were treated with 1 ml of trifluoroacetic acid in 9 ml of dichloromethane to detach the Boc protecting group. After concentration and purification by means of preparative HPLC, 29 mg (57% of theory) of the free amine intermediate N-methyl-L-valyl-N-[(3R,4S,5S)-1-{(2S)-2-[(1R,2R)-3-{[(2S)-1-(benzyloxy)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide were obtained as the trifluoroacetate.

LC-MS (Method 1): Rₜ=0.99 min; m/z=861 (M+H)⁺.

29 mg (0.03 mmol) of this intermediate were dissolved in 6 ml of dioxane/water, and 39 μl (0.059 mmol) of a 15% aqueous solution of 4-oxobutanoic acid were added. The reaction mixture was subsequently stirred at 100° C. for 1 h. After cooling to RT, 2 mg (0.033 mmol) of sodium cyanoborohydride were added and the mixture was adjusted to a pH of 3 by adding about 300 μl of 0.1 N hydrochloric acid. The reaction mixture was then stirred at 100° C. for a further 2 h. After cooling, another 39 μl (0.059 mmol) of the 15% 4-oxobutanoic acid solution were added and the reaction mixture was again stirred at 100° C. for 1 h. Then a further 2 mg (0.033 mmol) of sodium cyanoborohydride were added and about 300 μl of 0.1 N hydrochloric acid were subsequently used to adjust the pH back to 3. The mixture was then stirred at 100° C. for another 2 h. Thereafter, the reaction mixture was poured onto a 1:1 mixture of semisaturated aqueous ammonium chloride solution and ethyl acetate. The organic phase was removed, washed with saturated sodium chloride solution, dried over sodium sulphate and concentrated. The residue was freeze-dried from water/acetonitrile. This gave 27 mg (94% of theory) of the title compound in the form of a colourless foam.

HPLC (Method 5): Rₜ=2.2 min;

LC-MS (Method 9): Rₜ=5.04 min; MS (ESIpos): m/z=947 (M+H)⁺.

### Intermediate 73

### N-(3-carboxypropyl)-N-methyl-L-valyl-N-[(3R,4S,5S)-1-{(2S)-2-[(1R,2R)-3-({(2S)-1-[benzyl(methyl)amino]-1-oxo-3-phenylpropan-2-yl}amino)-1-methoxy-2-methyl-3-oxopropyl]pyrrolidin-1-yl}-3-methoxy-5-methyl-1-oxoheptan-4-yl]-N-methyl-L-valinamide

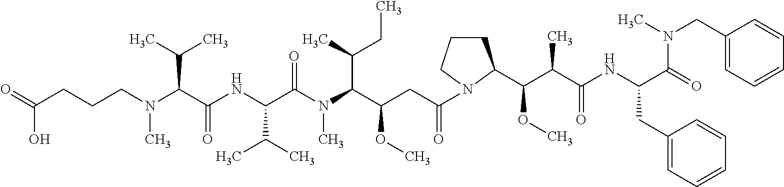 [SMILES: CC[C@H](C)[C@@H]([C@@H](CC(=O)N1CCC[C@H]1[C@H](OC)[C@@H](C)C(=O)N[C@@H](Cc1ccccc1)C(=O)N(C)Cc1ccccc1)OC)N(C)C(=O)[C@@H](NC(=O)[C@H](C(C)C)N(C)CCCC(=O)O)C(C)C]

First, in analogy to the synthesis described in Intermediate 14, proceeding from Intermediates 4 and 38, the amine compound N-methyl-L-valyl-N-[(3R,4S,5S)-1-{(2S)-2-[(1R,2R)-3-({(2S)-1-[benzyl(methyl)amino]-1-oxo-3-phenylpropan-2-yl}amino)-1-methoxy-2-methyl-3-oxopropyl]pyrrolidin-1-yl}-3-methoxy-5-methyl-1-oxoheptan-4-yl]-N-methyl-L-valinamide was prepared. 25 mg (0.026 mmol) of this compound were then used, in analogy to the preparation of Intermediate 61, by reaction with 4-oxobutanoic acid in the presence of sodium cyanoborohydride, to obtain 13 mg (54% of theory) of the title compound in the form of a colourless foam.

HPLC (Method 12): Rₜ=2.2 min;

LC-MS (Method 9): Rₜ=5.01 min; MS (ESIpos): m/z=921 (M+H)⁺.

### Intermediate 74

### N-(3-carboxypropyl)-N-methyl-L-valyl-N-[(3R,4S,5S)-1-{(2S)-2-[(1R,2R)-3-({(1S,2R)-1-[(benzyloxy)carbonyl]-2-phenylcyclopropyl}amino)-1-methoxy-2-methyl-3-oxopropyl]pyrrolidin-1-yl}-3-methoxy-5-methyl-1-oxoheptan-4-yl]-N-methyl-L-valinamide

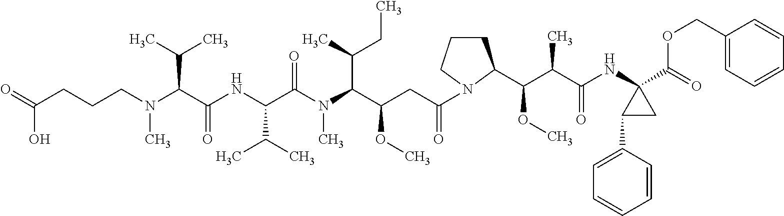 [SMILES: CC[C@H](C)[C@@H]([C@@H](CC(=O)N1CCC[C@H]1[C@H](OC)[C@@H](C)C(=O)N[C@@]1(C(=O)OCc2ccccc2)C[C@@H]1c1ccccc1)OC)N(C)C(=O)[C@@H](NC(=O)[C@H](C(C)C)N(C)CCCC(=O)O)C(C)C]

50 mg (73 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and 28 mg (73 μmol) of benzyl (1S,2R)-1-amino-2-phenylcyclopropanecarboxylate trifluoroacetate (Intermediate 45) were taken up in 5 ml of DMF, and 42 mg (110 μmol) of O-(7-azabenzotriazol-1-yl)-N,N,N′,N′-tetramethyluronium hexafluorophosphate and 38 μl of N,N-diisopropylethylamine were added. The reaction mixture was stirred at RT for 5 h, then concentrated under reduced pressure, and the residue was purified by means of preparative HPLC. The product fractions were combined and concentrated. After lyophilization from dioxane/water, 35 mg (51% of theory) of the Boc-protected intermediate N-(tert-butoxycarbonyl)-N-methyl-L-valyl-N-[(3R,4S,5S)-1-{(2S)-2-[(1R,2R)-3-({(1S,2R)-1-[(benzyloxy)carbonyl]-2-phenylcyclopropyl}amino)-1-methoxy-2-methyl-3-oxopropyl]pyrrolidin-1-yl}-3-methoxy-5-methyl-1-oxoheptan-4-yl]-N-methyl-L-valinamide were obtained as a colourless foam.

LC-MS (Method 1): Rₜ=1.52 min; m/z=934 (M+H)⁺.

35 mg of this intermediate were treated with 1 ml of trifluoroacetic acid in 5 ml of dichloromethane to detach the Boc protecting group. After concentration and lyophilization from dioxane/water, 34 mg (97% of theory) of the free amine intermediate N-methyl-L-valyl-N-[(3R,4S,5S)-1-{(2S)-2-[(1R,2R)-3-({(1S,2R)-1-[(benzyloxy)carbonyl]-2-phenylcyclopropyl}amino)-1-methoxy-2-methyl-3-oxopropyl]pyrrolidin-1-yl}-3-methoxy-5-methyl-1-oxoheptan-4-yl]-N-methyl-L-valinamide were obtained as the trifluoroacetate.

LC-MS (Method 1): Rₜ=0.91 min; m/z=834 (M+H)⁺.

11 mg (0.011 mmol) of this intermediate were then used, in analogy to the preparation of Intermediate 66, by reaction with 4-oxobutanoic acid in the presence of sodium cyanoborohydride, to obtain 2.5 mg (24% of theory) of the title compound in the form of a colourless foam.

HPLC (Method 12): Rₜ=2.2 min;

LC-MS (Method 9): Rₜ=5.1 min; MS (ESIpos): m/z=920 (M+H)⁺.

### Intermediate 75

### N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(1S,2R)-2-phenyl-1-(propylcarbamoyl)cyclopropyl]amino}propyl]pyrrolidin-1-yl}-5-methyl-1-oxoheptan-4-yl]-N-methyl-L-valinamide

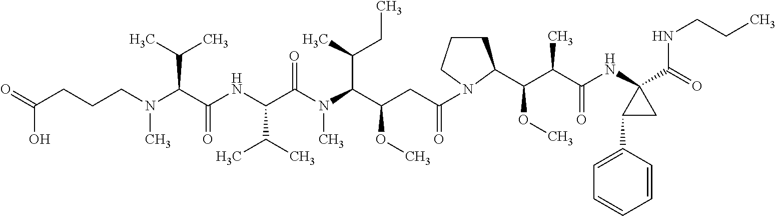 [SMILES: CCCNC(=O)[C@]1(NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)CCCC(=O)O)C(C)C)C[C@@H]1c1ccccc1]

First, in analogy to the synthesis described in Intermediate 74, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and (1S,2R)-1-amino-2-phenyl-N-propylcyclopropanecarboxamide trifluoroacetate (Intermediate 27) in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-[(3R,4S,5S)-3-methoxy-1-{(2S)-2-[(1R,2R)-1-methoxy-2-methyl-3-oxo-3-{[(1S,2R)-2-phenyl-1-(propylcarbamoyl)cyclopropyl]amino}propyl]pyrrolidin-1-yl}-5-methyl-1-oxoheptan-4-yl]-N-methyl-L-valinamide was prepared as the trifluoroacetate. 14 mg (0.016 mmol) of this compound were then used, in analogy to the preparation of Intermediate 61, by reaction with 4-oxobutanoic acid in the presence of sodium cyanoborohydride, to obtain 11.3 mg (83% of theory) of the title compound.

HPLC (Method 6): Rₜ=1.9 min;

LC-MS (Method 2): Rₜ=1.27 min; MS (ESIpos): m/z=871 (M+H)⁺.

### Intermediate 76

### N-(3-carboxypropyl)-N-methyl-L-valyl-N-[(3R,4S,5S)-1-{(2S)-2-[(1R,2R)-3-{[(1S,2R)-1-(ethoxycarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

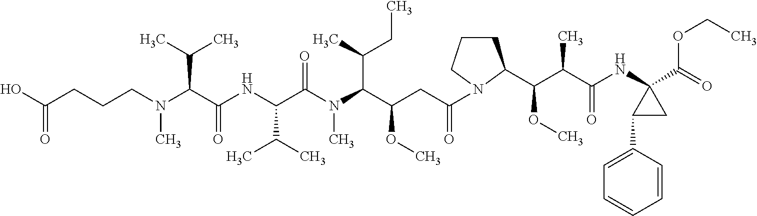 [SMILES: CCOC(=O)[C@]1(NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)CCCC(=O)O)C(C)C)C[C@@H]1c1ccccc1]

First, by coupling of Intermediate 46 (N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide) with Intermediate 48 (ethyl(1S,2R)-1-amino-2-phenylcyclopropanecarboxylate trifluoroacetate) in the presence of O-(7-azabenzotriazol-1-yl)-N,N,N′,N′-tetramethyluronium hexafluorophosphate and subsequent Boc detachment, the starting compound N-methyl-L-valyl-N-[(3R,4S,5S)-1-{(2S)-2-[(1R,2R)-3-{[(1S,2R)-1-(ethoxycarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate was prepared. 70 mg (0.079 mmol) of this starting material were then used, by reaction with 4-oxobutanoic acid, in analogy to Intermediate 61, to obtain 46 mg (68% of theory) of the title compound.

HPLC (Method 6): Rₜ=1.9 min;

LC-MS (Method 2): Rₜ=1.28 min; MS (ESIpos): m/z=858 (M+H)⁺

### Intermediate 77

### N-(3-carboxypropyl)-N-methyl-L-valyl-N-[(3R,4S,5S)-1-{(2S)-2-[(1R,2R)-3-{[(2S)-1-amino-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

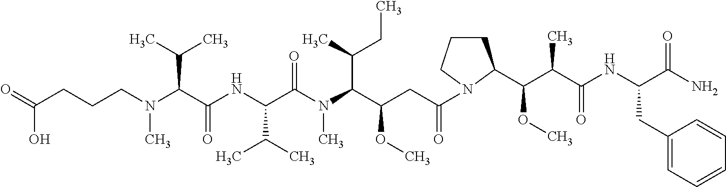 [SMILES: CC[C@H](C)[C@@H]([C@@H](CC(=O)N1CCC[C@H]1[C@H](OC)[C@@H](C)C(=O)N[C@@H](Cc1ccccc1)C(N)=O)OC)N(C)C(=O)[C@@H](NC(=O)[C@H](C(C)C)N(C)CCCC(=O)O)C(C)C]

First, in analogy to the synthesis described in Intermediate 75, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and L-phenylalaninamide hydrochloride in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-[(3R,4S,5S)-1-{(2S)-2-[(1R,2R)-3-{[(2S)-1-amino-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 47 mg (0.049 mmol) of this compound were then used, in analogy to the preparation of Intermediate 61, by reaction with 4-oxobutanoic acid in the presence of sodium cyanoborohydride, to obtain 39 mg (96% of theory) of the title compound.

HPLC (Method 6): Rₜ=1.7 min;

LC-MS (Method 9): Rₜ=4.44 min; MS (ESIpos): m/z=817 (M+H)⁺

¹H NMR (500 MHz, DMSO-d₆): δ=8.95 and 8.8 (2m, 1H), 8.25 and 8.0 (2d, 1H), 7.45, 7.35 and 7.0 (3s, broad, 2H), 7.3-7.1 (m, 5H), 4.8-4.4 (2m, 3H), 3.95 (m, 1H), 3.82 (m, 1H), 3.72 (d, 1H), 3.22, 3.18, 3.15, 3.05 and 3.00 (5s, 9H), 2.85-2.7 (m, 4H), 2.45-1.6 (m, 12H), 1.5-1.2 (m, 3H), 1.1-0.7 (m, 21H) [further signals hidden under solvent peaks].

### Intermediate 78

### N-(6-aminohexyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

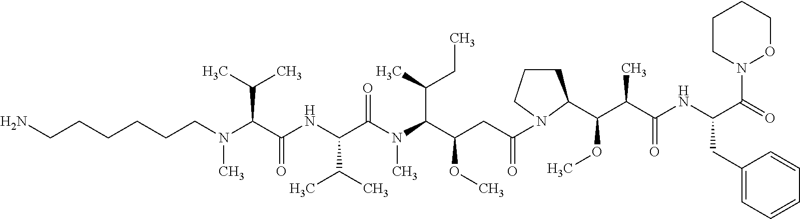 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCCCCN)C(C)C]

This compound was prepared in analogy to Intermediate 66 over 2 stages, proceeding from 20 mg (16 μmol) of the compound from Intermediate 14 and benzyl 6-oxohexyl carbamate, and the hydrogenation was performed in methanol as the solvent.

Yield: 7.6 mg (55% of theory over 2 stages)

HPLC (Method 6): Rₜ=1.8 min;

LC-MS (Method 1): Rₜ=0.7 min; MS (ESIpos): m/z=901 (M+H)⁺.

### Intermediate 79

### N-(3-carboxypropyl)-N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

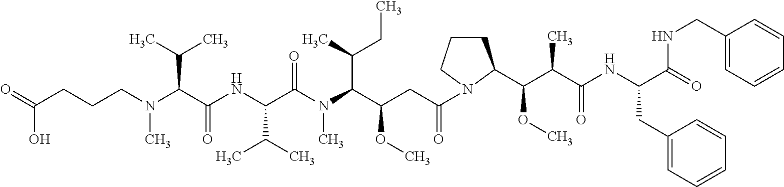 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCc1ccccc1)OC)N(C)C(=O)[C@@H](NC(=O)[C@H](C(C)C)N(C)CCCC(=O)O)C(C)C]

36 mg (43 μmol) of N-(tert-butoxycarbonyl)-N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide (Intermediate 46) and 4.6 mg (43 μmol) of benzylamine were taken up in 5 ml of DMF, 7.5 μl (88 μmol) of N,N-diisopropylethylamine, 10 mg (65 μmol) of HOBt and 10 mg (52 μmol) of EDC were added, and then the mixture was stirred at RT overnight. Subsequently, the reaction mixture was concentrated and the residue was purified by means of preparative HPLC. 29 mg (73% of theory) of the Boc-protected intermediate N-(tert-butoxycarbonyl)-N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were obtained.

LC-MS (Method 1): Rₜ=1.43 min; m/z=921 (M+H)⁺.

29 mg of this intermediate were treated with 1 ml of trifluoroacetic acid in 6 ml of dichloromethane to detach the Boc protecting group. After concentration and lyophilization from dioxane/water, 30 mg (quant.) of the free amine intermediate N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were obtained as the trifluoroacetate.

LC-MS (Method 1): Rₜ=0.95 min; m/z=821 (M+H)⁺.

17 mg (0.018 mmol) of this intermediate were then used, in analogy to the preparation of Intermediate 61, by reaction with 4-oxobutanoic acid in the presence of sodium cyanoborohydride, to obtain 13 mg (80% of theory) of the title compound in the form of a colourless foam.

HPLC (Method 5): Rₜ=1.7 min;

LC-MS (Method 9): Rₜ=4.97 min; MS (ESIpos): m/z=907 (M+H)⁺.

### Intermediate 80

### N-(3-carboxypropyl)-N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

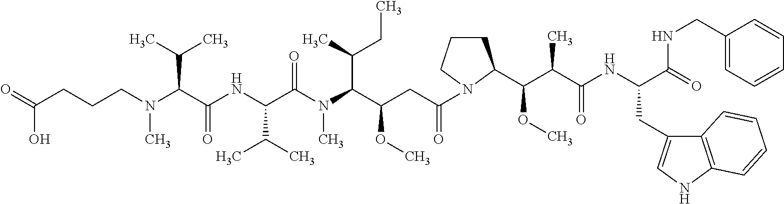 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)NCc1ccccc1)OC)N(C)C(=O)[C@@H](NC(=O)[C@H](C(C)C)N(C)CCCC(=O)O)C(C)C]

First, in analogy to the synthesis described in Intermediate 74, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and N-benzyl-L-tryptophanamide trifluoroacetate (Intermediate 47) in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 10 mg (0.01 mmol) of this compound were then used, in analogy to the preparation of Intermediate 61, by reaction with 4-oxobutanoic acid in the presence of sodium cyanoborohydride, to obtain 2.5 mg (26% of theory) of the title compound.

HPLC (Method 5): Rₜ=1.7 min;

LC-MS (Method 2): Rₜ=1.13 min; MS (ESIpos): m/z=946 (M+H)⁺.

### Intermediate 81

### N-(3-carboxypropyl)-N-methyl-L-valyl-N-[(3R,4S,5S)-1-{(2S)-2-[(1R,2R)-3-{[(1S,2R)-1-carbamoyl-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

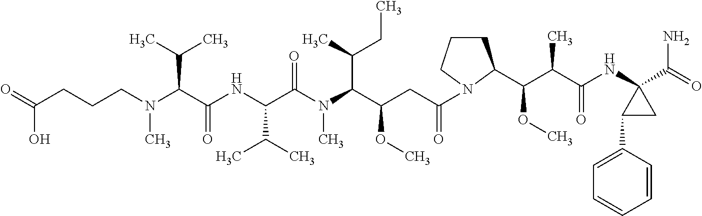 [SMILES: CC[C@H](C)[C@@H]([C@@H](CC(=O)N1CCC[C@H]1[C@H](OC)[C@@H](C)C(=O)N[C@@]1(C(N)=O)C[C@@H]1c1ccccc1)OC)N(C)C(=O)[C@@H](NC(=O)[C@H](C(C)C)N(C)CCCC(=O)O)C(C)C]

First, in analogy to the synthesis described in Intermediate 74, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and (1S,2R)-1-amino-2-phenylcyclopropanecarboxamide trifluoroacetate (Intermediate 48) in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-[(3R,4S,5S)-1-{(2S)-2-[(1R,2R)-3-{[(1S,2R)-1-carbamoyl-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 14 mg (0.0163 mmol) of this compound were then used, in analogy to the preparation of Intermediate 61, by reaction with 4-oxobutanoic acid in the presence of sodium cyanoborohydride, to obtain 8 mg (57% of theory) of the title compound.

HPLC (Method 5): Rₜ=1.6 min;

LC-MS (Method 9): Rₜ=4.64 min; MS (ESIpos): m/z=829 (M+H)⁺.

### Intermediate 82

### N-(3-carboxypropyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

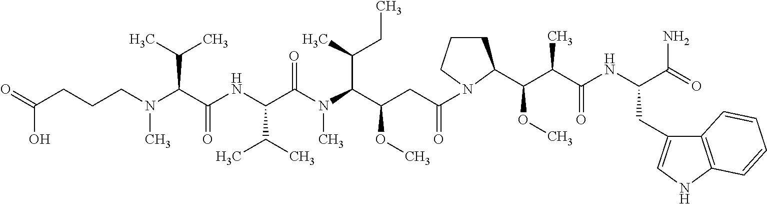 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)O)C(C)C]

First, in analogy to the synthesis described in Intermediate 69, by coupling of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) and N^{α}-{(2R,3R)-3-methoxy-2-methyl-3-[(2S)-pyrrolidin-2-yl]propanoyl}-L-tryptophanamide trifluoroacetate (Intermediate 49) in the presence of O-(7-azabenzotriazol-1-yl)-N,N,N′,N′-tetramethyluronium hexafluorophosphate and subsequent detachment of the Fmoc protecting group by means of piperidine, the amine compound N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 78 mg (0.088 mmol) of this compound were then used, in analogy to the preparation of Intermediate 61, by reaction with 4-oxobutanoic acid in the presence of sodium cyanoborohydride, to obtain 68 mg (90% of theory) of the title compound.

HPLC (Method 5): Rₜ=1.8 min;

LC-MS (Method 9): Rₜ=4.49 min; MS (ESIpos): m/z=856 (M+H)⁺.

### Intermediate 83

### N-(5-carboxypentyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

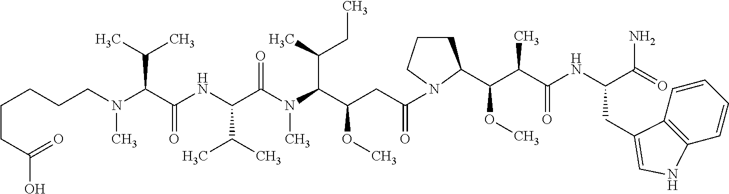 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCCCC(=O)O)C(C)C]

This compound was prepared in analogy to the compound in Intermediate 82, proceeding from 20 mg (26 μmol) of N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate, by reaction with 4-oxobutanoic acid in the presence of sodium cyanoborohydride prepared.

Yield: 5 mg (25% of theory)

HPLC (Method 5): Rₜ=1.6 min;

LC-MS (Method 11): Rₜ=0.72 min; MS (ESIpos): m/z=884 (M+H)⁺.

### Intermediate 84

### N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(morpholin-4-yl)-1-oxo-3-phenylpropan-2-yl]amino}-3-oxopropyl]pyrrolidin-1-yl}-5-methyl-1-oxoheptan-4-yl]-N-methyl-L-valinamide

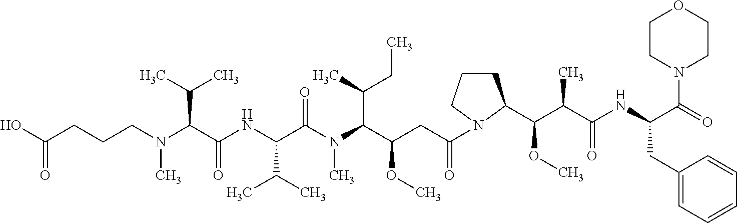

First, in analogy to the synthesis described in Intermediate 79, by coupling of N-(tert-butoxycarbonyl)-N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide (Intermediate 46) and morpholine in the presence of EDC and HOBT and subsequent detachment of the Boc protecting group by means of trifluoroacetic acid, the amine compound N-methyl-L-valyl-N-[(3R,4S,5S)-3-methoxy-1-{(2S)-2-[(1R,2R)-1-methoxy-2-methyl-3-{[(2S)-1-(morpholin-4-yl)-1-oxo-3-phenylpropan-2-yl]amino}-3-oxopropyl]pyrrolidin-1-yl}-5-methyl-1-oxoheptan-4-yl]-N-methyl-L-valinamide was prepared as the trifluoroacetate. 30 mg (0.033 mmol) of this compound were then used, in analogy to the preparation of Intermediate 61, by reaction with 4-oxobutanoic acid in the presence of sodium cyanoborohydride, to obtain 22 mg (76% of theory) of the title compound.

HPLC (Method 5): Rₜ=1.6 min;

LC-MS (Method 9): Rₜ=4.58 min; MS (ESIpos): m/z=887 (M+H)⁺.

### Intermediate 85

### N-(3-carboxypropyl)-N-methyl-L-valyl-N-[(3R,4S,5S)-1-{(2S)-2-[(1R,2R)-3-{[(2S,3R)-1-(benzylamino)-3-hydroxy-1-oxobutan-2-yl]amino}-1-methoxy-2-methyl-3-oxopropyl]pyrrolidin-1-yl}-3-methoxy-5-methyl-1-oxoheptan-4-yl]-N-methyl-L-valinamide

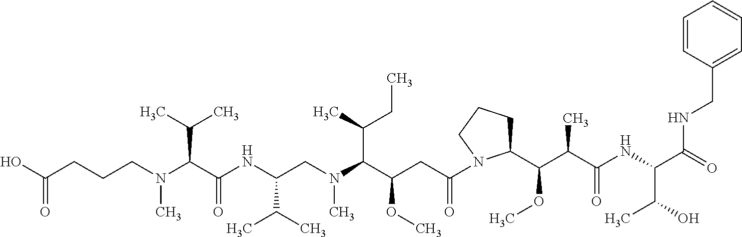 [SMILES: CC[C@H](C)[C@@H]([C@@H](CC(=O)N1CCC[C@H]1[C@H](OC)[C@@H](C)C(=O)N[C@H](C(=O)NCc1ccccc1)[C@@H](C)O)OC)N(C)C[C@@H](NC(=O)[C@H](C(C)C)N(C)CCCC(=O)O)C(C)C]

First, in analogy to the synthesis described in Intermediate 79, by coupling of N-(tert-butoxycarbonyl)-N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide (Intermediate 46) and N-benzyl-L-threoninamide trifluoroacetate in the presence of HATU and subsequent detachment of the Boc protecting group by means of trifluoroacetic acid, the amine compound N-methyl-L-valyl-N-[(3R,4S,5S)-1-{(2S)-2-[(1R,2R)-3-{[(2S,3R)-1-(benzylamino)-3-hydroxy-1-oxobutan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 21 mg (0.024 mmol) of this compound were then used, in analogy to the preparation of Intermediate 61, by reaction with 4-oxobutanoic acid in the presence of sodium cyanoborohydride, to obtain 20 mg (97% of theory) of the title compound.

HPLC (Method 5): Rₜ=1.54 min;

LC-MS (Method 9): Rₜ=4.49 min; MS (ESIpos): m/z=861 (M+H)⁺.

### Intermediate 86

### 4-{[(2S)-1-{[(2S)-1-{[(3R,4S,5S)-1-{(2S)-2-[(1R,2R)-3-{[(2S)-1-tert.-Butoxy-1-oxo-3-phenylpropan-2-yl]amino}-1-methoxy-2-methyl-3-oxopropyl]pyrrolidin-1-yl}-3-methoxy-5-methyl-1-oxoheptan-4-yl](methyl)amino}-3-methylbutan-2-yl]amino}-3-methyl-1-oxobutan-2-yl](methyl)amino}butanoic acid

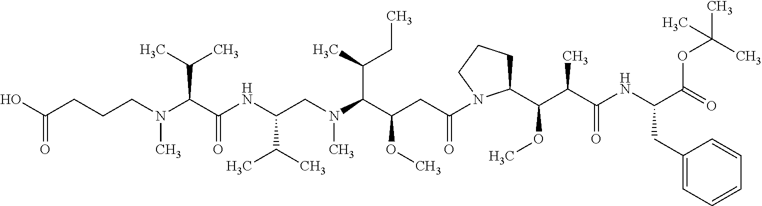 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC(C)(C)C)OC)N(C)C[C@@H](NC(=O)[C@H](C(C)C)N(C)CCCC(=O)O)C(C)C]

First, in analogy to the synthesis described in Intermediate 74, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and tert-butyl-L-phenylalaninate hydrochloride in the presence of O-(7-azabenzotriazol-1-yl)-N,N,N′,N′-tetramethyluronium hexafluorophosphate and subsequent detachment of the Boc protecting group by means of trifluoroacetic acid to obtain the tert-butyl ester (stirring with trifluoroacetic acid in dichloromethane for 40 minutes), the amine compound N-methyl-L-valyl-N-[(3R,4S,5S)-1-{(2S)-2-[(1R,2R)-3-{[(2S)-1-tert-butoxy-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 22 mg (0.02 mmol) of this compound were then used, in analogy to the preparation of Intermediate 61, by reaction with 4-oxobutanoic acid in the presence of sodium cyanoborohydride, to obtain 16 mg (94% of theory) of the title compound.

HPLC (Method 5): Rₜ=2.0 min;

LC-MS (Method 9): Rₜ=5.05 min; MS (ESIpos): m/z=874 (M+H)⁺.

### Intermediate 87

### 4-{[(2S)-1-{[(2S)-1-{[(3R,4S,5S)-1-{(2S)-2-[(1R,2R)-3-{[(2S)-1-tert-Butoxy-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl](methyl)amino}-3-methylbutan-2-yl]amino}-3-methyl-1-oxobutan-2-yl](methyl)amino}butanoic acid

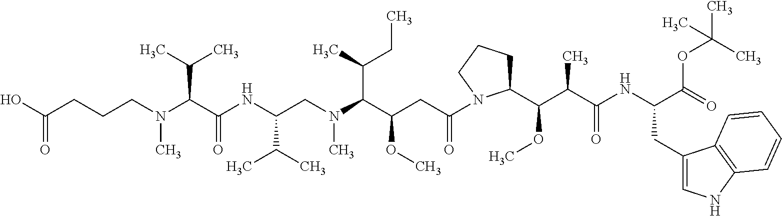 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)OC(C)(C)C)OC)N(C)C[C@@H](NC(=O)[C@H](C(C)C)N(C)CCCC(=O)O)C(C)C]

This compound was prepared in analogy to the synthesis described in Intermediate 86, proceeding from 230 mg (336 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and tert-butyl-L-tryptophanate hydrochloride over 3 stages.

Yield: 95 mg (31% of theory over 3 stages)

HPLC (Method 5): Rₜ=2.0 min;

LC-MS (Method 9): Rₜ=5.05 min; MS (ESIpos): m/z=913 (M+H)⁺.

### Intermediate 88

### N-(6-aminohexyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

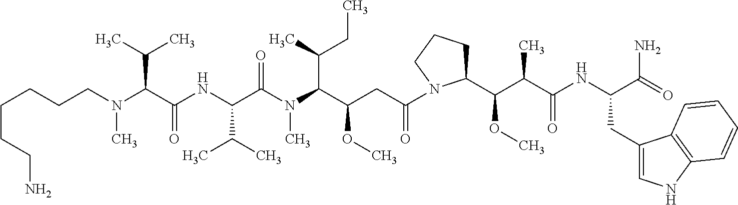 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCCCCN)C(C)C]

First, in analogy to the syntheses described in Intermediate 69, by coupling of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) and N^{α}-{(2R,3R)-3-methoxy-2-methyl-3-[(2S)-pyrrolidin-2-yl]propanoyl}-L-tryptophanamide trifluoroacetate (Intermediate 49) in the presence of O-(7-azabenzotriazol-1-yl)-N,N,N′,N′-tetramethyluronium hexafluorophosphate and subsequent detachment of the Fmoc protecting group by means of piperidine, the amine compound N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 30 mg (0.03 mmol) of this compound were then used, in analogy to the preparation of the compound of Intermediate 61, by reaction with benzyl 6-oxohexyl carbamate, which had been obtained beforehand by oxidation of benzyl 6-hydroxyhexyl carbamate, in the presence of sodium cyanoborohydride, to obtain 17 mg (45% of theory) of the Z-protected compound. Subsequently, hydrogenolysis in methanol over 10% palladium/activated carbon afforded the title compound.

Yield: 14 mg (95% of theory)

HPLC (Method 5): Rₜ=1.5 min;

LC-MS (Method 1): Rₜ=0.73 min; MS (ESIpos): m/z=869 (M+H)⁺.

### Intermediate 89

### N-(6-aminohexyl)-N-methyl-L-valyl-N-[(3R,4S,5S)-1-{(2S)-2-[(1R,2R)-3-{[(2S)-1-tert-butoxy-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

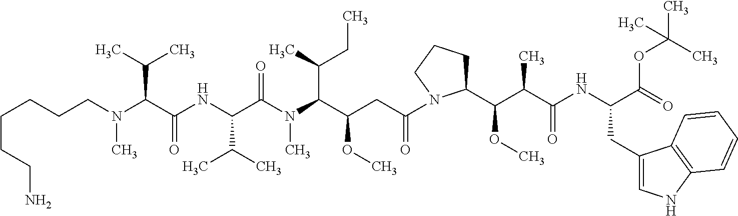 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)OC(C)(C)C)OC)N(C)C(=O)[C@@H](NC(=O)[C@H](C(C)C)N(C)CCCCCCN)C(C)C]

First, in analogy to the synthesis described in Intermediate 86, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and tert-butyl-L-tryptophanate hydrochloride in the presence of O-(7-azabenzotriazol-1-yl)-N,N,N′,N′-tetramethyluronium hexafluorophosphate and subsequent detachment of the Boc protecting group by means of trifluoroacetic acid to obtain the tert-butyl ester (stirring with 1:10 trifluoroacetic acid/dichloromethane for 30 min), the amine compound N-methyl-L-valyl-N-[(3R,4S,5S)-1-{(2S)-2-[(1R,2R)-3-{[(2S)-1-tert-butoxy-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 71 mg (0.075 mmol) of this compound were then used, in analogy to the preparation of the compound of Intermediate 61, by reaction with benzyl 6-oxohexyl carbamate, which had been obtained beforehand by oxidation of benzyl 6-hydroxyhexyl carbamate, in the presence of sodium cyanoborohydride, to obtain 35 mg (44% of theory) of the Z-protected compound. Subsequently, hydrogenolysis in methanol over 10% palladium/activated carbon afforded the title compound.

Yield: 30 mg (98% of theory)

HPLC (Method 5): Rt=1.9 min;

LC-MS (Method 1): Rt=0.77 min; MS (ESIpos): m/z=926 (M+H)+.

### Intermediate 90

### N-(3-carboxypropyl)-N-methyl-L-valyl-N-[(3R,4S,5S)-1-{(2S)-2-[(1R,2R)-3-{[2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

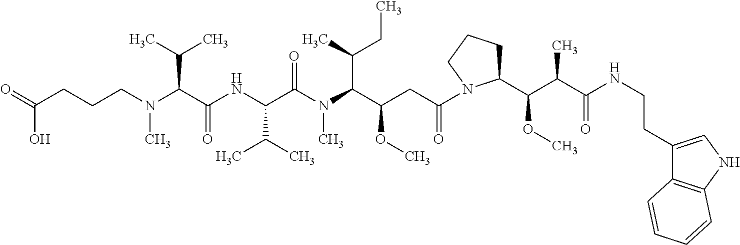 [SMILES: CC[C@H](C)[C@@H]([C@@H](CC(=O)N1CCC[C@H]1[C@H](OC)[C@@H](C)C(=O)NCCc1c[nH]c2ccccc12)OC)N(C)C(=O)[C@@H](NC(=O)[C@H](C(C)C)N(C)CCCC(=O)O)C(C)C]

First, in analogy to the synthesis described in Intermediate 74, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and 2-(1H-indol-3-yl)ethanamine in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-[(3R,4S,5S)-1-{(2S)-2-[(1R,2R)-3-{[2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate. 100 mg (0.119 mmol) of this compound were then used, in analogy to the preparation of Intermediate 61, by reaction with 4-oxobutanoic acid in the presence of sodium cyanoborohydride, to obtain 50 mg (49% of theory) of the title compound. The title compound was purified here by flash chromatography on silica gel with dichloromethane/methanol/17% ammonia as the eluent, in the course of which the mixing ratio was switched from initially 15/2/02 to 15/4/0.5.

HPLC (Method 6): Rₜ=1.8 min;

LC-MS (Method 1): Rₜ=0.87 min; MS (ESIpos): m/z=813 (M+H)⁺.

### Intermediate 91

### N-(3-carboxypropyl)-N-methyl-L-valyl-N-{(3R,4S,5S)-3-methoxy-1-[(2S)-2-{(1R,2R)-1-methoxy-2-methyl-3-oxo-3-[(2-phenylethyl)amino]propyl}pyrrolidin-1-yl]-5-methyl-1-oxoheptan-4-yl}-N-methyl-L-valinamide

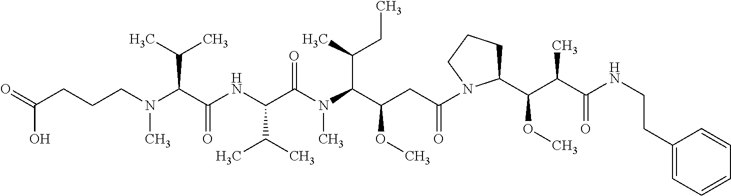 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](C(C)C)N(C)CCCC(=O)O)C(C)C]

First, in analogy to the synthesis described in Intermediate 74, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and phenylethylamine in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-{(3R,4S,5S)-3-methoxy-1-[(2S)-2-{(1R,2R)-1-methoxy-2-methyl-3-oxo-3-[(2-phenylethyl)amino]propyl}pyrrolidin-1-yl]-5-methyl-1-oxoheptan-4-yl}-N-methyl-L-valinamide was prepared as the trifluoroacetate. 57 mg (0.071 mmol) of this compound were then used, in analogy to the preparation of Intermediate 61, by reaction with 4-oxobutanoic acid in the presence of sodium cyanoborohydride, to obtain 44 mg (80% of theory) of the title compound. The title compound can also be purified here by flash chromatography on silica gel with dichloromethane/methanol/17% ammonia as the eluent (15/2/02->15/4/0.5).

HPLC (Method 5): Rₜ=1.7 min;

LC-MS (Method 9): Rₜ=4.64 min; MS (ESIpos): m/z=774 (M+H)⁺.

### Intermediate 92

### N-(3-carboxypropyl)-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

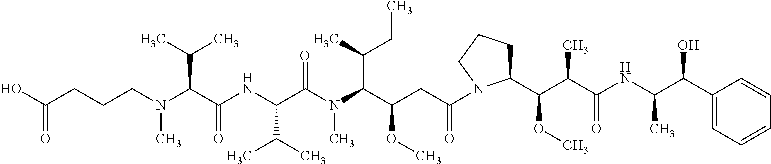 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)CCCC(=O)O)C(C)C]

100 mg (0.139 mmol) of N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 40) were used, in analogy to the preparation of Intermediate 61, by reaction with 4-oxobutanoic acid in the presence of sodium cyanoborohydride, to obtain 94 mg (84% of theory) of the title compound. The title compound was purified by preparative HPLC.

HPLC (Method 5): Rₜ=1.5 min;

LC-MS (Method 9): Rₜ=4.46 min; MS (ESIpos): m/z=804 (M+H)⁺.

### Intermediate 93

### N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

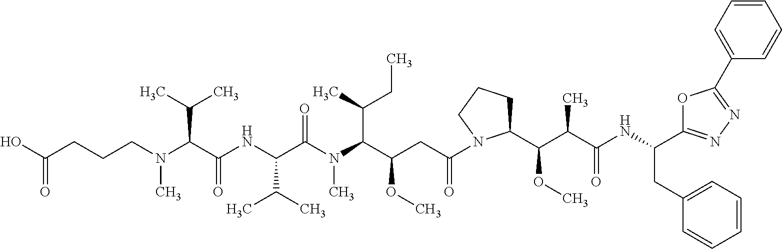 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCC(=O)O)C(C)C]

22.4 mg (24 μmol) of N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide trifluoroacetate were dissolved in 1.4 ml of dioxane/water and, analogously to the preparation of Intermediate 61, reacted with 15% aqueous solution of 4-oxobutanoic acid in the presence of sodium cyanoborohydride. After lyophilization from dioxane, 8.2 mg (38% of theory) of the title compound were obtained in the form of a white solid.

HPLC (Method 10): Rₜ=2.54 min

LC-MS (Method 12): Rₜ=0.94 min; MS (ESIpos): m/z=919 (M+H)⁺

### Intermediate 94

### N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(1R)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

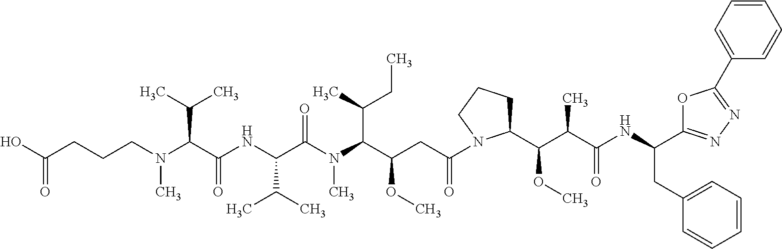 [SMILES: CC[C@H](C)[C@@H]([C@@H](CC(=O)N1CCC[C@H]1[C@H](OC)[C@@H](C)C(=O)N[C@H](Cc1ccccc1)c1nnc(-c2ccccc2)o1)OC)N(C)C(=O)[C@@H](NC(=O)[C@H](C(C)C)N(C)CCCC(=O)O)C(C)C]

17.1 mg (18 μmol) of N-methyl-L-valyl-N-[(3R,4S,5S)-3-methoxy-1-{(2S)-2-[(1R,2R)-1-methoxy-2-methyl-3-oxo-3-{[(1R)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide trifluoroacetate were dissolved in 1.1 ml of dioxane/water and, analogously to the preparation of Intermediate 61, reacted with 15% aqueous solution of 4-oxobutanoic acid in the presence of sodium cyanoborohydride. After lyophilization from dioxane, 14.8 mg (89% of theory) of the title compound were obtained in the form of a white solid.

HPLC (Method 10): Rₜ=2.54 min;

LC-MS (Method 12): Rₜ=0.92 min; MS (ESIpos): m/z=919 (M+H)⁺

### Intermediate 95

### N-(3-carboxypropyl)-N-methyl-L-valyl-N-[(3R,4S,5S)-1-{(2S)-2-[(1R,2R)-3-{[(2S)-1-(benzylsulphonyl)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

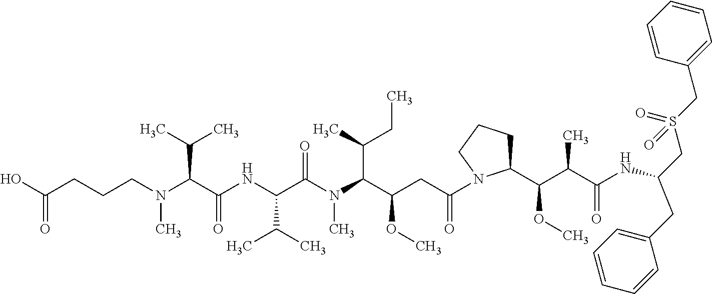 [SMILES: CC[C@H](C)[C@@H]([C@@H](CC(=O)N1CCC[C@H]1[C@H](OC)[C@@H](C)C(=O)N[C@@H](Cc1ccccc1)CS(=O)(=O)Cc1ccccc1)OC)N(C)C(=O)[C@@H](NC(=O)[C@H](C(C)C)N(C)CCCC(=O)O)C(C)C]

19.3 mg (20 μmol) N-methyl-L-valyl-N-[(3R,4S,5S)-1-{(2S)-2-[(1R,2R)-3-{[(2S)-1-(benzylsulphonyl)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate were dissolved in 1.2 ml of dioxane/water and, analogously to the preparation of Intermediate 61, reacted with 15% aqueous solution of 4-oxobutanoic acid in the presence of sodium cyanoborohydride. After lyophilization from dioxane, 8.6 mg (45% of theory) of the title compound were obtained in the form of a solid.

LC-MS (Method 11): Rₜ=0.85 min; MS (ESIpos): m/z=943 (M+H)⁺

### Intermediate 96

### N-(3-carboxypropyl)-N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide

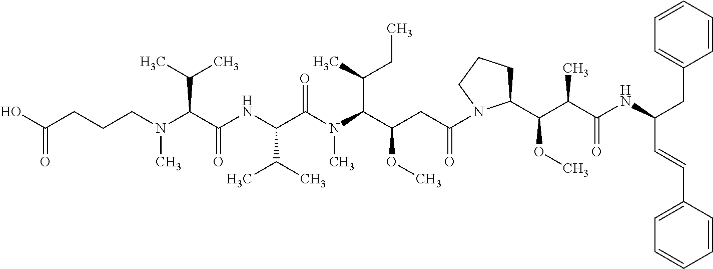 [SMILES: CC[C@H](C)[C@@H]([C@@H](CC(=O)N1CCC[C@H]1[C@H](OC)[C@@H](C)C(=O)N[C@H](/C=C/c1ccccc1)Cc1ccccc1)OC)N(C)C(=O)[C@@H](NC(=O)[C@H](C(C)C)N(C)CCCC(=O)O)C(C)C]

5.5 mg (10 μmol) of N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate were dissolved in 1.0 ml of dioxane/water and, analogously to the preparation of Intermediate 61, reacted with 15% aqueous solution of 4-oxobutanoic acid in the presence of sodium cyanoborohydride. After lyophilization from dioxane, 10.3 mg (68% of theory) of the title compound were obtained in the form of a white solid.

HPLC (Method 10): Rₜ=2.59 min;

LC-MS (Method 11): Rₜ=0.94 min; MS (ESIpos): m/z=877 (M+H)⁺

### Intermediate 97

### N-(6-aminohexyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

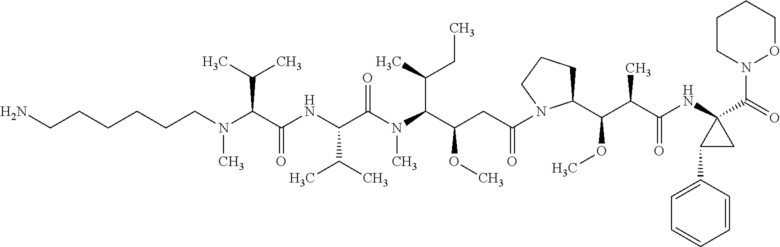 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCCCCN)C(C)C]

The title compound was prepared in analogy to the synthesis of Intermediate 66, by reaction of 200 mg (0.108 mmol) of N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide trifluoroacetate (Intermediate 16) with benzyl 3-oxohexyl carbamate and subsequent hydrogenolytic detachment of the Z protecting group (with 5% palladium on charcoal as a catalyst, in methanol as a solvent).

Yield: 69 mg (65% of theory over two stages)

HPLC (Method 5): Rₜ=1.7 min;

LC-MS (Method 1): Rₜ=0.76 min; MS (ESIpos): m/z=912 (M+H)⁺.

### Intermediate 98

### N-(5-carboxypentyl)-N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

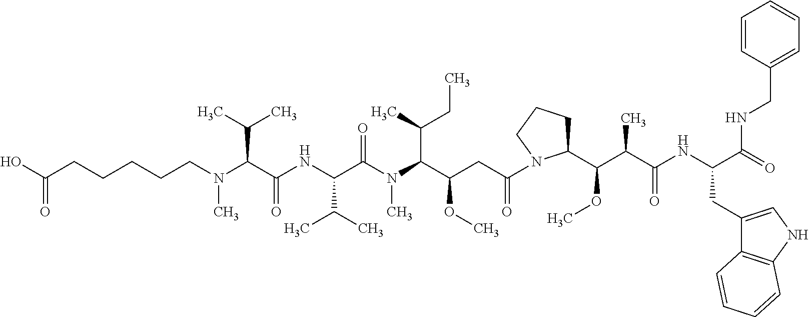 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)NCc1ccccc1)OC)N(C)C(=O)[C@@H](NC(=O)[C@H](C(C)C)N(C)CCCCCC(=O)O)C(C)C]

This compound was prepared in analogy to the synthesis described in Intermediate 80. The purification was effected by preparative HPLC.

Yield: 40 mg (29% of theory over 3 stages)

HPLC (Method 5): Rₜ=1.9 min;

LC-MS (Method 1): Rₜ=0.92 min; MS (ESIpos): m/z=974 (M+H)⁺.

### Intermediate 99

### (2S)-2-amino-3-(1H-indol-3-yl)-1-(1,2-oxazinan-2-yl)propan-1-one trifluoroacetate

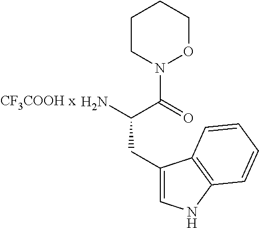 [SMILES: N[C@@H](Cc1c[nH]c2ccccc12)C(=O)N1CCCCO1]

324 mg (0.81 mmol) of 2,5-dioxopyrrolidin-1-yl N-(tert-butoxycarbonyl)-L-tryptophanate were dissolved in 20 ml of DMF, and 200 mg (1.62 mmol) of 1,2-oxazinane hydrochloride (Starting Compound 5) and 850 μl of N,N-diisopropylethylamine were added. The reaction mixture was stirred at 50° C. overnight and then concentrated under reduced pressure. The residue was taken up in dichloromethane and extracted with water. The organic phase was dried over magnesium sulphate and concentrated. The residue was purified by flash chromatography on silica gel with 4:1 dichloromethane/ethyl acetate as the eluent. The product fractions were concentrated and the residue was dried under high vacuum. This gave 147.5 mg (48% of theory) of the Boc-protected intermediate.

HPLC (Method 12): Rₜ=1.9 min;

LC-MS (Method 1): Rₜ=1.03 min; MS (ESIpos): m/z=374 (M+H)⁺.

Using 166 mg (444.5 μmol) of this intermediate, under standard conditions with 3 ml of trifluoroacetic acid in 20 ml of dichloromethane, the Boc protecting group was detached and, after HPLC purification, 155 mg (86% of theory) of the title compound were obtained.

HPLC (Method 12): Rₜ=1.43 min;

LC-MS (Method 11): Rₜ=0.56 min; MS (ESIpos): m/z=274 (M+H)⁺.

### Intermediate 100

### N-(6-{[(benzyloxy)carbonyl]amino}hexyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

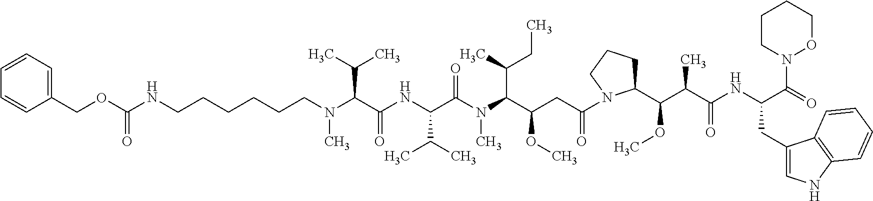 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCCNC(=O)OCc1ccccc1)C(C)C]

77 mg (260 μmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and 100 mg (260 μmol) of (2S)-2-amino-3-(1H-indol-3-yl)-1-(1,2-oxazinan-2-yl)propan-1-one trifluoroacetate (Intermediate 99) were taken up in 15 ml of DMF, and 118 mg (310 μmol) of O-(7-azabenzotriazol-1-yl)-N,N,N′,N′-tetramethyluronium hexafluorophosphate and 140 μl of N,N-diisopropylethylamine were added. The reaction mixture was stirred at RT for 30 min, then concentrated under reduced pressure, and the residue was purified by means of preparative HPLC. The product fractions were combined and concentrated. After lyophilization from dioxane, 170 mg (68% of theory) of the Boc-protected intermediate were obtained.

LC-MS (Method 1): Rₜ=1.36 min; m/z=940 (M+H)⁺.

170 mg of this intermediate were treated with 3 ml of trifluoroacetic acid in 30 ml of dichloromethane for 30 min to detach the Boc protecting group. Then the reaction mixture was concentrated under reduced pressure and the residue was purified by means of preparative HPLC to obtain 155 mg (86% of theory) of the deprotected N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide intermediate.

HPLC (Method 12): Rₜ=1.85 min;

LC-MS (Method 1): Rₜ=0.86 min; MS (ESIpos): m/z=840 (M+H)⁺.

50 mg (0.052 mmol) of this intermediate were then used, in analogy to the preparation of Intermediate 97, with benzyl 3-oxohexyl carbamate in the presence of sodium cyanoborohydride and subsequent hydrogenolytic detachment of the Z protecting group (with 5% palladium on charcoal as a catalyst, in methanol as a solvent), prepared to prepare the title compound.

Yield: 21 mg (37% of theory)

HPLC (Method 12): Rₜ=2.1 min;

LC-MS (Method 1): Rₜ=1.02 min; MS (ESIpos): m/z=1073 (M+H)⁺.

### Intermediate 101

### N-(6-aminohexyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

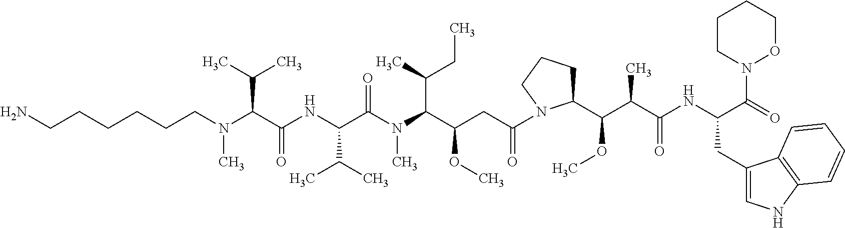 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCCN)C(C)C]

26.7 mg (24.87 μmol) of Intermediate 100 were dissolved in 10 ml of methanol and hydrogenated over palladium/activated carbon (5%) under standard hydrogen pressure for 30 min. The catalyst was filtered off and the solvent was evaporated off under reduced pressure. After the residue had been dried under high vacuum, 22.5 mg (96% of theory) of the title compound were obtained.

HPLC (Method 5): Rₜ=1.7 min;

LC-MS (Method 1): Rₜ=0.76 min; MS (ESIpos): m/z=939 (M+H)⁺.

### Intermediate 102

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-{[(2S)-1-(morpholin-4-yl)-1-oxo-3-phenylpropan-2-yl]amino}-3-oxopropyl]pyrrolidin-1-yl}-5-methyl-1-oxoheptan-4-yl]-N-methyl-L-valinamide

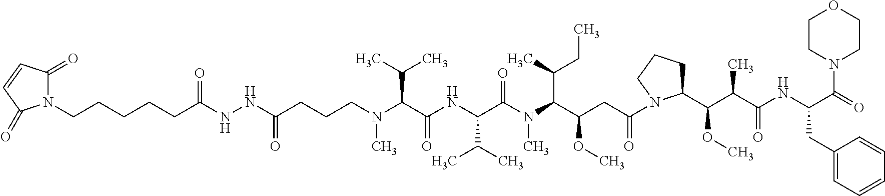 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCOCC1)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 157 from N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(morpholin-4-yl)-1-oxo-3-phenylpropan-2-yl]amino}-3-oxopropyl]pyrrolidin-1-yl}-5-methyl-1-oxoheptan-4-yl]-N-methyl-L-valinamide and commercially available 6-(2,5-dioxo-2,5-dihydro-1H-pyrrol-1-yl)hexanehydrazide.

Yield: 8 mg (71% of theory)

HPLC (Method 12): Rₜ=1.9 min;

LC-MS (Method 1): Rₜ=0.87 min; MS (ESIpos): m/z=1094 (M+H)⁺.

### Intermediate 103

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S,3R)-1-(benzylamino)-3-hydroxy-1-oxobutan-2-yl]amino}-1-methoxy-2-methyl-3-oxopropyl]pyrrolidin-1-yl}-3-methoxy-5-methyl-1-oxoheptan-4-yl]-N-methyl-L-valinamide

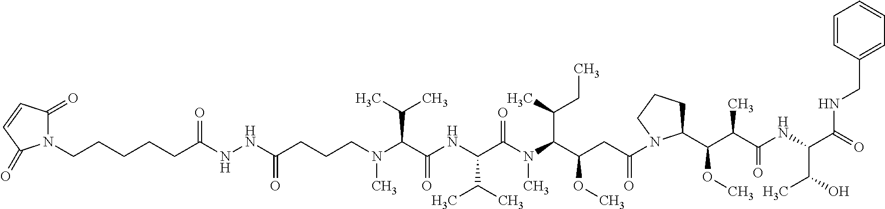 [SMILES: CC[C@H](C)[C@@H]([C@@H](CC(=O)N1CCC[C@H]1[C@H](OC)[C@@H](C)C(=O)N[C@H](C(=O)NCc1ccccc1)[C@@H](C)O)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 157 from N-(3-carboxypropyl)-N-methyl-L-valyl-N-[(3R,4S,5S)-1-{(2S)-2-[(1R,2R)-3-{[(2S,3R)-1-(benzylamino)-3-hydroxy-1-oxobutan-2-yl]amino}-1-methoxy-2-methyl-3-oxopropyl]pyrrolidin-1-yl}-3-methoxy-5-methyl-1-oxoheptan-4-yl]-N-methyl-L-valinamide and commercially available 6-(2,5-dioxo-2,5-dihydro-1H-pyrrol-1-yl)hexanehydrazide.

Yield: 3 mg (22% of theory)

HPLC (Method 5): Rₜ=1.6 min;

LC-MS (Method 1): Rₜ=0.78 min; MS (ESIpos): m/z=1069 (M+H)⁺.

### Intermediate 104

### N-{4-[(trans-4-{[(2,5-dioxopyrrolidin-1-yl)oxy]carbonyl}cyclohexyl)amino]-4-oxobutyl}-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

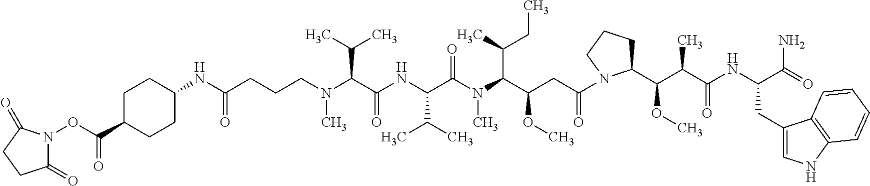 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)N[C@H]1CC[C@H](C(=O)ON2C(=O)CCC2=O)CC1)C(C)C]

First, benzyl trans-4-aminocyclohexanecarboxylate trifluoroacetate was prepared from trans-4-aminocyclohexanecarboxylic acid by introducing the Boc protecting group, then introducing the benzyl ester protecting group and subsequently detaching the Boc protecting group by conventional peptide chemistry methods.

15 mg (18 μmol) of N-(3-carboxypropyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide were then dissolved in 5 ml of dimethylformamide and subsequently admixed with 13 mg (35 μmol) of O-(7-azabenzotriazol-1-yl)-N,N,N′,N′-tetramethyluronium hexafluorophosphate, 9 μl of N,N-diisopropylethylamine and with 15 mg (44 μmol) of benzyl trans-4-aminocyclohexanecarboxylate trifluoroacetate. The mixture was stirred at RT for 1 h and then concentrated under reduced pressure. The remaining residue was purified by means of preparative HPLC. The corresponding fractions were combined and the solvent was evaporated off under reduced pressure. After the residue had been dried under high vacuum, 14.7 mg (78% of theory) of the protected intermediate were obtained as a colourless foam.

HPLC (Method 6): Rₜ=2.0 min;

LC-MS (Method 1): Rₜ=0.95 min; MS (ESIpos): m/z=1072 (M+H)⁺.

From this protected intermediate, the benzyl ester was first removed by hydrogenolytic means and the free carboxyl component was obtained in quantitative yield. 14 mg (14 μmol; 1 equiv.) of the deprotected compound were taken up in 5 ml of DMF and admixed with 3.3 mg (29 μmol; 2.1 equiv.) of N-hydroxysuccinimide in the presence of 4.1 mg (21 μmol; 1.5 equiv.) of 1-(3-dimethylaminopropyl)-3-ethylcarbodiimide hydrochloride, 7.5 μl (44 μmol; 3.1 equiv.) of N,N-diisopropylethylamine and 0.9 mg (7 μmol; 0.5 equiv.) of 4-dimethylaminopyridine, and the mixture was stirred at RT overnight. Then another 10 equiv. of N-hydroxysuccinimide, 5 equiv. of 1-(3-dimethylaminopropyl)-3-ethylcarbodiimide hydrochloride, 5 equiv. of N,N-diisopropylethylamine and 0.5 equiv. of 4-dimethylaminopyridine were added and the reaction mixture was treated in an ultrasound bath for 5 h. Subsequently, the solvent was evaporated off, the residue was purified by means of preparative HPLC and the corresponding fractions were combined and concentrated. After lyophilization of the residue from dioxane, 9.7 mg (62% of theory) of the title compound were obtained as a colourless foam.

HPLC (Method 6): Rₜ=1.8 min;

LC-MS (Method 11): Rₜ=0.77 min; MS (ESIpos): m/z=1078 (M+H)⁺.

### Intermediate 105

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide

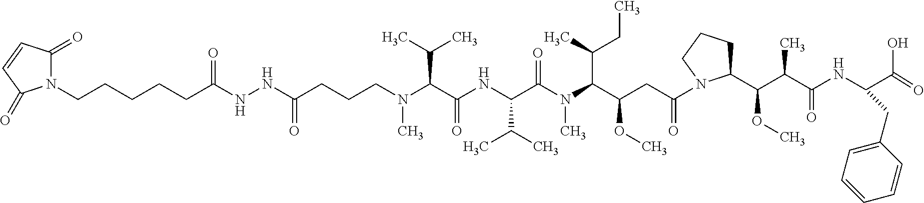

This compound was prepared in analogy to the synthesis described in Intermediate 157, proceeding from 4-{[(2S)-1-{[(2S)-1-{[(3R,4S,5S)-1-{(2S)-2-[(1R,2R)-3-{[(2S)-1-tert-butoxy-1-oxo-3-phenylpropan-2-yl]amino}-1-methoxy-2-methyl-3-oxopropyl]pyrrolidin-1-yl}-3-methoxy-5-methyl-1-oxoheptan-4-yl](methyl)amino}-3-methylbutan-2-yl]amino}-3-methyl-1-oxobutan-2-yl](methyl)amino}butanoic acid and commercially available 6-(2,5-dioxo-2,5-dihydro-1H-pyrrol-1-yl)hexanehydrazide. The ester intermediate was obtained in 42% yield. In a second step, 6 mg (6 μmol) of this intermediate were cleaved with trifluoroacetic acid the tert-butyl ester. After HPLC purification, 3.4 mg (48% of theory) of the title compound were obtained.

HPLC (Method 5): Rₜ=1.66 min;

LC-MS (Method 2): Rₜ=1.04 min; MS (ESIpos): m/z=1025 (M+H)⁺.

### Intermediate 106

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

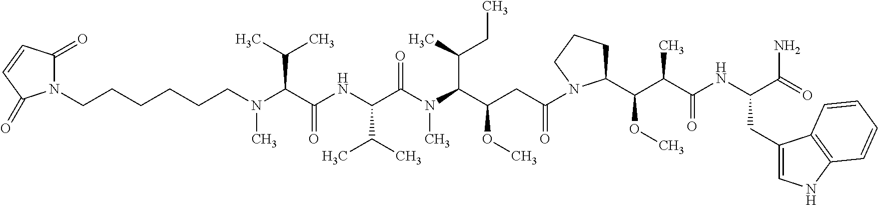 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCCCCN1C(=O)C=CC1=O)C(C)C]

14 mg (16 μmol) of N-(6-aminohexyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 87) were taken up in 750 μl of dioxane and admixed with 1.5 ml of saturated sodium hydrogencarbonate solution and then with 3.2 mg (21 μmol) of methyl 2,5-dioxo-2,5-dihydro-1H-pyrrole-1-carboxylate. The reaction mixture was stirred at RT for 1 h and then concentrated under reduced pressure. The remaining residue was purified by means of preparative HPLC. After lyophilization, 5.5 mg (36% of theory) of the title compound were obtained.

HPLC (Method 5): Rₜ=1.7 min;

LC-MS (Method 1): Rₜ=0.84 min; MS (ESIpos): m/z=949 (M+H)⁺.

### Intermediate 107

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

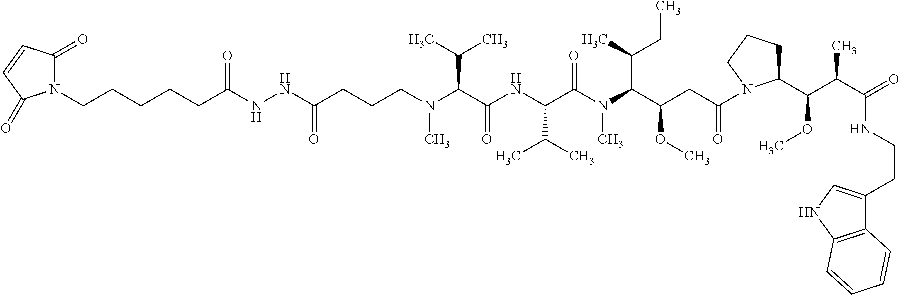 [SMILES: CC[C@H](C)[C@@H]([C@@H](CC(=O)N1CCC[C@H]1[C@H](OC)[C@@H](C)C(=O)NCCc1c[nH]c2ccccc12)OC)N(C)C(=O)[C@@H](NC(=O)[C@H](C(C)C)N(C)CCCC(=O)NNC(=O)CCCCCN1C(=O)C=CC1=O)C(C)C]

38 mg (47 μmol) of N-(3-carboxypropyl)-N-methyl-L-valyl-N-[(3R,4S,5S)-1-{(2S)-2-[(1R,2R)-3-{[2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide were dissolved in 37 ml of DMF and then admixed with 71 mg (187 μmol) of O-(7-azabenzotriazol-1-yl)-N,N,N′,N′-tetramethyluronium hexafluorophosphate, 33 μl of N,N-diisopropylethylamine and with 37 mg (140 μmol) of commercially available 6-(2,5-dioxo-2,5-dihydro-1H-pyrrol-1-yl)hexanehydrazide. The mixture was stirred at RT for 1 h. This was followed by concentration under high vacuum and purification of the remaining residue by means of preparative HPLC. Thus, 12.2 mg (26% of theory) of the title compound were obtained as a colourless foam.

HPLC (Method 5): Rₜ=1.6 min;

LC-MS (Method 1): Rₜ=0.85 min; MS (ESIpos): m/z=1020 (M+H)⁺.

### Intermediate 108

### N-(4-{2-[6-(2,5-dioxo-2,5-dihydro-1H-pyrrol-1-yl)hexanoyl]hydrazino}-4-oxobutyl)-N-methyl-L-valyl-N-{(3R,4S,5S)-3-methoxy-1-[(2S)-2-{(1R,2R)-1-methoxy-2-methyl-3-oxo-3-[(2-phenylethyl)amino]propyl}pyrrolidin-1-yl]-5-methyl-1-oxoheptan-4-yl}-N-methyl-L-valinamide

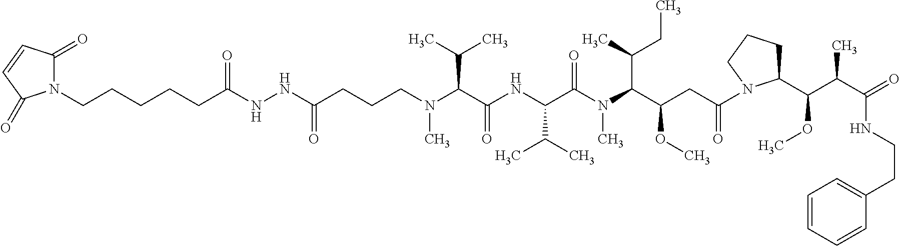 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](C(C)C)N(C)CCCC(=O)NNC(=O)CCCCCN1C(=O)C=CC1=O)C(C)C]

The compound was prepared in analogy to Intermediate 107.

Yield: 2.5 mg (30% of theory)

HPLC (Method 12): Rₜ=1.9 min;

LC-MS (Method 1): Rₜ=0.9 min; MS (ESIpos): m/z=981 (M+H)⁺.

### Intermediate 109

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

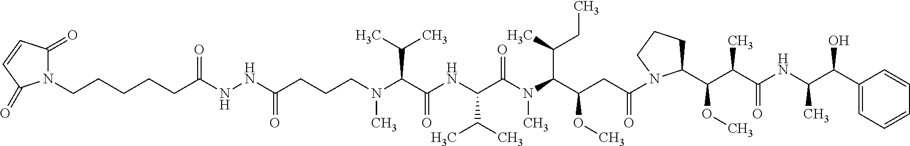 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)CCCC(=O)NNC(=O)CCCCCN1C(=O)C=CC1=O)C(C)C]

The compound was prepared in analogy to Intermediate 107 from the compound in Intermediate 92.

Yield: 35 mg (65% of theory)

HPLC (Method 5): Rₜ=1.9 min;

LC-MS (Method 11): Rₜ=0.76 min; MS (ESIpos): m/z=1011 (M+H)⁺.

### Intermediate 110

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

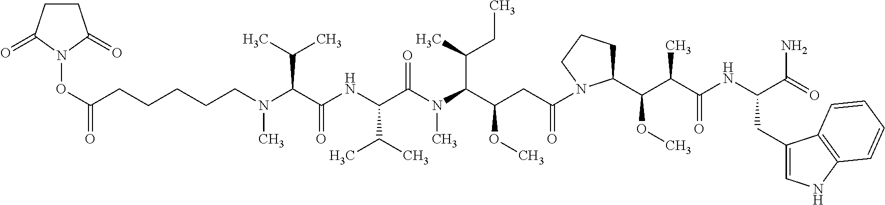 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCCCC(=O)ON1C(=O)CCC1=O)C(C)C]

This compound was prepared in analogy to Intermediate 147 from the compound in Intermediate 83.

Yield: 2.4 mg (24% of theory)

HPLC (Method 6): Rₜ=1.8 min;

LC-MS (Method 1): Rₜ=0.87 min; MS (ESIpos): m/z=981 (M+H)⁺.

### Intermediate 111

### N-(4-{2-[6-(2,5-dioxo-2,5-dihydro-1H-pyrrol-1-yl)hexanoyl]-1-methylhydrazino}-4-oxobutyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

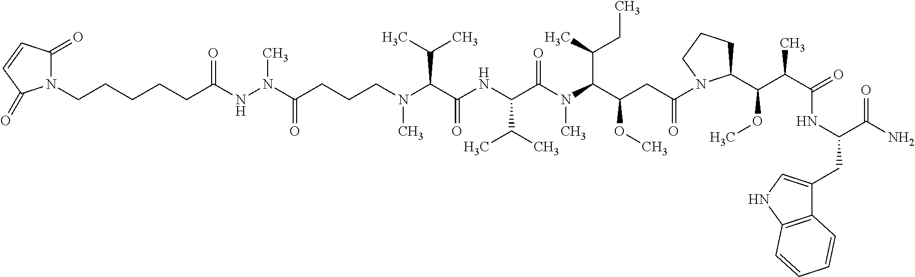 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)N(C)NC(=O)CCCCCN1C(=O)C=CC1=O)C(C)C]

This compound was prepared in analogy to Intermediate 140 from Intermediate 82 and Intermediate 22.

Yield: 6.5 mg (51% of theory)

HPLC (Method 6): Rₜ=1.8 min;

LC-MS (Method 1): Rₜ=4.71 min; MS (ESIpos): m/z=1077 (M+H)⁺.

### Intermediate 112

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(1S,2R)-1-carbamoyl-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

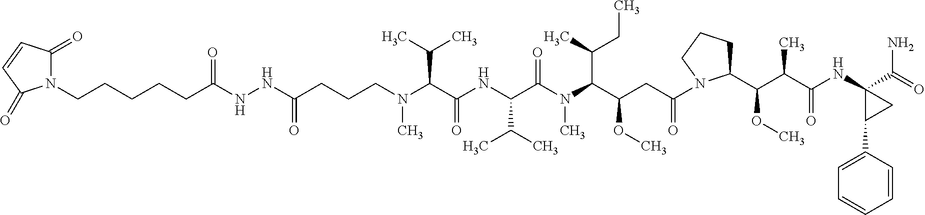 [SMILES: CC[C@H](C)[C@@H]([C@@H](CC(=O)N1CCC[C@H]1[C@H](OC)[C@@H](C)C(=O)N[C@@]1(C(N)=O)C[C@@H]1c1ccccc1)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to Intermediate 157 from the compound in Intermediate 81.

Yield: 5.7 mg (57% of theory)

HPLC (Method 5): Rₜ=1.6 min;

LC-MS (Method 1): Rₜ=0.87 min; MS (ESIpos): m/z=1036 (M+H)⁺.

### Intermediate 113

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(1S)-1-carboxy-2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

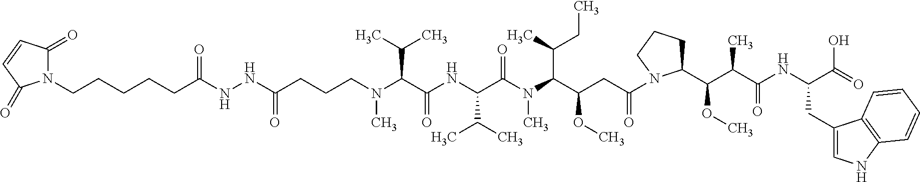 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)O)OC)N(C)C(=O)[C@@H](NC(=O)[C@H](C(C)C)N(C)CCCC(=O)NNC(=O)CCCCCN1C(=O)C=CC1=O)C(C)C]

95 mg (104 μmol) of 4-{[(2S)-1-{[(2S)-1-{[(3R,4S,5S)-1-{(2S)-2-[(1R,2R)-3-{[(2S)-1-tert-butoxy-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl](methyl)amino}-3-methylbutan-2-yl]amino}-3-methyl-1-oxobutan-2-yl](methyl)amino}butanoic acid were dissolved in DMF and then admixed with 79.5 mg (209 μmol) of O-(7-azabenzotriazol-1-yl)-N,N,N′,N′-tetramethyluronium hexafluorophosphate, 73 μl of N,N-diisopropylethylamine and with 68 mg (261 μmol) of commercially available 6-(2,5-dioxo-2,5-dihydro-1H-pyrrol-1-yl)hexanehydrazide. The mixture was stirred at RT for 2 h. This was followed by concentration under high vacuum and purification of the remaining residue by means of preparative HPLC. Thus, 104 mg (89% of theory) of the tert-butyl ester of the title compound were obtained as a colourless foam.

HPLC (Method 5): Rₜ=2.0 min;

LC-MS (Method 1): Rₜ=0.93 min; MS (ESIpos): m/z=1121 (M+H)⁺.

The intermediate was taken up in 33.4 ml of dichloromethane, 17 ml of trifluoroacetic acid were added, and the mixture was stirred at RT for 1 h. Subsequently, the reaction mixture was concentrated under reduced pressure and the residue was purified by preparative HPLC.

Thus, 61 mg (62% of theory) of the title compound were obtained as a colourless foam.

HPLC (Method 5): Rₜ=1.7 min;

LC-MS (Method 1): Rₜ=0.86 min; MS (ESIpos): m/z=1064 (M+H)⁺.

### Intermediate 114

### N-[6-({[2-(2,5-dioxo-2,5-dihydro-1H-pyrrol-1-yl)ethyl]carbamoyl}amino)hexyl]-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

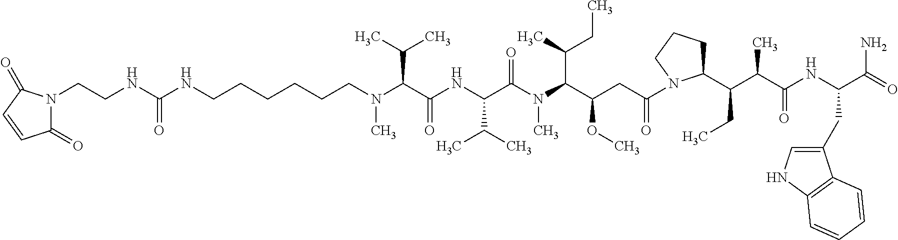 [SMILES: CC[C@H]([C@@H](C)C(=O)N[C@@H](Cc1c[nH]c2ccccc12)C(N)=O)[C@@H]1CCCN1C(=O)C[C@@H](OC)[C@H]([C@@H](C)CC)N(C)C(=O)[C@@H](NC(=O)[C@H](C(C)C)N(C)CCCCCCNC(=O)NCCN1C(=O)C=CC1=O)C(C)C]

5 mg (5 μmol) of N-(6-aminohexyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide were taken up in 885 μl of DMF and admixed with 5.3 mg (8 μmol) of 4-nitrophenyl 2-(2,5-dioxo-2,5-dihydro-1H-pyrrol-1-yl)ethyl carbamate and 2.8 μl of N,N-diisopropylethylamine. The reaction mixture was stirred at RT for 2 h and then concentrated to dryness. The residue was purified by means of preparative HPLC.

Yield: 0.58 mg (11% of theory) of a colourless foam

HPLC (Method 5): Rₜ=1.6 min;

LC-MS (Method 1): Rₜ=0.83 min; MS (ESIpos): m/z=1035 (M+H)⁺.

### Intermediate 115

### N-{4-[(2,5-dioxopyrrolidin-1-yl)oxy]-4-oxobutyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

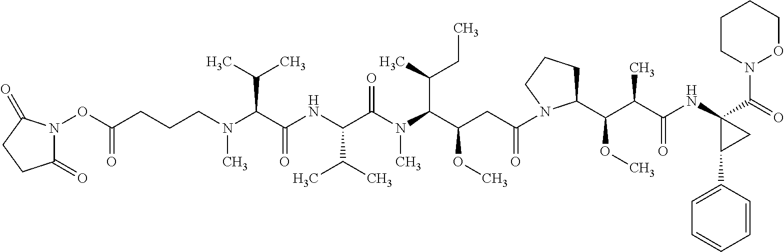 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)ON1C(=O)CCC1=O)C(C)C]

This compound was prepared in analogy to the compound in Intermediate 147, proceeding from 8 mg (9 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide After concentration, the activated ester was purified by means of preparative HPLC and, after removal of the solvent under reduced pressure, reacted immediately with the antibody.

Yield: 3 mg (27% of theory) (hydrolysis-sensitive)

HPLC (Method 5): Rₜ=1.7 min;

LC-MS (Method 1): Rₜ=0.87 min; MS (ESIpos): m/z=996 (M+H)⁺.

### Intermediate 116

### N-{4-[(2,5-dioxopyrrolidin-1-yl)oxy]-4-oxobutyl}-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

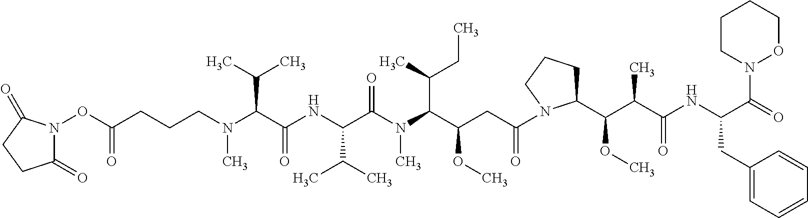 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCC(=O)ON1C(=O)CCC1=O)C(C)C]

This compound was prepared in analogy to the compound in Intermediate 147, proceeding from 5 mg (6 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide After concentration, the activated ester was purified by means of preparative HPLC and, after removal of the solvent under reduced pressure, reacted immediately with the antibody.

Yield: 3.2 mg (43% of theory) (hydrolysis-sensitive)

HPLC (Method 5): Rₜ=1.7 min;

LC-MS (Method 1): Rₜ=0.92 min; MS (ESIpos): m/z=984 (M+H)⁺.

### Intermediate 117

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-tert-butoxy-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

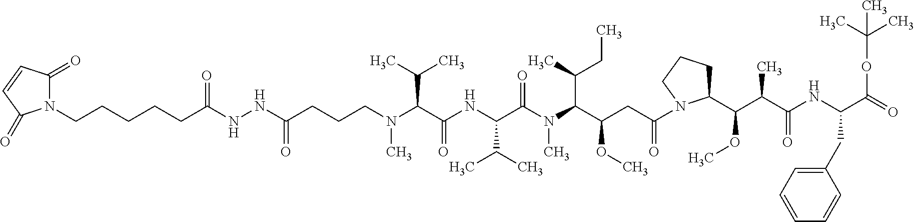 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC(C)(C)C)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to Intermediate 157 from the compound in Intermediate 86.

Yield: 7 mg (42% of theory)

HPLC (Method 5): Rₜ=1.6 min;

LC-MS (Method 1): Rₜ=0.94 min; MS (ESIpos): m/z=1081 (M+H)⁺.

### Intermediate 118

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2R)-1-(benzyloxy)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

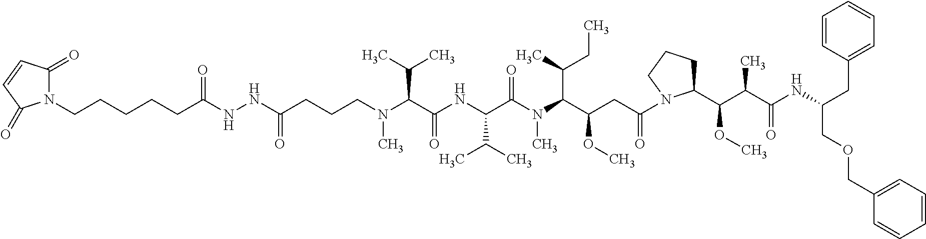 [SMILES: CC[C@H](C)[C@@H]([C@@H](CC(=O)N1CCC[C@H]1[C@H](OC)[C@@H](C)C(=O)N[C@@H](COCc1ccccc1)Cc1ccccc1)OC)N(C)C(=O)[C@@H](NC(=O)[C@H](C(C)C)N(C)CCCC(=O)NNC(=O)CCCCCN1C(=O)C=CC1=O)C(C)C]

The target compound was prepared analogously to Intermediate 157 from 7 mg (7.8 μmol) of the compound in Intermediate 68. Yield: 6.3 mg (53% of theory)

LC-MS (Method 1): Rₜ=1.00 min; MS (ESIpos): m/z=1102 (M+H)⁺.

### Intermediate 119

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

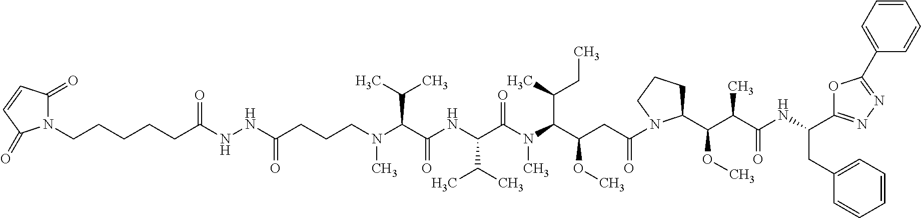 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCC(=O)NNC(=O)CCCCCN1C(=O)C=CC1=O)C(C)C]

7.4 mg (8.1 mmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide and 6.3 mg (24.2 mmol) of 6-(2,5-dioxo-2,5-dihydro-1H-pyrrol-1-yl)hexanehydrazide hydrochloride were coupled and worked up in analogy to Intermediate 157. 1.6 mg (13% of theory) of the title compound were obtained as a solid.

LC-MS (Method 11): Rₜ=0.89 min; MS (ESIpos): m/z=1126 (M+H)⁺

### Intermediate 120

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-oxo-3-{[(1R)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

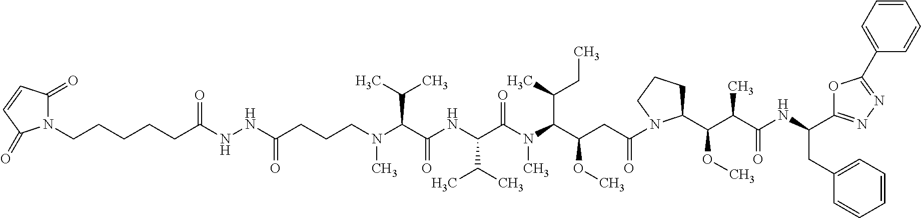 [SMILES: CC[C@H](C)[C@@H]([C@@H](CC(=O)N1CCC[C@H]1[C@H](OC)[C@@H](C)C(=O)N[C@H](Cc1ccccc1)c1nnc(-c2ccccc2)o1)OC)N(C)C(=O)[C@@H](NC(=O)[C@H](C(C)C)N(C)CCCC(=O)NNC(=O)CCCCCN1C(=O)C=CC1=O)C(C)C]

12.8 mg (13.9 mmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(1R)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide and 10.9 mg (41.8 mmol) of 6-(2,5-dioxo-2,5-dihydro-1H-pyrrol-1-yl)hexanehydrazide hydrochloride were coupled and worked up in analogy to Intermediate 157. 10.8 mg (59% of theory) of the title compound were obtained as a solid.

LC-MS (Method 11): Rₜ=0.90 min; MS (ESIpos): m/z=1126 (M+H)⁺

### Intermediate 121

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-(benzylsulphonyl)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

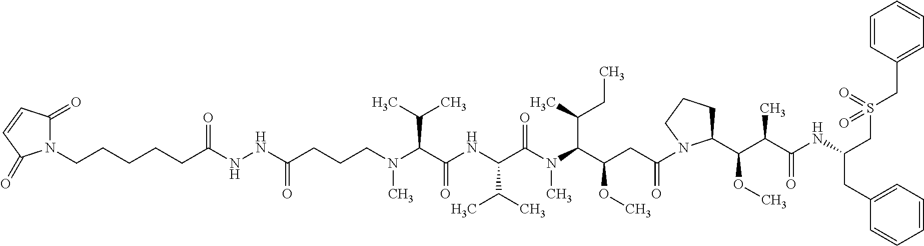 [SMILES: CC[C@H](C)[C@@H]([C@@H](CC(=O)N1CCC[C@H]1[C@H](OC)[C@@H](C)C(=O)N[C@@H](Cc1ccccc1)CS(=O)(=O)Cc1ccccc1)OC)N(C)C(=O)[C@@H](NC(=O)[C@H](C(C)C)N(C)CCCC(=O)NNC(=O)CCCCCN1C(=O)C=CC1=O)C(C)C]

7.4 mg (7.9 mmol) of N-(3-carboxypropyl)-N-methyl-L-valyl-N-[(3R,4S,5S)-1-{(2S)-2-[(1R,2R)-3-{[(2S)-1-(benzylsulphonyl)-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide and 6.2 mg (23.5 mmol) of 6-(2,5-dioxo-2,5-dihydro-1H-pyrrol-1-yl)hexanehydrazide hydrochloride were coupled and worked up in analogy to Intermediate 157. 6.9 mg (74% of theory) of the title compound were obtained as a solid.

LC-MS (Method 11): Rₜ=0.87 min; MS (ESIpos): m/z=1150 (M+H)⁺

### Intermediate 122

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide

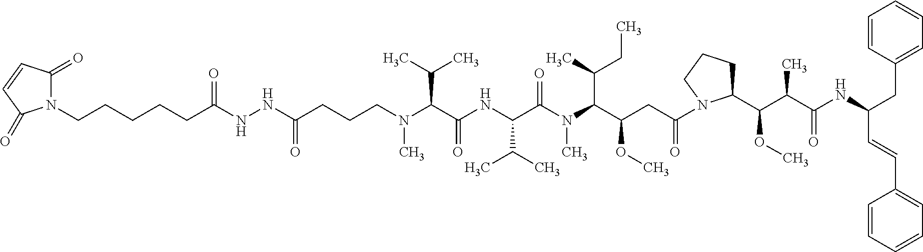 [SMILES: CC[C@H](C)[C@@H]([C@@H](CC(=O)N1CCC[C@H]1[C@H](OC)[C@@H](C)C(=O)N[C@H](/C=C/c1ccccc1)Cc1ccccc1)OC)N(C)C(=O)[C@@H](NC(=O)[C@H](C(C)C)N(C)CCCC(=O)NNC(=O)CCCCCN1C(=O)C=CC1=O)C(C)C]

8 mg (9.1 mmol) of N-(3-carboxypropyl)-N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide and 7.2 mg (27.4 mmol) of 6-(2,5-dioxo-2,5-dihydro-1H-pyrrol-1-yl)hexanehydrazide hydrochloride were coupled and worked up in analogy to Intermediate 157. 8.2 mg (82% of theory) of the title compound were obtained as a white solid.

LC-MS (Method 11): Rₜ=0.95 min; MS (ESIpos): m/z=1083 (M+H)⁺

### Intermediate 123

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(2S)-1-tert-butoxy-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

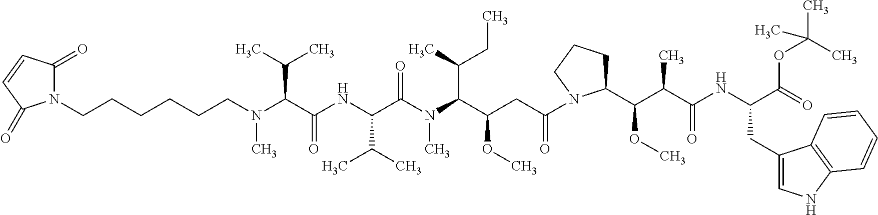 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)OC(C)(C)C)OC)N(C)C(=O)[C@@H](NC(=O)[C@H](C(C)C)N(C)CCCCCCN1C(=O)C=CC1=O)C(C)C]

30 mg (30 μmol) of Intermediate 89 were taken up in 2 ml of 1,4-dioxane and admixed with 4 ml of saturated sodium hydrogencarbonate solution and then with 7.5 mg (50 μmol) of methyl 2,5-dioxo-2,5-dihydro-1H-pyrrole-1-carboxylate. The reaction mixture was stirred at RT for 1 h and then concentrated under reduced pressure. The remaining residue was purified by means of preparative HPLC. After lyophilization, 24 mg (74% of theory) of the title compound were obtained.

HPLC (Method 5): Rₜ=2.2 min;

LC-MS (Method 1): Rₜ=1.01 min; MS (ESIpos): m/z=1006 (M+H)⁺.

### Intermediate 124

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(1S)-1-carboxy-2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

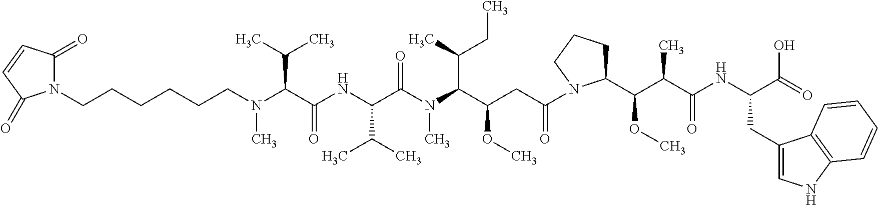 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)O)OC)N(C)C(=O)[C@@H](NC(=O)[C@H](C(C)C)N(C)CCCCCCN1C(=O)C=CC1=O)C(C)C]

22 mg (20 μmol) of Intermediate 123 were reacted with 4 ml of trifluoroacetic acid in 8 ml of dichloromethane at RT for 1 h. Thereafter, the reaction mixture was concentrated under reduced pressure. The remaining residue was purified by means of preparative HPLC. After lyophilization, 11 mg (54% of theory) of the title compound were obtained.

HPLC (Method 5): Rₜ=1.8 min;

LC-MS (Method 11): Rₜ=0.85 min; MS (ESIpos): m/z=950 (M+H)⁺.

### Intermediate 125

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

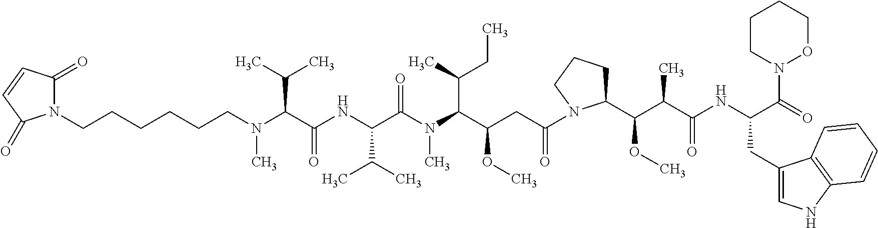 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCCN1C(=O)C=CC1=O)C(C)C]

22.5 mg (20 μmol) of Intermediate 101 were taken up in 2 ml of 1:1 dioxane/water and then admixed with 5.6 mg (40 μmol) of methyl 2,5-dioxo-2,5-dihydro-1H-pyrrole-1-carboxylate and with 0.25 ml of saturated sodium hydrogencarbonate solution. The reaction mixture was stirred at RT for 30 min. Then another 0.25 ml of the saturated sodium hydrogencarbonate solution was added and the reaction mixture was stirred at RT for a further 15 min and then concentrated under reduced pressure. The remaining residue was purified by means of preparative HPLC. After lyophilization, 12.8 mg (50% of theory) of the title compound were obtained as a colourless foam.

HPLC (Method 5): Rₜ=1.9 min;

LC-MS (Method 1): Rₜ=0.95 min; MS (ESIpos): m/z=1019 (M+H)⁺.

### Intermediate 126

### N-[6-(2,5-dioxo-2,5-dihydro-1H-pyrrol-1-yl)hexyl]-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

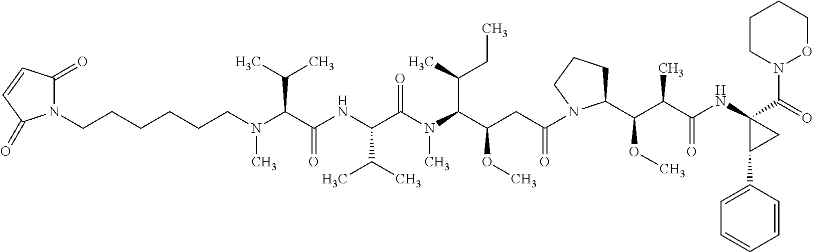 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCCCCN1C(=O)C=CC1=O)C(C)C]

64 mg (70 μmol) of N-(6-aminohexyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide (Intermediate 97) were taken up in 3 ml of 1:1 dioxane/water, then adjusted to pH 9 with 4 ml of saturated sodium hydrogencarbonate solution and subsequently admixed with 16.3 mg (110 μmol) of methyl 2,5-dioxo-2,5-dihydro-1H-pyrrole-1-carboxylate. The reaction mixture was stirred at RT for 1 h and then concentrated under reduced pressure. Then another 8 mg (55 μmol) of methyl 2,5-dioxo-2,5-dihydro-1H-pyrrole-1-carboxylate were added, and the reaction mixture was adjusted again to pH 9 and stirred at RT for a further hour. This was followed by concentration and purification of the remaining residue by means of preparative HPLC. At first, 31 mg of an as yet uncyclized intermediate were obtained. 27 mg of this intermediate were taken up again in 2 ml of 1:1 dioxane/water and then admixed with 250 μl of saturated sodium hydrogencarbonate solution. After stirring at RT for 2 hours, the reaction mixture was concentrated and the residue was purified by means of preparative HPLC. After lyophilization, 20 mg (29% of theory) of the title compound were obtained.

HPLC (Method 5): Rₜ=1.96 min;

LC-MS (Method 1): Rₜ=0.97 min; MS (ESIpos): m/z=992 (M+H)⁺.

### Intermediate 127

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

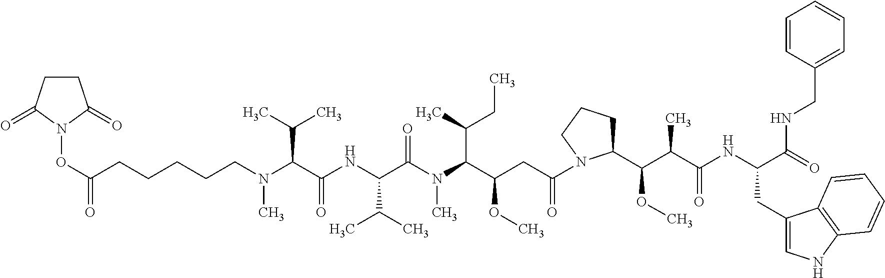 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)NCc1ccccc1)OC)N(C)C(=O)[C@@H](NC(=O)[C@H](C(C)C)N(C)CCCCCC(=O)ON1C(=O)CCC1=O)C(C)C]

17 mg (18 μmol) of N-(5-carboxypentyl)-N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 98) were dissolved in 2.8 ml of dichloromethane and admixed with 20 mg (174 mmol) of 1-hydroxypyrrolidine-2,5-dione and then with 10 mg (52 μmol) of 1-(3-dimethylaminopropyl)-3-ethylcarbodiimide hydrochloride and 0.21 mg (0.17 μmol) of DMAP. After stirring at RT for 4 h, the reaction mixture was concentrated under reduced pressure. The remaining residue was purified by means of preparative HPLC. After lyophilization, 8.2 mg (43% of theory) of the title compound were obtained.

HPLC (Method 5): Rₜ=2.0 min;

LC-MS (Method 1): Rₜ=0.98 min; MS (ESIpos): m/z=1071 (M+H)⁺.

### Intermediate 128

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

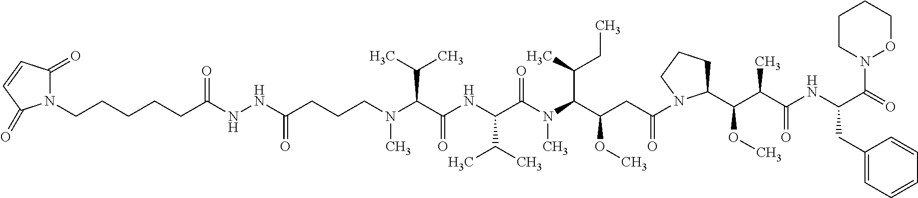 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCC(=O)NNC(=O)CCCCCN1C(=O)C=CC1=O)C(C)C]

5 mg (5.6 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide were dissolved in 845 μl of DMF and then admixed with 3.2 mg (17 μmol) of 1-(3-dimethylaminopropyl)-3-ethylcarbodiimide hydrochloride, 2.6 mg (17 μmol) of 1-hydroxy-1H-benzotriazole hydrate, 1.96 μl of N,N-diisopropylethylamine and with 5.9 mg (22.5 μmol) of commercially available 6-(2,5-dioxo-2,5-dihydro-1H-pyrrol-1-yl)hexanehydrazide. The mixture was stirred at RT overnight and then concentrated under high vacuum. The remaining residue was purified by means of preparative HPLC. Thus, 2.2 mg (36% of theory) of the title compound were obtained as a colourless foam.

HPLC (Method 5): Rₜ=1.7 min;

LC-MS (Method 1): Rₜ=0.88 min; MS (ESIpos): m/z=1094 (M+H)⁺.

### Intermediate 129

### N-(6-{2-[6-(2,5-dioxo-2,5-dihydro-1H-pyrrol-1-yl)hexanoyl]hydrazino}-6-oxohexyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

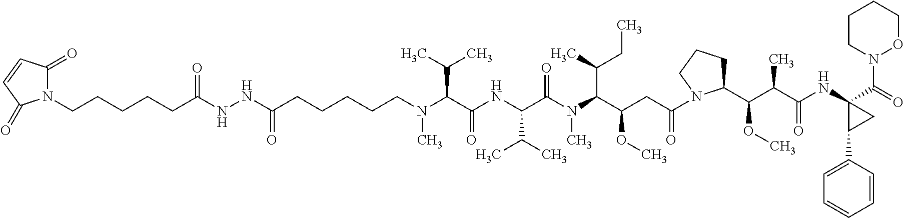 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCCCC(=O)NNC(=O)CCCCCN1C(=O)C=CC1=O)C(C)C]

4 mg (4.3 μmol) of N-(5-carboxypentyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 646 μl of DMF and then admixed with 2.5 mg (13 μmol) of 1-(3-dimethylaminopropyl)-3-ethylcarbodiimide hydrochloride, 2.0 mg (13 μmol) of 1-hydroxy-1H-benzotriazole hydrate, 2.25 μl of N,N-diisopropylethylamine and with 4.5 mg (17 μmol) of commercially available 6-(2,5-dioxo-2,5-dihydro-1H-pyrrol-1-yl)hexanehydrazide. The mixture was stirred at RT for 3 h and then concentrated under high vacuum. The remaining residue was purified by means of preparative HPLC. Thus, 1.9 mg (39% of theory) of the title compound were obtained as a colourless foam.

HPLC (Method 5): Rₜ=1.7 min;

LC-MS (Method 9): Rₜ=4.9 min; MS (ESIpos): m/z=1134 (M+H)⁺.

### Intermediate 130

### N-(4-{[(2R)-1-({5-[(2,5-dioxopyrrolidin-1-yl)oxy]-5-oxopentanoyl}amino)propan-2-yl]oxy}-4-oxobutyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

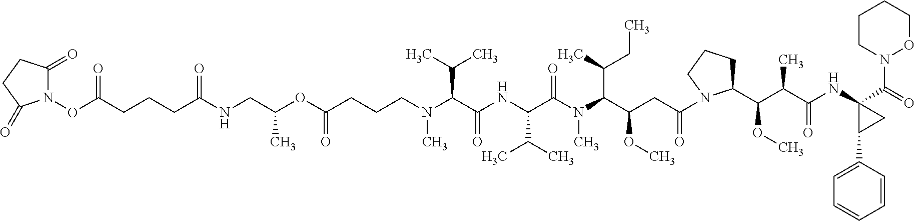 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)O[C@H](C)CNC(=O)CCCC(=O)ON1C(=O)CCC1=O)C(C)C]

10.5 mg (11.7 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 3.7 ml of dichloromethane and then admixed with 6.7 mg (35 μmol) of 1-(3-dimethylaminopropyl)-3-ethylcarbodiimide hydrochloride, 0.7 mg (5.8 μmol) of 4-dimethylaminopyridine and with 8.2 mg (47 μmol) of commercially available tert-butyl (2R)-2-hydroxypropyl carbamate. The mixture was stirred at RT overnight and then concentrated under high vacuum. The remaining residue was purified by means of preparative HPLC. Thus, 7.5 mg (61% of theory) of the Boc-protected intermediate were obtained as a colourless foam.

HPLC (Method 5): Rₜ=2.0 min;

LC-MS (Method 1): Rₜ=1.03 min; MS (ESIpos): m/z=1056 (M+H)⁺.

Subsequently, the Boc protecting group was detached with trifluoroacetic acid. 4.9 mg (0.005 mmol) of the deprotected crude product were then, without further purification, taken up in 1.8 ml of dichloromethane and admixed with 3.7 mg (0.011 mmol) of 1,1′-[(1,5-dioxopentane-1,5-diyl)bis(oxy)]dipyrrolidine-2,5-dione, 2.4 μl (0.014 mmol) of N,N-diisopropylethylamine and 0.6 mg (5 μmol) of 4-dimethylaminopyridine. The mixture was stirred at RT for 2 h and then concentrated under high vacuum. The remaining residue was purified by means of preparative HPLC. Thus, 0.77 mg (15% of theory) of the title compound were obtained as a colourless foam.

HPLC (Method 5): Rₜ=1.8 min;

LC-MS (Method 1): Rₜ=0.93 min; MS (ESIpos): m/z=1167 (M+H)⁺.

### Intermediate 131

### N-{4-[(1-{5[(2,5-dioxopyrrolidin-1-yl)oxy]-5-oxopentanoyl}piperidin-4-yl)oxy]-4-oxobutyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

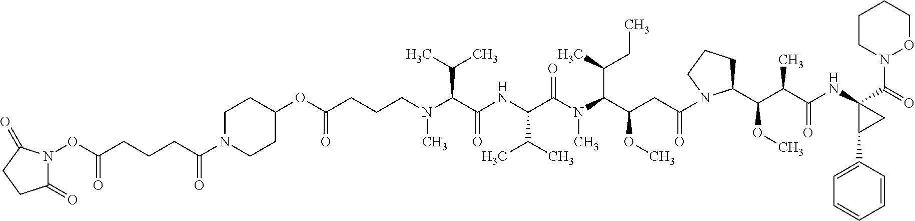 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)OC1CCN(C(=O)CCCC(=O)ON2C(=O)CCC2=O)CC1)C(C)C]

10 mg (11 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 2 ml of dichloromethane and then admixed with 4.3 mg (22 μmol) of 1-(3-dimethylaminopropyl)-3-ethylcarbodiimide hydrochloride, 0.88 mg (6 μmol) of 4-dimethylaminopyridine and with 5.2 mg (22 μmol) of commercially available benzyl 4-hydroxypiperidine-1-carboxylate. The mixture was stirred at RT overnight and then concentrated under high vacuum. The remaining residue was purified by means of preparative HPLC. Thus, 5 mg (40% of theory) of the Z-protected intermediate were obtained as a colourless foam.

HPLC (Method 5): Rₜ=2.1 min;

LC-MS (Method 1): Rₜ=1.04 min; MS (ESIpos): m/z=1116 (M+H)⁺.

Subsequently, the Z protecting group was detached by hydrogenolytic means in ethanol over palladium/activated carbon. 4.6 mg (0.005 mmol) of the deprotected crude product were then, without further purification, taken up in 1.8 ml of dichloromethane and admixed with 3.8 mg (0.012 mmol) of 1,1′-[(1,5-dioxopentane-1,5-diyl)bis(oxy)]dipyrrolidine-2,5-dione, 0.8 μl (0.005 mmol) of N,N-diisopropylethylamine and 0.6 mg (5 μmol) of 4-dimethylaminopyridine. The mixture was stirred at RT overnight and then concentrated under high vacuum. The remaining residue was purified by means of preparative HPLC. Thus, 0.96 mg (16% of theory) of the title compound were obtained as a colourless foam.

HPLC (Method 5): Rₜ=1.8 min;

LC-MS (Method 1): Rₜ=0.94 min; MS (ESIpos): m/z=1193 (M+H)⁺.

### Intermediate 132

### N-(4-{2-[6-(2,5-dioxo-2,5-dihydro-1H-pyrrol-1-yl)hexanoyl]hydrazinyl}-4-oxobutyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

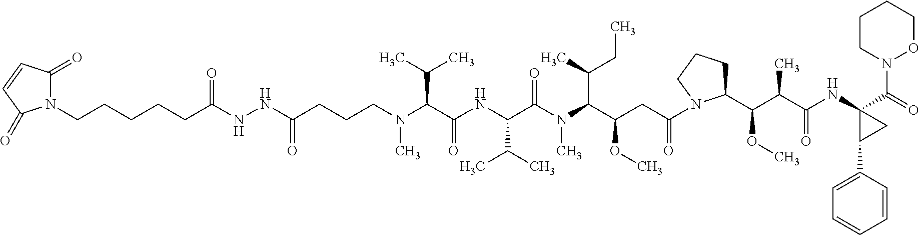 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)NNC(=O)CCCCCN1C(=O)C=CC1=O)C(C)C]

15 mg (16.7 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 2500 μl of DMF and then admixed with 9.6 mg (50 μmol) of 1-(3-dimethylaminopropyl)-3-ethylcarbodiimide hydrochloride, 7.6 mg (50 μmol) of 1-hydroxy-1H-benzotriazole hydrate, 5.8 μl of N,N-diisopropylethylamine and with 17.4 mg (67 μmol) of commercially available 6-(2,5-dioxo-2,5-dihydro-1H-pyrrol-1-yl)hexanehydrazide. The mixture was stirred at RT overnight and then concentrated under high vacuum. The remaining residue was purified by means of preparative HPLC. Thus, 11.2 mg (52% of theory) of the title compound were obtained as a colourless foam.

HPLC (Method 5): Rₜ=1.7 min;

LC-MS (Method 2): Rₜ=1.09 min; MS (ESIpos): m/z=1106 (M+H)⁺.

### Intermediate 133

### N-(4-{2-[6-(2,5-dioxo-2,5-dihydro-1H-pyrrol-1-yl)hexanoyl]hydrazinyl}-4-oxobutyl)-N-methyl-L-valyl-N-[(3R,4S,5S)-1-{(2S)-2-[(1R,2R)-3-{[(2S,3S)-1-(benzyloxy)-1-oxo-3-phenylbutan-2-yl]amino}-1-methoxy-2-methyl-3-oxopropyl]pyrrolidin-1-yl}-3-methoxy-5-methyl-1-oxoheptan-4-yl]-N-methyl-L-valinamide

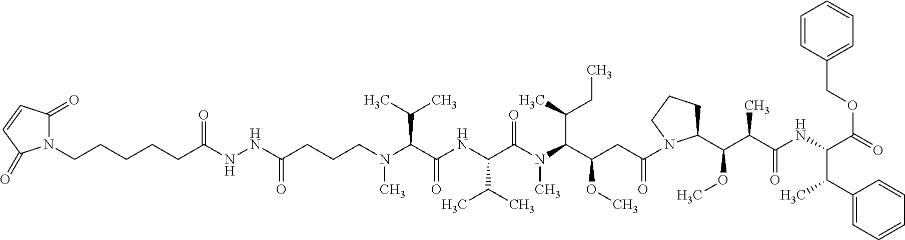 [SMILES: CC[C@H](C)[C@@H]([C@@H](CC(=O)N1CCC[C@H]1[C@H](OC)[C@@H](C)C(=O)N[C@H](C(=O)OCc1ccccc1)[C@@H](C)c1ccccc1)OC)N(C)C(=O)[C@@H](NC(=O)[C@H](C(C)C)N(C)CCCC(=O)NNC(=O)CCCCCN1C(=O)C=CC1=O)C(C)C]

5.8 mg (6.3 μmol) of N-(3-carboxypropyl)-N-methyl-L-valyl-N-[(3R,4S,5S)-1-{(2S)-2-[(1R,2R)-3-{[(2S,3S)-1-(benzyloxy)-1-oxo-3-phenylbutan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 943 μl of DMF and then admixed with 3.6 mg (19 μmol) of 1-(3-dimethylaminopropyl)-3-ethylcarbodiimide hydrochloride, 2.9 mg (19 μmol) of 1-hydroxy-1H-benzotriazole hydrate, 2.2 μl of N,N-diisopropylethylamine and with 6.6 mg (25 μmol) of commercially available 6-(2,5-dioxo-2,5-dihydro-1H-pyrrol-1-yl)hexanehydrazide. The mixture was stirred at RT overnight and then concentrated under high vacuum. The remaining residue was purified by means of preparative HPLC. Thus, 4.5 mg (64% of theory) of the title compound were obtained as a colourless foam.

HPLC (Method 5): Rₜ=2.0 min;

LC-MS (Method 1): Rₜ=1.03 min; MS (ESIpos): m/z=1129 (M+H)⁺.

### Intermediate 134

### N-[3-({[2-(2,5-dioxo-2,5-dihydro-1H-pyrrol-1-yl)ethyl]carbamoyl}amino)propyl]-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

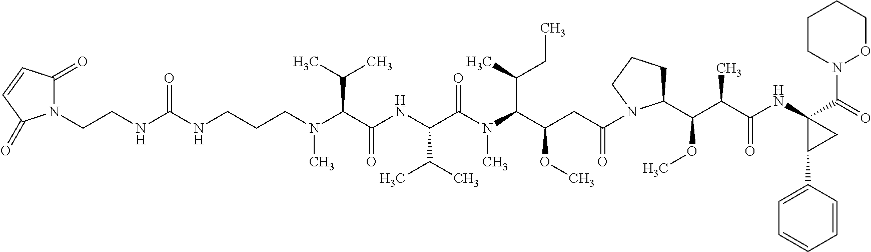 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCNC(=O)NCCN1C(=O)C=CC1=O)C(C)C]

First, 4-nitrophenyl 2-(2,5-dioxo-2,5-dihydro-1H-pyrrol-1-yl)ethyl carbamate was prepared under standard conditions, proceeding from commercially available 1-(2-aminoethyl)-1H-pyrrole-2,5-dione trifluoroacetate and 4-nitrophenyl chlorocarbonate.

5 mg (6 μmol) of N-(3-aminopropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 1000 μl of DMF and then admixed with 2 μl of N,N-diisopropylethylamine and with 2.2 mg (9 μmol) of 4-nitrophenyl 2-(2,5-dioxo-2,5-dihydro-1H-pyrrol-1-yl)ethyl carbamate. The mixture was stirred at RT for 1 h and then concentrated under high vacuum. The remaining residue was purified by means of preparative HPLC. Thus, 1.6 mg (23% of theory) of the title compound were obtained as a colourless foam.

HPLC (Method 5): Rₜ=1.7 min;

LC-MS (Method 2): Rₜ=1.09 min; MS (ESIpos): m/z=1036 (M+H)⁺.

### Intermediate 135

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

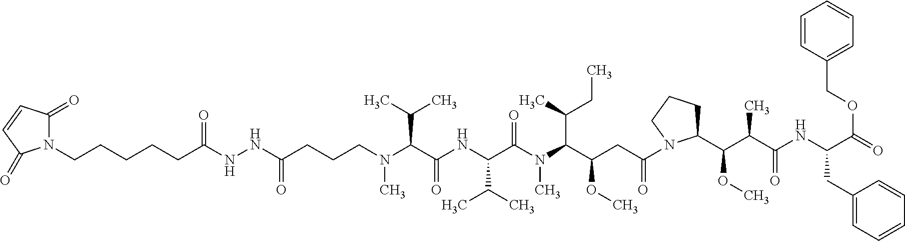 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccccc1)OC)N(C)C(=O)[C@@H](NC(=O)[C@H](C(C)C)N(C)CCCC(=O)NNC(=O)CCCCCN1C(=O)C=CC1=O)C(C)C]

10 mg (11 μmol) of N-(3-carboxypropyl)-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 4000 μl of DMF and then admixed with 6.3 mg (33 μmol) of 1-(3-dimethylaminopropyl)-3-ethylcarbodiimide hydrochloride, 4.5 mg (33 μmol) of 1-hydroxy-1H-benzotriazole hydrate, 5.7 μl of N,N-diisopropylethylamine and with 11.5 mg (44 μmol) of commercially available 6-(2,5-dioxo-2,5-dihydro-1H-pyrrol-1-yl)hexanehydrazide. The mixture was stirred at RT overnight and then concentrated under high vacuum. The remaining residue was purified by means of preparative HPLC. Thus, 2.6 mg (14% of theory) of the title compound were obtained as a colourless foam.

HPLC (Method 6): Rₜ=2.1 min;

LC-MS (Method 1): Rₜ=1.01 min; MS (ESIpos): m/z=1115 (M+H)⁺.

### Intermediate 136

### N-(4-{4-[4-(2,5-dioxo-2,5-dihydro-1H-pyrrol-1-yl)butanoyl]piperazin-1-yl}-4-oxobutyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

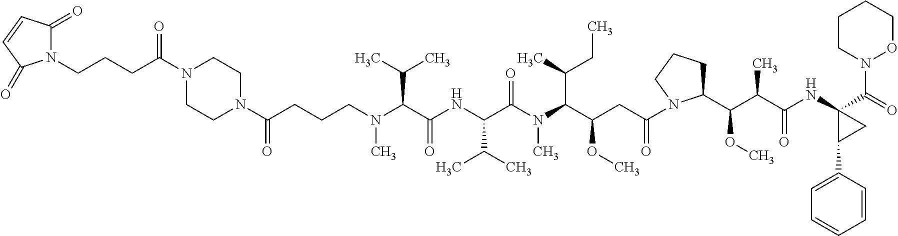 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)N1CCN(C(=O)CCCN2C(=O)C=CC2=O)CC1)C(C)C]

First, 1-[4-oxo-4-(piperazin-1-yl)butyl]-1H-pyrrole-2,5-dione trifluoroacetate was prepared under standard conditions, proceeding from tert-butyl piperazine-1-carboxylate and 4-(2,5-dioxo-2,5-dihydro-1H-pyrrol-1-yl)butanoic acid over 2 stages.

5 mg (5.6 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 1000 μl of DMF and then admixed with 2.1 mg (11 μmol) of 1-(3-dimethylaminopropyl)-3-ethylcarbodiimide hydrochloride, 1.7 mg (11 μmol) of 1-hydroxy-1H-benzotriazole hydrate, 2 μl of N,N-diisopropylethylamine and with 3.5 mg (5.6 μmol) of 1-[4-oxo-4-(piperazin-1-yl)butyl]-1H-pyrrole-2,5-dione trifluoroacetate. The mixture was stirred at RT overnight. Then 2.1 mg (5.6 μmol) of O-(7-azabenzotriazol-1-yl)-N,N,N′,N′-tetramethyluronium hexafluorophosphate were added and the reaction mixture was stirred at RT for a further 3 h. Subsequently, the solvent was removed under reduced pressure and the remaining residue was purified by means of preparative HPLC. The corresponding fractions were concentrated and, by lyophilization from water, 0.6 mg (10% of theory) of the title compound was obtained as a colourless foam.

HPLC (Method 6): Rₜ=1.9 min;

LC-MS (Method 1): Rₜ=0.9 min; MS (ESIpos): m/z=1132 (M+H)⁺.

### Intermediate 137

### N-(4-{2-[6-(2,5-dioxo-2,5-dihydro-1H-pyrrol-1-yl)hexanoyl]-1-methylhydrazino}-4-oxobutyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

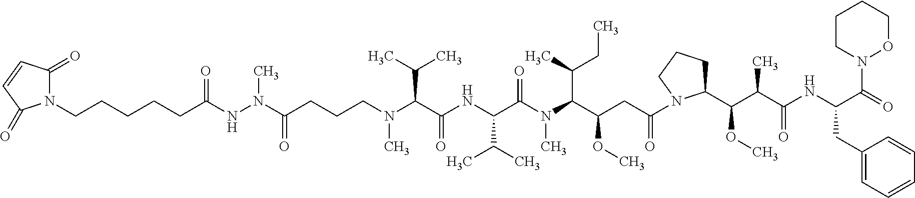 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCC(=O)N(C)NC(=O)CCCCCN1C(=O)C=CC1=O)C(C)C]

First, 6-(2,5-dioxo-2,5-dihydro-1H-pyrrol-1-yl)-N′-methylhexanehydrazide trifluoroacetate was prepared under standard conditions, proceeding from commercially available 6-(2,5-dioxo-2,5-dihydro-1H-pyrrol-1-yl)hexanoic acid and tert-butyl 1-methylhydrazinecarboxylate over 2 stages.

6.9 mg (8 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide were dissolved in 2540 μl of DMF and then admixed with 3.6 mg (9 μmol) of O-(7-azabenzotriazol-1-yl)-N,N,N′,N′-tetramethyluronium hexafluorophosphate, 3 μl of N,N-diisopropylethylamine and with 4.1 mg (12 μmol) of 6-(2,5-dioxo-2,5-dihydro-1H-pyrrol-1-yl)-N′-methylhexanehydrazide trifluoroacetate. The mixture was stirred at RT overnight. Subsequently, the solvent was removed under reduced pressure and the remaining residue was purified by means of preparative HPLC. Thus, 3.9 mg (45% of theory) of the title compound were obtained as a colourless foam.

HPLC (Method 5): Rₜ=1.8 min;

LC-MS (Method 1): Rₜ=0.93 min; MS (ESIpos): m/z=1108 (M+H)⁺.

### Intermediate 138

### N-{4-[(2-{[4-(2,5-dioxo-2,5-dihydro-1H-pyrrol-1-yl)butanoyl](methyl)amino}ethyl)(methyl)amino]-4-oxobutyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

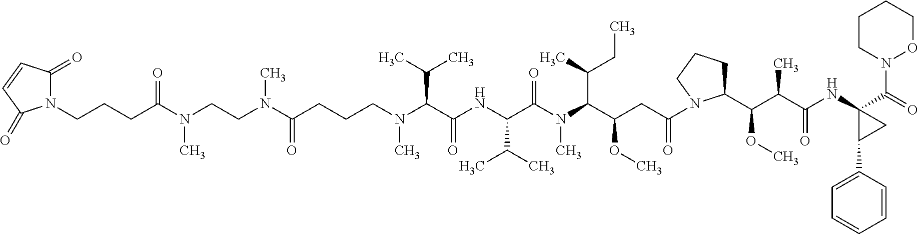 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)N(C)CCN(C)C(=O)CCCN1C(=O)C=CC1=O)C(C)C]

Proceeding from tert-butylmethyl 2-(methylamino)ethyl carbamate and 4-(2,5-dioxo-2,5-dihydro-1H-pyrrol-1-yl)butanoic acid, over 2 stages, 4-(2,5-dioxo-2,5-dihydro-1H-pyrrol-1-yl)-N-methyl-N-[2-(methylamino)ethyl]butanamide trifluoroacetate was first prepared by.

6.6 mg (7.3 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 2000 μl of DMF and then admixed with 5.6 mg (14.7 μmol) of O-(7-azabenzotriazol-1-yl)-N,N,N′,N′-tetramethyluronium hexafluorophosphate, 2.6 μl of N,N-diisopropylethylamine and with 4.1 mg (9 μmol) of 4-(2,5-dioxo-2,5-dihydro-1H-pyrrol-1-yl)-N-methyl-N-[2-(methylamino)ethyl]butanamide trifluoroacetate. After stirring at RT for 3 h, the same amounts of HATU and N,N-diisopropylethylamine were added once again, and the reaction mixture was then stirred at RT overnight. Subsequently, the solvent was removed under reduced pressure and the remaining residue was purified by means of preparative HPLC. Thus, 4 mg (44% of theory) of the title compound were obtained as a colourless foam.

HPLC (Method 6): Rₜ=2.0 min;

LC-MS (Method 1): Rₜ=0.91 min; MS (ESIpos): m/z=1134 (M+H)⁺.

### Intermediate 139

### (2R,3S)-3-amino-4-{2-[6-(2,5-dioxo-2,5-dihydro-1H-pyrrol-1-yl)hexanoyl]hydrazino}-4-oxobutan-2-yl (3R,4S,7S,10S)-4-[(2S)-butan-2-yl]-7,10-diisopropyl-3-(2-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-2-oxoethyl)-5,11-dimethyl-6,9-dioxo-2-oxa-5,8,11-triazapentadecan-15-oate

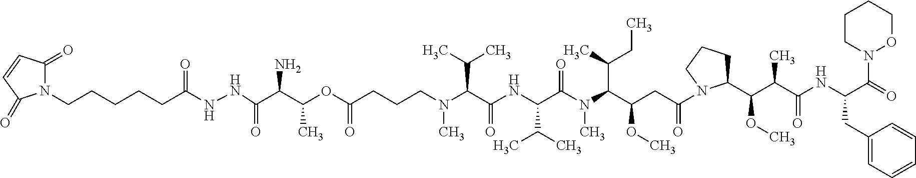 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCC(=O)O[C@H](C)[C@H](N)C(=O)NNC(=O)CCCCCN1C(=O)C=CC1=O)C(C)C]

13 mg (14.7 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide were dissolved in 10 ml of dichloromethane and then admixed with 8.4 mg (44 μmol) of 1-(3-dimethylaminopropyl)-3-ethylcarbodiimide hydrochloride, 5.4 mg (44 μmol) of 4-dimethylaminopyridine and with 9 mg (29.3 μmol) of commercially available benzyl N-(tert-butoxycarbonyl)-L-threoninate. The mixture was stirred at RT for 5 h. Subsequently, the reaction mixture was twice extracted by shaking with water and the organic phase was dried over sodium sulphate and concentrated under reduced pressure. The remaining residue was purified by means of preparative HPLC. After lyophilization from dioxane/water, 14 mg (81% of theory) of the protected intermediate were obtained as a colourless foam.

HPLC (Method 12): Rₜ=2 3 min;

LC-MS (Method 1): Rₜ=1.13 min; MS (ESIpos): m/z=1178 (M+H)⁺.

Subsequently, the Z protecting group was detached by hydrogenolytic means in methanol over 10% palladium/activated carbon. 9.5 mg (0.0087 mmol) of the deprotected crude product were then, without further purification, taken up in 5 ml of DMF, and admixed 5 mg (26.2 μmol) of 1-(3-dimethylaminopropyl)-3-ethylcarbodiimide hydrochloride, 4 mg (26.2 μmol) of 1-hydroxy-1H-benzotriazole hydrate, 54.6 μl of N,N-diisopropylethylamine and with 9.1 mg (34.9 μmol) of commercially available 6-(2,5-dioxo-2,5-dihydro-1H-pyrrol-1-yl)hexanehydrazide. The mixture was stirred at RT for 1 h and then concentrated under high vacuum. The remaining residue was purified by means of preparative HPLC. After lyophilization from dioxane, 9.5 mg (84% of theory) of the Boc-protected intermediate were obtained.

HPLC (Method 12): Rₜ=2.1 min;

LC-MS (Method 1): Rₜ=0.97 min; MS (ESIpos): m/z=1295 (M+H)⁺.

Subsequently, 9.5 mg (7.3 μmol) were deprotected with 0.5 ml of trifluoroacetic acid in 2 ml of dichloromethane of the Boc-protected intermediate and, after lyophilization from dioxane, 9 mg (82% of theory) of the title compound were obtained as a colourless foam.

HPLC (Method 12): Rₜ=2.1 min;

LC-MS (Method 1): Rₜ=0.84 min; MS (ESIpos): m/z=1195 (M+H)⁺.

### Intermediate 140

### N-(4-{2-[6-(2,5-dioxo-2,5-dihydro-1H-pyrrol-1-yl)hexanoyl]-1-methylhydrazino}-4-oxobutyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

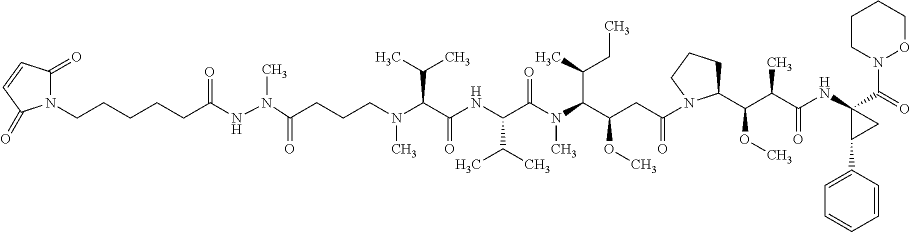 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)N(C)NC(=O)CCCCCN1C(=O)C=CC1=O)C(C)C]

4.1 mg (12 μmol) of 6-(2,5-dioxo-2,5-dihydro-1H-pyrrol-1-yl)-N-methylhexanehydrazide trifluoroacetate (Intermediate 22) were dissolved together with 6.9 mg (8 μmol) of the compound from Intermediate 61 in 2.5 ml of DMF and then admixed with 3.5 mg (9 μmol) of O-(7-azabenzotriazol-1-yl)-N,N,N′,N′-tetramethyluronium hexafluorophosphate and 3 μl of N,N-diisopropylethylamine. The mixture was stirred at RT overnight and then concentrated under high vacuum. The remaining residue was purified by means of preparative HPLC. After lyophilization from dioxane, 2.6 mg (30% of theory) of the title compound were obtained.

HPLC (Method 5): Rₜ=1.8 min;

LC-MS (Method 1): Rₜ=0.90 and 0.91 min; MS (ESIpos): m/z=1120 (M+H)⁺.

### Intermediate 141

### N-[4-({1-[4-(2,5-dioxo-2,5-dihydro-1H-pyrrol-1-yl)butanoyl]piperidin-4-yl}oxy)-4-oxobutyl]-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

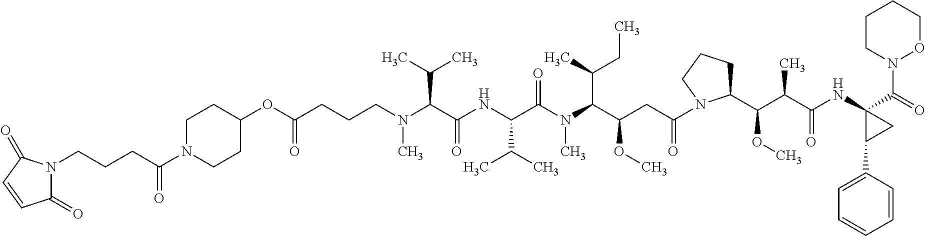 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)OC1CCN(C(=O)CCCN2C(=O)C=CC2=O)CC1)C(C)C]

44 mg (49 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide were dissolved in 2 ml of dichloromethane and then admixed with 18.8 mg (98 μmol) of 1-(3-dimethylaminopropyl)-3-ethylcarbodiimide hydrochloride, 3.8 mg (24 μmol) of 4-dimethylaminopyridine and with 23 mg (98 μmol) of commercially available benzyl 4-hydroxypiperidine-1-carboxylate. The mixture was stirred at RT overnight and then concentrated under high vacuum. The remaining residue was purified by means of preparative HPLC. Thus, 22 mg (40% of theory) of the Z-protected intermediate were obtained as a colourless foam.

HPLC (Method 5): Rₜ=2.1 min;

LC-MS (Method 1): Rₜ=1.04 min; MS (ESIpos): m/z=1116 (M+H)⁺.

Subsequently, the Z protecting group was detached by hydrogenolytic means in ethanol over palladium/activated carbon.

19 mg (19 μmol) of the deprotected crude product were then, without further purification, taken up in 4 ml of DMF and admixed with 7 mg (39 μmol) of 4-(2,5-dioxo-2,5-dihydro-1H-pyrrol-1-yl)butanoic acid, 11 mg (29 μmol) of O-(7-azabenzotriazol-1-yl)-N,N,N′,N′-tetramethyluronium hexafluorophosphate and 5 μl of N,N-diisopropylethylamine. The mixture was stirred at RT for 1 h and then concentrated under high vacuum. The remaining residue was purified by means of preparative HPLC. After lyophilization from dioxane, 7.5 mg (34% of theory) of the title compound were obtained.

HPLC (Method 5): Rₜ=1.8 min;

LC-MS (Method 1): Rₜ=0.94 min; MS (ESIpos): m/z=1147 (M+H)⁺.

### Intermediate 142

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-(benzyloxy)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

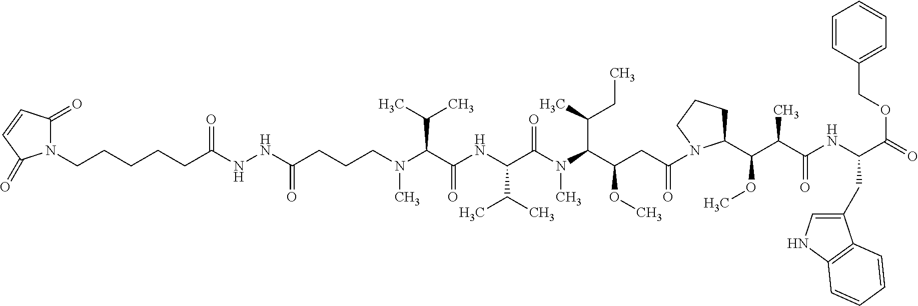 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)OCc1ccccc1)OC)N(C)C(=O)[C@@H](NC(=O)[C@H](C(C)C)N(C)CCCC(=O)NNC(=O)CCCCCN1C(=O)C=CC1=O)C(C)C]

9 mg (9.5 μmol) of N-(3-carboxypropyl)-N-methyl-L-valyl-N-[(3R,4S,5S)-1-{(2S)-2-[(1R,2R)-3-{[(2S)-1-(benzyloxy)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 72) were dissolved in 1000 μl of DMF and then admixed with 10 mg (38 μmol) of commercially available 6-(2,5-dioxo-2,5-dihydro-1H-pyrrol-1-yl)hexanehydrazide, 7.2 mg (19 μmol) of O-(7-azabenzotriazol-1-yl)-N,N,N′,N′-tetramethyluronium hexafluorophosphate and 8 μl of N,N-diisopropylethylamine, and the reaction mixture was stirred at RT for 1 h. Subsequently, the solvent was removed under reduced pressure and the remaining residue was purified by means of preparative HPLC. The corresponding fractions were concentrated and, by lyophilization, 6.4 mg (58% of theory) of the title compound were obtained as a colourless foam.

HPLC (Method 5): Rₜ=1.9 min;

LC-MS (Method 1): Rₜ=0.99 min; MS (ESIpos): m/z=1154 (M+H)⁺.

### Intermediate 143

### N-(4-{2-[4-(2,5-dioxo-2,5-dihydro-1H-pyrrol-1-yl)-2,2-dimethylbutanoyl]hydrazino}-4-oxobutyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

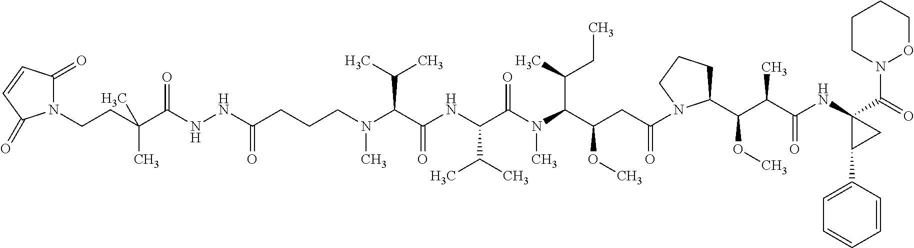 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)NNC(=O)C(C)(C)CCN1C(=O)C=CC1=O)C(C)C]

6 mg (6.7 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide (Intermediate 61) were reacted with 3 mg (8.7 μmol) of 4-(2,5-dioxo-2,5-dihydro-1H-pyrrol-1-yl)-2,2-dimethylbutanehydrazide trifluoroacetate in analogy to Intermediate 142 to give 2 mg (27% of theory) of the title compound.

HPLC (Method 12): Rₜ=2.1 min;

LC-MS (Method 3): Rₜ=1.92 min; MS (ESIpos): m/z=1106 (M+H)⁺.

### Intermediate 144

### N-(4-(2-[4-(2,5-dioxo-2,5-dihydro-1H-pyrrol-1-yl)-2,2-dimethylbutanoyl]hydrazinol-4-oxobutyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

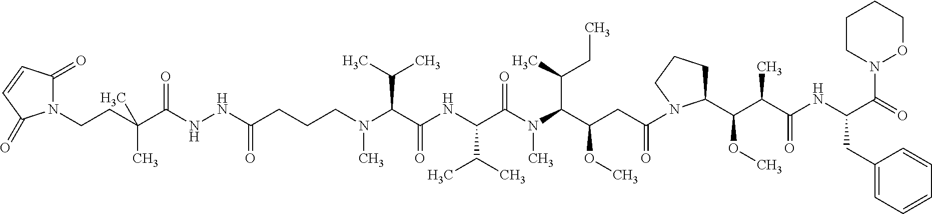 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1CCCCO1)OC)N(C)C(=O)[C@@H](NC(=O)[C@H](C(C)C)N(C)CCCC(=O)NNC(=O)C(C)(C)CCN1C(=O)C=CC1=O)C(C)C]

To a solution of 5 mg (5.6 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide in 1 ml of DMF were added 7.65 mg (22.5 μmol) of 4-(2,5-dioxo-2,5-dihydro-1H-pyrrol-1-yl)-2,2-dimethylbutanehydrazide trifluoroacetate, 3.2 mg (16.9 μmol) of EDC, 1.96 μl (11.3 μmol) of diisopropylethylamine and 2.6 mg (16.9 μmol) of HOBT. The reaction mixture was stirred at RT for 3 h. Subsequently, a further 0.95 mg (2.8 μmol) of 4-(2,5-dioxo-2,5-dihydro-1H-pyrrol-1-yl)-2,2-dimethylbutanehydrazide trifluoroacetate was added. After stirring overnight, the reaction mixture was concentrated and purified by preparative HPLC. 3.5 mg (85% purity, 48% of theory) of the title compound were obtained.

LC-MS (Method 3): Rₜ=1.86 min; m/z=1094 (M+H)⁺.

### Intermediate 145

### N-[3-(2,5-dioxo-2,5-dihydro-1H-pyrrol-1-yl)propyl]-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenyl cyclopropyl]amino}-3-oxopropyl]pyrrolidin-1-yl}-5-methyl-1-oxoheptan-4-yl]-N-methyl-L-valinamide

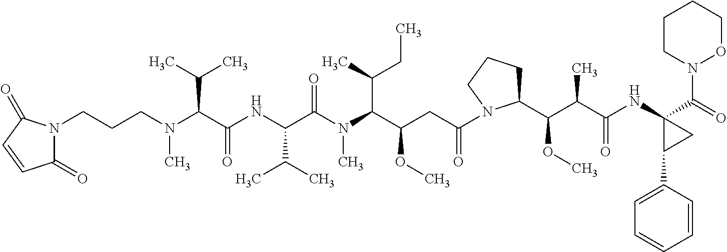 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCN1C(=O)C=CC1=O)C(C)C]

12 mg (14 μmol) of N-(3-aminopropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide (Intermediate 66) were taken up in 750 μl of dioxane and admixed with 1.5 ml of saturated sodium hydrogencarbonate solution and then with 3.2 mg (21 μmol) of methyl 2,5-dioxo-2,5-dihydro-1H-pyrrole-1-carboxylate. The reaction mixture was stirred at RT for 1 h and then concentrated under reduced pressure. The remaining residue was purified by means of preparative HPLC. After lyophilization, 4.2 mg (32% of theory) of the title compound were obtained.

HPLC (Method 5): Rₜ=1.7 min;

LC-MS (Method 1): Rₜ=0.94 min; MS (ESIpos): m/z=950 (M+H)⁺.

### Intermediate 146

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-({(2S)-1-[benzyl(methyl)amino]-1-oxo-3-phenylpropan-2-yl}amino)-1-methoxy-2-methyl-3-oxopropyl]pyrrolidin-1-yl}-3-methoxy-5-methyl-1-oxoheptan-4-yl]-N-methyl-L-valinamide

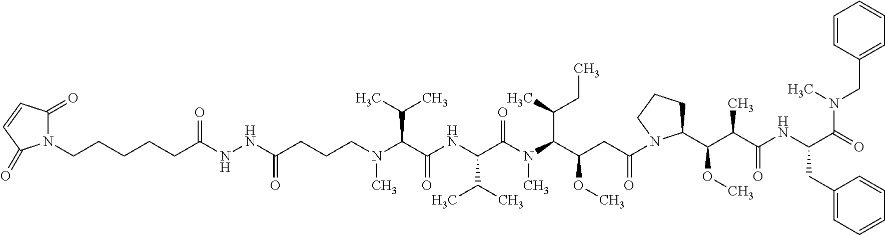 [SMILES: CC[C@H](C)[C@@H]([C@@H](CC(=O)N1CCC[C@H]1[C@H](OC)[C@@H](C)C(=O)N[C@@H](Cc1ccccc1)C(=O)N(C)Cc1ccccc1)OC)N(C)C(=O)[C@@H](NC(=O)[C@H](C(C)C)N(C)CCCC(=O)NNC(=O)CCCCCN1C(=O)C=CC1=O)C(C)C]

9 mg (9.8 μmol) of N-(3-carboxypropyl)-N-methyl-L-valyl-N-[(3R,4S,5S)-1-{(2S)-2-[(1R,2R)-3-({(2S)-1-[benzyl(methyl)amino]-1-oxo-3-phenylpropan-2-yl}amino)-1-methoxy-2-methyl-3-oxopropyl]pyrrolidin-1-yl}-3-methoxy-5-methyl-1-oxoheptan-4-yl]-N-methyl-L-valinamide (Intermediate 73) were reacted in analogy to Intermediate 133 with 10 mg (39 μmol) of 6-(2,5-dioxo-2,5-dihydro-1H-pyrrol-1-yl)hexanehydrazide to give 1.8 mg (15% of theory) of the title compound.

HPLC (Method 12): Rₜ=2.2 min;

LC-MS (Method 9): Rₜ=5.11 min; MS (ESIpos): m/z=1128 (M+H)⁺.

### Intermediate 147

### N-{4-[(2,5-dioxopyrrolidin-1-yl)oxy]-4-oxobutyl}-N-methyl-L-valyl-N-[(3R,4S,5S)-1-{(2S)-2-[(1R,2R)-3-{[(2S,3S)-1-(benzyloxy)-1-oxo-3-phenylbutan-2-yl]amino}-1-methoxy-2-methyl-3-oxopropyl]pyrrolidin-1-yl}-3-methoxy-5-methyl-1-oxoheptan-4-yl]-N-methyl-L-valinamide

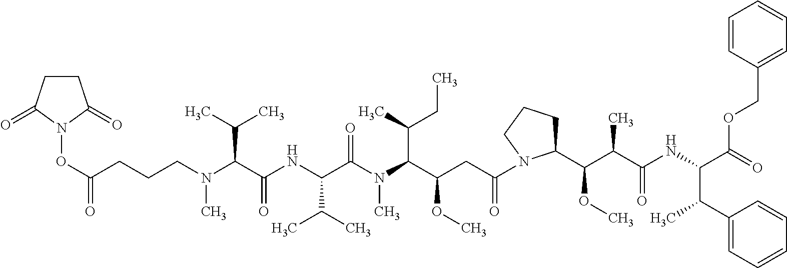 [SMILES: CC[C@H](C)[C@@H]([C@@H](CC(=O)N1CCC[C@H]1[C@H](OC)[C@@H](C)C(=O)N[C@H](C(=O)OCc1ccccc1)[C@@H](C)c1ccccc1)OC)N(C)C(=O)[C@@H](NC(=O)[C@H](C(C)C)N(C)CCCC(=O)ON1C(=O)CCC1=O)C(C)C]

16 mg (17 μmol) of N-(3-carboxypropyl)-N-methyl-L-valyl-N-[(3R,4S,5S)-1-{(2S)-2-[(1R,2R)-3-{[(2S,3S)-1-(benzyloxy)-1-oxo-3-phenylbutan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 70) were dissolved in 2 ml of dichloromethane and admixed with 2.6 mg (23 mmol) of 1-hydroxypyrrolidine-2,5-dione and then with 4 mg (21 μmol) of 1-(3-dimethylaminopropyl)-3-ethylcarbodiimide hydrochloride. After stirring at RT for 2 h, the same amounts of 1-hydroxypyrrolidine-2,5-dione and 1-(3-dimethylaminopropyl)-3-ethylcarbodiimide hydrochloride were added once again. Then stirring at RT overnight, the reaction mixture was concentrated under reduced pressure. The remaining residue was purified by means of preparative HPLC. After lyophilization, 10 mg (56% of theory) of the title compound were obtained.

HPLC (Method 5): Rₜ=2.0 min;

### Intermediate 148

### N-{4-[(2-{[4-(2,5-dioxo-2,5-dihydro-1H-pyrrol-1-yl)butanoyl](methyl)amino}ethyl)amino]-4-oxobutyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

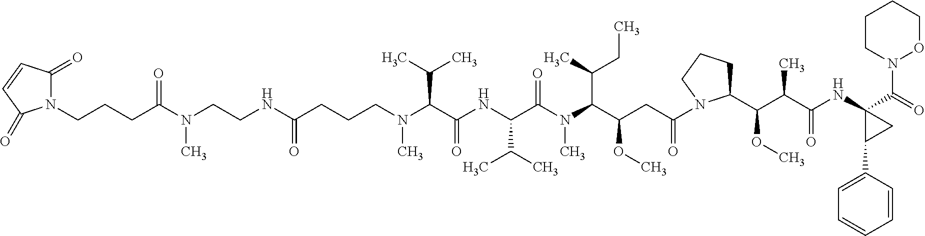 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)NCCN(C)C(=O)CCCN1C(=O)C=CC1=O)C(C)C]

6 mg (7 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide (Intermediate 61) were combined with 2.8 mg (8 μmol) of N-(2-aminoethyl)-4-(2,5-dioxo-2,5-dihydro-1H-pyrrol-1-yl)-N-methylbutanamide trifluoroacetate, 10.1 mg (27 μmol) of O-(7-azabenzotriazol-1-yl)-N,N,N′,N′-tetramethyluronium hexafluorophosphate and 5 μl of N,N-diisopropylethylamine in 2 ml of DMF and stirred at RT overnight. Then another 5 mg (23.5 μmol) of O-(7-azabenzotriazol-1-yl)-N,N,N′,N′-tetramethyluronium hexafluorophosphate and 3 μl of N,N-diisopropylethylamine were added. After stirring at RT for a further 5 h, the solvent was removed under reduced pressure and the remaining residue was purified by means of preparative HPLC. The corresponding fractions were concentrated and, by lyophilization from dioxane, 1.3 mg (15% of theory) of the title compound were obtained.

HPLC (Method 12): Rₜ=2.1 min;

LC-MS (Method 2): Rₜ=1.21 min; MS (ESIpos): m/z=1120 (M+H)⁺.

### Intermediate 149

### N-{4-[(2-{[4-(2,5-dioxo-2,5-dihydro-1H-pyrrol-1-yl)butanoyl]amino}ethyl)(methyl)amino]-4-oxobutyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

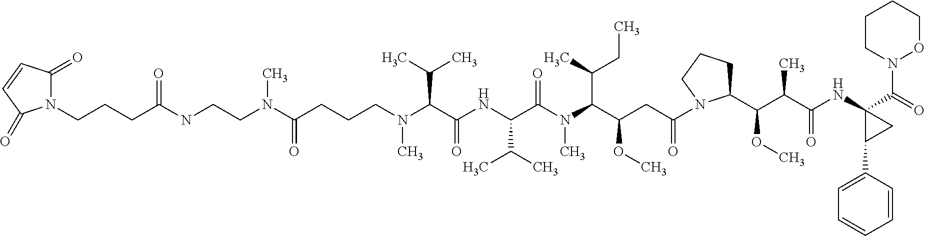 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)N(C)CCNC(=O)CCCN1C(=O)C=CC1=O)C(C)C]

6 mg (7 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide (Intermediate 61) were combined with 3.1 mg (9 μmol) of 4-(2,5-dioxo-2,5-dihydro-1H-pyrrol-1-yl)-N-[2-(methylamino)ethyl]butanamide trifluoroacetate, 10.1 mg (27 μmol) of O-(7-azabenzotriazol-1-yl)-N,N,N′,N′-tetramethyluronium hexafluorophosphate and 5 μl of N,N-diisopropylethylamine in 2 ml of DMF, and the mixture was stirred at RT for 4 h. Then the solvent was removed under reduced pressure and the remaining residue was purified by means of preparative HPLC. The corresponding fractions were concentrated and, by lyophilization from dioxane, 1 mg (13.4% of theory) of the title compound were obtained.

HPLC (Method 12): Rₜ=2.1 min;

LC-MS (Method 1): Rₜ=0.89 min; MS (ESIpos): m/z=1121 (M+H)⁺.

### Intermediate 150

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-oxo-3-{[(1S,2R)-2-phenyl-1-(propylcarbamoyl)cyclopropyl]amino}propyl]pyrrolidin-1-yl}-5-methyl-1-oxoheptan-4-yl]-N-methyl-L-valinamide

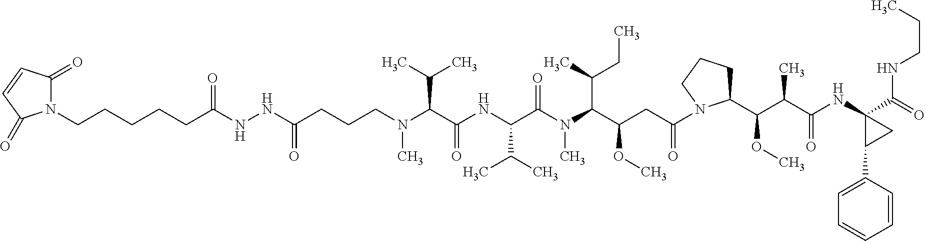 [SMILES: CCCNC(=O)[C@]1(NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)CCCC(=O)NNC(=O)CCCCCN2C(=O)C=CC2=O)C(C)C)C[C@@H]1c1ccccc1]

7.9 mg (9 μmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(1S,2R)-2-phenyl-1-(propylcarbamoyl)cyclopropyl]amino}propyl]pyrrolidin-1-yl}-5-methyl-1-oxoheptan-4-yl]-N-methyl-L-valinamide were dissolved in 3 ml of DMF and then admixed with 10.4 mg (54 μmol) of 1-(3-dimethylaminopropyl)-3-ethylcarbodiimide hydrochloride, 8.3 mg (54 μmol) of 1-hydroxy-1H-benzotriazole hydrate, 9 μl of N,N-diisopropylethylamine and with 9.5 mg (36 μmol) of commercially available 6-(2,5-dioxo-2,5-dihydro-1H-pyrrol-1-yl)hexanehydrazide. The mixture was stirred at RT overnight and then concentrated under high vacuum. The remaining residue was purified by means of preparative HPLC. Thus, 4.3 mg (22% of theory) of the title compound were obtained as a colourless foam.

HPLC (Method 6): Rₜ=1.9 min;

LC-MS (Method 9): Rₜ=4.93 min; MS (ESIpos): m/z=1078 (M+H)⁺.

### Intermediate 151

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(1S,2R)-1-carbamoyl-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

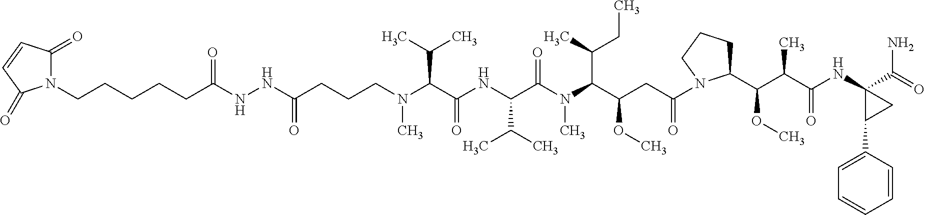 [SMILES: CC[C@H](C)[C@@H]([C@@H](CC(=O)N1CCC[C@H]1[C@H](OC)[C@@H](C)C(=O)N[C@@]1(C(N)=O)C[C@@H]1c1ccccc1)OC)N(C)C(=O)[C@@H](NC(=O)[C@H](C(C)C)N(C)CCCC(=O)NNC(=O)CCCCCN1C(=O)C=CC1=O)C(C)C]

The compound was prepared analogously to Intermediate 150, proceeding from the compound in Intermediate 81.

HPLC (Method 5): Rₜ=1.7 min;

LC-MS (Method 1): Rₜ=0.87 min; MS (ESIpos): m/z=1036 (M+H)⁺.

### Intermediate 152

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(1S,2R)-1-(ethoxycarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

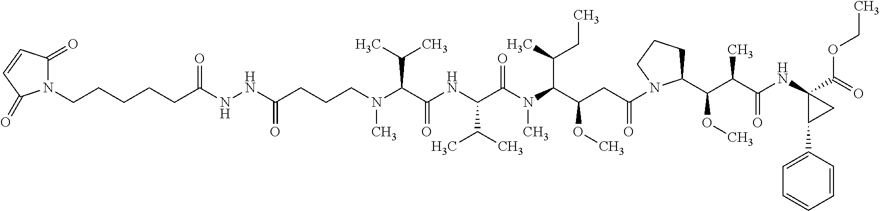 [SMILES: CCOC(=O)[C@]1(NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)CCCC(=O)NNC(=O)CCCCCN2C(=O)C=CC2=O)C(C)C)C[C@@H]1c1ccccc1]

10 mg (12 μmol) of N-(3-carboxypropyl)-N-methyl-L-valyl-N-[(3R,4S,5S)-1-{(2S)-2-[(1R,2R)-3-{[(1S,2R)-1-(ethoxycarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide were dissolved in 3 ml of DMF and then admixed with 8.9 mg (23 μmol) of O-(7-azabenzotriazol-1-yl)-N,N,N′,N′-tetramethyluronium hexafluorophosphate, 10 μl of N,N-diisopropylethylamine and with 12 mg (47 μmol) of commercially available 6-(2,5-dioxo-2,5-dihydro-1H-pyrrol-1-yl)hexanehydrazide. The mixture was stirred at RT for 1 h. This was followed by concentration under high vacuum and purification of the remaining residue by means of preparative HPLC. Thus, 5.8 mg (37% of theory) of the title compound were obtained as a colourless foam.

HPLC (Method 6): Rₜ=2.0 min;

LC-MS (Method 9): Rₜ=4.99 min; MS (ESIpos): m/z=1066 (M+H)⁺.

### Intermediate 153

### N-[1-(2,5-dioxo-2,5-dihydro-1H-pyrrol-1-yl)-12,15-dioxo-3,6,9-trioxa-13,14-diazaoctadecan-18-yl]-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide

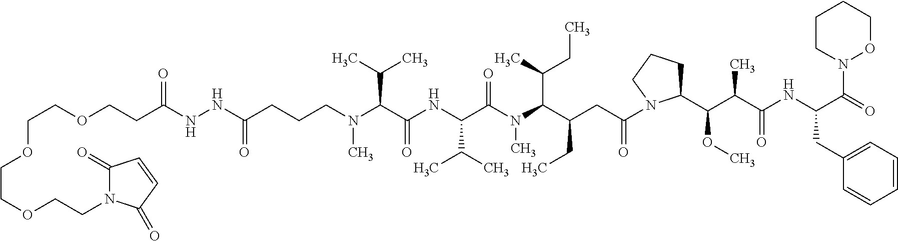 [SMILES: CC[C@H](CC(=O)N1CCC[C@H]1[C@H](OC)[C@@H](C)C(=O)N[C@@H](Cc1ccccc1)C(=O)N1CCCCO1)[C@H]([C@@H](C)CC)N(C)C(=O)[C@@H](NC(=O)[C@H](C(C)C)N(C)CCCC(=O)NNC(=O)CCOCCOCCOCCN1C(=O)C=CC1=O)C(C)C]

To a solution of 5 mg (5.6 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(2S)-1-(1,2-oxazinan-2-yl)-1-oxo-3-phenylpropan-2-yl]amino}-3-oxopropyl]pyrrolidin-1-yl}-5-methyl-1-oxoheptan-4-yl]-N-methyl-L-valinamide in 1 ml of DMF were added 9.7 mg (22.5 μmol) of 3-(2-{2-[2-(2,5-dioxo-2,5-dihydro-1H-pyrrol-1-yl)ethoxy]ethoxy}ethoxy)propanehydrazide trifluoroacetate, 3.2 mg (16.9 μmol) of EDC, 1.96 μl (11.3 μmol) of N,N-diisopropylethylamine and 2.6 mg (16.9 μmol) of HOBT. The reaction mixture was stirred at RT for 3 h. Subsequently, a further 1.2 mg (2.8 μmol) of 3-(2-{2-[2-(2,5-dioxo-2,5-dihydro-1H-pyrrol-1-yl)ethoxy]ethoxy}ethoxy)propanehydrazide trifluoroacetate were added. The reaction mixture was stirred at RT overnight and then purified by preparative HPLC.

3.6 mg (51% of theory) of the title compound were obtained.

LC-MS (Method 1): Rₜ=0.90 min; m/z=1185 (M+H)⁺.

### Intermediate 154

### (2R,3S)-3-amino-4-{2-[6-(2,5-dioxo-2,5-dihydro-1H-pyrrol-1-yl)hexanoyl]hydrazino}-4-oxobutan-2-yl (3R,4S,7S,10S)-4-[(2S)-butan-2-yl]-7,10-diisopropyl-3-(2-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-2-oxoethyl)-5,11-dimethyl-6,9-dioxo-2-oxa-5,8,11-triazapentadecan-15-oate

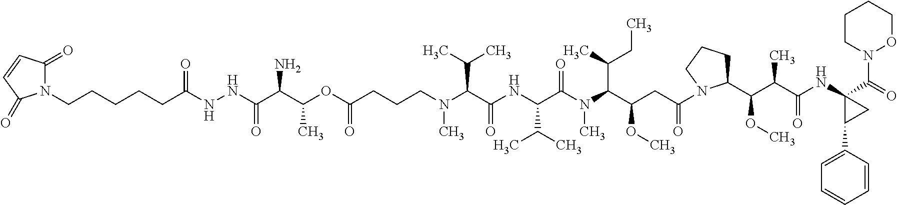 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)O[C@H](C)[C@H](N)C(=O)NNC(=O)CCCCCN1C(=O)C=CC1=O)C(C)C]

15 mg (17 μmol) of N-(3-carboxypropyl)-N-methyl-L-valyl-N-[(3R,4S,5S)-3-methoxy-1-{(2S)-2-[(1R,2R)-1-methoxy-2-methyl-3-{[(1S)-1-(1,2-oxazinan-2-yl)-2-phenylcyclopropyl]amino}-3-oxopropyl]pyrrolidin-1-yl}-5-methyl-1-oxoheptan-4-yl]-N-methyl-L-valinamide were dissolved in 10 ml of dichloromethane and then admixed with 12.8 mg (67 μmol) of 1-(3-dimethylaminopropyl)-3-ethylcarbodiimide hydrochloride, 10 mg (83 μmol) of 4-dimethylaminopyridine and with 10.3 mg (33 μmol) of commercially available benzyl N-(tert-butoxycarbonyl)-L-threoninate. The mixture was heated to reflux for 4 h. Then the same amounts of coupling reagent and 4-dimethylaminopyridine were added again and the reaction mixture was heated under reflux overnight. Subsequently, the reaction mixture was diluted with dichloromethane and extracted by shaking once with water, and the organic phase was removed and concentrated under high vacuum. The remaining residue was purified by means of preparative HPLC. Thus, 7.7 mg (37% of theory) of the protected intermediate were obtained as a colourless foam.

HPLC (Method 12): Rₜ=2 5 min;

LC-MS (Method 1): Rₜ=1.13 min; MS (ESIpos): m/z=1190 (M+H)⁺.

Subsequently, the benzyl ester protecting group was removed by hydrogenation under standard hydrogen pressure in methanol over 10% palladium/activated carbon, and the acid thus obtained, as described in Intermediate 151, was joined to 6-(2,5-dioxo-2,5-dihydro-1H-pyrrol-1-yl)hexanehydrazide. In a last step, the Boc protecting group was detached with trifluoroacetic acid. The remaining residue was purified by means of preparative HPLC. Thus, 0.22 mg (2.5% of theory over 3 stages) of the title compound was obtained as a colourless foam.

HPLC (Method 12): Rₜ=2.0 min;

LC-MS (Method 1): Rₜ=0.81 min; MS (ESIpos): m/z=1207 (M+H)⁺.

### Intermediate 155

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-amino-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

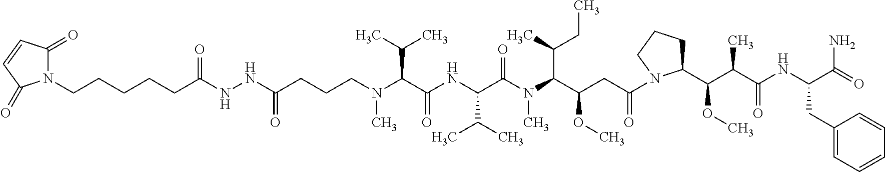 [SMILES: CC[C@H](C)[C@@H]([C@@H](CC(=O)N1CCC[C@H]1[C@H](OC)[C@@H](C)C(=O)N[C@@H](Cc1ccccc1)C(N)=O)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 152, from N-(3-carboxypropyl)-N-methyl-L-valyl-N-[(3R,4S,5S)-1-{(2S)-2-[(1R,2R)-3-{[(2S)-1-amino-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide and commercially available 6-(2,5-dioxo-2,5-dihydro-1H-pyrrol-1-yl)hexanehydrazide.

HPLC (Method 5): Rₜ=1.6 min;

LC-MS (Method 1): Rₜ=0.82 min; MS (ESIpos): m/z=1024 (M+H)⁺.

### Intermediate 156

### N-(3-{[(1-{[(2,5-dioxopyrrolidin-1-yl)oxy]carbonyl}cyclopropyl)carbonyl]amino}propyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

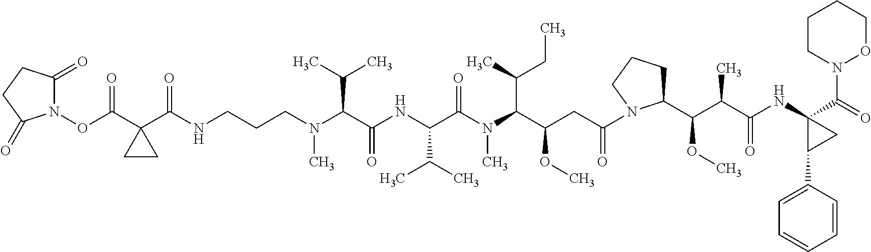 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCNC(=O)C1(C(=O)ON2C(=O)CCC2=O)CC1)C(C)C]

This compound was prepared in analogy to the synthesis described in the last stage of Intermediate 131, from N-(3-aminopropyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide and 1,1′-[cyclopropane-1,1-diylbis(carbonyloxy)]dipyrrolidine-2,5-dione, which had been obtained from the corresponding dicarboxylic acid beforehand.

HPLC (Method 12): Rₜ=2.0 min;

LC-MS (Method 1): Rₜ=0.92 min; MS (ESIpos): m/z=1080 (M+H)⁺.

### Intermediate 157

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

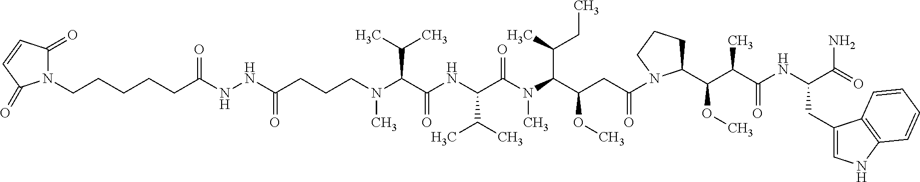 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)NNC(=O)CCCCCN1C(=O)C=CC1=O)C(C)C]

15 mg (18 μmol) of (N-(3-carboxypropyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 3.8 ml of DMF and then admixed with 27 mg (70 μmol) of O-(7-azabenzotriazol-1-yl)-N,N,N′,N′-tetramethyluronium hexafluorophosphate, 12 μl of N,N-diisopropylethylamine and with 14 mg (53 μmol) of commercially available 6-(2,5-dioxo-2,5-dihydro-1H-pyrrol-1-yl)hexanehydrazide. The reaction mixture was stirred at RT for 1 h. This was followed by concentration under high vacuum and purification of the remaining residue by means of preparative HPLC. Thus, 6.2 mg (33% of theory) of the title compound were obtained as a colourless foam.

HPLC (Method 5): Rₜ=1.6 min;

LC-MS (Method 1): Rₜ=0.85 min; MS (ESIpos): m/z=1063 (M+H)⁺.

¹H-NMR (500 MHz, DMSO-d₆, characteristic signals): δ=10.8 (d, 1H), 9.8-9.7 (m, 2H), 9.6 and 9.4 (2m, 1H), 8.9, 8.88, 8.78 and 8.75 (4d, 1H), 8.08 and 7.85 (2d, 1H), 7.6-6.9 (m, 9H), 4.7-4.4 (m, 3H), 3.4 (t, 2H), 3.23, 3.2, 3.18, 3.0, and 2.99 (5s, 9H), 2.8 (m, 3H), 2.1 (t, 2H), 1.06 and 1.01 (2d, 3H), 0.95-0.8 (m, 15H), 0.8-0.75 (dd, 3H).

### Intermediate 158

### N-[4-({(2R)-1-[(2,5-dioxopyrrolidin-1-yl)oxy]-4-methyl-1-oxopentan-2-yl}amino)-4-oxobutyl]-N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

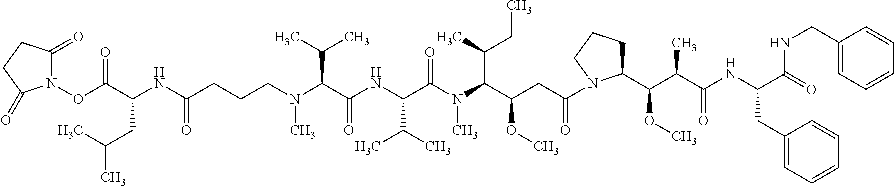 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCc1ccccc1)OC)N(C)C(=O)[C@@H](NC(=O)[C@H](C(C)C)N(C)CCCC(=O)N[C@H](CC(C)C)C(=O)ON1C(=O)CCC1=O)C(C)C]

13 mg (14.7 μmol) of N-(3-carboxypropyl)-N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 4 ml of dimethylformamide and then admixed with 9.4 mg (25 μmol) of O-(7-azabenzotriazol-1-yl)-N,N,N′,N′-tetramethyluronium hexafluorophosphate, 6 μl of N,N-diisopropylethylamine and with 7 mg (31 μmol) of commercially available tert-butyl D-leucinate hydrochloride trifluoroacetate. The mixture was stirred at RT for 5 h and then concentrated under reduced pressure. The remaining residue was purified by means of preparative HPLC. After lyophilization from dioxane/water, 6.5 mg (49% of theory) of the protected intermediate were obtained as a colourless foam.

HPLC (Method 5): Rₜ=2.2 min;

LC-MS (Method 1): Rₜ=1.21 min; MS (ESIpos): m/z=1076 (M+H)⁺.

Trifluoroacetic acid in dichloromethane was first used to detach the Boc protecting group from this protected intermediate, giving 6.2 mg (99% of theory) of the deprotected compound. 5.2 mg (5 μmol) of this intermediate were taken up in 1.5 ml of dichloromethane and reacted with 0.8 mg (7 μmol) of N-hydroxysuccinimide in the presence of 1.2 mg (6 μmol) of 1-(3-dimethylaminopropyl)-3-ethylcarbodiimide hydrochloride and 0.16 mg (1 μmol) of 4-dimethylaminopyridine. After stirring at RT for 2 h, the reaction mixture was concentrated and purified by means of preparative HPLC. 1.3 mg of the title compound were obtained, some of which was hydrolysed to the reactant.

### Intermediate 159

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

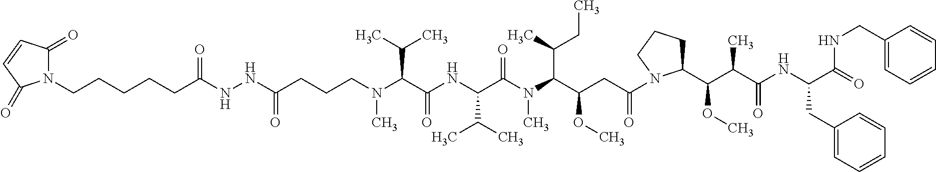 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCc1ccccc1)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 157, from N-(3-carboxypropyl)-N-methyl-L-valyl-N-[(3R,4S,5S)-1-{(2S)-2-[(1R,2R)-3-{[(2S)-1-(benzylamino)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide and commercially available 6-(2,5-dioxo-2,5-dihydro-1H-pyrrol-1-yl)hexanehydrazide.

Yield: 6 mg (53% of theory)

HPLC (Method 5): Rₜ=1.9 min;

LC-MS (Method 1): Rₜ=0.94 min; MS (ESIpos): m/z=1114 (M+H)⁺.

### Intermediate 160

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

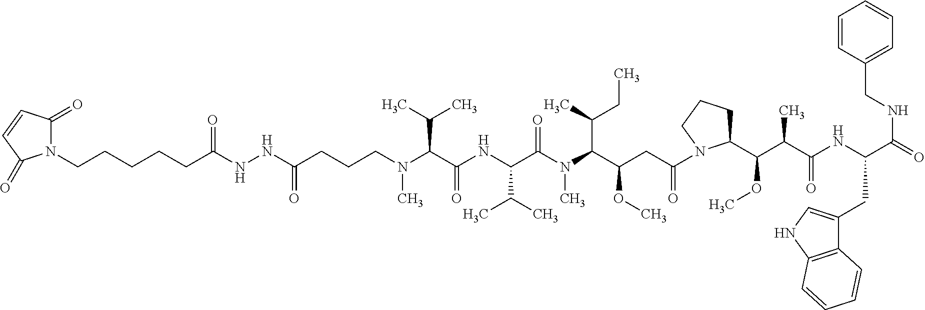 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)NCc1ccccc1)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 157, from 20 mg (21 μmol) of N-(3-carboxypropyl)-N-methyl-L-valyl-N-[(3R,4S,5S)-1-{(2S)-2-[(1R,2R)-3-{[(2S)-1-(benzylamino)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide and commercially available 6-(2,5-dioxo-2,5-dihydro-1H-pyrrol-1-yl)hexanehydrazide.

Yield: 13 mg (52% of theory)

HPLC (Method 5): Rₜ=1.9 min;

LC-MS (Method 1): Rₜ=0.92 min; MS (ESIpos): m/z=1153 (M+H)⁺.

### Intermediate 161

### N-(6-{2-[6-(2,5-dioxo-2,5-dihydro-1H-pyrrol-1-yl)hexanoyl]hydrazino}-6-oxohexyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

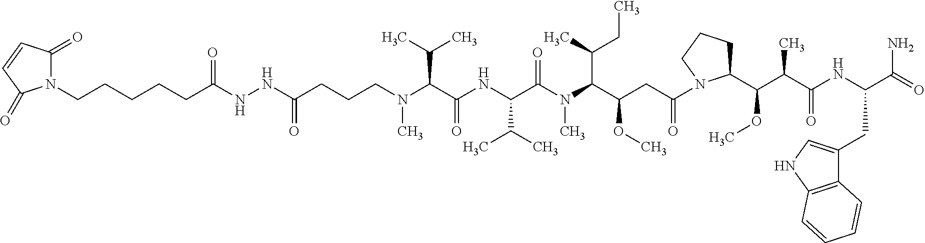 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 157, from N-(5-carboxypentyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide and commercially available 6-(2,5-dioxo-2,5-dihydro-1H-pyrrol-1-yl)hexanehydrazide.

Yield: 0.8 mg (16% of theory)

HPLC (Method 5): Rₜ=1.6 min;

LC-MS (Method 1): Rₜ=0.78 min; MS (ESIpos): m/z=1092 (M+H)⁺.

### Intermediate 162

### N-{6-[(2,5-dioxopyrrolidin-1-yl)oxy]-6-oxohexyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

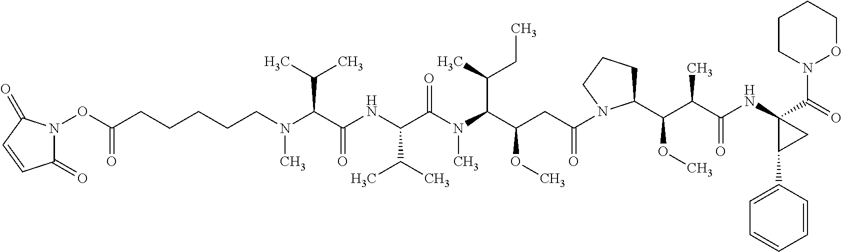 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCCCC(=O)ON1C(=O)C=CC1=O)C(C)C]

18 mg (20 μmol) of N-(5-carboxypentyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide (Intermediate 64) were dissolved in 3.2 ml of dichloromethane and admixed with 22 mg (190 mmol) of 1-hydroxypyrrolidine-2,5-dione and then with 11 mg (60 μmol) of 1-(3-dimethylaminopropyl)-3-ethylcarbodiimide hydrochloride and 0.24 mg (0.17 μmol) of DMAP. After stirring at RT for 2 h, another 22 mg (190 mmol) of 1-hydroxypyrrolidine-2,5-dione, 11 mg (60 μmol) of 1-(3-dimethylaminopropyl)-3-ethylcarbodiimide hydrochloride and 0.24 mg (0.17 μmol) of DMAP were added and the reaction mixture was stirred at RT for a further hour. This was followed by concentration under reduced pressure. The remaining residue was purified by means of preparative HPLC. After lyophilization, 8.2 mg (41% of theory) of the title compound were obtained.

HPLC (Method 5): Rₜ=2.0 min;

LC-MS (Method 11): Rₜ=0.9 min; MS (ESIpos): m/z=1024 (M+H)⁺.

### Intermediate 163

### [(1S,2R)-1-amino-2-phenylcyclopropyl](1,4-dihydro-3H-2,3-benzoxazin-3-yl)methanone trifluoroacetate

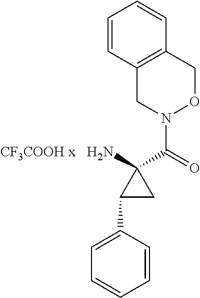 [SMILES: N[C@@]1(C(=O)N2Cc3ccccc3CO2)C[C@@H]1c1ccccc1]

First, proceeding from 265 mg (0.82 mmol) of tert-butyl (1S,2R)-1-(hydroxycarbamoyl)-2-phenylcyclopropyl carbamate (Starting Compound 7), by reaction with 1,2-bis(bromomethyl)benzene, analogously to a literature method (see H. King, J. Chem. Soc. 1942, 432), the Boc-protected tert-butyl (1S,2R)-1-(1,4-dihydro-3H-2,3-benzoxazin-3-ylcarbonyl)-2-phenylcyclopropyl carbamate intermediate was prepared.

Yield: 108 mg (34% of theory)

LC-MS (Method 2): Rₜ=1.3 min; MS (ESIpos): m/z=395 (M+H)⁺.

108 mg (0.27 mmol) of this intermediate were taken up in 3.7 ml of dichloromethane, 1.8 ml of trifluoroacetic acid were added, and the mixture was stirred at RT for 15 min. This was followed by concentration under reduced pressure and lyophilization of the remaining residue from dioxane. 112 mg of the title compound were obtained in quantitative yield as a colourless foam.

LC-MS (Method 1): Rₜ=0.7 min; MS (ESIpos): m/z=295 (M+H)⁺.

### Intermediate 164

### N-methyl-L-valyl-N-[(3R,4S,5S)-1-{(2S)-2-[(1R,2R)-3-{[(1S,2R)-1-(1,4-dihydro-3H-2,3-benzoxazin-3-ylcarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

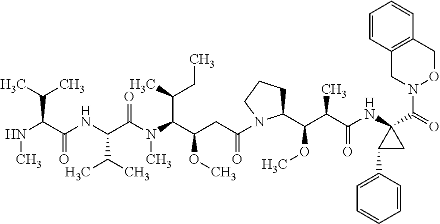 [SMILES: CC[C@H](C)[C@@H]([C@@H](CC(=O)N1CCC[C@H]1[C@H](OC)[C@@H](C)C(=O)N[C@@]1(C(=O)N2Cc3ccccc3CO2)C[C@@H]1c1ccccc1)OC)N(C)C(=O)[C@@H](NC(=O)[C@@H](NC)C(C)C)C(C)C]

166 mg (0.196 mmol) of N-[(9H-fluoren-9-ylmethoxy)carbonyl]-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 10) were taken up in 40 ml of DMF and admixed successively with 80 mg (0.196 mmol) of [(1S,2R)-1-amino-2-phenylcyclopropyl](1,4-dihydro-3H-2,3-benzoxazin-3-yl)methanone trifluoroacetate (Intermediate 163), 112 mg (0.294 mmol) of O-(7-azabenzotriazol-1-yl)-N,N,N′,N′-tetramethyluronium hexafluorophosphate (HATU) and 682 μl (3.9 mmol) of N,N-diisopropylethylamine. The mixture was subsequently stirred at RT overnight. The reaction mixture was then concentrated under reduced pressure, the residue was taken up in ethyl acetate and the solution was washed with saturated aqueous sodium chloride solution. The organic phase was dried over magnesium sulphate, filtered and concentrated. The residue was finally purified by preparative HPLC. In this way, 19 mg (9% of theory) of the Fmoc-protected intermediate N-[(9H-fluoren-9-ylmethoxy)carbonyl]-N-methyl-L-valyl-N-[(3R,4S,5S)-1-{(2S)-2-[(1R,2R)-3-{[(1S,2R)-1-(1,4-dihydro-3H-2,3-benzoxazin-3-ylcarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide were obtained.

HPLC (Method 5): Rₜ=1.68 min;

LC-MS (Method 1): Rₜ=1.51 min; MS (ESIpos): m/z=1083 (M+H)⁺.

19 mg (0.015 mmol) of this intermediate were dissolved in 4 ml of DMF. After 817 μl of piperidine had been added, the reaction mixture was stirred at RT for 5 min. This was followed by concentration under reduced pressure, and the residue was first digested with diethyl ether and then purified by means of preparative HPLC (eluent: acetonitrile+0.1% TFA/0.1% aq. TFA). The corresponding fractions were combined, the solvent was removed under reduced pressure and then the residue was lyophilized from dioxane/water. 12 mg (92% of theory) of the title compound were obtained as a colourless foam.

HPLC (Method 6): Rₜ=2.0 min;

LC-MS (Method 1): Rₜ=0.94 min; MS (ESIpos): m/z=861 (M+H)⁺.

### Intermediate 165

### N-(6-aminohexyl)-N-methyl-L-valyl-N-[(3R,4S,5S)-1-{(2S)-2-[(1R,2R)-3-{[(1S,2R)-1-(1,4-dihydro-3H-2,3-benzoxazin-3-ylcarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

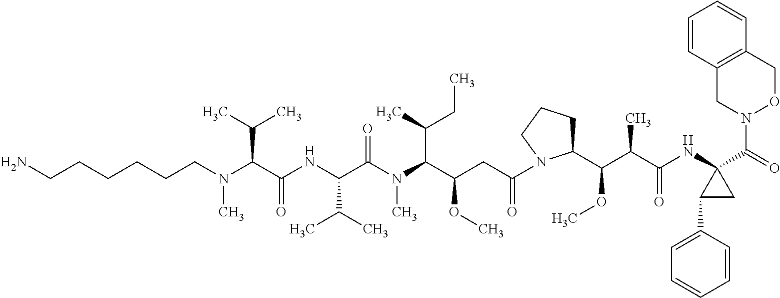 [SMILES: CC[C@H](C)[C@@H]([C@@H](CC(=O)N1CCC[C@H]1[C@H](OC)[C@@H](C)C(=O)N[C@@]1(C(=O)N2Cc3ccccc3CO2)C[C@@H]1c1ccccc1)OC)N(C)C(=O)[C@@H](NC(=O)[C@H](C(C)C)N(C)CCCCCCN)C(C)C]

20 mg (0.021 mmol) of Intermediate 164 were used, in analogy to the preparation of Intermediate 97, with benzyl 3-oxohexyl carbamate in the presence of sodium cyanoborohydride and subsequent hydrogenolytic detachment of the Z protecting group (with 5% palladium on charcoal as a catalyst, in methanol as a solvent), to prepare the title compound.

Yield: 4.5 mg (23% of theory over 2 stages)

HPLC (Method 12): Rₜ=1.9 min;

LC-MS (Method 1): Rₜ=0.9 min; MS (ESIpos): m/z=960 (M+H)⁺.

### Intermediate 166

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(1S,2R)-1-(1,4-dihydro-3H-2,3-benzoxazin-3-ylcarbonyl)-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

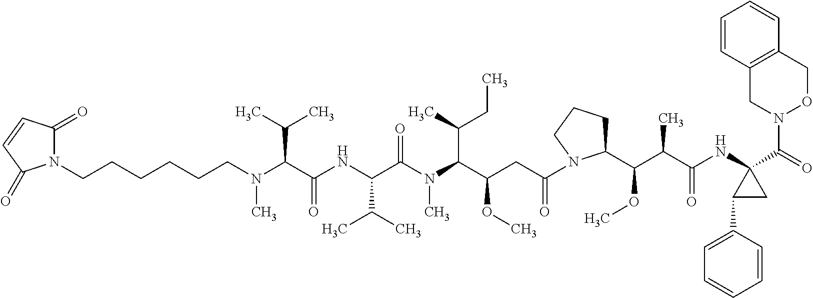 [SMILES: CC[C@H](C)[C@@H]([C@@H](CC(=O)N1CCC[C@H]1[C@H](OC)[C@@H](C)C(=O)N[C@@]1(C(=O)N2Cc3ccccc3CO2)C[C@@H]1c1ccccc1)OC)N(C)C(=O)[C@@H](NC(=O)[C@H](C(C)C)N(C)CCCCCCN1C(=O)C=CC1=O)C(C)C]

4.4 mg (4.5 μmol) of Intermediate 165 were taken up in 1 ml of 1:1 dioxane/water and then admixed with 1 mg (6.8 μmol) of methyl 2,5-dioxo-2,5-dihydro-1H-pyrrole-1-carboxylate and with 50 μl of saturated aqueous sodium hydrogencarbonate solution. The reaction mixture was stirred at RT for 30 min. Then another 50 μl of the saturated aqueous sodium hydrogencarbonate solution were added and the reaction mixture was stirred at RT for a further 15 min and then concentrated under reduced pressure. The remaining residue was purified by means of preparative HPLC. After lyophilization, 1 mg (21% of theory) of the title compound were obtained as a colourless foam.

HPLC (Method 12): Rₜ=2.1 min;

LC-MS (Method 1): Rₜ=1.08 min; MS (ESIpos): m/z=1040 (M+H)⁺.

### Intermediate 167

### benzyl 3-{2-[2-(2-oxoethoxy)ethoxy]ethoxy}propanoate

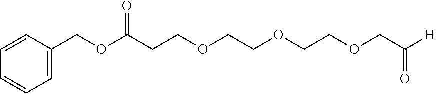 [SMILES: O=CCOCCOCCOCCC(=O)OCc1ccccc1]

The title compound was prepared from 6 g (21.55 mmol) of commercially available 3-{2-[2-(2-hydroxyethoxy)ethoxy]ethoxy}propanoic acid under standard conditions, first by esterification with benzyl chloride and caesium carbonate and subsequent oxidation with sulphur trioxide-pyridine complex.

Yield: 611 mg (10% of theory over 2 stages)

LC-MS (Method 2): Rₜ=1.69 min; MS (ESIpos): m/z=311 (M+H)⁺.

### Intermediate 168

### N-(2-{2-[2-(2-carboxyethoxy)ethoxy]ethoxy}ethyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

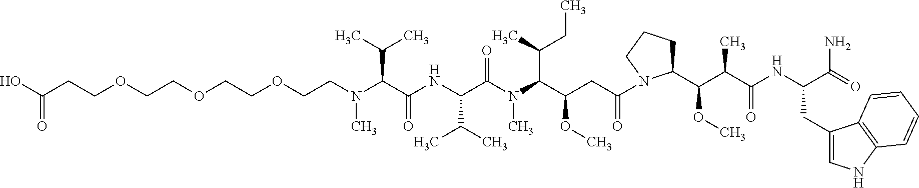 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCOCCOCCOCCC(=O)O)C(C)C]

First, in analogy to the synthesis described in Intermediate 69, by coupling of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) and N^{α}-{(2R,3R)-3-methoxy-2-methyl-3-[(2S)-pyrrolidin-2-yl]propanoyl}-L-tryptophanamide trifluoroacetate (Intermediate 49) in the presence of O-(7-azabenzotriazol-1-yl)-N,N,N′,N′-tetramethyluronium hexafluorophosphate and subsequent detachment of the Fmoc protecting group by means of piperidine, the amine compound N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate.

25 mg (0.028 mmol) of this compound and 17.5 mg (0.06 mmol) of Intermediate 167 were combined in 2 ml of methanol and admixed with 12.6 mg (0.14 mmol) of borane-pyridine complex and 2.5 ml of acetic acid. The reaction mixture was stirred at RT overnight. Then the same amounts of borane-pyridine complex and acetic acid were added once again and the reaction mixture was stirred at RT for a further 24 h. This was followed by concentration under reduced pressure, and the residue was purified by means of preparative HPLC. After concentration of the corresponding fractions and lyophilization from 1:1 dioxane/water, 26.5 mg (88% of theory) of the Z-protected title compound were obtained.

HPLC (Method 12): Rₜ=2.04 min;

LC-MS (Method 1): Rₜ=0.97 min; MS (ESIpos): m/z=1064 (M+H)⁺.

25 mg (0.024 mmol) of this intermediate were taken up in 10 ml of methanol and hydrogenated over 10% palladium on activated carbon under standard hydrogen pressure at RT for 45 min. The catalyst was then filtered off and the solvent was removed under reduced pressure. After lyophilization from dioxane, 19.7 mg (85% of theory) of the title compound were obtained.

HPLC (Method 12): Rₜ=1.8 min;

LC-MS (Method 1): Rₜ=0.83 min; MS (ESIpos): m/z=974 (M+H)⁺.

### Intermediate 169

### N-{2-[2-(2-{3-[(2,5-dioxopyrrolidin-1-yl)oxy]-3-oxopropoxy}ethoxy)ethoxy]ethyl}-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

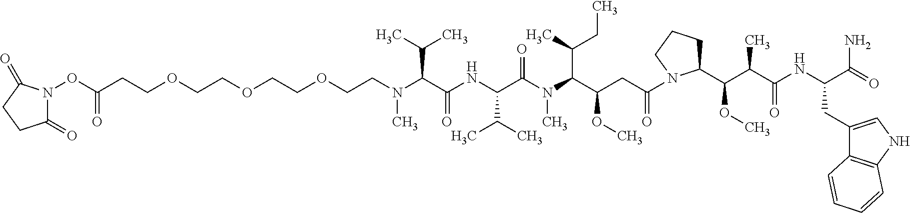 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCOCCOCCOCCC(=O)ON1C(=O)CCC1=O)C(C)C]

10 mg (10 μmol) of Intermediate 168 were dissolved in 3 ml of DMF and admixed with 3.5 mg (30 mmol) of 1-hydroxypyrrolidine-2,5-dione and then with 2.4 mg (10 μmol) of 1-(3-dimethylaminopropyl)-3-ethylcarbodiimide hydrochloride and 5 μl of N,N-diisopropylethylamine. After stirring at RT for 20 h, 8 mg (0.02 mmol) of HATU were added and the reaction mixture was stirred once again at RT overnight and then concentrated under reduced pressure. The remaining residue was purified by means of preparative HPLC. After lyophilization from dioxane, 8.6 mg (64% of theory) of the title compound were obtained.

HPLC (Method 12): Rt=1.9 min;

LC-MS (Method 11): Rt=0.81 min; MS (ESIpos): m/z=1071 (M+H)+.

### Intermediate 170

### N-(6-aminohexyl)-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide

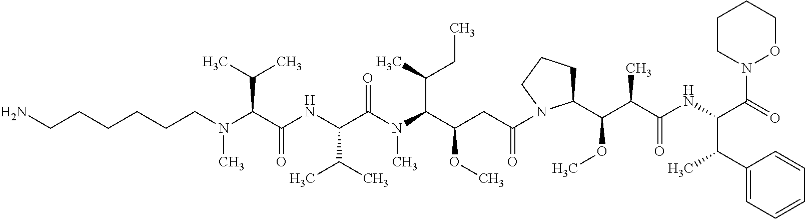 [SMILES: CC[C@H](C)[C@@H]([C@@H](CC(=O)N1CCC[C@H]1[C@H](OC)[C@@H](C)C(=O)N[C@H](C(=O)N1CCCCO1)[C@@H](C)c1ccccc1)OC)N(C)C(=O)[C@@H](NC(=O)[C@H](C(C)C)N(C)CCCCCCN)C(C)C]

This compound was prepared in analogy to Intermediate 101 over 2 stages, proceeding from 26 mg (0.028 mmol) of Intermediate 15.

Yield: 16.7 mg (63% of theory over 2 stages)

HPLC (Method 12): Rₜ=1.9 min;

LC-MS (Method 1): Rₜ=0.81 min; MS (ESIpos): m/z=914 (M+H)⁺.

### Intermediate 171

### N-(6-{[4-(2,5-dioxo-2,5-dihydro-1H-pyrrol-1-yl)butanoyl]amino}hexyl)-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide

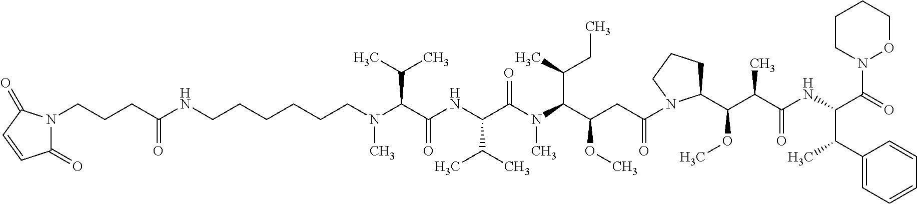 [SMILES: CC[C@H](C)[C@@H]([C@@H](CC(=O)N1CCC[C@H]1[C@H](OC)[C@@H](C)C(=O)N[C@H](C(=O)N1CCCCO1)[C@@H](C)c1ccccc1)OC)N(C)C(=O)[C@@H](NC(=O)[C@H](C(C)C)N(C)CCCCCCNC(=O)CCCN1C(=O)C=CC1=O)C(C)C]

6.7 mg (7.3 μmol) of the compound formed from Intermediate 170 and 3 mg (14.7 μmol) of commercially available 4-(2,5-dioxo-2,5-dihydro-1H-pyrrol-1-yl)butanoic acid were taken up in 2 ml of DMF and admixed with 5.6 mg (14.7 μmol) of O-(7-azabenzotriazol-1-yl)-N,N,N′,N′-tetramethyluronium hexafluorophosphate (HATU) and 2 μl of N,N-diisopropylethylamine. The mixture was stirred at RT for 30 min. The reaction mixture was concentrated and the residue was purified by means of preparative HPLC. The corresponding fractions were combined, the solvent was removed under reduced pressure and then the residue was lyophilized from dioxane. Thus, 4.5 mg (56% of theory) of the title compound were obtained.

HPLC (Method 12): Rₜ=2.0 min;

LC-MS (Method 1): Rₜ=1.12 min; MS (ESIpos): m/z=1079 (M+H)⁺.

### Intermediate 172

### benzyl 2-{2-[2-(2-oxoethoxy)ethoxy]ethoxy}ethyl carbamate

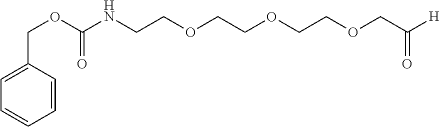 [SMILES: O=CCOCCOCCOCCNC(=O)OCc1ccccc1]

The title compound was prepared from commercially available 2-{2-[2-(2-aminoethoxy)ethoxy]ethoxy}ethanol under standard conditions, by first introducing the Z protecting group and then oxidizing with sulphur trioxide-pyridine complex.

HPLC (Method 12): Rₜ=1.4 min;

LC-MS (Method 11): Rₜ=0.65 min; MS (ESIpos): m/z=326 (M+H)⁺.

### Intermediate 173

### benzyl {2-[2-(2-oxoethoxy)ethoxy]ethyl carbamate

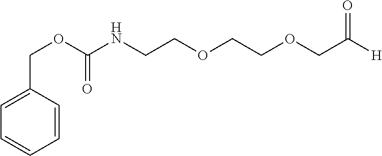 [SMILES: O=CCOCCOCCNC(=O)OCc1ccccc1]

The title compound was prepared analogously to Intermediate 172 from commercially available 2-[2-(2-aminoethoxy)ethoxy]ethanol under standard conditions, by first introducing the Z protecting group and then oxidizing with sulphur trioxide-pyridine complex.

HPLC (Method 12): Rₜ=1.3 min;

LC-MS (Method 11): Rₜ=0.68 min; MS (ESIpos): m/z=282 (M+H)⁺.

### Intermediate 174

### N-(2-{2-[2-(2-aminoethoxy)ethoxy]ethoxy}ethyl)-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenyl cyclopropyl]amino}-3-oxopropyl]pyrrolidin-1-yl}-5-methyl-1-oxoheptan-4-yl]-N-methyl-L-valinamide

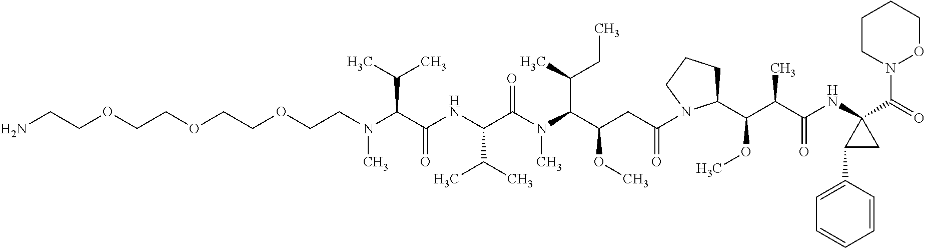 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCOCCOCCOCCN)C(C)C]

47 mg (0.05 mmol) of Intermediate 16 were reductively aminated in analogy to the preparation of Intermediate 167 with benzyl 2-{2-[2-(2-oxoethoxy)ethoxy]ethoxy}ethyl carbamate in the presence of borane-pyridine complex. Subsequently, the Z protecting group was removed by hydrogenolytic means with 5% palladium on charcoal as a catalyst and in methanol as a solvent, and 38 mg (66% of theory over 2 stages) of the title compound were prepared.

HPLC (Method 5): Rₜ=1.7 min;

LC-MS (Method 1): Rₜ=0.8 min; MS (ESIpos): m/z=988 (M+H)⁺.

### Intermediate 175

### N-[2-(2-{2-[2-(2,5-dioxo-2,5-dihydro-1H-pyrrol-1-yl)ethoxy]ethoxy}ethoxy)ethyl]-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

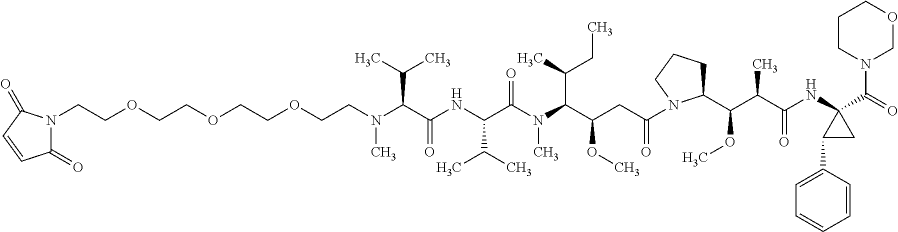 [SMILES: CC[C@H](C)[C@@H]([C@@H](CC(=O)N1CCC[C@H]1[C@H](OC)[C@@H](C)C(=O)N[C@@]1(C(=O)N2CCCOC2)C[C@@H]1c1ccccc1)OC)N(C)C(=O)[C@@H](NC(=O)[C@H](C(C)C)N(C)CCOCCOCCOCCN1C(=O)C=CC1=O)C(C)C]

The preparation was effected in analogy zu Intermediate 166, proceeding from 34 mg (0.03 mmol) of Intermediate 174.

Yield: 8.3 mg (23% of theory)

HPLC (Method 5): Rₜ=1.9 min;

LC-MS (Method 1): Rₜ=0.97 min; MS (ESIpos): m/z=1068 (M+H)⁺.

### Intermediate 176

### N-(2-{2-[2-(2,5-dioxo-2,5-dihydro-1H-pyrrol-1-yl)ethoxy]ethoxy}ethyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

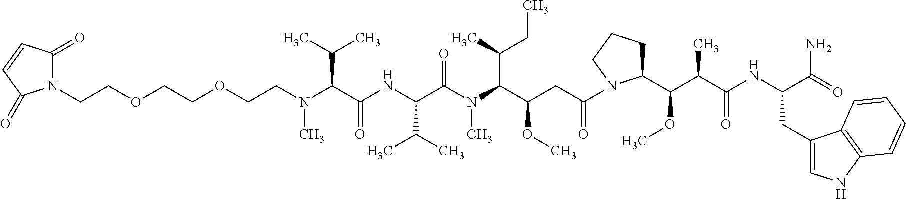 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCOCCOCCN1C(=O)C=CC1=O)C(C)C]

The preparation was effected in analogy to Intermediates 174 and 175, commencing with the reductive amination of Intermediate 16 with Intermediate 173, subsequent deprotection and formation of the maleimide.

HPLC (Method 12): Rₜ=1.8 min;

LC-MS (Method 11): Rₜ=0.8 min; MS (ESIpos): m/z=981 (M+H)⁺.

### Intermediate 177

### N-[2-(2-{2-[2-(2,5-dioxo-2,5-dihydro-1H-pyrrol-1-yl)ethoxy]ethoxy}ethoxy)ethyl]-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

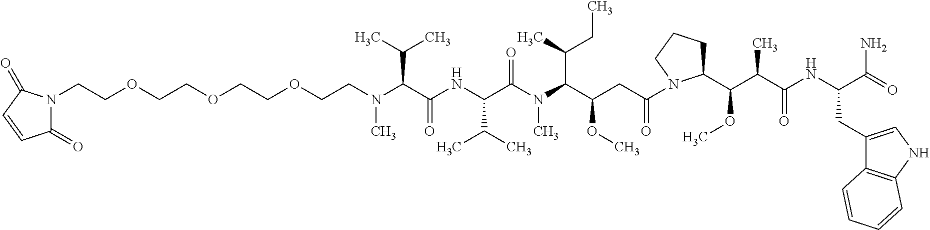 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCOCCOCCOCCN1C(=O)C=CC1=O)C(C)C]

The preparation was effected in analogy to Intermediates 174 and 175, commencing with the reductive amination of Intermediate 16 with Intermediate 172, subsequent deprotection and formation of the maleimide.

HPLC (Method 12): Rₜ=1.9 min;

LC-MS (Method 1): Rₜ=0.86 min; MS (ESIpos): m/z=1025 (M+H)⁺.

### Intermediate 178

### N-{4-[(2,5-dioxopyrrolidin-1-yl)oxy]-4-oxobutyl}-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

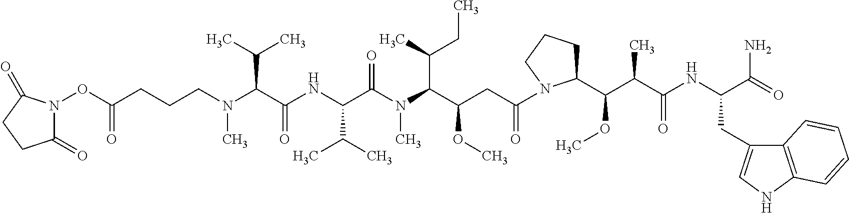 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)ON1C(=O)CCC1=O)C(C)C]

The preparation was effected in analogy to Intermediates 162, proceeding from 6 mg of Intermediate 82.

LC-MS (Method 1): Rₜ=0.82 min; MS (ESIpos): m/z=953 (M+H)⁺.

### Intermediate 179

### 4-[(1E,3S)-3-amino-4-phenylbut-1-en-1-yl]benzenesulphonic acid trifluoroacetate

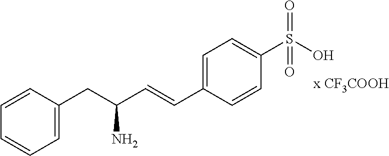 [SMILES: N[C@H](/C=C/c1ccc(S(=O)(=O)O)cc1)Cc1ccccc1]

A mixture of 13.6 mg (0.06 mmol) of palladium(II) acetate, 469 mg (1.46 mmol) of potassium 4-iodobenzenesulphonate, 300 mg (1.21 mmol) of (S)-tert-butyl 1-phenylbut-3-en-2-yl carbamate, 16.5 mg (0.12 mmol) of phenylurea and 167.6 mg (1.21 mmol) of potassium carbonate in 7.5 ml of DMF was heated to 160° C. in a microwave for 15 min. The crude product was subsequently purified directly by preparative HPLC. This gave 312 mg of a mixture of 31% of the BOC-protected compound and 69% of the free amine

This mixture was subsequently taken up in 30 ml of dichloromethane, admixed with 1 ml of trifluoroacetic acid and stirred at RT for 20 h. After concentrating under reduced pressure, the residue was stirred with diethyl ether, and the precipitate formed was filtered off with suction and washed with diethyl ether. This gave 200 mg (62% of theory) of the title compound.

LC-MS (Method 11): Rₜ=0.44 min; MS (ESIpos): m/z=304 (M+H)⁺.

### Intermediate 180

### 4-[(3R)-3-amino-4-phenylbutyl]benzenesulphonic acid

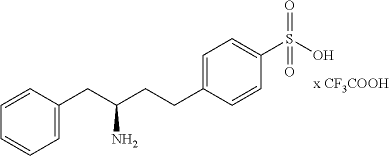 [SMILES: N[C@H](CCc1ccc(S(=O)(=O)O)cc1)Cc1ccccc1]

100 mg (0.25 mmol) of 4-[(1E,3S)-3-amino-4-phenylbut-1-en-1-yl]benzenesulphonic acid trifluoroacetate were suspended in 10 ml of acetic acid and a few drops of DMF and water, admixed with 70 mg (0.07 mmol) of palladium on charcoal (10%) and hydrogenated at hydrogen pressure 2.2 bar for 24 h. The solution was filtered and the filtrate purified by prep. HPLC.

29 mg (76% purity, 21% of theory) of product were obtained.

LC-MS (Method 1): Rₜ=0.46 min; MS (ESIpos): m/z=306 (M+H)⁺.

### Intermediate 181

### N-(tert-butoxycarbonyl)-N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide

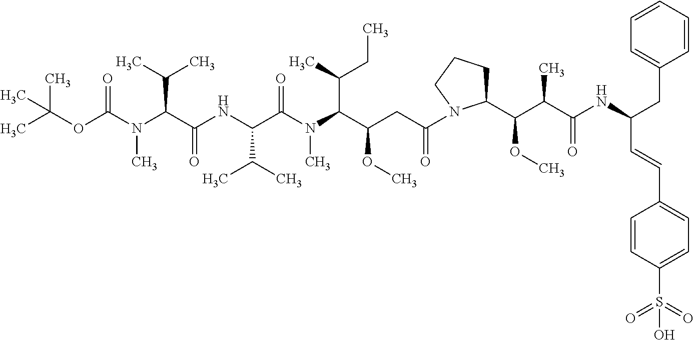 [SMILES: CC[C@H](C)[C@@H]([C@@H](CC(=O)N1CCC[C@H]1[C@H](OC)[C@@H](C)C(=O)N[C@H](/C=C/c1ccc(S(=O)(=O)O)cc1)Cc1ccccc1)OC)N(C)C(=O)[C@@H](NC(=O)[C@H](C(C)C)N(C)C(=O)OC(C)(C)C)C(C)C]

To a solution of 90 mg (0.13 mmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide in 4 ml of DMF were added 60 mg (0.16 mmol) of HATU and 69 μl of (0.39 mmol) Hünig's base. The reaction mixture was stirred at RT for 30 min and then admixed with 60 mg (0.15 mmol) 60.3 mg (0.13 mmol) of 4-[(1E,3S)-3-amino-4-phenylbut-1-en-1-yl]benzenesulphonic acid trifluoroacetate. After stirring overnight, the reaction mixture was purified by prep. HPLC. This gave 127 mg of a 44:56 mixture of the title compound and of the already deprotected amine.

LC-MS (Method 1): Rₜ=1.21 min; MS (ESIpos): m/z=971 (M+H)⁺; Rₜ=0.84 min; MS (ESIpos): m/z=871 (M+H)⁺ for the deprotected compound.

### Intermediate 182

### N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide trifluoroacetate

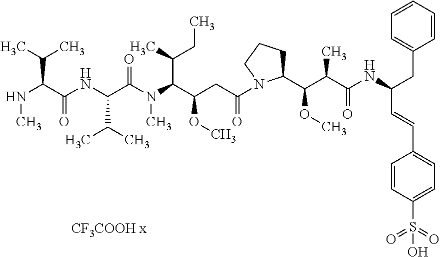 [SMILES: CC[C@H](C)[C@@H]([C@@H](CC(=O)N1CCC[C@H]1[C@H](OC)[C@@H](C)C(=O)N[C@H](/C=C/c1ccc(S(=O)(=O)O)cc1)Cc1ccccc1)OC)N(C)C(=O)[C@@H](NC(=O)[C@@H](NC)C(C)C)C(C)C]

90 mg of Intermediate 180 were dissolved in 4.6 ml of dichloromethane, and 0.92 ml of trifluoroacetic acid was added. The reaction mixture was stirred at RT for 30 min and then concentrated. The crude product obtained was purified by prep. HPLC.

91 mg (98% of theory) of the target compound were obtained.

LC-MS (Method 1): Rₜ=0.85 min; MS (ESIpos): m/z=871 (M+H)⁺

### Intermediate 183

### N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide

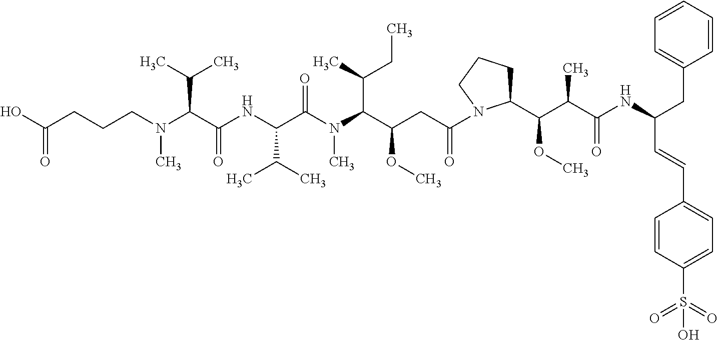 [SMILES: CC[C@H](C)[C@@H]([C@@H](CC(=O)N1CCC[C@H]1[C@H](OC)[C@@H](C)C(=O)N[C@H](/C=C/c1ccc(S(=O)(=O)O)cc1)Cc1ccccc1)OC)N(C)C(=O)[C@@H](NC(=O)[C@H](C(C)C)N(C)CCCC(=O)O)C(C)C]

16.7 μl (0.03 mmol) of a 15% aqueous succinaldehyde solution were initially charged in 943 μl of methanol and admixed with 17 mg (0.02 mmol) of N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide trifluoroacetate (Intermediate 181) and 1.1 μl (0.02 mmol) of acetic acid. The reaction mixture was stirred for 5 min at RT and then 2.9 μl (0.02 mmol) of borane-pyridine complex were added. After 1 h, a further 2 equivalents each of succinaldehyde, acetic acid and borane-pyridine complex were added and the mixture was stirred at RT for 20 h. The reaction mixture was then purified by prep. HPLC.

This gave 20 mg (83% purity, 80% of theory) of the title compound.

LC-MS (Method 1): Rₜ=0.87 min; MS (ESIpos): m/z=957 (M+H)⁺

### Intermediate 184

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide

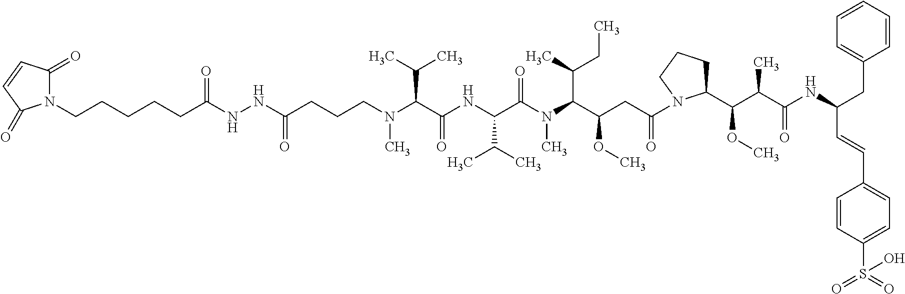 [SMILES: CC[C@H](C)[C@@H]([C@@H](CC(=O)N1CCC[C@H]1[C@H](OC)[C@@H](C)C(=O)N[C@H](/C=C/c1ccc(S(=O)(=O)O)cc1)Cc1ccccc1)OC)N(C)C(=O)[C@@H](NC(=O)[C@H](C(C)C)N(C)CCCC(=O)NNC(=O)CCCCCN1C(=O)C=CC1=O)C(C)C]

8 mg (7.5 μmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide, 2.8 mg (8.2 μmol) of 6-(2,5-dioxo-2,5-dihydro-1H-pyrrol-1-yl)hexanehydrazide trifluoroacetate, 3.4 mg (9 μmol) of HATU and 3.9 μl of Hünig's base were stirred in 0.77 ml of DMF at RT for 20 h. Subsequently, the reaction mixture was purified by prep. HPLC.

3 mg (31% of theory) of the title compound were obtained.

LC-MS (Method 1): Rₜ=0.90 min; MS (ESIpos): m/z=1164 (M+H)⁺

### Intermediate 185

### N-{4-[(2,5-dioxopyrrolidin-1-yl)oxy]-4-oxobutyl}-N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide

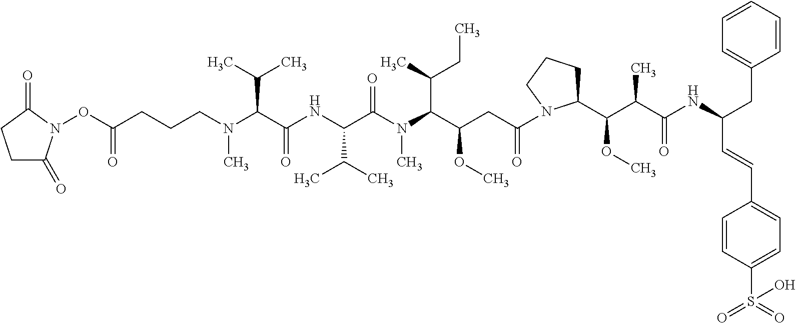 [SMILES: CC[C@H](C)[C@@H]([C@@H](CC(=O)N1CCC[C@H]1[C@H](OC)[C@@H](C)C(=O)N[C@H](/C=C/c1ccc(S(=O)(=O)O)cc1)Cc1ccccc1)OC)N(C)C(=O)[C@@H](NC(=O)[C@H](C(C)C)N(C)CCCC(=O)ON1C(=O)CCC1=O)C(C)C]

To a solution of 8 mg (7.5 μmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(2S,3E)-1-phenyl-4-(4-sulphophenyl)but-3-en-2-yl]amino}propyl]pyrrolidin-1-yl}-5-methyl-1-oxoheptan-4-yl]-N-methyl-L-valinamide in 2 ml of DMF were added 8.6 mg (74.8 μmol) of N-hydroxysuccinimide, 8.5 mg (22.4 μmol) of EDCI and 0.1 mg (0.75 μmol) of DMAP. The reaction mixture was stirred at RT for 20 h. Subsequently, 1.3 μl (7.5 μmol) of Hünig's base were added and the mixture was stirred for 1 h. The reaction mixture was then purified by prep. HPLC. 2.6 mg (72% purity, 21% of theory) of the title compound were obtained.

LC-MS (Method 1): Rₜ=0.89 min; MS (ESIpos): m/z=1054 (M+H)⁺

### Intermediate 186

### N-(tert-butoxycarbonyl)-N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide

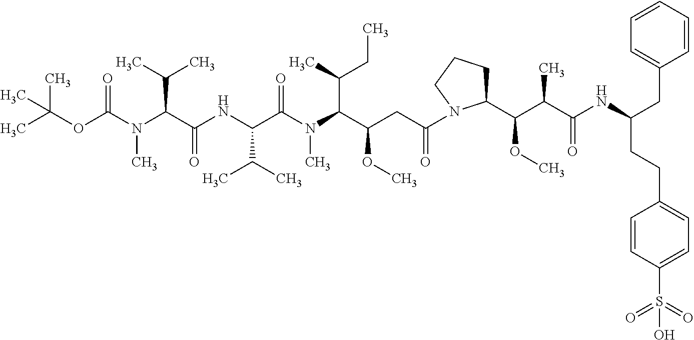 [SMILES: CC[C@H](C)[C@@H]([C@@H](CC(=O)N1CCC[C@H]1[C@H](OC)[C@@H](C)C(=O)N[C@H](CCc1ccc(S(=O)(=O)O)cc1)Cc1ccccc1)OC)N(C)C(=O)[C@@H](NC(=O)[C@H](C(C)C)N(C)C(=O)OC(C)(C)C)C(C)C]

To a solution of 43 mg (0.06 mmol) of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide in 1.9 ml of DMF were added 29 mg (0.07 mmol) of HATU and 33 μl (0.19 mmol) of Hünig's base. The reaction mixture was stirred at RT for 30 min and then admixed with 29 mg (0.07 mmol) of 4-[(3R)-3-amino-4-phenylbutyl]benzenesulphonic acid trifluoroacetate. After stirring overnight, the reaction mixture was purified by prep. HPLC. This gave 58 mg of a 45:55 mixture of the title compound and of the already deprotected amine.

LC-MS (Method 1): Rₜ=1.09 min; MS (ESIpos): m/z=973 (M+H)⁺; Rₜ=0.87 min; MS (ESIpos): m/z=873 (M+H)⁺ for the deprotected compound.

### Intermediate 187

### N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide trifluoroacetate

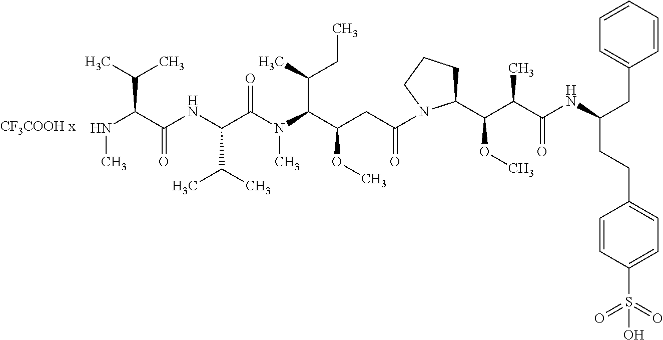 [SMILES: CC[C@H](C)[C@@H]([C@@H](CC(=O)N1CCC[C@H]1[C@H](OC)[C@@H](C)C(=O)N[C@H](CCc1ccc(S(=O)(=O)O)cc1)Cc1ccccc1)OC)N(C)C(=O)[C@@H](NC(=O)[C@@H](NC)C(C)C)C(C)C]

58 mg of Intermediate 186 were dissolved in 4.1 ml of dichloromethane, 0.41 ml of trifluoroacetic acid was added and the mixture was stirred at RT for 30 min After concentration under reduced pressure, the crude product was purified by prep. HPLC.

50 mg (90% purity, 85% of theory) of the title compound were obtained.

LC-MS (Method 1): Rₜ=0.87 min; MS (ESIpos): m/z=873 (M+H)⁺

### Intermediate 188

### N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide

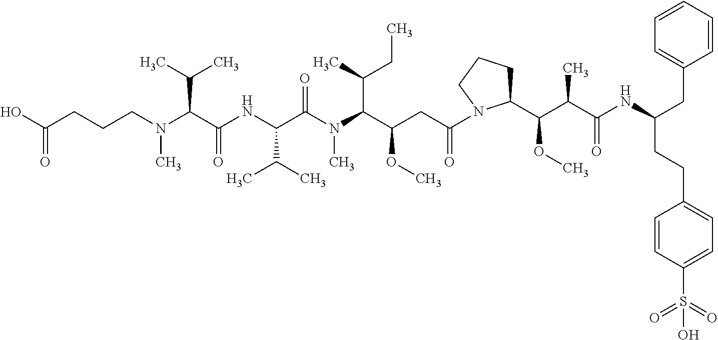 [SMILES: CC[C@H](C)[C@@H]([C@@H](CC(=O)N1CCC[C@H]1[C@H](OC)[C@@H](C)C(=O)N[C@H](CCc1ccc(S(=O)(=O)O)cc1)Cc1ccccc1)OC)N(C)C(=O)[C@@H](NC(=O)[C@H](C(C)C)N(C)CCCC(=O)O)C(C)C]

171 μl (0.26 mmol) of a 15% aqueous succinaldehyde solution were initially charged in 2.5 ml of methanol and admixed with 50 mg (0.05 mmol) of N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide trifluoroacetate and 11.6 μl (0.2 mmol) of acetic acid. The reaction mixture was stirred for 5 min at RT and then 30 μl (0.24 mmol) of borane-pyridine complex were added. After stirring for 24 hours, a further equivalent of borane-pyridine complex was added and the mixture was stirred for a further 2 h. The reaction mixture was then purified by prep. HPLC.

40 mg (90% purity, 66% of theory) of the title compound were obtained.

LC-MS (Method 1): Rₜ=0.91 min; MS (ESIpos): m/z=959 (M+H)⁺

### Intermediate 189

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide

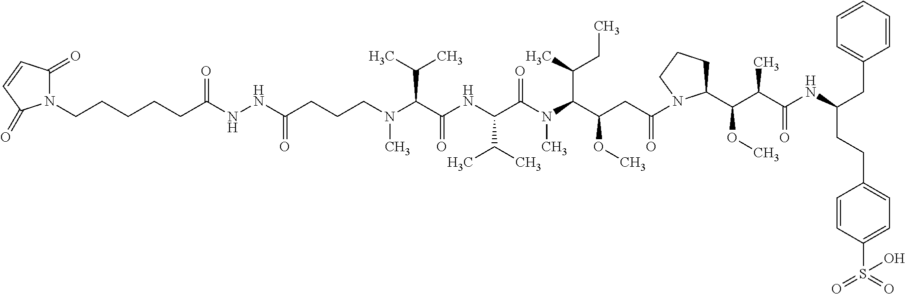 [SMILES: CC[C@H](C)[C@@H]([C@@H](CC(=O)N1CCC[C@H]1[C@H](OC)[C@@H](C)C(=O)N[C@H](CCc1ccc(S(=O)(=O)O)cc1)Cc1ccccc1)OC)N(C)C(=O)[C@@H](NC(=O)[C@H](C(C)C)N(C)CCCC(=O)NNC(=O)CCCCCN1C(=O)C=CC1=O)C(C)C]

10 mg (9.3 μmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide, 3.5 mg (10.3 μmol) of 6-(2,5-dioxo-2,5-dihydro-1H-pyrrol-1-yl)hexanehydrazide trifluoroacetate, 4.3 mg (11.2 μmol) of HATU and 4.9 μl (28 μmol) of Hünig's base were stirred in 1 ml of DMF at RT for 20 h. Subsequently, the reaction mixture was purified by prep. HPLC.

4.2 mg (92% purity, 33% of theory) of the title compound were obtained.

LC-MS (Method 1): Rₜ=0.91 min; MS (ESIpos): m/z=1166 (M+H)⁺

### Intermediate 190

### N-{4-[(2,5-dioxopyrrolidin-1-yl)oxy]-4-oxobutyl}-N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide

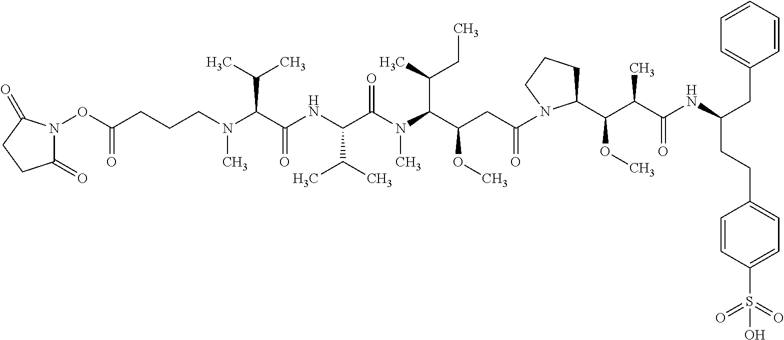 [SMILES: CC[C@H](C)[C@@H]([C@@H](CC(=O)N1CCC[C@H]1[C@H](OC)[C@@H](C)C(=O)N[C@H](CCc1ccc(S(=O)(=O)O)cc1)Cc1ccccc1)OC)N(C)C(=O)[C@@H](NC(=O)[C@H](C(C)C)N(C)CCCC(=O)ON1C(=O)CCC1=O)C(C)C]

To a solution of 10 mg (9.3 μmol) of N-(3-carboxypropyl)-N-methyl-L-valyl-N-[(3R,4S,5S)-3-methoxy-1-{(2S)-2-[(1R,2R)-1-methoxy-2-methyl-3-oxo-3-{[(2R)-1-phenyl-4-(4-sulphophenyl)butan-2-yl]amino}propyl]pyrrolidin-1-yl}-5-methyl-1-oxoheptan-4-yl]-N-methyl-L-valinamide in 2.5 ml of DMF were added 10.7 mg (93 μmol) of N-hydroxysuccinimide, 10.6 mg (28 μmol) of EDCI and 0.12 mg (0.9 μmol) of DMAP. The reaction mixture was stirred at RT for 20 h and then purified by prep. HPLC.

3.8 mg (72% purity, 25% of theory) of the title compound were obtained.

LC-MS (Method 1): Rₜ=0.90 min; MS (ESIpos): m/z=1055 (M+H)⁺

### Intermediate 191

### (2R,3R)—N-[(2S)-3-(1H-indol-3-yl)-1-(1,2-oxazinan-2-yl)-1-oxopropan-2-yl]-3-methoxy-2-methyl-3-[(2S)-pyrrolidin-2-yl]propanamide trifluoroacetate

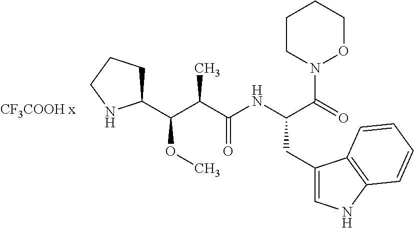 [SMILES: CO[C@@H]([C@@H]1CCCN1)[C@@H](C)C(=O)N[C@@H](Cc1c[nH]c2ccccc12)C(=O)N1CCCCO1]

The title compound was prepared in analogy to the synthesis of Intermediate 7 over two stages from Starting Compound 1 and (2S)-2-amino-3-(1H-indol-3-yl)-1-(1,2-oxazinan-2-yl)propan-1-one trifluoroacetate (Intermediate 99).

Yield over 2 stages: 62 mg (67% of theory)

HPLC (Method 6): Rₜ=1.65 min;

LC-MS (Method 1): Rₜ=0.7 min; MS (ESIpos): m/z=443 (M+H)⁺.

### Intermediate 192

### N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

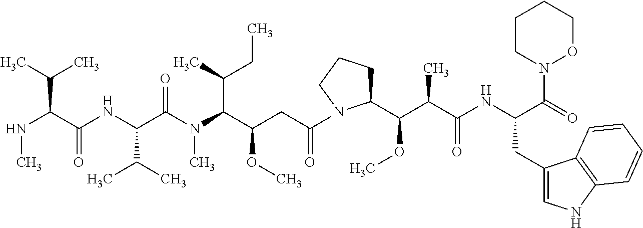 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@@H](NC)C(C)C)C(C)C]

1015 mg (1.59 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were taken up in 50 ml of DMF, admixed with 654 mg (2.39 mmol) of 2-bromo-1-ethylpyridinium tetrafluoroborate (BEP) and 2.8 ml of N,N-diisopropylethylamine, and stirred at RT for 10 min. Then 1083 mg (1.75 mmol) of (2R,3R)—N-[(2S)-3-(1H-indol-3-yl)-1-(1,2-oxazinan-2-yl)-1-oxopropan-2-yl]-3-methoxy-2-methyl-3-[(2S)-pyrrolidin-2-yl]propanamide trifluoroacetate (Intermediate 191) were added and then the mixture was treated in an ultrasound bath at RT for 30 min. The reaction mixture was then concentrated under reduced pressure and the residue was taken up in 300 ml of ethyl acetate. The organic phase was washed successively with 5% aqueous citric acid solution and 5% aqueous sodium hydrogencarbonate solution, dried over magnesium sulphate, filtered and concentrated. The crude product thus obtained (1684 mg), without further purification, was taken up in 20 ml of acetonitrile, 2 ml of piperidine were added and the reaction mixture was then stirred at RT for 10 min. Then the mixture was concentrated under reduced pressure and the residue was admixed with diethyl ether. The solvent was concentrated by evaporation again and the residue was purified by flash chromatography on silica gel (eluent: 15:1:0.1->15:2:0.2 dichloromethane/methanol/17% aqueous ammonia solution). The corresponding fractions were combined, the solvent was removed under reduced pressure and the residue was lyophilized from acetonitrile/water. Thus, 895 mg (67% over 2 stages) of the title compound were obtained.

HPLC (Method 12): Rₜ=1.8 min;

LC-MS (Method 1): Rₜ=0.84 min; MS (ESIpos): m/z=840 (M+H)⁺.

¹H NMR (500 MHz, DMSO-d₆): δ=10.8 (d, 1H), 8.3 and 8.05 (2d, 1H), 8.0 (d, 1H), 7.5 (m, 1H), 7.3 (m, 1H), 7.15 and 7.08 (2s, 1H) 7.05-6.9 (m, 2H), 5.12 and 4.95 (2m, 1H), 4.65 (m, 1H), 4.55 (m, 1H), 4.1-3.8 (m, 4H), 3.75 (d, 1H), 3.23, 3.18, 3.17, 3.12, 2.95 and 2.88 (6s, 9H), 3.1-3.0 and 2.85 (2m, 2H), 2.65 (d, 1H), 2.4-2.2 (m, 3H), 2.15 (m, 3H), 1.95 (br. m, 2H), 1.85-0.8 (br. m, 11H), 1.08 and 1.04 (2d, 3H), 0.9-0.75 (m, 15H), 0.75-0.65 (dd, 3H) [further signals hidden under H₂O peak].

### Intermediate 193

### N-(3-carboxypropyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

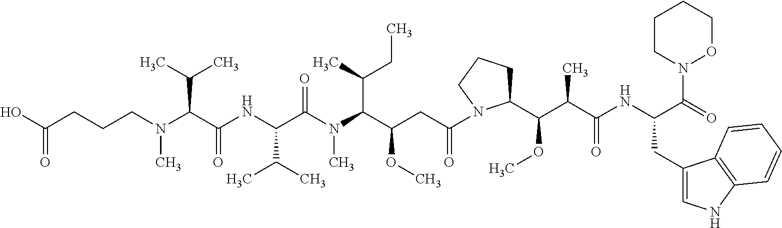 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCC(=O)O)C(C)C]

50 mg (0.052 mmol) of N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 192) and 204 μl einer of a 15% aqueous solution of 4-oxobutanoic acid were combined in 2 ml of methanol and admixed with 23.4 mg (0.252 mmol) of borane-pyridine complex and 6 μl of acetic acid. The reaction mixture was stirred at RT overnight. This was followed by concentration under reduced pressure, and the residue was purified by means of preparative HPLC. After concentration of the corresponding fractions, 38 mg (78% of theory) of the title compound were obtained.

HPLC (Method 5): Rₜ=1.7 min;

LC-MS (Method 9): Rₜ=4.7 min; MS (ESIpos): m/z=926 (M+H)⁺.

### Intermediate 194

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

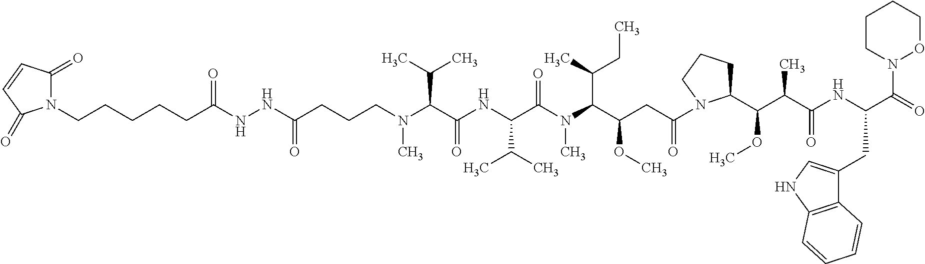 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCC(=O)NNC(=O)CCCCCN1C(=O)C=CC1=O)C(C)C]

This compound was prepared in analogy to the synthesis described in Intermediate 157 from 10 mg (11 μmol) of N-(3-carboxypropyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide and commercially available 6-(2,5-dioxo-2,5-dihydro-1H-pyrrol-1-yl)hexanehydrazide.

Yield: 4.4 mg (35% of theory)

HPLC (Method 5): Rₜ=1.8 min;

LC-MS (Method 1): Rₜ=0.90 min; MS (ESIpos): m/z=1133 (M+H)⁺.

### Intermediate 195

### N-[6-(2,5-dioxo-2,5-dihydro-1H-pyrrol-1-yl)hexyl]-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide

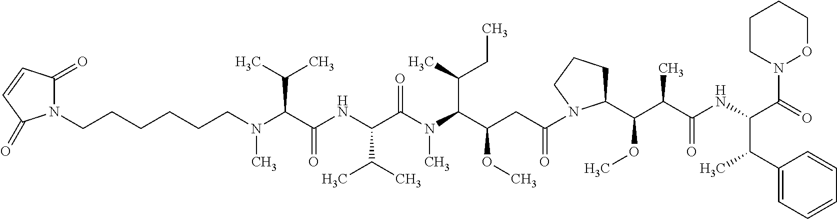 [SMILES: CC[C@H](C)[C@@H]([C@@H](CC(=O)N1CCC[C@H]1[C@H](OC)[C@@H](C)C(=O)N[C@H](C(=O)N1CCCCO1)[C@@H](C)c1ccccc1)OC)N(C)C(=O)[C@@H](NC(=O)[C@H](C(C)C)N(C)CCCCCCN1C(=O)C=CC1=O)C(C)C]

This compound was prepared in analogy to Intermediate 166, proceeding from 9 mg (0.010 mmol) of Intermediate 170.

Yield: 1.1 mg (10% of theory)

HPLC (Method 12): Rₜ=2.0 min;

LC-MS (Method 1): Rₜ=0.99 min; MS (ESIpos): m/z=994 (M+H)⁺.

### Intermediate 196

### (2S)-2-amino-1-(2-oxa-3-azabicyclo[2.2.2]oct-5-en-3-yl)-3-phenylpropan-1-one trifluoroacetate

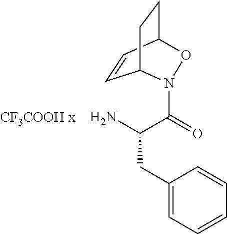 [SMILES: N[C@@H](Cc1ccccc1)C(=O)N1OC2C=CC1CC2]

41 mg (0.37 mmol) of 2,5-dioxopyrrolidin-1-yl N-(tert-butoxycarbonyl)-L-phenylalaninate were taken up in 10 ml of DMF and admixed with 149 mg (0.41 mmol) of 2-oxa-3-azabicyclo[2.2.2]oct-5-ene (Starting Compound 6) and 72 μl (0.41 mmol) of N,N-diisopropylethylamine. The mixture was stirred at RT for 1 h. The solvent was removed under reduced pressure, and the residue was taken up in ethyl acetate and extracted by shaking with 5% aqueous citric acid solution and then with 5% aqueous sodium hydrogencarbonate solution. The organic phase was concentrated and the residue was purified by flash chromatography on silica gel with 10:1 toluene/ethanol as the eluent. The corresponding fractions were combined and the solvent was removed under reduced pressure. After the residue had been dried under high vacuum, 69 mg (47% of theory) of the Boc-protected intermediate tert-butyl (2S)-1-(2-oxa-3-azabicyclo[2.2.2]oct-5-en-3-yl)-1-oxo-3-phenylpropan-2-yl carbamate were thus obtained as a diastereomer mixture.

LC-MS (Method 1): Rₜ=1.1 min; MS (ESIpos): m/z=359 (M+H)⁺.

64 mg (0.18 mmol) of this intermediate were taken up in 10 ml of dichloromethane, 1 ml of trifluoroacetic acid was added, and the mixture was stirred at RT for 30 min. This was followed by concentration under reduced pressure and lyophilization of the remaining residue from water/dioxane. In this way, 66 mg (quant.) of the title compound were obtained as a foam.

HPLC (Method 6): Rₜ=1.45 min;

LC-MS (Method 3): Rₜ=1.12 min; MS (ESIpos): m/z=259 (M+H)⁺.

### Intermediate 197

### (2R,3R)-3-methoxy-2-methyl-N-[(2S)-1-(2-oxa-3-azabicyclo[2.2.2]oct-5-en-3-yl)-1-oxo-3-phenylpropan-2-yl]-3-[(2S)-pyrrolidin-2-yl]propanamide trifluoroacetate

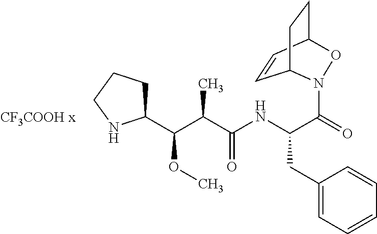 [SMILES: CO[C@@H]([C@@H]1CCCN1)[C@@H](C)C(=O)N[C@@H](Cc1ccccc1)C(=O)N1OC2C=CC1CC2]

First, (2R,3R)-3-[(2S)-1-(tert-butoxycarbonyl)pyrrolidin-2-yl]-3-methoxy-2-methylpropanoic acid (Starting Compound 1) was released from 83 mg (0.18 mmol) of its dicyclohexylamine salt by taking it up in ethyl acetate and extractive shaking with 5% aqueous potassium hydrogensulphate solution. The organic phase was dried over magnesium sulphate, filtered and concentrated. The residue was taken up in 10 ml of DMF and admixed successively with 66 mg (0.18 mmol) of (2S)-2-amino-1-(2-oxa-3-azabicyclo[2.2.2]oct-5-en-3-yl)-3-phenylpropan-1-one trifluoroacetate (Intermediate 196), 101 mg (0.266 mmol) of O-(7-azabenzotriazol-1-yl)-N,N,N′,N′-tetramethyluronium hexafluorophosphate (HATU) and 93 μl (0.53 mmol) of N,N-diisopropylethylamine. The mixture was stirred at RT for 30 min. The reaction mixture was then concentrated and the residue was purified by preparative HPLC. This gave 52 mg (56% of theory) of the Boc-protected intermediate tert-butyl (2S)-2-[(1R,2R)-1-methoxy-2-methyl-3-{[(2S)-1-(2-oxa-3-azabicyclo[2.2.2]oct-5-en-3-yl)-1-oxo-3-phenylpropan-2-yl]amino}-3-oxopropyl]pyrrolidine-1-carboxylate.

HPLC (Method 6): Rₜ=2.13 min;

LC-MS (Method 1): Rₜ=1.13 min; MS (ESIpos): m/z=528 (M+H)⁺.

52 mg (0.1 mmol) of this intermediate were taken up in 10 ml of dichloromethane, 1 ml of trifluoroacetic acid was added, and the mixture was stirred at RT for 20 min. This was followed by concentration under reduced pressure and stirring of the remaining residue with 20 ml of diethyl ether. After 10 min, the mixture was filtered and the filter residue was dried under high vacuum. In this way, 39 mg (72% of theory) of the title compound were obtained.

HPLC (Method 6): Rₜ=1.62 min;

LC-MS (Method 1): Rₜ=0.68 min; MS (ESIpos): m/z=428 (M+H)⁺.

### Intermediate 198

### N-methyl-L-valyl-N-[(3R,4S,5S)-3-methoxy-1-{(2S)-2-[(1R,2R)-1-methoxy-2-methyl-3-{[(2S)-1-(2-oxa-3-azabicyclo[2.2.2]oct-5-en-3-yl)-1-oxo-3-phenylpropan-2-yl]amino}-3-oxopropyl]pyrrolidin-1-yl}-5-methyl-1-oxoheptan-4-yl]-N-methyl-L-valinamide trifluoro acetate

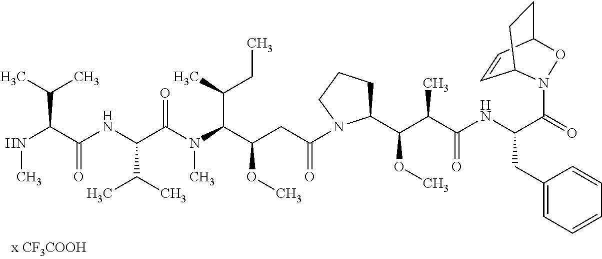 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1OC2C=CC1CC2)OC)N(C)C(=O)[C@@H](NC(=O)[C@@H](NC)C(C)C)C(C)C]

44.5 mg (0.071 mmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) were taken up in 10 ml of DMF and admixed successively with 38.6 mg (0.071 mmol) of (2R,3R)-3-methoxy-2-methyl-N-[(2S)-1-(2-oxa-3-azabicyclo[2.2.2]oct-5-en-3-yl)-1-oxo-3-phenylpropan-2-yl]-3-[(2S)-pyrrolidin-2-yl]propanamide trifluoroacetate (Intermediate 197), 32.5 mg (0.086 mmol) of O-(7-azabenzotriazol-1-yl)-N,N,N′,N′-tetramethyluronium hexafluorophosphate (HATU) and 41 μl (0.235 mmol) of N,N-diisopropylethylamine. The mixture was stirred at RT for 1 h. The reaction mixture was then concentrated under reduced pressure and the residue was taken up in ethyl acetate. The organic phase was washed successively with 5% aqueous citric acid solution and 5% aqueous sodium hydrogencarbonate solution, dried over magnesium sulphate, filtered and concentrated. This gave 73 mg (98% of theory) of the Fmoc-protected intermediate N-[(9H-fluoren-9-ylmethoxy)carbonyl]-N-methyl-L-valyl-N-[(3R,4S,5S)-3-methoxy-1-{(2S)-2-[(1R,2R)-1-methoxy-2-methyl-3-{[(2S)-1-(2-oxa-3-azabicyclo[2.2.2]oct-5-en-3-yl)-1-oxo-3-phenylpropan-2-yl]amino}-3-oxopropyl]pyrrolidin-1-yl}-5-methyl-1-oxoheptan-4-yl]-N-methyl-L-valinamide

HPLC (Method 6): Rₜ=2.78 min;

LC-MS (Method 3): Rₜ=2.96 min; MS (ESIpos): m/z=1047 (M+H)⁺.

73 mg (0.071 mmol) of this intermediate were dissolved in 5 ml of DMF. After 0.5 ml of piperidine had been added, the reaction mixture was stirred at RT for 10 min. This was followed by concentration under reduced pressure, and the residue was digested repeatedly with diethyl ether. After the diethyl ether had been decanted off, the residue was purified by preparative HPLC (eluent: acetonitrile 0.1% aq. TFA). 16 mg (26% of theory) of the title compound were obtained as a foam.

HPLC (Method 6): Rₜ=1.94 min;

LC-MS (Method 3): Rₜ=1.71 min; MS (ESIpos): m/z=825 (M+H)⁺

¹H NMR (400 MHz, DMSO-d₆): δ=8.9-8.6 (m, 3H), 8.4, 8.3, 8.1 and 8.0 (4d, 1H), 7.3-7.1 (m, 5H), 6.7-6.5 (m, 2H), 5.2-4.8 (m, 3H), 4.75-4.55 (m, 3H), 4.05-3.95 (m, 1H), 3.7-3.4 (m, 4H), 3.22, 3.17, 3.15, 3.05, 3.02 and 2.95 (6s, 9H), 3.0 and 2.7 (2 br. m, 2H), 2.46 (m, 3H), 2.4-1.2 (br. m, 13H), 1.1-0.85 (m, 18H), 0.75 (m, 3H) [further signals hidden under H₂O peak].

### Intermediate 199

### N-(4-{2-[6-(2,5-dioxo-2,5-dihydro-1H-pyrrol-1-yl)hexanoyl]hydrazino}-4-oxobutyl)-N-methyl-L-valyl-N-[(3R,4S,5S)-3-methoxy-1-{(2S)-2-[(1R,2R)-1-methoxy-2-methyl-3-{[(2S)-1-(2-oxa-3-azabicyclo[2.2.2]oct-5-en-3-yl)-1-oxo-3-phenylpropan-2-yl]amino}-3-oxopropyl]pyrrolidin-1-yl}-5-methyl-1-oxoheptan-4-yl]-N-methyl-L-valinamide

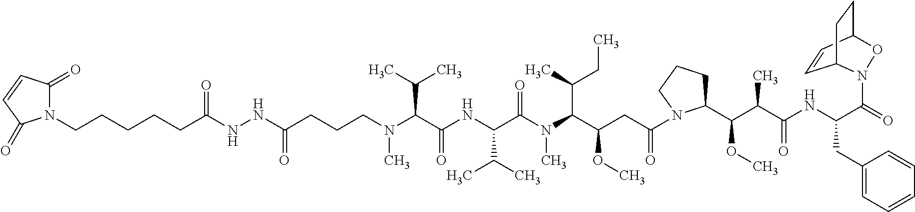 [SMILES: CC[C@H](C)[C@@H]([C@@H](CC(=O)N1CCC[C@H]1[C@H](OC)[C@@H](C)C(=O)N[C@@H](Cc1ccccc1)C(=O)N1OC2C=CC1CC2)OC)N(C)C(=O)[C@@H](NC(=O)[C@H](C(C)C)N(C)CCCC(=O)NNC(=O)CCCCCN1C(=O)C=CC1=O)C(C)C]

The title compound was prepared in analogy to Intermediates 193 and 194, proceeding from 23 mg (24 μmol) of N-methyl-L-valyl-N-[(3R,4S,5S)-3-methoxy-1-{(2S)-2-[(1R,2R)-1-methoxy-2-methyl-3-{[(2S)-1-(2-oxa-3-azabicyclo[2.2.2]oct-5-en-3-yl)-1-oxo-3-phenylpropan-2-yl]amino}-3-oxopropyl]pyrrolidin-1-yl}-5-methyl-1-oxoheptan-4-yl]-N-methyl-L-valinamide trifluoroacetate (Intermediate 198).

HPLC (Method 12): Rt=1.9 min;

LC-MS (Method 2): Rt=2.1 min; MS (ESIpos): m/z=1118 (M+H)+.

### Intermediate 200

### N-[2-(2-{2-[2-(2,5-dioxo-2,5-dihydro-1H-pyrrol-1-yl)ethoxy]ethoxy}ethoxy)ethyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

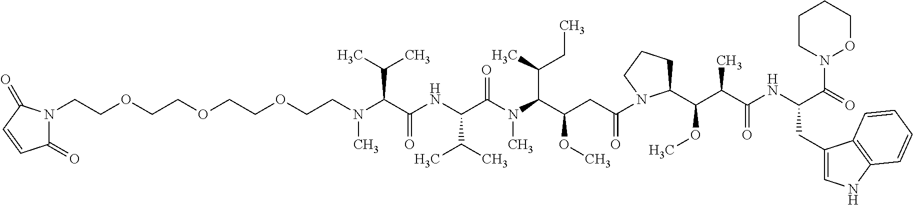 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCOCCOCCOCCN1C(=O)C=CC1=O)C(C)C]

The preparation was effected in analogy to Intermediates 174 and 175, commencing with the reductive alkylation of Intermediate 192 with Intermediate 172, subsequent deprotection and formation of the maleimide.

HPLC (Method 12): Rt=1.9 min;

LC-MS (Method 1): Rt=0.86 min; MS (ESIpos): m/z=1025 (M+H)+.

### Intermediate 201

### N-{6-[(bromoacetyl)amino]hexyl}-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

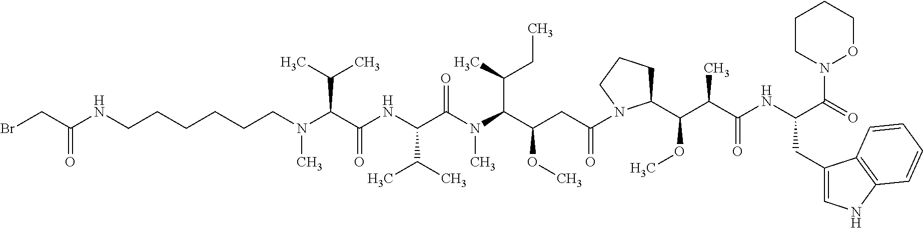 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCCNC(=O)CBr)C(C)C]

22 mg (0.023 mmol) of N-(6-aminohexyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 101) were dissolved in 9.5 ml of THF and admixed at 0° C. with 4.2 μl of triethylamine. A solution of bromoacetyl chloride in THF was added dropwise and the reaction mixture was stirred at 0° C. for 30 min. The reaction mixture was concentrated and the residue was purified by preparative HPLC. Thus, 6.9 mg (26% of theory) of the title compound were obtained as a foam.

HPLC (Method 5): Rₜ=1.8 min;

LC-MS (Method 11): Rₜ=0.9 min; MS (ESIpos): m/z=1059 and 1061 (M+H)⁺.

### Intermediate 202

### N-{2-[2-(2-{3-[(2,5-dioxopyrrolidin-1-yl)oxy]-3-oxopropoxy}ethoxy)ethoxy]ethyl}-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

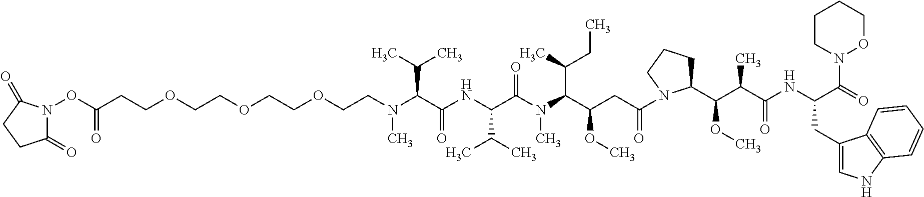 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCOCCOCCOCCC(=O)ON1C(=O)CCC1=O)C(C)C]

The preparation was at first effected in analogy to Intermediate 168, commencing with the reductive alkylation of Intermediate 192 with Intermediate 167 and subsequent hydrogenolytic cleavage of the benzyl ester of N-(2-{2-[2-(2-carboxyethoxy)ethoxy]ethoxy}ethyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide.

13 mg (10 μmol) of this intermediate were dissolved in 5 ml of DMF and admixed with 2.1 mg (20 mmol) of 1-hydroxypyrrolidine-2,5-dione, 6.5 μl of N,N-diisopropylethylamine and 7.1 mg (0.02 mmol) of HATU. The reaction mixture was stirred at RT overnight and then concentrated under reduced pressure. The remaining residue was purified by means of preparative HPLC. After lyophilization from acetonitrile/water, 9.2 mg (62% of theory) of the title compound were obtained.

HPLC (Method 12): Rₜ=2.0 min;

LC-MS (Method 2): Rₜ=2.1 min; MS (ESIpos): m/z=1141 (M+H)⁺.

### Intermediate 203

### tert-butyl 6-hydrazino-6-oxohexyl carbamate

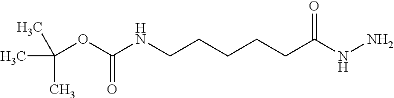 [SMILES: CC(C)(C)OC(=O)NCCCCCC(=O)NN]

This compound was prepared by standard peptide chemistry methods, by coupling of 6-[(tert-butoxycarbonyl)amino]hexanoic acid with benzyl hydrazinecarboxylate in the presence of EDCI and HOBT, and subsequent hydrogenolytic cleavage of the benzyloxycarbonyl protecting group.

LC-MS (Method 11): Rₜ=0.59 min; MS (ESIpos): m/z=246 (M+H)⁺.

### Intermediate 204

### N-{4-[2-(6-aminohexanoyl)hydrazino]-4-oxobutyl}-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

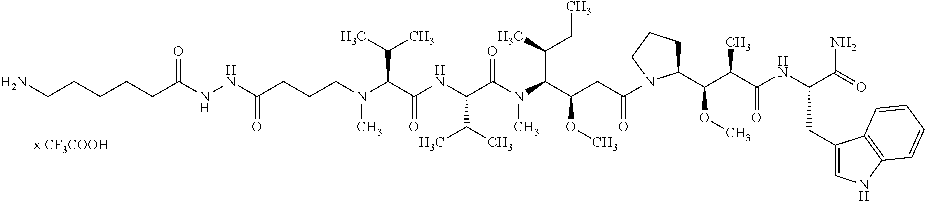 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)NNC(=O)CCCCCN)C(C)C]

146 mg (50 μmol) of (N-(3-carboxypropyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide were dissolved in 5 ml of DMF and then admixed with 30.6 mg (80 μmol) of O-(7-azabenzotriazol-1-yl)-N,N,N′,N′-tetramethyluronium hexafluorophosphate, 19 μl of N,N-diisopropylethylamine and with 22.4 mg (60 μmol) of tert-butyl 6-hydrazino-6-oxohexyl carbamate. The reaction mixture was stirred at RT for 1.5 h. This was followed by concentration under high vacuum and purification of the remaining residue by means of preparative HPLC. Thus, 43 mg (68% of theory) of the protected intermediate were obtained, which were then taken up in 10 ml of dichloromethane and deprotected with 1 ml of trifluoroacetic acid. The reaction mixture was concentrated and the residue was stirred with dichloromethane, and the solvent was removed again under reduced pressure. Thus, 45 mg (68% of theory over 2 stages) of the title compound were obtained.

HPLC (Method 12): Rₜ=1.6 min;

LC-MS (Method 11): Rₜ=0.66 min; MS (ESIpos): m/z=983 (M+H)⁺.

### Intermediate 205

### N-(4-{2-[6-({[2-(2,5-dioxo-2,5-dihydro-1H-pyrrol-1-yl)ethyl]carbamoyl}amino)hexanoyl]hydrazino}-4-oxobutyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

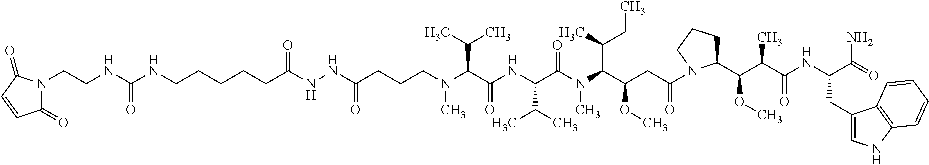 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(N)=O)OC)N(C)C(=O)[C@@H](NC(=O)[C@H](C(C)C)N(C)CCCC(=O)NNC(=O)CCCCCNC(=O)NCCN1C(=O)C=CC1=O)C(C)C]

This compound was prepared in analogy to Intermediate 114, proceeding from Intermediates 50 and 204.

Yield: 4 mg (78% of theory)

HPLC (Method 12): Rₜ=1.7 min;

LC-MS (Method 11): Rₜ=0.73 min; MS (ESIpos): m/z=1149 (M+H)⁺.

### Intermediate 206

### N-(6-{[3-({3-[(2,5-dioxopyrrolidin-1-yl)oxy]-3-oxopropyl}disulphanyl)propanoyl]amino}hexyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

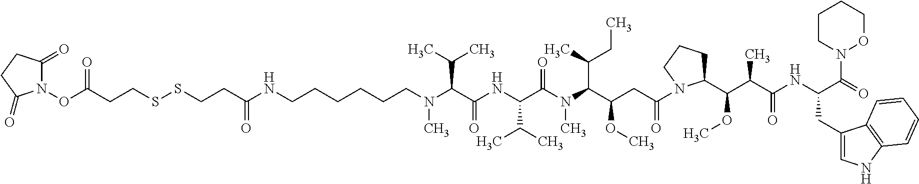 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCCNC(=O)CCSSCCC(=O)ON1C(=O)CCC1=O)C(C)C]

8 mg (10 μmol) of Intermediate 101 were dissolved in 2 ml of DMF and admixed with 8.6 mg (20 μmol) of 1,1′-{-disulphanediylbis[(1-oxopropane-3,1-diyl)oxy]}dipyrrolidine-2,5-dione and 3.7 μl of N,N-diisopropylethylamine. The reaction mixture was stirred at RT for 2 h and then the solvent was evaporated off under reduced pressure and the residue was purified by preparative HPLC. 7.2 mg (68% of theory) of the title compound were obtained.

HPLC (Method 5): Rₜ=1.9 min;

LC-MS (Method 11): Rₜ=0.94 min; MS (ESIpos): m/z=615 [½ (M+2H⁺]

### Intermediate 207

### (1S,2R)-1-amino-2-phenylcyclopropanecarboxylic acid trifluoroacetate

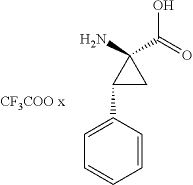 [SMILES: N[C@@]1(C(=O)O)C[C@@H]1c1ccccc1]

The title compound was obtained in quantitative yield by deprotecting 210 mg (0.76 mmol) of commercially available (1S,2R)-1-[(tert-butoxycarbonyl)amino]-2-phenylcyclopropanecarboxylic acid with trifluoroacetic acid.

LC-MS (Method 1): Rₜ=0.23 min; MS (ESIpos): m/z=178 (M+H)⁺.

### Intermediate 208

### 9H-fluoren-9-ylmethyl 6-oxohexyl carbamate

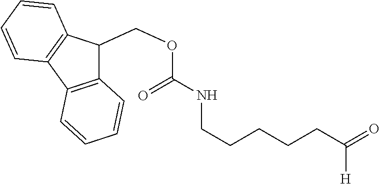 [SMILES: O=CCCCCCNC(=O)OCC1c2ccccc2-c2ccccc21]

The title compound was prepared from 1 g (2.95 mmol) of commercially available 9H-fluoren-9-ylmethyl 6-hydroxyhexyl carbamate under standard conditions, by oxidation with sulphur trioxide-pyridine complex. 840 mg (85% of theory) of the title compound were obtained.

HPLC (Method 12): Rₜ=2.0 min;

LC-MS (Method 1): Rₜ=1.1 min; MS (ESIpos): m/z=338 (M+H)⁺.

### Intermediate 209

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(1S,2R)-1-carboxy-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide

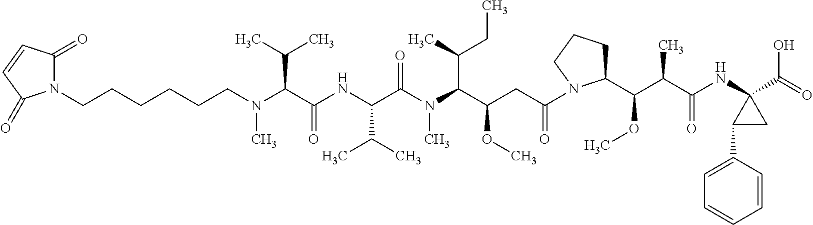 [SMILES: CC[C@H](C)[C@@H]([C@@H](CC(=O)N1CCC[C@H]1[C@H](OC)[C@@H](C)C(=O)N[C@@]1(C(=O)O)C[C@@H]1c1ccccc1)OC)N(C)C(=O)[C@@H](NC(=O)[C@H](C(C)C)N(C)CCCCCCN1C(=O)C=CC1=O)C(C)C]

First, in analogy to the synthesis described in Intermediate 75, by coupling of N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and (1S,2R)-1-amino-2-phenylcyclopropanecarboxylic acid trifluoroacetate (Intermediate 207) in the presence of O-(7-azabenzotriazol-1-yl)-N,N,N′,N′-tetramethyluronium hexafluorophosphate and subsequent detachment of the Boc protecting group by means of trifluoroacetic acid, the amine compound N-methyl-L-valyl-N-[(3R,4S,5S)-1-{(2S)-2-[(1R,2R)-3-{[(1S,2R)-1-carboxy-2-phenylcyclopropyl]amino}-1-methoxy-2-methyl-3-oxopropyl]pyrrolidin-1-yl}-3-methoxy-5-methyl-1-oxoheptan-4-yl]-N-methyl-L-valinamide was prepared as the trifluoroacetate.

To 22 mg (0.026 mmol) of this compound in 10 ml of methanol were then added 17 mg (0.05 mmol) of 9H-fluoren-9-ylmethyl 6-oxohexyl carbamate (Intermediate 208) and 2.3 mg of acetic acid, and also 11.4 mg (0.12 mmol) of borane-pyridine complex. The reaction mixture was stirred at RT overnight. Then the same amounts of borane-pyridine complex and acetic acid, and also 8 mg of fluoren-9-ylmethyl 6-oxohexyl carbamate, were added once again and the reaction mixture was stirred at RT for a further 24 h. This was followed by concentration under reduced pressure, and the residue was purified by means of preparative HPLC. After concentration of the corresponding fractions, the product was used immediately in the next stage. 33 mg of the still contaminated intermediate were taken up in 5 ml of DMF, and 1 ml of piperidine was added. After stirring at RT for 15 min, the reaction mixture was concentrated and the resulting residue was purified by preparative HPLC. Thus, 11 mg (55% of theory over 2 stages) of the aminocarboxylic acid intermediate were obtained.

HPLC (Method 12): Rₜ=1.7 min;

LC-MS (Method 11): Rₜ=0.7 min; MS (ESIpos): m/z=843 (M+H)⁺.

6 mg (7.12 μmol) of this intermediate were taken up in 1 ml of dioxane and then admixed with 6.6 mg (42.7 μmol) of methyl 2,5-dioxo-2,5-dihydro-1H-pyrrole-1-carboxylate and with 5 μl of saturated aqueous sodium hydrogencarbonate solution. The reaction mixture was stirred at RT for 1 h. Then another 3 portions each of 50 μl of the saturated aqueous sodium hydrogencarbonate solution were added and the reaction mixture was stirred at RT for a further 30 min. Then the reaction mixture was acidified to pH 2 with trifluoroacetic acid and subsequently concentrated under reduced pressure. The remaining residue was purified by means of preparative HPLC. After lyophilization from acetonitrile/water, 4 mg (60% of theory) of the title compound were obtained as a foam.

HPLC (Method 12): Rₜ=1.9 min;

LC-MS (Method 11): Rₜ=0.88 min; MS (ESIpos): m/z=923 (M+H)⁺.

### Intermediate 210

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

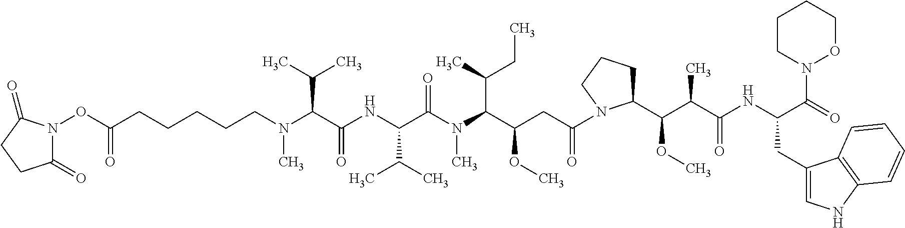 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCC(=O)ON1C(=O)CCC1=O)C(C)C]

First, 6-oxohexanoic acid was prepared by a literature method (J. Org. Chem. 58, 1993, 2196-2200). 80 mg (0.08 mmol) of N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 192) and 65.4 mg (0.5 mmol) of 6-oxohexanoic acid were combined in 9 ml of methanol and admixed with 10 μl of acetic acid and 37.4 mg (0.4 mmol) of borane-pyridine complex. The reaction mixture was stirred at RT overnight. This was followed by concentration under reduced pressure, and the residue was taken up in 1:1 acetonitrile/water and adjusted to pH 2 with trifluoroacetic acid. The reaction mixture was concentrated again and the residue was purified by means of preparative HPLC. After concentration of the corresponding fractions, 70 mg (86% of theory) of N-(5-carboxypentyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide were obtained as the trifluoroacetate.

HPLC (Method 12): Rₜ=1.9 min;

LC-MS (Method 1): Rₜ=0.87 min; MS (ESIpos): m/z=955 (M+H)⁺.

¹H NMR (500 MHz, DMSO-d₆, characteristic signals): δ=12.0 (br. M, 1H), 10.8 (s, 1H), 9.4 (m, 1H), 8.9 and 8.8 (2d, 1H), 8.3 and 8.02 (2d, 1H), 7.5 (m, 1H), 7.3 (m, 1H), 7.15 and 7.1 (2s, 1H) 7.05-6.9 (m, 2H), 5.12 and 4.95 (2m, 1H), 4.7-4.5 (m, 2H), 4.1-3.8 (m, 4H), 3.75 (d, 1H), 3.25, 3.2, 3.18, 3.13, 2.98 and 2.88 (6s, 9H), 2.8 (m, 3H), 1.08 and 1.04 (2d, 3H), 0.95-0.8 (m, 15H), 0.8-0.65 (dd, 3H).

22 mg (23 μmol) of this intermediate were dissolved in 1.8 ml of dichloromethane and admixed with 13.2 mg (70 μmol) of 1-(3-dimethylaminopropyl)-3-ethylcarbodiimide hydrochloride, 26.5 mg (230 μmol) of 1-hydroxypyrrolidine-2,5-dione and 0.28 mg (2 μmol) of dimethylaminopyridine, and the reaction mixture was stirred at RT for 2 h. Subsequently, the reaction mixture was concentrated under reduced pressure and the remaining residue was purified by means of preparative HPLC. After lyophilization from acetonitrile/water, 21.3 mg (88% of theory) of the title compound were obtained.

HPLC (Method 12): Rₜ=1.9 min;

LC-MS (Method 1): Rₜ=0.94 min; MS (ESIpos): m/z=1052 (M+H)⁺.

### Intermediate 211

### N-{6-[(2,5-dioxopyrrolidin-1-yl)oxy]-6-oxohexyl}-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide

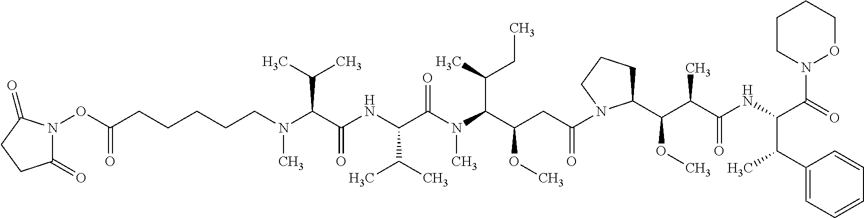 [SMILES: CC[C@H](C)[C@@H]([C@@H](CC(=O)N1CCC[C@H]1[C@H](OC)[C@@H](C)C(=O)N[C@H](C(=O)N1CCCCO1)[C@@H](C)c1ccccc1)OC)N(C)C(=O)[C@@H](NC(=O)[C@H](C(C)C)N(C)CCCCCC(=O)ON1C(=O)CCC1=O)C(C)C]

15 mg (20 μmol) of N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidinyl}-5-methyl-1-oxoheptan-4-yl]-N-methyl-L-valinamide trifluoroacetate (Intermediate 15) were reductively alkylated with 6-oxohexanoic acid, in analogy to Intermediate 210.

Yield: 9.2 mg (61% of theory)

HPLC (Method 12): Rₜ=1.9 min;

LC-MS (Method 1): Rₜ=0.87 min; MS (ESIpos): m/z=929 (M+H)⁺.

9 mg (10 μmol) of this intermediate were dissolved in 3 ml of DMF and admixed with 5.6 mg (48 μmol) of 1-hydroxypyrrolidine-2,5-dione, 5 μl of N,N-diisopropylethylamine and 5.5 mg (0.015 mmol) of HATU, and the reaction mixture was treated in an ultrasound bath for 6 h. In the course of this, 5.5 mg of HATU were added every hour. Subsequently, the reaction mixture was concentrated under reduced pressure, and the residue was taken up in acetonitrile/water and adjusted to pH 2 with trifluoroacetic acid. After concentrating again under reduced pressure, the remaining residue was purified by means of preparative HPLC. After lyophilization from acetonitrile/water, 5.8 mg (57% of theory) of the title compound were obtained.

HPLC (Method 12): Rₜ=2.0 min;

LC-MS (Method 1): Rₜ=0.95 min; MS (ESIpos): m/z=1027 (M+H)⁺.

### Intermediate 212

### N-{2-[2-(2-{3-[(2,5-dioxopyrrolidin-1-yl)oxy]-3-oxopropoxy}ethoxy)ethoxy]ethyl}-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide

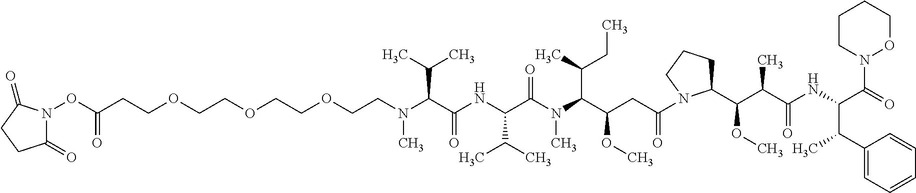 [SMILES: CC[C@H](C)[C@@H]([C@@H](CC(=O)N1CCC[C@H]1[C@H](OC)[C@@H](C)C(=O)N[C@H](C(=O)N1CCCCO1)[C@@H](C)c1ccccc1)OC)N(C)C(=O)[C@@H](NC(=O)[C@H](C(C)C)N(C)CCOCCOCCOCCC(=O)ON1C(=O)CCC1=O)C(C)C]

The preparation was at first effected in analogy to Intermediate 168, commencing with the reductive alkylation of Intermediate 15 with Intermediate 167 and subsequent hydrogenolytic cleavage of the benzyl ester of N-(2-{2-[2-(2-carboxyethoxy)ethoxy]ethoxy}ethyl)-N-methyl-L-valyl-N-[(3R,4S,5S)-3-methoxy-1-{(2S)-2-[(1R,2R)-1-methoxy-2-methyl-3-{[(2S,3S)-1-(1,2-oxazinan-2-yl)-1-oxo-3-phenylbutan-2-yl]amino}-3-oxopropyl]pyrrolidin-1-yl}-5-methyl-1-oxoheptan-4-yl]-N-methyl-L-valinamide

8.4 mg (8 μmol) of this intermediate were dissolved in 3 ml of DMF and admixed with 9.5 mg (80 μmol) of 1-hydroxypyrrolidine-2,5-dione, 10 μl of N,N-diisopropylethylamine and 9.4 mg (25 μmol) of HATU, and the reaction mixture was stirred at RT overnight and then concentrated under reduced pressure. Subsequently, the reaction mixture was concentrated under reduced pressure, and the residue was taken up in acetonitrile/water and adjusted to pH 2 with trifluoroacetic acid. After concentrating again under reduced pressure, the remaining residue was purified by means of preparative HPLC. After lyophilization from acetonitrile/water, 4 mg (32% of theory) of the title compound were obtained.

HPLC (Method 12): Rₜ=2.0 min;

LC-MS (Method 1): Rₜ=0.96 min; MS (ESIpos): m/z=1117 (M+H)⁺.

### Intermediate 213

### N-{6-[(trans-4-{[(2,5-dioxopyrrolidin-1-yl)oxy]carbonyl}cyclohexyl)amino]-6-oxohexyl}-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

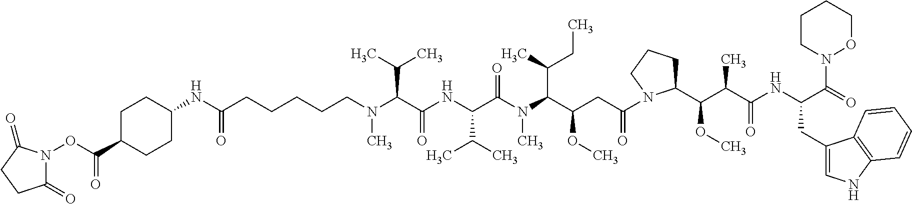 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCC(=O)N[C@H]1CC[C@H](C(=O)ON2C(=O)CCC2=O)CC1)C(C)C]

This compound was prepared in analogy to Intermediate 104, proceeding from N-(5-carboxypentyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide, the synthesis of which was described under Intermediate 210. 9.3 mg of the title compound (37% of theory over 3 stages) were obtained.

HPLC (Method 12): Rₜ=1.9 min;

LC-MS (Method 1): Rₜ=0.9 min; MS (ESIpos): m/z=1177 (M+H)⁺.

### Intermediate 214

### N-{4-[(2,5-dioxopyrrolidin-1-yl)oxy]-4-oxobutyl}-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

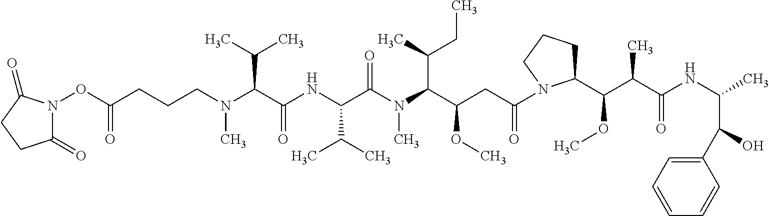 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)CCCC(=O)ON1C(=O)CCC1=O)C(C)C]

This compound was prepared in analogy to Intermediate 210, by conversion of Intermediate 92 to the active ester.

HPLC (Method 5): Rₜ=1.6 min;

LC-MS (Method 11): Rₜ=0.82 min; MS (ESIpos): m/z=901 (M+H)⁺.

### Intermediate 215

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

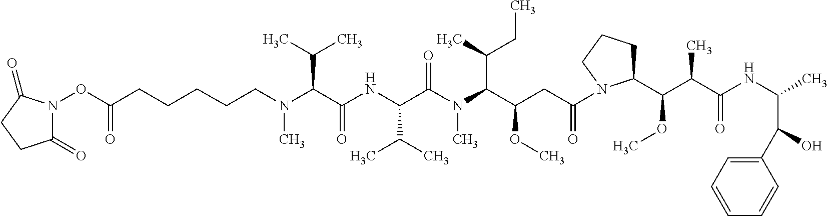 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)CCCCCC(=O)ON1C(=O)CCC1=O)C(C)C]

First, Intermediate 40, in analogy to Intermediate 183, was used with borane-pyridine complex to prepare N-(5-carboxypentyl)-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide. From this compound, in analogy to Intermediate 210, the active ester was then generated. 34 mg (36% of theory over 2 stages) of the title compound were obtained.

HPLC (Method 5): Rₜ=1.6 min;

LC-MS (Method 1): Rₜ=0.85 min; MS (ESIpos): m/z=930 (M+H)⁺.

### Intermediate 216

### N-(4-{[(2,5-dioxopyrrolidin-1-yl)oxy]carbonyl}benzyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

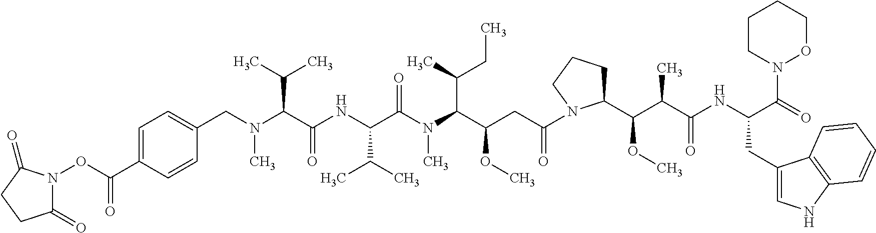 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)Cc1ccc(C(=O)ON2C(=O)CCC2=O)cc1)C(C)C]

First, in analogy to the preparation of Intermediate 183, Intermediate 192 was reacted with 4-formylbenzoic acid with borane-pyridine complex to give N-(4-carboxybenzyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide. This compound was then used, in analogy to Intermediate 210, to generate 11 mg (68% of theory) of the title compound.

HPLC (Method 5): Rₜ=1.8 min;

LC-MS (Method 1): Rₜ=1.13 min; MS (ESIpos): m/z=1072 (M+H)⁺.

### Intermediate 217

### N-(5-carboxypentyl)-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

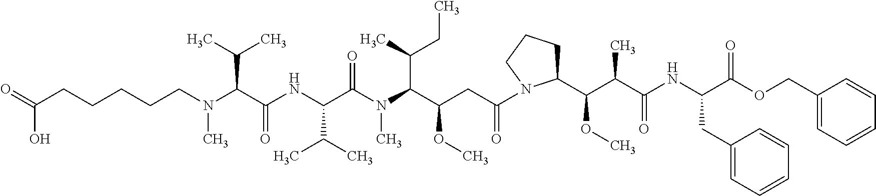 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccccc1)OC)N(C)C(=O)[C@@H](NC(=O)[C@H](C(C)C)N(C)CCCCCC(=O)O)C(C)C]

53 mg (84 μmol) of N-[(9H-fluoren-9-ylmethoxy)carbonyl]-N-methyl-L-valyl-N-[(2R,3S,4S)-1-carboxy-2-methoxy-4-methylhexan-3-yl]-N-methyl-L-valinamide (Intermediate 4) and 45 mg (84 μmol) of benzyl N-{(2R,3R)-3-methoxy-2-methyl-3-[2S)-pyrrolidin-2-yl]propanoyl}-L-phenylalaninate trifluoroacetate (Intermediate 12) were taken up in 2 ml of DMF, 19 μl of N,N-diisopropylethylamine, 14 mg (92 μmol) of HOBt and 17.6 mg (92 μmol) of EDC were added and then the mixture was stirred at RT overnight. Subsequently, the reaction mixture was concentrated and the residue was purified by means of preparative HPLC. This gave 59 mg (68% of theory) of the Fmoc-protected intermediate N-[(9H-fluoren-9-ylmethoxy)carbonyl]-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide.

LC-MS (Method 1): Rₜ=1.55 min; m/z=1044 (M+H)⁺.

57 mg (0.055 mmol) of this intermediate were treated with 1.2 ml of piperidine in 5 ml of DMF to detach the Fmoc protecting group. After concentration and purification by means of preparative HPLC, 39 mg (76% of theory) of the free amine intermediate N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide were obtained as the trifluoroacetate.

HPLC (Method 5): Rₜ=1.9 min;

LC-MS (Method 1): Rₜ=1.01 min; m/z=822 (M+H)⁺.

60 mg (0.06 mmol) of this intermediate were reacted, in analogy to Intermediate 210, with 6-oxohexanoic acid in the presence of borane-pyridine complex. 45 mg (75% of theory) of the title compound were obtained as a foam.

HPLC (Method 5): Rₜ=1.9 min;

LC-MS (Method 1): Rₜ=0.97 min; MS (ESIpos): m/z=9936 (M+H)⁺.

### Intermediate 218

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-1-(benzyloxy)-1-oxo-3-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

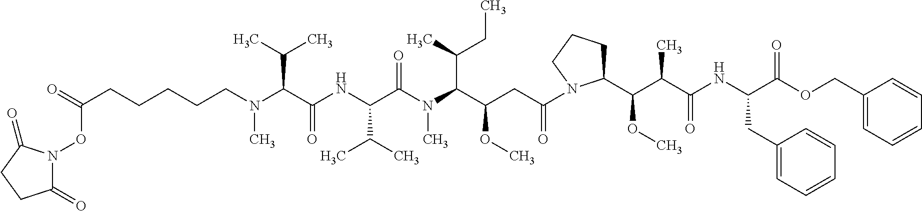 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccccc1)OC)N(C)C(=O)[C@@H](NC(=O)[C@H](C(C)C)N(C)CCCCCC(=O)ON1C(=O)CCC1=O)C(C)C]

This compound was prepared by conversion of 42 mg (0.05 mmol) of Intermediate 217 to the active ester.

Yield: 26 mg (54%)

HPLC (Method 5): Rₜ=2.1 min;

LC-MS (Method 1): Rₜ=1.01 min; MS (ESIpos): m/z=1034 (M+H)⁺.

### Intermediate 219

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide

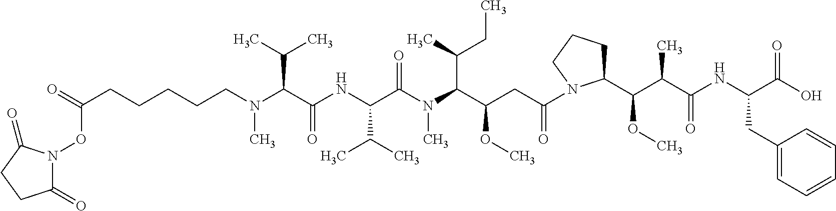 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCCCCC(=O)ON1C(=O)CCC1=O)C(C)C]

20 mg (0.02 mol) of the compound from Intermediate 218 were taken up in 2.4 ml of methanol and hydrogenated over 5% palladium on activated carbon under standard hydrogen pressure at RT for 30 min. The catalyst was then filtered off and the solvent was removed under reduced pressure. The residue was lyophilized from 1:1 acetonitrile/water. This gave 14 mg (92% of theory) of the title compound as a colourless foam.

HPLC (Method 5): Rₜ=1.7 min;

LC-MS (Method 1): Rₜ=0.86 min; MS (ESIpos): m/z=944 (M+H)⁺.

### Intermediate 220

### N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

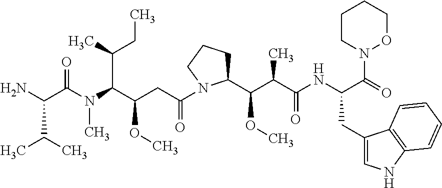 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](N)C(C)C]

500 mg of this intermediate were dissolved in 20 ml of DMF and admixed with 466 mg (3.8 mmol) of Intermediate 191, 382 mg (1.01 mmol) of O-(7-azabenzotriazol-1-yl)-N,N,N′,N′-tetramethyluronium hexafluorophosphate (HATU) and 440 μl (2.5 mmol) of N,N-diisopropylethylamine. The mixture was stirred at RT for 1 h and then concentrated. The residue was taken up in dichloromethane and extracted by shaking first twice with 5% aqueous citric acid solution and then with saturated aqueous sodium hydrogencarbonate solution. The organic phase was concentrated and the residue was purified by flash chromatography on silica gel with 95:5 dichloromethane/methanol as the eluent. The corresponding fractions were combined and the solvent was removed under reduced pressure. After the residue had been dried under high vacuum, 562 mg (65% of theory over both stages) of the Z-protected intermediate were obtained.

562 mg (0.57 mmol) of this intermediate were taken up in 50 ml of methanol and hydrogenated with 155 mg of 10% palladium on activated carbon under standard hydrogen pressure at RT for 20 min. The catalyst was then filtered off and the solvent was removed under reduced pressure. The residue was purified by preparative HPLC. The corresponding fractions were combined, the solvent was evaporated off under reduced pressure and the residue was lyophilized from dioxane. This gave 361 mg (87% of theory) of the title compound as a foam.

HPLC (Method 5): double peak with Rt=1.75 and 1.86 min;

LC-MS (Method 1): double peak at Rt=0.84 min and 0.91 min with the same mass; MS (ESIpos): m/z=944 (M+H)⁺.

### Intermediate 221

### N-{(2S)-2-[(tert-butoxycarbonyl)amino]-3-phenylpropyl}-N-methyl-L-valine

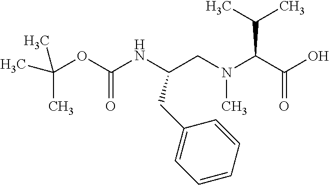 [SMILES: CC(C)[C@@H](C(=O)O)N(C)C[C@H](Cc1ccccc1)NC(=O)OC(C)(C)C]

100 mg (0.76 mmol) of commercially available N-methyl-L-valine and 285 mg (1.14 mmol) of commercially available tert-butyl (2S)-1-oxo-3-phenylpropan-2-yl carbamate were combined in 22 ml of methanol and admixed with 340 mg (3.66 mmol) of borane-pyridine complex and 70 μl of acetic acid. The reaction mixture was stirred at RT overnight. This was followed by concentration under reduced pressure, and the residue was purified by flash chromatography on silica gel with dichloromethane/methanol/17% aqueous ammonia solution as the eluent. After concentration of the corresponding fractions and lyophilization from 1:1 dioxane/water, 259 mg (93% of theory) of the title compound were obtained.

HPLC (Method 12): Rₜ=1.6 min;

LC-MS (Method 11): Rₜ=0.76 min; MS (ESIpos): m/z=365 (M+H)⁺.

### Intermediate 222

### N-[(2S)-2-amino-3-phenylpropyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

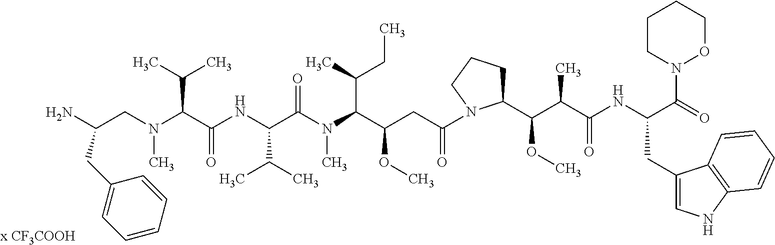 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)C[C@@H](N)Cc1ccccc1)C(C)C]

40 mg (0.11 mmol) of N-{(2S)-2-[(tert-butoxycarbonyl)amino]-3-phenylpropyl}-N-methyl-L-valine (Intermediate 221) were dissolved in 5 ml of DMF and admixed with 80 mg (0.11 mmol) of N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 220), 50 mg (0.13 mmol) of O-(7-azabenzotriazol-1-yl)-N,N,N′,N′-tetramethyluronium hexafluorophosphate (HATU) and 57 μl (2.5 mmol) of N,N-diisopropylethylamine. The mixture was stirred at RT for 1 h and then concentrated. The residue was taken up in ethyl acetate and washed first with 5% aqueous citric acid solution and then with water. The organic phase was concentrated and the residue was purified by means of preparative HPLC. The corresponding fractions were combined and the solvent was removed under reduced pressure. After lyophilization from dioxane, 60 mg (50% of theory) of the protected intermediate were obtained.

HPLC (Method 12): Rₜ=2.2 min;

LC-MS (Method 1): Rₜ=1.17 min; MS (ESIpos): m/z=1073 (M+H)⁺.

60 mg (0.05 mmol) of this intermediate were taken up in 10 ml of dichloromethane, 2 ml of trifluoroacetic acid were added, and the reaction mixture was stirred at RT for 1.5 h. Subsequently, the reaction mixture was concentrated under reduced pressure and the remaining residue was purified by means of preparative HPLC. The corresponding fractions were combined, the solvent was removed under reduced pressure and the residue was lyophilized from dioxane/water. In this way, 25 mg (42% of theory) of the title compound were obtained as a foam.

HPLC (Method 12): Rₜ=1.9 min;

LC-MS (Method 1): Rₜ=0.95 min; MS (ESIpos): m/z=974 (M+H)⁺.

### Intermediate 223

### N-[(2S)-2-({[2-(2,5-dioxo-2,5-dihydro-1H-pyrrol-1-yl)ethyl]carbamoyl}amino)-3-phenylpropyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

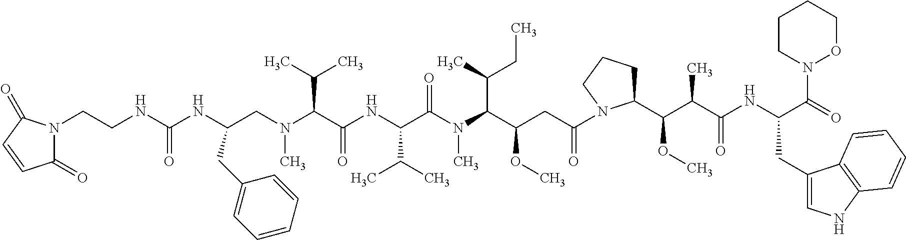 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)C[C@H](Cc1ccccc1)NC(=O)NCCN1C(=O)C=CC1=O)C(C)C]

The preparation was effected in analogy to Intermediate 134, proceeding from 5 mg (4.6 μmol) of Intermediate 222. 3.4 mg (65% of theory) of the title compound were obtained.

HPLC (Method 12): Rₜ=2.0 min;

LC-MS (Method 1): Rₜ=0.99 min; MS (ESIpos): m/z=1140 (M+H)⁺.

### Intermediate 224

### N-[(2S)-2-({[2-(2,5-dioxo-2,5-dihydro-1H-pyrrol-1-yl)ethyl]carbamoyl}amino)propyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

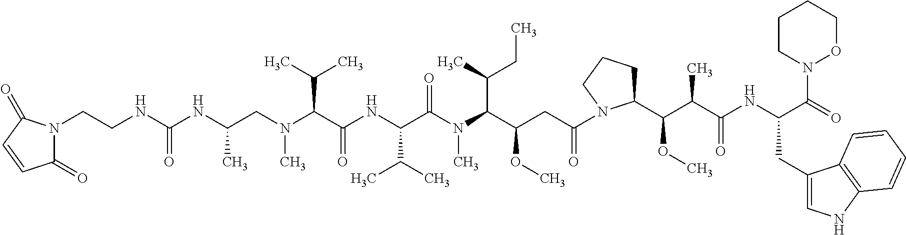 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)C[C@H](C)NC(=O)NCCN1C(=O)C=CC1=O)C(C)C]

The preparation was effected in analogy to the synthesis of Intermediate 223.

HPLC (Method 12): Rₜ=1.9 min;

LC-MS (Method 1): Rₜ=0.92 min; MS (ESIpos): m/z=1064 (M+H)⁺.

### Intermediate 225

### N-(2-aminoethyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

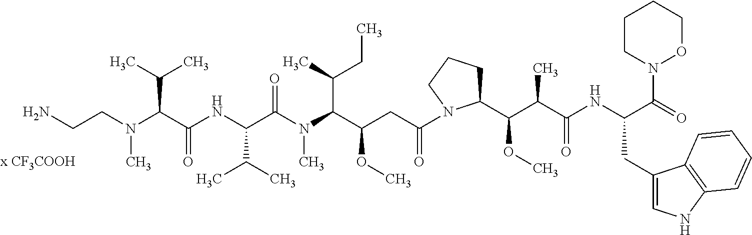 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCN)C(C)C]

100 mg (0.76 mmol) of commercially available N-methyl-L-valine and 182 mg (1.14 mmol) of commercially available tert-butyl 2-oxoethyl carbamate were combined in 20 ml of methanol and admixed with 340 mg (3.66 mmol) of borane-pyridine complex and 65 μl of acetic acid. The reaction mixture was stirred at RT overnight. This was followed by concentration under reduced pressure, and the residue was purified by flash chromatography on silica gel with dichloromethane/methanol/17% aqueous ammonia solution (1540.5) as the eluent. After concentration of the corresponding fractions and lyophilization from 1:1 dioxane/water, 190 mg in 39% purity (35% of theory) of the intermediate were obtained, which were converted further without further purification.

50 mg (0.07 mmol) of this intermediate were dissolved in 10 ml of DMF and admixed with 52 mg (0.07 mmol) of N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide (Intermediate 220), 32 mg (0.09 mmol) of O-(7-azabenzotriazol-1-yl)-N,N,N′,N′-tetramethyluronium hexafluorophosphate (HATU) and 37 μl (0.2 mmol) of N,N-diisopropylethylamine. The mixture was stirred at RT overnight and then concentrated. The residue was taken up in ethyl acetate and extracted by shaking first with 5% aqueous citric acid solution and then with water. The organic phase was concentrated and the residue was purified by means of preparative HPLC. The corresponding fractions were combined and the solvent was removed under reduced pressure. After lyophilization from dioxane, 53 mg (76% of theory) of the protected intermediate were obtained.

HPLC (Method 12): Rₜ=2.0 min;

LC-MS (Method 1): Rₜ=1.02 min; MS (ESIpos): m/z=984 (M+H)⁺.

53 mg (0.05 mmol) of this intermediate were taken up in 10 ml of dichloromethane, 2 ml of trifluoroacetic acid were added, and the reaction mixture was stirred at RT for 30 min Subsequently, the reaction mixture was concentrated under reduced pressure and the remaining residue was purified by means of preparative HPLC. The corresponding fractions were combined, the solvent was removed under reduced pressure and the residue was lyophilized from dioxane/water. In this way, 21 mg (40% of theory) of the title compound were obtained in 65% purity.

HPLC (Method 12): Rₜ=1.7 min;

LC-MS (Method 1): Rₜ=0.87 min; MS (ESIpos): m/z=884 (M+H)⁺.

### Intermediate 226

### N-[2-({[2-(2,5-dioxo-2,5-dihydro-1H-pyrrol-1-yl)ethyl]carbamoyl}amino)ethyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

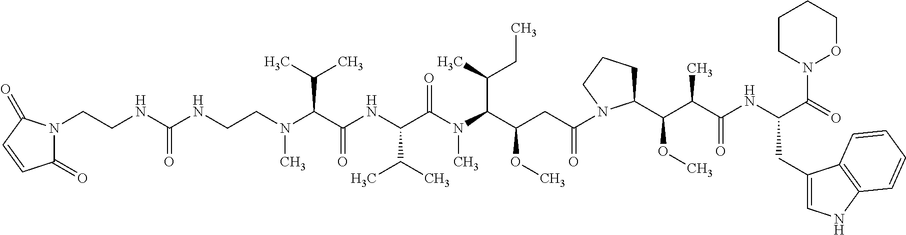 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCNC(=O)NCCN1C(=O)C=CC1=O)C(C)C]

The preparation was effected proceeding from Intermediate 225, in analogy to the synthesis of Intermediate 134. 11.6 mg (59% of theory) of the title compound were obtained.

HPLC (Method 12): Rₜ=1.9 min;

LC-MS (Method 1): Rₜ=0.90 min; MS (ESIpos): m/z=1050 (M+H)⁺.

### Intermediate 227

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-1-(benzyloxy)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

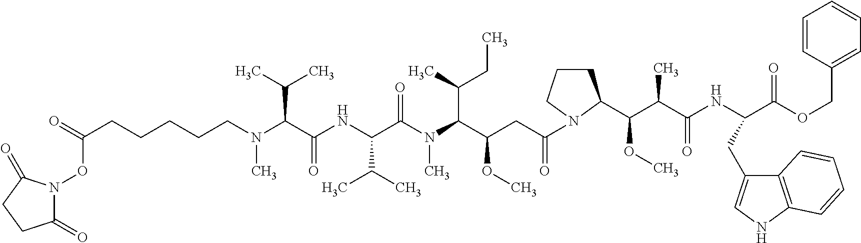 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)OCc1ccccc1)OC)N(C)C(=O)[C@@H](NC(=O)[C@H](C(C)C)N(C)CCCCCC(=O)ON1C(=O)CCC1=O)C(C)C]

This compound was prepared analogously to Intermediate 218, by conversion to the active ester.

Yield: 18 mg (51% of theory)

HPLC (Method 5): Rₜ=2.1 min;

LC-MS (Method 1): Rₜ=0.98 min; MS (ESIpos): m/z=1073 (M+H)⁺.

### Intermediate 228

### (2R,3S)-3-[(tert-butoxycarbonyl)amino]-4-{2-[6-(2,5-dioxo-2,5-dihydro-1H-pyrrol-1-yl)hexanoyl]hydrazino}-4-oxobutan-2-yl(3R,4S,7S,10S)-4-[(2S)-butan-2-yl]-7,10-diisopropyl-3-(2-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-2-oxoethyl)-5,11-dimethyl-6,9-dioxo-2-oxa-5,8,11-triazapentadecan-15-oate

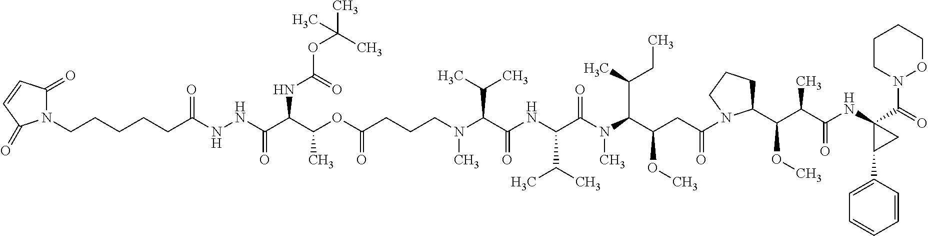 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)O[C@H](C)[C@H](NC(=O)OC(C)(C)C)C(=O)NNC(=O)CCCCCN1C(=O)C=CC1=O)C(C)C]

The title compound was obtained as intermediate in the synthesis of Intermediate 154.

HPLC (Method 12): Rₜ=2.1 min;

LC-MS (Method 1): Rₜ=0.97 min; MS (ESIpos): m/z=1308 (M+H)⁺.

### Intermediate 229

### (2R,3S)-3-acetamido-4-{2-[6-(2,5-dioxo-2,5-dihydro-1H-pyrrol-1-yl)hexanoyl]hydrazino}-4-oxobutan-2-yl (3R,4S,7S,10S)-4-[(2S)-butan-2-yl]-7,10-diisopropyl-3-(2-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-2-oxoethyl)-5,11-dimethyl-6,9-dioxo-2-oxa-5,8,11-triazapentadecan-15-oate

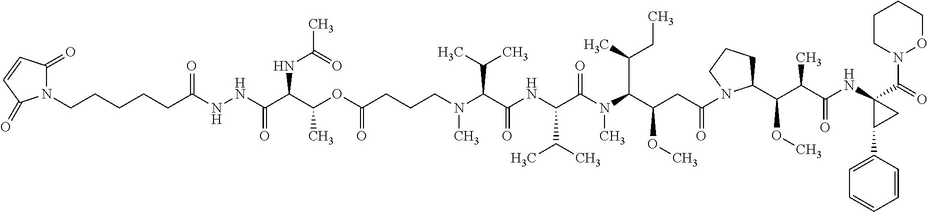 [SMILES: CC[C@H](C)[C@@H]([C@@H](CC(=O)N1CCC[C@H]1[C@H](OC)[C@@H](C)C(=O)N[C@@]1(C(=O)N2CCCCO2)C[C@@H]1c1ccccc1)OC)N(C)C(=O)[C@@H](NC(=O)[C@H](C(C)C)N(C)CCCC(=O)O[C@H](C)[C@H](NC(C)=O)C(=O)NNC(=O)CCCCCN1C(=O)C=CC1=O)C(C)C]

The title compound was prepared from 7.5 mg (2.5 μmol) of Intermediate 154 by acetylation with 2.3 μl of acetic anhydride in 1 ml of DMF in the presence of 0.4 μl of N,N-diisopropylethylamine.

Yield: 1.4 mg (40% of theory)

HPLC (Method 12): Rₜ=1.9 min;

LC-MS (Method 1): Rₜ=0.86 min; MS (ESIpos): m/z=1250 (M+H)⁺.

### Intermediate 230

### (2R,3S)-3-[(tert-butoxycarbonyl)amino]-4-{2-[6-(2,5-dioxo-2,5-dihydro-1H-pyrrol-1-yl)hexanoyl]hydrazino}-4-oxobutan-2-yl (3R,4S,7S,10S)-4-[(2S)-butan-2-yl]-3-(2-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-2-oxoethyl)-7,10-diisopropyl-5,11-dimethyl-6,9-dioxo-2-oxa-5,8,11-triazapentadecan-15-oate

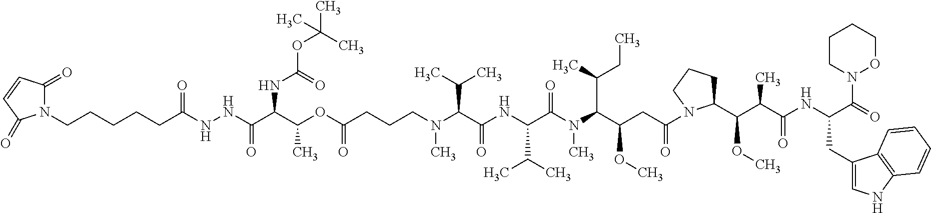 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCC(=O)O[C@H](C)[C@H](NC(=O)OC(C)(C)C)C(=O)NNC(=O)CCCCCN1C(=O)C=CC1=O)C(C)C]

This compound was prepared in analogy to Intermediate 229, proceeding from Intermediate 193. 16 mg (30% of theory over 3 stages) of the title compound were obtained.

HPLC (Method 12): Rₜ=2.0 min;

LC-MS (Method 1): Rₜ=1.02 min; MS (ESIpos): m/z=1335 (M+H)⁺.

### Intermediate 231

### (2R,3S)-3-acetamido-4-{2-[6-(2,5-dioxo-2,5-dihydro-1H-pyrrol-1-yl)hexanoyl]hydrazino}-4-oxobutan-2-yl (3R,4S,7S,10S)-4-[(2S)-butan-2-yl]-3-(2-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-2-oxoethyl)-7,10-diisopropyl-5,11-dimethyl-6,9-dioxo-2-oxa-5,8,11-triazapentadecan-15-oate

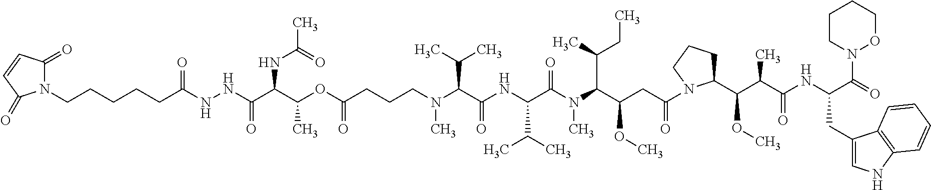 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCC(=O)O[C@H](C)[C@H](NC(C)=O)C(=O)NNC(=O)CCCCCN1C(=O)C=CC1=O)C(C)C]

This compound was prepared from 8 mg (6 μmol) of Intermediate 230, first by deprotection with trifluoroacetic acid and subsequent acetylation with acetic anhydride in DMF in the presence of N,N-diisopropylethylamine. 2 mg (37% of theory over 2 stages) of the title compound were obtained.

HPLC (Method 12): Rₜ=1.9 min;

LC-MS (Method 1): Rₜ=0.88 min; MS (ESIpos): m/z=1277 (M+H)⁺.

### Intermediate 232

### benzyl N-[(4-nitrophenoxy)carbonyl]-beta-alaninate

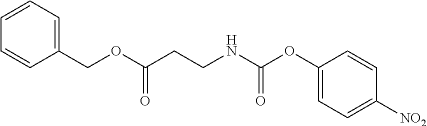 [SMILES: O=C(CCNC(=O)Oc1ccc([N+](=O)[O-])cc1)OCc1ccccc1]

200 mg (0.57 mmol) of commercially available 4-methylbenzenesulphonic acid-benzyl beta-alaninate and 229 mg (1.14 mmol) of 4-nitrophenyl chlorocarbonate were taken up in 15 ml of tetrahydrofuran and the reaction mixture was then heated to reflux for 30 min Subsequently, the reaction mixture was concentrated under reduced pressure and the residue was purified by means of preparative HPLC. After concentration of the corresponding fractions and drying of the residue under high vacuum, 86 mg (44% of theory) of the title compound were obtained.

HPLC (Method 12): Rₜ=1.8 min;

LC-MS (Method 1): Rₜ=1.07 min; MS (ESIpos): m/z=345 (M+H)⁺.

### Intermediate 233

### N-{2-[({3-[(2,5-dioxopyrrolidin-1-yl)oxy]-3-oxopropyl}carbamoyl)amino]ethyl}-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

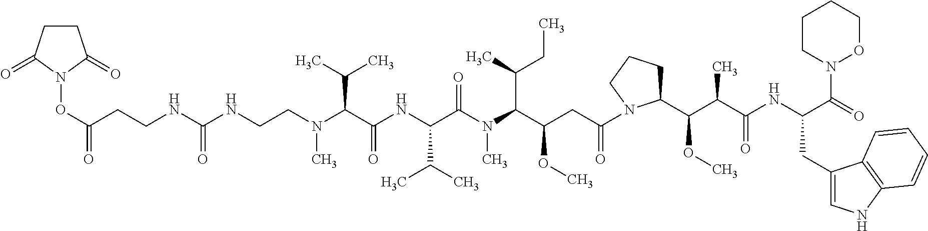 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCNC(=O)NCCC(=O)ON1C(=O)CCC1=O)C(C)C]

13 mg (10 μmol) of Intermediate 225 and 6.7 mg (20 μmol) of Intermediate 232 were dissolved in 3 ml of DMF, and then 7 μl of N,N-diisopropylethylamine were added. The mixture was stirred at RT overnight and then concentrated under high vacuum. The remaining residue was purified by means of preparative HPLC. After concentration of the corresponding fractions and drying of the residue under high vacuum, 5.4 mg (38% of theory) of the protected intermediate were obtained.

HPLC (Method 5): Rₜ=2.1 min;

LC-MS (Method 1): Rₜ=0.6 in; MS (ESIpos): m/z=1089 (M+H)⁺.

5.4 mg (5 μmol) of this intermediate were dissolved in 5 ml of methanol and, after addition of 2 mg of 10% palladium on activated carbon, hydrogenated under standard hydrogen pressure at RT for 20 min. The catalyst was then filtered off and the solvent was removed under reduced pressure. After the residue had been dried under high vacuum, 5 mg (quant.) of the acid intermediate were obtained.

HPLC (Method 12): Rₜ=1.8 min;

LC-MS (Method 1): Rₜ=0.84 min; MS (ESIpos): m/z=999 (M+H)⁺.

5 mg (10 μmol) of this intermediate were dissolved in 1 ml of DMF and admixed with 5.8 mg (50 mmol) of 1-hydroxypyrrolidine-2,5-dione and then with 2.6 μl of N,N-diisopropylethylamine and 3.8 mg (10 μmol) of HATU. After stirring at RT for 20 h, the reaction mixture was concentrated under reduced pressure. The remaining residue was purified by means of preparative HPLC. After lyophilization from 1:1 dioxane/water, 1.1 mg (20% of theory) of the title compound were obtained.

HPLC (Method 12): Rₜ=1.9 min;

LC-MS (Method 1): Rₜ=0.87 min; MS (ESIpos): m/z=1096 (M+H)⁺.

### Intermediate 234

### N-(6-{[(benzyloxy)carbonyl]amino}hexyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

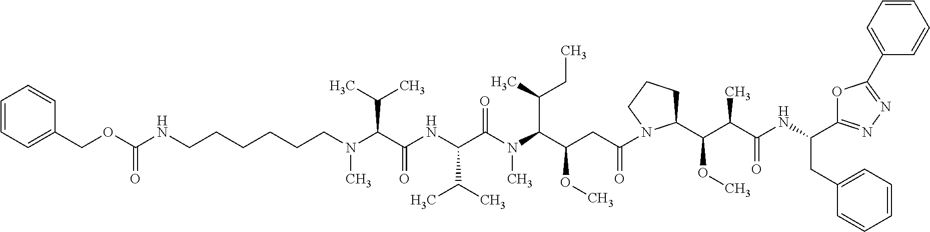 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCCCCNC(=O)OCc1ccccc1)C(C)C]

25 mg (30 μmol) of N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide (Intermediate 55) and 45 mg (180 μmol) of benzyl 6-oxohexyl carbamate were taken up in 3 ml of methanol and acidified with acetic acid. At room temperature, 15 μl (144 μmol; 9.4M) of borane-pyridine complex were subsequently added. The mixture was subsequently stirred at RT for 24 h, and acetic acid and 15 μl (144 μmol; 9.4M) of borane-pyridine complex were added again after 8 h. The reaction mixture was subsequently adjusted to pH 2 with TFA and purified by means of preparative HPLC. The product fractions were combined and concentrated, and the residue was dried under high vacuum. This gave 15 mg (46% of theory) of the title compound as a foam.

LC-MS (Method 1): Rₜ=1.03 min; m/z=1066 (M+H)⁺.

### Intermediate 235

### N-(6-aminohexyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

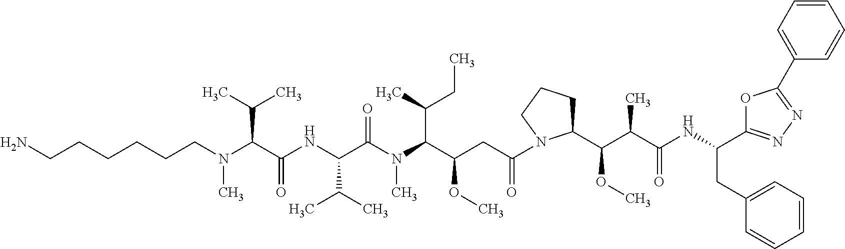 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCCCCN)C(C)C]

15 mg (14 μmol) of N-(6-{[(benzyloxy)carbonyl]amino}hexyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide (Intermediate 234) were taken up in 3 ml of methanol, and 1.8 mg of palladium on charcoal (5%) were added. The reaction mixture was subsequently hydrogenated under standard hydrogen pressure at RT for 2 h. The catalyst was then filtered off and the solvent was removed under reduced pressure. The residue was lyophilized from 1:1 acetonitrile/water. 11 mg (86% of theory) of the title compound were obtained as a foam.

LC-MS (Method 1): Rₜ=0.81 min; m/z=932 (M+H)⁺.

### Intermediate 236

### N-[6-(2,5-dioxo-2,5-dihydro-1H-pyrrol-1-yl)hexyl]-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

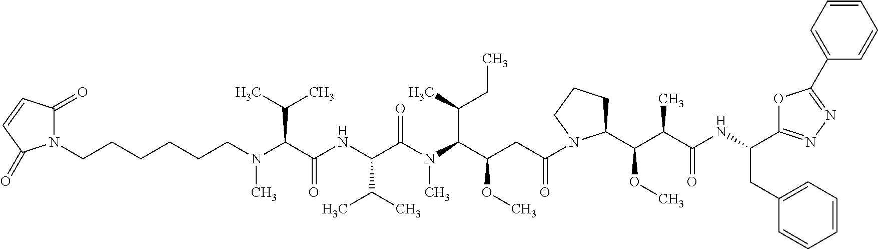 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCCCCN1C(=O)C=CC1=O)C(C)C]

11 mg (12 μmol) of N-(6-aminohexyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide (Intermediate 235) were taken up in 500 μl of 1:1 dioxane/water and admixed with 253 μl of 1M aqueous sodium hydrogencarbonate solution and then with 2.8 mg (18 μmol) of methyl 2,5-dioxo-2,5-dihydro-1H-pyrrole-1-carboxylate. The reaction mixture was stirred at RT for 30 min and then acidified with trifluoroacetic acid. The reaction mixture was purified by means of preparative HPLC. After lyophilization, 0.8 mg (7% of theory) of the title compound was obtained.

LC-MS (Method 1): Rₜ=1.01 min; m/z=1012 (M+H)⁺.

### Intermediate 237

### N-(5-carboxypentyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

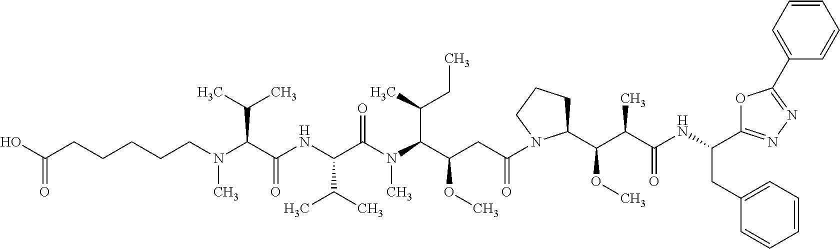 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCCCC(=O)O)C(C)C]

25 mg (30 μmol) of N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide (Intermediate 55) and 23 mg (180 μmol) of 6-oxohexanoic acid were taken up in 3 ml of methanol and acidified with acetic acid. At room temperature, 15 μl (144 μmol; 9.4M) of borane-pyridine complex were subsequently added. The reaction mixture was subsequently stirred at RT for 20 h, and acetic acid and 15 μl (144 μmol; 9.4M) of borane-pyridine complex were added again after 8 h. The reaction mixture was subsequently adjusted to pH 2 with trifluoroacetic acid and purified by means of preparative HPLC. The product fractions were combined and concentrated, and the residue was lyophilized. 21 mg (74% of theory) of the title compound were thus obtained as a foam.

LC-MS (Method 1): Rₜ=0.91 min; m/z=947 (M+H)⁺.

### Intermediate 238

### N-{6-[(2,5-dioxopyrrolidin-1-yl)oxy]-6-oxohexyl}-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

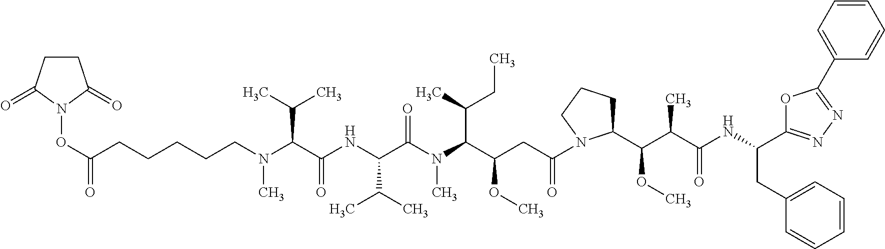 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCCCCC(=O)ON1C(=O)CCC1=O)C(C)C]

21 mg (22 μmol) of Intermediate 237 were dissolved in 1 ml of DMF and admixed with 38 mg (333 μmol) of 1-hydroxypyrrolidine-2,5-dione and then with 2.4 mg (10 μmol) of O-(7-azabenzotriazol-1-yl)-N,N,N′,N′-tetramethyluronium hexafluorophosphate (HATU) and 19 μl of N,N-diisopropylethylamine. After stirring at RT for 2 h, the reaction mixture was purified by means of preparative HPLC. After lyophilization from dioxane, 22 mg (96% of theory) of the title compound were obtained.

LC-MS (Method 1): Rₜ=0.95 min; m/z=1044 (M+H)⁺.

### Intermediate 239

### N-methyl-L-threonyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

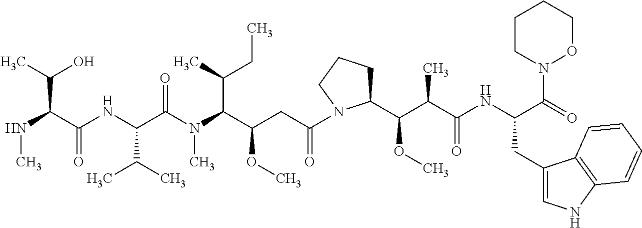 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@@H](NC)C(C)O)C(C)C]

First, N-[(benzyloxy)carbonyl]-N-methyl-L-threonine was released from 237 mg (0.887 mmol) of its dicyclohexylamine salt by taking it up in ethyl acetate and extractive shaking with 5% aqueous sulphuric acid. The organic phase was dried over magnesium sulphate, filtered and concentrated. 14.7 mg (0.055 mmol) of N-[(benzyloxy)carbonyl]-N-methyl-L-threonine were taken up in 3 ml of DMF and admixed successively with 40 mg (0.055 mmol) of Intermediate 220, 12.7 mg (0.066 mmol) of 1-(3-dimethylaminopropyl)-3-ethylcarbodiimide hydrochloride and 10 mg (0.066 mmol) of 1-hydroxy-1H-benzotriazole hydrate. The mixture was subsequently stirred at RT for 2 h. The solvent was then removed under reduced pressure and the residue purified by preparative HPLC. 29 mg (54% of theory) of the Z-protected intermediate were thus obtained.

LC-MS (Method 1): Rₜ=1.15 min; MS (ESIpos): m/z=976 (M+H)⁺.

29 mg (0.003 mmol) of this intermediate were dissolved in 5 ml of methanol and hydrogenated over 5 mg of 5% palladium/charcoal at RT and standard pressure for 1 h. The catalyst was subsequently filtered off and the solvent was evaporated off. The remaining residue was purified by preparative HPLC. 17 mg (54% of theory) of the title compound were obtained.

LC-MS (Method 1): Rₜ=0.77 min; MS (ESIpos): m/z=842 (M+H)⁺.

### Intermediate 240

### N-{6-[(2,5-dioxopyrrolidin-1-yl)oxy]-6-oxohexyl}-N-methyl-L-threonyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

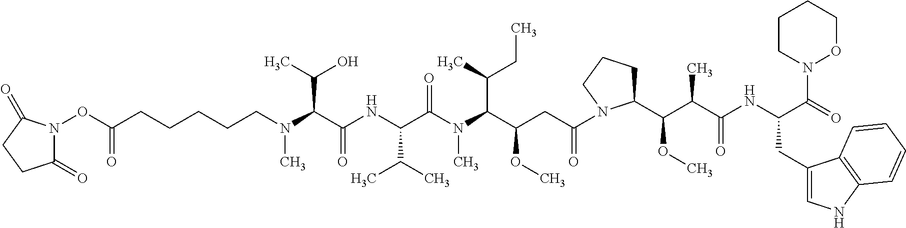 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)O)N(C)CCCCCC(=O)ON1C(=O)CCC1=O)C(C)C]

This compound was prepared in analogy to Intermediate 210 from 15.6 mg (0.016 mmol) of Intermediate 239. 10.8 mg (67% of theory over 2 stages) of the title compound were obtained.

HPLC (Method 5): Rₜ=1.7 min;

LC-MS (Method 1): Rₜ=0.85 min; MS (ESIpos): m/z=1053 (M+H)⁺.

### Intermediate 241

### N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

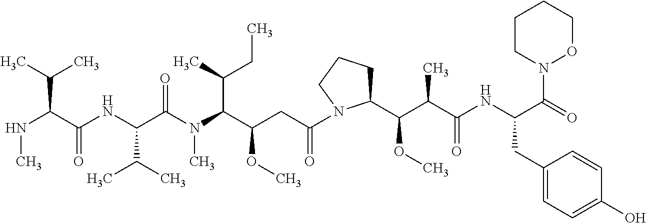 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@@H](NC)C(C)C)C(C)C]

First, in analogy to Intermediate 5, trifluoroacetic acid-(2S)-2-amino-3-(4-hydroxyphenyl)-1-(1,2-oxazinan-2-yl)propan-1-one (1:1) was prepared. This reagent was then used, in analogy to the synthesis described in Intermediate 75, by coupling with N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) in the presence of O-(7-azabenzotriazol-1-yl)-N,N,N′,N′-tetramethyluronium hexafluorophosphate and subsequent detachment of the Boc protecting group by means of trifluoroacetic acid, to prepare the title compound.

HPLC (Method 12): Rₜ=1.7 min;

LC-MS (Method 1): Rₜ=0.85 min; MS (ESIpos): m/z=817 (M+H)⁺.

### Intermediate 242

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

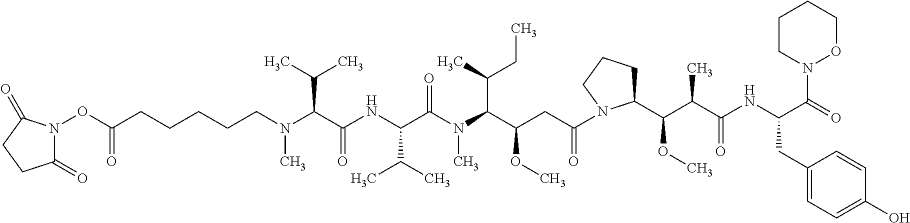 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@H](C(C)C)N(C)CCCCCC(=O)ON1C(=O)CCC1=O)C(C)C]

50 mg (0.05 mmol) of Intermediate 241 were reacted, in analogy to Intermediate 210, with 6-oxohexanoic acid in the presence of borane-pyridine complex. Subsequently, 22.5 mg (0.02 mmol) of the resulting acid were converted to the activated ester. 13.5 mg (36% of theory over 2 stages) of the title compound were obtained.

HPLC (Method 12): Rₜ=1.8 min;

LC-MS (Method 1): Rₜ=0.86 min; MS (ESIpos): m/z=1028 (M+H)⁺.

Alternatively, the title compound can also be obtained by one-hour catalytic hydrogenation of Intermediate 250 at room temperature in methanol over 10% palladium on active carbon under hydrogen standard pressure.

### Intermediate 243

### N-(6-aminohexyl)-N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

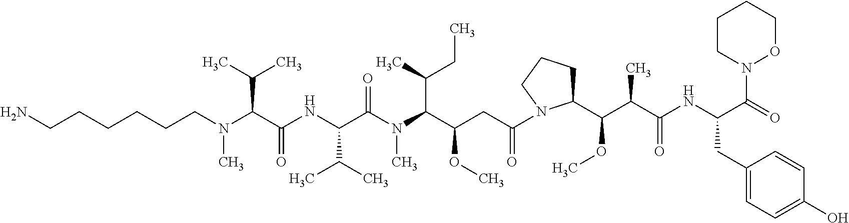 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@H](C(C)C)N(C)CCCCCCN)C(C)C]

The preparation was effected in analogy to Intermediate 78, by reductive alkylation of Intermediate 241 with benzyl 6-oxohexyl carbamate and borane-pyridine complex and subsequent hydrogenation in methanol as the solvent.

Yield: 17.5 mg (34% of theory over 2 stages)

HPLC (Method 12): Rₜ=1.7 min;

LC-MS (Method 1): Rₜ=0.63 min; MS (ESIpos): m/z=916 (M+H)⁺.

### Intermediate 244

### N-[6-(2,5-dioxo-2,5-dihydro-1H-pyrrol-1-yl)hexyl]-N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

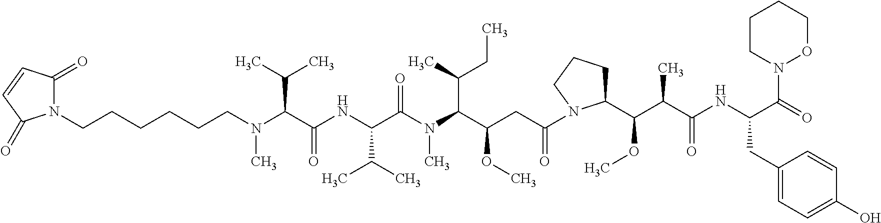 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@H](C(C)C)N(C)CCCCCCN1C(=O)C=CC1=O)C(C)C]

The preparation was effected in analogy to Intermediate 166, proceeding from Intermediate 243.

Yield: 1.3 mg (12% of theory)

HPLC (Method 12): Rₜ=1.9 min;

LC-MS (Method 1): Rₜ=0.89 min; MS (ESIpos): m/z=996 (M+H)⁺.

### Intermediate 245

### 2,5-dioxopyrrolidin-1-yl O-[(3R,4S,7S,10S)-4-[(2S)-butan-2-yl]-3-(2-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-2-oxoethyl)-7,10-diisopropyl-5,11-dimethyl-6,9,15-trioxo-2-oxa-5,8,11-triazapentadecan-15-yl]-N-(tert-butoxycarbonyl)-L-threonyl-beta-alaninate

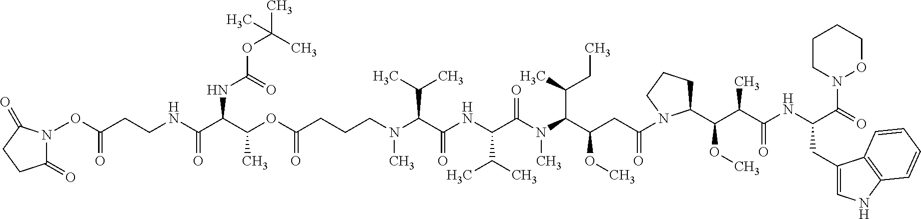 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCC(=O)O[C@H](C)[C@H](NC(=O)OC(C)(C)C)C(=O)NCCC(=O)ON1C(=O)CCC1=O)C(C)C]

First, Intermediate 193, as described under Intermediate 154, was reacted with benzyl N-(tert-butoxycarbonyl)-L-threoninate and then the benzyl ester was removed by hydrogenolysis. 30 mg (0.027 mmol) of the N-[4-({(1S,2R)-1-[(tert-butoxycarbonyl)amino]-1-carboxypropan-2-yl}oxy)-4-oxobutyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide thus obtained were then coupled with 4-methylbenzenesulphonic acid-benzyl beta-alaninate (1:1) in the presence of HATU and the benzyl ester was removed again by hydrogenolysis (yield: 24 mg (71% of theory over 2 stages)). Finally, 10 mg (0.008 mmol) of the resulting acid were converted to the activated ester. After HPLC purification, 2.7 mg (23% of theory) of the title compound were obtained.

HPLC (Method 5): Rₜ=1.9 min;

LC-MS (Method 1): Rₜ=1.01 min; MS (ESIpos): m/z=1295 (M+H)⁺

### Intermediate 246a

### (2S)-2-amino-1-(4-hydroxy-1,2-oxazolidin-2-yl)-3-(1H-indol-3-yl)propan-1-one trifluoroacetic acid (1:1) Diastereomer 1

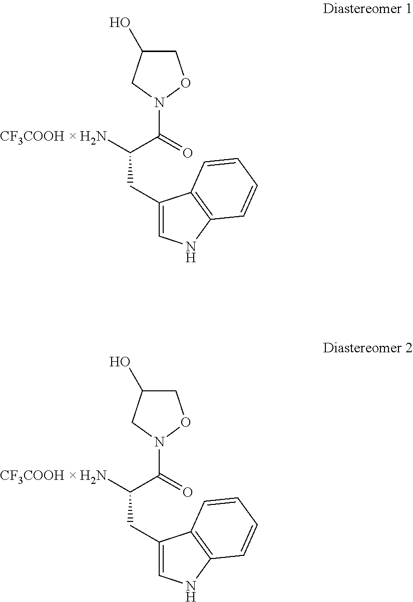

1.6 g (3.982 mmol) of 2,5-dioxopyrrolidin-1-yl N-(tert-butoxycarbonyl)-L-tryptophanate were dissolved in 15 ml of DMF and admixed with 500 mg (3.982 mmol) of 1,2-oxazolidin-4-ol and 100 μl of N,N-diisopropylethylamine. The reaction mixture was stirred at RT overnight. Then another 100 μl of N,N-diisopropylethylamine were added, and the mixture was first treated in an ultrasound bath for 5 h, then stirred at RT overnight, and subsequently concentrated under reduced pressure. The remaining residue was taken up in ethyl acetate and shaken first twice with 5% citric acid solution, then with saturated sodium hydrogencarbonate solution and finally with water. The organic phase was concentrated and the residue was separated into the diastereomers by flash chromatography on silica gel with 95:5 dichloromethane/methanol as the eluent. The corresponding fractions of both diastereomers were combined and the solvent was removed under reduced pressure. After the residues had been dried under high vacuum, 272 mg (18% of theory) of Diastereomer 1 (R_{f}=0.18 (95:5 dichloromethane/methanol) and 236 mg (16% of theory) of Diastereomer 2 (R_{f}=0.13 (95:5 dichloromethane/methanol), and also 333 mg (22% of theory) of a mixed fraction of the Boc-protected intermediates were obtained.

Under standard conditions, 5 ml of trifluoroacetic acid in 20 ml of dichloromethane were used to detach the Boc protecting group from 272 mg (725 μmol) of Diastereomer 1 of this intermediate and, after lyophilization from dioxane/water, 290 mg (quant) of the title compound were obtained in 75% purity and used in the next stage without further purification.

HPLC (Method 12): Rₜ=1.1 min;

LC-MS (Method 13): Rₜ=1.80 min; MS (ESIpos): m/z=276 (M+H)⁺

### Intermediate 246b

### (2S)-2-amino-1-(4-hydroxy-1,2-oxazolidin-2-yl)-3-(1H-indol-3-yl)propan-1-one trifluoroacetic acid (1:1) Diastereomer 2

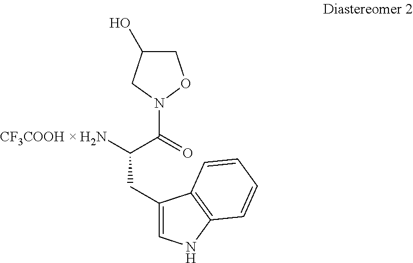

Under standard conditions, 5 ml of trifluoroacetic acid in 20 ml of dichloromethane were used to detach the Boc protecting group from 236 mg (630 μmol) of Diastereomer 2 of the intermediate described in 246a and, after concentration, stirring with diethyl ether and drying of the residue under high vacuum, 214 mg (76%) of the title compound were obtained.

LC-MS (Method 13): Rₜ=1.84 min; MS (ESIpos): m/z=276 (M+H)⁺

### Intermediate 247a

### N-{6-[(2,5-dioxopyrrolidin-1-yl)oxy]-6-oxohexyl}-N-methyl-L-valyl-N-[(3R,4S,5S)-1-{(2S)-2-[(1R,2R)-3-{[(2S)-1-(4-hydroxy-1,2-oxazolidin-2-yl)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

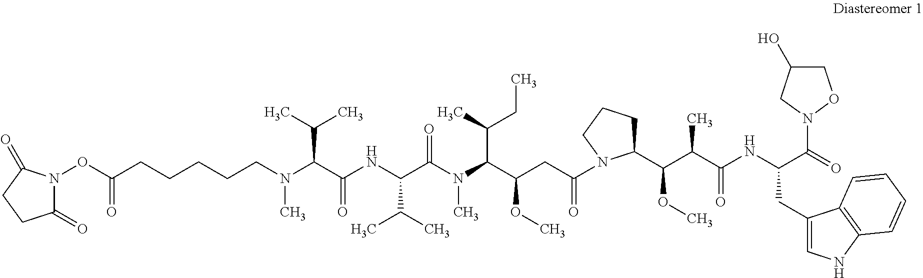 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CC(O)CO1)OC)N(C)C(=O)[C@@H](NC(=O)[C@H](C(C)C)N(C)CCCCCC(=O)ON1C(=O)CCC1=O)C(C)C]

To synthesize this compound, the coupling of Intermediates 26 and 246a with subsequent detachment of the Boc protecting group was first performed as described for Intermediate 74. Subsequently, the alkylation with 6-oxohexanoic acid in the presence of borane-pyridine complex and subsequent conversion of the acid to the active ester were performed as described in Intermediate 210. The title compound was purified by preparative HPLC.

HPLC (Method 12): Rₜ=1.8 min;

LC-MS (Method 1): Rₜ=0.86 min; MS (ESIpos): m/z=1053 (M+H)⁺

### Intermediate 247b

### N-{6-[2,5-dioxopyrrolidin-1-yl)oxy]-6-oxohexyl}-N-methyl-L-valyl-N-[(3R,4S,5S)-1-{(2S)-2-[(1R,2R)-3-{[(2S)-1-(4-hydroxy-1,2-oxazolidin-2-yl)-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

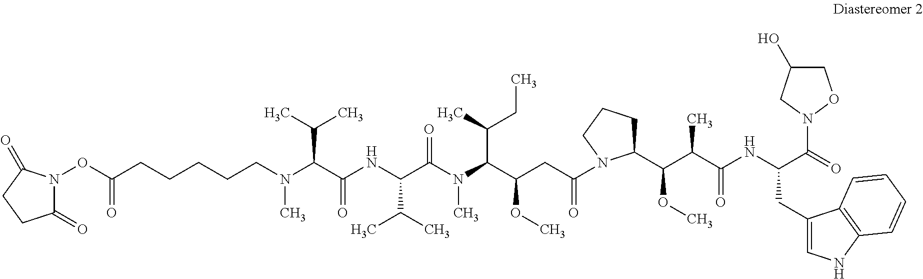 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CC(O)CO1)OC)N(C)C(=O)[C@@H](NC(=O)[C@H](C(C)C)N(C)CCCCCC(=O)ON1C(=O)CCC1=O)C(C)C]

To synthesize this compound, the coupling of Intermediates 26 and 246b with subsequent detachment of the Boc protecting group was first performed as described for Intermediate 74. Subsequently, the alkylation with 6-oxohexanoic acid in the presence of borane-pyridine complex and subsequent conversion of the acid to the active ester were performed as described in Intermediate 210. The title compound was purified by preparative HPLC.

HPLC (Method 12): Rₜ=1.8 min;

LC-MS (Method 1): Rₜ=0.86 min; MS (ESIpos): m/z=1053 (M+H)⁺

### Intermediate 248

### N-(5-carboxypentyl)-N-methyl-L-valyl-N-[(3R,4S,5S)-1-{(2S)-2-[(1R,2R)-3-{[(2S)-1-tert-butoxy-3-(4-hydroxyphenyl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

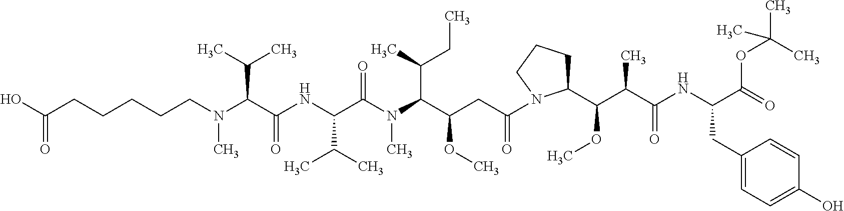 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)OC(C)(C)C)OC)N(C)C(=O)[C@@H](NC(=O)[C@H](C(C)C)N(C)CCCCCC(=O)O)C(C)C]

First, in analogy to the synthesis described in Intermediate 86, by coupling N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) and tert-butyl L-tyrosinate in the presence of O-(7-azabenzotriazol-1-yl)-N,N,N′,N′-tetramethyluronium hexafluorophosphate and subsequent detachment of the Boc protecting group by means of trifluoroacetic acid to obtain the tert-butyl ester (stirring with trifluoroacetic acid in dichloromethane for 40 min), the amine compound tert-butyl N-[(2R,3R)-3-methoxy-3-{(2S)-1-[(3R,4S,5S)-3-methoxy-5-methyl-4-(methyl{(2S)-3-methyl-2-[(N-methyl-L-valyl)amino]butyl}amino)heptanoyl]pyrrolidin-2-yl}-2-methylpropanoyl]-L-tyrosinate was prepared as the trifluoroacetate. 38 mg (0.04 mmol) of this compound were then used, in analogy to the preparation of Intermediate 210, by reaction with 4-oxohexanoic acid in the presence of borane-pyridine complex, to obtain 31 mg (99% of theory) of the title compound.

HPLC (Method 12): Rₜ=1.8 min;

LC-MS (Method 1): Rₜ=0.88 min; MS (ESIpos): m/z=918 (M+H)⁺.

### Intermediate 249

### Trifluoroacetic acid-N-methyl-L-valyl-N-[(3R,4S,5S)-1-{(2S)-2-[(1R,2R)-3-{[(2S)-3-[4-(benzyl-oxy)phenyl]-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide(1:1)

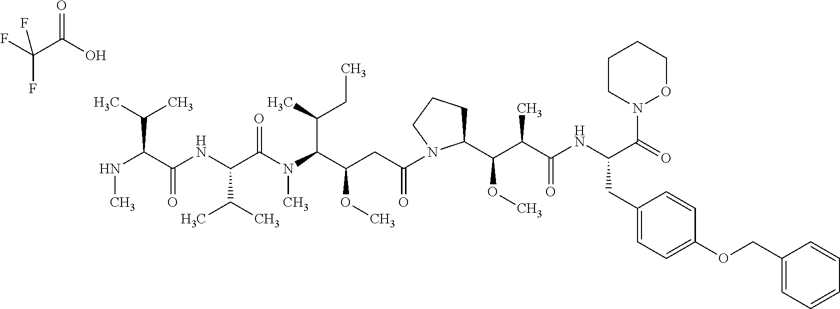 [SMILES: O=C(O)C(F)(F)F ; CC[C@H](C)[C@@H]([C@@H](CC(=O)N1CCC[C@H]1[C@H](OC)[C@@H](C)C(=O)N[C@@H](Cc1ccc(OCc2ccccc2)cc1)C(=O)N1CCCCO1)OC)N(C)C(=O)[C@@H](NC(=O)[C@@H](NC)C(C)C)C(C)C]

First, in analogy to Intermediates 5 and 6, starting from O-benzyl-N-(tert-butoxycarbonyl)-L-tyro sine, trifluoroacetic acid-(2S)-2-amino-3-[4-(benzyloxy)phenyl]-1-(1,2-oxazinan-2-yl)propan-1-one(1:1) was prepared. Then, from this building block, in analogy to the synthesis described in Intermediate 75, coupling with N-(tert-butoxycarbonyl)-N-methyl-L-valyl-N-[(3R,4S,5S)-1-{(2S)-2-[(1R,2R)-2-carboxy-1-methoxypropyl]pyrrolidin-1-yl}-3-methoxy-5-methyl-1-oxoheptan-4-yl]-N-methyl-L-valinamide (Intermediate 26) in the presence of O-(7-azabenzotriazol-1-yl)-N,N,N′,N′-tetramethyluronium hexafluorophosphate and subsequent elimination of the Boc protecting group by means of trifluoroacetic acid gave the title compound.

HPLC (Method 12): Rₜ=2.15 min;

LC-MS (Method 1): Rₜ=0.99 min; MS (ESIpos): m/z=908 (M+H)⁺.

### Intermediate 250

### N-{6-[(2,5-Dioxopyrrolidin-1-yl)oxy]-6-oxohexyl}-N-methyl-L-valyl-N-[(3R,4S,5S)-1-{(2S)-2-[(1R,2R)-3-{[(2S)-3-[4-(benzyloxy)phenyl]-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

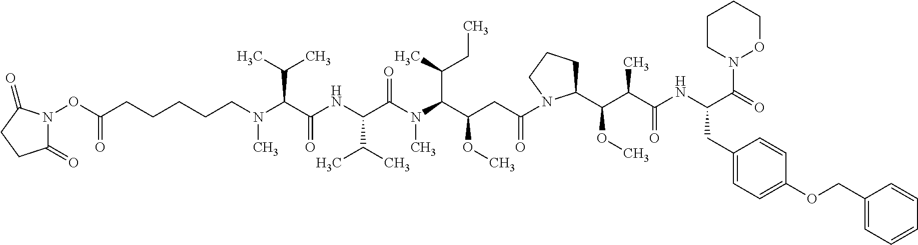 [SMILES: CC[C@H](C)[C@@H]([C@@H](CC(=O)N1CCC[C@H]1[C@H](OC)[C@@H](C)C(=O)N[C@@H](Cc1ccc(OCc2ccccc2)cc1)C(=O)N1CCCCO1)OC)N(C)C(=O)[C@@H](NC(=O)[C@H](C(C)C)N(C)CCCCCC(=O)ON1C(=O)CCC1=O)C(C)C]

100 mg (0.088 mmol) of Intermediate 249 were reacted in analogy to Intermediate 210 with 6-oxanoic acid in the presence of borane-pyridine complex. Then 30 mg (0.029 mmol) of the resulting acid were converted into the activated ester. This gave 15 mg (40% of theory over 2 stages) of the title compound.

HPLC (Method 12): Rₜ=2.26 min;

LC-MS (Method 1): Rₜ=1.05 min; MS (ESIpos): m/z=1119 (M+H)⁺.

### Intermediate 251

### N-[4-(2-{5-[(2,5-Dioxopyrrolidin-1-yl)oxy]-5-oxopentanoyl}-2-methylhydrazino)-4-oxobutyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

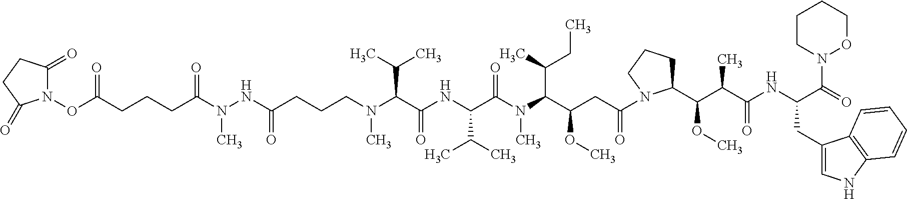 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCC(=O)NN(C)C(=O)CCCC(=O)ON1C(=O)CCC1=O)C(C)C]

First of all, 30 mg (0.032 mmol) of Intermediate 193 were converted into the activated N-hydroxysuccinimide ester. 10.3 mg (0.009 mmol) of this active ester were dissolved in 2 ml of DMF, admixed with 2.7 mg (0.018 mmol) of tert-butyl 1-methylhydrazinecarboxylate and 8 μL N,N-diisopropylethylamine, and stirred at RT for 16 h. This operation was repeated, and then the batch was concentrated and the residue which remained was purified by preparative HPLC. Concentration of the corresponding fractions and drying under a high vacuum gave 5.4 mg (43%) of the intermediate.

LC-MS (Method 1): Rₜ=0.99 min; MS (ESIpos): m/z=1054 (M+H)⁺.

The Boc protecting group was removed from 3.5 mg (0.002 mmol) of this intermediate, using trifluoroacetic acid in dichloromethane. Following concentration and drying under a high vacuum, the residue was taken up in 4 ml of dichloromethane and admixed with 1,1′-[(1,5-dioxopentane-1,5-diyl)bis(oxy)]dipyrrolidine-2,5-dione and 2 μl of N,N-diisopropylethylamine. After stirring at RT for 1 h, the batch was concentrated and the residue which remained was purified by preparative HPLC. Concentration of the corresponding fractions and drying under a high vacuum gave 1.4 mg (44%) of the title compound.

HPLC (Method 5): Rₜ=1.6 min;

LC-MS (Method 1): Rₜ=0.88 min; MS (ESIpos): m/z=1166 (M+H)⁺.

### Intermediate 252

### N-(2-{2-[2-(2-Carboxyethoxy)ethoxy]ethoxy}ethyl)-N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

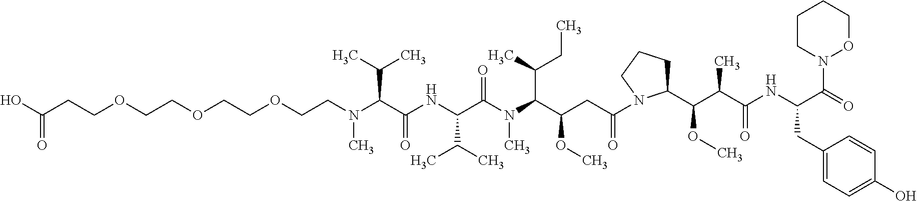 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@H](C(C)C)N(C)CCOCCOCCOCCC(=O)O)C(C)C]

100 mg (0.088 mmol) of Intermediate 249 and 109 mg (0.350 mmol) of Intermediate 167 were combined in 10 ml of methanol and admixed with 39 mg (0.42 mmol) of borane-pyridine complex and 15 μl of acetic acid. The batch was stirred at RT overnight. Then the same amounts of borane-pyridine complex and of acetic acid were added once again, and the batch was stirred for a further 24 h at RT. It was then concentrated under reduced pressure, and the residue was purified by means of preparative HPLC. Concentration of the corresponding fractions and lyophilization from dioxane/water 1:1 gave 98 mg (93% of theory) of the bis-benzyl intermediate. This intermediate was taken up in 18.5 ml of methanol and subjected to catalytic hydrogenation over 5% palladium on activated carbon under standard hydrogen pressure at room temperature for 1 h. Filtration, concentration, and lyophilization of the residue from dioxane gave 73 mg (87% of theory) of the title compound.

HPLC (Method 12): Rₜ=1.85 min;

LC-MS (Method 1): Rₜ=0.84 min; MS (ESIpos): m/z=1021 (M+H)⁺.

### Intermediate 253

### N-{2-[2-(2-{3[(2,5-Dioxopyrrolidin-1-yl)oxy]-3-oxopropoxy}ethoxy)ethoxy]ethyl}-N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

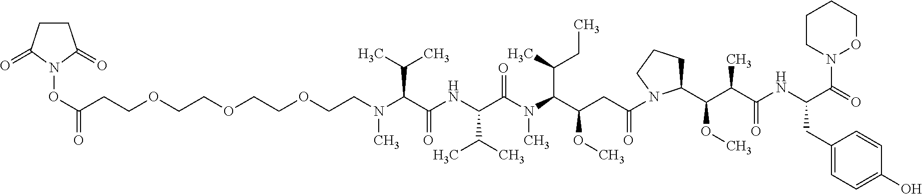 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@H](C(C)C)N(C)CCOCCOCCOCCC(=O)ON1C(=O)CCC1=O)C(C)C]

22 mg (22 μmol) of Intermediate 252 were dissolved in 8.5 ml of DMF and admixed with 25 mg (215 μmol) of 1-hydroxypyrrolidine-2,5-dione and subsequently with 12.3 mg (32 μmol) of O-(7-azabenzotriazol-1-yl)-N,N,N′,N′-tetramethyluronium hexafluorophosphate (HATU) and 37 μl of N,N-diisopropylethylamine. After stirring at RT for 2 h, the reaction mixture was purified by means of preparative HPLC. Lyophilization from dioxane gave 16 mg (62% of theory) of the title compound.

HPLC (Method 5): Rₜ=1.57 min;

LC-MS (Method 1): Rₜ=0.8 min; MS (ESIpos): m/z=1118 (M+H)⁺.

### Intermediate 254

### N-(2-{2-[2-(2-Carboxyethoxy)ethoxy]ethoxy}ethyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

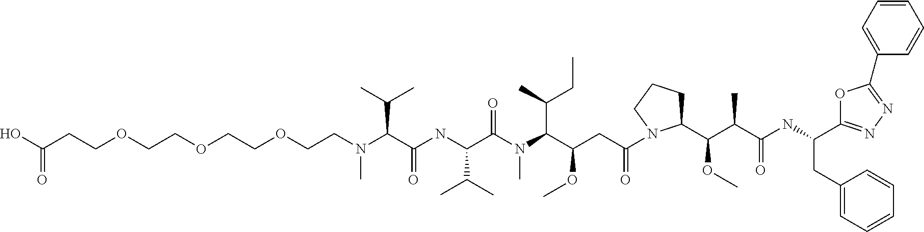 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCOCCOCCOCCC(=O)O)C(C)C]

10 mg (0.012 mmol) of Intermediate 55 and 11 mg (0.036 mmol) of Intermediate 167 were combined in 1 ml of methanol and admixed with 5.4 mg (0.058 mmol) of borane-pyridine complex and 1 μl of acetic acid. The batch was stirred at RT overnight. Then the same amounts of borane-pyridine complex and of acetic acid were added once again, and the batch was stirred at RT for a further 20 h. It was then concentrated under reduced pressure and the residue was purified by means of preparative HPLC. Concentration of the corresponding fractions and lyophilization from dioxane/water 1:1 gave 8 mg (58% of theory) of bis-benzyl intermediate. This intermediate was taken up in 2 ml of methanol and subjected to catalytic hydrogenation over 5% palladium on activated carbon under standard hydrogen pressure at room temperature for 1 h. Filtration, concentration, and lyophilization of the residue from dioxane gave 7 mg (95% of theory) of the title compound.

LC-MS (Method 1): Rₜ=0.99 min; MS (ESIpos): m/z=1036 (M+H)⁺.

### Intermediate 255

### N-{2-[2-(2-{3[(2,5-Dioxopyrrolidin-1-yl)oxy]-3-oxopropoxy}ethoxy)ethoxy]ethyl}-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

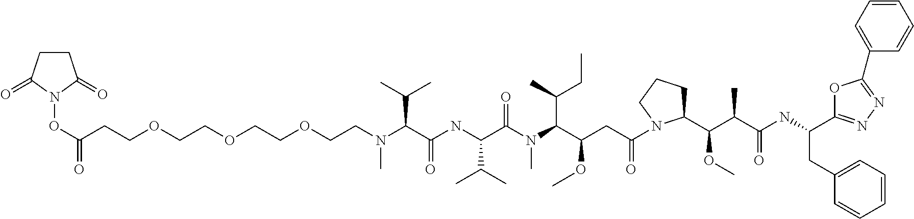 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCOCCOCCOCCC(=O)ON1C(=O)CCC1=O)C(C)C]

7.3 mg (7 μmol) of Intermediate 254 were dissolved in 0.3 ml of DMF and admixed with 12 mg (106 μmol) of 1-hydroxypyrrolidine-2,5-dione and subsequently with 13.5 mg (35 μmol) of O-(7-azabenzotriazol-1-yl)-N,N,N′,N′-tetramethyluronium hexafluorophosphate (HATU) and 6 μl of N,N-diisopropylethylamine. After stirring at RT for 2 h, the reaction mixture was purified by means of preparative HPLC. Lyophilization from dioxane gave 7.7 mg (79% of theory) of the title compound.

LC-MS (Method 1): Rₜ=0.97 min; MS (ESIpos): m/z=1134 (M+H)⁺.

### Intermediate 256

### N-(2-{2-[2-(2-Aminoethoxy)ethoxy]ethoxy}ethyl)-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

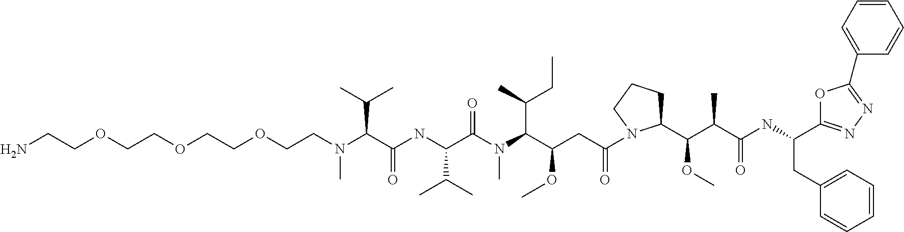 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCOCCOCCOCCN)C(C)C]

20 mg (0.02 mmol) of Intermediate 55 were subjected to reductive amination in analogy to the preparation of Intermediate 254, with benzyl (2-{2-[2-(2-oxoethoxy)ethoxy]ethoxy}ethyl)carbamate (Intermediate 172) in the presence of borane-pyridine complex. The Z protecting group was subsequently removed by hydrogenolysis, using 5% palladium on carbon as catalyst and in methanol as solvent, and 21 mg (85% of theory over 2 stages) of the title compound were prepared.

LC-MS (Method 1): Rₜ=0.85 min; MS (ESIpos): m/z=1008 (M+H)⁺.

### Intermediate 257

### N-[2-(2-{2-[2-(2,5-Dioxo-2,5-dihydro-1H-pyrrol-1-yl)ethoxy]ethoxy}ethoxy)ethyl]-N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(5-phenyl-1,3,4-oxadiazol-2-yl)ethyl]amino}propyl]pyrrolidin-1-yl}-5-methyl-1-oxoheptan-4-yl]-N-methyl-L-valinamide

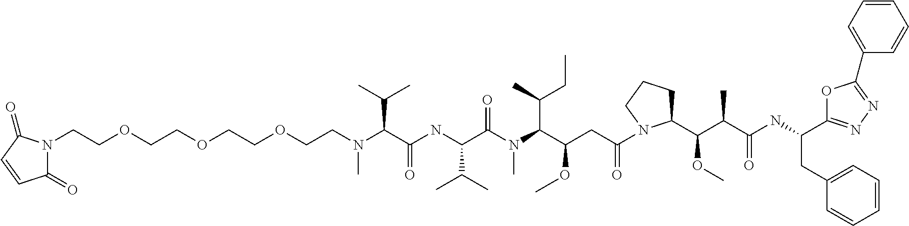 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H](C(C)C)N(C)CCOCCOCCOCCN1C(=O)C=CC1=O)C(C)C]

21 mg (20.8 μmol) of Intermediate 256 were taken up in 1 ml of dioxane/water 1:1 and then admixed with 4.9 mg (31.2 μmol) of methyl 2,5-dioxo-2,5-dihydro-1H-pyrrole-1-carboxylate and also with 42 μl of 1M aqueous sodium hydrogen carbonate solution. The reaction mixture was stirred at RT for 30 min. Then a further 374 μl of the 1M aqueous sodium hydrogen carbonate solution were added, and the reaction mixture was stirred at RT for a further 30 min and then concentrated under reduced pressure. The residue which remained was purified by means of preparative HPLC. Lyophilization gave 4.5 mg (20% of theory) of the title compound, as a colourless foam.

LC-MS (Method 1): Rₜ=1.04 min; MS (ESIpos): m/z=1088 (M+H)⁺.

### Intermediate 258

### N-{2-[2-(2-{3-[(2,5-Dioxopyrrolidin-1-yl)oxy]-3-oxopropoxy}ethoxy)ethoxy]ethyl}-N-methyl-L-valyl-N-[(3R,4S,5S)-1-{(2S)-2-[(1R,2R)-3-{[(2S)-3-[4-(benzyloxy)phenyl]-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

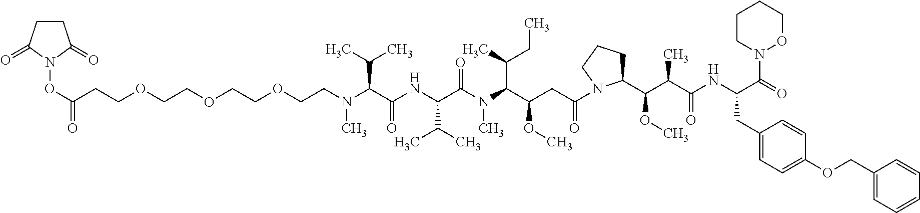 [SMILES: CC[C@H](C)[C@@H]([C@@H](CC(=O)N1CCC[C@H]1[C@H](OC)[C@@H](C)C(=O)N[C@@H](Cc1ccc(OCc2ccccc2)cc1)C(=O)N1CCCCO1)OC)N(C)C(=O)[C@@H](NC(=O)[C@H](C(C)C)N(C)CCOCCOCCOCCC(=O)ON1C(=O)CCC1=O)C(C)C]

This compound was prepared starting from Intermediate 249 by reductive alkylation using tert-butyl 3-{2-[2-(2-oxoethoxy)ethoxy]ethoxy}propanoate, followed by t-butyl ester cleavage and conversion into the N-hydroxysuccinimide ester.

HPLC (Method 5): Rₜ=1.96 min;

LC-MS (Method 1): Rₜ=1.11 min; MS (ESIpos): m/z=1208 (M+H)⁺.

### Intermediate 259

### N-(5-Carboxypentyl)-N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide

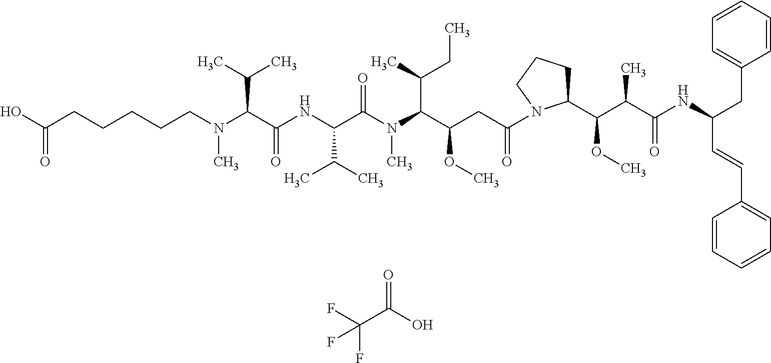 [SMILES: CC[C@H](C)[C@@H]([C@@H](CC(=O)N1CCC[C@H]1[C@H](OC)[C@@H](C)C(=O)N[C@H](/C=C/c1ccccc1)Cc1ccccc1)OC)N(C)C(=O)[C@@H](NC(=O)[C@H](C(C)C)N(C)CCCCCC(=O)O)C(C)C ; O=C(O)C(F)(F)F]

9.6 mg (8.4 μmol) of N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate (Intermediate 58) and 6.5 mg (51 μmol) of 6-oxohexanoic acid were taken up in 769 μl of methanol and acidified with acetic acid. Then, at room temperature, 4 μl (40 μmol) of borane-pyridine complex were added. The reaction mixture was subsequently stirred at RT for 20 h. The reaction mixture was then adjusted to a pH of 2 with trifluoroacetic acid, and purified by means of preparative HPLC. The production fractions were combined and concentrated and the residue was lyophilized. This gave 8 mg (93% of theory) of the title compound, as a foam.

LC-MS (Method 1): Rₜ=1.06 min; m/z=1019 (M+H)⁺.

### Intermediate 260

### N-{6-[(2,5-Dioxopyrrolidin-1-yl)oxy]-6-oxohexyl}-N-methyl-L-valyl-N-[(3R,4S,5S)-1-{(2S)-2-[(1R,2R)-3-{[(2S,3E)-1,4-diphenylbut-3-en-2-yl]amino}-1-methoxy-2-methyl-3-oxopropyl]pyrrolidin-1-yl}-3-methoxy-5-methyl-1-oxoheptan-4-yl]-N-methyl-L-valinamide

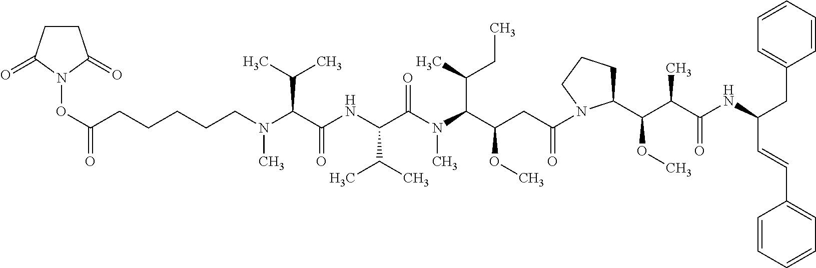 [SMILES: CC[C@H](C)[C@@H]([C@@H](CC(=O)N1CCC[C@H]1[C@H](OC)[C@@H](C)C(=O)N[C@H](/C=C/c1ccccc1)Cc1ccccc1)OC)N(C)C(=O)[C@@H](NC(=O)[C@H](C(C)C)N(C)CCCCCC(=O)ON1C(=O)CCC1=O)C(C)C]

7.5 mg (7.4 μmol) of Intermediate 259 were dissolved in 332 μl of DMF and admixed with 12.7 mg (110 μmol) of 1-hydroxypyrrolidine-2,5-dione and subsequently with 14 mg (37 μmol) of O-(7-azabenzotriazol-1-yl)-N,N,N′,N′-tetramethyluronium hexafluorophosphate (HATU) and 6 μl of N,N-diisopropylethylamine. After stirring at RT for 2 h, the reaction mixture was purified by means of preparative HPLC. Lyophilization from dioxane gave 4 mg (55% of theory) of the title compound.

LC-MS (Method 1): Rₜ=1.19 min; m/z=1002 (M+H)⁺.

### Intermediate 261

### N-{2-[2-(2-{3-[(2,5-Dioxopyrrolidin-1-yl)oxy]-3-oxopropoxy}ethoxy)ethoxy]ethyl}-N-methyl-L-valyl-N-[(3R,4S,5S)-3-methoxy-1-{(2S)-2-[(1R,2R)-1-methoxy-2-methyl-3-{[(1S,2R)-1-(1,2-oxazinan-2-ylcarbonyl)-2-phenylcyclopropyl]amino}-3-oxopropyl]pyrrolidin-1-yl}-5-methyl-1-oxoheptan-4-yl]-N-methyl-L-valinamide

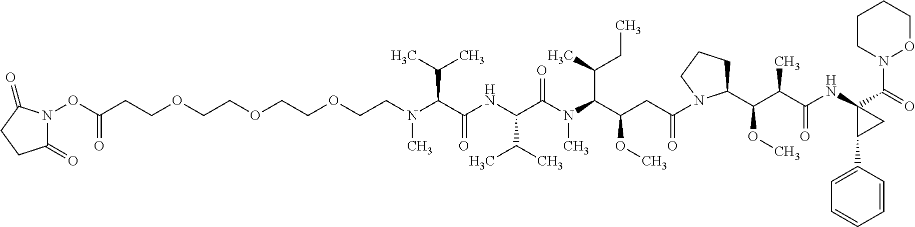

This compound was prepared starting from Intermediate 16 by reductive alkylation with benzyl 3-{2-[2-(2-oxoethoxy)ethoxy]ethoxy}propanoate, subsequent benzyl ester cleavage by hydrogenolysis, and conversion into the N-hydroxysuccinimide ester.

HPLC (Method 5): Rₜ=1.83 min;

LC-MS (Method 1): Rₜ=0.93 min; MS (ESIpos): m/z=1114 (M+H)⁺.

### B: PREPARATION OF ANTIBODY-DRUG CONJUGATES (ADCS)

### B-1. Generation of Anti-FGFR2 Antibodies

The human anti-FGFR2 antibodies M048-D01-hIgG1 and M048-D01-hIgG1-b of the present invention were isolated by phage display technology, using the naïve Fab antibody library n-CoDeR from Bioinvent International AB (Lund, Sweden; described in Söderling et al., Nat. Biotech. 2000, 18:853-856). One screening hit was the parental Fab fragment M048-D01. The variable region of the heavy chain Vh of this Fab fragment is given by SEQ ID NO: 21; the variable region of the light chain Vl is given by SEQ ID NO: 22. Following identification of the M048-D01 Fab fragment, it was expressed under the name M048-D01-hIgG1-b (SEQ ID NO: 10 for the heavy chain, and SEQ ID NO: 9 for the light chain) in the form of human IgG. For efficient cloning, the first three amino acids of the N-terminus of the heavy chain of M048-D01-hIgG1-b [EVQ] were also expressed, alternatively, as QVE. This variation gave the antibody M048-D01-hIgG1 (SEQ ID NO: 8 for the heavy chain and SEQ ID NO: 7 for the light chain). The variable regions Vh and Vl of M048-D01-hIgG1 are given by SEQ ID NO: 11 and SEQ ID NO: 12; the variable regions Vh and Vl of M048-D01-hIgG1-b are given by SEQ ID NO: 13 and SEQ ID NO: 14. Both antibodies have the same CDR sequences, which are given by SEQ ID NOs: 15 (H-CDR1), 16 (H-CDR2), 17 (H-CDR3), 18 (L-CDR1), 19 (L-CDR2) and 20 (L-CDR3).

The antibodies GAL-FR21-mIgG1 and GAL-FR22-mIgG2a are described in WO2010/054265 as mIgG1 (GAL-FR21) and mIgG2b (GAL-FR22). GAL-FR21 is defined therein via Vh (SEQ ID NO: 4 in WO2010054265) and via Vl (SEQ ID NO: 1 in WO2010054265), and GAL-22 is defined via Vh (SEQ ID NO: 8 in WO2010054265) and via Vl (SEQ ID NO: 7 in WO2010054265). For the studies described in the present application, Vl and Vh of GAL-FR21 were reformatted into the murine IgG1 format, resulting in the antibody GAL-FR21-mIgG1 (SEQ ID NO: 3 and SEQ ID NO: 4 of the present application). Vl and Vh of GAL-FR22 were reformatted into the murine IgG2a format, resulting in the antibody GAL-FR22-mIgG2a (SEQ ID NO: 5 and SEQ ID NO: 6 of the present application).

### B-2. Expression of Anti-FGFR2 Antibodies

The antibodies M048-D01-hIgG1, M048-D01-hIgG1-b, GAL-FR21-mIgG1 and GAL-FR22-mIgG2a were produced transiently in mammalian cell culture.

The expression constructs were produced as described below:

In order to transfer the Vh and Vl regions of the M048-D01 Fab originating from the phage display into the IgG format, and in order to change the expression system from E. coli to mammalian cells, the Vh and Vl sequences from the phage display E. coli clone were amplified using PCR primers. The flanking restriction enzyme cleavage sites were introduced at the 5′ and 3′ ends both of Vh and of Vl. These restriction enzyme cleavage sites were used for the cloning of Vh and Vl into an expression vector which contained the IgG backbone.

The E. coli cells were incubated in a test tube with 100 μl of water at 95° C. for 10 minutes and then placed on ice for 5 minutes. Following brief vortexing, the solution was clarified by centrifugation. The supernatant separated off was used for DNA amplification. The PCR reactions were carried out separately for Vh and Vl. This was done using specific primer pairs with BamHI and NotI cleavage sites for Vl and MfeI, and MfeI and BlpI cleavage sites for Vh. The PCR reactions were carried out using the AccuPrime Pfx polymerase (Invitrogen, #12344-024) according to manufacturer instructions. The PCR products were analysed on 1% agarose gels. To obtain compatible ends, the expression vectors and PCR products were digested according to manufacturer instructions with the corresponding restriction endonucleases at 37° C. for 2 hours (see Table 1). Digestion was halted by incubation at 70° C. for 15 minutes. The resulting fragments were ligated into the expression vector, and E. coli or mammalian cells were transformed with the constructs by standard methods.

The DNA sequences of the Vh and Vl domains or of complete light chains of certain antibodies (GAL-FR21-mIgG1, GAL-FR22-mIgG2a and M048-D01-hIgG1-b) were synthesized by Geneart gene synthesis and gene optimizer technology for mammalian gene expression (life technologies, Grand Island, N.Y., USA). During the gene synthesis, the V domains on sequences coding for a mammalian signal peptide were fused with upstream Kozaki sequence. Flanking restriction cleavage sites were inserted on 5′ and 3′ ends of the synthesized DNA constructs. These restriction cleavage sites were used for the cloning of the Vh and Vl or the light chains into an expression vector which contained the coding constant regions of the IgG.

The expression vectors and the Geneart constructs were digested according to manufacturer instructions with the respective restriction endonucleases at 37° C. for 2 hours, to give compatible ends (see Table 1). Digestion was halted by incubation at 70° C. for 15 minutes. The resulting fragments were ligated, and E. coli cells were transformed with the ligates. Plasmids obtained from these transformants were used to transform mammalian cells according to standard methods.

TABLE 1
Restriction cleavage sites for the cloning of Vh and
Vl or of the light chain into IgG expression vectors

Restriction cleavage site
for the cloning of:

| Antibody | VH | VL or light chain* |
|---|---|---|
| GAL-FR21-mIgG1 | HindIII-BlpI | XbaI-BsiWI |
| GAL-FR22-mIgG2a | HindIII-BlpI | XbaI-BsiWI |
| M048-D01-hIgG1# | MfeI-BlpI | BamHI-NotI* |
| M048-D01-hIgG1-b | HindIII-BlpI | XbaI-NotI* |

*Cloning of the complete light chain
#Cloning of the PCR product into EcoRI-BlpI and BamHI-NotI-digested expression vectors.

The antibodies were expressed transiently in mammalian cell culture, as described in Tom et al. (Tom et al., Chapter 12 in Methods Express: Expression Systems, edited by Michael R. Dyson and Yves Durocher, Scion Publishing Ltd, 2007): for the expression of the anti-FGFR2 antibodies, for example M048-D01-hIgG1, GAL-FR21-mIgG1 and GAL-FR22-mIgG2a, HEK293 6E cells were transfected transiently with a suitable CMV promoter-based expression plasmid. The cell culture scale was either up to 1.5 1 in a shake flask or 10 1 in a “wave-bag”. Expression took place at 37° C. for 5-6 days in F17 medium (Invitrogen) supplemented with tryptone TN1 (Organotechnie) with 1% “FCS ultra low IgG” (Invitrogen) and 0.5 mM valproic acid.

Alternatively, the anti-FGFR2 antibodies, for example M048-D01-hIgG1-b, were expressed in a stably transfected Chinese hamster ovary (CHO) cell line. This was done using a one-vector system. Fermentation took place in bioreactors on a different scale in a fed-batch process.

The parental Fab fragment M048-D01 (Vh: SEQ ID NO: 21, Vl: SEQ ID NO: 22) belonging to the M048-D01-based antibodies from the phase display was expressed as follows: 20-50 ml of LB medium (admixed with 0.1 mg/ml ampicillin and 0.1% glucose) were inoculated with a preliminary culture of a corresponding E. coli clone, which contained the initial pBif vector, which was lacking the geneIII sequence, but which had had the M048-D01 Fab sequence cloned into it. Production of the sFab was started by addition of 0.5 mM IPTG (final concentration). Incubation took place at 30° C. overnight at 250 rpm.

### B-3. Purification of the FGFR-2 Antibodies

The antibodies, for example M048-D01-hIgG1, M048-D01-hIgG1-b, GAL-FR21-mIgG1 and GAL-FR22-mIgG2a, were obtained from the cell culture supernatants. The cell supernatants were clarified by centrifugation from cells. The cell supernatant was subsequently purified by affinity chromatography on a MabSelect Sure (GE Healthcare) chromatography column. For this purpose, the column was equilibrated in DPBS pH 7.4 (Sigma/Aldrich), the cell supernatant was applied and the column was washed with around 10 column volumes of DPBS pH 7.4+500 mM NaCl. The antibodies were eluted in 50 mM Na acetate pH 3.5+500 mM NaCl, and subsequently purified further by gel filtration chromatography on a Superdex 200 column (GE Heathlcare) in DPBS pH 7.4.

The parental M048-D01 Fab fragment expressed in E. coli was purified as follows: the E. coli cells were harvested by centrifugation and lysed by incubation at 4° C. for 1 hour in lysis buffer (20% sucrose (w/v), 30 mM TRIS, 1 mM EDTA, pH 8.0, 1 mg/ml lysozyme (Sigma L-6876) and 2.5 U/ml Benzonase (Sigma E1014)). Thereafter the same volume of PBS was added. After that, the clarified supernatant was applied to Dynabeads for his-tag isolation (Invitrogen, 101-03D) and the mixture was swirled at 4° C. for 2 hours. Thereafter the matrix was washed three times with buffer 1 (50 mM Na phosphate buffer, pH 7.4, 300 mM NaCl, 5 mM imidazole, 0.01% Tween 20). Subsequently a single washing step in buffer 2 was carried out (PBS admixed with 0.005% of Tween 20). Lastly, the Fabs were eluted with buffer E (10 mM Na phosphate buffer, pH 7.4, 300 mM NaCl, 300 mM imidazole) and concentrated using PBS buffer in Vivaspin 500 concentrators (cut-off 10 000, from GE, 28-9322-25).

### B-4. Construction of the Cross-Reactivity Profile of the M048-D01-Based Antibodies

The cross-reactivity of the human antibodies M048-D01-hIgG1 and M048-D01-hIgG1-b of the present application was determined using the parental Fab fragment M048-D01 (comprising Vh: SEQ ID NO: 21 and Vl: SEQ ID NO: 22).

The M048-D01 Fab fragment was tested in an ELISA for binding to the various FGF receptor variants listed in Table 2.

TABLE 2
List of recombinant proteins used for establishing
the cross-reactivity profile of the FGFR2 binders

| | | Cat. No. |
|---|---|---|
| Protein | Origin | (RnD Systems) |
| hFGFR2β-Fc (IIIb) | Human | 665-FR |
| mFGFR2β-Fc (IIIb) | Mouse | 708-MF |
| hFGFR2α-Fc (IIIb) | Human | 663-FR |
| hFGFR2β-Fc (IIIc) | Human | 684-FR |
| hFGFR1β-Fc (IIIc) | Human | 661-FR |
| hFGFR1β-Fc (IIIb) | Human | 765-FR |
| hFGFR3-Fc (IIIc) | Human | 766-FR |
| hFGFR3-Fc (IIIb) | Human | 1264-FR |
| hFGFR4-Fc | Human | 685-MF |
| mFGFR2β-Fc (IIIc) | Mouse | 716-MF |
| mFGFR3-Fc (IIIc) | Mouse | 710-MF |
| hTRAIL-Fc | Human | 630-TR |

All of the variants took the form of Fc fusion proteins in carrier-free preparations. The proteins were biotinylated according to manufacturer instructions, using a 2-fold molar excess of biotin-LC-NHS (Pierce; Cat. No. 21347) and desalted using Zeba desalting columns (Pierce; Cat. No. 89889). For the ELISA, 96-well plates pretreated with streptavidin (Pierce, 15500) were loaded overnight at 4° C. with 1 μg/ml biotinylated protein. Wells which had been loaded with biotinylated TRAIL-Fc served as a reference. The next day the plates were washed 3× with PBST (1×PBS admixed with 0.05% Tween 20 (Sigma, P7949)), treated with blocking buffer (PBST admixed with 3% BSA (Sigma A4503)), and again washed three times with PBST. 100 μl of the purified Fab (1 μg/ml) were added, and incubation was carried out at room temperature for 1 hour. After threefold washing with PBST, an HRP-coupled anti-hIgG (Fab-specific) (diluted 1:2500, Sigma, A0293) was added, and incubation took place at room temperature for 1 hour. The colour reaction was activated by addition of 50 μl of TMB (Invitrogen, 2023) and halted after 5-15 minutes by addition of 50 μl of H₂SO₄ (Merck, 1120801000). The colour reaction was monitored at 450 nm in a plate reader (Tecan). The signal strengths of the wells containing TRAIL-Fc were used as background values, and the signal-to-background ratios were calculated as summarized in Table 3.

TABLE 3
Summary of the ELISA data on the cross-reactivity of M048-D01

| | hFGFR2β- | hFGFR2β- | hFGFR2α- | mFGFR2β- | mFGFR2β- | hFGFR1β- | hFGFR1β- | hFGFR3- | hFGFR3- | mFGFR3- | |
|---|---|---|---|---|---|---|---|---|---|---|---|
| | Fc | Fc | Fc | Fc | Fc | Fc | Fc | Fc | Fc | Fc | hFGFR4- |
| | (IIIb) | (IIIc) | (IIIb) | (IIIb) | (IIIc) | (IIIb) | (IIIc) | (IIIb) | (IIIc) | (IIIc) | Fc |
| M048- | +++ | +++ | +++ | +++ | +++ | 0 | 0 | 0 | 0 | 0 | 0 |
| D01 | | | | | | | | | | | |

Signal-to-background ratios:
0: <2;
+: 2-3;
++: 3-5;
+++: >5

As is evident from Table 2, the M048-D01-based antibodies M048-D01-hIgG1 and M048-D01-hIgG1-b bind to human and murine FGFR2, and do so independently of whether the forms in question are alpha or beta isoforms, or are IIIb and IIIc splice forms. As is likewise apparent from the table, the M048-D01-based antibodies of the invention do not bind to FGFR1, FGFR3 and FGFR4.

### B-5. Epitope Mapping by Means of CLIPS Technology

In order to test the binding characteristics of the M048-D01-based antibodies, an intensive epitope mapping operation was carried out on the basis of Pepscan's proprietary “Chemically Linked peptides on Scaffolds” (CLIPS) Technologie (Timmerman et al., J. Mol. Recognit. 2007, 20:283-99). In all, 8653 different CLIPS peptides were designed, from 15 amino acids to 30 amino acids in length, which cover linear, conformational and discontinuous epitopes on the human FGFR2. The peptides were synthesized on peptide arrays. The human antibody M048-D01-hIgG1 was tested on the peptide arrays in an ELISA-based procedure. The peptides which gave the highest ELISA values were analysed to isolate common, similar amino acid sequences.

In order to reconstruct discontinuous epitopes of the target molecule, a library of structured peptides was synthesized. The CLIPS technology allows peptides to be structured in individual loops, double loops, triple loops, sheet-like loops, helix-like loops, and combinations of these structural elements: for this purpose, CLIPS templates are coupled to cysteine residues of the peptide arrays. For example, a 0.5 mM solution of the T2 CLIPS template 1,3-bis(bromomethyl)benzene was dissolved in ammonium bicarbonate (20 mM, pH 7.9)/acetonitrile (1:1 (v/v)). This solution was added to the peptide arrays. The CLIPS template bound to the side chains of 2 cysteines of the peptides present in the peptide arrays (455 well plate with 3 μl wells). The peptide arrays were cautiously swirled in the solution for 30 to 60 minutes, with complete coverage by the solution. At the conclusion, the arrays were washed thoroughly with water in excess and were treated in disrupt buffer (1% SDS, 0.1% beta-mercaptoethanol in PBS (pH 7.2)) at 70° C. for 30 minutes in an ultrasound bath. The treatment in the ultrasound bath was subsequently repeated in water for a further 45 minutes. T3 CLIPS-bearing peptides were prepared in a similar way.

The binding of the antibodies to each peptide was tested in a PEPSCAN-based ELISA (Sloostra et al., Molecular Diversity 1996, 1: 87-96). The peptide arrays were preincubated (1 h, 20° C.) with 5% to 100% of binding buffer. The binding buffer consisted of 1% Tween 80, 4% horse serum and 5% ovalbumin (w/v) in solution in PBS. After a washing step, the peptide arrays were incubated at 4° C. overnight with primary antibody solution (1 to 5 μg/ml) in 1% Tween 80 in PBS. After a further washing step, the peptide arrays were incubated at 25° C. for an hour in a 1/1000 dilution of an antibody peroxidase conjugate (anti-human-IgG) in 100% binder buffer. After a further washing step, the peroxidase substrate 2,2′-azino-di-3-ethylbenzothiazolinesulphonate (ABTS) and 2 μl/ml 3% strength H₂O₂ were added. After an hour, the development of colour was measured and was quantified using a CCD camera and an image processing system.

The crude data obtained in this method are optical values which range from 0-3000 mAU (milli-absorption-units).

The result is that all the M048-D01-based antibodies bind on an epitope which consists of the 15 N-terminal residues of FGFR2 (¹RPSFSLVEDTTLEPE¹⁵). Analysis of 1257 CLIPS and linear peptides gave consistently high ELISA values for N-terminal peptides.

The N-terminal residues (¹RPSFSLVEDTTLEPE¹⁵) are present in all splice variants of human FGFR2, independently of alternative splicing in domain D3, which results in the IIIb and IIIc isoforms. The epitope is also present if the domain D1 is removed from the full-length FGFR2 (FGFR2 alpha, SEQ ID NO: 1) by splicing, resulting in the shorter beta-form of FGFR2 (SEQ ID NO: 2). In this case the epitope is situated directly before the domain D2.

A matter of particular interest is the fact that the N-terminal sequence is conserved in human, mouse, rat and rhesus monkey. This allows the broad inter-species cross-reactivity of the M048-D01 antibodies. The binding epitope of M048-D01-hIgG1 and M048-D01-hIgG1-b, two example antibodies of the present invention, is marked as the striped box in FIG. 1.

### B-6. General Process for Coupling to Cysteine Side Chains

The antibodies used in the coupling reactions were as follows:

M048-D01-hIgG1
M048-D01-hIgG1-b

Added to a solution of the corresponding antibody in PBS buffer in the concentration range between 1 mg/ml and 10 mg/ml were 3 equivalents of tris(2-carboxyethyl)phosphine hydrochloride (TCEP), in solution in PBS buffer, and the mixture was stirred at RT for 1 hour. Subsequently, depending on the desired loading, between 2 and 10 equivalents of the maleimide precursor compound or halide precursor compound for coupling from the Intermediates 102, 103, 105-109, 111-114, 117-126, 128, 129, 132-146, 148-155, 157, 159-161, 166, 171, 175-177, 184, 189, 194-195, 199-201, 205, 209, 223-224, 226, 228-231, 236, 244 and 257 were added as a solution in DMSO. The amount of DMSO here ought not to exceed 10% of the overall volume. The batch was stirred at RT for 60-120 minutes and then applied to PD 10 columns (Sephadex® G-25, GE Healthcare) equilibrated with PBS, and eluted with PBS buffer. Optionally a concentration procedure was carried out additionally by means of ultracentrifugation.

Normally, unless otherwise indicated, 5 mg of the corresponding antibody in PBS buffer were used for the reduction and the subsequent coupling. Following purification via the PD10 column, this gave, in each case, solutions of the corresponding ADC in 3.5 ml of PBS buffer. The particular protein concentration indicated was then determined for these solutions. Furthermore, the loading of the antibody (drug/mAb ratio) was determined in accordance with the methods described below. This process was used to prepare the immunoconjugates represented in Examples 1, 3, 5-6, 8, 10-12, 14, 15, 27 and 32.

In the structural formulae illustrated, the definition of AK_{1A} and AK_{1B} is as follows

AK_{1A}=anti-FGFR2 antibody M048-D01-hIgG1 (partially reduced)-S§¹
AK_{1B}=anti-FGFR2 antibody M048-D01-hIgG1-b (partially reduced)-S§¹
where
§¹ denotes the link with the succinimide group,
and
S stands for the sulphur atom of a cysteine residue of the partially reduced antibody.

### B-7. General Process for Coupling to Lysine Side Chains

The following antibodies were used in the coupling reactions:

M048-D01-hIgG1
M048-D01-hIgG1-b
GAL-FR21-mIgG1
GAL-FR22-mIgG2a

Added to a solution of the corresponding antibody in PBS buffer in the concentration range between 1 mg/ml and 10 mg/ml were, depending on the desired loading, between 2 and 5 equivalents of the precursor compound for coupling from the Intermediates 104, 110, 115, 116, 127, 130, 131, 147, 156, 158, 162, 169, 178, 185, 190, 202, 206, 210-216, 218, 219, 227, 233, 238, 240, 242, 245, 247a, 247b, 250, 251, 253, 255, 258 and 260-261 as a solution in DMSO. After 30 minutes of stirring at RT, the same amount of precursor compound in DMSO was added again. The amount of DMSO here ought not to exceed 10% of the overall volume. After a further 30 minutes of stirring at RT, the batch was applied to PD 10 columns (Sephadex® G-25, and eluted with PBS buffer. Optionally a concentration procedure was carried out additionally by means of ultrafiltration. If necessary, for more effective removal of low molecular mass constituents, the concentration by ultrafiltration was repeated after re-dilution with PBS buffer.

Normally, unless otherwise indicated, 5 mg of the corresponding antibody in PBS buffer were used for the coupling. Following purification via the PD10 column, this gave, in each case, solutions of the corresponding ADC in 3.5 ml of PBS buffer. The particular protein concentration indicated was then determined for these solutions and the loading of the antibody (drug/mAb ratio) was determined in accordance with the methods described below.

This process was used to prepare the immunoconjugates represented in Examples 2, 4, 7, 9, 13, 16-17, 25, 26, 28-31 and 33-35.

In the structural formulae illustrated, the definition of AK_{2A}, AK_{2B}, AK_{2D} and AK_{2E} is as follows

AK_{2A}=anti-FGFR2 antibody M048-D01-hIgG1-NH§²
AK_{2B}=anti-FGFR2 antibody M048-D01-hIgG1-b-NH§²
AK_{2D}=anti-FGFR2 antibody GAL-FR21-mIgG1-NH§²
AK_{2E}=anti-FGFR2 antibody GAL-FR22-mIgG2a-NH§²
where
§² denotes the link with the carbonyl group
and
NH stands for the side chain amino group of a lysine residue of the antibody.

### B-8. General Process for Preparing Cysteine Adducts

10 μmol of the above-described maleimide precursor compounds were taken up in 3 ml of DMF and admixed with 2.1 mg (20 μmol) of L-cysteine. The reaction mixture was stirred at RT for 2 hours, then concentrated under reduced pressure and subsequently purified by preparative HPLC.

In the structural formulae illustrated, the definition of Cys is as follows

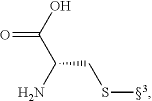

where
§³ denotes the link with the linker-toxophore unit.

### B-9. General Process for Preparing Lysine Adducts

10 μmol of the above-described active ester precursor compounds were taken up in 5 ml of DMF and admixed, in the presence of 30 μmol of N,N-diisopropylethylamine, with α-amino-protected L-lysine. The reaction mixture was stirred at RT for 2 hours, then concentrated under reduced pressure and subsequently purified by preparative HPLC. The protective group was then removed by known methods.

### Further Purification and Characterization of the Conjugates of the Invention

After reaction had taken place, in certain cases the reaction mixture was concentrated, by ultrafiltration, for example, and then desalted and purified by means of chromatography, for example using a Sephadex® G-25. Elution took place with, for example, phosphate-buffered saline (PBS). The solution was subsequently subjected to sterile filtration and freezing. An alternative option is to lyophilize the conjugate.

### B-10. Determination of the Toxophore Loading

The toxophore loading of the resultant solutions of the conjugates described in the working examples, in PBS buffer, was determined as follows:

The toxophore loading of lysine-linked ADCs was determined by mass-spectrometric determination of the molecular weights of the individual conjugate species. In this case, to start with, the antibody conjugates were deglycosylated by means of PNGaseF, and the sample was acidified and, following HPLC separation, was analysed by mass spectrometry using an ESI-MicroTofQ (Bruker Daltonik). All of the spectra were added via the signal in the TIC (Total Ion Chromatogram), and the molecular weight of the various conjugate species was calculated on the basis of MaxEnt Deconvolution. Following signal integration of the different species, the DAR (Drug/Antibody Ratio) was then calculated.

For protein identification, in addition to the molecular weight determination, a tryptic digestion was carried out after deglycosylation and/or denaturing, and this digestion, after denaturing, reduction and derivatization, confirmed the identity of the protein on the basis of the tryptic peptides detected.

The toxophore loading of cysteine-linked conjugates was determined via reversed-phase chromatography of the reduced and denatured ADC. The ADC solution (1 mg/mL, 50 μL) was admixed with guanidinium hydrochloride (GuHCl) (28.6 mg) and with a solution of DL-dithiothreitol (DTT) (500 mM, 3 μL). The mixture was incubated at 55° C. for an hour and analysed by HPLC.

The HPLC analysis was carried out on an Agilent 1260 HPLC System with detection at 220 nm. The column used was a Polymer Laboratories PLRP-S Polymeric Reversed Phase column (catalogue number PL1912-3802) (2.1×150 mm, 8 μm particle size, 1000 Å) with a flow rate of 1 mL/min, using the following gradient: 0 min, 25% B; 3 min, 25% B; 28 min, 50% B. Eluent A consisted of 0.05% trifluoroacetic acid (TFA) in water, eluent B of 0.05% trifluoroacetic acid in acetonitrile.

The peaks detected were assigned by retention time comparison with the light chain (L0) and the heavy chain (H0) of the unconjugated antibody. Peaks which were detected exclusively in the conjugated sample were assigned to the light chain, with a toxophore (L1), and to the heavy chains, with one, two and three toxophores (H1, H2, H3).

The average loading of the antibody with toxophores was determined from the peak areas determined by integration as follows: the light-chain loading was calculated as the sum of the toxophore number weighted integration results of the peaks belonging to the light chains divided by the sum of the singularly weighted integration results of the peaks belonging to the light chains. The heavy-chain loading was calculated as the sum of the toxophore number weighted integration results of the peaks belonging to the heavy chains divided by the sum of the singularly weighted integration results of the peaks belonging to the heavy chains. The average drug load results therefrom as the twofold sum of light-chain loading and heavy-chain loading. In certain individual cases it may be impossible exactly to determine the toxophore loading, owing to co-elutions of certain peaks.

### B-11. Testing of the Antigen Binding of the ADC

The binding capacity of the binder to the target molecule was tested after coupling had taken place. The skilled worker knows of diverse methods for achieving this for example, the affinity of the conjugate can be tested by means of ELISA technology or surface plasmon resonance analysis (BIAcore™ measurements). The conjugate concentration can be measured by the skilled person using common methods for example, for antibody conjugates, by means of protein determination (see also Doronina et al.; Nature Biotechnol. 2003; 21:778-784 and Polson et al., Blood 2007; 1102:616-623).

### WORKING EXAMPLES

### Immunoconjugates

### Example 1

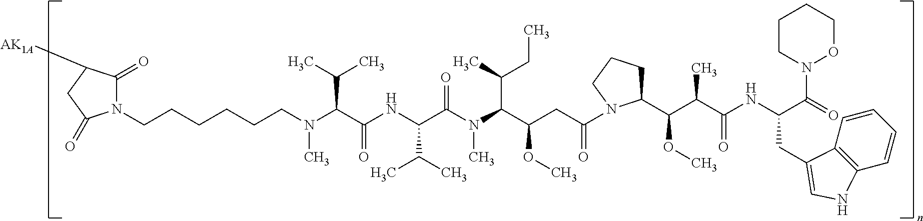

Coupling here was carried out using 30 mg of M048-D01-hIgG1 in PBS and following the Sephadex purification the batch was concentrated by ultracentrifugation, re-diluted with PBS and concentrated again.

Protein concentration: 15.5 mg/ml

### Drug/mAb Ratio: 3.7

### Example 2

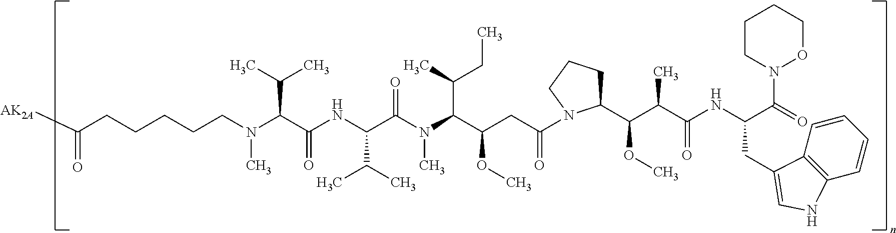

Coupling here was carried out using 32 mg of M048-D01-hIgG1 in PBS and following the Sephadex purification the batch was concentrated by ultracentifugation, re-diluted with PBS and concentrated again.

Protein concentration: 11.7 mg/ml

### Drug/mAb Ratio: 3.9

### Example 3

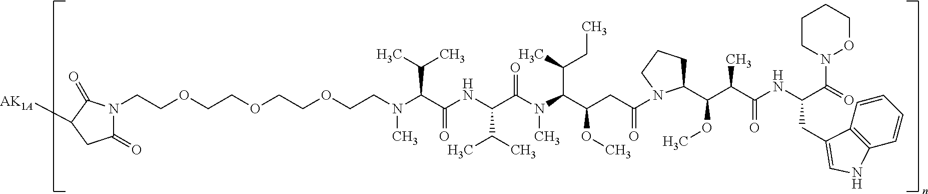

Coupling here was carried out using 30 mg of M048-D01-hIgG1 in PBS and following the Sephadex purification the batch was concentrated by ultracentrifugation, re-diluted with PBS and concentrated again.

Protein concentration: 12.5 mg/ml

### Drug/mAb Ratio: 3.7

### Example 4

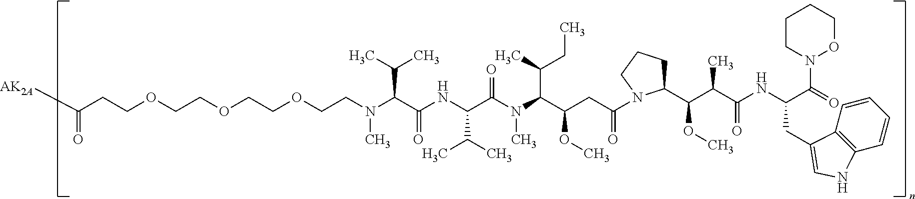

Coupling here was carried out using 2 mg of M048-D01-hIgG1 in PBS and following the Sephadex purification the batch was concentrated by ultracentifugation and re-diluted with PBS.

Protein concentration: 1.92 mg/ml

### Drug/mAb Ratio: 3.7

### Example 5

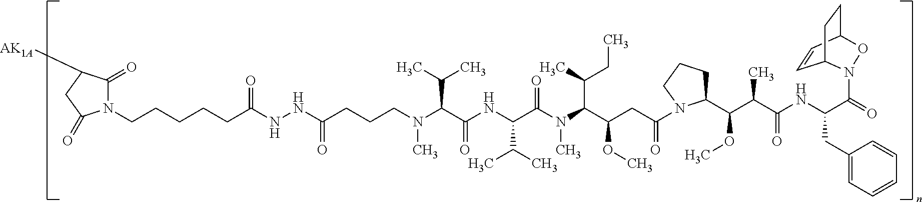

Coupling here was carried out using 3 mg of M048-D01-hIgG1 in PBS and following the Sephadex purification the batch was concentrated by ultracentrifugation and re-diluted.

Protein concentration: 2.54 mg/ml

### Drug/mAb Ratio: 3.1

### Example 6

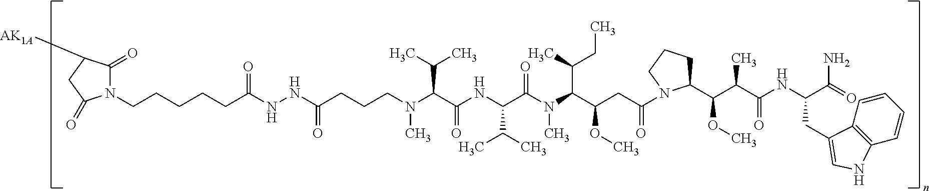

Coupling here was carried out using 4 mg of M048-D01-hIgG1 in PBS and following the Sephadex purification the batch was concentrated by ultracentrifugation and re-diluted.

Protein concentration: 1.53 mg/ml

### Drug/mAb Ratio: 2.7

### Example 7

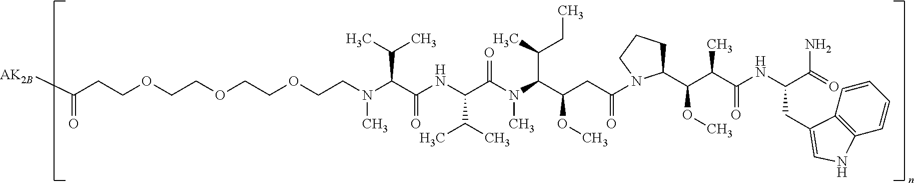

Coupling here was carried out using 5 mg of M048-D01-hIgG1-b in PBS and following the Sephadex purification the batch was concentrated by ultracentrifugation and re-diluted.

Protein concentration: 1.28 mg/ml

### Drug/mAb Ratio: 6.1

### Example 8

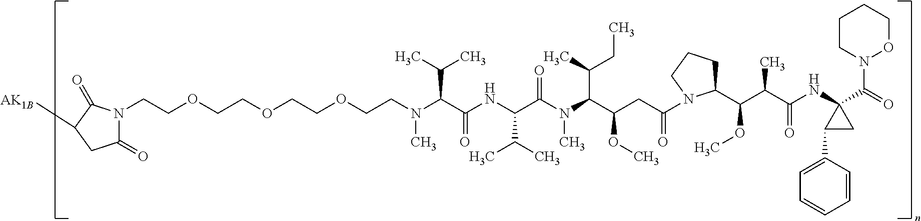

Coupling here was carried out using 5 mg of M048-D01-hIgG1-b in PBS and following the Sephadex purification the batch was concentrated by ultracentifugation and re-diluted.

Protein concentration: 1.26 mg/ml

### Drug/mAb Ratio: 3.5

### Example 9

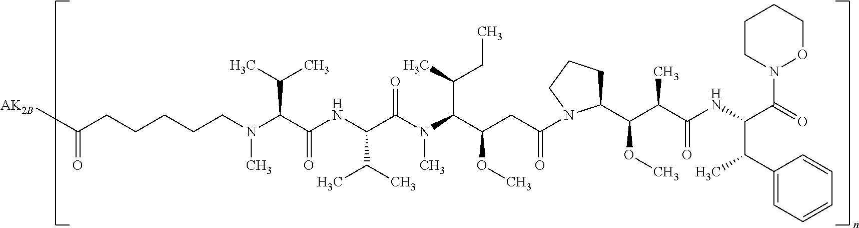

Coupling here was carried out using 5 mg of M048-D01-hIgG1-b in PBS and following the Sephadex purification the batch was concentrated by ultracentifugation and re-diluted.

Protein concentration: 1.28 mg/ml

### Drug/mAb Ratio: 6.1

### Example 10

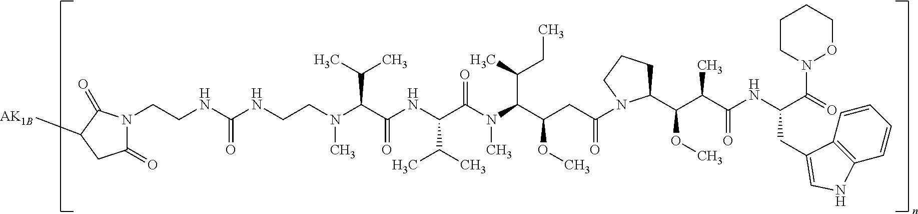

Coupling here was carried out using 5 mg of M048-D01-hIgG1-b in PBS and following the Sephadex purification the batch was concentrated by ultracentrifugation and re-diluted.

Protein concentration: 1.36 mg/ml

### Drug/mAb Ratio: 4.4

### Example 11

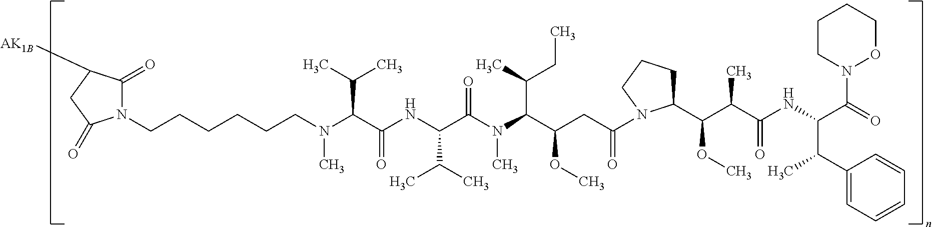

Coupling here was carried out using 5 mg of M048-D01-hIgG1-b in PBS and following the Sephadex purification the batch was concentrated by ultracentrifugation and re-diluted.

Protein concentration: 1.27 mg/ml

### Drug/mAb Ratio: 4.8

### Example 12

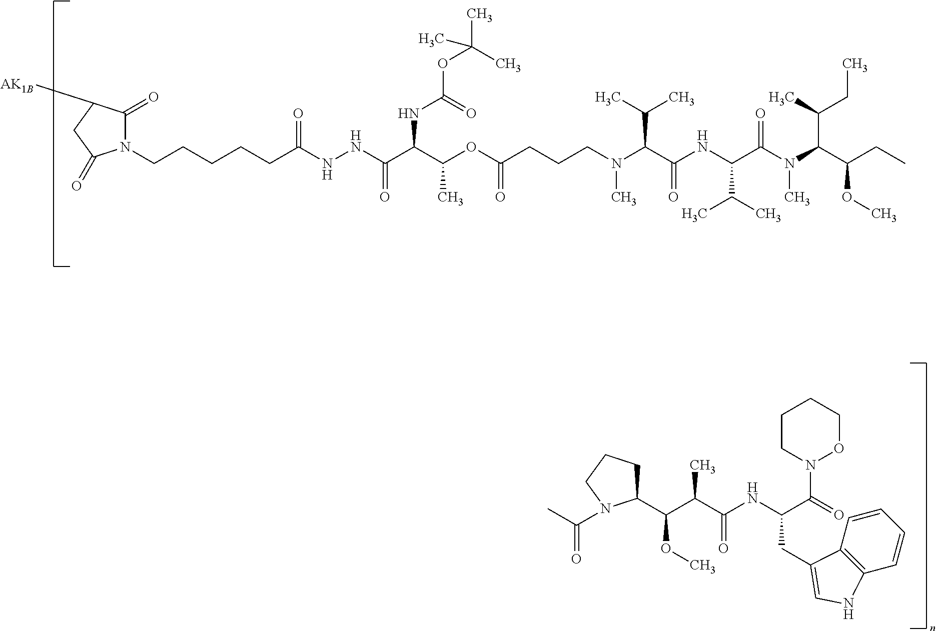

Coupling here was carried out using 5 mg of M048-D01-hIgG1-b in PBS and following the Sephadex purification the batch was concentrated by ultracentrifugation and re-diluted.

Protein concentration: 1.48 mg/ml

### Drug/mAb Ratio: 4.0

### Example 13

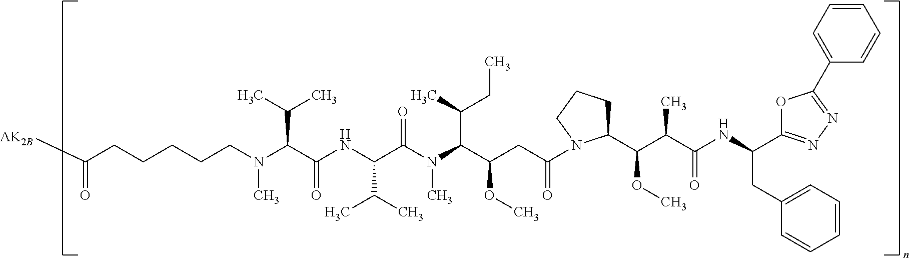

Coupling here was carried out using 5 mg of M048-D01-hIgG1-b in PBS and following the Sephadex purification the batch was concentrated by ultracentrifugation and re-diluted.

Protein concentration: 1.21 mg/ml

### Drug/mAb Ratio: 1.5

### Example 14

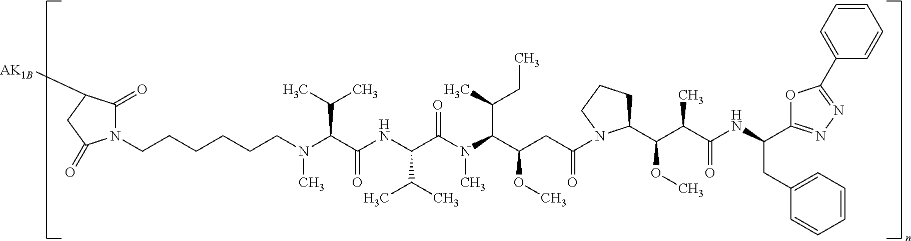

Coupling here was carried out using 5 mg of M048-D01-hIgG1-b in PBS and the batch, following Sephadex purification, was concentrated by ultracentrifugation and re-diluted.

Protein concentration: 1.27 mg/ml

### Drug/mAb Ratio: 2.7

### Example 15

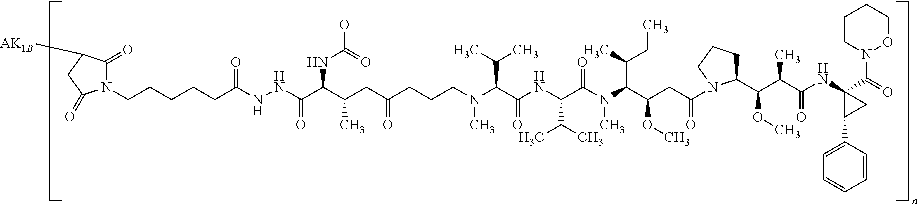

Coupling here was carried out using 5 mg of M048-D01-hIgG1-b in PBS and the batch, following Sephadex purification, was concentrated by ultracentrifugation and re-diluted with PBS.

Protein concentration: 1.37 mg/ml

### Drug/mAb Ratio: 3.9

### Example 16

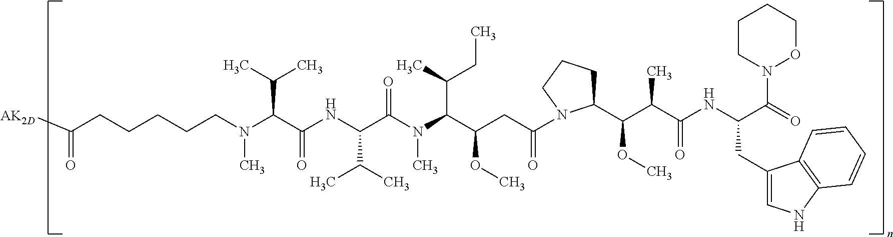

Coupling here was carried out using 4 mg of GAL-FR21-mIgG1 in PBS and the batch, following Sephadex purification, was concentrated by ultracentrifugation and re-diluted with PBS.

Protein concentration: 1.63 mg/ml

### Drug/mAb Ratio: 8.7

### Example 17

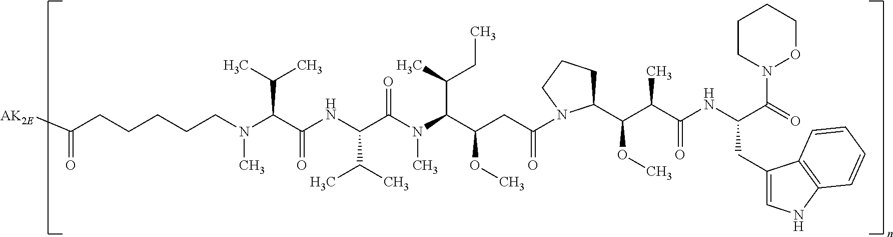

Coupling here was carried out using 4 mg of GAL-FR22-mIgG2a in PBS and the batch, following Sephadex purification, was concentrated by ultracentrifugation and re-diluted with PBS.

Protein concentration: 1.60 mg/ml

### Drug/mAb Ratio: 8.1

### Example 18

### N-(6-{[(5S)-5-Amino-5-carboxypentyl]amino}-6-oxohexyl)-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

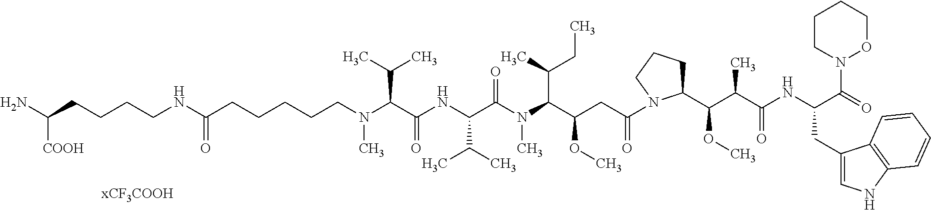 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCCCCC(=O)NCCCC[C@H](N)C(=O)O)C(C)C]

15.5 mg (15 μmol) of Intermediate 210 were taken up in 5 ml of DMF and admixed with 4.4 mg (18 μmol) of N²-(tert-butoxycarbonyl)-L-lysine and also 7.7 μL (44 μmol) of N,N-diisopropylethylamine. The reaction mixture was stirred at RT overnight and then concentrated under reduced pressure. The residue was subsequently purified by preparative HPLC. This gave 14 mg (81% of theory) of the protected intermediate of the title compound, which was subsequently taken up in 1 ml of dichloromethane and deprotected with 1 ml of trifluoroacetic acid. The batch was concentrated and, following lyophilization of the residue from acetonitrile/water (1:1), 15 mg (97% of theory) of the title compound were obtained.

HPLC (Method 12): Rₜ=1.8 min;

LC-MS (Method 1): Rₜ=0.79 min; MS (ESIpos): m/z=1083 (M+H)⁺.

### Example 19

### N-(6-{[(5S)-5-Amino-5-carboxypentyl]amino}-6-oxohexyl)-N-methyl-L-valyl-N-[(3R,4S,5S)-1-{(2S)-2-[(1R,2R)-3-{[(1S)-1-carboxy-2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

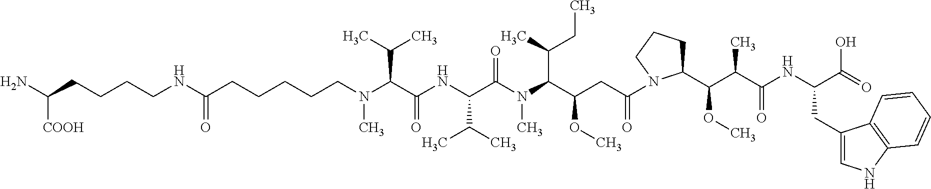 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)O)OC)N(C)C(=O)[C@@H](NC(=O)[C@H](C(C)C)N(C)CCCCCC(=O)NCCCC[C@H](N)C(=O)O)C(C)C]

40 mg (40 μmol) of Intermediate 227 were taken up in 5 ml of DMF and admixed with 11.5 mg (40 μmol) of N²-[(benzyloxy)carbonyl]-L-lysine and also 13 μL (80 μmol) of N,N-diisopropylethylamine. The reaction mixture was stirred at RT overnight, then concentrated under reduced pressure and subsequently purified by preparative HPLC. This gave 32.5 mg (70% of theory) of the protected intermediate of the title compound.

This 32.5 mg of the intermediate were dissolved in 10 ml of methanol and, following addition of 2 mg of 10% palladium on activated carbon, were hydrogenated under standard hydrogen pressure at RT for 30 minutes. The catalyst was then removed by filtration and the solvent was removed under reduced pressure. Lyophilization of the residue from dioxane/water 1:1 gave 26 mg (99% of theory) of the title compound.

HPLC (Method 12): Rₜ=1.7 min;

LC-MS (Method 1): Rₜ=0.76 min; MS (ESIpos): m/z=1014 (M+H)⁺.

### Example 20

### N-[(18S)-18-Amino-18-carboxy-12-oxo-3,6,9-trioxa-13-azaoctadec-1-yl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

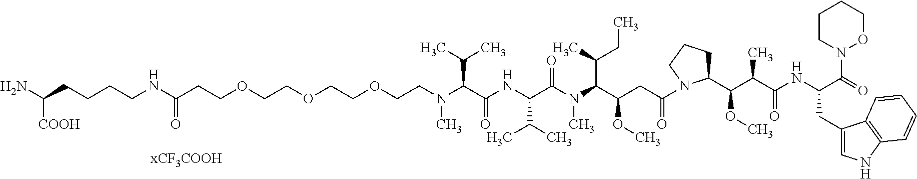 [SMILES: CC[C@H](C)[C@@H]([C@@H](CC(=O)N1CCC[C@H]1[C@H](OC)[C@@H](C)C(=O)N[C@@H](Cc1c[nH]c2ccccc12)C(=O)N1CCCCO1)OC)N(C)C(=O)[C@@H](NC(=O)[C@H](C(C)C)N(C)CCOCCOCCOCCC(=O)NCCCC[C@H](N)C(=O)O)C(C)C]

3.5 mg (3 μmol) of Intermediate 202 were taken up in 2 ml of DMF and admixed with 0.8 mg (3 μmol) of N²-(tert-butoxycarbonyl)-L-lysine and also 1.6 μL (10 μmol) of N,N-diisopropylethylamine. The reaction mixture was stirred at RT overnight and then concentrated under reduced pressure. The residue was taken up in acetonitrile/water (1:1), brought to a pH of 2 with trifluoroacetic acid and then purified by preparative HPLC. This gave 1 mg (25% of theory) of the protected intermediate of the title compound, which was subsequently taken up in 500 μl of dichloromethane and deprotected with 500 μl of trifluoroacetic acid. The batch was concentrated and, following lypophilization of the residue from acetonitrile/water (1:1), 1 mg (89% of theory) of the title compound was obtained.

HPLC (Method 12): Rₜ=1.9 min;

LC-MS (Method 1): Rₜ=0.82 min; MS (ESIpos): m/z=1173 (M+H)⁺.

### Example 21

### N-(4-{2-[6-(3-{[(2R)-2-Amino-2-carboxyethyl]sulphanyl}-2,5-dioxopyrrolidin-1-yl)hexanoyl]hydrazino}-4-oxobutyl)-N-methyl-L-valyl-N-[(3R,4S,5S)-1-{(2S)-2-[(1R,2R)-3-{[(2S)-1-amino-3-(1H-indol-3-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

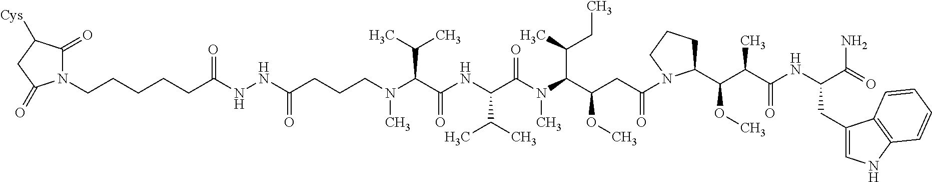

10 mg (10 μmol) of Intermediate 157 were taken up in 5.2 ml of DMF and admixed with 2.28 mg (20 μmol) of L-cysteine. The reaction mixture was stirred at RT for 2 hours, then concentrated under reduced pressure and subsequently purified by preparative HPLC. This gave 5.8 mg (48% of theory) of the title compound.

HPLC (Method 5): Rₜ=1.45 min;

LC-MS (Method 1): Rₜ=0.74 min; MS (ESIpos): m/z=1184 (M+H)⁺.

### Example 22

### N-(4-{2-[6-(3-{[(2R)-2-Amino-2-carboxyethyl]sulphanyl}-2,5-dioxopyrrolidin-1-yl)hexanoyl]hydrazino}-4-oxobutyl)-N-methyl-L-valyl-N-[(3R,4S,5S)-1-{(2S)-2-[(1R,2R)-3-{[(1S)-1-carboxy-2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

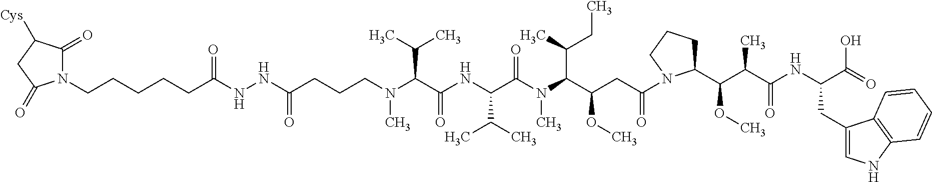

10 mg (10 μmol) of Intermediate 113 were taken up in 5.2 ml of DMF and admixed with 2.28 mg (20 μmol) of L-cysteine. The reaction mixture was stirred at RT for 2 hours, then concentrated under reduced pressure and subsequently purified by preparative HPLC. This gave 6 mg (54% of theory) of the title compound.

HPLC (Method 5): Rₜ=1.5 min;

LC-MS (Method 1): Rₜ=0.77 min; MS (ESIpos): m/z=1185 (M+H)⁺.

### Example 23

### N-[6-(3-{[(2R)-2-Amino-2-carboxyethyl]sulphanyl}-2,5-dioxopyrrolidin-1-yl)hexyl]-N-methyl-L-valyl-N-[(3R,4S,5S)-1-{(2S)-2-[(1R,2R)-3-{[(1S)-1-carboxy-2-(1H-indol-3-yl)ethyl]amino}-1-methoxy-2-methyl-3-oxopropyl]pyrrolidin-1-yl}-3-methoxy-5-methyl-1-oxoheptan-4-yl]-N-methyl-L-valinamide

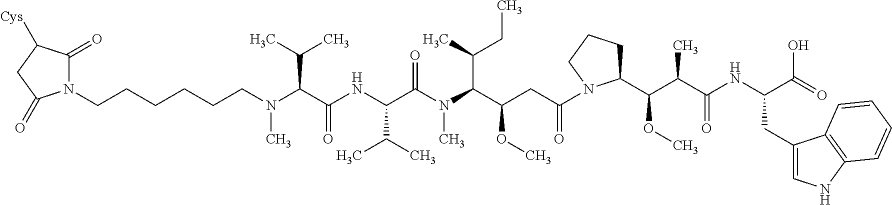

10 mg (10 μmol) of Intermediate 124 were taken up in 4 ml of DMF and admixed with 2.5 mg (20 μmol) of L-cysteine. The reaction mixture was stirred at RT for 2 hours, then concentrated under reduced pressure and subsequently purified by preparative HPLC. This gave 7.2 mg (64% of theory of the title compound.

HPLC (Method 5): Rₜ=1.6 min;

LC-MS (Method 1): Rₜ=0.8 min; MS (ESIpos): m/z=1071 (M+H)⁺.

### Example 24

### N-[6-(3-{[(2R)-2-Amino-2-carboxyethyl]sulphanyl}-2,5-dioxopyrrolidin-1-yl)hexyl]-N-methyl-L-valyl-N-[(3R,4S,5S)-1-{(2S)-2-[(1R,2R)-3-{[(2S)-3-(1H-indol-3-yl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide

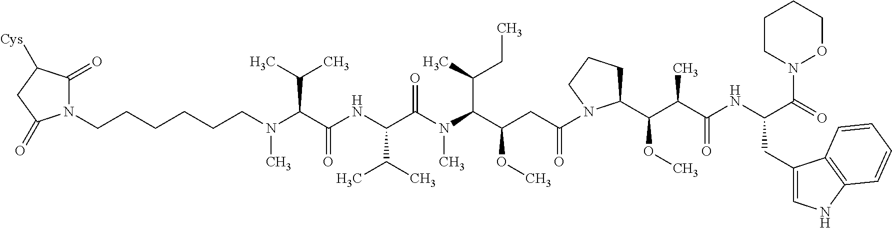

10 mg (10 μmol) of Intermediate 125 were taken up in 4 ml of DMF and admixed with 2.4 mg (20 μmol) of L-cysteine. The reaction mixture was stirred at RT for 2 hours, then concentrated under reduced pressure and subsequently purified by preparative HPLC. This gave 7.7 mg (69% of theory of the title compound.

HPLC (Method 5): Rₜ=1.7 min;

LC-MS (Method 2): Rₜ=1.91 min; MS (ESIpos): m/z=1140 (M+H)⁺.

### Example 25

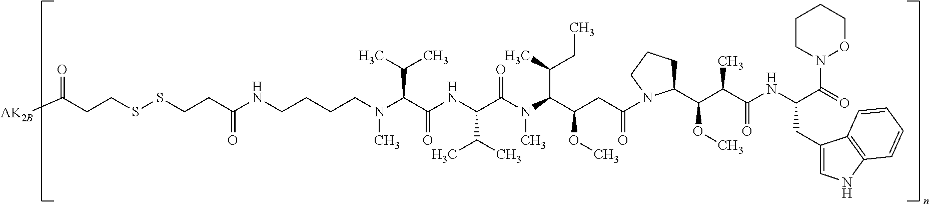

Coupling here was carried out using 5 mg of M048-D01-hIgG1-b in PBS and the batch, following Sephadex purification, was concentrated by ultracentrifugation and re-diluted.

Protein concentration: 1.27 mg/ml

### Drug/mAb Ratio: 1.0

### Example 26

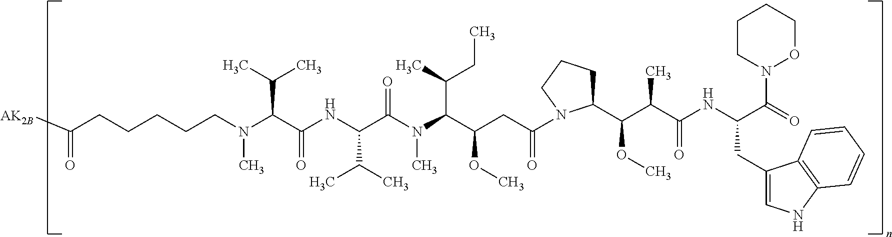

Coupling here was carried out using 35 mg of M048-D01-hIgG1-b in PBS and the batch, following Sephadex purification, was concentrated by ultracentrifugation, re-diluted with PBS and concentrated again.

Protein concentration: 11.60 mg/ml

### Drug/mAb Ratio: 3.7

### Example 27

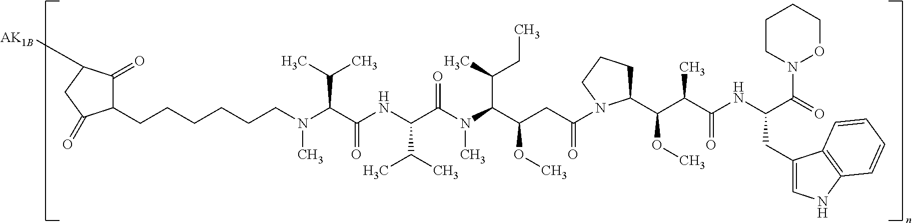

Coupling here was carried out using 35 mg of M048-D01-hIgG1-b in PBS and the batch, following Sephadex purification, was concentrated by ultracentrifugation, re-diluted with PBS and concentrated again.

Protein concentration: 11.7 mg/ml

### Drug/mAb Ratio: 4.2

### Example 28

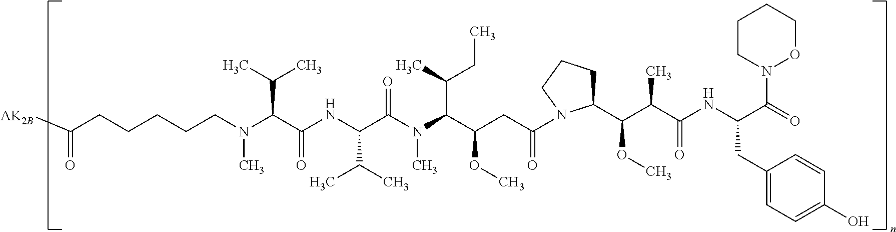

Coupling here was carried out using 5 mg of M048-D01-hIgG1-b in PBS and the reaction mixture, following Sephadex purification, was concentrated by ultracentrifugation and re-diluted with PBS.

Protein concentration: 1.31 mg/ml

### Drug/mAb Ratio: 3.7

### Example 29

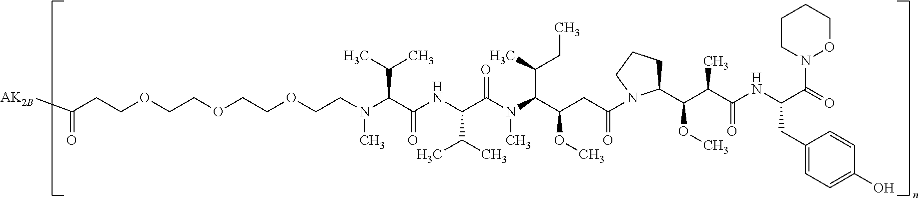

Coupling here was carried out using 5 mg of M048-D01-hIgG1-b in PBS and the reaction mixture, following Sephadex purification, was concentrated by ultracentrifugation and re-diluted with PBS.

Protein concentration: 1.53 mg/ml

### Drug/mAb Ratio: 1.3

### Example 30

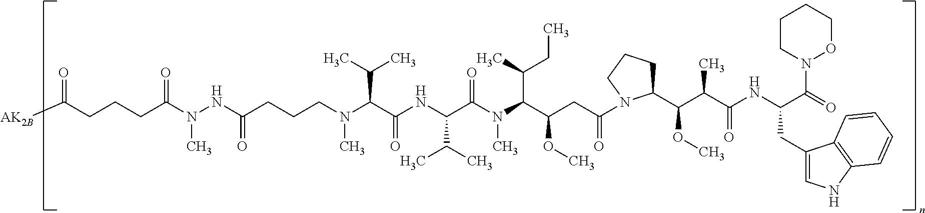

Coupling here was carried out using 5 mg of M048-D01-hIgG1-b in PBS and the batch, following Sephadex purification, was concentrated by ultracentrifugation and re-diluted.

Protein concentration: 1.61 mg/ml

### Drug/mAb Ratio: 3.9

### Example 31

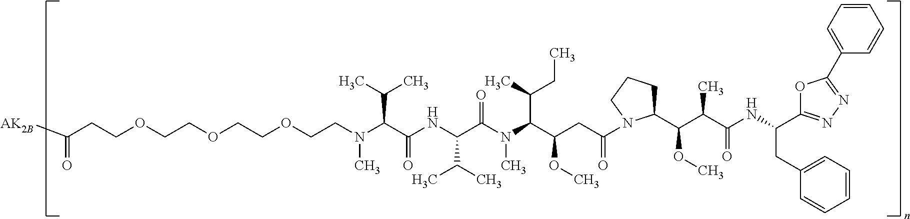

Coupling here was carried out using 5 mg of M048-D01-hIgG1-b in PBS and the reaction mixture, following Sephadex purification, was concentrated by ultracentrifugation and re-diluted.

Protein concentration: 1.32 mg/ml

### Drug/mAb Ratio: 1.7

### Example 32

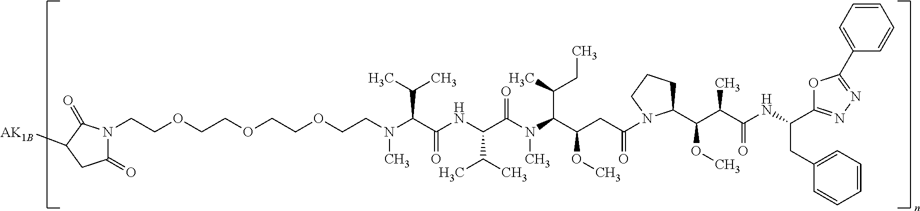

Coupling here was carried out using 5 mg of M048-D01-hIgG1-b in PBS and the reaction mixture, following Sephadex purification, was concentrated by ultracentrifugation and re-diluted.

Protein concentration: 1.12 mg/ml

### Drug/mAb Ratio: 0.2

### Example 33

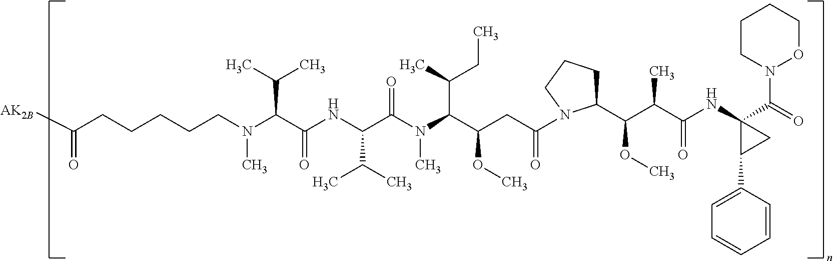

Coupling here was carried out using 5 mg of M048-D01-hIgG1-b in PBS and the reaction mixture, following Sephadex purification, was concentrated by ultracentrifugation and re-diluted.

Protein concentration: 1.29 mg/ml

### Drug/mAb Ratio: 5.5

### Example 34

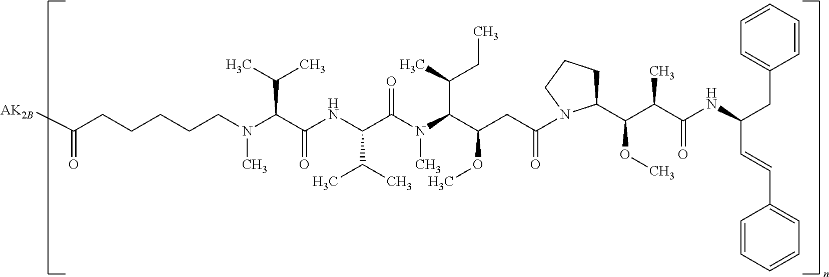

Coupling here was carried out using 5 mg of M048-D01-hIgG1-b in PBS and the reaction mixture, following Sephadex purification, was concentrated by ultracentrifugation and re-diluted.

Protein concentration: 1.04 mg/ml

### Drug/mAb Ratio: 0.5

### Example 35

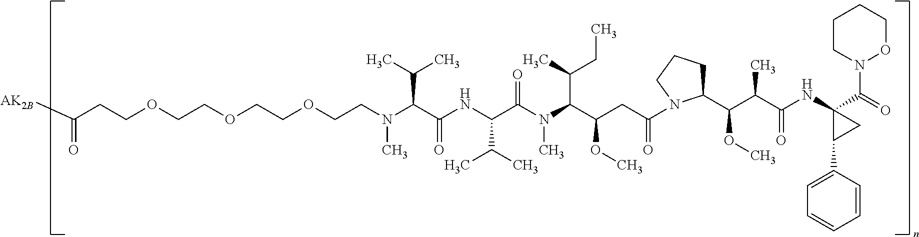

Coupling here was carried out using 5 mg of M048-D01-hIgG1-b in PBS and the reaction mixture, following Sephadex purification, was concentrated by ultracentrifugation and re-diluted.

Protein concentration: 1.66 mg/ml

### Drug/mAb-Ratio: 3.4

### Example 36

### N-(6-{[(5S)-5-Amino-5-carboxypentyl]amino}-6-oxohexyl)-N-methyl-L-valyl-N-[(3R,4S,5S)-1-{(2S)-2-[(1R,2R)-3-{[(2S)-3-(4-hydroxyphenyl)-1-(1,2-oxazinan-2-yl)-1-oxopropan-2-yl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

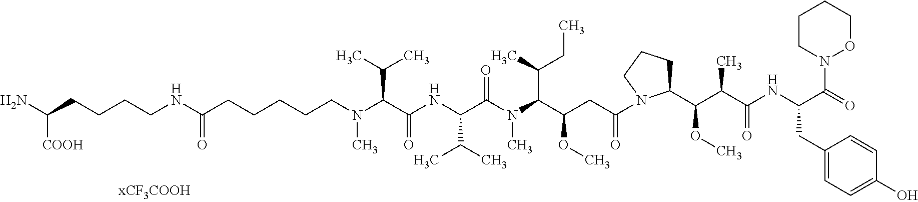 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)N1CCCCO1)OC)N(C)C(=O)[C@@H](NC(=O)[C@H](C(C)C)N(C)CCCCCC(=O)NCCCC[C@H](N)C(=O)O)C(C)C]

8 mg (8 μmol) of Intermediate 242 were taken up in 3 ml of DMF and admixed with 2.9 mg (12 μmol) of N²-(tert-butoxycarbonyl)-L-lysine and also 2.7 μL (16 μmol) of N,N-diisopropylethylamine. The reaction mixture was stirred at RT overnight, then admixed again with the same amounts of N²-(tert-butoxycarbonyl)-L-lysine and N,N-diisopropylethylamine, and stirred at RT for a further 4 hours. The batch was subsequently concentrated under reduced pressure. The residue was then purified by preparative HPLC. Lyophilization from acetonitrile/water gave 6.5 mg (72% of theory) of the protected intermediate of the title compound, which was subsequently taken up in 5 ml of dichloromethane and deprotected with 0.75 ml of trifluoroacetic acid. The batch was concentrated, and lyophilization of the residue from dioxane/water gave 5 mg (76% of theory) of the title compound.

HPLC (Method 12): Rₜ=1.7 min;

LC-MS (Method 1): Rₜ=0.69 min; MS (ESIpos): m/z=1059 (M+H)⁺.

### Example 37

### N-(6-{[(5S)-5-Amino-5-carboxypentyl]amino}-6-oxohexyl)-N-methyl-L-valyl-N-[(3R,4S,5S)-1-{(2S)-2-[(1R,2R)-3-{[(1S)-1-carboxy-2-(4-hydroxyphenyl)ethyl]amino}-1-methoxy-2-methyl-3-oxopropyl]pyrrolidin-1-yl}-3-methoxy-5-methyl-1-oxoheptan-4-yl]-N-methyl-L-valinamide trifluoroacetate

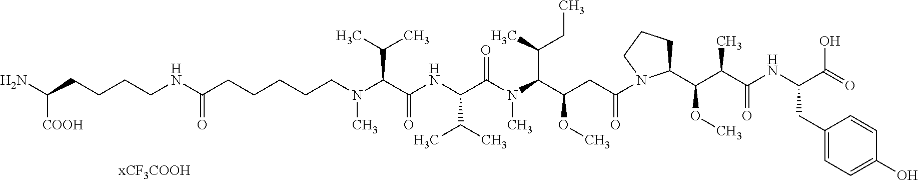 [SMILES: CC[C@H](C)[C@@H]([C@@H](CC(=O)N1CCC[C@H]1[C@H](OC)[C@@H](C)C(=O)N[C@@H](Cc1ccc(O)cc1)C(=O)O)OC)N(C)C(=O)[C@@H](NC(=O)[C@H](C(C)C)N(C)CCCCCC(=O)NCCCC[C@H](N)C(=O)O)C(C)C]

38 mg (41 μmol) of Intermediate 248 were first converted into the N-hydroxysuccinimide ester. 72 mg of the crude product obtained were taken up in 5 ml of DMF and admixed with 24 mg (100 μmol) of N²-(tert-butoxycarbonyl)-L-lysine and 23 μL of N,N-diisopropylethylamine. The reaction mixture was stirred at RT overnight, and then admixed again with 16 mg of N²-(tert-butoxycarbonyl)-L-lysine and 12 μL of N,N-diisopropylethylamine, and subsequently treated in an ultrasound bath for a further 2 hours. The batch was then concentrated under reduced pressure and the residue was purified by preparative HPLC. Lyophilization from acetonitrile/water gave 20 mg (50% of theory) of the protected intermediate of the title compound.

15 mg (12 μmol) of this intermediate were subsequently taken up in 3 ml of dichloromethane and admixed with 1 ml of trifluoroacetic acid. After 40 minutes of stirring at RT, a further 1.5 ml of trifluoroacetic acid were added and the batch was treated in an ultrasound bath for 1 hour. Thereafter the batch was concentrated, and lyophilization of the residue from dioxane/water gave 13 mg (90% of theory) of the title compound.

HPLC (Method 12): Rₜ=1.5 min;

LC-MS (Method 1): Rₜ=0.68 min; MS (ESIpos): m/z=990 (M+H)⁺.

### C: EVALUATION OF BIOLOGICAL ACTIVITY

The biological effect of the compounds of the invention was demonstrated by the assays described below:

C-1. Identification of Tumour Cells with Different FGFR2 Levels on the Cell Surface

To determine the amounts of FGFR2 available for the antibody on the cell surface, the cell binding of the FGFR2 antibody on different tumour cell lines was analysed in flow cytometry. The cell lines below were used for the experiments. The information on mutations and copy number of the FGFR2 comes from the Sanger Center Genome Project:
SNU-16 human stomach carcinoma cells, FGFR2 gene amplification (copy number 14; ATCC-CRL-5974, RPMI 1640 (Biochrom FG1215)+10% FCS
KatoIII human stomach carcinoma cells, FGFR2 gene amplification (copy number 14 ATCC-TCP-1008; Iscove's (Biochrom FG0465)+20% FCS
SUM52-PE human breast cancer cells, FGFR2 gene amplification (copy number 14; Asterand Lot No.: 28062A1-6004; Ham's F12 (Biochrom FG0815)+5% FCS+10 mM Hepes buffer+1 μg/ml hydrocortisone+5 μg/ml insulin
MFM-223 human breast cancer cells, FGFR2 gene amplification (copy number 14; ECACC-98050130, MEM Earle (Biochrom F0315)+10% FCS+2 mM glutamine
NCI-H716 human colorectal carcinoma cells, FGFR2 gene amplification (copy number: 8; ATCC-CCL-251; RPMI 1640 (Biochrom FG1215)+10% FCS)
MDA-MB-231 human breast cancer cells (no FGFR2 gene amplification, copy number 3; ATCC-HTB-26, DMEM/HAM's F12 (Biochrom FG4815)+10% FCS

For the experiments, adherent cells were washed twice with PBS (without calcium (Ca²⁺) and magnesium (Mg²⁺) ions and detached using enzyme-free, PBS-based cell dissociation buffer (Invitrogen). Approximately 1×10⁵ cells per well were suspended in FACS buffer (PBS without Ca²⁺ and Mg²⁺ with 3% FCS (Biochrom)), followed by centrifugation (250 g, 5 min, 4° C.), and the supernatant was discarded. The cells were resuspended in antibody dilutions (5 μg/ml in 80 μl) in FACS buffer, and incubated on ice for 1 hour. Next, the cells were washed once with 100 μl of cold FACS buffer and 80 μl of 1:150 diluted secondary antibodies (PE-coupled goat anti-mouse IgG, Jackson Immuno Research #115-115-164 for GAL-FR21-mIgG1 and for GAL-FR22-mIgG2a, and PE-coupled goat anti-human IgG, Dianova #109-115-098 for M048-D01-hIgG1) were added.

Following incubation on ice for 1 hour, the cells were again washed with cold FACS buffer, resuspended in 100 μl of FACS buffer, and analysed in an FACS array flow cytometer (BD Biosciences). The results were calculated as the geometric average value of the cell population detected with FGFR2 antibody, minus the background fluorescence, which was measured by incubating the cell population only with the secondary antibody. The values were analysed by the following system: geometric average value FGFR2 antibody minus geometric average value only secondary antibody >10+, >100++, >1000+++, >10000++++: no signal. Values in the vicinity of the category limits are marked with ( ).

The binding of FGFR2 antibodies to tumour cells is indicated in Table 4:

TABLE 4

| | M048-D01- | GAL-FR21- | GAL-FR22- |
|---|---|---|---|
| | hIgG1 | mIgG1 | mIgG2a |
| MFM-223 | +++ | +++ | +++ |
| SNU-16 | ++(+) | ++(+) | ++ |
| MDA-MB-231 | — | nd¹⁾ | nd¹⁾ |

¹⁾nd: not determined

The FGFR2 antibodies detect FGFR2 on the cell surface of MFM-223 and SNU-16 cancer cells.

### C-2. Determination of the Cytotoxic Effect of the ADCs Directed Against FGFR2

The cytotoxic effect of the FGFR2 ADCs was determined on various cell lines with different expression quantities of FGFR2, as follows:

The cells were cultured according to a standard method, using the growth media indicated under C-1. For the implementation, the cells were detached with a solution of trypsin (0.05%) and EDTA (0.02%) in PBS (Biochrom AG #L2143), pelletized, resuspended in culture medium, counted, and seeded into a 96-well culture plate with a white base (Costar #3610) (at 75 μl/well, following cell counts per well: SNU-16: 3000; MFM-223: 7000; MDA-MB-231: 4000; SUM52-PE: 3000; NCI-H716: 3000; KatoIII: 3000) and incubated in an incubator at 37° C. under 5% carbon dioxide. After 24 hours, the antibody-drug conjugates in 25 μl of culture medium (four-fold concentration) were applied to the cells, giving final antibody-drug conjugate concentrations of 3×10⁻⁷ M to 3×10⁻¹¹ M on the cells (triplicates). The cells were then incubated in an incubator at 37° C. and 5% carbon dioxide. In a parallel plate, the cell vitality at the beginning of drug treatment (day 0) was determined using the Cell Titer Glow Luminescent Cell Viability Assay (Promega #G7573 and #G7571). For this, 100 μl of the substrate were added per cell batch, and the plates were then covered with aluminium foil, shaken at 180 rpm with a plate shaker for 2 minutes, left to stand on the laboratory bench for 8 minutes, and then measured using a luminometer (Victor X2, Perkin Elmer). The substrate detects the ATP content in the living cells, producing a luminescence signal whose level is directly proportional to the vitality of the cells. After 72 hours of incubation with the antibody-drug conjugates, the vitality was determined in these cells as well, using the Cell Titer Glow Luminescent Cell Viability Assay as described above. From the data measured, the IC₅₀ of growth inhibition was calculated in comparison to that on day 0, using the laboratory software MTS (developed by Schering AG and Bayer Business Services 1999-2009) on the basis of a 4-parameter adaptation.

Table 5 below lists the IC₅₀ values¹) of representative working examples from this assay:

TABLE 5

| | SNU- | MFM- | MDA- | SUM52- | NCI- | |
|---|---|---|---|---|---|---|
| | 16, | 223, | MB-231, | PE, | H716, | KatoIII, |
| | IC50, | IC50, | IC50, | IC50, | IC50, | IC50, |
| Exam- | 72 hr | 72 hr | 72 hr | 72 hr | 72 hr | 72 hr |
| ple | [nM] | [nM] | [nM] | [nM] | [nM] | [nM] |
| 1 | 0.214 | 0.0907 | >300 | | | |
| 2 | 1.19 | 0.86 | >300 | | | |
| 3 | 0.15 | 0.195 | >300 | | | |
| 4 | 0.567 | 1.47 | >300 | | | |
| 5 | 0.496 | 4.00 | >300 | | | |
| 6 | 0.497 | 6.85 | >300 | | | |
| 7 | 0.209 | 0.628 | >300 | | | |
| 8 | 2.06 | 8.67 | 256 | | | |
| 9 | 2.05 | 4.78 | 230 | | | |
| 10 | 0.519 | 10.3 | >300 | | | |
| 11 | 0.614 | 0.581 | 80.9 | | | |
| 12 | 0.389 | 0.145 | >300 | | | |
| 13 | 0.254 | 0.431 | 6.61 | | | |
| 14 | 0.914 | 1.91 | 67.5 | | | |
| 15 | 16.6 | 16.9 | >300 | | | |
| 16 | <0.03 | 0.0788 | >300 | | | |
| 17 | 0.0616 | 0.278 | >300 | | | |
| 26 | 0.455 | na | >248 | <0.097 | 4.69 | na |
| 27 | 0.159 | 0.109 | >248 | <0.053 | 0.517 | 0.198 |
| 28 | 0.277 | na | na | 0.101 | na | na |
| 29 | 1.70 | na | na | 0.106 | na | na |
| 30 | 1.84 | 9.16 | >248 | 0.244 | na | na |
| 31 | 5.88 | na | na | na | na | na |
| 32 | >300 | na | na | na | na | na |
| 33 | 8.50 | na | 128 | 0.104 | na | 0.920 |
| 34 | 7.41 | na | >248 | 3.37 | na | na |
| 35 | na | na | >248 | 0.105 | na | 0.523 |

¹⁾ The activity data reported relate to the working examples described in the present experimental section, with the drug/mAB ratios indicated. The values may possibly deviate for different drug/mAB ratios.

Working Example 1 inhibited the proliferation of the SNU-16 and MFM-223 cancer cell lines which expresses FGFR2 at the cell surface, with an IC50 in the subnanomolar concentration range. Working Example 1 inhibited the proliferation of the MDA-MB-231 cancer cell line which do not express FGFR2 at the cell surface, with an IC50 of 300 nM. As is apparent from the data, all tested antibody-drug conjugates (Working Examples 1-31, 33-35) selectively inhibit the proliferation of FGFR2-expressing cancer cell lines (SNU-16, MFM-223, SUM52-PE, KatoIII or NCI-H716).

### C-3. Determination of the Effect on Tubulin Polymerization

Cancer cells are denatured cells which frequently lead to the formation of tumours also as a result of increased cell division. Microtubuli form the spindle fibres of the spindle apparatus and are an essential constituent of the cell cycle. The regulated construction and breakdown of microtubuli allows the precise division of the chromosomes among the daughter cells, and constitutes a continuously dynamic process. Disruption to this dynamic process results in incorrect cell division and ultimately in cell death. The increased cell division of cancer cells, however, also makes them particularly sensitive towards spindle fibre poisons, which constitute a fixed constituent of chemotherapy. Spindle fibre poisons such as paclitaxel or epothilone lead to a sharply increased polymerization rate of the microtubuli, while vinca alkaloids or else monomethylauristatin E (MMAE) lead to a sharply reduced polymerization rate of the microtubuli. In both cases, the necessary dynamism of the cell cycle is critically disrupted.

Tubulin polymerization was investigated using the “Fluorescence-based Microtubule Polymerisation Assay Kit” from Cytoskeleton (Denver, Colo., USA; order number: BK011). With this assay, GTP is added to unpolymerized tubulin, allowing polymerization to take place spontaneously. The assay is based on the binding of the fluorophore 4′,6-diamidino-2-phenylindole (DAPI) to tubulin. Free and bound DAPI can be differentiated on the basis of different emission spectra. Since DAPI exhibits a significantly high affinity for polymerized tubulin in comparison to non-polymerized tubulin, the tubulin polymerization can be followed via the increase in the fluorescence of bound DAPI fluorophores.

For the implementation of this assay, the compounds of the invention, in solution in DMSO, were diluted from their initial concentration of 10 mM to 1 μM in water. In addition to the buffer control, paclitaxel, with a polymerization-increasing effect, and vinblastin, with a polymerization inhibiting effect, were run additionally as assay controls. Measurement was carried out using 96-well plates with a half base area. The kinetics of the tubulin polymerization were monitored in a Fluorimeter at 37° C. for 1 hour. The excitation wavelength was 355 nm, and emission was monitored at 460 nm. For the region of linear increase within the first 10 minutes, a calculation was made of the change in fluorescence per minute (ΔF/min), which represents the polymerization rate of the microtubuli. The potency of the test substances was quantified on the basis of their respective reduction of the polymerization rate. Table 6 below gives data for the influence of representative working examples on tubulin polymerization.

TABLE 6
Blockade of tubulin polymerization by selected
examples of toxophore variants.

| | Concentration | Tubulin polymerization in the |
|---|---|---|
| | of | presence of toxophore in [%]. |
| Working | toxophore | Tubulin polymerization rate at |
| example | [μM] | 1 μM MMAF set at 100% |
| MMAF | 1 | 100 |
| MMAF | 10 | 34 |
| MMAF | 100 | 0 |
| 18 | 1 | 45 |
| 18 | 10 | 1 |
| 19 | 1 | 80 |
| 19 | 10 | 14 |
| 20 | 1 | 60 |
| 20 | 10 | 0 |
| 21 | 1 | 88 |
| 21 | 10 | 25 |
| 22 | 1 | 109 |
| 22 | 10 | 27 |
| 24 | 1 | 121 |
| 24 | 10 | 35 |
| 36 | 1 | 88 |
| 36 | 10 | 21 |
| 37 | 1 | 90 |
| 37 | 10 | 17 |

The MMAF toxophore and the working examples inhibit tubulin polymerization as a function of their concentration. At 100 μM MMAF, the tubulin polymerization is inhibited completely. The compounds investigated in the context of the present invention result in a reduced polymerization rate of the microtubuli. Working Examples 18-21 inhibit the tubulin polymerization at 1 μM to 45-88% of the value measured for 1 μM MMAF.

### C-4. In Vitro Tests for Determining Cell Permeability

The cell permeability of a substance can be investigated by means of in vitro testing in a flux assay using Caco-2 cells [M. D. Troutman and D. R. Thakker, Pharm. Res. 20 (8), 1210-1224 (2003)]. For this purpose, the cells were cultured for 15-16 days on 24-well filter plates. For the determination of permeation, the respective working example was applied in a HEPES buffer to the cells either apically (A) or basally (B) and incubated for 2 hours. After 0 hours and after 2 hours, samples were taken from the cis and trans compartments. The samples were separated by HPLC (Agilent 1200, Böblingen, Germany) using reverse phase columns. The HPLC system was coupled via a Turbo Ion Spray Interface to a Triple Quadropol mass spectrometer API 4000 (Applied Biosystems Applera, Darmstadt, Germany). The permeability was evaluated on the basis of a Pₐₚₚ value, which was calculated using the formula published by Schwab et al. [D. Schwab et al., J. Med. Chem. 46, 1716-1725 (2003)].

Of critical importance for toxophores which are released intracellularly is the permeability from B to A [Pₐₚₚ (B-A)]: the lower this permeability, the longer the residence time of the working example in the cell following intracellular release, and hence also the longer the time available for interaction with the biochemical target (in this case: tubulin).

Table 7 below sets out permeability data for representative working examples from this assay:

TABLE 7

| Working | Pₐₚₚ (B-A) |
|---|---|
| example | [nm/s] |
| 18 | 2 |
| 19 | 1 |
| 21 | 2 |
| 22 | 2 |
| 23 | 1 |
| 36 | 1.5 |
| 37 | 0.9 |

The working examples exhibit a low permeability from B to A [Pₐₚₚ (B-A) and therefore have a long residence time in the CaCo-2 cells. In comparison, monomethylauristatin E (MMAE) and monomethylauristatin F (MMAF) in this test exhibit a Pₐₚₚ (B-A) value of 73 nm/s, and therefore have a significantly shorter residence time in the Caco-2 cells.

### C-5. In Vitro Tests for Determining the Substrate Properties for P-Glycoprotein (P-gp)

Many tumour cells express transporter proteins for drugs, and this frequently accompanies the development of resistance towards cytostatics. Substances which are not substrates of such transporter proteins, such as P-glycoprotein (P-gp) or BCRP, for example, could therefore exhibit an improved activity profile.

The substrate properties of a substance for P-gp (ABCB1) were determined by means of a flux assay using LLC-PK1 cells which overexpress P-gp (L-MDR1 cells) [A. H. Schinkel et al., J. Clin. Invest. 96, 1698-1705 (1995)]. For this purpose, the LLC-PK1 cells or L-MDR1 cells were cultured on 96-well filter plates for 3-4 days. For determination of the permeation, the respective test substance, alone or in the presence of an inhibitor (such as Ivermectin or Verapamil, for example), was applied in a HEPES buffer to the cells either apically (A) or basally (B) and incubated for 2 hours. After 0 hours and after 2 hours, samples were taken from the cis and trans compartments. The samples were separated by HPLC using reverse phase columns. The HPLC system was coupled via a Turbo Ion Spray Interface to a Triple Quadropole mass spectrometer API 3000 (Applied Biosystems Applera, Darmstadt, Germany). The permeability was evaluated on the basis of a Pₐₚₚ value which was calculated using the formula published by Schwab et al. [D. Schwab et al., J. Med. Chem. 46, 1716-1725 (2003)].

Of critical importance for toxophores which are released intracellularly is the permeability from B to A [Pₐₚₚ (B-A)]: the lower this permeability, the longer the residence time of the working example in the cell following intracellular release, and hence also the longer the time available for interaction with the biochemical target (in this case: tubulin).

Table 8 below lists permeability data for representative working examples from this assay, which was carried out in L-MDR1 cells:

TABLE 8

| Working | Pₐₚₚ (B-A) |
|---|---|
| example | [nm/s] |
| 18 | 6 |
| 19 | 4 |
| 21 | 3 |
| 22 | 4 |
| 23 | 4 |

The working examples exhibit a low permeability from B to A [Pₐₚₚ (B-A) and therefore have a long residence time in the L-MDR1 cells.

### C-6. Pharmacokinetics in the SNU-16 Tumour Model

Following intravenous administration of various ADCs, the plasma concentrations and tumour concentrations of ADC and also of potential metabolites are measured and the pharmacokinetic parameters such as clearance (CL), area under the curve (AUC) and half-life (t_{1/2}) are calculated.

### Analysis for Quantifying the Potentially Occurring Metabolites

The measurement of the compounds in plasma and tumour takes place following precipitation of the proteins with methanol, by means of high-pressure liquid chromatography (HPLC) coupled to a tandem mass spectrometer (MS).

For the processing of 100 μL of plasma, it is admixed with 400 μL of methanol and 10 μL of internal standard (ISTD, 50 ng/mL in methanol) and shaken for 10 seconds. After centrifuging for 5 minutes at 16 000 g, 250 μL of supernatant are transferred to an autosampler vial, which was made up with 250 μL of ammonium acetate buffer (AAC, 10 mM, pH 6.8) and shaken again.

For the processing of a tumour, it is admixed with 4 times the amount of methanol. In a Tissuelyser II (Quiagen), the sample is comminuted at 30 impacts per minute for 6 minutes and then centrifuged off at 16 000 g for 5 minutes. 50 μL of the supernatant are transferred to an autosampler vial and made up with 50 μL of ammonium acetate buffer (10 mM, pH 6.8) and with 5 μL of ISTD. After again being shaken, the tumour sample is ready for measurement.

The measurement of both matrix samples takes place, lastly, with the aid of an HPLC-coupled, atmospheric pressure ionization/tandem mass spectrometer by means of a Turbo Ion Spray Interface (TISP) on an API4000 instrument from SCIEX.

HPLC/LC-MSMS (TISP) analysis runs on an HP1100 pump (Agilent) with a Gemini column (5 μm C18 110 A, 50×3 mm, Phenomenex).

### C-7. Activity Test In Vivo

The activity of the conjugates of the invention was tested in vivo by means for example of xenograft models. The skilled person knows of methods in the prior art for testing the activity of a conjugate of the invention (see, for example, WO 2005081711; Polson et al., Cancer Res. 2009 Mar. 15; 69(6):2358-64). For this purpose, for example, rodents (e.g. mice) were implanted with a tumour cell line which expresses the target molecule of the binder. These tumour-carrying rodents were subsequently administered either a conjugate of the invention or a control antibody conjugate, or isotonic salt solution. Administration took place singly or more often. The tumour growth was determined twice a week by means of a sliding caliper. After a tumour growth of several weeks, the tumour size of conjugate-treated animals and the control group was compared. The conjugate-treated animals showed a significantly lower tumour size.

C-7a. Testing of ADCs in Experimental Tumours in the Mouse

Human tumour cells which express FGFR2 were inoculated subcutaneously into the flank of immunosuppressed mice, such as nude mice or SCID mice. 1-10 million cells are detached from the cell culture, centrifuged and resuspended with 100 μl of medium, 50% medium/50% Matrigel or 100% Matrigel. The cell suspension was injected beneath the skin of the mouse.

Within a few days, a tumour grew. Treatment began no earlier than at a tumour size of 20-25 mm² after establishment of a tumour.

Treatment with conjugate took place via the intravenous route into the caudal vein of the mouse. The conjugate was dissolved in PBS and is administered with a volume of 5-10 ml/kg.

The treatment scheme was governed by the pharmacokinetics of the antibody. As a standard, treatment took place three times following every fourth day or every seventh day. Treatment, however, may also be continued further, or a second cycle with three days of treatment may follow at a later point in time.

As a standard basis, 8 animals were used per treatment group. This number may be higher if particularly strong fluctuations in tumour growth or after treatment are anticipated. As well as the groups which receive the active substances, one group, as a control group, was treated only with the buffer, in accordance with the same scheme.

In the course of the experiment, the area of the tumour was measured regularly using a sliding caliper in two dimensions (length/width). The area of the tumour was calculated by means of the formula length× width.

At the end of the experiment, the tumours were removed and weighed. The ratio of the average tumour weights for the therapy group (T) to the control group (C) was expressed as T/C. If control and treatment groups finished at different times, then the T/C value was calculated using the tumour areas of the last common measurement of all treatment and control groups.

C-7b. Testing of FGFR2-ADC in the SNU-16 Xenograft Model in the Mouse

Two million SNU-16 stomach carcinoma cells were inoculated subcutaneously into the flank of female NODscid mice.

Intravenous treatment with the conjugates was commenced at an average tumour size of 20-30 mm² When the control groups had reached the maximum permissible size, these groups were ended. The experimental groups treated with FGFR2 conjugates were ended when the tumours began to grow again. The activity of the conjugates was determined on day 31, the last point in time at which the vehicle control was still in the experiment. All of the FGFR2 conjugates tested inhibited tumour growth in a dose-dependent manner. At a dose of 5 mg/kg, Example 1 achieved a T/C of 0.08, Example 3 a T/C of 0.06 and Example 26 a T/C of 0.10. For all of the animals treated, the tumour at this point in time was smaller than at the beginning of treatment (partial regression of the tumour). At a dose of 2.5 mg/kg, Example 26 achieved a T/C of 0.14. At this dose, Example 26 led to partial regression in 40% of the animals. At the dose of 1 mg/kg, Example 1 achieved a T/C of 0.15 and Example 3 of 0.36. At this dose as well, Example 1 leads to partial regression in all of the animals treated, whereas in the case of Example 3 no partial regressions were obtained. Significant anti-tumour effect in comparison to the control was achieved for all of the conjugates tested, up to a dose of 1 mg/kg. The corresponding control antibody-conjugates showed no activity at all in this model at the same doses.

### C7c Testing of FGFR2-ADC in the MFM-223 Xenograft Model in the Mouse

10 million MFM-223 breast carcinoma cells were inoculated subcutaneously into the flank of female NMRI nu/nu mice. These mice had been supplemented beforehand with estradiol pellets.

Intravenous treatment with the conjugates was commenced at an average tumour size of 30-35 mm² When the control groups had reached the maximum permissible size on day 40, all of the treatment groups were ended and the tumour weights were ascertained. Example 26, with a dose of 10 mg/kg, achieved a T/C of 0.09, a T/C of 0.13 at a dose of 5 mg/kg and a T/C of 0.26 at a dose of 1 mg/kg. Significant anti-tumour effect in comparison to the control was achieved for all three doses tested. The corresponding control antibody conjugate showed a significant non-specific effect in this model only at the dose of 10 mg/kg.

### C7d Testing of FGFR2-2 ADC in the NCI-H716 Xenograft Model in the Mouse

1.5 Million NCI-H716 Intestinal Carcinoma Cells were Inoculated Subcutaneously into the Flank of Female NMRI nu/nu Mice.

Intravenous treatment with the conjugates was commenced at an average tumour size of 25-30 mm². When the control groups had reached the maximum permissible size on day 36, all of the treatment groups were ended and the tumour weights were ascertained. Treatment with Example 26 resulted in a significant reduction in tumour weight, achieving a T/C of 0.24 at 5 mg/kg. At this dose, the corresponding control conjugate had no activity at all in this model.

### D. WORKING EXAMPLES FOR PHARMACEUTICAL COMPOSITIONS

The compounds of the invention can be converted as follows into pharmaceutical preparations:

i.v. Solution:

The compound of the invention is dissolved at a concentration below the saturation solubility in a physiologically tolerated solvent (e.g. isotonic saline solution, D-PBS, or a formulation with glycine and sodium chloride in citrate buffer with addition of polysorbate 80). The solution is subjected to sterile filtration and dispensed into sterile and pyrogen-free injection containers.

i.v. Solution:

The compounds of the invention can be converted into the administration forms cited. This can be accomplished in a known way by “mixing with” or “dissolving in” inert, non-toxic, pharmaceutically suitable excipients (e.g. buffer substances, stabilizers, solubilizers, preservatives). The following, for example, may be present amino acids (glycine, histidine, methionine, arginine, lysine, leucine, isoleucine, threonine, glutamic acid, phenylalanine and others), sugars and related compounds (glucose, saccharose, mannitol, trehalose, sucrose, mannose, lactose, sorbitol), glycerol, sodium salts, potassium, ammonium salts and calcium salts (e.g. sodium chloride, potassium chloride or disodiumhydrogenphosphate and many others), acetate/acetic acid buffer systems, phosphate buffer systems, citric acid and citrate buffer systems, trometamol (TRIS and TRIS salts), Polysorbates (e.g. Polysorbate 80 and Polysorbate 20), Poloxamers (e.g. Poloxamer 188 and Poloxamer 171), Macrogols (PEG derivatives, e.g. 3350), Triton X-100, EDTA salts, glutathione, albumins (e.g. human), urea, benzyl alcohol, phenol, chlorocresol, metacresol, benzalkonium chloride and many others.

Lyophilizate for Subsequent Conversion into an i.v., s.c. or i.m. Solution:

Alternatively the compounds of the invention may be converted into a stable lyophilizate (possibly with the aid of abovementioned excipients) and, before being administered, reconstituted with a suitable solvent (e.g. injection-grade water, isotonic saline solution) and administered.

1. Binder-drug conjugates of the general formula (Ia) 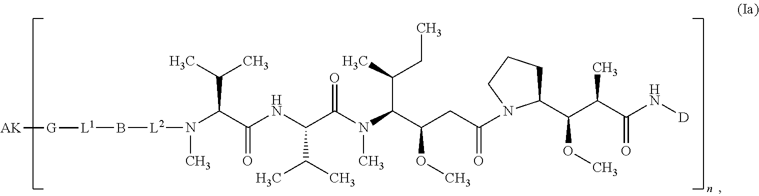
in which
n is a number from 1 to 50,
AK is a binder which binds to FGFR2,
the group §-G-L¹-B-§§ is a linker,
where
§ marks the linkage site with the group AK and
§§ marks the linkage site with the nitrogen atom,
L² is linear (C₂-C₁₀)-alkanediyl or is a group of the formula 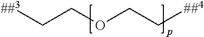
where
P is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2-, 1,3- or 1,4-relation to one another, with inclusion of any carbon atoms situated between them, may be bridged to form a (C₃-C₆)-cycloalkyl ring or a phenyl ring,
D is a group of the formula 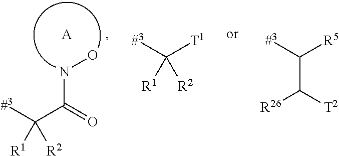
where
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula 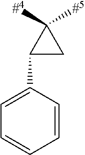
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 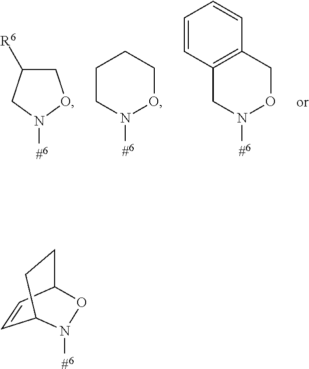
in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula 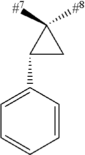
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula 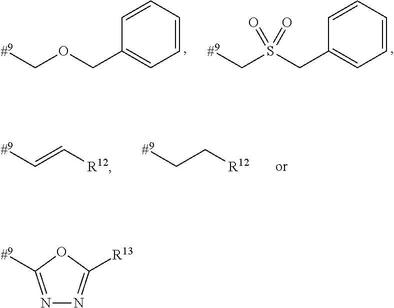
in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
R³⁵ is methyl or hydroxy,
and also their salts, solvates and solvates of the salts.

2. Binder-drug conjugates of the general formula (Ia) according to claim 1, in which
n is a number from 1 to 50,
AK is AK₁ or AK₂
where
AK₁ is a binder which binds to FGFR2 and is bonded via a sulphur atom of the binder to the group G,
AK₂ is a binder which binds to FGFR2 and is bonded via a nitrogen atom of the binder to the group G,
G when AK=AK₁, is a group of the formula 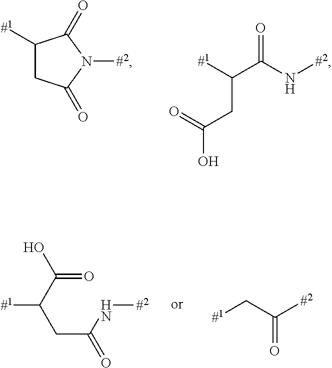
where
#¹ marks the linkage site with the sulphur atom of the binder,
#² marks the linkage site with the group L¹,
or
when AK=AK₂, is carbonyl,
L¹ is a bond, linear (C₁-C₁₀)-alkanediyl, a group of the formula 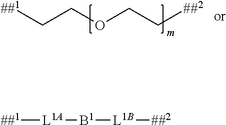
where
m is a number from 2 to 6,
##¹ marks the linkage site with the group G,
##² marks the linkage site with the group B,
L^{1a} is linear (C₂-C₁₀)-alkanediyl,
B¹ is a group of the formula 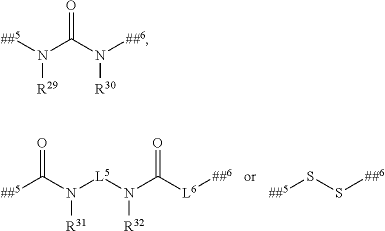
in which
##⁵ marks the linkage site with the group L^{1A},
##⁶ marks the linkage site with the group L^{1B},
L⁵ is a bond or (C₂-C₄)-alkanediyl,
L⁶ is a bond or a group of the formula 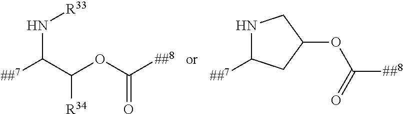
in which
##⁷ marks the linkage site with the carbonyl group,
##⁸ marks the linkage site with L^{1B},
R³³ is hydrogen, (C₁-C₄)-alkylcarbonyl, tert-butyloxycarbonyl or benzyloxycarbonyl,
R³⁴ is hydrogen or methyl,
R²⁹ is hydrogen or (C₁-C₄)-alkyl,
R³⁰ is hydrogen or (C₁-C₄)-alkyl,
or
R²⁹ and R³⁰ together with the atoms to which they are bonded form a 5- or 6-membered heterocycle,
R³¹ is hydrogen or (C₁-C₄)-alkyl,
R³² is hydrogen or (C₁-C₄)-alkyl,
or
R³¹ and R³² together with the atoms to which they are bonded form a 5- or 6-membered heterocycle,
L^{1B} is linear (C₂-C₁₀)-alkanediyl,
and
where (C₁-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
B is a bond or a group of the formula 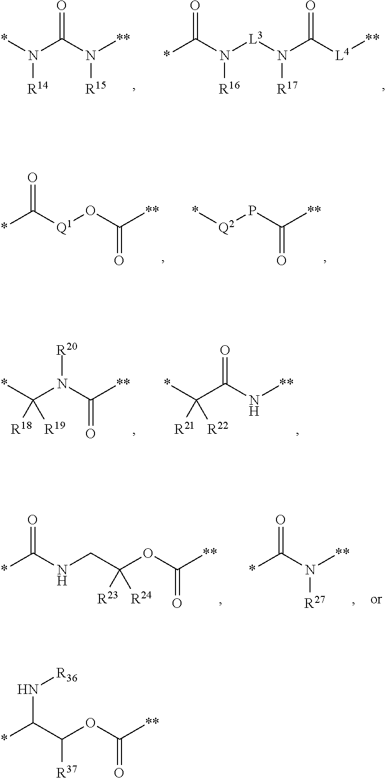
where
* marks the linkage site with L¹,
** marks the linkage site with L²,
P is O or NH,
L³ is a bond or (C₂-C₄)-alkanediyl,
L⁴ is a bond or a group of the formula 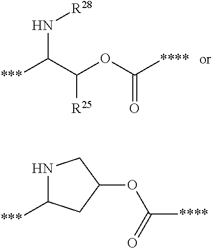
in which
*** marks the linkage site with the carbonyl group,
**** marks the linkage site with L²,
R²⁵ is hydrogen or methyl,
R²⁸ is hydrogen, (C₁-C₄)-alkylcarbonyl, tert-butyloxycarbonyl or benzyloxycarbonyl,
Q¹ is a 4- to 7-membered heterocycle,
Q² is a 3- to 7-membered carbocycle or a 4- to 7-membered heterocycle,
R¹⁴ is hydrogen or (C₁-C₄)-alkyl,
R¹⁵ is hydrogen or (C₁-C₄)-alkyl,
or
R¹⁴ and R¹⁵ together with the atoms to which they are bonded form a 5- or 6-membered heterocycle,
R¹⁶ is hydrogen or (C₁-C₄)-alkyl,
R¹⁷ is hydrogen or (C₁-C₄)-alkyl,
or
R¹⁶ and R¹⁷ together with the atoms to which they are bonded form a 5- or 6-membered heterocycle,
R¹⁸ is hydrogen or (C₁-C₄)-alkyl,
R¹⁹ is hydrogen or the side group of a natural □-amino acid or of its homologues or isomers,
R²⁰ is hydrogen or (C₁-C₄)-alkyl,
or
R¹⁹ and R²⁰ together with the atoms to which they are bonded form a pyrrolidinyl ring,
R²¹ is hydrogen or (C₁-C₄)-alkyl,
R²² is hydrogen or (C₁-C₄)-alkyl,
or
R²¹ and R²² together with the atoms to which they are bonded form a 3- to 7-membered carbocycle,
R²³ is (C₁-C₄)-alkyl,
R²⁴ is hydrogen or (C₁-C₄)-alkyl,
R²⁷ is hydrogen or (C₁-C₄)-alkyl,
R³⁶ is hydrogen, (C₁-C₄)-alkylcarbonyl, tert-butyloxycarbonyl or benzyloxycarbonyl,
R³⁷ is hydrogen or methyl,
or
R³⁶ and R³⁷ together with the atoms to which they are bonded form a pyrrolidine ring,
L² is linear (C₂-C₁₀)-alkanediyl or is a group of the formula 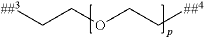
where
P is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2-, 1,3- or 1,4-relation to one another, with inclusion of any carbon atoms situated between them, may be bridged to form a (C₃-C₆)-cycloalkyl ring or a phenyl ring,
D is a group of the formula 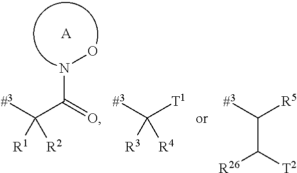
in which
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula 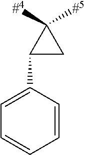
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 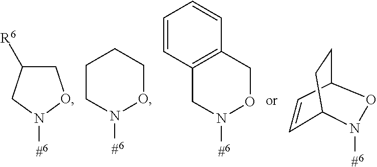
in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula 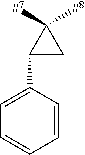
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula 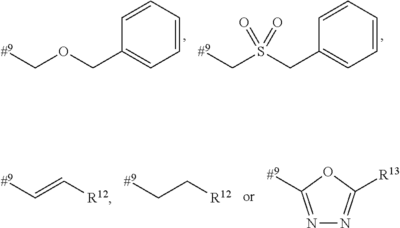
in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
R³⁵ is methyl or hydroxy,
and also their salts, solvates and solvates of the salts.

3. Binder-drug conjugates of the general formula (Ia) according to claim 1, in which
n is a number from 1 to 20,
AK is AK₁ or AK₂
where
AK₁ is a binder which binds to FGFR2 and is bonded via the sulphur atom of a cysteine residue of the binder to the group G,
AK₂ is a binder which binds to FGFR2 and is bonded via the NH side group of a lysine residue of the binder to the group G,
G when AK=AK₁, is a group of the formula 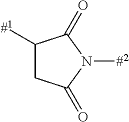
in which
#¹ marks the linkage site with the cysteine residue of the binder,
#² marks the linkage site with the group L¹,
or
when AK=AK₂, is carbonyl,
L¹ is a bond, linear (C₂-C₆)-alkanediyl, a group of the formula 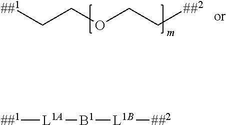
where
m is a number from 2 to 6,
##¹ marks the linkage site with the group G,
##² marks the linkage site with the group B,
L^{1A} is linear (C₂-C₆)-alkanediyl,
B¹ is a group of the formula 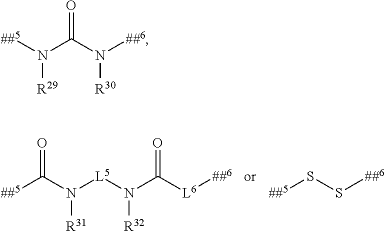
in which
##⁵ marks the linkage site with the group L^{1A},
##⁶ marks the linkage site with the group L^{1B},
L⁵ is a bond,
L⁶ is a bond or a group of the formula 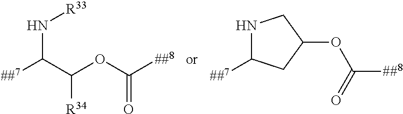
in which
##⁷ marks the linkage site with the carbonyl group,
##⁸ marks the linkage site with L^{1B},
R³³ is hydrogen, methylcarbonyl or tert-butyloxycarbonyl,
R³⁴ is hydrogen or methyl,
R²⁹ is hydrogen,
R³⁰ is hydrogen,
R³¹ is hydrogen or methyl,
R³² is hydrogen or methyl,
L^{1B} is linear (C₂-C₆)-alkanediyl,
and
where (C₂-C₆)-alkanediyl may be substituted by 1 or 2 methyl substituents,
B is a bond or a group of the formula 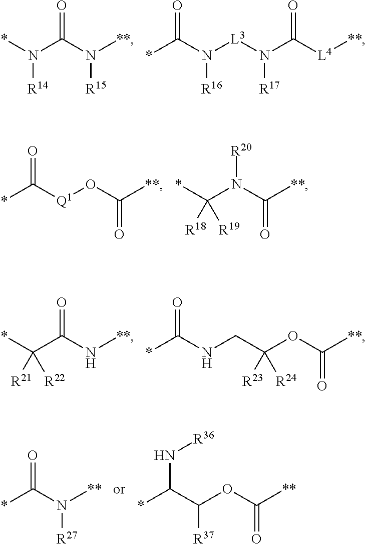
where
* marks the linkage site with L¹,
** marks the linkage site with L²,
L³ is a bond or ethane-1,2-diyl,
L⁴ is a bond or a group of the formula 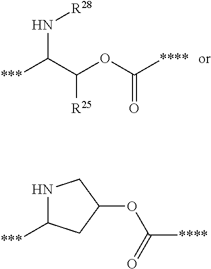
in which
*** marks the linkage site with the carbonyl group,
**** marks the linkage site with L²,
R²⁵ is hydrogen or methyl,
R²⁸ is hydrogen, methylcarbonyl or tert-butyloxycarbonyl,
Q¹ is a 4- to 7-membered heterocycle,
R¹⁴ is hydrogen,
R¹⁵ is hydrogen,
R¹⁶ is hydrogen or methyl,
R¹⁷ is hydrogen or methyl,
or
R¹⁶ and R¹⁷ together with the atoms to which they are bonded form a piperazinyl ring,
R¹⁸ is hydrogen,
R¹⁹ is hydrogen, methyl, propan-2-yl, 2-methylpropan-1-yl or 1-methylpropan-1-yl,
R²⁰ is hydrogen or methyl,
or
R¹⁹ and R²⁰ together with the atoms to which they are bonded form a pyrrolidinyl ring,
R²¹ is hydrogen or methyl,
R²² is hydrogen or methyl,
or
R²¹ and R²² together with the atoms to which they are bonded form a cyclopropyl ring,
R²³ is methyl,
R²⁴ is hydrogen or methyl,
R²⁷ is hydrogen,
R³⁶ is hydrogen, methylcarbonyl or tert-butyloxycarbonyl,
R³⁷ is hydrogen or methyl,
or
R³⁶ and R³⁷ together with the atoms to which they are bonded form a pyrrolidine ring,
L² is linear (C₂-C₆)-alkanediyl or is a group of the formula 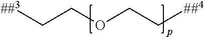
where
p is a number from 2 to 6,
##³ marks the linkage site with the group B,
##⁴ marks the linkage site with the nitrogen atom,
where (C₂-C₁₀)-alkanediyl may be substituted by 1 or 2 methyl substituents,
D is a group of the formula 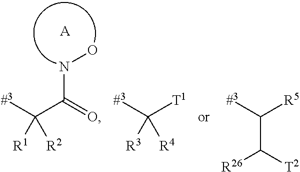
where
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula 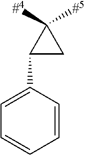
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 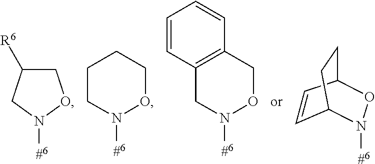
in which
#⁶ marks the linkage site with the carbonyl group,
R⁶ is hydrogen, hydroxy or benzyloxy,
R³ is hydrogen,
R⁴ is 1-hydroxyethyl, benzyl, 4-hydroxybenzyl, 1-phenylethyl or 1H-indol-3-ylmethyl,
or
R³ and R⁴ together with the carbon atom to which they are bonded form a (1S,2R)-2-phenylcyclopropane-1,1-diyl group of the formula 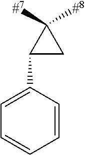
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula 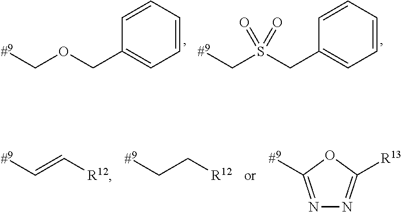
in which
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
R³⁵ is methyl or hydroxy,
and also their salts, solvates and solvates of the salts.

4. Binder-drug conjugates of the general formula (Ia) according to claim 1, in which
n is a number from 1 to 10,
AK is AK₁ or AK₂
where
AK₁ is a binder which binds FGFR2 and is bonded via the sulphur atom of a cysteine residue of the binder to the group G,
AK₂ is a binder which binds FGFR2 and is bonded via the NH side group of a lysine residue of the binder to the group G,
G when AK=AK₁, is a group of the formula 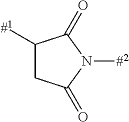
in which
#¹ marks the linkage site with the cysteine residue of the binder,
#² marks the linkage site with the group L¹,
or
when AK=AK₂, is carbonyl,
L¹ is a bond, linear (C₂-C₆)-alkanediyl, a group of the formula 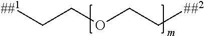
where
m is a number 2 or 3,
##¹ marks the linkage site with the group G,
##² marks the linkage site with the group B,
where (C₂-C₆)-alkanediyl may be substituted by 1 or 2 methyl substituents,
B is a bond or a group of the formula 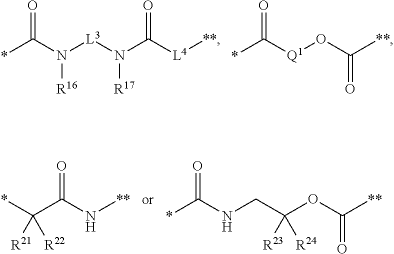
where
* marks the linkage site with L¹,
** marks the linkage site with L²,
L³ is a bond or ethane-1,2-diyl,
L⁴ is a bond or a group of the formula 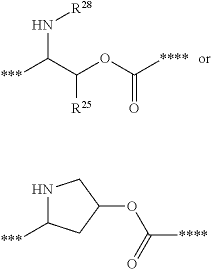
in which
*** marks the linkage site with the carbonyl group,
**** marks the linkage site with L²,
R²⁵ is methyl,
R²⁸ is hydrogen, methylcarbonyl or tert-butyloxycarbonyl,
Q¹ is piperidine-1,4-diyl,
R¹⁶ is hydrogen or methyl,
R¹⁷ is hydrogen or methyl,
or
R¹⁶ and R¹⁷ together with the atoms to which they are bonded form a piperazinyl ring,
R²¹ is hydrogen or methyl,
R²² is hydrogen or methyl,
or
R²¹ and R²² together with the atoms to which they are bonded form a cyclopropyl ring,
R²³ is methyl,
R²⁴ is hydrogen,
L² is linear (C₂-C₆)-alkanediyl or is a group of the formula 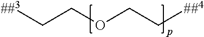
where
p is a number from 2 to 6,
##³ marks the linkage site with the group B,
##⁴ marks the linkage site with the nitrogen atom,
D is a group of the formula 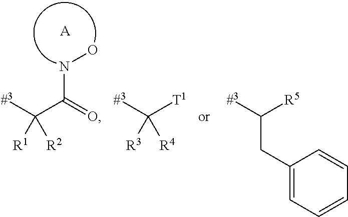
where
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula 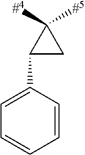
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 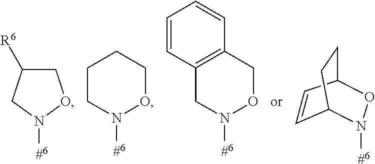
in which
#⁶ marks the linkage site with the carbonyl group,
R⁶ is hydrogen, hydroxy or benzyloxy,
R³ is hydrogen,
R⁴ is benzyl, 1-hydroxybenzyl, 1-phenylethyl or 1H-indol-3-ylmethyl,
or
R³ and R⁴ together with the carbon atom to which they are bonded form a (1S,2R)-2-phenylcyclopropane-1,1-diyl group of the formula 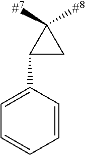
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
R¹¹ is benzyl, which may be substituted in the phenyl group by methoxycarbonyl or carboxyl,
R⁵ is hydrogen, methyl or a group of the formula 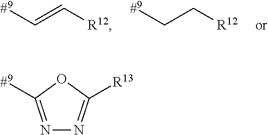
in which
#⁹ marks the linkage site with —CHCH₂phenyl,
R¹² is phenyl which may be substituted by methoxycarbonyl, carboxyl or a group of the formula —S(O)₂OH,
R¹³ is phenyl which may be substituted by methoxycarbonyl or carboxyl,
R³⁵ is methyl or hydroxy,
and also their salts, solvates and solvates of the salts.

5. Binder-drug conjugates of the general formula (Ia) according to claim 1, in which
n is a number from 1 to 10,
AK is AK₂
where
AK₂ is a binder which binds FGFR2 and is bonded via the NH side group of a lysine residue of the binder to the group G,
G is carbonyl,
L¹ is a bond,
B is a bond,
L² is linear (C₃-C₆)-alkanediyl or is a group of the formula 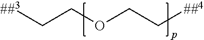
where
p is a number 2 or 3,
##³ marks the linkage site with the group B,
##⁴ marks the linkage site with the nitrogen atom,
D is a group of the formula 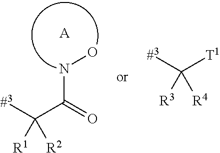
where
#³ marks the linkage site with the nitrogen atom,
R¹ is hydrogen,
R² is benzyl or 1H-indol-3-ylmethyl,
or
R¹ and R² together with the carbon atom to which they are bonded form a (1S,2R)-2-phenylcyclopropane-1,1-diyl group of the formula 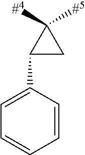
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 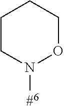
in which
#⁶ marks the linkage site with the carbonyl group,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula 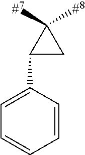
in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹ in which
R⁷ is hydrogen, methyl, ethyl, n-propyl, tert-butyl, benzyl or adamantylmethyl,
R⁸ is hydrogen,
R⁹ is hydrogen or benzyl,
R³⁵ is methyl,
and also their salts, solvates and solvates of the salts.

6. Binder-drug conjugates of the general formula (Ia) according to claim 1, in which
n is a number from 1 to 10,
AK is AK₁
where
AK₁ is a binder which binds FGFR2 and is bonded via the sulphur atom of a cysteine residue of the binder to the group G,
G is a group of the formula 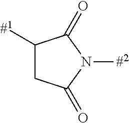
where
#¹ marks the linkage site with the cysteine residue of the binder,
#² marks the linkage site with the group L¹,
L¹ is a bond, linear (C₃-C₅)-alkanediyl or a group of the formula 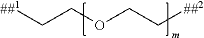
where
m is a number 2 or 3,
##¹ marks the linkage site with the group G,
##² marks the linkage site with the group B,
where (C₃-C₅)-alkanediyl may be substituted by 1 or 2 methyl substituents,
B is a bond or a group of the formula 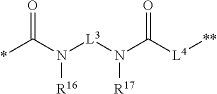
where
* marks the linkage site with L¹,
** marks the linkage site with L²,
L³ is a bond or ethane-1,2-diyl,
L⁴ is a bond or a group of the formula 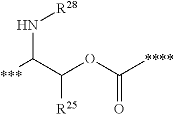
in which
*** marks the linkage site with the carbonyl group,
**** marks the linkage site with L²,
R²⁵ is methyl,
R²⁸ is hydrogen, methylcarbonyl or tert-butyloxycarbonyl,
R¹⁶ is hydrogen or methyl,
R¹⁷ is hydrogen or methyl,
or
R¹⁶ and R¹⁷ together with the atoms to which they are bonded form a piperazinyl ring,
L² is linear (C₃-C₅)-alkanediyl or is a group of the formula 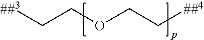
where
p is a number 2 or 3,
##³ marks the linkage site with the group B,
##⁴ marks the linkage site with the nitrogen atom,
D is a group of the formula 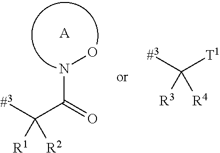
where
#³ marks the linkage site with the nitrogen atom,
R¹ is hydrogen,
R² is benzyl or 1H-indol-3-ylmethyl,
or
R¹ and R² together with the carbon atom to which they are bonded form a (1S,2R)-2-phenylcyclopropane-1,1-diyl group of the formula 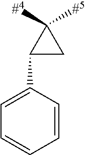
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 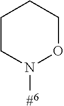
in which
#⁶ marks the linkage site with the carbonyl group,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula 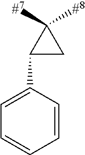
in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹,
in which
R⁷ is hydrogen, methyl, ethyl, n-propyl, tert-butyl, benzyl or adamantylmethyl,
R⁸ is hydrogen,
R⁹ is hydrogen or benzyl,
R³⁵ is methyl,
and also their salts, solvates and solvates of the salts.

7. Binder-drug conjugates of the general formula (Ia) 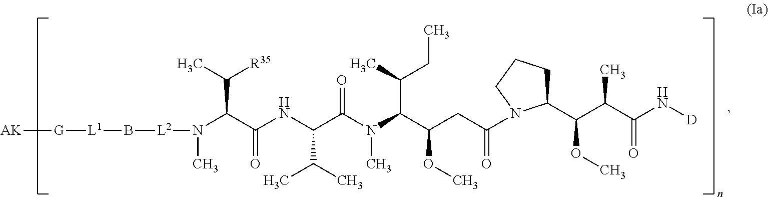
in which
n is a number from 1 to 50,
AK is a binder which binds to FGFR2,
the group §-G-L¹-B-§§ is a linker,
where
§ marks the linkage site with the group AK and
§§ marks the linkage site with the nitrogen atom,
L² is linear (C₂-C₁₀)-alkanediyl or is a group of the formula 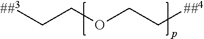
where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxyl and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
D is a group of the following formula, where * is the linkage site to the nitrogen atom 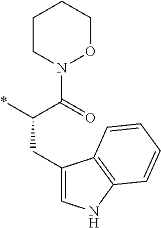 [SMILES: *[C@@H](Cc1c[nH]c2ccccc12)C(=O)N1CCCCO1]
and also their salts, solvates and solvates of the salts.

8. Compounds of the following formula 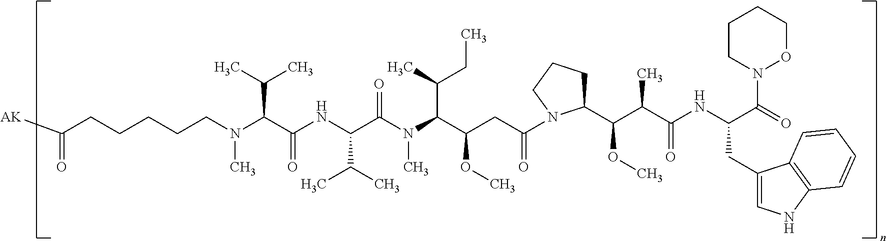
where AK is a binder which binds FGFR2, and n is a number from 1 to 10, and also their salts, solvates and solvates of the salts.

9. Compounds of the following formula 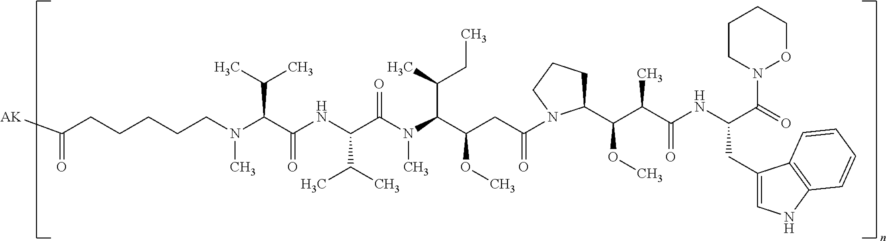
where AK is an antibody or an antibody fragment which binds FGFR2, and n is a number from 1 to 10, and also their salts, solvates and solvates of the salts.

10. Compound of the following formula 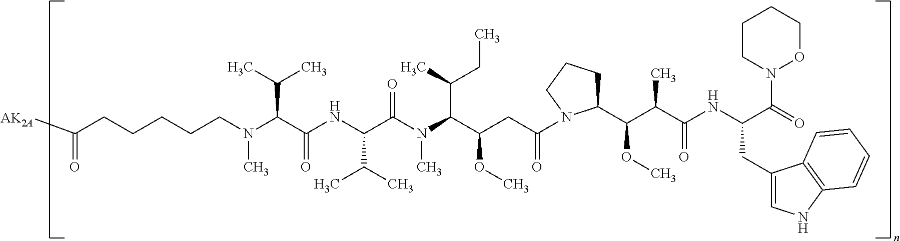
where AK2A is M048-D01-hIgG1 and n is a number from 1 to 10, and also their salts, solvates and solvates of the salts.

11. Compound of the following formula 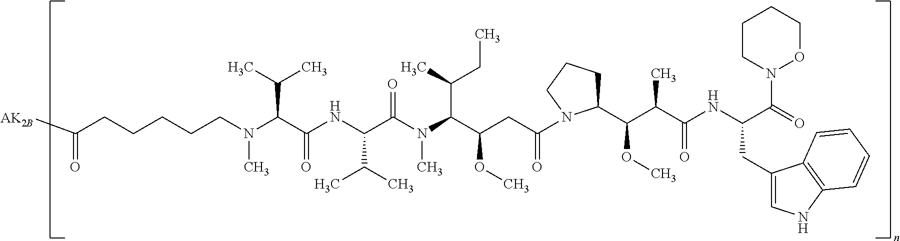
where AK2B is M048-D01-hIgG1-b and n is a number from 1 to 10, and also their salts, solvates and solvates of the salts.

12. Compounds of the formula (XXXa) 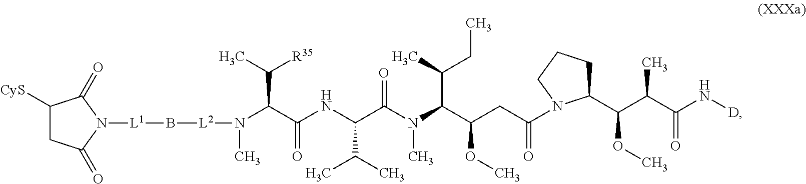
in which
Cys is a cysteine residue which is bonded via the sulphur atom of the side chain to a carbon atom of the succinimide,
L¹ is a bond, linear (C₁-C₁₀)-alkanediyl, a group of the formula 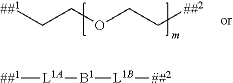
where
m is a number from 2 to 6,
##¹ marks the linkage site with the group G,
##² marks the linkage site with the group B,
L^{1A} is linear (C₂-C₁₀)-alkanediyl,
B¹ is a group of the formula 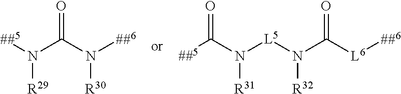
in which
##⁵ marks the linkage site with the group L^{1A},
##⁶ marks the linkage site with the group L^{1B},
L⁵ is a bond or (C₂-C₄)-alkanediyl,
L⁶ is a bond,
R²⁹ is hydrogen or (C₁-C₄)-alkyl,
R³⁰ is hydrogen or (C₁-C₄)-alkyl,
or
R²⁹ and R³⁰ together with the atoms to which they are bonded form a 5- or 6-membered heterocycle,
R³¹ is hydrogen or (C₁-C₄)-alkyl,
R³² is hydrogen or (C₁-C₄)-alkyl,
or
R³¹ and R³² together with the atoms to which they are bonded form a 5- or 6-membered heterocycle,
L^{1B} is linear (C₂-C₁₀)-alkanediyl,
and
where (C₁-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
B is a bond or a group of the formula 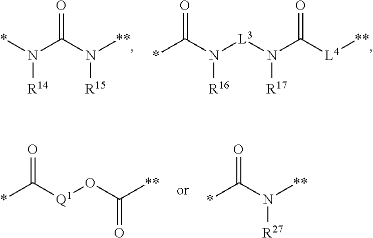
where
* marks the linkage site with L¹,
** marks the linkage site with L²,
P is O or NH,
L³ is a bond or (C₂-C₄)-alkanediyl,
L⁴ is a bond,
Q¹ is a 4- to 7-membered heterocycle,
Q² is a 3- to 7-membered carbocycle or a 4- to 7-membered heterocycle,
R¹⁴ is hydrogen or (C₁-C₄)-alkyl,
R¹⁵ is hydrogen or (C₁-C₄)-alkyl,
or
R¹⁴ and R¹⁵ together with the atoms to which they are bonded form a 5- or 6-membered heterocycle,
R¹⁶ is hydrogen or (C₁-C₄)-alkyl,
R¹⁷ is hydrogen or (C₁-C₄)-alkyl,
or
R¹⁶ and R¹⁷ together with the atoms to which they are bonded form a 5- or 6-membered heterocycle,
R¹⁸ is hydrogen or (C₁-C₄)-alkyl,
R¹⁹ is hydrogen or the side group of a natural □-amino acid or of its homologues or isomers,
R²⁰ is hydrogen or (C₁-C₄)-alkyl,
or
R¹⁹ and R²⁰ together with the atoms to which they are bonded form a pyrrolidinyl ring,
R²¹ is hydrogen or (C₁-C₄)-alkyl,
R²² is hydrogen or (C₁-C₄)-alkyl,
or
R²¹ and R²² together with the atoms to which they are bonded form a 3- to 7-membered carbocycle,
R²³ is (C₁-C₄)-alkyl,
R²⁴ is hydrogen or (C₁-C₄)-alkyl,
R²⁷ is hydrogen or (C₁-C₄)-alkyl,
L² is linear (C₂-C₁₀)-alkanediyl or is a group of the formula 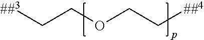
where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2-, 1,3- or 1,4-relation to one another, with inclusion of any carbon atoms situated between them, may be bridged to form a (C₃-C₆)-cycloalkyl ring or a phenyl ring,
D is a group of the formula 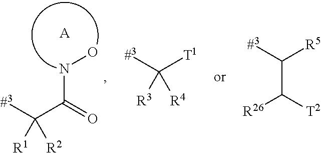
where
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula 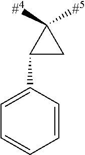
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 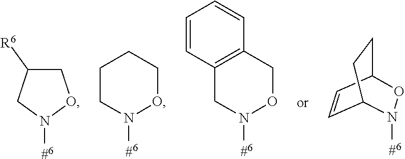
in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula 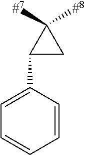
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula 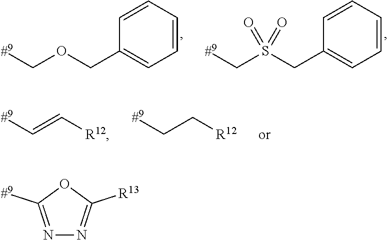
in which
#⁹ marks the linkage site with —CHC(R²⁶)-T²,
R¹² is phenyl which may be substituted by methoxycarbonyl, carboxyl or a group of the formula S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
R³⁵ is methyl or hydroxy,
and also their salts, solvates and solvates of the salts.

13. Compounds of the formula (XXXa) according to claim 12, in which
Cys is a cysteine residue which is bonded via the sulphur atom of the side chain via a carbon atom of the succinimide,
L¹ is a bond, linear (C₂-C₆)-alkanediyl, a group of the formula 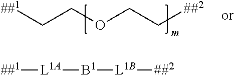
where
m is a number 2 or 3,
##¹ marks the linkage site with the group G,
##² marks the linkage site with the group B,
L^{1A} is linear (C₂-C₆)-alkanediyl,
B¹ is a group of the formula 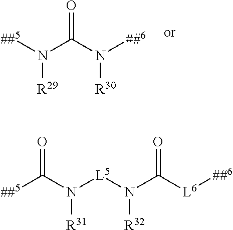
in which
##⁵ marks the linkage site with the group L^{1A},
##⁶ marks the linkage site with the group L^{1B},
L⁵ is a bond,
L⁶ is a bond,
R²⁹ is hydrogen,
R³⁰ is hydrogen,
R³¹ is hydrogen or methyl,
R³² is hydrogen or methyl,
L^{1B} is linear (C₂-C₆)-alkanediyl,
and
where (C₂-C₆)-alkanediyl may be substituted by 1 or 2 methyl substituents,
B is a bond or a group of the formula 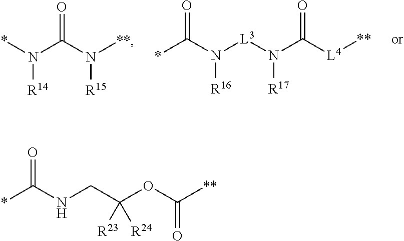
where
* marks the linkage site with L¹,
** marks the linkage site with L²,
L³ is a bond or ethane-1,2-diyl,
L⁴ is a bond,
R¹⁴ is hydrogen,
R¹⁵ is hydrogen,
R¹⁶ is hydrogen or methyl,
R¹⁷ is hydrogen or methyl,
or
R¹⁶ and R¹⁷ together with the atoms to which they are bonded form piperazinyl ring,
R²³ is methyl,
R²⁴ is hydrogen or methyl,
L² is linear (C₂-C₆)-alkanediyl or is a group of the formula 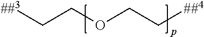
where
p is a number 2 or 3,
##³ marks the linkage site with the group B,
##⁴ marks the linkage site with the nitrogen atom,
D is a group of the formula 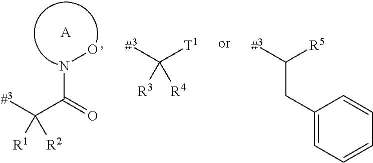
where
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula 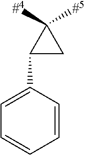
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 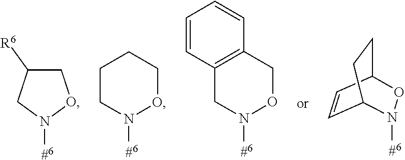
in which
#⁶ marks the linkage site with the carbonyl group,
R⁶ is hydrogen, hydroxy or benzyloxy,
R³ is hydrogen,
R⁴ is 1-hydroxyethyl, benzyl, 4-hydroxybenzyl, 1-phenylethyl or 1H-indol-3-ylmethyl,
or
R³ and R⁴ together with the carbon atom to which they are bonded form a (1S,2R)-2-phenylcyclopropane-1,1-diyl group of the formula 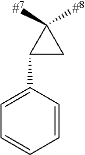
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl which may be substituted in the phenyl group by methoxycarbonyl or carboxyl,
R⁵ is hydrogen, methyl or a group of the formula 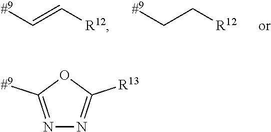
in which
#⁹ marks the linkage site to —CHCH₂phenyl,
R¹² is phenyl which may be substituted by methoxycarbonyl, carboxyl or a group of the formula —S(O)₂OH,
R¹³ is phenyl which may be substituted by methoxycarbonyl or carboxyl,
R³⁵ is methyl or hydroxy,
and also their salts, solvates and solvates of the salts.

14. Compounds of the formula (XXXa) according to claim 12, in which
Cys is a cysteine residue which is bonded via the sulphur atom of the side chain via a carbon atom of the succinimide,
L¹ is a bond or linear (C₂-C₆)-alkanediyl,
B is a bond or a group of the formula 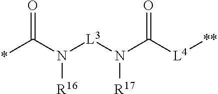
where
* marks the linkage site with L¹,
** marks the linkage site with L²,
L³ is a bond,
L⁴ is a bond,
R¹⁶ is hydrogen or methyl,
R¹⁷ is hydrogen or methyl,
L² is linear (C₂-C₆)-alkanediyl or is a group of the formula 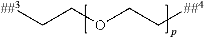
where
p is a number 2 or 3,
##³ marks the linkage site with the group B,
##⁴ marks the linkage site with the nitrogen atom,
D is a group of the formula 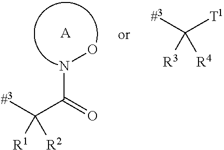
where
#³ marks the linkage site with the nitrogen atom,
R¹ is hydrogen,
R² is benzyl or 1H-indol-3-ylmethyl,
or
R¹ and R² together with the carbon atom to which they are bonded form a (1S,2R)-2-phenylcyclopropane-1,1-diyl group of the formula 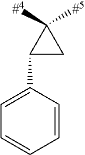
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 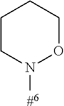
in which
#⁶ marks the linkage site with the carbonyl group,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula 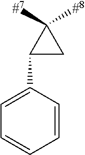
in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹,
in which
R⁷ is hydrogen,
R⁸ is hydrogen,
R⁹ is hydrogen,
R³⁵ is methyl,
and also their salts, solvates and solvates of the salts.

15. Compounds of the formula (XXXI) 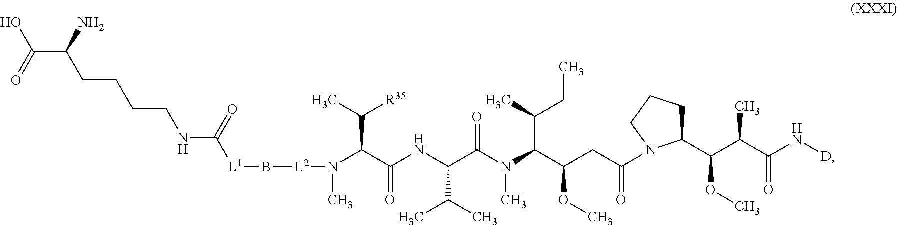
in which
L¹ is a bond, linear (C₁-C₁₀)-alkanediyl, a group of the formula 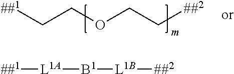
where
m is a number from 2 to 6,
##¹ marks the linkage site with the group G,
##² marks the linkage site with the group B,
L^{1A} is linear (C₂-C₁₀)-alkanediyl,
B¹ is a group of the formula 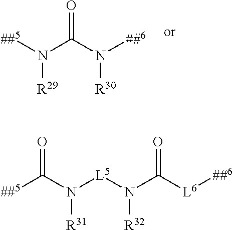
in which
##⁵ marks the linkage site with the group L^{1A},
##⁶ marks the linkage site with the group L^{1B},
L⁵ is a bond or (C₂-C₄)-alkanediyl,
L⁶ is a bond,
R²⁹ is hydrogen or (C₁-C₄)-alkyl,
R³⁰ is hydrogen or (C₁-C₄)-alkyl,
or
R²⁹ and R³⁰ together with the atoms to which they are bonded form a 5- or 6-membered heterocycle,
R³¹ is hydrogen or (C₁-C₄)-alkyl,
R³² is hydrogen or (C₁-C₄)-alkyl,
or
R³¹ and R³² together with the atoms to which they are bonded form a 5- or 6-membered heterocycle,
L^{1B} is linear (C₂-C₁₀)-alkanediyl,
and
where (C₁-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
B is a bond or a group of the formula 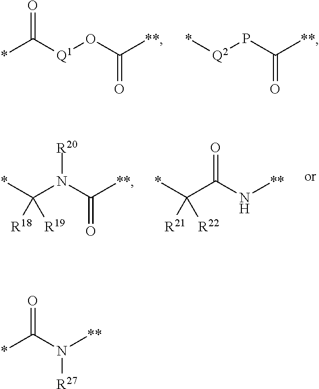
where
* marks the linkage site with L¹,
** marks the linkage site with L²,
P is O or NH,
Q¹ is a 4- to 7-membered heterocycle,
Q² is a 3- to 7-membered carbocycle or a 4- to 7-membered heterocycle,
R¹⁸ is hydrogen or (C₁-C₄)-alkyl,
R¹⁹ is hydrogen or the side group of a natural □-amino acid or of its homologues or isomers,
R²⁰ is hydrogen or (C₁-C₄)-alkyl,
or
R¹⁹ and R²⁰ together with the atoms to which they are bonded form a pyrrolidinyl ring,
R²¹ is hydrogen or (C₁-C₄)-alkyl,
R²² is hydrogen or (C₁-C₄)-alkyl,
or
R²¹ and R²² together with the atoms to which they are bonded form a 3- to 7-membered carbocycle,
R²⁷ is hydrogen or (C₁-C₄)-alkyl,
L² is linear (C₂-C₁₀)-alkanediyl or is a group of the formula 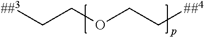
where
p is a number from 2 to 6,
##³ marks the linkage site with the group B,
##⁴ marks the linkage site with the nitrogen atom,
where (C₂-C₁₀)-alkanediyl may be substituted by 1 to 4 substituents selected independently of one another from the group consisting of methyl, hydroxy and benzyl,
and
where two carbon atoms of the alkanediyl chain in 1,2, 1,3 or 1,4-relation to one another, with inclusion of any carbon atoms situated between them, may be bridged to form a (C₃-C₆)-cycloalkyl ring or a phenyl ring,
D is a group of the formula 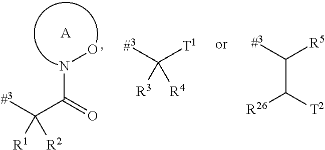
in which
#³ marks the linkage site with the nitrogen atom,
R¹ is hydrogen or methyl,
R² is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R¹ and R² together with the carbon atom to which they are bonded form a (1S,2R)-2-phenylcyclopropane-1,1-diyl group of the formula 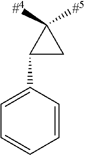
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 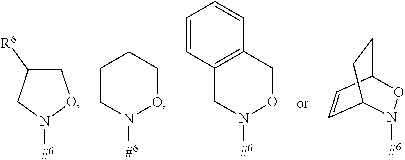
in which
#⁶ marks the linkage site with the carbonyl group,
R⁶ is hydrogen, hydroxy or benzyloxy,
R³ is hydrogen or methyl,
R⁴ is isopropyl, isobutyl, sec-butyl, tert-butyl, phenyl, benzyl, 1-hydroxyethyl, 4-hydroxybenzyl, 4-hydroxy-3-nitrobenzyl, 4-hydroxy-3-aminobenzyl, 1-phenylethyl, diphenylmethyl, 1H-imidazol-4-ylmethyl or 1H-indol-3-ylmethyl,
or
R³ and R⁴ together with the carbon atom to which they are bonded form a (1S,2R)-2-phenylcyclopropane-1,1-diyl group of the formula 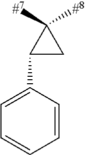
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula 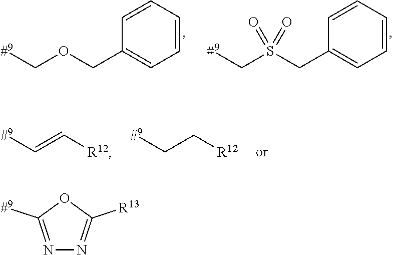
#⁹ marks the linkage site with —CHC(R²⁶)-T²,
R¹² is phenyl which may be substituted by methoxycarbonyl, carboxyl or a group of the formula —S(O)₂OH,
R¹³ is phenyl which may be substituted by methoxycarbonyl or carboxyl,
R²⁶ is hydrogen or hydroxy,
T² is phenyl, benzyl, 1H-indol-3-yl or 1H-indol-3-ylmethyl,
R³⁵ is methyl or hydroxy,
and also their salts, solvates and solvates of the salts.

16. Compounds of the formula (XXXI) according to claim 15, in which
L¹ is a bond, linear (C₂-C₆)-alkanediyl or a group of the formula 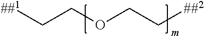
where
m is a number 2 or 3,
##¹ marks the linkage site with the group G,
##² marks the linkage site with the group B,
where (C₂-C₆)-alkanediyl may be substituted by 1 or 2 methyl substituents,
B is a bond or a group of the formula 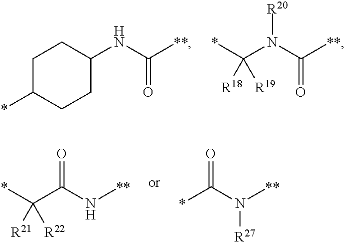
where
* marks the linkage site with L¹,
** marks the linkage site with L²,
R¹⁸ is hydrogen,
R¹⁹ is methyl, propan-2-yl, 2-methylpropan-1-yl or 1-methylpropan-1-yl,
R²⁰ is hydrogen or (C₁-C₄)-alkyl,
or
R¹⁹ and R²⁰ together with the atoms to which they are bonded form a pyrrolidinyl ring,
R²¹ is hydrogen or methyl,
R²² is hydrogen or methyl,
or
R²¹ and R²² together with the atoms to which they are bonded form a cyclopropyl ring,
R²⁷ is hydrogen or methyl,
L² is linear (C₂-C₆)-alkanediyl or is a group of the formula 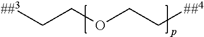
where
p is a number 2 or 3,
##³ marks the linkage site with the group B,
##⁴ marks the linkage site with the nitrogen atom,
where (C₂-C₁₀)-alkanediyl may be substituted by 1 or 2 methyl substituents, and
where two carbon atoms of the alkanediyl chain in 1,4-relation to one another, with inclusion of any carbon atoms situated between them, may be bridged to form a phenyl ring,
D is a group of the formula 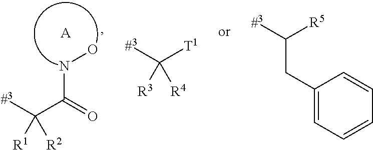
in which
#³ marks the linkage site with the nitrogen atom,
R¹ is hydrogen,
R² is 1-hydroxyethyl, benzyl, 4-hydroxybenzyl, 1-phenylethyl or 1H-indol-3-ylmethyl,
or
R¹ and R² together with the carbon atom to which they are bonded form a (1S,2R)-2-phenylcyclopropane-1,1-diyl group of the formula 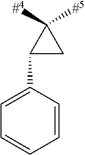
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 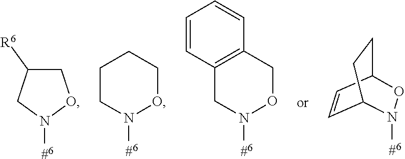
in which
#⁶ marks the linkage site with the carbonyl group,
R⁶ is hydrogen, hydroxy or benzyloxy,
R³ is hydrogen,
R⁴ is 1-hydroxyethyl, benzyl, 4-hydroxybenzyl, 1-phenylethyl or 1H-indol-3-ylmethyl,
or
R³ and R⁴ together with the carbon atoms to which they are bonded form a (1S,2R)-2-phenylcyclopropane-1,1-diyl group of the formula 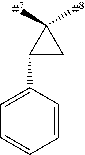
in which
#⁷ marks the linkage site with the adjacent nitrogen atom,
#⁸ marks the linkage site with the group T¹,
T¹ is a group of the formula —C(═O)—OR⁷, —C(═O)—NR⁸R⁹, —C(═O)—NH—NH—R¹⁰ or —CH₂—O—R¹¹,
in which
R⁷ is hydrogen, methyl, ethyl, n-propyl, tert-butyl, benzyl or adamantylmethyl,
R⁸ is hydrogen or methyl,
R⁹ is hydrogen, methyl, ethyl, n-propyl or benzyl,
or
R⁸ and R⁹ together with the nitrogen atom to which they are bonded form a 4- to 7-membered heterocycle,
R¹⁰ is benzoyl,
R¹¹ is benzyl, which may be substituted in the phenyl group by methoxycarbonyl or carboxyl,
R⁵ is hydrogen, methyl or a group of the formula 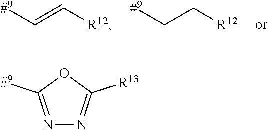
in which
#⁹ marks the linkage site with —CHCH₂phenyl,
R¹² is phenyl which may be substituted by methoxycarbonyl, carboxyl or a group of the formula —S(O)₂OH,
R¹³ is phenyl which may be substituted by methoxycarbonyl or carboxyl,
R³⁵ is methyl or hydroxy,
and also their salts, solvates and solvates of the salts.

17. Compounds of the formula (XXXI) according to claim 15, in which
L¹ is a bond,
B is a bond,
L² is linear (C₂-C₆)-alkanediyl or is a group of the formula 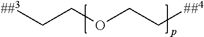
where
p is a number 2 or 3,
##³ marks the linkage site with the group B,
##⁴ marks the linkage site with the nitrogen atom,
D is a group of the formula 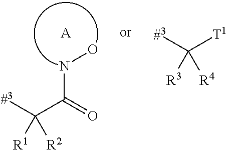
where
#³ marks the linkage site with the nitrogen atom,
R¹ is hydrogen,
R² is benzyl or 1H-indol-3-ylmethyl,
or
R¹ and R² together with the carbon atom to which they are bonded form a (1S,2R)-2-phenylcyclopropane-1,1-diyl group of the formula 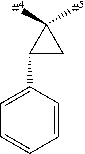
in which
#⁴ marks the linkage site with the adjacent nitrogen atom,
#⁵ marks the linkage site with the carbonyl group,
the ring A with the N—O moiety present therein is a mono- or bicyclic, optionally substituted heterocycle of the formula 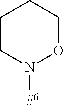
in which
#⁶ marks the linkage site with the carbonyl group,
R⁶ is hydrogen, hydroxy or benzyloxy,
R³ is hydrogen,
R⁴ is benzyl, 4-hydroxybenzyl or 1H-indol-3-ylmethyl,
or
R³ and R⁴ together with the carbon atom to which they are bonded form a (1S,2R)-2-phenylcyclopropane-1,1-diyl group of the formula 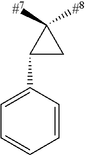
in which
#⁷ marks the linkage site with the adjacent nitrogen atom,
#⁸ marks the linkage site with the group T¹,
T¹ is a group of the formula —C(═O)—OR⁷ or —C(═O)—NR⁸R⁹,
in which
R⁷ is hydrogen,
R⁸ is hydrogen,
R⁹ is hydrogen,
R³⁵ is methyl,
and also their salts, solvates and solvates of the salts.

18. Process for preparing the compounds of the invention according to claim 1, of the general formula (Ia), characterized in that a solution of the binder in a buffer
[A] is admixed with a suitable reducing agent selected from the group consisting of dithiothreitol or tris(2-carboxyethyl)phosphine hydrochloride, and is subsequently reacted with a compound of the formula (II) 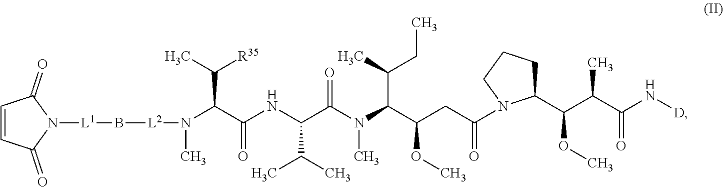
in which D, L¹, B, L² and R³⁵ each have the definitions indicated in claim 1,
to give a compound of the formula (I-A) 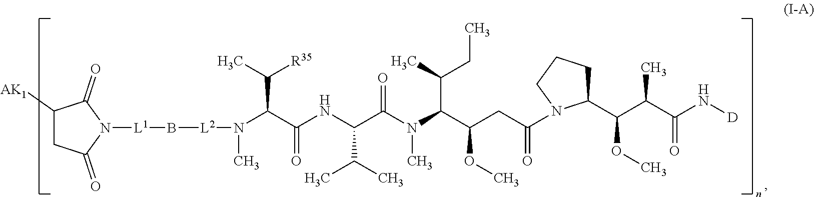
in which n, AK₁, D, L¹, B, L² and R³⁵ each have the definitions indicated in claim 1,
or
[B] is reacted with a compound of the formula (III) 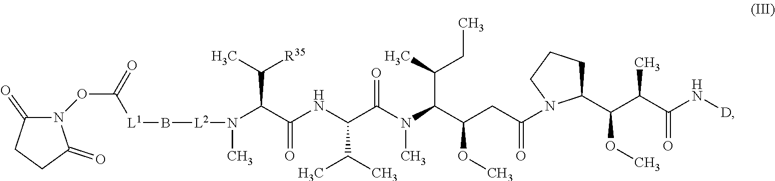
in which D, L¹, B, L² and R³⁵ each have the definitions indicated in claim 1,
to give a compound of the formula (I-B) 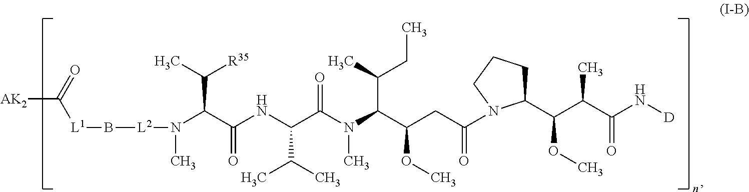
in which n, AK₂, D, L¹, B, L² and R³⁵ each have the definitions indicated in claim 1.

19. Compounds prepared by the process in claim 18, where AK₁ and AK₂ is an antibody which comprises the six CDR sequences of the antibody M048-D01-hIgG1 or M048-D01-hIgG1-b, the variable light and variable heavy chain of the antibody M048-D01-hIgG1 or M048-D01-hIgG1-b or the light and heavy chain of the antibody M048-D01-hIgG1 or M048-D01-hIgG1-b, and also their salts, solvates and solvates of the salts.

20. Binder-drug conjugate according to claim 1, where the binder binds specifically to FGFR2.

21. (canceled)

22. (canceled)

23. Binder-drug conjugate according to claim 1, where the binder binds to the extracellular N-terminal epitope (¹RPSFSLVEDTTLEPE¹⁵) of FGFR2.

24. (canceled)

25. Binder-drug conjugate according to claim 1, where the binder, after binding to FGFR2 on the target cell, is internalized by the binding of the target cell.

26. Binder-drug conjugate according to claim 1, where the binder is a binding protein, an antibody, or an antigen-binding antibody fragment.

27-31. (canceled)

32. Binder-drug conjugate according to claim 1, where the binder competes in binding to the cancer target molecule FGFR2 with the GAL-FR21, GAL-FR22 or M048-D01-hIgG1 antibody.

33. Binder-drug conjugate according to claim 1, where the binder comprises
the amino acid sequence of the CDR sequences of the variable light and heavy chain of the antibody M048-D01-hIgG1 represented in SEQ ID NO:15 (H-CDR1), SEQ ID NO:16 (H-CDR2), SEQ ID NO:17 (H-CDR3), SEQ ID NO:18 (L-CDR1), SEQ ID NO:19 (L-CDR2) and SEQ ID NO:20 (L-CDR3),
the amino acid sequence of the variable light and heavy chains of the antibody M048-D01-hIgG1, represented in SEQ ID NO:12 (Vl) and SEQ ID NO:11 (Vh),
the amino acid sequence of the variable light and heavy chains of the antibody M048-D01-hIgG1-b, represented in SEQ ID NO:14 (Vl) and SEQ ID NO:13 (Vh),
the amino acid sequence of the light and heavy chain of the antibody M048-D01-hIgG1-b represented in SEQ ID NO: 9 (light chain) and SEQ ID NO:10 (heavy chain),
the amino acid sequence of the light and heavy chain of the antibody M048-D01-hIgG1 represented in SEQ ID NO: 7 (light chain) and SEQ ID NO:8 (heavy chain), or
the amino acid sequence of the variable light and heavy chains of the antibody GAL-FR21 or GAL-FR22.

34-37. (canceled)

38. Medicament comprising a binder-drug conjugate or a compound according to claim 1, in combination with an inert, non-toxic, pharmaceutically suitable excipient.

39. Medicament comprising a binder-drug conjugate or a compound according to claim 1, in combination with one or more antihyperproliferative, cytostatic or cytotoxic substances.

40. (canceled)

41. Method for the treatment and/or prophylaxis of hyperproliferative and/or angiogenic diseases in humans and animals, using an effective amount of at least one binder-drug conjugate or a compound according to claim 1.


SEQUENCE LISTING (23 sequences)

SEQ ID NO: 1 - protein, 748 aa, Homo Sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 2 - protein, 659 aa, Homo Sapiens
RPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 3 - protein, 214 aa, Mus Musculus
NIVMTQSPKSMSMSVGDRVSITCKASQGVSNDVAWYQKKPGQSPKLLIYSASYRYTGVPDRFTGSGSGTDFTFTISNVQAEDLAVYYCQQHSTTPYTFGGGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 438 aa, Mus Musculus
QVQLQQPGAEVVKPGASVKMSCKASGYIFTTYNVHWVKQTPGQGLEWIGSIYPDNGDTSYNQNFKGKATLTADKSSSTAYIQLSSLTSEDSAVYYCARGDFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 5 - protein, 213 aa, Mus Musculus
DIQMTQSPSSLSASLGGRVTITCKASQDIKNYIAWYQHKPGKSPRLLIHYTSTLQPGVPSRFSGSGSGRDYSFSISNLEPEDIATYYCLQYDDLYMFGGGTKLDIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 450 aa, Mus Musculus
QVQLKQSGPGLVQPSQSLSITCTVSGFSLTSFGVHWVRQSPGKGLEWLGVIWSGGSTDYNADFRSRLSISKDNSKSQIFFKMNSLQPDDTAIYYCANFYYGYDDYVMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - protein, 216 aa, Homo Sapiens
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 452 aa, Homo Sapiens
QVELLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 216 aa, Homo Sapiens; identical to SEQ ID NO: 7

SEQ ID NO: 10 - protein, 451 aa, Homo Sapiens
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 122 aa, Homo Sapiens
QVELLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 12 - protein, 110 aa, Homo Sapiens
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 13 - protein, 122 aa, Homo Sapiens
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 14 - protein, 110 aa, Homo Sapiens; identical to SEQ ID NO: 12

SEQ ID NO: 15 - protein, 8 aa, Homo Sapiens
FSSYAMSW

SEQ ID NO: 16 - protein, 20 aa, Homo Sapiens
VSAISGSGTSTYYADSVKGR

SEQ ID NO: 17 - protein, 15 aa, Homo Sapiens
ARVRYNWNHGDWFDP

SEQ ID NO: 18 - protein, 13 aa, Homo Sapiens
SGSSSNIGNNYVS

SEQ ID NO: 19 - protein, 7 aa, Homo Sapiens
ENYNRPA

SEQ ID NO: 20 - protein, 12 aa, Homo Sapiens
CSSWDDSLNYWV

SEQ ID NO: 21 - protein, 122 aa, Homo Sapiens; identical to SEQ ID NO: 13

SEQ ID NO: 22 - protein, 110 aa, Homo Sapiens; identical to SEQ ID NO: 12

SEQ ID NO: 23 - protein, 15 aa, Homo Sapiens
RPSFSLVEDTTLEPE